(19) United States
(12) Patent Application Publication
(10) Pub. No.: US 2026/0097119 A1
(43) Pub. Date: Apr. 9, 2026

(54) COMPOSITIONS, SYSTEMS, AND METHODS FOR MODULATING T CELL FUNCTION

(71) Applicant: Tune Therapeutics, Inc., Seattle, WA (US)

(72) Inventors: Akiko SEKI, Durham, NC (US); Lucas James THOMPSON, Seattle, WA (US); Tyler S. KLANN, Durham, NC (US); Alexandra CROFT, Seattle, WA (US); Nathaniel CHARTRAND, Seattle, WA (US); Nicole DENAMUR, Seattle, WA (US); Andrew HILL, Seattle, WA (US); Jason DEAN, Seattle, WA (US); Emi TOKUDA, Seattle, WA (US); Blythe SATHER, Seattle, WA (US)

(73) Assignee: Tune Therapeutics, Inc., Seattle, WA (US)

(21) Appl. No.: 19/112,945

(22) PCT Filed: Sep. 18, 2023
(86) PCT No.: PCT/US2023/074515

§ 371 Date: Mar. 18, 2025

Related U.S. Application Data
(60) Provisional application No. 63/408,081, filed on Sep. 19, 2022.
(60) Provisional application No. 63/459,969, filed on Apr. 17, 2023.
(60) Provisional application No. 63/466,678, filed on May 15, 2023.
(60) Provisional application No. 63/530,030, filed on Jul. 31, 2023.
(60) Provisional application No. 63/532,345, filed on Aug. 11, 2023.
(60) Provisional application No. 63/581,949, filed on Sep. 11, 2023.

(51) Int. Cl. A61K 40/11; A61K 35/17; A61K 40/31; A61K 40/32; A61K 40/42; A61P 35/00; C12N 5/0783; C12N 5/10; C12N 9/22; C12N 15/113; C12N 15/62; C12N 15/85

(52) U.S. Cl. CPC A61K 40/11; A61K 35/17; A61K 40/31; A61K 40/32; A61K 40/42; A61P 35/00; C12N 5/0636; C12N 5/10; C12N 9/222; C12N 15/1136; C12N 15/62; C12N 15/85; A61K 2239/48; C12N 2310/20; C12N 2510/00; C12N 2750/14143

(57) ABSTRACT

Provided are epigenetic-modifying DNA-targeting systems, such as CRISPR-Cas/guide RNA (gRNA) systems, that bind to or target a target site in a gene or regulatory element thereof in a T cell. In some aspects, the provided epigenetic modifying DNA-targeting systems modulate a T cell function, such as a T cell phenotype or activity. In some aspects, also provided herein are methods and uses related to the provided compositions, for example in modulating T cells including in connection with methods of adoptive T cell therapy.

### CROSS-REFERENCE TO RELATED APPLICATIONS

This application claims priority from U.S. provisional application No. 63/408,081 filed Sep. 19, 2023, entitled “COMPOSITIONS, SYSTEMS, AND METHODS FOR MODULATING T CELL FUNCTION”, U.S. provisional application No. 63/459,969 filed Apr. 17, 2023, entitled “COMPOSITIONS, SYSTEMS, AND METHODS FOR MODULATING T CELL FUNCTION”, U.S. provisional application No. 63/466,678 filed May 15, 2023, entitled “COMPOSITIONS, SYSTEMS, AND METHODS FOR MODULATING T CELL FUNCTION”, U.S. provisional application No. 63/530,030 filed Jul. 31, 2023, entitled “COMPOSITIONS, SYSTEMS, AND METHODS FOR MODULATING T CELL FUNCTION”, U.S. provisional application No. 63/532,345 filed Aug. 11, 2023, entitled “COMPOSITIONS, SYSTEMS, AND METHODS FOR MODULATING T CELL FUNCTION”, and U.S. provisional application No. 63/581,949 filed Sep. 11, 2023, entitled “COMPOSITIONS, SYSTEMS, AND METHODS FOR MODULATING T CELL FUNCTION”, the contents of which are incorporated by reference in their entireties.

### INCORPORATION BY REFERENCE OF SEQUENCE LISTING

The present application is being filed along with a Sequence Listing in electronic format. The Sequence Listing is provided as a file entitled 224742002240.xml, created Sep. 18, 2023, which is 734,197 bytes in size. The information in the electronic format of the Sequence Listing is herein incorporated by reference in its entirety.

### FIELD

The present disclosure relates in some aspects to epigenetic-modifying DNA-targeting systems, such as CRISPR-Cas/guide RNA (gRNA) systems, that bind to or target a target site in a gene or regulatory element thereof in a T cell. In some aspects, the provided epigenetic modifying DNA-targeting systems of the present disclosure modulate a T cell function, such as a T cell phenotype or activity. In some aspects, the present disclosure is directed to methods and uses related to the provided compositions, for example in modulating T cells including in connection with methods of adoptive T cell therapy.

### BACKGROUND

The administration of T cells targeting a specific antigen, also known as Adoptive Cell Therapy (ACT), is a promising approach for treating diseases such as cancer. However, current ACT treatments face challenges including suboptimal T cell function, expansion, and persistence. Therefore, there is a need for new and improved methods to overcome these challenges. The present disclosure addresses these and other needs.

### SUMMARY

Provided herein is an epigenetic-modifying DNA-targeting system comprising at least one DNA-targeting module for repressing transcription of one or more genes in a T cell, wherein each of the at least one DNA-targeting module comprises a fusion protein comprising: (a) a DNA-binding domain capable of being targeted to a target site for one of the one or more genes, wherein the one or more genes are selected from the group consisting of CBLB, CCNC, CD5, CISH, DGKZ, ELOB, FAS, Fli1, GATA3, KDM1A, MED12, MYB, PRDM1, TGFBR2, and RASA2; and (b) at least one transcriptional repressor effector domain for repressing transcription of the one or more genes in a T cell.

In some aspects, provided herein is an epigenetic-modifying DNA-targeting system comprising at least one DNA-targeting module for repressing transcription of one or more genes in a T cell, wherein each of the at least one DNA-targeting module comprises a fusion protein comprising (a) a DNA-binding domain capable of being targeted to a target site in one of the one or more genes or regulatory DNA element thereof in a T cell, wherein the one or more genes are selected from the group consisting of CD5, KDM1A, CBLB, DGKZ, MYB, RASA2, ELOB, GATA3, CISH, PRDM1, MED12 and CCNC; and (b) at least one transcriptional repressor effector domain for repressing transcription of the one or more genes in a T cell.

Also provided herein is an epigenetic-modifying DNA-targeting system comprising at least one DNA-targeting module for increasing transcription of one or more genes in a T cell, wherein each of the at least one DNA-targeting module comprises a fusion protein comprising: (a) a DNA-binding domain capable of being targeted to a target site for one of the one or more genes, wherein the one or more genes are selected from the group consisting of BATF, CD28, EOMES, IL-2, IL2RB, IRF4, LAT, LCP2, TBX21, and VAV1; and (b) at least one transcriptional activator effector domain for increasing transcription of the one or more genes in a T cell.

In some aspects, provided herein is an epigenetic-modifying DNA-targeting system comprising at least one DNA-targeting module for increasing transcription of one or more genes in a T cell, wherein each of the at least one DNA-targeting module comprises a fusion protein comprising (a) a DNA-binding domain capable of being targeted to a target site in one of the one or more genes or regulatory DNA element thereof in a T cell, wherein the one or more genes are selected from the group consisting of VAV1, IL2 and IL2RB; and (b) at least one transcriptional activator effector domain for increasing transcription of the one or more genes in a T cell.

In some of any of the provided embodiments, transient delivery of the epigenetic-modifying DNA-targeting system to the T cell promotes increased T cell effector function upon T cell stimulation relative to the T cell effector function in the absence of the T cell stimulation. In some of any of the provided embodiments, the T cell effector function is characterized by an activity selected from the group consisting of IL-2 production, IFN-gamma production, TNF-alpha production, T cell proliferation or a combination of any of the foregoing. In some of any of the provided embodiments, the T cell effector function is characterized by IL-2 production. In some of any of the provided embodiments, the T cell effector function is characterized by IFN-gamma production. In some of any of the provided embodiments, the T cell effector function is characterized by IL-2 production and IFN-gamma production. In some of any of the provided embodiments, the T cell effector function is characterized by polyfunctional production of IL-2, IFN-gamma and TNF-alpha. In some of any of the provided embodiments, the T cell effector function is characterized by activity that further comprises T cell proliferation. In any of the embodiments herein, the T cell effector function is characterized by activity that further comprises killing of target cells. In any of the embodiments herein, the T cell effector function is characterized by activity that further comprises T cell persistence.

In some of any of the provided embodiments of an epigenetic-modifying DNA-targeting system, the increased T cell effector function is observed 48 hours or more after the transient delivery of the epigenetic-modifying DNA-targeting system to the T cell. In some of any of the provided embodiments, the increased T cell effector function is observed up to 6 days, up to 9 days, up to 12 days, up to 15 days, up to 21 days, up to 28 days, up to 35 days, up to 42 days, up to 49 days, up to 56 days, up to 63 days, up to 71 days or more after the transient delivery of the epigenetic-modifying DNA-targeting system to the T cell.

In some of any of the provided embodiments of an epigenetic-modifying DNA-targeting system, the T cell stimulation is with an anti-CD3 and anti-CD28 activation reagent.

In any of the embodiments herein, the T cell expresses an engineered antigen receptor, optionally a chimeric antigen receptor (CAR) or a T cell receptor (eTCR). In some embodiments, the engineered antigen receptor is a chimeric antigen receptor (CAR) or engineered T cell receptor (eTCR) directed against an antigen and the T cell stimulation is an antigen-specific stimulation of the CAR or eTCR, optionally wherein the T cell stimulation is with antigen-expressing target cells. In some of any of the provided embodiments of an epigenetic-modifying DNA-targeting system, the T cell expresses a chimeric antigen receptor (CAR) directed against an antigen and the T cell stimulation is an antigen-specific stimulation of the CAR, optionally wherein the T cell stimulation is with antigen-expressing target cells.

In some of any of the provided embodiments of an epigenetic-modifying DNA-targeting system, the T cell stimulation is a restimulation after at least one prior T cell stimulation of the T cells.

In some of any of the provided embodiments of an epigenetic-modifying DNA-targeting system, the DNA-targeting system does not introduce a genetic disruption or a DNA break.

In some of any of the provided embodiments of an epigenetic-modifying DNA-targeting system, the fusion protein of each DNA-targeting module comprises a DNA-binding domain selected from: a Clustered Regularly Interspaced Short Palindromic Repeats associated (Cas) protein or a variant thereof; a zinc finger protein (ZFP); a transcription activator-like effector (TALE); a meganuclease; a homing endonuclease; or an I-SceI enzyme or a variant thereof. In some of any such embodiments, the DNA-binding domain comprises a catalytically inactive variant of any of the foregoing.

In any of the embodiments herein, the at least one DNA-targeting module is a single DNA-targeting module that targets a target site for one of the one or more genes.

In some of any of the provided embodiments of an epigenetic-modifying DNA-targeting system, the at least one DNA-targeting module is a plurality of DNA-targeting modules for targeting a plurality of target sites of one or a plurality of the one or more genes or regulatory elements thereof. In any of the embodiments herein, the at least one DNA targeting module is a plurality of DNA-targeting modules for repressing transcription of one or more genes in a T cell, wherein each DNA-targeting module targets a target site for one of the one or more genes. In any of the embodiments herein, the at least one DNA targeting module is a plurality of DNA-targeting modules for increasing transcription of one or more genes in a T cell, wherein each DNA-targeting module targets a target site for one of the one or more genes. In some of any of the provided embodiments, the plurality of DNA-targeting modules is 2, 3, 4, 5 or 6 DNA-targeting modules.

In any of the embodiments herein, the plurality of DNA-targeting modules for repressing transcription of one or more genes in a T cell target at least a first gene and a second gene, wherein the first and second gene are independently selected from the group consisting of CBLB, CCNC, CD5, CISH, DGKZ, ELOB, FAS, Fli1, GATA3, KDM1A, MED12, MYB, PRDM1, TGFBR2, and RASA2. In some of any of the provided embodiments, the plurality of DNA-targeting modules target at least a first gene and a second gene, wherein the first and second gene are independently selected from the group consisting of CD5, KDM1A, CBLB, DGKZ, MYB, RASA2, ELOB, GATA3, CISH, PRDM1, MED12 and CCNC. In any of the embodiments herein, the plurality of DNA-targeting modules for repressing transcription of one or more genes in a T cell target at least a first gene and a second gene, wherein the first and second gene are independently selected from the group consisting of CBLB, CISH, MED12, MYB, PRDM1, and RASA2. In some embodiments, the plurality of DNA-targeting modules for repressing transcription of one or more genes in a T cell target at least a first gene, a second gene, and a third gene, wherein the first, second and third gene are independently selected from the group consisting of CBLB, CCNC, CD5, CISH, DGKZ, ELOB, FAS, Fli1, GATA3, KDM1A, MED12, MYB, PRDM1, TGFBR2, and RASA2. In some of any of the provided embodiments, the plurality of DNA-targeting modules target at least a first gene, a second gene, and a third gene, wherein the first, second and third gene are independently selected from the group consisting of CD5, KDM1A, CBLB, DGKZ, MYB, RASA2, ELOB, GATA3, CISH, PRDM1, MED12 and CCNC. In some embodiments, the plurality of DNA-targeting modules for repressing transcription of one or more genes in a T cell target at least a first gene, a second gene, and a third gene, wherein the first, second and third gene are independently selected from the group consisting of CBLB, CISH, MED12, MYB, PRDM1, and RASA2.

In any of the embodiments herein, the plurality of DNA-targeting modules target a combination of genes selected from: CBLB and CCNC; CBLB and CD5; CBLB and CISH; CBLB and DGKZ; CBLB and ELOB; CBLB and FAS; CBLB and Fli1; CBLB and GATA3; CBLB and KDM1A; CBLB and MED12; CBLB and MYB; CBLB and PRDM1; CBLB and RASA2; CD5 and CISH; CD5 and MYB; CISH and DGKZ; CISH and MYB; CISH and RASA2; GATA3 and CD5; GATA3 and CISH; GATA3 and MYB; MED12 and CBLB; MED12 and CD5; MED12 and CISH; MED12 and DGKZ; MED12 and ELOB; MED12 and GATA3; MED12 and MYB; MED12 and PRDM1; MED12 and RASA2; MYB and RASA2; PRDM1 and CISH; PRDM1 and GATA3; PRDM1 and MYB; PRDM1 and RASA2; CD5, CISH, and MYB; GATA3, CBLB, and MYB; GATA3, CD5, and MYB; PRDM1, GATA3, and CISH, TGFBR2 and MED12; and TGFBR2, MED12, and CISH. In some of any of the provided embodiments, the plurality of DNA-targeting modules target CBLB and MYB; CD5 and CISH; CD5, CISH and MYB; CD5 and MYB; CISH and DGKZ; CISH and MYB; CISH and RASA2; GATA3, CBLB and MYB; GATA3 and CD5; GATA3, CD5 and MYB; GATA3 and CISH; GATA3 and MYB; MYB and RASA2; PRDM1 and CISH; PRDM1 and GATA3; PRDM1, GATA3 and CISH; PRDM1 and MYB; PRDM1 and RASA2; CBLB and CCNC; and CBLB and MED12.

In any of the embodiments herein, the plurality of DNA-targeting modules target a combination of genes selected from: MED12 and CBLB; MED12 and CISH; CBLB and MYB; and CBLB and RASA2. In some embodiments, the first and second gene are CBLB and MYB. In some embodiments, the first and second gene are CBLB and MED12. In some embodiments, the first and second gene are CBLB and CCNC.

In any of the embodiments herein, the plurality of DNA-targeting modules for increasing transcription of one or more genes in a T cell target at least a first gene and a second gene, wherein the first and second gene are independently selected from the group consisting of BATF, CD28, EOMES, IL-2, IL2RB, IRF4, LAT, LCP2, TBX21, and VAV1. In some of any of the provided embodiments, the plurality of DNA-targeting modules target at least a first gene and a second gene, wherein the first and second gene are independently selected from the group consisting of VAV1, IL-2 and IL2RB. In any of the embodiments herein, the plurality of DNA-targeting modules for increasing transcription of one or more genes in a T cell target at least a first gene and a second gene, wherein the first and second gene are independently selected from the group consisting of EOMES, IL-2, LCP2, and TBX21. In any of the embodiments herein, the plurality of DNA-targeting modules for increasing transcription of one or more genes in a T cell target at least a first gene, a second gene, and a third gene, wherein the first, second and third gene are independently selected from the group consisting of BATF, CD28, EOMES, IL-2, IL2RB, IRF4, LAT, LCP2, TBX21, and VAV1. In any of the embodiments herein, the plurality of DNA-targeting modules for increasing transcription of one or more genes in a T cell target at least a first gene, a second gene, and a third gene, wherein the first, second and third gene are independently selected from the group consisting of EOMES, IL-2, LCP2, and TBX21.

In any of the embodiments herein, the plurality of DNA-targeting modules target a combination of genes selected from: BATF and IL-2; BATF and VAV1; CD28 and BATF; CD28 and EOMES; CD28 and IL-2; CD28 and LCP2; CD28 and TBX21; CD28 and VAV1; EOMES and BATF; EOMES and LCP2; EOMES and TBX21; EOMES and VAV1; EOMES and IL-2; LCP2 and BATF; LCP2 and IL-2; LCP2 and TBX21; LCP2 and VAV1; TBX21 and BATF; TBX21 and IL-2; TBX21 and TBX21; TBX21 and VAV1; and VAV1 and IL-2. In some of any of the provided embodiments, the first and second gene is IL2RB and VAV1. In some of any of the provided embodiments, the first and second gene is IL2 and VAV1. In some embodiments, the first and second gene are IL2 and LCP2. In some embodiments, the first and second gene are IL2 and TBX21. In some embodiments, the first and second gene are IL2 and EOMES.

In any of the embodiments herein, the target site for the gene or for each of the one or more genes is in the gene and/or a regulatory DNA element thereof. In some of any of the provided embodiments of an epigenetic-modifying DNA-targeting system, the target site of the gene or each of the one or more genes is in a regulatory DNA element thereof. In some of any of the provided embodiments, the regulatory DNA element is an enhancer or a promoter.

In any of the embodiments herein, the target site is within 1000 base pairs (bp) of a transcriptional start site of the gene. In some of any of the provided embodiments of an epigenetic-modifying DNA-targeting system, the target site is within 500 base pairs (bp) of a transcriptional start site of the gene.

In some of any of the provided embodiments of an epigenetic-modifying DNA-targeting system, the target site is selected from (a) a target site for CD5 having the sequence set forth in any one of SEQ ID NOS:1-3, a contiguous portion thereof of at least 14 nucleotides (nt), or a complementary sequence of any of the foregoing; (b) a target site for KDM1A having the sequence set forth in any one of SEQ ID NOS:4-6, a contiguous portion thereof of at least 14 nucleotides (nt), or a complementary sequence of any of the foregoing; (c) a target site for CBLB having the sequence set forth in any one of SEQ ID NOS:10-12, a contiguous portion thereof of at least 14 nucleotides (nt), or a complementary sequence of any of the foregoing; (d) a target site for DGKZ having the sequence set forth in any one of SEQ ID NOS:13-15, a contiguous portion thereof of at least 14 nucleotides (nt), or a complementary sequence of any of the foregoing; (e) a target site for MYB1 having the sequence set forth in any one of SEQ ID NOS:16-18, a contiguous portion thereof of at least 14 nucleotides (nt), or a complementary sequence of any of the foregoing; (f) a target site for RASA2 having the sequence set forth in any one of SEQ ID NOS:19-21, a contiguous portion thereof of at least 14 nucleotides (nt), or a complementary sequence of any of the foregoing; (g) a target site for ELOB having the sequence set forth in any one of SEQ ID NOS:22-24, a contiguous portion thereof of at least 14 nucleotides (nt), or a complementary sequence of any of the foregoing; (h) a target site for GATA3 having the sequence set forth in any one of SEQ ID NOS:25-27, a contiguous portion thereof of at least 14 nucleotides (nt), or a complementary sequence of any of the foregoing; (i) a target site for CISH having the sequence set forth in any one of SEQ ID NOS:28-30, a contiguous portion thereof of at least 14 nucleotides (nt), or a complementary sequence of any of the foregoing; j) a target site for PRDM1 having the sequence set forth in any one of SEQ ID NOS:31-33, a contiguous portion thereof of at least 14 nucleotides (nt), or a complementary sequence of any of the foregoing; (k) a target site for MED12 having the sequence set forth in any one of SEQ ID NOS:80-90, a contiguous portion thereof of at least 14 nucleotides (nt), or a complementary sequence of any of the foregoing; and (1) a target site for CCNC having the sequence set forth in any one of SEQ ID NOS:102-112, a contiguous portion thereof of at least 14 nucleotides (nt), or a complementary sequence of any of the foregoing; (m) a target site for FAS having the sequence set forth in any one of SEQ ID NOS:200-205 and 292-295, a contiguous portion thereof of at least 14 nucleotides (nt), or a complementary sequence of any of the foregoing; (n) a target site for Fli1 having the sequence set forth in any one of SEQ ID NOS:206-211, a contiguous portion thereof of at least 14 nucleotides (nt), or a complementary sequence of any of the foregoing; and (o) a target site for TGFBR2 having the sequence set forth in any one of SEQ ID NOS:300-302 and 306-308, a contiguous portion thereof of at least 14 nucleotides (nt), or a complementary sequence of any of the foregoing.

In some of any of the provided embodiments of an epigenetic-modifying DNA-targeting system, the target site is selected from (a) a target site for CD5 having the sequence set forth in any one of SEQ ID NOS:1-3 or a complementary sequence thereof; (b) a target site for KDM1A having the sequence set forth in any one of SEQ ID NOS:4-6 or a complementary sequence thereof; (c) a target site for CBLB having the sequence set forth in any one of SEQ ID NOS:10-12 or a complementary sequence thereof; (d) a target site for DGKZ having the sequence set forth in any one of SEQ ID NOS:13-15 or a complementary sequence of any of the foregoing; (e) a target site for MYB1 having the sequence set forth in any one of SEQ ID NOS:16-18 or a complementary sequence of any of the foregoing; (f) a target site for RASA2 having the sequence set forth in any one of SEQ ID NOS:19-21 or a complementary sequence of any of the foregoing; (g) a target site for ELOB having the sequence set forth in any one of SEQ ID NOS:22-24 or a complementary sequence of any of the foregoing; (h) a target site for GATA3 having the sequence set forth in any one of SEQ ID NOS:25-27 or a complementary sequence of any of the foregoing; (i) a target site for CISH having the sequence set forth in any one of SEQ ID NOS:28-30 or a complementary sequence of any of the foregoing; (j) a target site for PRDM1 having the sequence set forth in any one of SEQ ID NOS:31-33 or a complementary sequence of any of the foregoing; (k) a target site for MED12 having the sequence set forth in any one of SEQ ID NOS:80-90 or a complementary sequence of any of the foregoing; and (1) a target site for CCNC having the sequence set forth in any one of SEQ ID NOS:102-112 or a complementary sequence of any of the foregoing; (m) a target site for FAS having the sequence set forth in any one of SEQ ID NOS:200-205 and 292-295 or a complementary sequence of any of the foregoing; (n) a target site for Fli1 having the sequence set forth in any one of SEQ ID NOS:206-211 or a complementary sequence of any of the foregoing; and (o) a target site for TGFBR2 having the sequence set forth in any one of SEQ ID NOS:300-302 and 306-308 or a complementary sequence of any of the foregoing.

In any of the embodiments herein, (a) the target site is selected from a target site for CBLB having the sequence set forth in SEQ ID NO:11 or a complementary sequence thereof; (b) the target site is selected from a target site for MYB having the sequence set forth in SEQ ID NO:18 or a complementary sequence thereof; (c) the target site is selected from a target site for RASA2 having the sequence set forth in SEQ ID NO:19 or a complementary sequence thereof; (d) the target site is selected from a target site for CISH having the sequence set forth in SEQ ID NO:28 or a complementary sequence thereof; (e) the target site is selected from a target site for PRDM1 having the sequence set forth in SEQ ID NO:33 or a complementary sequence thereof; and (f) the target site is selected from a target site for MED12 having the sequence set forth in SEQ ID NO:81 or a complementary sequence thereof.

In any of the embodiments herein, the target site is selected from: (a) a target site for VAV1 having the sequence set forth in any one of SEQ ID NOS:7-9, 156, and 170, a contiguous portion thereof of at least 14 nucleotides (nt), or a complementary sequence of any of the foregoing; (b) a target site for IL2 having the sequence set forth in SEQ ID NO:78, a contiguous portion thereof of at least 14 nucleotides (nt), or a complementary sequence of any of the foregoing; (c) a target site for BATF having the sequence set forth in any one of SEQ ID NOS:172-174, a contiguous portion thereof of at least 14 nucleotides (nt), or a complementary sequence of any of the foregoing; (d) a target site for CD28 having the sequence set forth in any one of SEQ ID NOS:144-146 and 189-191, a contiguous portion thereof of at least 14 nucleotides (nt), or a complementary sequence of any of the foregoing; (e) a target site for EOMES having the sequence set forth in any one of SEQ ID NOS:147-149, a contiguous portion thereof of at least 14 nucleotides (nt), or a complementary sequence of any of the foregoing; (f) a target site for IRF4 having the sequence set forth in any one of SEQ ID NOS:175-177, a contiguous portion thereof of at least 14 nucleotides (nt), or a complementary sequence of any of the foregoing; (g) a target site for LAT having the sequence set forth in any one of SEQ ID NOS:184-186, a contiguous portion thereof of at least 14 nucleotides (nt), or a complementary sequence of any of the foregoing; (h) a target site for LCP2 having the sequence set forth in any one of SEQ ID NOS:150-152 and 187-188, a contiguous portion thereof of at least 14 nucleotides (nt), or a complementary sequence of any of the foregoing; and (i) a target site for TBX21 having the sequence set forth in any one of SEQ ID NOS:153-155, a contiguous portion thereof of at least 14 nucleotides (nt), or a complementary sequence of any of the foregoing.

In any of the embodiments herein, the target site is selected from: (a) a target site for VAV1 having the sequence set forth in any one of SEQ ID NOS:7-9, 156, and 170, or a complementary sequence of any of the foregoing; (b) a target site for IL2 having the sequence set forth in SEQ ID NO:78 or a complementary sequence of any of the foregoing; (c) a target site for BATF having the sequence set forth in any one of SEQ ID NOS:172-174, or a complementary sequence of any of the foregoing; (d) a target site for CD28 having the sequence set forth in any one of SEQ ID NOS:144-146 and 189-191, or a complementary sequence of any of the foregoing; (e) a target site for EOMES having the sequence set forth in any one of SEQ ID NOS:147-149, or a complementary sequence of any of the foregoing; (f) a target site for IRF4 having the sequence set forth in any one of SEQ ID NOS:175-177, or a complementary sequence of any of the foregoing; (g) a target site for LAT having the sequence set forth in any one of SEQ ID NOS:184-186, or a complementary sequence of any of the foregoing; (h) a target site for LCP2 having the sequence set forth in any one of SEQ ID NOS:150-152 and 187-188, or a complementary sequence of any of the foregoing; and (i) a target site for TBX21 having the sequence set forth in any one of SEQ ID NOS:153-155, or a complementary sequence of any of the foregoing.

In any of the embodiments herein, the target site is selected from: (a) a target site for IL-2 having the sequence set forth in SEQ ID NO:78, or a complementary sequence of any of the foregoing; (b) a target site for EOMES having the sequence set forth in SEQ ID NO:149, or a complementary sequence of any of the foregoing; (c) a target site for LCP2 having the sequence set forth in SEQ ID NO:151, or a complementary sequence of any of the foregoing; and (d) a target site for TBX21 having the sequence set forth in SEQ ID NO:155, or a complementary sequence of any of the foregoing.

In any of the embodiments herein, the DNA-binding domain of each of the at least one DNA-targeting module is a Clustered Regularly Interspaced Short Palindromic Repeats associated (Cas) protein or variant thereof, and each of the at least one DNA-targeting module further comprises at least one gRNA for targeting the DNA-binding domain to the target site of the one or more genes.

In any of the embodiments herein, the Cas protein or variant thereof is a deactivated (dCas) protein. In some embodiments, the dCas protein lacks nuclease activity. In some embodiments, the dCas protein is a dCas9 protein. In some embodiments, the dCas protein is a dCas12 protein. In some embodiments, the dCas9 protein is a Staphylococcus aureus dCas9 (dSaCas9) protein. In some embodiments, the dSaCas9 comprises at least one amino acid mutation selected from D10A and N580A, with reference to numbering of positions of SEQ ID NO:124. In some embodiments, the dSaCas9 protein comprises the sequence set forth in SEQ ID NO:125, or an amino acid sequence that has at least 90%, 91%, 92%, 93%, 94%, 95%, 96%, 97%, 98%, or 99% sequence identity thereto. In some embodiments, the dSaCas9 is set forth in SEQ ID NO:125. In some embodiments, the dCas9 protein is a Streptococcus pyogenes dCas9 (dSpCas9) protein. In some embodiments, the dSpCas9 protein comprises at least one amino acid mutation selected from D10A and H840A, with reference to numbering of positions of SEQ ID NO:126. In some embodiments, the dSpCas9 comprises the sequence set forth in SEQ ID NO:127, or an amino acid sequence that has at least 90%, 91%, 92%, 93%, 94%, 95%, 96%, 97%, 98%, or 99% sequence identity thereto. In some embodiments, the dSpCas9 is set forth in SEQ ID NO:127.

In any of the embodiments herein, the gRNA comprises a gRNA spacer that is complementary to the target site of the gene.

In any of the embodiments herein, the DNA-targeting module is for repressing transcription of the one or more genes and the gRNA is selected from: (a) a gRNA targeting a target site for CD5 and comprising a gRNA spacer sequence comprising the sequence set forth in any one of SEQ ID NOS:35-37, or a contiguous portion thereof of at least 14 nt; (b) a gRNA targeting a target site for KDM1A and comprising a gRNA spacer sequence comprising the sequence set forth in any one of SEQ ID NOS:38-40, or a contiguous portion thereof of at least 14 nt; (c) a gRNA targeting a target site for CBLB and comprising a gRNA spacer sequence comprising the sequence set forth in any one of SEQ ID NOS:44-46, or a contiguous portion thereof of at least 14 nt; (d) a gRNA targeting a target site for DGKZ and comprising a gRNA spacer sequence comprising the sequence set forth in any one of SEQ ID NOS:47-49, or a contiguous portion thereof of at least 14 nt; (e) a gRNA targeting a target site for MYB and comprising a gRNA spacer sequence comprising the sequence set forth in any one of SEQ ID NOS:50-52, or a contiguous portion thereof of at least 14 nt; (f) a gRNA targeting a target site for RASA2 and comprising a gRNA spacer sequence comprising the sequence set forth in any one of SEQ ID NOS:53-55, or a contiguous portion thereof of at least 14 nt; (g) a gRNA targeting a target site for ELOB and comprising a gRNA spacer sequence comprising the sequence set forth in any one of SEQ ID NOS:56-58, or a contiguous portion thereof of at least 14 nt; (h) a gRNA targeting a target site for GATA3 and comprising a gRNA spacer sequence comprising the sequence set forth in any one of SEQ ID NOS:59-61, or a contiguous portion thereof of at least 14 nt; (i) a gRNA targeting a target site for CISH and comprising a gRNA spacer sequence comprising the sequence set forth in any one of SEQ ID NOS:62-64, or a contiguous portion thereof of at least 14 nt; (j) a gRNA targeting a target site for PRDM1 and comprising a gRNA spacer sequence comprising the sequence set forth in any one of SEQ ID NOS:65-67, or a contiguous portion thereof of at least 14 nt; (k) a gRNA targeting a target site for MED12 and comprising a gRNA spacer sequence comprising the sequence set forth in any one of SEQ ID NOS:91-101, or a contiguous portion thereof of at least 14 nt; (1) a gRNA targeting a target site for CCNC and comprising a gRNA spacer sequence comprising the sequence set forth in any one of SEQ ID NOS:113-123, or a contiguous portion thereof of at least 14 nt; (m) a gRNA targeting a target site for FAS and comprising a gRNA spacer sequence comprising the sequence set forth in any one of SEQ ID NOS:212-217 and 296-299, or a contiguous portion thereof of at least 14 nt; (n) a gRNA targeting a target site for Fli1 and comprising a gRNA spacer sequence comprising the sequence set forth in any one of SEQ ID NOS:218-223, or a contiguous portion thereof of at least 14 nt; and (o) a gRNA targeting a target site for TGFBR2 and comprising a gRNA spacer sequence comprising the sequence set forth in any one of SEQ ID NOS:303-305 and 309-311, or a contiguous portion thereof of at least 14 nt.

In any of the embodiments herein, the DNA-targeting module is for repressing transcription of the one or more genes and the gRNA is selected from: (a) a gRNA targeting a target site for CD5 and comprising a gRNA spacer sequence set forth in any one of SEQ ID NOS:35-37; (b) a gRNA targeting a target site for KDM1A and comprising a gRNA spacer sequence set forth in any one of SEQ ID NOS:38-40; (c) a gRNA targeting a target site for CBLB and comprising a gRNA spacer sequence set forth in any one of SEQ ID NOS:44-46; (d) a gRNA targeting a target site for DGKZ and comprising a gRNA spacer sequence set forth in any one of SEQ ID NOS:47-49; (e) a gRNA targeting a target site for MYB and comprising a gRNA spacer sequence set forth in any one of SEQ ID NOS:50-52; (f) a gRNA targeting a target site for RASA2 and comprising a gRNA spacer sequence set forth in any one of SEQ ID NOS:53-55; (g) a gRNA targeting a target site for ELOB and comprising a gRNA spacer sequence set forth in any one of SEQ ID NOS:56-58; (h) a gRNA targeting a target site for GATA3 and comprising a gRNA spacer sequence set forth in any one of SEQ ID NOS:59-61; (i) a gRNA targeting a target site for CISH and comprising a gRNA spacer sequence set forth in any one of SEQ ID NOS:62-64; (j) a gRNA targeting a target site for PRDM1 and comprising a gRNA spacer sequence set forth in any one of SEQ ID NOS:65-67; (k) a gRNA targeting a target site for MED12 and comprising a gRNA spacer sequence set forth in any one of SEQ ID NOS:91-101; (1) a gRNA targeting a target site for CCNC and comprising a gRNA spacer sequence set forth in any one of SEQ ID NOS:113-123; (m) a gRNA targeting a target site for FAS and comprising a gRNA spacer sequence set forth in any one of SEQ ID NOS:212-217 and 296-299; (n) a gRNA targeting a target site for Fli1 and comprising a gRNA spacer sequence set forth in any one of SEQ ID NOS:218-223; and (o) a gRNA targeting a target site for TGFBR2 and comprising a gRNA spacer sequence set forth in any one of SEQ ID NOS:303-305 and 309-311.

In any of the embodiments herein, the DNA-targeting module is for repressing transcription of the one or more genes and the gRNA is selected from: (a) a gRNA targeting a target site for CBLB and comprising a gRNA spacer sequence set forth in SEQ ID NO:45; (b) a gRNA targeting a target site for MYB and comprising a gRNA spacer sequence set forth in SEQ ID NO:52; (c) a gRNA targeting a target site for RASA2 and comprising a gRNA spacer sequence set forth in SEQ ID NO:53; (d) a gRNA targeting a target site for CISH and comprising a gRNA spacer sequence set forth in SEQ ID NO:62; (e) a gRNA targeting a target site for PRDM1 and comprising a gRNA spacer sequence set forth in SEQ ID NO:67; and (f) a gRNA targeting a target site for MED12 and comprising a gRNA spacer sequence set forth in SEQ ID NO:92.

In any of the embodiments herein, the DNA-targeting module is for increasing transcription of the one or more genes and the gRNA is selected from: (a) a gRNA targeting a target site for VAV1 and comprising a gRNA spacer sequence comprising the sequence set forth in any one of SEQ ID NOS:41-43, 169, and 171, or a contiguous portion thereof of at least 14 nt; (b) a gRNA targeting a target site for IL2 and comprising a gRNA spacer sequence comprising the sequence set forth in SEQ ID NO:79, or a contiguous portion thereof of at least 14 nt; (c) a gRNA targeting a target site for BATF and comprising a gRNA spacer sequence comprising the sequence set forth in any one of SEQ ID NOS:178-180, or a contiguous portion thereof of at least 14 nt; (d) a gRNA targeting a target site for CD28 and comprising a gRNA spacer sequence comprising the sequence set forth in any one of SEQ ID NOS:157-159 and 197-199, or a contiguous portion thereof of at least 14 nt; (e) a gRNA targeting a target site for EOMES and comprising a gRNA spacer sequence comprising the sequence set forth in any one of SEQ ID NOS:160-162, or a contiguous portion thereof of at least 14 nt; (f) a gRNA targeting a target site for IRF4 and comprising a gRNA spacer sequence comprising the sequence set forth in any one of SEQ ID NOS:181-183, or a contiguous portion thereof of at least 14 nt; (g) a gRNA targeting a target site for LAT and comprising a gRNA spacer sequence comprising the sequence set forth in any one of SEQ ID NOS:192-194, or a contiguous portion thereof of at least 14 nt; (h) a gRNA targeting a target site for LCP2 and comprising a gRNA spacer sequence comprising the sequence set forth in any one of SEQ ID NOS:163-165 and 195-196, or a contiguous portion thereof of at least 14 nt; and (i) a gRNA targeting a target site for TBX21 and comprising a gRNA spacer sequence comprising the sequence set forth in any one of SEQ ID NOS:166-168, or a contiguous portion thereof of at least 14 nt.

In any of the embodiments herein, the DNA-targeting module is for increasing transcription of the one or more genes and the gRNA is selected from: (a) a gRNA targeting a target site for VAV1 and comprising a gRNA spacer sequence set forth in any one of SEQ ID NOS:41-43, 169, and 171; (b) a gRNA targeting a target site for IL2 and comprising a gRNA spacer sequence set forth in SEQ ID NO:79; (c) a gRNA targeting a target site for BATF and comprising a gRNA spacer sequence set forth in any one of SEQ ID NOS:178-180; (d) a gRNA targeting a target site for CD28 and comprising a gRNA spacer sequence set forth in any one of SEQ ID NOS:157-159 and 197-199; (e) a gRNA targeting a target site for EOMES and comprising a gRNA spacer sequence set forth in any one of SEQ ID NOS:160-162; (f) a gRNA targeting a target site for IRF4 and comprising a gRNA spacer sequence set forth in any one of SEQ ID NOS:181-183; (g) a gRNA targeting a target site for LAT and comprising a gRNA spacer sequence set forth in any one of SEQ ID NOS:192-194; (h) a gRNA targeting a target site for LCP2 and comprising a gRNA spacer sequence set forth in any one of SEQ ID NOS:163-165 and 195-196; and (i) a gRNA targeting a target site for TBX21 and comprising a gRNA spacer sequence set forth in any one of SEQ ID NOS:166-168.

In any of the embodiments herein, the DNA-targeting module is for increasing transcription of the one or more genes and the gRNA is selected from: (a) a gRNA targeting a target site for IL-2 and comprising a gRNA spacer sequence set forth in SEQ ID NO:79; (a) a gRNA targeting a target site for EOMES and comprising a gRNA spacer sequence set forth in SEQ ID NO:162; (a) a gRNA targeting a target site for LCP2 and comprising a gRNA spacer sequence set forth in SEQ ID NO:164; and (a) a gRNA targeting a target site for TBX21 and comprising a gRNA spacer sequence set forth in SEQ ID NO:168.

In any of the embodiments herein, the gRNA comprises a spacer sequence between 14 nt and 24 nt, or between 16 nt and 22 nt in length. In any of the embodiments herein, the gRNA comprises a spacer sequence that is 18 nt, 19 nt, 20 nt, 21 nt, or 22 nt in length.

In any of the embodiments herein, the gRNA further comprises a scaffold sequence set forth in SEQ ID NO:69.

In any of the embodiments herein, the at least one transcriptional repressor effector domain is capable of reducing transcription of the one or more genes.

In any of the embodiments herein, the transcriptional repressor effector domain is selected from the group consisting of a KRAB domain, a DNMT3A domain, a DNMT3L domain, a DNMT3B domain, a DNMT3A-DNMT3L fusion protein domain, an ERF repressor domain, an Mxil repressor domain, a SID4X repressor domain, a Mad-SID repressor domain, an LSD1 repressor domain, an EZH2 repressor domain, a SunTag domain, or a variant or portion of any of the foregoing, or a combination of any of the foregoing.

In any of the embodiments herein, the transcriptional repressor effector domain is a KRAB domain, a DNMT3A domain, or a DNMT3L domain, or a combination of any of the foregoing.

In any of the embodiments herein, the at least one transcriptional repressor effector domain comprises a KRAB domain or a variant or portion thereof that exhibits transcriptional repressor activity. In any of the embodiments herein, the at least one transcriptional repressor effector domain comprises the sequence set forth in any one of SEQ ID NOS:70, 235, and 355-358, a portion thereof, or an amino acid sequence that has at least 90%, 91%, 92%, 93%, 94%, 95%, 96%, 97%, 98%, or 99% sequence identity to any of the foregoing.

In any of the embodiments herein, the at least one transcriptional repressor effector domain comprises a DNMT3A domain or a variant or portion thereof that exhibits transcriptional repressor activity. In any of the embodiments herein, the at least one transcriptional repressor domain comprises the sequence set forth in SEQ ID NO:131 or 238, a portion thereof, or an amino acid sequence that has at least 90%, 91%, 92%, 93%, 94%, 95%, 96%, 97%, 98%, or 99% sequence identity to any of the foregoing.

In any of the embodiments herein, the at least one transcriptional repressor domain comprises a DNMT3L domain or a variant or portion thereof that exhibits transcriptional repressor activity. In any of the embodiments herein, the at least one transcriptional repressor domain comprises the sequence set forth in any one of SEQ ID NOS:133 and 240-242, a portion thereof, or an amino acid sequence that has at least 90%, 91%, 92%, 93%, 94%, 95%, 96%, 97%, 98%, or 99% sequence identity to any of the foregoing.

In any of the embodiments herein, the at least one transcriptional repressor domain is a DNMT3A-DNMT3L fusion protein domain, a DNMT3B-DNMT3L fusion protein domain, or a variant thereof that exhibits transcriptional repressor activity. In any of the embodiments herein, the at least one transcriptional repressor domain comprises the sequence set forth in any one of SEQ ID NOS:135, 137, or 363, a portion thereof, or an amino acid sequence that has at least 90%, 91%, 92%, 93%, 94%, 95%, 96%, 97%, 98%, or 99% sequence identity to any of the foregoing. In any of the embodiments herein, the fusion protein comprises the sequence set forth in any one of SEQ ID NOS:138-141, 332-351, and 365-384, a portion thereof, or an amino acid sequence that has at least 90%, 91%, 92%, 93%, 94%, 95%, 96%, 97%, 98%, or 99% sequence identity thereto.

In any of the embodiments herein, the at least one transcriptional activator effector domain is capable of increasing transcription of the one or more genes.

In any of the embodiments herein, the at least one transcriptional activator effector domain is selected from the group consisting of: a VP64 domain, a p65 activation domain, a p300 domain, an Rta domain, a CBP domain, a VPR domain, a VPH domain, an HSF1 domain, a TET protein domain, optionally wherein the TET protein is TET1, a SunTag domain, or a domain, portion, variant, or truncation of any of the foregoing.

In any of the embodiments herein, the at least one transcriptional activator effector domain comprises at least one VP16 domain, and/or a VP16 tetramer (“VP64”) or a variant thereof. In any of the embodiments herein, the at least one transcriptional activator effector domain comprises a VP64 domain or a variant or portion thereof that exhibits transcriptional activation activity. In any of the embodiments herein, the at least one transcriptional activator effector domain is VP64. In any of the embodiments herein, the at least one transcriptional activator effector domain comprises the sequence set forth in SEQ ID NO: 142, a portion thereof, or an amino acid sequence that has at least 90%, 91%, 92%, 93%, 94%, 95%, 96%, 97%, 98%, or 99% sequence identity to any of the foregoing. In any of the embodiments herein, the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

Also provided herein is a combination of epigenetic-modifying DNA-targeting systems comprising at least two of the DNA-targeting systems described herein, wherein each DNA-targeting system represses transcription of a different gene of the one or more genes.

Also provided herein is a combination of epigenetic-modifying DNA-targeting systems comprising at least two of the DNA-targeting systems described herein, wherein each DNA-targeting system increases transcription of a different gene of the one or more genes.

Also provided herein is a guide RNA (gRNA) that targets a target site for a gene selected from the group consisting of CBLB, CCNC, CD5, CISH, DGKZ, ELOB, FAS, Fli1, GATA3, KDM1A, MED12, MYB, PRDM1, TGFBR2, and RASA2. In some embodiments, the target site for the gene is in the gene or a regulatory DNA element thereof. In some embodiments, the regulatory DNA element is an enhancer or a promoter. In some embodiments, the target site is within 1000 base pairs (bp) of a transcriptional start site of the gene. In some embodiments, the target site is within 500 base pairs (bp) of a transcriptional start site of the gene. In any of the embodiments herein, the target site is selected from: (a) a target site for CD5 having the sequence set forth in any one of SEQ ID NOS:1-3, a contiguous portion thereof of at least 14 nucleotides (nt), or a complementary sequence of any of the foregoing; (b) a target site for KDM1A having the sequence set forth in any one of SEQ ID NOS:4-6, a contiguous portion thereof of at least 14 nucleotides (nt), or a complementary sequence of any of the foregoing; (c) a target site for CBLB having the sequence set forth in any one of SEQ ID NOS:10-12, a contiguous portion thereof of at least 14 nucleotides (nt), or a complementary sequence of any of the foregoing; (d) a target site for DGKZ having the sequence set forth in any one of SEQ ID NOS:13-15, a contiguous portion thereof of at least 14 nucleotides (nt), or a complementary sequence of any of the foregoing; (e) a target site for MYB having the sequence set forth in any one of SEQ ID NOS:16-18, a contiguous portion thereof of at least 14 nucleotides (nt), or a complementary sequence of any of the foregoing; (f) a target site for RASA2 having the sequence set forth in any one of SEQ ID NOS:19-21, a contiguous portion thereof of at least 14 nucleotides (nt), or a complementary sequence of any of the foregoing; (g) a target site for ELOB having the sequence set forth in any one of SEQ ID NOS:22-24, a contiguous portion thereof of at least 14 nucleotides (nt), or a complementary sequence of any of the foregoing; (h) a target site for GATA3 having the sequence set forth in any one of SEQ ID NOS:25-27, a contiguous portion thereof of at least 14 nucleotides (nt), or a complementary sequence of any of the foregoing; (i) a target site for CISH having the sequence set forth in any one of SEQ ID NOS:28-30, a contiguous portion thereof of at least 14 nucleotides (nt), or a complementary sequence of any of the foregoing; (j) a target site for PRDM1 having the sequence set forth in any one of SEQ ID NOS:31-33, a contiguous portion thereof of at least 14 nucleotides (nt), or a complementary sequence of any of the foregoing; (k) a target site for MED12 having the sequence set forth in any one of SEQ ID NOS:80-90, a contiguous portion thereof of at least 14 nucleotides (nt), or a complementary sequence of any of the foregoing; (1) a target site for CCNC having the sequence set forth in any one of SEQ ID NOS:102-112, a contiguous portion thereof of at least 14 nucleotides (nt), or a complementary sequence of any of the foregoing; (m) a target site for FAS having the sequence set forth in any one of SEQ ID NOS:200-205 and 292-295, a contiguous portion thereof of at least 14 nucleotides (nt), or a complementary sequence of any of the foregoing; (n) a target site for Fli1 having the sequence set forth in any one of SEQ ID NOS:206-211, a contiguous portion thereof of at least 14 nucleotides (nt), or a complementary sequence of any of the foregoing; and (o) a target site for TGFBR2 having the sequence set forth in any one of SEQ ID NOS:300-302 and 306-308, a contiguous portion thereof of at least 14 nucleotides (nt), or a complementary sequence of any of the foregoing.

In any of the embodiments herein, the target site is selected from: (a) a target site for CD5 having the sequence set forth in any one of SEQ ID NOS:1-3 or a complementary sequence thereof; (b) a target site for KDM1A having the sequence set forth in any one of SEQ ID NOS:4-6 or a complementary sequence thereof; (c) a target site for CBLB having the sequence set forth in any one of SEQ ID NOS:10-12 or a complementary sequence thereof; (d) a target site for DGKZ having the sequence set forth in any one of SEQ ID NOS:13-15 or a complementary sequence of any of the foregoing; (e) a target site for MYB having the sequence set forth in any one of SEQ ID NOS:16-18 or a complementary sequence of any of the foregoing; (f) a target site for RASA2 having the sequence set forth in any one of SEQ ID NOS:19-21 or a complementary sequence of any of the foregoing; (g) a target site for ELOB having the sequence set forth in any one of SEQ ID NOS:22-24 or a complementary sequence of any of the foregoing; (h) a target site for GATA3 having the sequence set forth in any one of SEQ ID NOS:25-27 or a complementary sequence of any of the foregoing; (i) a target site for CISH having the sequence set forth in any one of SEQ ID NOS:28-30 or a complementary sequence of any of the foregoing; (j) a target site for PRDM1 having the sequence set forth in any one of SEQ ID NOS:31-33 or a complementary sequence of any of the foregoing; (k) a target site for MED12 having the sequence set forth in any one of SEQ ID NOS:80-90 or a complementary sequence of any of the foregoing; (1) a target site for CCNC having the sequence set forth in any one of SEQ ID NOS:102-112 or a complementary sequence of any of the foregoing; (m) a target site for FAS having the sequence set forth in any one of SEQ ID NOS:200-205 and 292-295 or a complementary sequence of any of the foregoing; (n) a target site for Fli1 having the sequence set forth in any one of SEQ ID NOS:206-211 or a complementary sequence of any of the foregoing; and (o) a target site for TGFBR2 having the sequence set forth in any one of SEQ ID NOS:300-302 and 306-308 or a complementary sequence of any of the foregoing.

In any of the embodiments herein, the target site is selected from: (a) a target site for CBLB having the sequence set forth in SEQ ID NO:11 or a complementary sequence of any of the foregoing; (b) a target site for MYB having the sequence set forth in SEQ ID NO:18 or a complementary sequence of any of the foregoing. (c) a target site for RASA2 having the sequence set forth in SEQ ID NO:19 or a complementary sequence of any of the foregoing; (d) a target site for CISH having the sequence set forth in SEQ ID NO:28 or a complementary sequence of any of the foregoing; (e) a target site for PRDM1 having the sequence set forth in SEQ ID NO:33 or a complementary sequence of any of the foregoing; and (f) a target site for MED12 having the sequence set forth in SEQ ID NO:81 or a complementary sequence of any of the foregoing. In any of the embodiments herein, the gRNA is selected from: (a) a gRNA targeting a target site for CD5 and comprising a gRNA spacer sequence comprising the sequence set forth in any one of SEQ ID NOS:35-37, or a contiguous portion thereof of at least 14 nt; (b) a gRNA targeting a target site for KDM1A and comprising a gRNA spacer sequence comprising the sequence set forth in any one of SEQ ID NOS:38-40, or a contiguous portion thereof of at least 14 nt; (c) a gRNA targeting a target site for CBLB and comprising a gRNA spacer sequence comprising the sequence set forth in any one of SEQ ID NOS:44-46, or a contiguous portion thereof of at least 14 nt; (d) a gRNA targeting a target site for DGKZ and comprising a gRNA spacer sequence comprising the sequence set forth in any one of SEQ ID NOS:47-49, or a contiguous portion thereof of at least 14 nt; (e) a gRNA targeting a target site for MYB and comprising a gRNA spacer sequence comprising the sequence set forth in any one of SEQ ID NOS:50-52, or a contiguous portion thereof of at least 14 nt; (f) a gRNA targeting a target site for RASA2 and comprising a gRNA spacer sequence comprising the sequence set forth in any one of SEQ ID NOS:53-55, or a contiguous portion thereof of at least 14 nt; (g) a gRNA targeting a target site for ELOB and comprising a gRNA spacer sequence comprising the sequence set forth in any one of SEQ ID NOS:56-58, or a contiguous portion thereof of at least 14 nt; (h) a gRNA targeting a target site for GATA3 and comprising a gRNA spacer sequence comprising the sequence set forth in any one of SEQ ID NOS:59-61, or a contiguous portion thereof of at least 14 nt; (i) a gRNA targeting a target site for CISH and comprising a gRNA spacer sequence comprising the sequence set forth in any one of SEQ ID NOS:62-64, or a contiguous portion thereof of at least 14 nt; (j) a gRNA targeting a target site for PRDM1 and comprising a gRNA spacer sequence comprising the sequence set forth in any one of SEQ ID NOS:65-67, or a contiguous portion thereof of at least 14 nt; (k) a gRNA targeting a target site for MED12 and comprising a gRNA spacer sequence comprising the sequence set forth in any one of SEQ ID NOS:91-101, or a contiguous portion thereof of at least 14 nt; (1) a gRNA targeting a target site for CCNC and comprising a gRNA spacer sequence comprising the sequence set forth in any one of SEQ ID NOS:113-123, or a contiguous portion thereof of at least 14 nt; (m) a gRNA targeting a target site for FAS and comprising a gRNA spacer sequence comprising the sequence set forth in any one of SEQ ID NOS:212-217 and 296-299, or a contiguous portion thereof of at least 14 nt; (n) a gRNA targeting a target site for Fli1 and comprising a gRNA spacer sequence comprising the sequence set forth in any one of SEQ ID NOS:218-223, or a contiguous portion thereof of at least 14 nt; and (o) a gRNA targeting a target site for TGFBR2 and comprising a gRNA spacer sequence comprising the sequence set forth in any one of SEQ ID NOS:303-305 and 309-311, or a contiguous portion thereof of at least 14 nt.

In any of the embodiments herein, the gRNA is selected from: (a) a gRNA targeting a target site for CD5 and comprising a gRNA spacer sequence set forth in any one of SEQ ID NOS:35-37; (b) a gRNA targeting a target site for KDM1A and comprising a gRNA spacer sequence set forth in any one of SEQ ID NOS:38-40; (c) a gRNA targeting a target site for CBLB and comprising a gRNA spacer sequence set forth in any one of SEQ ID NOS:44-46; (d) a gRNA targeting a target site for DGKZ and comprising a gRNA spacer sequence set forth in any one of SEQ ID NOS:47-49; (e) a gRNA targeting a target site for MYB and comprising a gRNA spacer sequence set forth in any one of SEQ ID NOS:50-52; (f) a gRNA targeting a target site for RASA2 and comprising a gRNA spacer sequence set forth in any one of SEQ ID NOS:53-55; (g) a gRNA targeting a target site for ELOB and comprising a gRNA spacer sequence set forth in any one of SEQ ID NOS:56-58; (h) a gRNA targeting a target site for GATA3 and comprising a gRNA spacer sequence set forth in any one of SEQ ID NOS:59-61; (i) a gRNA targeting a target site for CISH and comprising a gRNA spacer sequence set forth in any one of SEQ ID NOS:62-64; (j) a gRNA targeting a target site for PRDM1 and comprising a gRNA spacer sequence set forth in any one of SEQ ID NOS:65-67; (k) a gRNA targeting a target site for MED12 and comprising a gRNA spacer sequence set forth in any one of SEQ ID NOS:91-101; (1) a gRNA targeting a target site for CCNC and comprising a gRNA spacer sequence set forth in any one of SEQ ID NOS:113-123; (m) a gRNA targeting a target site for FAS and comprising a gRNA spacer sequence set forth in any one of SEQ ID NOS:212-217 and 296-299; (n) a gRNA targeting a target site for Fli1 and comprising a gRNA spacer sequence set forth in any one of SEQ ID NOS:218-223; and (o) a gRNA targeting a target site for TGFBR2 and comprising a gRNA spacer sequence set forth in any one of SEQ ID NOS:303-305 and 309-311.

In any of the embodiments herein, the gRNA is selected from: (a) a gRNA targeting a target site for CBLB and comprising a gRNA spacer sequence set forth in SEQ ID NO:45; (b) a gRNA targeting a target site for MYB and comprising a gRNA spacer sequence set forth in SEQ ID NO:52; (c) a gRNA targeting a target site for RASA2 and comprising a gRNA spacer sequence set forth in SEQ ID NO:53; (d) a gRNA targeting a target site for CISH and comprising a gRNA spacer sequence set forth in SEQ ID NO:62; (e) a gRNA targeting a target site for PRDM1 and comprising a gRNA spacer sequence set forth in SEQ ID NO:67; and (f) a gRNA targeting a target site for MED12 and comprising a gRNA spacer sequence set forth in SEQ ID NO:91.

In any of the embodiments herein, the gRNA comprises a spacer sequence between 14 nt and 24 nt, or between 16 nt and 22 nt in length. In any of the embodiments herein, the gRNA comprises a spacer sequence that is 18 nt, 19 nt, 20 nt, 21 nt, or 22 nt in length.

In any of the embodiments herein, the gRNA further comprises a scaffold sequence set forth in SEQ ID NO:69.

Also provided herein is a guide RNA (gRNA) that targets a target site for a gene selected from the group consisting of BATF, CD28, EOMES, IL-2, IL2RB, IRF4, LAT, LCP2, TBX21, and VAV1. In some embodiments, the target site for the gene is in the gene or a regulatory DNA element thereof. In some embodiments, the regulatory DNA element is an enhancer or a promoter.

In any of the embodiments herein, the target site is within 1000 base pairs (bp) of a transcriptional start site of the gene. In any of the embodiments herein, the target site is within 500 base pairs (bp) of a transcriptional start site of the gene.

In any of the embodiments herein, the target site is selected from: (a) a target site for VAV1 having the sequence set forth in any one of SEQ ID NOS:7-9, 156, and 170, a contiguous portion thereof of at least 14 nucleotides (nt), or a complementary sequence of any of the foregoing; (b) a target site for IL2 having the sequence set forth in SEQ ID NO:78, a contiguous portion thereof of at least 14 nucleotides (nt), or a complementary sequence of any of the foregoing; (c) a target site for BATF having the sequence set forth in any one of SEQ ID NOS:172-174, a contiguous portion thereof of at least 14 nucleotides (nt), or a complementary sequence of any of the foregoing; (d) a target site for CD28 having the sequence set forth in any one of SEQ ID NOS:144-146 and 189-191, a contiguous portion thereof of at least 14 nucleotides (nt), or a complementary sequence of any of the foregoing; (e) a target site for EOMES having the sequence set forth in any one of SEQ ID NOS:147-149, a contiguous portion thereof of at least 14 nucleotides (nt), or a complementary sequence of any of the foregoing; (f) a target site for IRF4 having the sequence set forth in any one of SEQ ID NOS:175-177, a contiguous portion thereof of at least 14 nucleotides (nt), or a complementary sequence of any of the foregoing; (g) a target site for LAT having the sequence set forth in any one of SEQ ID NOS:184-186, a contiguous portion thereof of at least 14 nucleotides (nt), or a complementary sequence of any of the foregoing; (h) a target site for LCP2 having the sequence set forth in any one of SEQ ID NOS:150-152 and 187-188, a contiguous portion thereof of at least 14 nucleotides (nt), or a complementary sequence of any of the foregoing; and (i) a target site for TBX21 having the sequence set forth in any one of SEQ ID NOS:153-155, a contiguous portion thereof of at least 14 nucleotides (nt), or a complementary sequence of any of the foregoing.

In any of the embodiments herein, the target site is selected from: (a) a target site for VAV1 having the sequence set forth in any one of SEQ ID NOS:7-9, 156, and 170, or a complementary sequence of any of the foregoing; (b) a target site for IL2 having the sequence set forth in SEQ ID NO:78 or a complementary sequence of any of the foregoing; (c) a target site for BATF having the sequence set forth in any one of SEQ ID NOS:172-174, or a complementary sequence of any of the foregoing; (d) a target site for CD28 having the sequence set forth in any one of SEQ ID NOS:144-146 and 189-191, or a complementary sequence of any of the foregoing; (e) a target site for EOMES having the sequence set forth in any one of SEQ ID NOS:147-149, or a complementary sequence of any of the foregoing; (f) a target site for IRF4 having the sequence set forth in any one of SEQ ID NOS:175-177, or a complementary sequence of any of the foregoing; (g) a target site for LAT having the sequence set forth in any one of SEQ ID NOS:184-186, or a complementary sequence of any of the foregoing; (h) a target site for LCP2 having the sequence set forth in any one of SEQ ID NOS:150-152 and 187-188, or a complementary sequence of any of the foregoing; and (i) a target site for TBX21 having the sequence set forth in any one of SEQ ID NOS:153-155, or a complementary sequence of any of the foregoing.

In any of the embodiments herein, the target site is selected from: (a) a target site for IL-2 having the sequence set forth in SEQ ID NO:78, or a complementary sequence of any of the foregoing; (b) a target site for EOMES having the sequence set forth in SEQ ID NO:149, or a complementary sequence of any of the foregoing; (c) a target site for LCP2 having the sequence set forth in SEQ ID NO:151, or a complementary sequence of any of the foregoing; and (d) a target site for TBX21 having the sequence set forth in SEQ ID NO:155, or a complementary sequence of any of the foregoing.

In any of the embodiments herein, the gRNA is selected from: (a) a gRNA targeting a target site for VAV1 and comprising a gRNA spacer sequence comprising the sequence set forth in any one of SEQ ID NOS:41-43, 169, and 171, or a contiguous portion thereof of at least 14 nt; (b) a gRNA targeting a target site for IL2 and comprising a gRNA spacer sequence comprising the sequence set forth in SEQ ID NO:79, or a contiguous portion thereof of at least 14 nt; (c) a gRNA targeting a target site for BATF and comprising a gRNA spacer sequence comprising the sequence set forth in any one of SEQ ID NOS:178-180, or a contiguous portion thereof of at least 14 nt; (d) a gRNA targeting a target site for CD28 and comprising a gRNA spacer sequence comprising the sequence set forth in any one of SEQ ID NOS:157-159 and 197-199, or a contiguous portion thereof of at least 14 nt; (e) a gRNA targeting a target site for EOMES and comprising a gRNA spacer sequence comprising the sequence set forth in any one of SEQ ID NOS:160-162, or a contiguous portion thereof of at least 14 nt; (f) a gRNA targeting a target site for IRF4 and comprising a gRNA spacer sequence comprising the sequence set forth in any one of SEQ ID NOS:181-183, or a contiguous portion thereof of at least 14 nt; (g) a gRNA targeting a target site for LAT and comprising a gRNA spacer sequence comprising the sequence set forth in any one of SEQ ID NOS:192-194, or a contiguous portion thereof of at least 14 nt; (h) a gRNA targeting a target site for LCP2 and comprising a gRNA spacer sequence comprising the sequence set forth in any one of SEQ ID NOS:163-165 and 195-196, or a contiguous portion thereof of at least 14 nt; and (i) a gRNA targeting a target site for TBX21 and comprising a gRNA spacer sequence comprising the sequence set forth in any one of SEQ ID NOS:166-168, or a contiguous portion thereof of at least 14 nt.

In any of the embodiments herein, the gRNA is selected from: (a) a gRNA targeting a target site for VAV1 and comprising a gRNA spacer sequence set forth in any one of SEQ ID NOS:41-43, 169, and 171; (b) a gRNA targeting a target site for IL2 and comprising a gRNA spacer sequence set forth in SEQ ID NO:79; (c) a gRNA targeting a target site for BATF and comprising a gRNA spacer sequence set forth in any one of SEQ ID NOS:178-180; (d) a gRNA targeting a target site for CD28 and comprising a gRNA spacer sequence set forth in any one of SEQ ID NOS:157-159 and 197-199; (e) a gRNA targeting a target site for EOMES and comprising a gRNA spacer sequence set forth in any one of SEQ ID NOS:160-162; (f) a gRNA targeting a target site for IRF4 and comprising a gRNA spacer sequence set forth in any one of SEQ ID NOS:181-183; (g) a gRNA targeting a target site for LAT and comprising a gRNA spacer sequence set forth in any one of SEQ ID NOS:192-194; (h) a gRNA targeting a target site for LCP2 and comprising a gRNA spacer sequence set forth in any one of SEQ ID NOS:163-165 and 195-196; and (i) a gRNA targeting a target site for TBX21 and comprising a gRNA spacer sequence set forth in any one of SEQ ID NOS:166-168.

In any of the embodiments herein, the gRNA is selected from: (a) a gRNA targeting a target site for IL-2 and comprising a gRNA spacer sequence set forth in SEQ ID NO:79; (b) a gRNA targeting a target site for EOMES and comprising a gRNA spacer sequence set forth in SEQ ID NO:162; (c) a gRNA targeting a target site for LCP2 and comprising a gRNA spacer sequence set forth in SEQ ID NO:164; and (d) a gRNA targeting a target site for TBX21 and comprising a gRNA spacer sequence set forth in SEQ ID NO:168.

In any of the embodiments herein, the gRNA comprises a spacer sequence between 14 nt and 24 nt, or between 16 nt and 22 nt in length. In some embodiments, the gRNA comprises a spacer sequence that is 18 nt, 19 nt, 20 nt, 21 nt, or 22 nt in length.

In any of the embodiments herein, the gRNA further comprises a scaffold sequence set forth in SEQ ID NO:69.

Also provided herein is a combination of gRNAs comprising: two or more gRNAs, each selected from the gRNAs that target a target site for a gene selected from the group consisting of CBLB, CCNC, CD5, CISH, DGKZ, ELOB, FAS, Fli1, GATA3, KDM1A, MED12, MYB, PRDM1, TGFBR2, and RASA2, or two or more gRNAs, each selected from the gRNAs that target a target site for a gene selected from the group consisting of BATF, CD28, EOMES, IL-2, IL2RB, IRF4, LAT, LCP2, TBX21, and VAV1. In some embodiments, the two gRNAs comprise spacer sequences set forth in SEQ ID NOS:92 and 45; SEQ ID NOs: 92 and 62; SEQ ID NOS:45 and 52; and SEQ ID NOS:45 and 53; SEQ ID NOs: 92 and 304; SEQ ID NOs: 92, 304, or 62. In some embodiments, the two gRNAs comprise spacer sequences set forth in SEQ ID NOS: 79 and 164; SEQ ID NOS:79 and 168; SEQ ID NOS:79 and 162.

Also provided herein is a Cas-guide RNA (gRNA) combination comprising: (a) a Clustered Regularly Interspaced Short Palindromic Repeats associated (Cas) protein or variant thereof; and (b) at least one gRNA described herein. In some embodiments, the at least one gRNA targets a target site for a gene selected from the group consisting of CBLB, CCNC, CD5, CISH, DGKZ, ELOB, FAS, Fli1, GATA3, KDM1A, MED12, MYB, PRDM1, TGFBR2, and RASA2.

Also provided herein is a Cas-guide RNA (gRNA) combination comprising: (a) a Clustered Regularly Interspaced Short Palindromic Repeats associated (Cas) protein or variant thereof; and (b) at least one gRNA. In some embodiments, the at least one targets a target site for a gene selected from the group consisting of gRNA BATF, CD28, EOMES, IL-2, IL2RB, IRF4, LAT, LCP2, TBX21, and VAV1.

In any of the embodiments herein, the Cas protein or variant thereof is a deactivated (dCas) protein. In some embodiments, the dCas protein lacks nuclease activity. In some embodiments, the dCas protein is a dCas9 protein. In some embodiments, the dCas protein is a dCas12 protein. In some embodiments, the dCas9 protein is a Staphylococcus aureus dCas9 (dSaCas9) protein. In some embodiments, the dSaCas9 comprises at least one amino acid mutation selected from D10A and N580A, with reference to numbering of positions of SEQ ID NO:124. In some embodiments, the dSaCas9 protein comprises the sequence set forth in SEQ ID NO:125, or an amino acid sequence that has at least 90%, 91%, 92%, 93%, 94%, 95%, 96%, 97%, 98%, or 99% sequence identity thereto. In some embodiments, the dSaCas9 is set forth in SEQ ID NO:125. In some embodiments, the dCas9 protein is a Streptococcus pyogenes dCas9 (dSpCas9) protein. In some embodiments, the dSpCas9 protein comprises at least one amino acid mutation selected from D10A and H840A, with reference to numbering of positions of SEQ ID NO:126. In some embodiments, the dSpCas9 comprises the sequence set forth in SEQ ID NO:127, or an amino acid sequence that has at least 90%, 91%, 92%, 93%, 94%, 95%, 96%, 97%, 98%, or 99% sequence identity thereto. In some embodiments, the dSpCas9 is set forth in SEQ ID NO:127.

Also provided herein is a polynucleotide encoding the epigenetic-modifying DNA-targeting system described herein.

Also provided herein is a polynucleotide encoding at least one DNA-targeting module of the epigenetic-modifying DNA-targeting system described herein.

Also provided herein is a polynucleotide encoding the fusion protein and the at least one gRNA of the epigenetic-modifying DNA-targeting system described herein.

Also provided herein is a polynucleotide encoding the gRNA described herein. In some embodiments, a polynucleotide encodes the combination of gRNAs described herein.

Also provided herein is a polynucleotide encoding the Cas-gRNA combination described herein.

Also provided herein are two or more polynucleotides that together encode: the epigenetic-modifying DNA-targeting system described herein, at least one DNA-targeting module of the epigenetic-modifying DNA-targeting system described herein, the fusion protein and the at least one gRNA of the epigenetic-modifying DNA-targeting system described herein, the combination of gRNAs described herein, and/or the Cas-gRNA combination described herein.

Also provided herein is a polynucleotide gRNA combination comprising: a) a polynucleotide encoding the fusion protein of at least one of the DNA-targeting modules for repressing transcription of the one or more genes of the epigenetic-modifying DNA-targeting system described herein, and one or more gRNAs selected from the gRNA described herein; or b) a polynucleotide encoding the fusion protein of at least one of the DNA-targeting modules for increasing transcription of the one or more genes of the epigenetic-modifying DNA-targeting system described herein, and one or more gRNAs selected from the gRNA described herein. In some embodiments, the polynucleotide encoding the fusion protein is mRNA.

Also provided herein is a vector comprising the polynucleotide described herein.

Also provided herein is a vector comprising the two or more polynucleotides described herein.

Also provided herein is a vector comprising the polynucleotide gRNA combination described herein.

Also provided herein is a vector comprising the polynucleotide gRNA combination described herein.

In any of the embodiments herein, the vector is a viral vector. In some embodiments, the vector is an adeno-associated virus (AAV) vector. In some embodiments, the vector is selected from among AAV1, AAV2, AAV3, AAV4, AAV5, AAV6, AAV7, AAV8, and AAV9.

In any of the embodiments herein, the vector is a non-viral vector. In some embodiments, the non-viral vector is selected from: a lipid nanoparticle, a liposome, an exosome, or a cell penetrating peptide. In some embodiments, the non-viral vector is a lipid nanoparticle.

In any of the embodiments herein, the vector exhibits immune cell tropism, optionally wherein the vector exhibits T-cell tropism.

Also provided herein is a modified T cell comprising the DNA-targeting system described herein, the combination of DNA-targeting systems described herein, the gRNA described herein, the combination of gRNAs described herein, the CRISPR Cas-gRNA combination described herein, the polynucleotide described herein, the two or more polynucleotides described herein, or the polynucleotide gRNA combination described herein.

Also provided herein is a modified T cell comprising an epigenetic or phenotypic modification resulting from being contacted by the DNA-targeting system described herein, the combination of DNA-targeting described herein, the gRNA described herein, the combination of gRNAs described herein, the CRISPR Cas-gRNA combination described herein, the polynucleotide described herein, the two or more polynucleotides described herein, the polynucleotide gRNA combination described herein, or the vector described herein.

In any of the embodiments herein, the modified T cell is derived from a cell from a subject. In any of the embodiments herein, the modified T cell is derived from a primary T cell. In any of the embodiments herein, the modified T cell is derived from a T cell progenitor, a pluripotent stem cell, or an induced pluripotent stem cell. In any of the embodiments herein, the modified T cell further comprises an engineered T cell receptor (eTCR) or chimeric antigen receptor (CAR).

Also provided herein is a method of repressing the transcription of one or more genes in a T cell, the method comprising introducing into a T cell the DNA-targeting system described herein, the combination of DNA-targeting systems described herein, the gRNA described herein, the combination of gRNAs described herein, the Cas-gRNA combination described herein, the polynucleotide described herein, the two or more polynucleotides described herein, the polynucleotide gRNA combination described herein, or the vector described herein. In some embodiments, repressing transcription of the one or more genes promotes increased T cell effector function upon T cell stimulation relative to the T cell effector function in the absence of the T cell stimulation.

Also provided herein is a method of increasing the transcription of one or more genes in a T cell, the method comprising introducing into a T cell the DNA-targeting system described herein, the combination of DNA-targeting systems described herein, the gRNA described herein, the combination of gRNAs described herein, the Cas-gRNA combination described herein, the polynucleotide described herein, the two or more polynucleotides described herein, the polynucleotide gRNA combination described herein, or the vector described herein. In some embodiments, increasing transcription of the one or more genes promotes increased T cell effector function upon T cell stimulation relative to the T cell effector function in the absence of the T cell stimulation.

Also provided herein is a method of increasing T cell effector function, the method comprising introducing into a T cell the DNA-targeting system described herein, the combination of DNA-targeting systems described herein, the gRNA described herein, the combination described herein, the CRISPR Cas-gRNA combination described herein, the polynucleotide described herein, the two or more polynucleotides described herein, the polynucleotide gRNA combination described herein, or the vector described herein.

In any of the embodiments herein, the T cell effector function is increased compared to a T cell that has not been introduced with the DNA-targeting system described herein, the combination of DNA-targeting systems described herein, the gRNA described herein, the combination of gRNAs described herein, the CRISPR Cas-gRNA combination described herein, the polynucleotide described herein, the two or more polynucleotides described herein, the polynucleotide gRNA combination described herein, or the vector described herein.

In any of the embodiments herein, the T cell is a T cell in a subject and the method is carried out in vivo. In any of the embodiments herein, the T cell is a T cell from a subject, or derived from a cell from the subject, and the method is carried out ex vivo. In any of the embodiments herein, the T cell is a primary T cell. In some embodiments herein, the T cell is derived from a T cell progenitor, a pluripotent stem cell, or an induced pluripotent stem cell.

In any of the embodiments herein, the introducing is by transient delivery into the T cell. In some embodiments, the transient delivery comprises electroporation, transfection, or transduction.

In any of the embodiments herein, the DNA-targeting system of any of claims 1-95, the combination of DNA-targeting systems described herein, the gRNA described herein, the combination of gRNAs described herein, the CRISPR Cas-gRNA described herein, the polynucleotide described herein, the two or more polynucleotides described herein, the polynucleotide gRNA combination described herein, or the vector described herein, is transiently expressed and/or transiently present in the T cell.

In any of the embodiments herein, the introducing represses transcription of one or more genes in the T cell selected from the group consisting of CBLB, CCNC, CD5, CISH, DGKZ, ELOB, FAS, Fli1, GATA3, KDM1A, MED12, MYB, PRDM1, TGFBR2, and RASA2.

In any of the embodiments herein, the introducing increases transcription of one or more genes in the T cells selected from the group consisting of BATF, CD28, EOMES, IL-2, IL2RB, IRF4, LAT, LCP2, TBX21, and VAV1.

Also provided herein is a modified T cell produced by the method described herein.

Also provided herein is a method of treating a disease or condition in a subject, the method comprising administering to the subject the modified T cell described herein.

Also provided herein is a method of increasing T cell persistence in T cells of a subject, the method comprising administering to the subject or T cells thereof the DNA-targeting system described herein, the combination of DNA-targeting systems described herein, the gRNA described herein, the combination of gRNAs described herein, the CRISPR Cas-gRNA combination described herein, the polynucleotide described herein, the two or more polynucleotides described herein, the polynucleotide gRNA combination described herein, or the vector described herein. In some embodiments, the T cell is from an adoptive T cell therapy for treating a disease or condition in the subject. In some embodiments, the T cell therapy comprises T cells expressing a recombinant receptor specific for a target antigen. In some embodiments, the administration is carried out prior to, concurrently with, or after administration of the adoptive T cell therapy. In some embodiments, the administration is carried out after administration of the adoptive T cell therapy in the subject, at a time after the numbers or effector function of T cells of the adoptive T cell therapy are reduced, or are suspected of being reduced, in the subject.

Also provided herein is a method of treating a disease or condition in a subject, the method comprising administering to a subject: a T cell therapy comprising cells expressing a recombinant receptor specific for a target antigen associated with the disease or condition; and the DNA-targeting system described herein, the combination of DNA-targeting systems described herein, the gRNA described herein, the combination of gRNAs described herein, the CRISPR Cas-gRNA combination described herein, the polynucleotide described herein, the two or more polynucleotides described herein, the polynucleotide gRNA combination described herein, or the vector described herein. In some embodiments, the recombinant receptor is an engineered T cell receptor (eTCR) or chimeric antigen receptor (CAR).

In any of the embodiments herein, the target antigen is a tumor antigen. In some embodiments, the disease or condition is a cancer. In some embodiments, the cancer is a hematological cancer or is a solid tumor. In any of the embodiments herein, the disease or condition is an autoimmune condition and/or an inflammatory condition.

In any of the embodiments herein, the administering results in transient delivery into the T cell: the DNA-targeting system, the combination of DNA-targeting systems, the gRNA, the combination of gRNAs, the CRISPR Cas-gRNA combination, the polynucleotide, the two or more polynucleotides, the polynucleotide gRNA combination, or the vector.

In any of the embodiments herein, the administering represses transcription of one or more genes in the T cell selected from the group consisting of CBLB, CCNC, CD5, CISH, DGKZ, ELOB, FAS, Fli1, GATA3, KDM1A, MED12, MYB, PRDM1, TGFBR2, and RASA2.

In any of the embodiments herein, the administering represses transcription of one or more genes in the T cell selected from the group consisting of CBLB, CISH, MED12, MYB, PRDM1, and RASA2.

In any of the embodiments herein, the administering increases transcription of one or more genes in the T cells selected from the group consisting of BATF, CD28, EOMES, IL-2, IL2RB, IRF4, LAT, LCP2, TBX21, and VAV1.

In any of the embodiments herein, the administering increases transcription of one or more genes in the T cells selected from the group consisting of EOMES, IL-2, LCP2, and TBX21.

### BRIEF DESCRIPTION OF THE DRAWINGS

FIG. 1A shows an exemplary workflow for a transient CRISPR screen. Cells are transfected with a gRNA library. Following enrichment of transfected cells, an epi-editor, such as a dCas fused to an effector domain (e.g., transcriptional repressor, transcriptional activator, etc.), is transiently transfected into the cells, such as by electroporation. Cells are then screened for the desired phenotype. FIG. 1B shows an exemplary plot showing expression of IL-2 and IFNg in cells as assessed by flow cytometry, with boxes indicating populations to be sorted according to different phenotypes.

FIG. 2 shows a plot of an exemplary CRISPRi screen for gRNAs and genes that modulate IL-2 expression, IFNg expression, and/or proliferation. In particular, the plot shows gRNAs affecting IL-2 expression. Dots represent individual gRNAs. gRNAs on the left are those that target genes whose inhibition results in decreased IL-2 expression, while gRNAs on the right are those that target genes whose inhibition result in upregulation of IL-2 expression. Dots represent individual gRNAs. X-axis represents log 2 fold change of gRNA abundance in IL-2+ sorted cells versus unsorted cells. Y-axis represents significance (−log 10 adjusted p-value).

FIG. 3 shows a plot from an exemplary CRISPRi screen for gRNAs and genes that modulate IL-2 expression, IFNg expression, and/or proliferation. In particular, the plot shows gRNAs affecting proliferation. Dots represent individual gRNAs. gRNAs on the left are those that target genes whose inhibition results in decreased proliferation, while gRNAs on the right are those that target genes whose inhibition result in increased proliferation. X-axis represents log 2 fold change of gRNA abundance in unsorted cells 6 days after electroporation versus unsorted cells before electroporation with a transiently expressed epi-editor for targeted transcriptional repression. Y-axis represents significance (−log 10 adjusted p-value).

FIG. 4 shows numbers of gRNA hits from indicated conditions at day 9, day 12, or both day 9 and day 12 of a CRISPRi screen for gRNAs and genes modulating T cell phenotypes.

FIG. 5 shows a plot from an exemplary CRISPRa screen for gRNAs and genes that modulate IL-2 expression, IFNg expression, and/or proliferation. In particular, the plot shows gRNAs affecting IL-2 expression. Dots represent individual gRNAs. gRNAs on the left are those that target genes whose activation results in decreased IL-2 expression, while gRNAs on the right are those that target genes whose activation results in increased IL-2 expression. X-axis represents log 2 fold change of gRNA abundance in IL-2+ sorted cells versus unsorted cells. Y-axis represents significance (−log 10 adjusted p-value).

FIG. 6A shows percent knockdown (% KD) of MED12 expression, as assessed by RT-qPCR following transient delivery of dSpCas9-KRAB and indicated MED12-targeting gRNAs or non-targeting gRNAs (NT1, NT2), or dSpCas9-KRAB alone. Results are shown for 48 hours and 6 days after transfection, and for T cells from two different donors.

FIG. 6B shows MED12 expression, as assessed by RT-qPCR following transient delivery of dSpCas9-KRAB and indicated MED12-targeting gRNAs in T cells (left) and CAR T cells (right). Control conditions for left panel include cells delivered with dSpCas9-KRAB and non-targeting gRNAs (NT1, NT2), and dSpCas9-KRAB alone. Control conditions for right panel include cells not expressing a CAR (Mock), and cells not delivered with a DNA-targeting system. Results are shown for 48 hours (left) and 72 hours and 7 days after transfection. Results are normalized to dSpCas9-KRAB only condition (left panel) or Mock condition with (right panel).

FIG. 6C shows percent knockdown (% KD) of CCNC expression, as assessed by RT-qPCR following transient delivery of dSpCas9-KRAB and indicated CCNC-targeting gRNAs or non-targeting gRNAs (NT1, NT2), or dSpCas9-KRAB alone. Results are shown for 48 hours and 6 days after transfection, and for T cells from two different donors.

FIG. 6D shows percent knockdown (% KD) of FAS expression, as assessed by intracellular cytokine staining (ICS) and flow cytometry following transient delivery of dSpCas9-KRAB and indicated FAS-targeting gRNAs, dSpCas9-KRAB alone, or mock delivery. Results are shown for 72 hours and 7 days after delivery, and for T cells from two different donors. Top panel shows an exemplary flow cytometry plot for assessing FAS expression in CD3+ T cells following delivery with dSpCas9-KRAB only, or dSpCas9-KRAB and a FAS-targeting gRNA.

FIG. 7A shows flow cytometry plots for assessing IL-2 expression in CD3+ Her2 CAR T cells after stimulation and transient delivery of dSpCas9-2xVP64 and an IL-2-targeting gRNA (IL-21, targeting SEQ ID NO:78). Results are shown for CAR T cells derived from 2 different donors. CAR T cells were stimulated with SKOV3 or 143B cells. Different control conditions excluded the CAR, the gRNA, or stimulation, as indicated.

FIG. 7B shows quantified IL-2 expression (% IL-2+ cells as assessed by flow cytometry) in Her2 CAR T cells after stimulation and transient delivery of dSpCas9-2xVP64 and an IL-2-targeting gRNA (IL-2_1, targeting SEQ ID NO:78). Control conditions excluded the CAR (mock transduction), the gRNA, or stimulation, as indicated.

FIG. 7C shows IL-2 expression in CAR T cells following delivery of a DNA-targeting system for IL-2 activation. Left panel shows IL-2 expression in Her2 CAR T cells as assessed RT-qPCR at 72 hours and 7 days following transient delivery with a DNA-targeting system containing dSpCas9-2xVP64 and an IL-2 targeting gRNA (IL-2_1, targeting SEQ ID NO:78). Control conditions included cells not expressing the CAR (mock), or CAR T cells not delivered with a DNA-targeting system (CAR Only). Expression levels were normalized to the mock control cells. Right panel shows IL-2 expression in control cells (CAR only) and CAR T cells delivered with the DNA-targeting system for IL-2 activation following stimulation with Her2 antigen-expressing tumor cells, as assessed by ICS and flow cytometry.

FIGS. 8A-8C show flow cytometry plots for assessing IL-2 (FIG. 8A), IFNg (FIG. 8B), or TNFa (FIG. 8C) expression in CD3+ Her2 CAR T cells after stimulation and transient delivery of dSpCas9-2xVP64 and a VAV1-targeting gRNA (VAV1_5, targeting SEQ ID NO:170). Results are shown for CAR T cells derived from 2 different donors. CAR T cells were stimulated with SKOV3 or 143B cells. Different control conditions excluded the CAR, the gRNA, or stimulation, as indicated.

FIG. 9A shows percentage polyfunctional cells (IL-2+/IFNg+/TNFa+ cells) as assessed by flow cytometry in Her2 CAR T cells after stimulation and transient delivery of dSpCas9-2xVP64 and an IL-2-targeting gRNA (IL-21, targeting SEQ ID NO:78) or a VAV1-targeting gRNA (VAV1_5, targeting SEQ ID NO:170). Control conditions excluded the CAR (mock transduction), the gRNA, or stimulation, as indicated.

FIG. 9B shows percentage of IL-2 expressing Her2 CAR T cells over a period of days after stimulation and transient delivery of dSpCas9-2xVP64 and an IL-2-targeting gRNA (IL-2_1, targeting SEQ ID NO:78) or a VAV1-targeting gRNA (VAV1_5, targeting SEQ ID NO:170). FIG. 9C shows flow cytometry plots for assessing IL-2 expression in Her2 CAR T cells before and after stimulation and transient delivery of gRNA and effector systems.

FIG. 10A-10B shows flow cytometry plots and mean fluorescence intensity (MFI) for assessing IL-2 and IFNg expression in CD4+(FIG. 10A) or CD8+(FIG. 10B) Her2 CAR T cells after stimulation and transient delivery of dSpCas9-2xVP64 and indicated CBLB-targeting gRNAs or a control non-targeting gRNA. Flow cytometry plots allow for quantification of percentage of cells with specific phenotypes (e.g. IL-2+), and for quantification of mean fluorescence intensity (MFI; corresponding to average expression levels), as shown in bottom panels.

FIG. 11A shows a heatmap indicating relative levels of intracellular cytokine expression (as assessed by ICS and flow cytometry) in Her2 CAR T cells after stimulation and transient delivery of dSpCas9-2xVP64 and indicated gRNAs. Cytokine expression was quantified according to % Her2 CAR T cells with the indicated phenotypes (e.g. CD4+/IL-2+, CD4+/IFNg+/TNFa+) as assessed by flow cytometry, and log 2 fold change was calculated with respect to the control condition with a non-targeting gRNA (non-targeting_sp_1). Darker shades correspond to higher cytokine expression, as indicated in the legend. Each condition was assigned a cumulative score according to all cytokine expression measurements and ranked from low to high cytokine expression.

FIG. 11B shows exemplary results from a serial stimulation killing assay. Growth of antigen-expressing target cells expressing a fluorescent marker was quantified over time based on overall fluorescence using an Incucyte automated tracking system. Antigen-expressing target cells were co-cultured with CAR T cells transiently delivered with a DNA-targeting system for activation of IL-2, CAR T cells transiently delivered with a DNA-targeting system for repression of MED12, CAR T cells with no DNA-targeting system (CAR alone), or cells not expressing a CAR (Mock). Serial stimulations (shown as stim 1, stim 2, and stim 3) were performed by replating CAR T cells with fresh target cells at a 1:4 ratio of CAR T cells:target cells.

FIG. 11C shows quantification of fold expansion (left) and IL-2 secretion (right) by CAR T cells and control cells from experiment shown in FIG. 11B, following the second stimulation.

FIG. 12A depicts flow cytometry plots of intracellular cytokine staining (ICS) for IL-2 and IFN-g expression after transient transfection of T cells with control dSpCas9-2xVP64 effector only (without targeting gRNA) or with dSpCas9-2xVP64 in combination with a gRNA targeting either VAV1 (VAV1_5) or IL-2 (IL-2_1) individually.

FIG. 12B depicts an ICS flow cytometry plot for IL-2 and IFN-g after transient transfection of T cells with dSpCas9-2xVP64 effector and gRNAs targeting both VAV1 and IL-2.

FIG. 12C depicts the percent of IL-2+ cells (left panel) or IL-2+, IFNg+ and TNFalpha+ cells (right panel) after transient transfection of T cells from either of two donors with dSpCas9-2xVP64 effector only (without targeting gRNA), dSpCas9-2xVP64 in combination with VAV1 or IL-2, or dSpCas9-2xVP64 in combination with gRNAs targeting both VAV1 and IL-2.

FIGS. 13A-13B show heatmaps indicating relative levels of intracellular cytokine expression, cytokine secretion, proliferation, and target cell killing activity in Her2 CAR T cells after stimulation and transient delivery of dSpCas9-2xVP64 or dSpCas9-KRAB and the indicated gRNAs, for Her2 CAR T cells derived from a first donor (FIG. 13A) and a second donor (FIG. 13B). Multiple rounds of stimulation were performed, and cells electroporated with the different DNA-targeting systems were assessed based on multiple readouts of T cell effector function after the stimulations, including intracellular cytokine expression (by ICS), cytokine secretion, proliferation, and killing of target cells, as described in Example 5. The readouts of T cell effector function were quantified after a first, second, and/or third stimulation (shown as stim 1, stim 2, or stim 3 in figures). ICS was performed after a first stimulation only. Results are shown as Log 2 fold-change in comparison to control cells delivered with a non-targeting gRNA (NT). Darker shades correspond to increased measured T cell effector functions, as indicated in the legend. Each condition was assigned a cumulative score according to all measurements and ranked from low to high T cell effector function.

FIG. 14 shows a heatmap indicating relative levels of intracellular cytokine expression in Her2 CAR T cells after stimulation and transient delivery of dSpCas9-2xVP64 and a combination of 2 gRNAs targeting the indicated genes. gRNAs for targeting each gene are shown next to the heatmap. Where the genes targeted are the same, the same gRNA was delivered at twice the concentration. Cytokine expression was quantified according to % Her2 CAR T cells with the indicated phenotypes (e.g. CD8+/IL-2+/IFNg+/TNFa+) as assessed by ICS and flow cytometry, and log 2 fold change was calculated with respect to the control condition with a non-targeting gRNAs (NT+NT). Darker shades correspond to higher cytokine expression, as indicated in the legend. Each condition was assigned a cumulative score according to all cytokine expression measurements and ranked from low to high cytokine expression.

FIGS. 15A-15B show heatmaps indicating relative levels of proliferation and cytokine expression in Her2 CAR T cells after stimulation and transient delivery of dSpCas9-2xVP64 or dSpCas9-KRAB and gRNAs or combinations thereof targeting the indicated genes. gRNAs for targeting each gene are indicated in the accompanying tables. Cytokine expression was quantified according to % Her2 CAR T cells with the indicated phenotypes (e.g. CD4+/IL-2+/IFNg+/TNFa+) as assessed by ICS and flow cytometry. Proliferation was quantified according to fold change of Her2 CAR T cell numbers before and after 30timulateon, as described herein. For each cytokine or proliferation phenotype value, log 2 fold change was calculated with respect to a control condition with a non-targeting gRNA (non-targeting_sp_1 for FIG. 15A; dCas only for FIG. 15B). Darker shades correspond to increased proliferation and cytokine expression, as indicated in the legend. Each condition was assigned a cumulative score according to all cytokine and proliferation measurements and ranked from low to high cytokine expression and proliferation.

FIG. 16 shows fold expansion (i.e. proliferation) of Her2 CAR T cells after stimulation and transient delivery of dSpCas9-2xVP64 or dSpCas9-KRAB and indicated gRNAs. Results are shown for Her2 CAR T cells derived from two different donors, after a first stimulation with antigen-expressing target cells. Negative control conditions included CAR T cells delivered with a dCas-effector and no gRNA, CAR T cells not delivered with a DNA-targeting system (CAR only), and cells not expressing a CAR (mock).

FIG. 17 shows levels of secreted cytokines IL-2 or IFNg in Her2 CAR T cells after stimulation and transient delivery of dSpCas9-2xVP64 or dSpCas9-KRAB and indicated gRNAs. Results are shown for Her2 CAR T cells derived from two different donors, after a first stimulation with antigen-expresing target cells. Negative control conditions included CAR T cells delivered with a dCas-effector and no gRNA, CAR T cells not delivered with a DNA-targeting system (CAR only), and cells not expressing a CAR (mock).

FIG. 18 shows killing activity based on calculated killing index in Her2 CAR T cells after transient delivery of dSpCas9-2xVP64 or dSpCas9-KRAB and indicated gRNAs, after a third stimulation with antigen-expressing target cells. Negative control conditions included CAR T cells delivered with a dCas-effector and no gRNA, CAR T cells not delivered with a DNA-targeting system (CAR only), and cells not expressing a CAR (mock).

FIGS. 19A-19B show heatmaps indicating relative levels of quantified T cell effector functions in Her2 CAR T cells after stimulation and transient delivery of dSpCas9-2xVP64 or dSpCas9-KRAB and indicated gRNAs. Results are shown for cells derived from a first donor (FIG. 19A) and second donor (FIG. 19B). Assessed T cell effector functions included intracellular cytokine expression, secreted cytokine expression, proliferation, and killing, which were measured as described in Example 5. The T cell effector functions were quantified after a first, second, and/or third stimulation with antigen-expressing target cells, as indicated in the figure. For each quantified T cell effector function, results are shown as log 2 fold change as calculated with respect to the negative control condition (CAR alone, i.e. CAR T cells not delivered with a DNA-targeting system). Darker shades correspond to increased measured T cell effector functions, as indicated in the legend. Each condition was assigned a cumulative score according to all measurements and ranked from low to high T cell effector function.

FIGS. 20A-20C show results from CAR T cells electroporated with gRNAs targeting indicated genes and mRNA encoding either dSpCas9-2xVP64 for activation or dSpCas9-KRAB for repression. For all results, experiments were performed using CAR T cells from two different donors, and results are plotted for each of the two donors (triangles), with average value indicated by vertical line. FIG. 20A shows CAR T fold expansion following electroporation of the DNA-targeting systems (production), and following a first, second, and third round of stimulation with Her2 antigen-expressing target cells. FIG. 20B shows CAR T cell target cell killing index following the first and second round of stimulation. FIG. 20C shows CAR T cell cytokine production as assessed by ICS and flow cytometry at Day 9 post-electroporation with the DNA-targeting systems, and after a first stimulation with Her2 antigen-expressing target cells.

FIGS. 21A-21C show results from stimulated CAR T cells delivered with DNA-targeting systems for repression of TGF-beta receptor 2 (TGFBR2). DNA-targeting systems included indicated TGFBR2-targeting gRNAs, and either dSpCas9-KRAB (SEQ ID NO:332) or DNMT3A/L-XTEN80-dSpCas9-KRAB (SEQ ID NO:337). Control cells included dSpCas9 only, cells not expressing a CAR (Mock), or CAR T cells not stimulated with antigen-expressing cells (CAR Alone). FIG. 21A shows expression of TGFBR2 at 48 hours post-electroporation with the DNA-targeting systems. FIG. 21B shows secreted IFNg from the CAR T cells 24 hours after a second stimulation with Her2 antigen-expressing cells, in the presence of 10 ng/mL TGFb. FIG. 21C shows fold expansion of the stimulated CAR T cells after the second stimulation with Her2 antigen-expressing cells in the presence of 10 ng/mL TGFb.

FIGS. 21D-21G show results from stimulated CAR T cells delivered with DNA-targeting systems for repression of TGF-beta receptor 2 (TGFBR2). CAR T cells were electroporated for transient expression of DNA-targeting systems composed of dSpCas9-KRAB-DNMT3A/L and indicated gRNAs targeting TGFBR2. Negative control cells were electroporated with dSpCas9-KRAB-DNMT3A/L and a non-targeting gRNA (non-targeting), not electroporated with a DNA-targeting system (CAR only), or did not express a CAR (mock). FIG. 21D shows % TGFBR2 negative cells after indicated days post-electroporation. FIG. 21E shows fold cell expansion of CAR T cells after stimulation with anti-CD3/anti-CD28 coated wells and exposure to indicated concentrations of TGF-beta, with expansion normalized to conditions with 0 ng/mL TGF-beta. FIG. 21F shows production of IFNg by CAR T cells in response to exposure to 10 ng/mL TGF-beta. Dotted horizontal line indicates CAR only control condition. FIG. 21G shows levels of secreted cytokines in stimulated CAR T cells after exposure to indicated concentrations of TGF-beta, with secreted cytokine levels normalized to conditions with 0 ng/mL.

FIGS. 22A-22D show time course (FIG. 22A) and results (FIG. 22B-FIG. 22Dfrom experiment for in vivo assessment of CAR T cells delivered with a DNA-targeting system for IL-2 activation, or for MED12 or CBLB repression. The in vivo experiments were carried out in immune-deficient mice transplanted implanted with antigen-expressing tumor cells (NCI-H1975) and injected with CAR T cells. FIG. 22A shows timing and details of tumor implantation, CAR T cell injection, and tracking. FIG. 22B shows results from experiments, including animal survival (left panels), tumor growth (middle panels), and levels of circulating CAR T cells (right panels) for CAR T cells delivered with the DNA-targeting system for IL-2 activation (dSpCas9-2xVP64 and gRNA IL-2_1, targeting SEQ ID NO:78). FIG. 22C shows results from experiments, including animal survival (left panels), tumor growth (middle panels), and levels of circulating CAR T cells (right panels) for CAR T cells delivered with the DNA-targeting system for MED12 repression (dSpCas9-KRAB and gRNA MED12_2, targeting SEQ ID NO:81). FIG. 22D shows results from experiments, including tumor growth (left panel) and levels of circulating CAR T cells (right panel) for CAR T cells delivered with the DNA-targeting system for for CBLB repression (dSpCas9-KRAB and gRNA CBLB_2, targeting SEQ ID NO:11). Control mice were injected with CAR T cells not delivered with a DNA-targeting system (CAR alone), were injected with T cells not expressing a CAR (Mock T Cells), or were not injected with T cells (Tumor Alone).

FIG. 23 shows MED12 expression as assessed by RT-qPCR at day 4 and day 21 post-electroporation with a MED12-targeting gRNA (MED12_2, targeting SEQ ID NO:81) and mRNA encoding dSpCas9 (control; no transcriptional repressor effector domain), dSpCas9-KRAB (SEQ ID NO:332), or DNMT3A/L-XTEN80-dSpCas9-KRAB (SEQ ID NO: 337). Expression levels (shown as fold-change in expression) are normalized to expression levels in T cells electroporated with the same fusion protein but with a non-targeting gRNA (dotted line at 1.0).

FIGS. 24A-24F show results from CAR T cells delivered with DNA-targeting systems comprising indicated MED12-targeting gRNAs and dSpCas9-KRAB (SEQ ID NO:332), or DNMT3A/L-XTEN80-dSpCas9-KRAB (SEQ ID NO: 337). Control cells included cells delivered with DNMT3A/L-XTEN80-dSpCas9-KRAB and no gRNA, DNMT3A/L-XTEN80-dSpCas9-KRAB and a non-targeting gRNA, and T cells not expressing a CAR (Mock). FIG. 24A shows MED12 expression as assessed by qRT-PCR at day 10 post-electroporation with the DNA-targeting systems. FIG. 24B shows MED12 expression at days 2, 7, 10, and 14 post-electroporation. FIG. 24C shows CD25 expression as assessed by flow cytometry in CAR T cells at days 3, 7, 10, and 14 post-electroporation. FIG. 24D shows secreted IFN-gamma expression, and FIG. 24E shows secreted IL-2 expression, at 24 hours after a second stimulation of the CAR T cells with Her2-positive NCI-H1975 tumor cells. FIG. 24F shows proliferation (fold expansion normalized to fold expansion of CAR alone controls) after the second stimulation with the antigen-expressing cells.

FIG. 25 shows a timecourse of expression of an exemplary dSpCas9 protein following electroporation of mRNA encoding the protein. Results are shown for electroporation of the dSpCas9 protein with a non-targeting gRNA, a gRNA targeting a gene in the cell, or no electroporation (control). Expression was assessed based on an associated GFP tag, with results indicating percent GFP+ cells as assessed by flow cytometry at indicated time points.

FIGS. 26A-26B show arrangements of fusion proteins for targeted transcriptional repression (FIG. 26A), and results from experiments testing ability of the fusion proteins to mediate targeted and sustained gene repression (FIG. 26B). FIG. 26A shows 4 different arrangements of the dSpCas9 fusion proteins from N-terminal to C-terminal, with the fusion proteins including various domains selected from a DNMT3A, DNMT3B, DNMT3L, and a KRAB or EZH2 domain (shown as [KRAB]). FIG. 26B shows MED12 expression at 4 days and 21 days in T cells electroporated with a gRNA targeting MED12 (MED12_2, targeting SEQ ID NO:81), and mRNA encoding the fusion proteins having each of the 4 different arrangements, and comprising a repression domain (shown as [KRAB]) selected from: a KRAB domain from KOX1 (KOX1(2-99)) (SEQ ID NO:355; KRAB domain used in dSpCas9 fusion proteins of preceding Examples), a KRAB domain from KOX1 (KOX1(1-72)) (SEQ ID NO:356), a KRAB domain from ZIM3 (SEQ ID NO:357), a KRAB domain from ZNF324 (SEQ ID NO:358), and an EZH2 domain (SEQ ID NO:359). The mRNA encoding the fusion proteins further included an N-terminal FLAG epitope and a C-terminal P2A-mCherry domain to assess expression of the fusion protein. For each experimental condition, MED 12 expression was normalized to expression levels in T cells electroporated with the same fusion protein but with a non-targeting gRNA.

FIG. 27A shows expression of indicated genes as assessed by RT-qPCR in CAR T cells 72 hours after transient delivery of a dSpCas9 fusion protein for repression (e.g. dSpCas9-KRAB-DNMT3A/L) and gRNAs targeting the indicated genes and/or a control non-targeting gRNA (“NT guide”).

FIG. 27B shows IL-2 expression measured by ICS and flow cytometry. IL-2 expression was determined based on the percentage of viable T cells that were IL-2+ after each indicated stimulation, and quantified as fold-change over control cells delivered with a non-targeting gRNA.

FIG. 27C shows IL-2 expression before and after TGF-beta treatment and transient delivery of the DNA-targeting systems for multiplexed repression of MED12 and TGFBR2.

FIGS. 28A-28B show tumor cell killing over time by Her2 CAR T cells that were delivered mRNA encoding either an dSpCas9-2xVP64 effector fusion protein, an SpCas9 IL-2-targeting gRNA, and an SpCas9 TBX21-targeting gRNA or a dSpCas9-KRAB-DNMT3A/L effector fusion protein, an SpCas9 MED12-targeting gRNA, and an SpCas9 CBLB-targeting gRNA.

FIG. 29A and FIG. 29B show results from experiments in an mouse tumor model for in vivo assessment of CAR T cells that had been transiently delivered with single or multiplexed DNA-targeting systems for IL-2, LCP2, EOMES, or TBX21 activation, in which CAR T cells were administered to miceat low dose (FIG. 29A) or high dose (FIG. 29B).

FIG. 30A and FIG. 30B show results from experiments in a mouse tumor model for in vivo assessment of CAR T cells that had been transiently delivered with single or multiplexed DNA-targeting systems for MED12, CBLB and/or or CISH repression, in which CAR T cells were administered to mice at low dose (FIG. 30A) or high dose (FIG. 30B).

### DETAILED DESCRIPTION

Provided herein is an epigenetic-modifying DNA-targeting system in which the DNA-targeting system comprises at least one DNA-targeting module composed of a fusion protein comprising: (a) a DNA-binding domain capable of being targeted to a target site in one or more genes or regulatory DNA element thereof in a T cell; and (b) at least one effector domain capable of modulating transcription of the one or more genes. In some embodiments, the target site is in a gene or regulatory region thereof found herein to be a negative regulator of T cell function after transient transcriptional modulation of the gene, such as a target site in CBLB, CCNC, CD5, CISH, DGKZ, ELOB, FAS, Fli1, GATA3, KDM1A, MED12, MYB, PRDM1, TGFBR2, and/or RASA2. In some such embodiments, the at least one effector domain is a transcriptional repressor domain, such as KRAB or DNMT3A/3L or combinations thereof. In some embodiments, the target site is in a gene or regulatory region thereof found herein to be a positive regulator of T cell function after transient transcriptional modulation of the gene, such as a target site in BATF, CD28, EOMES, IL-2, IL2RB, IRF4, LAT, LCP2, TBX21, and/or VAV1. In some such embodiments, the at least one effector domain is a transcriptional activator domain, such as VP64. In some embodiments, the target site is within 1000 base pairs of a transcriptional start site (TSS) of any such genes, for example, the target site may be within a regulatory region, such as a promoter or enhancer of any such genes.

In some embodiments, the DNA-targeting systems are synthetic transcription factors that are able to modulate, such as decrease (or downregulate) or increase (or upregulate), transcription of a gene in a targeted manner. In some embodiments, the DNA-binding domain of the DNA-targeting system is a nuclease-inactive Clustered Regularly Interspaced Short Palindromic Repeats associated (Cas) protein (e.g., a dCas protein) or variant thereof complexed with a guide RNA (gRNA). Also provided are gRNAs for targeting to a target site in a gene or a regulatory DNA element thereof in a T cell, wherein the gene is any as provided herein in which it is found that transient epigenetic modulation of transcription of the gene promotes T cell function. Also provided are CRISPR-Cas/gRNA combinations thereof composed of the gRNA and a nuclease inactivated Cas, such as a dCas9. Also provided herein are polynucleotides encoding the DNA-targeting system or the fusion protein of the DNA-targeting system, and vectors and cells containing the same. Also provided herein are methods of using the epigenetic-modifying DNA-targeting system for modulating transcription or phenotype or function of T cells and the resulting modified cells.

In some embodiments, the DNA-targeting system contains at least one DNA-targeting module, where each DNA-targeting module of the system is a component of the DNA-targeting system that is independently capable of targeting one target site for a target gene as provided. In some embodiments, each DNA-targeting module includes (a) a DNA-binding domain capable of being targeted to a target site for a provided target gene and (b) an effector domain capable of modulating (e.g. repressing or activating) transcription of the gene.

In some embodiments, the DNA-targeting system includes a single DNA-targeting module for targeting repression of a single gene. In some embodiments, the gene is CBLB, CCNC, CD5, CISH, DGKZ, ELOB, FAS, Fli1, GATA3, KDM1A, MED12, MYB, PRDM1, TGFBR2, or RASA2. In some embodiments, the gene is CBLB, CISH, MED12, MYB, PRDM1, or RASA2. In some embodiments, the DNA-targeting module includes (a) a DNA-binding domain capable of being targeted to a target site of the target gene or regulatory element, and (b) an effector domain capable of reducing transcription of the gene.

In some embodiments, the DNA-targeting system includes a plurality of DNA-targeting modules, in which each DNA-targeting module is for targeting repression of a different gene. In some embodiments, the DNA-targeting systems are multiplexed DNA-targeting systems, i.e. targeted to target sites for more than one gene. Hence, the terms DNA-targeting system may include a multiplexed epigenetic-modifying DNA targeting system that includes more than one DNA-targeting module. A multiplexed epigenetic-modifying DNA targeting system comprises at least 2, at least 3, at least 4, at least 5, at least 6, at least 7, at least 8, at least 9, at least 10, at least 11, at least 12 DNA-targeting modules. In some embodiments, the plurality of DNA-targeting modules target a plurality of target sites for one or more genes, such as 2, 3, 4 or more genes, selected from CBLB, CCNC, CD5, CISH, DGKZ, ELOB, FAS, Fli1, GATA3, KDM1A, MED12, MYB, PRDM1, TGFBR2, and RASA2.

In some embodiments, the DNA-targeting system includes a single DNA-targeting module for targeting activation or increased expression of a single gene. In some embodiments, the gene is BATF, CD28, EOMES, IL-2, IL2RB, IRF4, LAT, LCP2, TBX21, or VAV1. In some embodiments, the gene is EOMES, IL-2, LCP2, or TBX21. In some embodiments, the DNA-targeting module includes (a) a DNA-binding domain capable of being targeted to a target site of the target gene or regulatory element, and (b) an effector domain capable of activating transcription of the gene.

In some embodiments, the DNA-targeting system includes a plurality of DNA-targeting modules, in which each DNA-targeting module is for targeting activation or increased expression of a different gene. In some embodiments, the DNA-targeting systems are multiplexed DNA-targeting systems, i.e. targeted to target sites for more than one gene. Hence, the terms DNA-targeting system may include a multiplexed epigenetic-modifying DNA targeting system that includes more than one DNA-targeting module. A multiplexed epigenetic-modifying DNA targeting system comprises at least 2, at least 3, at least 4, at least 5, at least 6, at least 7, at least 8, at least 9, at least 10, at least 11, at least 12 DNA-targeting modules. In some embodiments, the plurality of DNA-targeting modules target a plurality of target sites for one or more genes, such as 2 or 3 genes, selected from BATF, CD28, EOMES, IL-2, IL2RB, IRF4, LAT, LCP2, TBX21, and VAV1.

In some embodiments, any two DNA-targeting modules of a DNA-targeting system comprise separate (i.e. non-overlapping) components. In some embodiments, different DNA-targeting modules of a DNA-targeting system comprise separate (i.e. non-overlapping) components. For example, a DNA-targeting system may comprise a first DNA-targeting module comprising a first fusion protein comprising a DNA-binding domain (e.g. a ZFN or TALE-based DNA-binding domain) that targets a first target site, and a second DNA-targeting module comprising a second fusion protein comprising a second DNA-binding domain (e.g. a ZFN or TALE-based DNA-binding domain) that targets a second target site.

In some embodiments, any two DNA-targeting modules of a DNA-targeting system may comprise shared (i.e. overlapping) components. In some embodiments, different DNA-targeting modules of a DNA-targeting system comprise shared (i.e. overlapping) components. For example, in one aspect, a DNA-targeting system may comprise a first DNA-targeting module comprising (a) a fusion protein comprising a Cas protein and a transcriptional effector (e.g. repressor) domain, and (b) a first gRNA that complexes with the Cas protein and targets a first target site, and a second DNA-targeting module comprising (a) the fusion protein of the first DNA-targeting module, and (b) a second gRNA that complexes with the Cas protein and targets a second target site. It will be understood that providing two or more different gRNAs for a given Cas protein allows the same Cas protein to be targeted to the target sites of the two or more gRNAs. Conversely, different Cas protein variants (e.g. SpCas9 and SaCas9) are compatible with different gRNA scaffold sequences and PAMs. Thus, it is possible to engineer a single DNA-targeting system comprising multiple non-overlapping CRISPR/Cas-based DNA-targeting modules.

The provided embodiments relate to compositions and methods for promoting T cell function, such as one or more T cell effector functions, by epigenetically modifying target sites in one or more target genes. In some embodiments, the methods can be used in connection with T cell therapies, such as in connection with adoptive T cell therapies. In some embodiments, modulating transcription of the one or more genes increases or improves one or more T cell phenotype or function. In some embodiments, a T cell effector function is increased, such as the ability to produce cytokines, for example IL-2 or IFN-gamma (IFNg), the ability of T cells to proliferate, the ability of T cells to kill target cells, or the ability of T cells to exhibit a persistent immune response. In particular embodiments, modulation of the one or more genes improves T cell effector functions after or upon T cell stimulation, including following serial stimulation that mimic conditions of repeated antigen encounter as occurs in vivo.

The administration of T cells targeting a specific antigen, also known as Adoptive Cell Therapy (ACT), is a promising approach for treating diseases such as cancer. However, current ACT treatments face challenges including suboptimal T cell function, expansion, and persistence. Furthermore, the persistence and functionality of the transferred T cells can significantly differ between different T cell subsets and among T cells from different patients. Recent clinical trials for ACT suggest that the ability to persist long term in the circulation is dependent on the differentiation stage of the T cell, including the ability to retain a network of transcription factors and metabolic regulators (Pilipow K., et. al., Journal of Clinical Investigation Insight 2018; 3(18):e122299). The T cells transferred into the patient are often terminally differentiated and therefore fail to persist in the long term, ultimately limiting effective anti-tumor response. For instance, while the first CAR-T cell therapy was FDA-approved as a cell & gene therapy in 2017, patients whose cancer relapse or do not respond to treatment often suffer from lack of CAR T cell persistence (Mueller et al, Blood (2018)). Moreover, no durable benefit has yet been observed for CAR T cell therapies in solid tumors.

Strategies to mitigate these challenges and enhance the persistence, expansion, and anti-tumor activity of chimeric antigen receptor (CAR) engineered T cells have been tested in preclinical and clinical settings. For instance, strategies for optimizing ex vivo T cell culture conditions, including the addition of cytokines during manufacturing (Besser M. J., Cytotherapy 2009; 11(2):206-17), expression of cytokines and/receptors by the CAR T cells (Krenciute G., Cancer Immunol Res. 2017 07; 5(7):571-581), use of pharmacological inhibitors during expansion to inhibit signaling pathways such as AKT (Urak R. et. al., Journal of Immunotherapy Cancer 2017 Mar. 21; 5:26) or PI3K (Peterson C. T et. al., Blood Advances 2018 Feb. 13; 2(3):210-223), immune-depletion and checkpoint blockade (Cherkassky L. et. al., Journal of clinical investigation 2016 Aug. 1; 126(8):3130-44) have been so far explored. However, existing strategies have not been entirely satisfactory. In some cases, concerns regarding cytokine-induced toxicity or the emergence of lymphoproliferative diseases as a result of the above-mentioned strategies have raised questions for alternative approaches.

The provided embodiments relate to identification of genomic locations that are epigenetically modified in a T cell to impact or promote T cell effector functions upon T cell stimulation, including those induced in a TCR and/or CAR-induced or dependent manner, such as demonstrated by assessment for cells producing IL-2 and/or IFNg, having the ability to proliferate, or having the ability to kill target cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. In some embodiments, the one or more agents are PMA and ionomycin. In some embodiments, the T cell stimulation is an antigen-specific stimulation, in which the cells ate stimulated with an agent providing an antigen or epitope thereof that is specific to, or recognized by, an antigen receptor (e.g. CAR) expressed on the T cell. For instance, the stimulating agent may include antigen-expressing target cells. In particular embodiments, the phenotype is or includes the production or secretion of a cytokine, such as IL-2 or IFN-g, in response to a T cell stimulation. The production and/or the secretion of cytokines contributes to immune responses, and is involved in different processes including the induction of anti-viral proteins and the induction of T cell proliferation. Cytokines are not pre-formed factors but are rapidly produced and secreted in response to cellular activation. The production or secretion of cytokines may be measured, detected, and/or quantified by any suitable technique known in the art.

In certain embodiments, the T cell function is the production of one or more cytokines. In particular embodiments, the production of one or more cytokines is measured, detected, and/or quantified by intracellular cytokine staining. Intracellular cytokine staining (ICS) by flow cytometry is a technique well-suited for studying cytokine production at the single-cell level. It detects the production and accumulation of cytokines within the cell (such as within the endoplasmic reticulum) after cell stimulation, allowing for the identification of cell populations that are positive or negative for production of a particular cytokine or for the separation of high producing and low producing cells based on a threshold. ICS can also be used in combination with other flow cytometry protocols for immunephenotyping using cell surface markers or with MHC multimers to access cytokine production in a particular subgroup of cells, making it a flexible and versatile method. Other single-cell techniques for measuring or detecting cytokine production include, but are not limited to ELISPOT, limiting dilution, and T cell cloning.

Notably, the target genes, and target sites therein, of the present disclosure were identified by a screening method involving transient delivery, in which the DNA-binding domain-effector fusion proteins (also called “epi-editors”) were delivered to the T cell transiently (i.e. delivered by a method that results in transient expression and/or presence of the fusion protein in the T cell) followed by primary or serial stimulation of the T cells to assess impact on functional T cell cytokines. It was found herein that the transient delivery of the epigenetic modifying DNA-targeting systems allowed identification of genomic targets whose modulation substantially impacts T cell function, but without requiring permanent presence of the epigenetic modifying DNA-targeting systems, and/or stable knock down or knockout of the target gene. This approach is advantageous because it permits identification of target genes and target sites that provide a better safety profile as their modulation is not reliant on a permanent editor integration, such as by lentiviral transduction. Moreover, the transient screening strategies allow for identification of target genes and target sites therein in which there is a durability of the effect of the epigenetic modifying DNA-targeting system that is not masked as a result of permanent integration into the genome and expression therefrom. This is in contrast to other screening approaches in which lentiviral delivery of DNA-systems has been employed (Schmidt et al. 2022 Science, 375, DOI: 10.1126/science.abj4008; Freimer et al. 2022 Nature Genetics, 54:1133-1144).

The provided embodiments can be used to target genes that when transcriptionally altered by epigenetic modification, can vastly facilitate or promote T cell function, including effector activities required for T cell persistence and function. Such a T cell profile is expected to produce durable effector functions, have better fitness/proliferation benefit, and have the ability to produce pro-proliferation cytokines (e.g. IL-2) and/or cytotoxic cytokines (e.g. IFNg) upon TCR or antigen stimulation. In particular, the provided embodiments provide for epigenetic-modifying DNA-targeting systems (i.e. “epi-editing systems”) and methods that can provide for long-lasting effector function with better fitness. This approach offers substantial clinical solutions to circumvent the problems with T cell persistence, suboptimal functionality, and/or exhaustion. Moreover, the epigenetic modification of the cell does not modify DNA at the sequence level, thereby avoiding safety concerns with gene editing approaches. The ability to epigenetically control the differentiation fate of T cells provides an advantageous approach for increasing the percentage or number of T cells in a population of T cells.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DNA-TARGETING SYSTEMS

In some embodiments, provided are DNA-targeting systems capable of specifically targeting a target site for at least one gene (i.e. a target gene), and modulating transcription of the at least one gene. In some embodiments, the at least one gene is one or more gene in a lymphoid cell, such as a T cell. In some embodiments, the target site for a gene is a target site in the gene or a regulatory DNA element thereof. In some embodiments, the transcription modulation is decreased transcription of each target gene. In some embodiments, the transcription modulation is increased transcription of each target gene. In provided embodiments, for each target gene that is targeted, the DNA-targeting system includes a fusion protein that comprises a DNA-binding domain that binds to the target site for the gene, and an effector domain for modulating transcription of the gene. In some embodiments, the provided DNA-targeting systems are able to modulate, such as repress or increase, transcription of the at least one gene in the cell. In some embodiments, transcriptional modulation of gene expression by the DNA-targeting systems provided herein can promote or improve function of the lymphoid cells. In particular embodiments, the provided DNA-targeting systems promote T cell function, such as one or more T cell effector functions, by epigenetically modifying target sites in the one or more target genes.

In some embodiments, the at least one effector domain is a transcriptional repressor effector domain for repressing transcription of each of the at least one gene (e.g. inhibits or reduces transcription of the gene as compared to transcription of the gene in the absence of the DNA-targeting system), such as any effector domain for transcriptionl repression described in Section I.E.1. In some embodiments, the effector domain is a transcriptional repressor effector domain, and the one or more genes are selected from the group consisting of: CBLB, CCNC, CD5, CISH, DGKZ, ELOB, FAS, Fli1, GATA3, KDM1A, MED12, MYB, PRDM1, TGFBR2, and RASA2. In some embodiments, the effector domain is a transcriptional repressor effector domain, and the one or more genes are selected from the group consisting of: CBLB, CISH, MED12, MYB, PRDM1, and RASA2.

In some embodiments, the effector domain directly or indirectly leads to reduced transcription of the gene. In some embodiments, the effector domain induces, catalyzes or leads to transcription repression. In some embodiments, the effector domain induces transcription repression. In some aspects, the effector domain is selected from a KRAB domain, ERF repressor domain, MXI1 domain, SID4X domain, MAD-SID domain, a DNMT family protein domain (e.g. DNMT3A or DNMT3B), a fusion of one or more DNMT family proteins or domains thereof (e.g. DNMT3A/L, which comprises a fusion of DNMT3A and DNMT3L domains), LSD1, EZH2, a partially or fully functional fragment or domain of any of the foregoing, or a combination of any of the foregoing. In some embodiments, the effector domain is KRAB. In some embodiments, the effector domain is DNMT3A/L.

In some embodiments, the at least one effector domain is a transcriptional activator effector domain for increasing transcription of each the at least one gene (e.g. activates or increases transcription of the gene as compared to transcription of the gene in the absence of the DNA-targeting system), such as any effector domain for transcriptional activation described in Section I.E.2. In some embodiments, the effector domain is a transcriptional activator effector domain, and the one or more genes are selected from the group consisting of: BATF, CD28, EOMES, IL-2, IL2RB, IRF4, LAT, LCP2, TBX21, and VAV1. In some embodiments, the effector domain is a transcriptional activator effector domain, and the one or more genes are selected from the group consisting of: EOMES, IL-2, LCP2, and TBX21.

In some embodiments, the effector domain directly or indirectly leads to increased transcription of the gene. In some embodiments, the effector domain induces, catalyzes or leads to transcription activation. In some embodiments, the effector domain induces transcription activation. In some aspects, the effector domain comprises: a VP64 domain, a p65 activation domain, a p300 domain, an Rta domain, a CBP domain, a VPR domain, a VPH domain, an HSF1 domain, a TET protein domain, optionally wherein the TET protein is TET1, a SunTag domain, or a domain, portion, variant, or truncation of any of the foregoing. In some embodiments, the effector domain is VP64.

In some embodiments, the DNA-targeting system includes a fusion protein comprising (a) at least one DNA-binding domain capable of being targeted to the target site; and (b) at least one effector domain capable of modulating transcription of the gene. In some embodiments, the at least one effector domain is a transcription repressor effector domain. In some embodiments, the at least one effector domain is a transcription activator effector domain. The fusion protein can be any suitable fusion protein, for example as described in Section I.F.

In some embodiments, the DNA-binding domain comprises or is derived from a CRISPR associated (Cas) protein, a zinc finger protein (ZFP), a transcription activator-like effector (TALE), meganuclease, homing endonuclease, I-SceI enzyme, or variants thereof. In some embodiments, the DNA-binding domain comprises a catalytically inactive (e.g. nuclease-inactive or nuclease-inactivated) variant of any of the foregoing. In some embodiments, the DNA-binding domain comprises a deactivated Cas9 (dCas9) protein or variant thereof that is a catalytically inactivated so that it is inactive for nuclease activity and is not able to cleave the DNA. The DNA-binding domain can be any suitable DNA-binding domain, for example as described in Sections I.C and I.D.

In some embodiments, the DNA-binding domain comprises or is derived from a Cas protein or variant thereof, such as a nuclease-inactive Cas or dCas (e.g. dCas9, and the DNA-targeting system comprises one or more guide RNAs (gRNAs), such as a combination of gRNAs (e.g. two gRNAs or three gRNAs). In some embodiments, the gRNA comprises a spacer sequence that is capable of targeting and/or hybridizing to the target site. In some embodiments, the gRNA is capable of complexing with the Cas protein or variant thereof. In some aspects, the gRNA directs or recruits the Cas protein or variant thereof to the target site. The gRNA can be any suitable gRNA, for example as described in section I.C.2.

In some embodiments, the DNA-targeting system is for repressing transcription of at least one gene, such as any described in Section I.B.2, and the fusion proteinof a DNA-targeting module thereof is a dCas9-KRAB fusion protein. In some embodiments, the fusion protein is a dCas9-KRAB-DNMT3A/L fusion protein. In some embodiments, the fusion protein is any as described herein, for example in Section I.F.

In some embodiments, the DNA-targeting system is for increasing transcription of at least one gene, such as any described in Section I.B.3, and the fusion protein of a DNA-targeting module thereof is a dCas9-VP64 fusion protein, such as a dCas9-2xVP64 fusion protein. In some embodiments, the fusion protein is any as described herein, for example in Section I.F.

Exemplary components and features of the DNA-targeting systems are provided below in the following subsections.

### A. DNA-Targeting Modules and Multiplexed DNA-Targeting Systems

In some embodiments, the DNA-targeting system contains at least one DNA-targeting module, where each DNA-targeting module of the system is a component of the DNA-targeting system that is independently capable of targeting one target site for a target gene. In some embodiments, each DNA-targeting module includes (a) a DNA-binding domain capable of being targeted to the target site, and (b) an effector domain for modulating transcription of the gene. In some embodiments, the DNA-targeting system comprises a single DNA-targeting module for targeted transcriptional modulation of a single gene.

In some embodiments, a DNA-targeting module is a CRISPR/Cas-based DNA-targeting module. In some embodiments, in a CRISPR/Cas-based DNA-targeting module, the DNA-binding domain of the fusion protein is a Cas protein or variant thereof (e.g. a dCas protein, such as dCas9) and the DNA-targeting module further comprises a gRNA for targeting the DNA-binding domain to the target site.

In some embodiments, a DNA-targeting module is a zinc finger protein (ZFP)-based DNA-targeting module. In some embodiments, in a ZFP-based DNA-targeting module, the DNA-binding domain of the fusion protein is an engineered zinc finger protein (eZFP).

In some embodiments, a DNA-targeting module is a transcription activator-like effector (TALE)-based DNA-targeting module. In some embodiments, in a TALE-based DNA-targeting module, the DNA-binding domain of the fusion protein is an engineered TALE.

In some embodiments, the DNA-targeting system includes a plurality of DNA-targeting modules, in which each DNA-targeting module targets a different target site. In some embodiments, one or more target sites are for different genes. In some embodiments, one or more target sites are for the same gene. In some embodiments, the DNA-targeting system is a multiplexed DNA-targeting system, i.e. is targeted to target sites for more than one gene. Hence, the term DNA-targeting system may include a multiplexed epigenetic-modifying DNA targeting system that includes more than one DNA-targeting module. In some embodiments, a multiplexed epigenetic-modifying DNA targeting system comprises at least 2, at least 3, at least 4, at least 5, at least 6, at least 7, at least 8, at least 9, at least 10, at least 20, at least 30, or more DNA-targeting modules. In some embodiments, a multiplexed epigenetic-modifying DNA-targeting system comprises 2 DNA-targeting modules. In some embodiments, a multiplexed epigenetic-modifying DNA-targeting system comprises 3 DNA-targeting modules.

In some embodiments, any two DNA-targeting modules of a DNA-targeting system can comprise separate (i.e. non-overlapping) components. For example, a DNA-targeting system may comprise a first DNA-targeting module comprising a first fusion protein with a DNA-binding domain (e.g. a ZFN or TALE-based DNA-binding domain) that targets a first target site, and a second DNA-targeting module comprising a second fusion protein with a second DNA-binding domain (e.g. a ZFN or TALE-based DNA-binding domain) that targets a second target site.

In some embodiments, any two DNA-targeting modules of a DNA-targeting system can comprise shared (i.e. overlapping) components. For example, a DNA-targeting system may comprise: i) a first DNA-targeting module comprising (a) a fusion protein comprising a Cas protein and an effector domain, and (b) a first gRNA that complexes with the Cas protein and targets a first target site, and ii) a second DNA-targeting module comprising (a) the fusion protein of the first DNA-targeting module, and (b) a second gRNA that complexes with the Cas protein and targets a second target site. It will be understood that providing two or more different gRNAs for a given Cas protein allows the Cas protein to be targeted to the target sites of the two or more gRNAs. Conversely, different Cas protein variants (e.g. SpCas9 and SaCas9) are compatible with different gRNA scaffold sequences and PAMs, as described herein. Thus, it is possible to engineer a single DNA-targeting system comprising multiple non-overlapping CRISPR/Cas-based DNA-targeting modules.

In some aspects, provided herein is an epigenetic-modifying DNA-targeting system comprising a plurality of DNA-targeting modules for modulating transcription of one or more genes. In some embodiments, the plurality of DNA-targeting modules comprises a first DNA-targeting module for modulating transcription of a first gene of the one or more genes, and a second DNA-targeting module for modulating transcription of a second gene of the one or more genes. In some embodiments, each DNA-targeting module comprises a fusion protein comprising: (a) a DNA-binding domain for targeting a target site of the target gene for the DNA-targeting module, and (b) at least one effector domain. In some embodiments, each DNA-targeting module comprises a transcriptional repressor effector domain for repressing transcription of the at least one gene. In some embodiments, each DNA-targeting module comprises a transcriptional activator effector domain for increasing transcription of the at least one gene.

B. Target Genes and Target Sites for Promoting Lymphocyte (e.g. T Cell) Activation and Function

In some aspects, provided herein are target sites in one or more target genes in which modulation of the target gene promotes T cell activation or function. In some embodiments, the target site is targeted using any of the provided DNA-targeting systems.

In some embodiments, the target site is in a gene in which reduced expression of the gene promotes T cell activation or function, such as any one or more of the target genes described in Section I.B.2. In some embodiments, the target site is a target site in a gene selected from the group consisting of CBLB, CCNC, CD5, CISH, DGKZ, ELOB, FAS, Fli1, GATA3, KDM1A, MED12, MYB, PRDM1, TGFBR2, and RASA2. In some embodiments, the target site is a target site in the CBLB gene. In some embodiments, the target site is a target site in the CISH gene. In some embodiments, the target site is a target site in the MED12 gene. In some embodiments, the target site is a target site in the MYB gene. In some embodiments, the target site is a target site in the PRDM1 gene. In some embodiments, the target site is a target site in the RASA2 gene.

In some embodiments, the target site is in a gene in which increased expression of the gene promotes T cell activation or function, such as any one or more of the target genes described in Section I.B.3. In some embodiments, the target site is a target site in a gene selected from the group consisting of BATF, CD28, EOMES, IL-2, IL2RB, IRF4, LAT, LCP2, TBX21, and VAV1. In some embodiments, the target site is a target site in the EOMES gene. In some embodiments, the target site is a target site in the IL-2 gene. In some embodiments, the target site is a target site in the LCP2 gene. In some embodiments, the target site is a target site in the TBX21 gene.

In some embodiments, the target site is targeted by a DNA-targeting system, such as by a DNA-targeting module of the DNA-targeting system, such as any described herein. In some embodiments, the target site is a target site for a gene (e.g., a target gene). In some embodiments, the target site for a gene is in the gene or a regulatory DNA element thereof. In some embodiments, the target site is a target site in the gene. In some aspects, the gene is a target gene. In some embodiments, the target gene is a gene in a cell. In some embodiments, the cell is an immune cell, such as a T cell. In some embodiments, provided herein are multiplexed epigenetic-modifying DNA-targeting systems that target a combination of at least two target genes or regulatory DNA elements thereof described herein.

In some embodiments, the DNA-targeting system targets to or binds to a target site in a gene, such as any described herein. In some embodiments, the target site is located in the gene and/or a regulatory DNA element of the gene. In some embodiments, a regulatory DNA element is a sequence to which a gene regulatory protein may bind and affect transcription of the gene. In some embodiments, the regulatory DNA element is a cis, trans, distal, proximal, upstream, or downstream regulatory DNA element of a gene. In some embodiments, the regulatory DNA element is a promoter or enhancer of the gene. In some embodiments, the target site is located within a promoter, enhancer, exon, intron, untranslated region (UTR), 5′ UTR, or 3′ UTR of the gene. In some embodiments, the regulatory DNA element is a promoter. In some embodiments, a promoter is a nucleotide sequence to which RNA polymerase binds to begin transcription of the gene. In some embodiments, a promoter is a nucleotide sequence located within about 100 bp, about 500 bp, about 1000 bp, or more, of a transcriptional start site of the gene. In some embodiments, a promoter is within 500 bp of a transcriptional start site of the gene. In some embodiments the target site is located within a sequence of unknown or known function that is suspected of being able to control expression of a gene.

### 1. Lymphoid Cells and Modulated Effector Functions

In some embodiments, the provided DNA-targeting systems and/or a DNA-targeting provide for transcriptional modulation to repress or increase expression of at least one target gene. In some embodiments, the target gene is a gene for which expression of the gene regulates a cellular phenotype. In some embodiments, the target gene is capable of regulating a phenotype in a T cell. In some embodiments, modulated expression of the gene, such as increased transcription or decreased transcription, regulates the phenotype. In some embodiments, modulated expression of the gene promotes increased T cell effector function upon T cell stimulation. In some embodiments, the increased T cell effector function is increased compared to a T cell in which expression of the gene has not been modulated with a provided DNA-targeting system. Methods for modulating T cell function or functions of other lymphoid cells by provided DNA-targeting systems are further described below and in Section IV.

In some embodiments, the gene is modulated by a DNA-targeting system, such as any DNA-targeting system provided herein. In some embodiments, the DNA-targeting system is transiently delivered to the cell. In some embodiments, delivery of the DNA-targeting system, for example by transient delivery, promotes increased T cell effector function upon T cell stimulation. In some embodiments, the T cell effector function is increased in comparison to a comparable T cell to which the DNA-targeting system has not been delivered.

In some aspects, transient delivery refers to any method of delivery that results in expression and/or presence of one or more components of the DNA-targeting system in the cell for a limited duration. For example, delivery of mRNA (such as by electroporation) encoding the fusion protein of the DNA-targeting system to a cell can result in transient expression of the fusion protein in the cell, for example until the mRNA is degraded. In other examples, the DNA-targeting system can be expressed from one or more nucleic acids encoding the DNA-targeting system, wherein the nucleic acids encoding the DNA-targeting system are not incorporated into the genome of the cell, and are eventually degraded and/or removed from the cell such that expression of the DNA-targeting system does not persist. In other examples, one or more components of the DNA-targeting system, such as a fusion protein and optionally a gRNA can be synthesized in vitro and delivered directly to the cell (e.g. by electroporation) without the need for an expression vector, resulting in transient presence of the DNA-targeting system, for example until the fusion protein and/or gRNA are degraded. In some aspects, transient delivery differs from non-transient methods of delivery that result in stable expression, such as methods involving incorporation of an expression vector for a DNA-targeting system or component thereof into the genome of the cell.

In some embodiments, delivery of the DNA-targeting system to the cell (e.g. T cell), such as by transient delivery, promotes a phenotype in the cell (e.g. T cell). In some embodiments, the phenotype is increased activation or function in the cell (e.g. T cell). In some embodiments, delivery of the DNA-targeting system to the cell (e.g. T cell), such as by transient delivery, promotes increased activation or function in the cell (e.g. T cell). In some embodiments, the phenotype is increased T cell effector function upon T cell stimulation. In some embodiments, the T cell effector function is increased compared to a T cell that has not been delivered the epigenetic-modifying DNA-targeting system. In some embodiments, decreased expression (e.g. transcription) of the one or more target genes, such as a target gene described in Section I.B.2, leads to increased T cell effector function upon T cell stimulation. In some embodiments, increased expression (e.g. transcription) of the one or more target genes, such as a target gene described in Section I.B.3, leads to increased T cell effector function upon T cell stimulation. In some embodiments, the T cell effector function is characterized by an activity selected from the group consisting of IL-2 production, IFN-gamma production, TNF-alpha production, T cell proliferation or a combination of any of the foregoing.

In some embodiments, provided DNA-targeting systems promote or increase an improved T cell effector function as may occur after stimulation in vitro, ex vivo or in vivo. In some embodiments, the T cell stimulation is a polyclonal T cell stimulation. In some embodiments, the T cell stimulation is with an anti-CD3 and anti-CD28 activation reagent. In some embodiments, the T cell stimulation is an antigen-specific activity that is mediated or induced by specific binding of an antigen to an antigen receptor on the surface of the T cell. In some embodiments, the T cell expresses a chimeric antigen receptor (CAR) or engineered T cell receptor (eTCR) directed against an antigen and the T cell stimulation is an antigen-specific stimulation of the CAR or eTCR. In some embodiments, the T cell stimulation is with antigen-expressing target cells. In some embodiments, the T cell stimulation occurs when the T cell contacts a cell expressing the antigen. In some embodiments, the T cell stimulation is a restimulation after at least one prior T cell stimulation of the T cells. In some embodiments, the T cells are stimulated and then are transiently delivered a provided DNA-targeting system prior to assessment of a T cell effector function or phenotype.

In certain embodiments, the cell composition that contains T cells is stimulated with an anti-CD3/anti-CD28 activation reagent for an amount of time, and an effector function is measured at one or more time points during or after the incubation. In some embodiments, such an activation reagent has anti-CD3/anti-CD28 coated on a support, such as magnetic beads or other matrix. Exemplary activation reagent Dynabeads™ or T cell TransAct™. In some embodiments, the T cells are incubated with the activation reagent, for 3 hours to 72 hours, such as 12 hours to 48 hours, for example, 12 hours, 18 hours, 24 hours, 36 hours, or 48 hours, or any value between any of the foregoing. In some embodiments, cells can be assessed directly for an effector function, such as production of cytokines or ability to proliferate. In some embodiments, the supernatant of the culture can be collected and the amount of a soluble factor, e.g., a cytokine is detected. In some embodiments, the T cells can be collected and re-exposed to the activation reagent to monitor cytolytic activity. In some embodiments, cells can be restimulated one or more time, such as by serial stimulation methods, and serially assessed for effector functions after each stimulation.

In certain embodiments, the antigen-specific activity is measured by incubating the cell composition that contains T cells expressing the antigen receptor, e.g., a CAR, with antigen-expressing cells for an amount of time, and an effector function is measured at one or more time points during or after the incubation. In some embodiments, the T cells are incubated with the antigen specific agent, such as antigen-expressing cells, for 3 hours to 96 hours, such as 12 hours to 72 hours, for example, 12 hours, 24 hours, 48 hours, 72 hours or any value between any of the foregoing. In some embodiments, cells can be assessed directly for an effector function, such as production of cytokines or ability to proliferate. In some embodiments, the supernatant of the culture can be collected and the amount of a soluble factor, e.g., a cytokine is detected. In some embodiments, the T cells can be collected and re-exposed to antigen-expressing target cells to monitor cell killing (cytolytic activity) of target cells. In some embodiments, cells can be restimulated one or more time, such as by serial stimulation methods, and serially assessed for effector functions after each stimulation. In some embodiments, the T cells with the engineered antigen receptor (e.g., a CAR) are incubated with a constant number of the antigen-expressing cells, such as at an effector to target (E:T) ratio of 1:4 to 4:1, such as at a ratio of 1:4, 1:3, 1:2 or 1:1.

In some embodiments, the cell (e.g. T cell) exhibits increased cytokine production. In some embodiments, the increased cytokine production occurs upon T cell stimulation. In some embodiments, T cell effector function is characterized by cytokine production. In some embodiments, the cytokine production is increased by at least about 1.1 fold, 1.2 fold, 1.3 fold, 1.4 fold, 1.5 fold, 2 fold, 5 fold, 10 fold, 50 fold, 100 fold, or greater, in comparison to a cell that has not been delivered the epigenetic-modifying DNA-targeting system. In some embodiments, the cytokine production Is production of IL-2, IFN-gamma, TNF-alpha, or a combination thereof. In some embodiments, the T cell effector function is characterized by IL-2 production. In some embodiments, the cell (e.g. T cell) exhibits increased IL-2 production. In some embodiments, the T cell effector function is characterized by IFN-gamma production. In some embodiments, the cell (e.g. T cell) exhibits increased IFN-gamma production. In some embodiments, the T cell effector function is characterized by IL-2 production and IFN-gamma production. In some embodiments, the cell (e.g. T cell) exhibits increased IL-2 production and increased IFN-gamma production. In some embodiments, the T cell effector function is characterized by polyfunctional production of IL-2, IFN-gamma and TNF-alpha. In some embodiments, the cell (e.g. T cell) exhibits increased IL-2, IFN-gamma and TNF-alpha production.

Suitable techniques for the measurement of the production or secretion of a soluble factor, such as a cytokine, are known in the art. Production and/or secretion of a soluble factor can be measured by determining the concentration or amount of the extracellular amount of the factor, or determining the amount of transcriptional activity of the gene that encodes the factor. Suitable techniques include, but are not limited to assays such as an immunoassay, an aptamer-based assay, a histological or cytological assay, an mRNA expression level assay, an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay, protein microarrays, high-performance liquid chromatography (HPLC), Meso Scale Discovery (MSD) electrochemiluminescence and bead based multiplex immunoassays (MIA). In some embodiments, the suitable technique may employ a detectable binding reagent that specifically binds the soluble factor.

In some embodiments, the cytokine production is measured as a percentage of cells being positive for the cytokine, for example as measured by intracellular cytokine staining (ICS) and flow cytometry. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning.

In some embodiments, the cytokine production is measured as the amount of cytokine secreted from the cell, for example as measured by ELISA (enzyme-linked immunosorbent assay). ELISA is a plate-based assay technique designed for detecting and quantifying substances such as peptides, cytokines, antibodies and hormones. In an ELISA, the soluble factor, such as a cytokine, must be immobilized to a solid surface and then complexed with an antibody that is linked to an enzyme. Detection is accomplished by assessing the conjugated enzyme activity via incubation with a substrate to produce a detectable signal.

In some embodiments, the T cell effector function is characterized by activity that further comprises T cell proliferation. In some embodiments, the cell (e.g. T cell) exhibits increased proliferation. In some embodiments, the increased proliferation occurs upon T cell stimulation. In some embodiments, the proliferation is increased by at least about 1.1 fold, 1.2 fold, 1.3 fold, 1.4 fold, 1.5 fold, 2 fold, 5 fold, 10 fold, 50 fold, 100 fold, or greater, in comparison to a cell that has not been delivered the epigenetic-modifying DNA-targeting system. In some embodiments, the proliferation is measured as the increase in cell numbers before and after stimulation. In some embodiments, the increased proliferation is measured as the number of cells after stimulation in a cell population delivered with the epigenetic-modifying DNA-targeting system compared to the number of cells after stimulation in a cell population not delivered with the epigenetic-modifying DNA-targeting system. In some embodiments, the cell (e.g. T cell) does not exhibit increased proliferation.

In some embodiments, the T cell effector function is characterized by activity that further comprises killing of target cells. In some embodiments, the cell (e.g. T cell) exhibits increased killing of target cells. In some embodiments, the increased killing of target cells occurs upon T cell stimulation. In some embodiments, the stimulation is performed by contacting the cells (e.g. T cells) with target cells. In some embodiments, T cells are incubated with antigen-expressing target cells at ratios between 4:1 and 1:4, inclusive, such as at ratios of 1:4, 1:3, 1:2 or 1:1. In some embodiments, the killing of target cells is increased by at least about 1.1 fold, 1.2 fold, 1.3 fold, 1.4 fold, 1.5 fold, 2 fold, 5 fold, 10 fold, 50 fold, 100 fold, or greater, in comparison to a cell that has not been delivered the epigenetic-modifying DNA-targeting system. In some embodiments, the killing is measured as the ability of the cells to kill target cells when contacted by the target cells. Killing of target cells can be measured by any suitable assay, for example as described herein in the Examples. In some embodiments, the killing is measured in an in vitro assay, wherein cells delivered with the epigenetic-modifying DNA-targeting system are co-cultured with the target cells, and the number of target cells are measured over time. In some embodiments, reduced numbers and/or proliferation of the target cells are indicative of target cell killing. The cytolytic activity can be measured by directly or indirectly measuring the target cell number over time. For example, the target cells may be incubated with a detectable marker prior to being incubated with antigen receptor (e.g. CAR)-expressing cells, such a marker that is detectable then the target cell is lysed, or a detectable marker that is detectable in viable target cells. These readouts provide direct or indirect of target cell number and/or target cell death, and can be measured at different time points during the assay. A reduction of target cell number and/or an increase of target cell death indicate the cytolytic activity of the cells. Suitable methods for performing cytolytic assays are known in the art, and include, but are not limited to chromium-51 release assays, non-radioactive chromium assays, flow cytometric assays that use fluorescent dyes such as carboxyfluorescein succinimidyl ester (CFSE), PKH-2, and PKH-26.

In some embodiments, the T cell effector function is characterized by activity that further comprises T cell persistence. In some embodiments, the cell (e.g. T cell) exhibits increased persistence (e.g. T cell persistence). In some embodiments, persistence relates to the ability of cells to remain present and/or maintain an immune response in the presence of target cells. In some embodiments, persistence can be measured in vitro or in vivo, for example after administration of cells to a subject. Persistence can be measured by any suitable method, for example as described in Section IV.

In certain embodiments, the ability of T cells to persist can be measured as a pharmacokinetic property of the cell composition following its administration to a subject. In some embodiments, the pharmacokinetic parameter can include the exposure, number, concentration, persistence and proliferation. In some cases, pharmacokinetics can be assessed by measuring such parameters as the maximum (peak) plasma concentration (Cₘₐₓ), the peak time (i.e. when maximum plasma concentration (Cₘₐₓ) occurs; Tₘₐₓ), the minimum plasma concentration (i.e. the minimum plasma concentration between doses of a therapeutic agent, e.g., CAR+ T cells; Cₘᵢₙ), the elimination half-life (T_{1/2}) and area under the curve (i.e. the area under the curve generated by plotting time versus plasma concentration of the therapeutic agent CAR+ T cells; AUC), following administration. The parameters of the administered engineered T cells can be measured in samples of blood from the subject. For example, nucleic acid-based methods, such as quantitative PCR (qPCR) or flow cytometry-based methods, or other assays, such as an immunoassay, ELISA, or chromatography/mass spectrometry-based assays can be used.

In some aspects, nucleic acid-based methods, such as quantitative PCR (qPCR), is used to assess the quantity of cells expressing the antigen receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., CAR, per microgram of DNA, or as the number of antigen receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, the primers or probe used for qPCR or other nucleic acid-based methods are specific for binding, recognizing and/or amplifying nucleic acids encoding the antigen receptor, and/or other components or elements of the plasmid and/or vector, including regulatory elements, e.g., promoters, transcriptional and/or post-transcriptional regulatory elements or response elements, or markers, e.g., surrogate markers. In some embodiments, the primers can be specific for regulatory elements, such as the woodchuck hepatitis virus post-transcriptional regulatory element (WPRE).

In some embodiments, any of the phenotypes described herein, such as increased IL-2 production, increased IFN-gamma production, increased IL-2 production and increased IFN-gamma production, increased IL-2, IFN-gamma and TNF-alpha production, increased proliferation or proliferation that is not increased, increased killing of target cells, and/or increased persistence, are observed after stimulation (e.g. T cell stimulation).

In some embodiments, the phenotype, such as any phenotype described herein, including increased T cell effector function, occurs 48 hours or more after the transient delivery of the epigenetic-modifying DNA-targeting system to the T cell. In some embodiments, the phenotype, such as increased T cell effector function, occurs up to 6 days, up to 9 days, up to 12 days, up to 15 days, up to 21 days, up to 28 days, up to 35 days, up to 42 days, up to 49 days, up to 56 days, up to 63 days, up to 71 days or more after the transient delivery of the epigenetic-modifying DNA-targeting system to the T cell.

In some aspects, the phenotype is one that is characterized by a cell surface phenotype of the cells. In some embodiments, the phenotype comprises expression of one or more cell-surface markers selected from IL-2+, TNFa+, IFNg+, or any combination thereof. In some embodiments, the phenotype is a phenotype in a T cell, such as a CD3+ T cell, which may be a CD4+ T-cell or CD8+ T cell. Thus, in some embodiments, the phenotype comprises expression of one or more cell-surface markers selected from CD3+, CD4+, CD8+, IL-2+, TNFa+, IFNg+, or any combination thereof. In some aspects, the phenotype comprises expression of IL-2+. In some embodiments, the phenotype comprises expression of IL-2− and IFNg+.

It is understood that embodiments of the provided epigenetic-modifying DNA-targeting systems are not limited to modulating expression of target genes and promoting phenotypes in T cells, but may also be used to modulate any one or more of the target genes as described herein in any lymphoid cell. In addition to T cells, lymphoid cells can include NK cells, NKT cells, any cells that have been differentiated from stem cells into such lymphoid cells and/or have been differentiated from progenitor cells, such as common lymphoid progenitors (CLPs). In some embodiments, the lymphoid cells are differentiated from stem cells, such as hematopoietic stem or progenitor cells, or progenitor cells. In some embodiments, the lymphoid cells are trans-differentiated from a non-pluripotent cell of non-hematopoietic lineage.

In some embodiments, the lymphoid cell for modulation is an isolated or enriched population of lymphoid immune cells, such as a population isolated or enriched in T, NK and/or NKT cells. In some embodiments, the cells for modulation are isolated or enriched T cells. In some embodiments, the cells for modulation are isolated or enriched NK cells. In some embodiments, the cells for modulation are isolated or enriched NK T cells. In some embodiments, isolated or enriched populations or subpopulations of immune cells comprising T, NK, and/or NKT cells for modulation can be obtained from a unit of blood using any number of techniques known to the skilled artisan, such as Ficoll™ separation. In one embodiment, T, NK or NKT cells from the circulating blood of an individual are obtained by apheresis and separated from other nucleated white blood cells, red blood cells and platelets, such as by Ficoll™ separation or affinity-based selection. In some embodiments, the cells are primary cells. In some embodiments, the primary cells are isolated or enriched from a peripheral blood sample from a subject, such as a human subject.

In some embodiments, the lymphoid cells for modulation are differentiated in vitro from a stem cell or progenitor cell. In some embodiments, the lymphoid cells, such as T, NK or NKT cells or lineages thereof, can be differentiated from a stem cell, a hematopoietic stem or progenitor cell (HSC), or a progenitor cell. The progenitor cell can be a CD34+ hemogenic endothelium cell, a multipotent progenitor cell, a T cell progenitor, an NK cell progenitor, or an NKT cell progenitor. In some embodiments, the progenitor cell is a lymphoid progenitor cells such as a common lymphoid progenitor cell, early thymic progenitor cells, pre-T cell progenitor cells, pre-NK progenitor cell, T progenitor cell, NK progenitor cell or NKT progenitor cell. The stem cell can be a pluripotent stem cell, such as induced pluripotent stem cells (iPSCs) and embryonic stem cells (ESCs). The iPSC is a non-naturally occurring reprogrammed pluripotent cell. Once the cells of a subject have been reprogrammed to a pluripotent state, the cells can then be programmed or differentiated to a desired cell type or subtypes, such as T, NK, or NKT cells.

In some embodiments, the iPSC is differentiated to a T, NK or NKT cells by a multi-stage differentiation platform wherein cells from various stages of development can be induced to assume a hematopoietic phenotype, ranging from mesodermal stem cells, to fully differentiated T, NK or NKT cells (See e.g. U.S. Pat. No. 10,626,372).

In some embodiments, the population or subpopulation of lymphoid cells is trans-differentiated in vitro from a non-pluripotent cell of non-hematopoietic fate to a hematopoietic lineage cell or from a non-pluripotent cell of a first hematopoietic cell type to a different hematopoietic cell type, which can be a T, NK, or NKT progenitor cell or a fully differentiated specific type of immune cell, such as T, NK, or NKT cell (See e.g. U.S. Pat. No. 9,376,664 and U.S. application Ser. No. 15/072,769, the disclosure of which is incorporated herein in their entirety). In some embodiments, the non-pluripotent cell of non-hematopoietic fate is a somatic cell, such as a skin fibroblast, an adipose tissue-derived cell and a human umbilical vein endothelial cell (HUVEC). Somatic cells useful for trans-differentiation may be immortalized somatic cells.

Various strategies are being pursued to induce pluripotency, or increase potency, in cells (Takahashi, K., and Yamanaka, S., Cell 126, 663-676 (2006); Takahashi et al., Cell 131, 861-872 (2007); Yu et al., Science 318, 1917-1920 (2007); Zhou et al., Cell Stem Cell 4, 381-384 (2009); Kim et al., Cell Stem Cell 4, 472-476 (2009); Yamanaka et al., 2009; Saha, K., Jaenisch, R., Cell Stem Cell 5, 584-595 (2009)), and improve the efficiency of reprogramming (Shi et al., Cell Stem Cell 2, 525-528 (2008a); Shi et al., Cell Stem Cell 3, 568-574 (2008b); Huangfu et al., Nat Biotechnol 26, 795-797 (2008a); Huangfu et al., Nat Biotechnol 26, 1269-1275 (2008b); Silva et al., Plos Bio 6, e253. Doi: 10.1371/journal. Pbio. 0060253 (2008); Lyssiotis et al., PNAS 106, 8912-8917 (2009); Ichida et al., Cell Stem Cell 5, 491-503 (2009); Maherali, N., Hochedlinger, K., Curr Biol 19, 1718-1723 (2009b); Esteban et al., Cell Stem Cell 6, 71-79 (2010); and Feng et al., Cell Stem Cell 4, 301-312 (2009)), the disclosures of which are hereby incorporated by reference in their entireties.

It is understood that a cell that is positive (+) for a particular cell surface marker is a cell that expresses the marker on its surface at a level that is detectable. Likewise, it is understood that a cell that is negative (−) for a particular cell surface marker is a cell that expresses the marker on its surface at a level that is not detectable. Antibodies and other binding entities can be used to detect expression levels of marker proteins to identify or detect a given cell surface marker. Suitable antibodies may include polyclonal, monoclonal, fragments (such as Fab fragments), single chain antibodies and other forms of specific binding molecules. Antibody reagents for cell surface markers above are readily known to a skilled artisan. A number of well-known methods for assessing expression level of surface markers or proteins may be used, such as detection by affinity-based methods, e.g., immunoaffinity-based methods, e.g., in the context of surface markers, such as by flow cytometry. In some embodiments, the label is a fluorophore and the method for detection or identification of cell surface markers on cells (e.g. T cells) is by flow cytometry. In some embodiments, different labels are used for each of the different markers by multicolor flow cytometry. In some embodiments, surface expression can be determined by flow cytometry, for example, by staining with an antibody that specifically binds to the marker and detecting the binding of the antibody to the marker.

In some embodiments, a cell (e.g. T cell) is positive (pos or +) for a particular marker if there is detectable presence on or in the cell of a particular marker, which can be an intracellular marker or a surface marker. In some embodiments, surface expression is positive if staining by flow cytometry is detectable at a level substantially above the staining detected carrying out the same procedures with an isotype-matched control under otherwise identical conditions and/or at a level substantially similar to, or in some cases higher than, a cell known to be positive for the marker and/or at a level higher than that for a cell known to be negative for the marker.

In some embodiments, a cell (e.g. T cell) is negative (neg or −) for a particular marker if there is an absence of detectable presence on or in the cell of a particular marker, which can be an intracellular marker or a surface marker. In some embodiments, surface expression is negative if staining is not detectable by flow cytometry at a level substantially above the staining detected carrying out the same procedures with an isotype-matched control under otherwise identical conditions and/or at a level substantially lower than a cell known to be positive for the marker and/or at a level substantially similar to a cell known to be negative for the marker.

In some aspects, the phenotype can be characterized by one or more functions of the cells. In some aspects, the phenotype is characterized by polyfunctional activity of the T cells to produce more than one T cell stimulatory cytokine, such as determined in a polyfunctional cytokine secretion assay following stimulation of the T cells with a stimulatory agent. In some embodiments, the T cell is polyfunctional for producing two or more cytokines. In some embodiments, a T cell is polyfunctional for producing two or more cytokines selected from among interferon-gamma (IFN-gamma), interleukin 2 (IL-2) and TNF-alpha. In some embodiments, a polyfunctional T cell produces IFN-gamma, IL-2, and TNF-alpha. In some embodiments, the stimulatory agent is a non-specific or non-antigen-dependent T cell stimulatory agent. In some embodiments, the non-specific or non-antigen dependent T cell stimulatory agent is a polyclonal stimulatory agent. In some embodiments, the non-specific or non-antigen dependent stimulatory agent comprises PMA/ionomycin, anti-CD3/anti-CD28, phytohemagglutinin (PHA) or concanavalin A (ConA). In some embodiments, the non-specific or non-antigen dependent T cell stimulatory agent contains PMA/ionomycin.

In particular embodiments, the production of one or more cytokines is measured, detected, and/or quantified by intracellular cytokine staining. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. In some embodiments, as described above, the stimulation can be performed using nonspecific stimulation, e.g., is not an antigen-specific stimulation. For example, PMA/ionomycin can be used for nonspecific cell stimulation. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning. In some embodiments, the assays to assay polyfunctional cytokine secretion of multiple cytokines, can include multiplexed assays or other assays to assess polyfunctionality (see, e.g., Xue et al., (2017) Journal for ImmunoTherapy of Cancer 5:85).

### 2. Genes and Target Sites for Decreasing Transcription

In some embodiments, delivery of the DNA-targeting system represses (e.g. decreases) transcription of one or more target genes selected from the group consisting of: CBLB, CCNC, CD5, CISH, DGKZ, ELOB, FAS, Fli1, GATA3, KDM1A, MED12, MYB, PRDM1, TGFBR2, and RASA2. In some embodiments, provided herein are target sites for one or more genes for which reduced transcription promotes a phenotype in a cell. In some embodiments, provided herein are target sites for one or more genes for which reduced transcription promotes increased T cell effector function. In some embodiments, the reduced transcription promotes increased T cell effector function upon T cell stimulation. In some embodiments, the one or more genes are selected from CBLB, CCNC, CD5, CISH, DGKZ, ELOB, FAS, Fli1, GATA3, KDM1A, MED12, MYB, PRDM1, TGFBR2, and RASA2. In some embodiments, the one or more genes are selected from CBLB, CISH, MED12, MYB, PRDM1, and RASA2.

In some embodiments, the DNA-targeting system comprises a plurality of DNA-targeting modules. In some embodiments, each DNA-targeting module targets a target site. In some embodiments, the plurality of DNA-targeting modules target at least a first gene and a second gene, wherein the first and second gene are independently selected from the group consisting of CBLB, CCNC, CD5, CISH, DGKZ, ELOB, FAS, Fli1, GATA3, KDM1A, MED12, MYB, PRDM1, TGFBR2, and RASA2. In some embodiments, the plurality of DNA-targeting modules target a first gene and a second gene, wherein the first and second gene are independently selected from the group consisting of CBLB, CCNC, CD5, CISH, DGKZ, ELOB, FAS, Fli1, GATA3, KDM1A, MED12, MYB, PRDM1, TGFBR2, and RASA2.

In some embodiments, the plurality of DNA-targeting modules target at least a first gene, a second gene, and a third gene, wherein the first, second and third gene are independently selected from the group consisting of CBLB, CCNC, CD5, CISH, DGKZ, ELOB, FAS, Fli1, GATA3, KDM1A, MED12, MYB, PRDM1, TGFBR2, and RASA2. In some embodiments, the plurality of DNA-targeting modules target a first gene, a second gene, and a third gene, wherein the first, second and third gene are independently selected from the group consisting ofCBLB, CCNC, CD5, CISH, DGKZ, ELOB, FAS, Fli1, GATA3, KDM1A, MED12, MYB, PRDM1, TGFBR2, and RASA2.

In some embodiments, the DNA-targeting system targets a combination of genes set forth in Table 1. In some embodiments, the plurality of DNA-targeting modules target a combination of genes set forth in Table 1. In some embodiments, transcription of each of the genes of the combination is repressed by the DNA-targeting system.

TABLE 1
Combinations of genes targeted by a multiplexed
epigenetic-modifying DNA-targeting system for
decreasing transcription of target genes

| first gene | second gene | third gene |
|---|---|---|
| CBLB | CCNC | — |
| CBLB | CD5 | — |
| CBLB | CISH | — |
| CBLB | DGKZ | — |
| CBLB | ELOB | — |
| CBLB | FAS | — |
| CBLB | Fli1 | — |
| CBLB | GATA3 | — |
| CBLB | KDM1A | — |
| CBLB | MED12 | — |
| CBLB | MYB | — |
| CBLB | PRDM1 | — |
| CBLB | RASA2 | — |
| CBLB | TGFBR2 | — |
| CCNC | CD5 | — |
| CCNC | CISH | — |
| CCNC | DGKZ | — |
| CCNC | ELOB | — |
| CCNC | FAS | — |
| CCNC | Fli1 | — |
| CCNC | GATA3 | — |
| CCNC | KDM1A | — |
| CCNC | MED12 | — |
| CCNC | MYB | — |
| CCNC | PRDM1 | — |
| CCNC | RASA2 | — |
| CCNC | TGFBR2 | — |
| CD5 | CISH | — |
| CD5 | DGKZ | — |
| CD5 | ELOB | — |
| CD5 | FAS | — |
| CD5 | Fli1 | — |
| CD5 | GATA3 | — |
| CD5 | KDM1A | — |
| CD5 | MED12 | — |
| CD5 | MYB | — |
| CD5 | PRDM1 | — |
| CD5 | RASA2 | — |
| CD5 | TGFBR2 | — |
| CISH | DGKZ | — |
| CISH | ELOB | — |
| CISH | FAS | — |
| CISH | Fli1 | — |
| CISH | GATA3 | — |
| CISH | KDM1A | — |
| CISH | MED12 | — |
| CISH | MYB | — |
| CISH | PRDM1 | — |
| CISH | RASA2 | — |
| CISH | TGFBR2 | — |
| DGKZ | ELOB | — |
| DGKZ | FAS | — |
| DGKZ | Fli1 | — |
| DGKZ | GATA3 | — |
| DGKZ | KDM1A | — |
| DGKZ | MED12 | — |
| DGKZ | MYB | — |
| DGKZ | PRDM1 | — |
| DGKZ | RASA2 | — |
| DGKZ | TGFBR2 | — |
| ELOB | FAS | — |
| ELOB | Fli1 | — |
| ELOB | GATA3 | — |
| ELOB | KDM1A | — |
| ELOB | MED12 | — |
| ELOB | MYB | — |
| ELOB | PRDM1 | — |
| ELOB | RASA2 | — |
| ELOB | TGFBR2 | — |
| FAS | Fli1 | — |
| FAS | GATA3 | — |
| FAS | KDM1A | — |
| FAS | MED12 | — |
| FAS | MYB | — |
| FAS | PRDM1 | — |
| FAS | RASA2 | — |
| FAS | TGFBR2 | — |
| Fli1 | GATA3 | — |
| Fli1 | KDM1A | — |
| Fli1 | MED12 | — |
| Fli1 | MYB | — |
| Fli1 | PRDM1 | — |
| Fli1 | RASA2 | — |
| Fli1 | TGFBR2 | — |
| GATA3 | KDM1A | — |
| GATA3 | MED12 | — |
| GATA3 | MYB | — |
| GATA3 | PRDM1 | — |
| GATA3 | RASA2 | — |
| GATA3 | TGFBR2 | — |
| KDM1A | MED12 | — |
| KDM1A | MYB | — |
| KDM1A | PRDM1 | — |
| KDM1A | RASA2 | — |
| KDM1A | TGFBR2 | — |
| MED12 | MYB | — |
| MED12 | PRDM1 | — |
| MED12 | RASA2 | — |
| MED12 | TGFBR2 | — |
| MYB | PRDM1 | — |
| MYB | RASA2 | — |
| MYB | TGFBR2 | — |
| PRDM1 | RASA2 | — |
| PRDM1 | TGFBR2 | — |
| RASA2 | TGFBR2 | — |
| CBLB | CCNC | CD5 |
| CBLB | CCNC | CISH |
| CBLB | CCNC | DGKZ |
| CBLB | CCNC | ELOB |
| CBLB | CCNC | FAS |
| CBLB | CCNC | Fli1 |
| CBLB | CCNC | GATA3 |
| CBLB | CCNC | KDM1A |
| CBLB | CCNC | MED12 |
| CBLB | CCNC | MYB |
| CBLB | CCNC | PRDM1 |
| CBLB | CCNC | RASA2 |
| CBLB | CCNC | TGFBR2 |
| CBLB | CD5 | CISH |
| CBLB | CD5 | DGKZ |
| CBLB | CD5 | ELOB |
| CBLB | CD5 | FAS |
| CBLB | CD5 | Fli1 |
| CBLB | CD5 | GATA3 |
| CBLB | CD5 | KDM1A |
| CBLB | CD5 | MED12 |
| CBLB | CD5 | MYB |
| CBLB | CD5 | PRDM1 |
| CBLB | CD5 | RASA2 |
| CBLB | CD5 | TGFBR2 |
| CBLB | CISH | DGKZ |
| CBLB | CISH | ELOB |
| CBLB | CISH | FAS |
| CBLB | CISH | Fli1 |
| CBLB | CISH | GATA3 |
| CBLB | CISH | KDM1A |
| CBLB | CISH | MED12 |
| CBLB | CISH | MYB |
| CBLB | CISH | PRDM1 |
| CBLB | CISH | RASA2 |
| CBLB | CISH | TGFBR2 |
| CBLB | DGKZ | ELOB |
| CBLB | DGKZ | FAS |
| CBLB | DGKZ | Fli1 |
| CBLB | DGKZ | GATA3 |
| CBLB | DGKZ | KDM1A |
| CBLB | DGKZ | MED12 |
| CBLB | DGKZ | MYB |
| CBLB | DGKZ | PRDM1 |
| CBLB | DGKZ | RASA2 |
| CBLB | DGKZ | TGFBR2 |
| CBLB | ELOB | FAS |
| CBLB | ELOB | Fli1 |
| CBLB | ELOB | GATA3 |
| CBLB | ELOB | KDM1A |
| CBLB | ELOB | MED12 |
| CBLB | ELOB | MYB |
| CBLB | ELOB | PRDM1 |
| CBLB | ELOB | RASA2 |
| CBLB | ELOB | TGFBR2 |
| CBLB | FAS | Fli1 |
| CBLB | FAS | GATA3 |
| CBLB | FAS | KDM1A |
| CBLB | FAS | MED12 |
| CBLB | FAS | MYB |
| CBLB | FAS | PRDM1 |
| CBLB | FAS | RASA2 |
| CBLB | FAS | TGFBR2 |
| CBLB | Fli1 | GATA3 |
| CBLB | Fli1 | KDM1A |
| CBLB | Fli1 | MED12 |
| CBLB | Fli1 | MYB |
| CBLB | Fli1 | PRDM1 |
| CBLB | Fli1 | RASA2 |
| CBLB | Fli1 | TGFBR2 |
| CBLB | GATA3 | KDM1A |
| CBLB | GATA3 | MED12 |
| CBLB | GATA3 | MYB |
| CBLB | GATA3 | PRDM1 |
| CBLB | GATA3 | RASA2 |
| CBLB | GATA3 | TGFBR2 |
| CBLB | KDM1A | MED12 |
| CBLB | KDM1A | MYB |
| CBLB | KDM1A | PRDM1 |
| CBLB | KDMIA | RASA2 |
| CBLB | KDM1A | TGFBR2 |
| CBLB | MED12 | MYB |
| CBLB | MED12 | PRDM1 |
| CBLB | MED12 | RASA2 |
| CBLB | MED12 | TGFBR2 |
| CBLB | MYB | PRDM1 |
| CBLB | MYB | RASA2 |
| CBLB | MYB | TGFBR2 |
| CBLB | PRDM1 | RASA2 |
| CBLB | PRDM1 | TGFBR2 |
| CBLB | RASA2 | TGFBR2 |
| CCNC | CD5 | CISH |
| CCNC | CD5 | DGKZ |
| CCNC | CD5 | ELOB |
| CCNC | CD5 | FAS |
| CCNC | CD5 | Fli1 |
| CCNC | CD5 | GATA3 |
| CCNC | CD5 | KDM1A |
| CCNC | CD5 | MED12 |
| CCNC | CD5 | MYB |
| CCNC | CD5 | PRDM1 |
| CCNC | CD5 | RASA2 |
| CCNC | CD5 | TGFBR2 |
| CCNC | CISH | DGKZ |
| CCNC | CISH | ELOB |
| CCNC | CISH | FAS |
| CCNC | CISH | Fli1 |
| CCNC | CISH | GATA3 |
| CCNC | CISH | KDM1A |
| CCNC | CISH | MED12 |
| CCNC | CISH | MYB |
| CCNC | CISH | PRDM1 |
| CCNC | CISH | RASA2 |
| CCNC | CISH | TGFBR2 |
| CCNC | DGKZ | ELOB |
| CCNC | DGKZ | FAS |
| CCNC | DGKZ | Fli1 |
| CCNC | DGKZ | GATA3 |
| CCNC | DGKZ | KDM1A |
| CCNC | DGKZ | MED12 |
| CCNC | DGKZ | MYB |
| CCNC | DGKZ | PRDM1 |
| CCNC | DGKZ | RASA2 |
| CCNC | DGKZ | TGFBR2 |
| CCNC | ELOB | FAS |
| CCNC | ELOB | Fli1 |
| CCNC | ELOB | GATA3 |
| CCNC | ELOB | KDM1A |
| CCNC | ELOB | MED12 |
| CCNC | ELOB | MYB |
| CCNC | ELOB | PRDM1 |
| CCNC | ELOB | RASA2 |
| CCNC | ELOB | TGFBR2 |
| CCNC | FAS | Fli1 |
| CCNC | FAS | GATA3 |
| CCNC | FAS | KDM1A |
| CCNC | FAS | MED12 |
| CCNC | FAS | MYB |
| CCNC | FAS | PRDM1 |
| CCNC | FAS | RASA2 |
| CCNC | FAS | TGFBR2 |
| CCNC | Fli1 | GATA3 |
| CCNC | Fli1 | KDM1A |
| CCNC | Fli1 | MED12 |
| CCNC | Fli1 | MYB |
| CCNC | Fli1 | PRDM1 |
| CCNC | Fli1 | RASA2 |
| CCNC | Fli1 | TGFBR2 |
| CCNC | GATA3 | KDM1A |
| CCNC | GATA3 | MED12 |
| CCNC | GATA3 | MYB |
| CCNC | GATA3 | PRDM1 |
| CCNC | GATA3 | RASA2 |
| CCNC | GATA3 | TGFBR2 |
| CCNC | KDM1A | MED12 |
| CCNC | KDM1A | MYB |
| CCNC | KDM1A | PRDM1 |
| CCNC | KDM1A | RASA2 |
| CCNC | KDM1A | TGFBR2 |
| CCNC | MED12 | MYB |
| CCNC | MED12 | PRDM1 |
| CCNC | MED12 | RASA2 |
| CCNC | MED12 | TGFBR2 |
| CCNC | MYB | PRDM1 |
| CCNC | MYB | RASA2 |
| CCNC | MYB | TGFBR2 |
| CCNC | PRDM1 | RASA2 |
| CCNC | PRDM1 | TGFBR2 |
| CCNC | RASA2 | TGFBR2 |
| CD5 | CISH | DGKZ |
| CD5 | CISH | ELOB |
| CD5 | CISH | FAS |
| CD5 | CISH | Fli1 |
| CD5 | CISH | GATA3 |
| CD5 | CISH | KDM1A |
| CD5 | CISH | MED12 |
| CD5 | CISH | MYB |
| CD5 | CISH | PRDM1 |
| CD5 | CISH | RASA2 |
| CD5 | CISH | TGFBR2 |
| CD5 | DGKZ | ELOB |
| CD5 | DGKZ | FAS |
| CD5 | DGKZ | Fli1 |
| CD5 | DGKZ | GATA3 |
| CD5 | DGKZ | KDM1A |
| CD5 | DGKZ | MED12 |
| CD5 | DGKZ | MYB |
| CD5 | DGKZ | PRDM1 |
| CD5 | DGKZ | RASA2 |
| CD5 | DGKZ | TGFBR2 |
| CD5 | ELOB | FAS |
| CD5 | ELOB | Fli1 |
| CD5 | ELOB | GATA3 |
| CD5 | ELOB | KDM1A |
| CD5 | ELOB | MED12 |
| CD5 | ELOB | MYB |
| CD5 | ELOB | PRDM1 |
| CD5 | ELOB | RASA2 |
| CD5 | ELOB | TGFBR2 |
| CD5 | FAS | Fli1 |
| CD5 | FAS | GATA3 |
| CD5 | FAS | KDM1A |
| CD5 | FAS | MED12 |
| CD5 | FAS | MYB |
| CD5 | FAS | PRDM1 |
| CD5 | FAS | RASA2 |
| CD5 | FAS | TGFBR2 |
| CD5 | Fli1 | GATA3 |
| CD5 | Fli1 | KDM1A |
| CD5 | Fli1 | MED12 |
| CD5 | Fli1 | MYB |
| CD5 | Fli1 | PRDM1 |
| CD5 | Fli1 | RASA2 |
| CD5 | Fli1 | TGFBR2 |
| CD5 | GATA3 | KDM1A |
| CD5 | GATA3 | MED12 |
| CD5 | GATA3 | MYB |
| CD5 | GATA3 | PRDM1 |
| CD5 | GATA3 | RASA2 |
| CD5 | GATA3 | TGFBR2 |
| CD5 | KDM1A | MED12 |
| CD5 | KDM1A | MYB |
| CD5 | KDM1A | PRDM1 |
| CD5 | KDM1A | RASA2 |
| CD5 | KDM1A | TGFBR2 |
| CD5 | MED12 | MYB |
| CD5 | MED12 | PRDM1 |
| CD5 | MED12 | RASA2 |
| CD5 | MED12 | TGFBR2 |
| CD5 | MYB | PRDM1 |
| CD5 | MYB | RASA2 |
| CD5 | MYB | TGFBR2 |
| CD5 | PRDM1 | RASA2 |
| CD5 | PRDM1 | TGFBR2 |
| CD5 | RASA2 | TGFBR2 |
| CISH | DGKZ | ELOB |
| CISH | DGKZ | FAS |
| CISH | DGKZ | Fli1 |
| CISH | DGKZ | GATA3 |
| CISH | DGKZ | KDM1A |
| CISH | DGKZ | MED12 |
| CISH | DGKZ | MYB |
| CISH | DGKZ | PRDM1 |
| CISH | DGKZ | RASA2 |
| CISH | DGKZ | TGFBR2 |
| CISH | ELOB | FAS |
| CISH | ELOB | Fli1 |
| CISH | ELOB | GATA3 |
| CISH | ELOB | KDM1A |
| CISH | ELOB | MED12 |
| CISH | ELOB | MYB |
| CISH | ELOB | PRDM1 |
| CISH | ELOB | RASA2 |
| CISH | ELOB | TGFBR2 |
| CISH | FAS | Fli1 |
| CISH | FAS | GATA3 |
| CISH | FAS | KDM1A |
| CISH | FAS | MED12 |
| CISH | FAS | MYB |
| CISH | FAS | PRDM1 |
| CISH | FAS | RASA2 |
| CISH | FAS | TGFBR2 |
| CISH | Fli1 | GATA3 |
| CISH | Fli1 | KDM1A |
| CISH | Fli1 | MED12 |
| CISH | Fli1 | MYB |
| CISH | Fli1 | PRDM1 |
| CISH | Fli1 | RASA2 |
| CISH | Fli1 | TGFBR2 |
| CISH | GATA3 | KDM1A |
| CISH | GATA3 | MED12 |
| CISH | GATA3 | MYB |
| CISH | GATA3 | PRDM1 |
| CISH | GATA3 | RASA2 |
| CISH | GATA3 | TGFBR2 |
| CISH | KDM1A | MED12 |
| CISH | KDM1A | MYB |
| CISH | KDM1A | PRDM1 |
| CISH | KDM1A | RASA2 |
| CISH | KDM1A | TGFBR2 |
| CISH | MED12 | MYB |
| CISH | MED12 | PRDM1 |
| CISH | MED12 | RASA2 |
| CISH | MED12 | TGFBR2 |
| CISH | MYB | PRDM1 |
| CISH | MYB | RASA2 |
| CISH | MYB | TGFBR2 |
| CISH | PRDM1 | RASA2 |
| CISH | PRDM1 | TGFBR2 |
| CISH | RASA2 | TGFBR2 |
| DGKZ | ELOB | FAS |
| DGKZ | ELOB | Fli1 |
| DGKZ | ELOB | GATA3 |
| DGKZ | ELOB | KDM1A |
| DGKZ | ELOB | MED12 |
| DGKZ | ELOB | MYB |
| DGKZ | ELOB | PRDM1 |
| DGKZ | ELOB | RASA2 |
| DGKZ | ELOB | TGFBR2 |
| DGKZ | FAS | Fli1 |
| DGKZ | FAS | GATA3 |
| DGKZ | FAS | KDM1A |
| DGKZ | FAS | MED12 |
| DGKZ | FAS | MYB |
| DGKZ | FAS | PRDM1 |
| DGKZ | FAS | RASA2 |
| DGKZ | FAS | TGFBR2 |
| DGKZ | Fli1 | GATA3 |
| DGKZ | Fli1 | KDM1A |
| DGKZ | Fli1 | MED12 |
| DGKZ | Fli1 | MYB |
| DGKZ | Fli1 | PRDM1 |
| DGKZ | Fli1 | RASA2 |
| DGKZ | Fli1 | TGFBR2 |
| DGKZ | GATA3 | KDM1A |
| DGKZ | GATA3 | MED12 |
| DGKZ | GATA3 | MYB |
| DGKZ | GATA3 | PRDM1 |
| DGKZ | GATA3 | RASA2 |
| DGKZ | GATA3 | TGFBR2 |
| DGKZ | KDM1A | MED12 |
| DGKZ | KDM1A | MYB |
| DGKZ | KDM1A | PRDM1 |
| DGKZ | KDM1A | RASA2 |
| DGKZ | KDM1A | TGFBR2 |
| DGKZ | MED12 | MYB |
| DGKZ | MED12 | PRDM1 |
| DGKZ | MED12 | RASA2 |
| DGKZ | MED12 | TGFBR2 |
| DGKZ | MYB | PRDM1 |
| DGKZ | MYB | RASA2 |
| DGKZ | MYB | TGFBR2 |
| DGKZ | PRDM1 | RASA2 |
| DGKZ | PRDM1 | TGFBR2 |
| DGKZ | RASA2 | TGFBR2 |
| ELOB | FAS | Fli1 |
| ELOB | FAS | GATA3 |
| ELOB | FAS | KDM1A |
| ELOB | FAS | MED12 |
| ELOB | FAS | MYB |
| ELOB | FAS | PRDM1 |
| ELOB | FAS | RASA2 |
| ELOB | FAS | TGFBR2 |
| ELOB | Fli1 | GATA3 |
| ELOB | Fli1 | KDM1A |
| ELOB | Fli1 | MED12 |
| ELOB | Fli1 | MYB |
| ELOB | Fli1 | PRDM1 |
| ELOB | Fli1 | RASA2 |
| ELOB | Fli1 | TGFBR2 |
| ELOB | GATA3 | KDM1A |
| ELOB | GATA3 | MED12 |
| ELOB | GATA3 | MYB |
| ELOB | GATA3 | PRDM1 |
| ELOB | GATA3 | RASA2 |
| ELOB | GATA3 | TGFBR2 |
| ELOB | KDM1A | MED12 |
| ELOB | KDM1A | MYB |
| ELOB | KDM1A | PRDM1 |
| ELOB | KDM1A | RASA2 |
| ELOB | KDM1A | TGFBR2 |
| ELOB | MED12 | MYB |
| ELOB | MED12 | PRDM1 |
| ELOB | MED12 | RASA2 |
| ELOB | MED12 | TGFBR2 |
| ELOB | MYB | PRDM1 |
| ELOB | MYB | RASA2 |
| ELOB | MYB | TGFBR2 |
| ELOB | PRDM1 | RASA2 |
| ELOB | PRDM1 | TGFBR2 |
| ELOB | RASA2 | TGFBR2 |
| FAS | Fli1 | GATA3 |
| FAS | Fli1 | KDM1A |
| FAS | Fli1 | MED12 |
| FAS | Fli1 | MYB |
| FAS | Fli1 | PRDM1 |
| FAS | Fli1 | RASA2 |
| FAS | Fli1 | TGFBR2 |
| FAS | GATA3 | KDM1A |
| FAS | GATA3 | MED12 |
| FAS | GATA3 | MYB |
| FAS | GATA3 | PRDM1 |
| FAS | GATA3 | RASA2 |
| FAS | GATA3 | TGFBR2 |
| FAS | KDM1A | MED12 |
| FAS | KDM1A | MYB |
| FAS | KDM1A | PRDM1 |
| FAS | KDM1A | RASA2 |
| FAS | KDM1A | TGFBR2 |
| FAS | MED12 | MYB |
| FAS | MED12 | PRDM1 |
| FAS | MED12 | RASA2 |
| FAS | MED12 | TGFBR2 |
| FAS | MYB | PRDM1 |
| FAS | MYB | RASA2 |
| FAS | MYB | TGFBR2 |
| FAS | PRDM1 | RASA2 |
| FAS | PRDM1 | TGFBR2 |
| FAS | RASA2 | TGFBR2 |
| Fli1 | GATA3 | KDM1A |
| Fli1 | GATA3 | MED12 |
| Fli1 | GATA3 | MYB |
| Fli1 | GATA3 | PRDM1 |
| Fli1 | GATA3 | RASA2 |
| Fli1 | GATA3 | TGFBR2 |
| Fli1 | KDM1A | MED12 |
| Fli1 | KDM1A | MYB |
| Fli1 | KDM1A | PRDM1 |
| Fli1 | KDM1A | RASA2 |
| Fli1 | KDM1A | TGFBR2 |
| Fli1 | MED12 | MYB |
| Fli1 | MED12 | PRDM1 |
| Fli1 | MED12 | RASA2 |
| Fli1 | MED12 | TGFBR2 |
| Fli1 | MYB | PRDM1 |
| Fli1 | MYB | RASA2 |
| Fli1 | MYB | TGFBR2 |
| Fli1 | PRDM1 | RASA2 |
| Fli1 | PRDM1 | TGFBR2 |
| Fli1 | RASA2 | TGFBR2 |
| GATA3 | KDM1A | MED12 |
| GATA3 | KDM1A | MYB |
| GATA3 | KDM1A | PRDM1 |
| GATA3 | KDM1A | RASA2 |
| GATA3 | KDM1A | TGFBR2 |
| GATA3 | MED12 | MYB |
| GATA3 | MED12 | PRDM1 |
| GATA3 | MED12 | RASA2 |
| GATA3 | MED12 | TGFBR2 |
| GATA3 | MYB | PRDM1 |
| GATA3 | MYB | RASA2 |
| GATA3 | MYB | TGFBR2 |
| GATA3 | PRDM1 | RASA2 |
| GATA3 | PRDM1 | TGFBR2 |
| GATA3 | RASA2 | TGFBR2 |
| KDM1A | MED12 | MYB |
| KDM1A | MED12 | PRDM1 |
| KDM1A | MED12 | RASA2 |
| KDM1A | MED12 | TGFBR2 |
| KDM1A | MYB | PRDM1 |
| KDM1A | MYB | RASA2 |
| KDM1A | MYB | TGFBR2 |
| KDM1A | PRDM1 | RASA2 |
| KDM1A | PRDM1 | TGFBR2 |
| KDM1A | RASA2 | TGFBR2 |
| MED12 | MYB | PRDM1 |
| MED12 | MYB | RASA2 |
| MED12 | MYB | TGFBR2 |
| MED12 | PRDM1 | RASA2 |
| MED12 | PRDM1 | TGFBR2 |
| MED12 | RASA2 | TGFBR2 |
| MYB | PRDM1 | RASA2 |
| MYB | PRDM1 | TGFBR2 |
| MYB | RASA2 | TGFBR2 |
| PRDM1 | RASA2 | TGFBR2 |

In some embodiments, the DNA-targeting system targets a target site for CBLB, CCNC, CD5, CISH, DGKZ, ELOB, FAS, Fli1, GATA3, KDM1A, MED12, MYB, PRDM1, TGFBR2, and/or RASA2. In some embodiments, the target site comprises a sequence selected from any one of SEQ ID NOS: 1-6, 10-33, 80-90, 102-112, 200-211, 292-295, 300-302, and 306-308, or a contiguous portion thereof of at least 14 nucleotides, or a complementary sequence of any of the foregoing. In some embodiments, the target site is a contiguous portion of any one of SEQ ID NOS: 1-6, 10-33, 80-90, 102-112, 200-211, 292-295, 300-302, and 306-308 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 1-6, 10-33, 80-90, 102-112, 200-211, 292-295, 300-302, and 306-308. In some embodiments, the target site is a sequence set forth in Table 5.

In some embodiments, the DNA-targeting system targets a target site for CBLB, CISH, MED12, MYB, PRDM1, and/or RASA2.

In some embodiments, the DNA-targeting system targets a target site for CBLB. In some embodiments, the target site for regulating transcription of CBLB is within the coordinates chr3: 105868857-105868876. In some embodiments, the target site is within the coordinates chr3: 105868757-105868976. In some embodiments, the target site is within the coordinates chr3: 105868807-105868926. In some embodiments, the target site is within the coordinates chr3: 105868837-105868896. In some embodiments, the target site is or includes the coordinates chr3: 105868857-105868876. In some embodiments the target site for CBLB is located within 500 bp of human genome assembly GRCh38 (hg38) genomic coordinates chr3: 105,655,461 (e.g., a target site that is +500 of 105,655,461 or −500 of 105,655,461 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr3: 105,655,461. In some embodiments the target site is located within about 80 bp of the genomic coordinate chr3: 105,655,461. In some embodiments, the target site in the region from −40 to +40 of the genomic coordinate chr3: 105,655,461. In some embodiments the target site is located within 20 bp of the genomic coordinate chr3: 105,655,461. In some embodiments, the gRNA targets a target site in the region from −10 to +10 of the genomic coordinate chr3: 105,655,461. In some embodiments, any of such target sites include or span the genomic coordinate chr3: 105,655,461, which is a CBLB transcription start site (TSS). In some embodiments, the target site comprises a sequence set forth in SEQ ID NO: 11, or a contiguous portion thereof of at least 14 nucleotides, or a complementary sequence of any of the foregoing. In some embodiments, the target site is a contiguous portion of SEQ ID NO:11 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:11.

In some embodiments, the DNA-targeting system targets a target site for CISH. In some embodiments, the target site for regulating transcription of CISH is within the coordinates chr3: 50,611,749-50,611,768. In some embodiments, the target site is within the coordinates chr3: 50,611,649-50611868. In some embodiments, the target site is within the coordinates chr3: 50,611,699-50611818. In some embodiments, the target site is within the coordinates chr3: 50,611,729-50611788. In some embodiments, the target site is or includes the coordinates chr1: 50,611,749-50,611,768. In some embodiments the target site for CISH is located within 500 bp of human genome assembly GRCh38 (hg38) genomic coordinates chr3: 50,606,489 (e.g., a target site that is +500 of 50,606,489 or −500 of 50,606,489 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr3: 50,606,489. In some embodiments the target site is located within about 80 bp of the genomic coordinate chr3: 50,606,489. In some embodiments, the target site in the region from −40 to +40 of the genomic coordinate chr3: 50,606,489. In some embodiments the target site is located within 20 bp of the genomic coordinate chr3: 50,606,489. In some embodiments, the gRNA targets a target site in the region from −10 to +10 of the genomic coordinate chr3: 50,606,489. In some embodiments, any of such target sites include or span the genomic coordinate chr3: 50,606,489, which is a CISH transcription start site (TSS). In some embodiments, the target site comprises a sequence set forth in SEQ ID NO: 28, or a contiguous portion thereof of at least 14 nucleotides, or a complementary sequence of any of the foregoing. In some embodiments, the target site is a contiguous portion of SEQ ID NO:28 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:28.

In some embodiments, the DNA-targeting system targets a target site for MED12. In some embodiments, the target site for regulating transcription of MED12 is within the coordinates chrX: 71,118,489-71,118,508. In some embodiments, the target site is within the coordinates chrX: 71,118,389-71,118,608. In some embodiments, the target site is within the coordinates chrX: 71,118,439-71,118,558. In some embodiments, the target site is within the coordinates chrX: 71,118,469-71,118,528. In some embodiments, the target site is or includes the coordinates chrX: 71,118,489-71,118,508. In some embodiments the target site for MED12 is located within 500 bp of human genome assembly GRCh38 (hg38) genomic coordinates chrX: 71,118,596 (e.g., a target site that is +500 of 71,118,596 or −500 of 71,118,596 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr3: 50,606,489. In some embodiments the target site is located within about 80 bp of the genomic coordinate chrX: 71,118,596. In some embodiments, the target site in the region from −40 to +40 of the genomic coordinate 71,118,596. In some embodiments the target site is located within 20 bp of the genomic coordinate chrX: 71,118,596. In some embodiments, the gRNA targets a target site in the region from −10 to +10 of the genomic coordinate chrX: 71,118,596. In some embodiments, any of such target sites include or span the genomic coordinate chrX: 71,118,596, which is a MED12 transcription start site (TSS). In some embodiments, the target site comprises a sequence set forth in SEQ ID NO: 81, or a contiguous portion thereof of at least 14 nucleotides, or a complementary sequence of any of the foregoing. In some embodiments, the target site is a contiguous portion of SEQ ID NO:81 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:81.

In some embodiments, the DNA-targeting system targets a target site for MYB. In some embodiments, the target site for regulating the transcription of MYB is within the coordinates chr6:135,181,383-135,181,402. In some embodiments, the target site is within the coordinates chr6: 135,181,283-135,181,502. In some embodiments, the target site is within the coordinates chr6: 135,181,333-135,181,452. In some embodiments, the target site is within the coordinates chr6: 135,181,363-135,181,422. In some embodiments, the target site is or includes the coordinates chr6:135,181,383-135,181,402. In some embodiments the target site for MYB is located within 500 bp of human genome assembly GRCh38 (hg38) genomic coordinates chr6: 135,181,308 (e.g., a target site that is +500 of 135,181,308 or −500 of 135,181,308 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr6: 135,181,308. In some embodiments the target site is located within about 80 bp of the genomic coordinate chr6: 135,181,308. In some embodiments, the target site in the region from −40 to +40 of the genomic coordinate 135,181,308. In some embodiments the target site is located within 20 bp of the genomic coordinate chr6: 135,181,308. In some embodiments, the gRNA targets a target site in the region from −10 to +10 of the genomic coordinate chr6: 135,181,308. In some embodiments, any of such target sites include or span the genomic coordinate chr6: 135,181,308, which is a MYB transcription start site (TSS). In some embodiments, the target site comprises a sequence set forth in SEQ ID NO: 18, or a contiguous portion thereof of at least 14 nucleotides, or a complementary sequence of any of the foregoing. In some embodiments, the target site is a contiguous portion of SEQ ID NO:18 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:18.

In some embodiments, the DNA-targeting system targets a target site for RASA2. In some embodiments, the target site for regulating transcription of RASA2 is within the coordinates chr3:141,487,065-141,487,084. In some embodiments, the target site is within the coordinates chr3: 141,486,965-141,487,184. In some embodiments, the target site is within the coordinates chr3: 141,487,015-141,487,134. In some embodiments, the target site is within the coordinates chr3: 141,487,045-141,487,104. In some embodiments, the target site is or includes the coordinates chr3:141,487,065-141,487,084. In some embodiments the target site for RASA2 is located within 500 bp of human genome assembly GRCh38 (hg38) genomic coordinates chr3: 141,487,027 (e.g., a target site that is +500 of 141,487,027 or −500 of 141,487,027 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr3: 141,487,027. In some embodiments the target site is located within about 80 bp of the genomic coordinate chr3: 141,487,027. In some embodiments, the target site in the region from −40 to +40 of the genomic coordinate 141,487,027. In some embodiments the target site is located within 20 bp of the genomic coordinate chr3: 141,487,027. In some embodiments, the gRNA targets a target site in the region from −10 to +10 of the genomic coordinate chr3: 141,487,027. In some embodiments, any of such target sites include or span the genomic coordinate chr3: 141,487,027, which is a RASA2 transcription start site (TSS). In some embodiments, the target site comprises a sequence set forth in SEQ ID NO: 19, or a contiguous portion thereof of at least 14 nucleotides, or a complementary sequence of any of the foregoing. In some embodiments, the target site is a contiguous portion of SEQ ID NO:19 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:19.

In some embodiments, the DNA-targeting system targets a target site for PRDM1. In some embodiments, the target site for regulating transcription of PRDM1 is within the coordinates chr6:106,086,371-106,086,390. In some embodiments, the target site is within the coordinates chr6: 106,086,271-106,086,490. In some embodiments, the target site is within the coordinates chr6: 106,086,321-106,086,440. In some embodiments, the target site is within the coordinates chr6: 106,086,351-106,086,410. In some embodiments, the target site is or includes the coordinates chr6:106,086,371-106,086,390. In some embodiments the target site for PRDM1 is located within 500 bp of human genome assembly GRCh38 (hg38) genomic coordinates chr6: 106,086,336 (e.g., a target site that is +500 of 106,086,336 or −500 of 106,086,336 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr6: 106,086,336. In some embodiments the target site is located within about 80 bp of the genomic coordinate chr6: 106,086,336. In some embodiments, the target site in the region from −40 to +40 of the genomic coordinate 106,086,336. In some embodiments the target site is located within 20 bp of the genomic coordinate chr6: 106,086,336. In some embodiments, the gRNA targets a target site in the region from −10 to +10 of the genomic coordinate chr6: 106,086,336. In some embodiments, any of such target sites include or span the genomic coordinate chr6: 106,086,336, which is a PRDM1 transcription start site (TSS). In some embodiments, the target site comprises a sequence set forth in SEQ ID NO: 33, or a contiguous portion thereof of at least 14 nucleotides, or a complementary sequence of any of the foregoing. In some embodiments, the target site is a contiguous portion of SEQ ID NO:33 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:33.

In some embodiments, the DNA-targeting system targets a combination of genes, such as any combination shown in Table 1. In some embodiments, the DNA-targeting system targets a combination of genes selected from: CBLB and CCNC; CBLB and CD5; CBLB and CISH; CBLB and DGKZ; CBLB and ELOB; CBLB and FAS; CBLB and Fli1; CBLB and GATA3; CBLB and KDM1A; CBLB and MED12; CBLB and MYB; CBLB and PRDM1; CBLB and RASA2; CD5 and CISH; CD5 and MYB; CISH and DGKZ; CISH and MYB; CISH and RASA2; GATA3 and CD5; GATA3 and CISH; GATA3 and MYB; MED12 and CBLB; MED12 and CD5; MED12 and CISH; MED12 and DGKZ; MED12 and ELOB; MED12 and GATA3; MED12 and MYB; MED12 and PRDM1; MED12 and RASA2; MYB and RASA2; PRDM1 and CISH; PRDM1 and GATA3; PRDM1 and MYB; PRDM1 and RASA2; CD5, CISH, and MYB; GATA3, CBLB, and MYB; GATA3, CD5, and MYB; PRDM1, GATA3, and CISH; TGBR2 and MED12; and TGFBR2, MED12, and CISH.

In some embodiments, the DNA-targeting system targets CBLB and MYB. In some embodiments, the target site for targeting CBLB can be any as described above, and the target site for targeting MYB can be any as described above. In some embodiments, the DNA-targeting system targets a target site for CBLB comprising the sequence set forth in SEQ ID NO:11, and a target site for MYB comprising the sequence set forth in SEQ ID NO:18.

In some embodiments, the DNA-targeting system targets CBLB and MED12. In some embodiments, the target site for targeting CBLB can be any as described above, and the target site for targeting MED12 can be any as described above. In some embodiments, the DNA-targeting system targets a target site for CBLB comprising the sequence set forth in SEQ ID NO:11, and a target site for MED12 comprising the sequence set forth in SEQ ID NO:81.

In some embodiments, the DNA-targeting system targets CBLB and CCNC. In some embodiments, the target site for targeting CBLB can be any as described above, and the target site for targeting CCNC can be any as described above. In some embodiments, the DNA-targeting system targets a target site for CBLB comprising the sequence set forth in SEQ ID NO:11, and a target site for CCNC comprising the sequence set forth in SEQ ID NO:104.

In some embodiments, the DNA-targeting system targets MED12 and CISH. In some embodiments, the target site for targeting MED12 can be any as described above, and the target site for targeting CISH can be any as described above. In some embodiments, the DNA-targeting system targets a target site for MED12 comprising the sequence set forth in SEQ ID NO:81, and a target site for CISH comprising the sequence set forth in SEQ ID NO:28.

In some embodiments, the DNA-targeting system targets MED12, CBLB and CISH. In some embodiments, the target site for targeting MED12 can be any as described above, the target site for targeting CBLB can be any as described above, and the target site for targeting CISH can be any as described above. In some embodiments, the DNA-targeting system targets a target site for MED12 comprising the sequence set forth in SEQ ID NO:81, a target site for CBLB comprising the sequence set forth in SEQ ID NO:11, and a target site for CISH comprising the sequence set forth in SEQ ID NO:28.

In some embodiments, the DNA-targeting system targets MED12 and RASA2. In some embodiments, the target site for targeting MED12 can be any as described above, and the target site for targeting RASA2 can be any as described above. In some embodiments, the DNA-targeting system targets a target site for MED12 comprising the sequence set forth in SEQ ID NO:81, and a target site for RASA2 comprising the sequence set forth in SEQ ID NO:19.

In some embodiments, the DNA-targeting system targets TGFBR2 and MED12. In some embodiments, the DNA-targeting system targets a target site for TGBR2 comprising the sequence set forth in SEQ ID NO:301, and a target site for MED12 comprising the sequence set forth in SEQ ID NO:82.

In some embodiments, the DNA-targeting system targets TGFBR2, MED12, and CISH. In some embodiments, the DNA-targeting system targets a target site for TGBR2 comprising the sequence set forth in SEQ ID NO:301, a target site for MED12 comprising the sequence set forth in SEQ ID NO:82, and a target site for CISH comprising the sequence set forth in SEQ ID NO:28.

In some embodiments, the DNA-targeting system targets a combination of target sites for a combination of genes for transcriptional repression, as shown in Table 2.

TABLE 2
Gene and target site combinations for transcriptional repression

| | Target Site 1 | | Target Site 2 | | Target Gene 3 |
|---|---|---|---|---|---|
| Target Gene 1 | SEQ ID NO: | Target Gene 2 | SEQ ID NO: | Target Gene 3 | SEQ ID NO: |
| CBLB | 11 | CCNC | 104 | — | — |
| CBLB | 11 | CD5 | 3 | — | — |
| CBLB | 11 | CISH | 30 | — | — |
| CBLB | 11 | DGKZ | 13 | — | — |
| CBLB | 11 | ELOB | 24 | — | — |
| CBLB | 11 | FAS | 204 | — | — |
| CBLB | 11 | Fli1 | 208 | — | — |
| CBLB | 11 | GATA3 | 26 | — | — |
| CBLB | 11 | KDM1A | 4 | — | — |
| CBLB | 11 | MED12 | 81 | — | — |
| CBLB | 11 | MYB | 18 | — | — |
| CBLB | 11 | PRDM1 | 32 | — | — |
| CBLB | 11 | RASA2 | 19 | — | — |
| CD5 | 3 | CISH | 30 | — | — |
| CD5 | 3 | MYB | 18 | — | — |
| CISH | 30 | DGKZ | 13 | — | — |
| CISH | 30 | MYB | 18 | — | — |
| CISH | 30 | RASA2 | 19 | — | — |
| GATA3 | 26 | CD5 | 3 | — | — |
| GATA3 | 26 | CISH | 30 | — | — |
| GATA3 | 26 | MYB | 18 | — | — |
| MED12 | 81 | CBLB | 11 | — | — |
| MED12 | 81 | CD5 | 3 | — | — |
| MED12 | 81 | CISH | 30 | — | — |
| MED12 | 81 | DGKZ | 13 | — | — |
| MED12 | 81 | ELOB | 24 | — | — |
| MED12 | 81 | GATA3 | 26 | — | — |
| MED12 | 81 | MYB | 18 | — | — |
| MED12 | 81 | PRDM1 | 32 | — | — |
| MED12 | 81 | RASA2 | 19 | — | — |
| MYB | 18 | RASA2 | 19 | — | — |
| PRDM1 | 32 | CISH | 30 | — | — |
| PRDM1 | 32 | GATA3 | 26 | — | — |
| PRDM1 | 32 | MYB | 18 | — | — |
| PRDM1 | 32 | RASA2 | 19 | — | — |
| TGFBR2 | 301 | MED12 | 82 | | |
| CD5 | 3 | CISH | 30 | MYB | 18 |
| GATA3 | 26 | CBLB | 11 | MYB | 18 |
| GATA3 | 26 | CD5 | 3 | MYB | 18 |
| PRDM1 | 32 | GATA3 | 26 | CISH | 30 |
| TGFBR2 | 301 | MED12 | 82 | CISH | 28 |

In some embodiments, the DNA-targeting system targets a target site for CBLB comprising the sequence set forth in SEQ ID NO: 11, and a target site for CCNC comprising the sequence set forth in SEQ ID NO: 104. In some embodiments, the DNA-targeting system targets a target site for CBLB comprising the sequence set forth in SEQ ID NO: 11, and a target site for CD5 comprising the sequence set forth in SEQ ID NO:3. In some embodiments, the DNA-targeting system targets a target site for CB LB comprising the sequence set forth in SEQ ID NO: 11, and a target site for CISH comprising the sequence set forth in SEQ ID NO:30. In some embodiments, the DNA-targeting system targets a target site for CBLB comprising the sequence set forth in SEQ ID NO: 11, and a target site for DGKZ comprising the sequence set forth in SEQ ID NO: 13. In some embodiments, the DNA-targeting system targets a target site for CBLB comprising the sequence set forth in SEQ ID NO: 11, and a target site for ELOB comprising the sequence set forth in SEQ ID NO:24. In some embodiments, the DNA-targeting system targets a target site for CBLB comprising the sequence set forth in SEQ ID NO:11, and a target site for FAS comprising the sequence set forth in SEQ ID NO:204. In some embodiments, the DNA-targeting system targets a target site for CBLB comprising the sequence set forth in SEQ ID NO:11, and a target site for Fli1 comprising the sequence set forth in SEQ ID NO:208. In some embodiments, the DNA-targeting system targets a target site for CBLB comprising the sequence set forth in SEQ ID NO:11, and a target site for GATA3 comprising the sequence set forth in SEQ ID NO:26. In some embodiments, the DNA-targeting system targets a target site for CBLB comprising the sequence set forth in SEQ ID NO:11, and a target site for KDM1A comprising the sequence set forth in SEQ ID NO:4. In some embodiments, the DNA-targeting system targets a target site for CBLB comprising the sequence set forth in SEQ ID NO:11, and a target site for MED12 comprising the sequence set forth in SEQ ID NO:81. In some embodiments, the DNA-targeting system targets a target site for CBLB comprising the sequence set forth in SEQ ID NO:11, and a target site for MYB comprising the sequence set forth in SEQ ID NO:18. In some embodiments, the DNA-targeting system targets a target site for CBLB comprising the sequence set forth in SEQ ID NO:11, and a target site for PRDM1 comprising the sequence set forth in SEQ ID NO:32. In some embodiments, the DNA-targeting system targets a target site for CBLB comprising the sequence set forth in SEQ ID NO:11, and a target site for RASA2 comprising the sequence set forth in SEQ ID NO:19. In some embodiments, the DNA-targeting system targets a target site for CD5 comprising the sequence set forth in SEQ ID NO:3, and a target site for CISH comprising the sequence set forth in SEQ ID NO:30. In some embodiments, the DNA-targeting system targets a target site for CD5 comprising the sequence set forth in SEQ ID NO:3, and a target site for MYB comprising the sequence set forth in SEQ ID NO:18. In some embodiments, the DNA-targeting system targets a target site for CISH comprising the sequence set forth in SEQ ID NO:30, and a target site for DGKZ comprising the sequence set forth in SEQ ID NO:13. In some embodiments, the DNA-targeting system targets a target site for CISH comprising the sequence set forth in SEQ ID NO:30, and a target site for MYB comprising the sequence set forth in SEQ ID NO:18. In some embodiments, the DNA-targeting system targets a target site for CISH comprising the sequence set forth in SEQ ID NO:30, and a target site for RASA2 comprising the sequence set forth in SEQ ID NO:19. In some embodiments, the DNA-targeting system targets a target site for GATA3 comprising the sequence set forth in SEQ ID NO:26, and a target site for CD5 comprising the sequence set forth in SEQ ID NO:3. In some embodiments, the DNA-targeting system targets a target site for GATA3 comprising the sequence set forth in SEQ ID NO:26, and a target site for CISH comprising the sequence set forth in SEQ ID NO:30. In some embodiments, the DNA-targeting system targets a target site for GATA3 comprising the sequence set forth in SEQ ID NO:26, and a target site for MYB comprising the sequence set forth in SEQ ID NO:18. In some embodiments, the DNA-targeting system targets a target site for MED12 comprising the sequence set forth in SEQ ID NO:81, and a target site for CBLB comprising the sequence set forth in SEQ ID NO:11. In some embodiments, the DNA-targeting system targets a target site for MED12 comprising the sequence set forth in SEQ ID NO:81, and a target site for CD5 comprising the sequence set forth in SEQ ID NO:3. In some embodiments, the DNA-targeting system targets a target site for MED12 comprising the sequence set forth in SEQ ID NO:81, and a target site for CISH comprising the sequence set forth in SEQ ID NO:30. In some embodiments, the DNA-targeting system targets a target site for MED12 comprising the sequence set forth in SEQ ID NO:81, and a target site for DGKZ comprising the sequence set forth in SEQ ID NO:13. In some embodiments, the DNA-targeting system targets a target site for MED12 comprising the sequence set forth in SEQ ID NO:81, and a target site for ELOB comprising the sequence set forth in SEQ ID NO:24. In some embodiments, the DNA-targeting system targets a target site for MED12 comprising the sequence set forth in SEQ ID NO:81, and a target site for GATA3 comprising the sequence set forth in SEQ ID NO:26. In some embodiments, the DNA-targeting system targets a target site for MED12 comprising the sequence set forth in SEQ ID NO:81, and a target site for MYB comprising the sequence set forth in SEQ ID NO:18. In some embodiments, the DNA-targeting system targets a target site for MED12 comprising the sequence set forth in SEQ ID NO:81, and a target site for PRDM1 comprising the sequence set forth in SEQ ID NO:32. In some embodiments, the DNA-targeting system targets a target site for MED12 comprising the sequence set forth in SEQ ID NO:81, and a target site for RASA2 comprising the sequence set forth in SEQ ID NO:19. In some embodiments, the DNA-targeting system targets a target site for MYB comprising the sequence set forth in SEQ ID NO:18, and a target site for RASA2 comprising the sequence set forth in SEQ ID NO:19. In some embodiments, the DNA-targeting system targets a target site for PRDM1 comprising the sequence set forth in SEQ ID NO:32, and a target site for CISH comprising the sequence set forth in SEQ ID NO:30. In some embodiments, the DNA-targeting system targets a target site for PRDM1 comprising the sequence set forth in SEQ ID NO:32, and a target site for GATA3 comprising the sequence set forth in SEQ ID NO:26. In some embodiments, the DNA-targeting system targets a target site for PRDM1 comprising the sequence set forth in SEQ ID NO:32, and a target site for MYB comprising the sequence set forth in SEQ ID NO:18. In some embodiments, the DNA-targeting system targets a target site for PRDM1 comprising the sequence set forth in SEQ ID NO:32, and a target site for RASA2 comprising the sequence set forth in SEQ ID NO:19. In some embodiments, the DNA-targeting system targets a target site for CD5 comprising the sequence set forth in SEQ ID NO:3, a target site for CISH comprising the sequence set forth in SEQ ID NO:30, and a target site for MYB comprising the sequence set forth in SEQ ID NO:18. In some embodiments, the DNA-targeting system targets a target site for GATA3 comprising the sequence set forth in SEQ ID NO:26, a target site for CBLB comprising the sequence set forth in SEQ ID NO:11, and a target site for MYB comprising the sequence set forth in SEQ ID NO:18. In some embodiments, the DNA-targeting system targets a target site for GATA3 comprising the sequence set forth in SEQ ID NO:26, a target site for CD5 comprising the sequence set forth in SEQ ID NO:3, and a target site for MYB comprising the sequence set forth in SEQ ID NO:18. In some embodiments, the DNA-targeting system targets a target site for PRDM1 comprising the sequence set forth in SEQ ID NO:32, a target site for GATA3 comprising the sequence set forth in SEQ ID NO:26, and a target site for CISH comprising the sequence set forth in SEQ ID NO:30.

In some embodiments, delivery of the DNA-targeting system reduces (e.g. decreases or represses) transcription of one or more genes. In some embodiments, the reduction in gene expression in a cell (e.g. T cell) is about a log 2 fold change of less than −1.0. For instance, the log 2 fold change is less than at or about −1.5, at or about −2.0, at or about −2.5, at or about −3.0, at or about −4.0, at or about −5.0, at or about −6.0, at or about −7.0, at or about −8.0, at or about −9.0, at or about −10.0 or any value between any of the foregoing compared to the level of the gene in a control cell.

### 3. Genes and Target Sites for Increasing Transcription

In some embodiments, delivery of the DNA-targeting system increases transcription of one or more genes selected from the group consisting of: BATF, CD28, EOMES, IL-2, IL2RB, IRF4, LAT, LCP2, TBX21, and VAV1. In some embodiments, provided herein are target sites for one or more genes for which increased transcription promotes a phenotype in a cell. In some embodiments, provided herein are target sites for one or more genes for which increased transcription promotes increased T cell effector function. In some embodiments, the increased transcription promotes increased T cell effector function upon T cell stimulation. In some embodiments, the one or more genes are selected from BATF, CD28, EOMES, IL-2, IL2RB, IRF4, LAT, LCP2, TBX21, and VAV1. In some embodiments, the one or more genes are selected from EOMES, IL-2, LCP2, and TBX21.

In some embodiments, the DNA-targeting system comprises a plurality of DNA-targeting modules. In some embodiments, each DNA-targeting module targets a target site. In some embodiments, the plurality of DNA-targeting modules target at least a first gene and a second gene, wherein the first and second gene are independently selected from the group consisting of BATF, CD28, EOMES, IL-2, IL2RB, IRF4, LAT, LCP2, TBX21, and VAV1. In some embodiments, the plurality of DNA-targeting modules target a first gene and a second gene, wherein the first and second gene are independently selected from the group consisting of BATF, CD28, EOMES, IL-2, IL2RB, IRF4, LAT, LCP2, TBX21, and VAV1. In some embodiments, the plurality of DNA-targeting modules target a first gene and a second gene, wherein the first and second gene are independently selected from the group consisting of EOMES, IL-2, LCP2, and TBX21.

In some embodiments, the plurality of DNA-targeting modules target at least a first gene, a second gene, and a third gene, wherein the first, second and third gene are independently selected from the group consisting of BATF, CD28, EOMES, IL-2, IL2RB, IRF4, LAT, LCP2, TBX21, and VAV1. In some embodiments, the plurality of DNA-targeting modules target a first gene, a second gene, and a third gene, wherein the first, second and third gene are independently selected from the group consisting of BATF, CD28, EOMES, IL-2, IL2RB, IRF4, LAT, LCP2, TBX21, and VAV1.

In some embodiments, the DNA-targeting system targets a combination of genes set forth in Table 3. In some embodiments, the plurality of DNA-targeting modules target a combination of genes set forth in Table 3. In some embodiments, transcription of each of the genes of the combination is increased by the DNA-targeting system.

TABLE 3
Combinations of genes targeted by a multiplexed
epigenetic-modifying DNA-targeting system for
increasing transcription of target genes

| first gene | second gene | third gene |
|---|---|---|
| BATF | CD28 | — |
| BATF | EOMES | — |
| BATF | IL-2 | — |
| BATF | IL-2RB | — |
| BATF | IRF4 | — |
| BATF | LAT | — |
| BATF | LCP2 | — |
| BATF | TBX21 | — |
| BATF | VAV1 | — |
| CD28 | EOMES | — |
| CD28 | IL-2 | — |
| CD28 | IL-2RB | — |
| CD28 | IRF4 | — |
| CD28 | LAT | — |
| CD28 | LCP2 | — |
| CD28 | TBX21 | — |
| CD28 | VAV1 | — |
| EOMES | IL-2 | — |
| EOMES | IL-2RB | — |
| EOMES | IRF4 | — |
| EOMES | LAT | — |
| EOMES | LCP2 | — |
| EOMES | TBX21 | — |
| EOMES | VAV1 | — |
| IL-2 | IL-2RB | — |
| IL-2 | IRF4 | — |
| IL-2 | LAT | — |
| IL-2 | LCP2 | — |
| IL-2 | TBX21 | — |
| IL-2 | VAV1 | — |
| IL-2RB | IRF4 | — |
| IL-2RB | LAT | — |
| IL-2RB | LCP2 | — |
| IL-2RB | TBX21 | — |
| IL-2RB | VAV1 | — |
| IRF4 | LAT | — |
| IRF4 | LCP2 | — |
| IRF4 | TBX21 | — |
| IRF4 | VAV1 | — |
| LAT | LCP2 | — |
| LAT | TBX21 | — |
| LAT | VAV1 | — |
| LCP2 | TBX21 | — |
| LCP2 | VAV1 | — |
| TBX21 | VAV1 | — |
| BATF | CD28 | EOMES |
| BATF | CD28 | IL-2 |
| BATF | CD28 | IL-2RB |
| BATF | CD28 | IRF4 |
| BATF | CD28 | LAT |
| BATF | CD28 | LCP2 |
| BATF | CD28 | TBX21 |
| BATF | CD28 | VAV1 |
| BATF | EOMES | IL-2 |
| BATF | EOMES | IL-2RB |
| BATF | EOMES | IRF4 |
| BATF | EOMES | LAT |
| BATF | EOMES | LCP2 |
| BATF | EOMES | TBX21 |
| BATF | EOMES | VAV1 |
| BATF | IL-2 | IL-2RB |
| BATF | IL-2 | IRF4 |
| BATF | IL-2 | LAT |
| BATF | IL-2 | LCP2 |
| BATF | IL-2 | TBX21 |
| BATF | IL-2 | VAV1 |
| BATF | IL-2RB | IRF4 |
| BATF | IL-2RB | LAT |
| BATF | IL-2RB | LCP2 |
| BATF | IL-2RB | TBX21 |
| BATF | IL-2RB | VAV1 |
| BATF | IRF4 | LAT |
| BATF | IRF4 | LCP2 |
| BATF | IRF4 | TBX21 |
| BATF | IRF4 | VAV1 |
| BATF | LAT | LCP2 |
| BATF | LAT | TBX21 |
| BATF | LAT | VAV1 |
| BATF | LCP2 | TBX21 |
| BATF | LCP2 | VAV1 |
| BATF | TBX21 | VAV1 |
| CD28 | EOMES | IL-2 |
| CD28 | EOMES | IL-2RB |
| CD28 | EOMES | IRF4 |
| CD28 | EOMES | LAT |
| CD28 | EOMES | LCP2 |
| CD28 | EOMES | TBX21 |
| CD28 | EOMES | VAV1 |
| CD28 | IL-2 | IL-2RB |
| CD28 | IL-2 | IRF4 |
| CD28 | IL-2 | LAT |
| CD28 | IL-2 | LCP2 |
| CD28 | IL-2 | TBX21 |
| CD28 | IL-2 | VAV1 |
| CD28 | IL-2RB | IRF4 |
| CD28 | IL-2RB | LAT |
| CD28 | IL-2RB | LCP2 |
| CD28 | IL-2RB | TBX21 |
| CD28 | IL-2RB | VAV1 |
| CD28 | IRF4 | LAT |
| CD28 | IRF4 | LCP2 |
| CD28 | IRF4 | TBX21 |
| CD28 | IRF4 | VAV1 |
| CD28 | LAT | LCP2 |
| CD28 | LAT | TBX21 |
| CD28 | LAT | VAV1 |
| CD28 | LCP2 | TBX21 |
| CD28 | LCP2 | VAV1 |
| CD28 | TBX21 | VAV1 |
| EOMES | IL-2 | IL-2RB |
| EOMES | IL-2 | IRF4 |
| EOMES | IL-2 | LAT |
| EOMES | IL-2 | LCP2 |
| EOMES | IL-2 | TBX21 |
| EOMES | IL-2 | VAV1 |
| EOMES | IL-2RB | IRF4 |
| EOMES | IL-2RB | LAT |
| EOMES | IL-2RB | LCP2 |
| EOMES | IL-2RB | TBX21 |
| EOMES | IL-2RB | VAV1 |
| EOMES | IRF4 | LAT |
| EOMES | IRF4 | LCP2 |
| EOMES | IRF4 | TBX21 |
| EOMES | IRF4 | VAV1 |
| EOMES | LAT | LCP2 |
| EOMES | LAT | TBX21 |
| EOMES | LAT | VAV1 |
| EOMES | LCP2 | TBX21 |
| EOMES | LCP2 | VAV1 |
| EOMES | TBX21 | VAV1 |
| IL-2 | IL-2RB | IRF4 |
| IL-2 | IL-2RB | LAT |
| IL-2 | IL-2RB | LCP2 |
| IL-2 | IL-2RB | TBX21 |
| IL-2 | IL-2RB | VAV1 |
| IL-2 | IRF4 | LAT |
| IL-2 | IRF4 | LCP2 |
| IL-2 | IRF4 | TBX21 |
| IL-2 | IRF4 | VAV1 |
| IL-2 | LAT | LCP2 |
| IL-2 | LAT | TBX21 |
| IL-2 | LAT | VAV1 |
| IL-2 | LCP2 | TBX21 |
| IL-2 | LCP2 | VAV1 |
| IL-2 | TBX21 | VAV1 |
| IL-2RB | IRF4 | LAT |
| IL-2RB | IRF4 | LCP2 |
| IL-2RB | IRF4 | TBX21 |
| IL-2RB | IRF4 | VAV1 |
| IL-2RB | LAT | LCP2 |
| IL-2RB | LAT | TBX21 |
| IL-2RB | LAT | VAV1 |
| IL-2RB | LCP2 | TBX21 |
| IL-2RB | LCP2 | VAV1 |
| IL-2RB | TBX21 | VAV1 |
| IRF4 | LAT | LCP2 |
| IRF4 | LAT | TBX21 |
| IRF4 | LAT | VAV1 |
| IRF4 | LCP2 | TBX21 |
| IRF4 | LCP2 | VAV1 |
| IRF4 | TBX21 | VAV1 |
| LAT | LCP2 | TBX21 |
| LAT | LCP2 | VAV1 |
| LAT | TBX21 | VAV1 |
| LCP2 | TBX21 | VAV1 |

In some embodiments, the DNA-targeting system targets a target site for BATF, CD28, EOMES, IL-2, IL2RB, IRF4, LAT, LCP2, TBX21, and/or VAV1. In some embodiments, the target site comprises a sequence selected from any one of SEQ ID NOS:7-9, 78, 144-156, 170, 172-177, and 184-191, or a contiguous portion thereof of at least 14 nucleotides, or a complementary sequence of any of the foregoing. In some embodiments, the target site is a contiguous portion of any one of SEQ ID NOS:7-9, 78, 144-156, 170, 172-177, and 184-191 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS:7-9, 78, 144-156, 170, 172-177, and 184-191. In some embodiments, the target site is a sequence set forth in Table 6.

In some embodiments, the DNA-targeting system targets a target site for IL-2, EOMES, LCP2, and/or TBX21.

In some embodiments, the DNA-targeting system targets a target site for IL-2. In some embodiments, the target site for regulating transcription of IL-2 is within the coordinates chr4:122,456,711-122,456,729. In some embodiments, the target site is within the coordinates chr4: 122,456,611-122,456,829. In some embodiments, the target site is within the coordinates chr4: 122,456,661-122,456,779. In some embodiments, the target site is within the coordinates chr4: 122,456,691-122,456,749. In some embodiments, the target site is or includes the coordinates chr4:122,456,711-122,456,729. In some embodiments the target site for IL-2 is located within 500 bp of human genome assembly GRCh38 (hg38) genomic coordinates chr4: 122,451,470 (e.g., a target site that is +500 of 122,451,470 or −500 of 122,451,470 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr4: 122,451,470. In some embodiments the target site is located within about 80 bp of the genomic coordinate chr4: 122,451,470. In some embodiments, the target site in the region from −40 to +40 of the genomic coordinate 122,451,470. In some embodiments the target site is located within 20 bp of the genomic coordinate chr4: 122,451,470. In some embodiments, the gRNA targets a target site in the region from −10 to +10 of the genomic coordinate chr4: 122,451,470. In some embodiments, any of such target sites include or span the genomic coordinate chr4: 122,451,470, which is a IL-2 transcription start site (TSS). In some embodiments, the target site comprises a sequence selected from SEQ ID NO:78, or a contiguous portion thereof of at least 14 nucleotides, or a complementary sequence of any of the foregoing. In some embodiments, the target site is a contiguous portion of SEQ ID NO:78 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:78.

In some embodiments, the DNA-targeting system targets a target site for EOMES. In some embodiments, the target site for regulating EOMES is within the coordinates chr3:27,722,421-27,722,440. In some embodiments, the target site is within the coordinates chr3: 27,722,321-27,722,540. In some embodiments, the target site is within the coordinates chr3: 27,722,371-27,722,490. In some embodiments, the target site is within the coordinates chr3: 27,722,401-27,722,460. In some embodiments, the target site is or includes the coordinates chr3:27,722,421-27,722,440. In some embodiments the target site for EOMES is located within 500 bp of human genome assembly GRCh38 (hg38) genomic coordinates chr3: 27,715,953 (e.g., a target site that is +500 of 27,715,953 or −500 of 27,715,953 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr3: 50,606,489. In some embodiments the target site is located within about 80 bp of the genomic coordinate chr3: 27,715,953. In some embodiments, the target site in the region from −40 to +40 of the genomic coordinate 27,715,953. In some embodiments the target site is located within 20 bp of the genomic coordinate chr3: 27,715,953. In some embodiments, the gRNA targets a target site in the region from −10 to +10 of the genomic coordinate chr3: 27,715,953. In some embodiments, any of such target sites include or span the genomic coordinate chr3: 27,715,953, which is a EOMES transcription start site (TSS). In some embodiments, the target site comprises a sequence selected from SEQ ID NO:149, or a contiguous portion thereof of at least 14 nucleotides, or a complementary sequence of any of the foregoing. In some embodiments, the target site is a contiguous portion of SEQ ID NO:149 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:149.

In some embodiments, the DNA-targeting system targets a target site for LCP2. In some embodiments, the target site for regulating LCP2 is within the coordinates chr5:170,298,278-170,298,297. In some embodiments, the target site is within the coordinates chr5: 170,298,178-170,298,397. In some embodiments, the target site is within the coordinates chr5: 170,298,228-170,298,347. In some embodiments, the target site is within the coordinates chr5: 170,298,258-170,298,317. In some embodiments, the target site is or includes the coordinates chr5:170,298,278-170,298,297. In some embodiments the target site for LCP2 is located within 500 bp of human genome assembly GRCh38 (hg38) genomic coordinates chr5: 170,246,233 (e.g., a target site that is +500 of 170,246,233 or −500 of 170,246,233 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr5: 170,246,233. In some embodiments the target site is located within about 80 bp of the genomic coordinate chr5: 170,246,233. In some embodiments, the target site in the region from −40 to +40 of the genomic coordinate 170,246,233. In some embodiments the target site is located within 20 bp of the genomic coordinate chr5: 170,246,233. In some embodiments, the gRNA targets a target site in the region from −10 to +10 of the genomic coordinate chr5: 170,246,233. In some embodiments, any of such target sites include or span the genomic coordinate chr5: 170,246,233, which is a LCP2 transcription start site (TSS). In some embodiments, the target site comprises a sequence selected from SEQ ID NO:151, or a contiguous portion thereof of at least 14 nucleotides, or a complementary sequence of any of the foregoing. In some embodiments, the target site is a contiguous portion of SEQ ID NO:151 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:151.

In some embodiments, the DNA-targeting system targets a target site for TBX21. In some embodiments, the target site for regulating TBX21 is within the coordinates chr17:47,733,109-47,733,128. In some embodiments, the target site is within the coordinates chr17: 47,733,009-47,733,228. In some embodiments, the target site is within the coordinates chr17: 47,733,059-47,733,178. In some embodiments, the target site is within the coordinates chr17: 47,733,089-47,733,148. In some embodiments, the target site is or includes the coordinates chr17:47,733,109-47,733,128. In some embodiments the target site for TBX21 is located within 500 bp of human genome assembly GRCh38 (hg38) genomic coordinates chr17: 41,733,236 (e.g., a target site that is +500 of 41,733,236 or −500 of 41,733,236 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr17: 41,733,236. In some embodiments the target site is located within about 80 bp of the genomic coordinate chr17: 41,733,236. In some embodiments, the target site in the region from −40 to +40 of the genomic coordinate 41,733,236. In some embodiments the target site is located within 20 bp of the genomic coordinate chr17: 41,733,236. In some embodiments, the gRNA targets a target site in the region from −10 to +10 of the genomic coordinate chr17: 41,733,236. In some embodiments, any of such target sites include or span the genomic coordinate chr17: 41,733,236, which is a TBX21 transcription start site (TSS). In some embodiments, the target site comprises a sequence selected from SEQ ID NO:155, or a contiguous portion thereof of at least 14 nucleotides, or a complementary sequence of any of the foregoing. In some embodiments, the target site is a contiguous portion of SEQ ID NO:155 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in SEQ ID NO:155.

In some embodiments, the DNA-targeting system targets a combination of genes, such as any combination shown in Table 1. In some embodiments, the DNA-targeting system targets a combination of genes selected from: BATF and IL-2; BATF and VAV1; CD28 and BATF; CD28 and EOMES; CD28 and IL-2; CD28 and LCP2; CD28 and TBX21; CD28 and VAV1; EOMES and BATF; EOMES and LCP2; EOMES and TBX21; EOMES and VAV1; LCP2 and BATF; LCP2 and IL-2; LCP2 and TBX21; LCP2 and VAV1; TBX21 and BATF; TBX21 and IL-2; TBX21 and TBX21; TBX21 and VAV1; and VAV1 and IL-2.

In some embodiments, the DNA-targeting system targets IL-2 and VAV1. n some embodiments, the target site for targeting IL-2 can be any as described above, and the target site for targeting VAV1 can be any as described above. In some embodiments, the DNA-targeting system targets a target site for IL-2 comprising the sequence set forth in SEQ ID NO:78, and a target site for VAV1 comprising the sequence set forth in SEQ ID NO:170.

In some embodiments, the DNA-targeting system targets IL-2 and LCP2. In some embodiments, the target site for targeting IL-2 can be any as described above, and the target site for targeting LCP2 can be any as described above. In some embodiments, the DNA-targeting system targets a target site for IL-2 comprising the sequence set forth in SEQ ID NO:78, and a target site for LCP2 comprising the sequence set forth in SEQ ID NO:151.

In some embodiments, the DNA-targeting system targets IL-2 and TBX21. In some embodiments, the target site for targeting IL-2 can be any as described above, and the target site for targeting TBX21 can be any as described above. In some embodiments, the DNA-targeting system targets a target site for IL-2 comprising the sequence set forth in SEQ ID NO:78, and a target site for TBX21 comprising the sequence set forth in SEQ ID NO:155.

In some embodiments, the DNA-targeting system targets IL-2 and EOMES. In some embodiments, the target site for targeting IL-2 can be any as described above, and the target site for targeting EOMES can be any as described above. In some embodiments, the DNA-targeting system targets a target site for IL-2 comprising the sequence set forth in SEQ ID NO:78, and a target site for EOMES comprising the sequence set forth in SEQ ID NO:149.

In some embodiments, the DNA-targeting system targets IL2RB and VAV1.

In some embodiments, the DNA-targeting system targets a combination of target sites for a combination of genes for transcriptional activation, as shown in Table 4.

TABLE 4
Gene and target site combinations for transcriptional activation

| | Target Site 1 | | Target Site 2 |
|---|---|---|---|
| Target Gene 1 | SEQ ID NO: | Target Gene 2 | SEQ ID NO: |
| BATF | 172 | IL-2 | 78 |
| BATF | 172 | VAV1 | 170 |
| CD28 | 144 | BATF | 172 |
| CD28 | 144 | EOMES | 149 |
| CD28 | 144 | IL-2 | 78 |
| CD28 | 144 | LCP2 | 151 |
| CD28 | 144 | TBX21 | 155 |
| CD28 | 144 | VAV1 | 170 |
| EOMES | 149 | BATF | 172 |
| EOMES | 149 | LCP2 | 151 |
| EOMES | 149 | TBX21 | 155 |
| EOMES | 149 | VAV1 | 170 |
| LCP2 | 151 | BATF | 172 |
| LCP2 | 151 | IL-2 | 78 |
| LCP2 | 151 | TBX21 | 155 |
| LCP2 | 151 | VAV1 | 170 |
| TBX21 | 155 | BATF | 172 |
| TBX21 | 155 | IL-2 | 78 |
| TBX21 | 155 | TBX21 | 155 |
| TBX21 | 155 | VAV1 | 170 |
| VAV1 | 170 | IL-2 | 78 |

In some embodiments, the DNA-targeting system targets a target site for BATF comprising the sequence set forth in SEQ ID NO:172, and a target site for IL-2 comprising the sequence set forth in SEQ ID NO:78. In some embodiments, the DNA-targeting system targets a target site for BATF comprising the sequence set forth in SEQ ID NO:172, and a target site for VAV1 comprising the sequence set forth in SEQ ID NO:170. In some embodiments, the DNA-targeting system targets a target site for CD28 comprising the sequence set forth in SEQ ID NO:144, and a target site for BATF comprising the sequence set forth in SEQ ID NO:172. In some embodiments, the DNA-targeting system targets a target site for CD28 comprising the sequence set forth in SEQ ID NO:144, and a target site for EOMES comprising the sequence set forth in SEQ ID NO:149. In some embodiments, the DNA-targeting system targets a target site for CD28 comprising the sequence set forth in SEQ ID NO:144, and a target site for IL-2 comprising the sequence set forth in SEQ ID NO:78. In some embodiments, the DNA-targeting system targets a target site for CD28 comprising the sequence set forth in SEQ ID NO:144, and a target site for LCP2 comprising the sequence set forth in SEQ ID NO:151. In some embodiments, the DNA-targeting system targets a target site for CD28 comprising the sequence set forth in SEQ ID NO:144, and a target site for TBX21 comprising the sequence set forth in SEQ ID NO:155. In some embodiments, the DNA-targeting system targets a target site for CD28 comprising the sequence set forth in SEQ ID NO:144, and a target site for VAV1 comprising the sequence set forth in SEQ ID NO:170. In some embodiments, the DNA-targeting system targets a target site for EOMES comprising the sequence set forth in SEQ ID NO:149, and a target site for BATF comprising the sequence set forth in SEQ ID NO:172. In some embodiments, the DNA-targeting system targets a target site for EOMES comprising the sequence set forth in SEQ ID NO:149, and a target site for LCP2 comprising the sequence set forth in SEQ ID NO:151. In some embodiments, the DNA-targeting system targets a target site for EOMES comprising the sequence set forth in SEQ ID NO:149, and a target site for TBX21 comprising the sequence set forth in SEQ ID NO:155. In some embodiments, the DNA-targeting system targets a target site for EOMES comprising the sequence set forth in SEQ ID NO:149, and a target site for VAV1 comprising the sequence set forth in SEQ ID NO:170. In some embodiments, the DNA-targeting system targets a target site for LCP2 comprising the sequence set forth in SEQ ID NO:151, and a target site for BATF comprising the sequence set forth in SEQ ID NO:172. In some embodiments, the DNA-targeting system targets a target site for LCP2 comprising the sequence set forth in SEQ ID NO:151, and a target site for IL-2 comprising the sequence set forth in SEQ ID NO:78. In some embodiments, the DNA-targeting system targets a target site for LCP2 comprising the sequence set forth in SEQ ID NO:151, and a target site for TBX21 comprising the sequence set forth in SEQ ID NO:155. In some embodiments, the DNA-targeting system targets a target site for LCP2 comprising the sequence set forth in SEQ ID NO:151, and a target site for VAV1 comprising the sequence set forth in SEQ ID NO:170. In some embodiments, the DNA-targeting system targets a target site for TBX21 comprising the sequence set forth in SEQ ID NO:155, and a target site for BATF comprising the sequence set forth in SEQ ID NO:172. In some embodiments, the DNA-targeting system targets a target site for TBX21 comprising the sequence set forth in SEQ ID NO:155, and a target site for IL-2 comprising the sequence set forth in SEQ ID NO:78. In some embodiments, the DNA-targeting system targets a target site for TBX21 comprising the sequence set forth in SEQ ID NO:155, and a target site for TBX21 comprising the sequence set forth in SEQ ID NO:155. In some embodiments, the DNA-targeting system targets a target site for TBX21 comprising the sequence set forth in SEQ ID NO:155, and a target site for VAV1 comprising the sequence set forth in SEQ ID NO:170. In some embodiments, the DNA-targeting system targets a target site for VAV1 comprising the sequence set forth in SEQ ID NO:170, and a target site for IL-2 comprising the sequence set forth in SEQ ID NO:78.

In some embodiments, delivery of the DNA-targeting system increases expression (e.g. transcription) of one or more genes. In some embodiments, the increase in gene expression in a cell (e.g. T cell) is about a log 2 fold change of greater than 1.0. For instance, the log 2 fold change is greater than at or about 1.5, at or about 2.0, at or about 2.5, at or about 3.0, at or about 4.0, at or about 5.0, at or about 6.0, at or about 7.0, at or about 8.0, at or about 9.0, at or about 10.0 or any value between any of the foregoing compared to the level of the gene in a control cell.

### C. CRISPR/Cas-Based DNA-Targeting Systems and DNA-Binding Domains

Provided herein are multiplexed epigenetic-targeting DNA-targeting systems based on CRISPR/Cas systems, i.e. CRISPR/Cas-based DNA-targeting systems, that are able to bind to a target site for a target gene, or to a combination of target sites, e.g. for a combination of target genes. In some embodiments, the CRISPR/Cas DNA-binding domain is nuclease inactive, such as includes a dCas (e.g. dCas9) so that the system binds to the target site for a target gene without mediating nucleic acid cleavage at the target site. The CRISPR/Cas-based DNA-targeting systems may be used to modulate expression of a target gene in a cell, such as a T cell. In some embodiments, the target gene may include any as described herein, including any described above in Section I.B. In some embodiments, the target site for the target gene may include any as described herein, including any described above in Section I.B. In some embodiments, the CRISPR/Cas-based DNA-targeting system can include any known Cas enzyme, and generally a nuclease-inactive or dCas. In some embodiments, the CRISPR/Cas-based DNA-targeting system includes a fusion protein of a nuclease-inactive Cas protein or a variant thereof and an effector domain, and at least one gRNA. In some embodiments, the effector domain reduces transcription of the one or more genes (e.g. the effector domain is a transcriptional repressor, such as any described in Section I.E.1). In some embodiments, the effector domain increases transcription of the one or more genes (e.g. the effector domain is a transcriptional activator, such as any described in Section I.E.2).

The CRISPR system (also known as CRISPR/Cas system, or CRISPR-Cas system) refers to a conserved microbial nuclease system, found in the genomes of bacteria and archaea, that provides a form of acquired immunity against invading phages and plasmids. Clustered Regularly Interspaced Short Palindromic Repeats (CRISPR), refers to loci containing multiple repeating DNA elements that are separated by non-repeating DNA sequences called spacers. Spacers are short sequences of foreign DNA that are incorporated into the genome between CRISPR repeats, serving as a “memory” of past exposures. Spacers encode the DNA-targeting portion of RNA molecules that confer specificity for nucleic acid cleavage by the CRISPR system. CRISPR loci contain or are adjacent to one or more CRISPR-associated (Cas) genes, which can act as RNA-guided nucleases for mediating the cleavage, as well as non-protein coding DNA elements that encode RNA molecules capable of programming the specificity of the CRISPR-mediated nucleic acid cleavage.

In Type II CRISPR/Cas systems with the Cas protein Cas9, two RNA molecules and the Cas9 protein form a ribonucleoprotein (RNP) complex to direct Cas9 nuclease activity. The CRISPR RNA (crRNA) contains a spacer sequence that is complementary to a target nucleic acid sequence (target site), and that encodes the sequence specificity of the complex. The trans-activating crRNA (tracrRNA) base-pairs to a portion of the crRNA and forms a structure that complexes with the Cas9 protein, forming a Cas/RNA RNP complex.

Naturally occurring CRISPR/Cas systems, such as those with Cas9, have been engineered to allow efficient programming of Cas/RNA RNPs to target desired sequences in cells of interest, both for gene-editing and modulation of gene expression. The tracrRNA and crRNA have been engineered to form a single chimeric guide RNA molecule, commonly referred to as a guide RNA (gRNA), for example as described in WO 2013/176772, WO 2014/093661, WO 2014/093655, Jinek, M. et al. Science 337(6096):816-21 (2012), or Cong, L. et al. Science 339(6121):819-23 (2013). The spacer sequence of the gRNA can be chosen by a user to target the Cas/gRNA RNP complex to a desired locus, e.g. a desired target site in the target gene.

Cas proteins have also been engineered to be catalytically inactivated or nuclease inactive to allow targeting of Cas/gRNA RNPs without inducing cleavage at the target site. Mutations in Cas proteins can reduce or abolish nuclease activity of the Cas protein, rendering the Cas protein catalytically inactive. Cas proteins with reduced or abolished nuclease activity are referred to as deactivated Cas (dCas), or nuclease-inactive Cas (iCas) proteins, as referred to interchangeably herein. An exemplary deactivated Cas9 (dCas9) derived from S. pyogenes contains silencing mutations of the RuvC and HNH nuclease domains (D10A and H840A), for example as described in WO 2013/176772, WO 2014/093661, Jinek, M. et al. Science 337(6096):816-21 (2012), and Qi, L. et al. Cell 152(5):1173-83 (2013). Exemplary dCas variants derived from the Cas12 system (i.e. Cpf1) are described, for example in WO 2017/189308 and Zetsche, B. et al. Cell 163(3):759-71 (2015). Conserved domains that mediate nucleic acid cleavage, such as RuvC and HNH endonuclease domains, are readily identifiable in Cas orthologues, and can be mutated to produce inactive variants, for example as described in Zetsche, B. et al. Cell 163(3):759-71 (2015).

dCas-fusion proteins with transcriptional and/or epigenetic regulators have been used as a versatile platform for ectopically regulating gene expression in target cells. These include fusion of a Cas with an effector domain, such as a transcriptional activator or transcriptional repressor. For example, fusing dCas9 with a transcriptional activator such as VP64 (a polypeptide composed of four tandem copies of VP16, a 16 amino acid transactivation domain of the Herpes simplex virus) can result in robust induction of gene expression. Alternatively, fusing dCas9 with a transcriptional repressor such as KRAB (Kruppel associated box) can result in robust repression of gene expression. A variety of dCas-fusion proteins with effector domains can be engineered for regulation of gene expression, for example as described in WO 2014/197748, WO 2016/130600, WO 2017/180915, WO 2021/226555, WO 2013/176772, WO 2014/152432, WO 2014/093661, WO 2021/247570, Adli, M. Nat. Commun. 9, 1911 (2018), Perez-Pinera, P. et al. Nat. Methods 10, 973-976 (2013), Mali, P. et al. Nat. Biotechnol. 31, 833-838 (2013), Maeder, M. L. et al. Nat. Methods 10, 977-979 (2013), Gilbert, L. A. et al. Cell 154(2):442-451 (2013), and Nunez, J. K. et al. Cell 184(9):2503-2519 (2021).

In some aspects, provided is a DNA-targeting system comprising a fusion protein comprising a DNA-binding domain comprising a nuclease-inactive Cas protein or variant thereof, and at least one effector domain for reducing transcription or inducing transcriptional repression (i.e. a transcriptional repressor) when targeted to a target gene in a cell (e.g. a T cell). In some embodiments, the dCas protein is any suitable dCas protein, such as any described in section I.C. In some embodiments, the dCas protein is a dCas9 protein, such dSpCas9 or dSaCas9. In some embodiments, the at least one effector domain is any suitable transcriptional repressor effector domain, such as any described in Section I.E.1, such as KRAB and/or DNMT3A/L. In some embodiments, the at least one effector domain is KRAB. In some embodiments, the fusion protein is a dCas9-KRAB or dCas9-KRAB-DNMT3A/L fusion protein, for example as described in Section I.F. In such embodiments, the DNA-targeting system also includes one or more gRNAs (e.g. as described in Section I.C.2.a), provided in combination or as a complex with the dCas protein or variant thereof, for targeting of the DNA-targeting system to the target site of the target gene. In some embodiments, the fusion protein is guided to a specific target site sequence of the target gene by the guide RNA, wherein the effector domain mediates targeted epigenetic modification to reduce or repress transcription of the target gene. In some embodiments, a combination of gRNAs guides the fusion protein to a combination of target site sequences in a combination of genes, wherein the effector domain mediates targeted epigenetic modification to reduce or repress transcription of the combination of target genes. Any of a variety of effector domains that reduce or repress transcription can be used as described further below.

In some aspects, provided is a DNA-targeting system comprising a fusion protein comprising a DNA-binding domain comprising a nuclease-inactive Cas protein or variant thereof, and an effector domain for increasing transcription or inducing transcriptional activation (i.e. a transcriptional activator) when targeted to a target gene in a cell (e.g. a T cell). In some embodiments, the dCas protein is any suitable dCas protein, such as any described in section I.C. In some embodiments, the dCas protein is a dCas9 protein, such dSpCas9 or dSaCas9. In some embodiments, the at least one effector domain is any suitable transcriptional activator effector domain, such as any described in Section I.E..2, such as VP64. In some embodiments, the at least one effector domain is VP64. In some embodiments, the fusion protein is a dCas9-VP64 fusion protein, for example as described in Section I.F. In such embodiments, the DNA-targeting system also includes one or more gRNAs (e.g. as described in Section I.C.2.b), provided in combination or as a complex with the dCas protein or variant thereof, for targeting of the DNA-targeting system to the target site of the target gene. In some embodiments, the fusion protein is guided to a specific target site sequence of the target gene by the guide RNA, wherein the effector domain mediates targeted epigenetic modification to increase or activate transcription of the target gene. In some embodiments, a combination of gRNAs guides the fusion protein to a combination of target site sequences in a combination of genes, wherein the effector domain mediates targeted epigenetic modification to increase or activate transcription of the combination of target genes. Any of a variety of effector domains that increase or activate transcription can be used as described further below.

### 1. CRISPR/Cas-Based DNA-Biding Domains

In some aspects, the DNA-binding domain comprises a CRISPR-associated (Cas) protein or variant thereof, or is derived from a Cas protein or variant thereof. In particular embodiments here, the Cas protein is nuclease-inactive (i.e. is a dCas protein).

In some embodiments, the Cas protein is derived from a Class 1 CRISPR system (i.e. multiple Cas protein system), such as a Type I, Type III, or Type IV CRISPR system. In some embodiments, the Cas protein is derived from a Class 2 CRISPR system (i.e. single Cas protein system), such as a Type II, Type V, or Type VI CRISPR system. In some embodiments, the Cas protein is from a Type V CRISPR system. In some embodiments, the Cas protein is derived from a Cas12 protein (i.e. Cpf1) or variant thereof, for example as described in WO 2017/189308 and Zetsche, B. et al. Cell. 163(3):759-71 (2015). In some embodiments, the Cas protein is derived from a Type II CRISPR system. In some embodiments, the Cas protein is derived from a Cas9 protein or variant thereof, for example as described in WO 2013/176772, WO 2014/152432, WO 2014/093661, WO 2014/093655, Jinek, M. et al. Science 337(6096):816-21 (2012), Mali, P. et al. Science 339(6121):823-6 (2013), Cong, L. et al. Science 339(6121):819-23 (2013), Perez-Pinera, P. et al. Nat. Methods 10, 973-976 (2013), or Mali, P. et al. Nat. Biotechnol. 31, 833-838 (2013). Various CRISPR/Cas systems and associated Cas proteins for use in gene editing and regulation have been described, for example in Moon, S. B. et al. Exp. Mol. Med. 51, 1-11 (2019), Zhang, F. Q. Rev. Biophys. 52, E6 (2019), and Makarova K.S. et al. Methods Mol. Biol. 1311:47-75 (2015).

In some embodiments, the dCas9 protein can comprise a sequence derived from a naturally occurring Cas9 molecule, or variant thereof. In some embodiments, the dCas9 protein can comprise a sequence derived from a naturally occurring Cas9 molecule of S. pyogenes, S. thermophilus, S. aureus, C. jejuni, N. meningitidis, F. novicida, S. canis, S. auricularis, or variant thereof. In some embodiments, the dCas9 protein comprises a sequence derived from a naturally occurring Cas9 molecule of S. aureus. In some embodiments, the dCas9 protein comprises a sequence derived from a naturally occurring Cas9 molecule of S. pyogenes.

Non-limiting examples of Cas9 orthologs from other bacterial strains include but are not limited to: Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1147; Caldicelulosiruptor becscii DSM 6725; Candidatus Desulforudis audaxviator MP104C; Caldicellulosiruptor hydrothermalis 108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

In some aspects, the Cas protein is a variant that lacks nuclease activity (i.e. is a dCas protein). In some embodiments, the Cas protein is mutated so that nuclease activity is reduced or eliminated. Such Cas proteins are referred to as deactivated Cas or dead Cas (dCas) or nuclease-inactive Cas (iCas) proteins, as referred to interchangeably herein. In some embodiments, the variant Cas protein is a variant Cas9 protein that lacks nuclease activity or that is a deactivated Cas9 (dCas9, or iCas9) protein.

In some embodiments, the Cas9 protein or a variant thereof is derived from a Staphylococcus aureus Cas9 (SaCas9) protein or a variant thereof. In some embodiments, the variant Cas9 is a Staphylococcus aureus dCas9 protein (dSaCas9) that comprises at least one amino acid mutation selected from D10A and N580A, with reference to numbering of positions of SEQ ID NO:124. In some embodiments, the variant Cas9 protein comprises the sequence set forth in SEQ ID NO:125, or an amino acid sequence that has at least 90%, 91%, 92%, 93%, 94%, 95%, 96%, 97%, 98%, or 99% sequence identity thereto.

In some embodiments, the Cas9 protein or variant thereof is derived from a Streptococcus pyogenes Cas9 (SpCas9) protein or a variant thereof. In some embodiments, the variant Cas9 is a Streptococcus pyogenes dCas9 (dSpCas9) protein that comprises at least one amino acid mutation selected from D10A and H840A, with reference to numbering of positions of SEQ ID NO:126. In some embodiments, the variant Cas9 protein comprises the sequence set forth in SEQ ID NO:127, or an amino acid sequence that has at least 90%, 91%, 92%, 93%, 94%, 95%, 96%, 97%, 98%, or 99% sequence identity thereto.

2 Guide RNAs (gRNAs)

In some embodiments, the Cas protein (e.g. dCas9) is provided in combination or as a complex with one or more guide RNA (gRNA). In some aspects, the gRNA is a nucleic acid that promotes the specific targeting or homing of the gRNA/Cas RNP complex to the target site of the target gene, such as any described above in Section I.B. In some embodiments, a target site of a gRNA may be referred to as a protospacer.

Provided herein are gRNAs, such as gRNAs that target or bind to a target site for a gene, such as in a target gene or regulatory DNA element thereof, such as any described herein, for example in Section I.B. In some embodiments, the gRNA is capable of complexing with the Cas protein or variant thereof. In some embodiments, the gRNA comprises a gRNA spacer sequence (i.e. a spacer sequence or a guide sequence) that is capable of hybridizing to the target site, or that is complementary to the target site, such as any target site described herein. In some embodiments, the gRNA comprises a scaffold sequence that complexes with or binds to the Cas protein.

In some embodiments, the gRNAs provided herein are chimeric gRNAs. In general, gRNAs can be unimolecular (i.e. composed of a single RNA molecule), or modular (comprising more than one, and typically two, separate RNA molecules). Modular gRNAs can be engineered to be unimolecular, wherein sequences from the separate modular RNA molecules are comprised in a single gRNA molecule, sometimes referred to as a chimeric gRNA, synthetic gRNA, or single gRNA. In some embodiments, the chimeric gRNA is a fusion of two non-coding RNA sequences: a crRNA sequence and a tracrRNA sequence, for example as described in WO 2013/176772, or Jinek, M. et al. Science 337(6096):816-21 (2012). In some embodiments, the chimeric gRNA mimics the naturally occurring crRNA:tracrRNA duplex involved in the Type II Effector system, wherein the naturally occurring crRNA:tracrRNA duplex acts as a guide for the Cas9 protein.

In some aspects, the spacer sequence of a gRNA is a polynucleotide sequence comprising at least a portion that has sufficient complementarity with the target site to hybridize with the target site in the target gene and direct sequence-specific binding of a Cas/gRNA complex to the sequence of the target site. Full complementarity is not necessarily required, provided there is sufficient complementarity to cause hybridization. In some embodiments, the gRNA comprises a spacer sequence that is complementary, e.g., at least 80%, 85%, 90%, 95%, 98%, 99%, or 100% (e.g., fully complementary), to the target site. The strand of the target nucleic acid comprising the target site sequence may be referred to as the “complementary strand” of the target nucleic acid.

In some aspects, a gRNA targets a target site in double-stranded DNA. Thus, in some aspects, the sequence of the target site may be defined by the sequence that the gRNA spacer hybridizes to, or by the sequence complementary to the sequence that the gRNA spacer hybridizes to. In some aspects, the sequence of the target site may be defined by the sequence that the gRNA spacer displaces in order to hybridize to the DNA. In some embodiments, the sequence of the target site is the sequence that the gRNA hybridizes to.

In some embodiments, the gRNA spacer sequence is between about 14 nucleotides (nt) and about 26 nt, or between 16 nt and 22 nt in length. In some embodiments, the gRNA spacer sequence is 14 nt, 15 nt, 16 nt, 17 nt, 18 nt, 19 nt, 20 nt, 21 nt or 22 nt, 23 nt, 24 nt, 25 nt, or 26 nt in length. In some embodiments, the gRNA spacer sequence is 18 nt, 19 nt, 20 nt, 21 nt or 22 nt in length. In some embodiments, the gRNA spacer sequence is 20 nt in length.

A target site of a gRNA may be referred to as a protospacer. In some aspects, the spacer is designed to target a protospacer with a specific protospacer-adjacent motif (PAM), i.e. a sequence immediately adjacent to the protospacer that contributes to and/or is required for Cas binding specificity. Different CRISPR/Cas systems have different PAM requirements for targeting. For example, in some embodiments, S. pyogenes Cas9 uses the PAM 5′-NGG-3′ (SEQ ID NO: 224), where N is any nucleotide. In some embodiments, S. aureus Cas9 uses the PAM 5′-NNGRRT-3′ (SEQ ID NO: 225), where N is any nucleotide, and R is G or A. In some embodiments, N. meningitidis Cas9 uses the PAM 5′-NNNNGATT-3′ (SEQ ID NO: 226), where N is any nucleotide. In some embodiments, C. jejuni Cas9 uses the PAM 5′-NNNNRYAC-3′ (SEQ ID NO: 227), where N is any nucleotide, R is G or A, and Y is C or T. In some embodiments, S. thermophilus uses the PAM 5′-NNAGAAW-3′ (SEQ ID NO: 228), where N is any nucleotide and W is A or T. In some embodiments, F. novicida Cas9 uses the PAM 5′-NGG-3′ (SEQ ID NO: 224), where N is any nucleotide. In some embodiments, T. denticola Cas9 uses the PAM 5′-NAAAAC-3′ (SEQ ID NO: 229), where N is any nucleotide. In some embodiments, Cas12a (also known as Cpf1) from various species, uses the PAM 5′-TTTV-3′ (SEQ ID NO: 230). In some embodiments, Cas proteins may use or be engineered to use different PAMs from those listed above. For example, mutated SpCas9 proteins may use the PAMs 5′-NGG-3′ (SEQ ID NO: 224), 5′-NGAN-3′ (SEQ ID NO: 231), 5′-NGNG-3′ (SEQ ID NO: 232), 5′-NGAG-3′ (SEQ ID NO: 233), or 5′-NGCG-3′ (SEQ ID NO: 234). In some embodiments, the protospacer-adjacent motif (PAM) of a gRNA for complexing with S. pyogenes Cas9 or variant thereof is NGG, as set forth in SEQ ID NO: 224. In some embodiments, the PAM of a gRNA for complexing with S. aureus Cas9 or variant thereof is NNGRRT, as set forth in SEQ ID NO: 225.

A spacer sequence may be selected to reduce the degree of secondary structure within the spacer sequence. Secondary structure may be determined by any suitable polynucleotide folding algorithm.

In some embodiments, the gRNA (including the guide sequence) will comprise the base uracil (U), whereas DNA encoding the gRNA molecule will comprise the base thymine (T). While not wishing to be bound by theory, in some embodiments, it is believed that the complementarity of the guide sequence with the target sequence contributes to specificity of the interaction of the gRNA molecule/Cas molecule complex with a target nucleic acid. It is understood that in a guide sequence and target sequence pair, the uracil bases in the guide sequence will pair with the adenine bases in the target sequence.

In some embodiments, one, more than one, or all of the nucleotides of a gRNA can have a modification, e.g., to render the gRNA less susceptible to degradation and/or improve bio-compatibility. By way of non-limiting example, the backbone of the gRNA can be modified with a phosphorothioate, or other modification(s). In some cases, a nucleotide of the gRNA can comprise a 2′ modification, e.g., a 2-acetylation, e.g., a 2′ methylation, or other modification(s).

Methods for designing gRNAs and exemplary targeting domains can include those described in, e.g., International PCT Pub. Nos. WO 2014/197748, WO 2016/130600, WO 2017/180915, WO 2021/226555, WO 2013/176772, WO 2014/152432, WO 2014/093661, WO 2014/093655, WO 2015/089427, WO 2016/049258, WO 2016/123578, WO 2021/076744, WO 2014/191128, WO 2015/161276, WO 2017/193107, and WO 2017/093969.

a. gRNAs for Transcriptional Repression

In some embodiments, a gRNA provided herein targets a target site for a gene for transcriptional repression, such as any targets site or target gene described in Section I.B.2. In some embodiments, a gRNA provided herein targets a target site for a gene, such as a gene in a T cell, wherein the gene is selected from the list consisting of: CBLB, CCNC, CD5, CISH, DGKZ, ELOB, FAS, Fli1, GATA3, KDM1A, MED12, MYB, PRDM1, TGFBR2, and RASA2. In some embodiments, the gRNA targets the gene for transcriptional repression.

In some embodiments, the gRNA targets a target site that comprises a sequence selected from any one of SEQ ID NOS:1-6, 10-33, 80-90, 102-112, 200-211, 292-295, 300-302, and 306-308, as shown in Table 5, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NOS: 1-6, 10-33, 80-90, 102-112, and 200-211 that is 14, 15, 16, 17, 18 or 19 nucleotides in length. In some embodiments, the target site is set forth in any one of SEQ ID NOS: 1-6, 10-33, 80-90, 102-112, 200-211, 292-295, 300-302, and 306-308.

In some embodiments, the gRNA comprises a spacer sequence selected from any one of SEQ ID NOS:35-40, 44-67, 91-101, 113-123, 212-223, 296-299, 303-305, and 309-311, as shown in Table 5, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:35-40, 44-67, 91-101, 113-123, 212-223, 296-299, 303-305, and 309-311 that is 14, 15, 16, 17, 18 or 19 nucleotides in length. In some embodiments, the spacer sequence of the gRNA is set forth in any one of SEQ ID NOS:35-40, 44-67, 91-101, 113-123, 212-223, 296-299, 303-305, and 309-311.

In some embodiments, the gRNA targets a target site of CBLB. In some embodiments, the gRNA targets a target site that comprises SEQ ID NO:11, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of SEQ ID NO: 11 that is 14, 15, 16, 17, 18 or 19 nucleotides in length. In some embodiments, the target site is set forth in SEQ ID NO: 11. In some embodiments, the gRNA comprises a spacer sequence comprising SEQ ID NO:45, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 45 that is 14, 15, 16, 17, 18 or 19 nucleotides in length. In some embodiments, the spacer sequence of the gRNA is set forth in SEQ ID NO:45.

In some embodiments the gRNA targets a target site of MYB. In some embodiments, the gRNA targets a target site that comprises SEQ ID NO:18, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of SEQ ID NO:18 that is 14, 15, 16, 17, 18 or 19 nucleotides in length. In some embodiments, the target site is set forth in SEQ ID NO: 18. In some embodiments, the gRNA comprises a spacer sequence comprising SEQ ID NO:52, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 52 that is 14, 15, 16, 17, 18 or 19 nucleotides in length. In some embodiments, the spacer sequence of the gRNA is set forth in SEQ ID NO:52.

In some embodiments, the gRNA targets a target site of RASA2. In some embodiments, the gRNA targets a target site that comprises SEQ ID NO:19, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of SEQ ID NO:19 that is 14, 15, 16, 17, 18 or 19 nucleotides in length. In some embodiments, the target site is set forth in SEQ ID NO: 19. In some embodiments, the gRNA comprises a spacer sequence comprising SEQ ID NO:53, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 53 that is 14, 15, 16, 17, 18 or 19 nucleotides in length. In some embodiments, the spacer sequence of the gRNA is set forth in SEQ ID NO:53.

In some embodiments, the gRNA targets a target site of CISH. In some embodiments, the gRNA targets a target site that comprises SEQ ID NO:28, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of SEQ ID NO:28 that is 14, 15, 16, 17, 18 or 19 nucleotides in length. In some embodiments, the target site is set forth in SEQ ID NO: 28. In some embodiments, the gRNA comprises a spacer sequence comprising SEQ ID NO:62, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 62 that is 14, 15, 16, 17, 18 or 19 nucleotides in length. In some embodiments, the spacer sequence of the gRNA is set forth in SEQ ID NO:62.

In some embodiments, the gRNA targets a target site of PRDM1. In some embodiments, the gRNA targets a target site that comprises SEQ ID NO:33, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of SEQ ID NO:33 that is 14, 15, 16, 17, 18 or 19 nucleotides in length. In some embodiments, the target site is set forth in SEQ ID NO:33. In some embodiments, the gRNA comprises a spacer sequence comprising SEQ ID NO:67, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 67 that is 14, 15, 16, 17, 18 or 19 nucleotides in length. In some embodiments, the spacer sequence of the gRNA is set forth in SEQ ID NO:67.

In some embodiments, the gRNA targets a target site of MED12. In some embodiments, the gRNA targets a target site that comprises SEQ ID NO:81, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of SEQ ID NO:81 that is 14, 15, 16, 17, 18 or 19 nucleotides in length. In some embodiments, the target site is set forth in SEQ ID NO: 81. In some embodiments, the gRNA comprises a spacer sequence comprising SEQ ID NO:92, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 92 that is 14, 15, 16, 17, 18 or 19 nucleotides in length. In some embodiments, the spacer sequence of the gRNA is set forth in SEQ ID NO:92.

In some embodiments, the gRNA further comprises a scaffold sequence. In some embodiments, the scaffold sequence comprises the sequence set forth in SEQ ID NO:69 (GUUUAAGAGCUAUGCUGGAAACAGCAUAGCAAGUUUAAAUAAGGCUAGUCCGUUAUCA ACUUGAAAAAGUGGCACCGAGUCGGUGC), or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the scaffold sequence is set forth in SEQ ID NO: 69.

In some embodiments, any of the provided gRNA sequences is complexed with or is provided in combination with a fusion protein comprising Cas9. In some embodiments, the Cas9 is a dCas9. In some embodiments, the dCas9 is a dSpCas9, such as a dSpCas9 set forth in SEQ ID NO:127.

In some embodiments, provided herein is a combination of gRNAs that each target a target site for a gene for transcriptional repression. In some embodiments, provided herein is a multiplexed epigenetic-modifying DNA-targeting system comprising the combination of gRNAs.

In some embodiments, the combination of gRNAs comprises at least two gRNAs targeting at least two different genes for transcriptional repression. In some embodiments, the gRNAs target a combination of genes selected from the combinations of genes listed in Table 1. In some embodiments, each gRNA of the combination of gRNAs is selected from any of the gRNAs described herein for targeted transcriptional repression.

In some embodiments, the combination of gRNAs comprises a first gRNA targeted to a first gene and a second gRNA targeted to a second gene. In some embodiments, the first gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2, the second gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2, and the first and second gRNAs target different genes.

In some embodiments, the first gRNA targets CBLB, and the second gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2. In some embodiments, the first gRNA targets CCNC, and the second gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2. In some embodiments, the first gRNA targets MED12, and the second gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2. In some embodiments, the first gRNA targets MYB, and the second gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2.

In some embodiments, the first gRNA targets CBLB and the second gRNA targets MYB. In some embodiments, the gRNA targeting CBLB can be any as described, and the gRNA targeting MYB can be any as described.In some embodiments, the first gRNA targets a target site for CBLB having the sequence set forth in SEQ ID NO:11 and the second gRNA targets a target site for MYB having the sequence set forth in SEQ ID NO:18.

In some embodiments, the first gRNA targets CBLB and the second gRNA targets CCNC. In some embodiments, the gRNA targeting CBLB can be any as described, and the gRNA targeting CCNC can be any as described. In some embodiments, the first gRNA targets a target site for CBLB having the sequence set forth in SEQ ID NO:11 and the second gRNA targets a target site for CCNC having the sequence set forth in SEQ ID NO:104.

In some embodiments, the first gRNA targets CBLB and the second gRNA targets MED12. In some embodiments, the gRNA targeting CBLB can be any as described, and the gRNA targeting MED12 can be any as described. In some embodiments, the first gRNA targets a target site for CBLB having the sequence set forth in SEQ ID NO:11 and the second gRNA targets a target site for MED12 having the sequence set forth in SEQ ID NO:81.

In some embodiments, the first gRNA targets CBLB and the second gRNA targets RASA2. In some embodiments, the gRNA targeting CBLB can be any as described, and the gRNA targeting RASA2 can be any as described. In some embodiments, the first gRNA targets a target site for CBLB having the sequence set forth in SEQ ID NO:11 and the second gRNA targets a target site for RASA2 having the sequence set forth in SEQ ID NO:19.

In some embodiments, the first gRNA targets CISH and the second gRNA targets MED12. In some embodiments, the gRNA targeting CISH can be any as described, and the gRNA targeting MED12 can be any as described. In some embodiments, the first gRNA targets a target site for CISH having the sequence set forth in SEQ ID NO:28 and the second gRNA targets a target site for MED12 having the sequence set forth in SEQ ID NO:81.

In some embodiments, the combination of gRNAs comprises at least three gRNAs targeting at least three different genes. In some embodiments, the combination of gRNAs comprises a first gRNA targeted to a first gene, a second gRNA targeted to a second gene, and a third gRNA targeted to a third gene. In some embodiments, the first gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2, the second gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2, the third gRNA targets a gene selected from the list consisting of CBLB, CCNC, CD5, CISH, DGKZ, ELOB, FAS, Fli1, GATA3, KDM1A, MED12, MYB, PRDM1, TGFBR2, and RASA2, and the first, second, and third gRNA each target a different gene.

In some embodiments, the combination of gRNAs targets a combination of target sites for a combination of genes for transcriptional repression, as shown in Table 2 and described in Section I.B.2.

In some embodiments, the combination of gRNAs comprises a first gRNA that targets a target site for CBLB comprising the sequence set forth in SEQ ID NO:11, and a second gRNA that targets a target site for CCNC comprising the sequence set forth in SEQ ID NO:104. In some embodiments, the combination of gRNAs comprises a first gRNA that targets a target site for CBLB comprising the sequence set forth in SEQ ID NO:11, and a second gRNA that targets a target site for CD5 comprising the sequence set forth in SEQ ID NO:3. In some embodiments, the combination of gRNAs comprises a first gRNA that targets a target site for CBLB comprising the sequence set forth in SEQ ID NO:11, and a second gRNA that targets a target site for CISH comprising the sequence set forth in SEQ ID NO:30. In some embodiments, the combination of gRNAs comprises a first gRNA that targets a target site for CBLB comprising the sequence set forth in SEQ ID NO:11, and a second gRNA that targets a target site for DGKZ comprising the sequence set forth in SEQ ID NO:13. In some embodiments, the combination of gRNAs comprises a first gRNA that targets a target site for CBLB comprising the sequence set forth in SEQ ID NO:11, and a second gRNA that targets a target site for ELOB comprising the sequence set forth in SEQ ID NO:24. In some embodiments, the combination of gRNAs comprises a first gRNA that targets a target site for CBLB comprising the sequence set forth in SEQ ID NO:11, and a second gRNA that targets a target site for FAS comprising the sequence set forth in SEQ ID NO:204. In some embodiments, the combination of gRNAs comprises a first gRNA that targets a target site for CBLB comprising the sequence set forth in SEQ ID NO:11, and a second gRNA that targets a target site for Fli1 comprising the sequence set forth in SEQ ID NO:208. In some embodiments, the combination of gRNAs comprises a first gRNA that targets a target site for CBLB comprising the sequence set forth in SEQ ID NO:11, and a second gRNA that targets a target site for GATA3 comprising the sequence set forth in SEQ ID NO:26. In some embodiments, the combination of gRNAs comprises a first gRNA that targets a target site for CBLB comprising the sequence set forth in SEQ ID NO:11, and a second gRNA that targets a target site for KDM1A comprising the sequence set forth in SEQ ID NO:4. In some embodiments, the combination of gRNAs comprises a first gRNA that targets a target site for CBLB comprising the sequence set forth in SEQ ID NO:11, and a second gRNA that targets a target site for MED12 comprising the sequence set forth in SEQ ID NO:81. In some embodiments, the combination of gRNAs comprises a first gRNA that targets a target site for CBLB comprising the sequence set forth in SEQ ID NO:11, and a second gRNA that targets a target site for MYB comprising the sequence set forth in SEQ ID NO:18. In some embodiments, the combination of gRNAs comprises a first gRNA that targets a target site for CBLB comprising the sequence set forth in SEQ ID NO:11, and a second gRNA that targets a target site for PRDM1 comprising the sequence set forth in SEQ ID NO:32. In some embodiments, the combination of gRNAs comprises a first gRNA that targets a target site for CBLB comprising the sequence set forth in SEQ ID NO:11, and a second gRNA that targets a target site for RASA2 comprising the sequence set forth in SEQ ID NO:19. In some embodiments, the combination of gRNAs comprises a first gRNA that targets a target site for CD5 comprising the sequence set forth in SEQ ID NO:3, and a second gRNA that targets a target site for CISH comprising the sequence set forth in SEQ ID NO:30. In some embodiments, the combination of gRNAs comprises a first gRNA that targets a target site for CD5 comprising the sequence set forth in SEQ ID NO:3, and a second gRNA that targets a target site for MYB comprising the sequence set forth in SEQ ID NO:18. In some embodiments, the combination of gRNAs comprises a first gRNA that targets a target site for CISH comprising the sequence set forth in SEQ ID NO:30, and a second gRNA that targets a target site for DGKZ comprising the sequence set forth in SEQ ID NO:13. In some embodiments, the combination of gRNAs comprises a first gRNA that targets a target site for CISH comprising the sequence set forth in SEQ ID NO:30, and a second gRNA that targets a target site for MYB comprising the sequence set forth in SEQ ID NO:18. In some embodiments, the combination of gRNAs comprises a first gRNA that targets a target site for CISH comprising the sequence set forth in SEQ ID NO:30, and a second gRNA that targets a target site for RASA2 comprising the sequence set forth in SEQ ID NO:19. In some embodiments, the combination of gRNAs comprises a first gRNA that targets a target site for GATA3 comprising the sequence set forth in SEQ ID NO:26, and a second gRNA that targets a target site for CD5 comprising the sequence set forth in SEQ ID NO:3. In some embodiments, the combination of gRNAs comprises a first gRNA that targets a target site for GATA3 comprising the sequence set forth in SEQ ID NO:26, and a second gRNA that targets a target site for CISH comprising the sequence set forth in SEQ ID NO:30. In some embodiments, the combination of gRNAs comprises a first gRNA that targets a target site for GATA3 comprising the sequence set forth in SEQ ID NO:26, and a second gRNA that targets a target site for MYB comprising the sequence set forth in SEQ ID NO:18. In some embodiments, the combination of gRNAs comprises a first gRNA that targets a target site for MED12 comprising the sequence set forth in SEQ ID NO:81, and a second gRNA that targets a target site for CBLB comprising the sequence set forth in SEQ ID NO:11. In some embodiments, the combination of gRNAs comprises a first gRNA that targets a target site for MED12 comprising the sequence set forth in SEQ ID NO:81, and a second gRNA that targets a target site for CD5 comprising the sequence set forth in SEQ ID NO:3. In some embodiments, the combination of gRNAs comprises a first gRNA that targets a target site for MED12 comprising the sequence set forth in SEQ ID NO:81, and a second gRNA that targets a target site for CISH comprising the sequence set forth in SEQ ID NO:30. In some embodiments, the combination of gRNAs comprises a first gRNA that targets a target site for MED12 comprising the sequence set forth in SEQ ID NO:81, and a second gRNA that targets a target site for DGKZ comprising the sequence set forth in SEQ ID NO:13. In some embodiments, the combination of gRNAs comprises a first gRNA that targets a target site for MED12 comprising the sequence set forth in SEQ ID NO:81, and a second gRNA that targets a target site for ELOB comprising the sequence set forth in SEQ ID NO:24. In some embodiments, the combination of gRNAs comprises a first gRNA that targets a target site for MED12 comprising the sequence set forth in SEQ ID NO:81, and a second gRNA that targets a target site for GATA3 comprising the sequence set forth in SEQ ID NO:26. In some embodiments, the combination of gRNAs comprises a first gRNA that targets a target site for MED12 comprising the sequence set forth in SEQ ID NO:81, and a second gRNA that targets a target site for MYB comprising the sequence set forth in SEQ ID NO:18. In some embodiments, the combination of gRNAs comprises a first gRNA that targets a target site for MED12 comprising the sequence set forth in SEQ ID NO:81, and a second gRNA that targets a target site for PRDM1 comprising the sequence set forth in SEQ ID NO:32. In some embodiments, the combination of gRNAs comprises a first gRNA that targets a target site for MED12 comprising the sequence set forth in SEQ ID NO:81, and a second gRNA that targets a target site for RASA2 comprising the sequence set forth in SEQ ID NO:19. In some embodiments, the combination of gRNAs comprises a first gRNA that targets a target site for MYB comprising the sequence set forth in SEQ ID NO:18, and a second gRNA that targets a target site for RASA2 comprising the sequence set forth in SEQ ID NO:19. In some embodiments, the combination of gRNAs comprises a first gRNA that targets a target site for PRDM1 comprising the sequence set forth in SEQ ID NO:32, and a second gRNA that targets a target site for CISH comprising the sequence set forth in SEQ ID NO:30. In some embodiments, the combination of gRNAs comprises a first gRNA that targets a target site for PRDM1 comprising the sequence set forth in SEQ ID NO:32, and a second gRNA that targets a target site for GATA3 comprising the sequence set forth in SEQ ID NO:26. In some embodiments, the combination of gRNAs comprises a first gRNA that targets a target site for PRDM1 comprising the sequence set forth in SEQ ID NO:32, and a second gRNA that targets a target site for MYB comprising the sequence set forth in SEQ ID NO:18. In some embodiments, the combination of gRNAs comprises a first gRNA that targets a target site for PRDM1 comprising the sequence set forth in SEQ ID NO:32, and a second gRNA that targets a target site for RASA2 comprising the sequence set forth in SEQ ID NO:19. In some embodiments, the combination of gRNAs comprises a first gRNA that targets a target site for CD5 comprising the sequence set forth in SEQ ID NO:3, a second gRNA that targets a target site for CISH comprising the sequence set forth in SEQ ID NO:30, and a third gRNA that targets a target site for MYB comprising the sequence set forth in SEQ ID NO:18. In some embodiments, the combination of gRNAs comprises a first gRNA that targets a target site for GATA3 comprising the sequence set forth in SEQ ID NO:26, a second gRNA that targets a target site for CBLB comprising the sequence set forth in SEQ ID NO:11, and a third gRNA that targets a target site for MYB comprising the sequence set forth in SEQ ID NO:18. In some embodiments, the combination of gRNAs comprises a first gRNA that targets a target site for GATA3 comprising the sequence set forth in SEQ ID NO:26, a second gRNA that targets a target site for CD5 comprising the sequence set forth in SEQ ID NO:3, and a third gRNA that targets a target site for MYB comprising the sequence set forth in SEQ ID NO:18. In some embodiments, the combination of gRNAs comprises a first gRNA that targets a target site for PRDM1 comprising the sequence set forth in SEQ ID NO:32, a second gRNA that targets a target site for GATA3 comprising the sequence set forth in SEQ ID NO:26, and a third gRNA that targets a target site for CISH comprising the sequence set forth in SEQ ID NO:30.

TABLE 5
Genes, target sites, and gRNAs for transcriptional repression

| | | | | | gRNA |
|---|---|---|---|---|---|
| | | | target | | spacer |
| | gRNA | target site | SEQ | gRNA spacer | SEQ |
| Gene | name | (protospacer) sequence | ID | sequence | ID |
| CBLB | CBLB_1 | GAACAGCTCGCTCCCGAA | 10 | GAACAGCUCGCUCCCGAA | 44 |
| | | GA | | GA | |
| CBLB | CBLB_2 | CGCTGGGTTGCTCCTTCTT | 11 | CGCUGGGUUGCUCCUUCU | 45 |
| | | C | | UC | |
| CBLB | CBLB_3 | CGTCCAGGCAGACGGCGG | 12 | CGUCCAGGCAGACGGCGG | 46 |
| | | TG | | UG | |
| CCNC | CCNC_1 | AAAGTTCCGGCCCGCGGT | 102 | AAAGUUCCGGCCCGCGGU | 113 |
| | | AG | | AG | |
| CCNC | CCNC_2 | GGGCCGGAACTTTTGTCGA | 103 | GGGCCGGAACUUUUGUCG | 114 |
| | | T | | AU | |
| CCNC | CCNC_3 | CGACGGCGAAAGGAAGAG | 104 | CGACGGCGAAAGGAAGAG | 115 |
| | | GA | | GA | |
| CCNC | CCNC_4 | CGAGGAGCGCGGTTACCG | 105 | CGAGGAGCGCGGUUACCG | 116 |
| | | GA | | GA | |
| CCNC | CCNC_5 | CGGCCGGCGTGAAGGAGA | 106 | CGGCCGGCGUGAAGGAGA | 117 |
| | | CT | | CU | |
| CCNC | CCNC_6 | TCACGAGAGCTCGCGGCG | 107 | UCACGAGAGCUCGCGGCG | 118 |
| | | GT | | GU | |
| CCNC | CCNC_7 | CTGGGTCTATGGTCGCTCC | 108 | CUGGGUCUAUGGUCGCUC | 119 |
| | | G | | CG | |
| CCNC | CCNC_8 | GAACTTTTGTCGATAGGAA | 109 | GAACUUUUGUCGAUAGGA | 120 |
| | | C | | AC | |
| CCNC | CCNC_9 | GCTGATTTGATCGAGGAGC | 110 | GCUGAUUUGAUCGAGGAG | 121 |
| | | G | | CG | |
| CCNC | CCNC_10 | GGAGGAGCGCGGTTACCG | 111 | GGAGGAGCGCGGUUACCG | 122 |
| | | GA | | GA | |
| CCNC | CCNC_11 | GTGGGTCTATGGTCGCTCC | 112 | GUGGGUCUAUGGUCGCUC | 123 |
| | | G | | CG | |
| CD5 | CD5_1 | TTGCACTGGAAGGGTAAA | 1 | UUGCACUGGAAGGGUAAA | 35 |
| | | GC | | GC | |
| CD5 | CD5_2 | GGAGGCGACCAAGTAAAG | 2 | GGAGGCGACCAAGUAAAG | 36 |
| | | GC | | GC | |
| CD5 | CD5_3 | GTCAGTGGGGGACCTCGC | 3 | GUCAGUGGGGGACCUCGC | 37 |
| | | AG | | AG | |
| CISH | CISH_1 | TGTCCTGCGCCCGCGCGCC | 28 | UGUCCUGCGCCCGCGCGCC | 62 |
| | | C | | C | |
| CISH | CISH_2 | GGCGGCTGGAGGGAACCA | 29 | GGCGGCUGGAGGGAACCA | 63 |
| | | GT | | GU | |
| CISH | CISH_3 | GTGGCGCGGACCGCCTGC | 30 | GUGGCGCGGACCGCCUGC | 64 |
| | | GA | | GA | |
| DGKZ | DGKZ_1 | GGGACACGGGCGGGATCG | 13 | GGGACACGGGCGGGAUCG | 47 |
| | | GT | | GU | |
| DGKZ | DGKZ_2 | GGAGCGAGCGCGCGCCAT | 14 | GGAGCGAGCGCGCGCCAU | 48 |
| | | GG | | GG | |
| DGKZ | DGKZ_3 | TCTTCGGGCACAGGTGAGC | 15 | UCUUCGGGCACAGGUGAG | 49 |
| | | G | | CG | |
| ELOB | ELOB_1 | CGAACTCCTTGGGCTAGAA | 22 | CGAACUCCUUGGGCUAGA | 56 |
| | | G | | AG | |
| ELOB | ELOB_2 | TGCGGCCGCCATCCCGACG | 23 | UGCGGCCGCCAUCCCGAC | 57 |
| | | G | | GG | |
| ELOB | ELOB_3 | CTGGAAGCGGGCGGTATC | 24 | CUGGAAGCGGGCGGUAUC | 58 |
| | | GA | | GA | |
| FAS | FAS_1 | GACCCGCTCAGTACGGAG | 200 | GACCCGCUCAGUACGGAG | 212 |
| | | TT | | UU | |
| FAS | FAS_2 | TCCCCAACTCCGTACTGAG | 201 | UCCCCAACUCCGUACUGA | 213 |
| | | C | | GC | |
| FAS | FAS_3 | GTTGGTGGACCCGCTCAGT | 202 | GUUGGUGGACCCGCUCAG | 214 |
| | | A | | UA | |
| FAS | FAS_4 | GGACCCGCTCAGTACGGA | 203 | GGACCCGCUCAGUACGGA | 215 |
| | | GT | | GU | |
| FAS | FAS_5 | ACCCGCTCAGTACGGAGTT | 204 | ACCCGCUCAGUACGGAGU | 216 |
| | | G | | UG | |
| FAS | FAS_6 | GAAGCAGTGGTTAAGCCG | 205 | GAAGCAGUGGUUAAGCCG | 217 |
| | | GA | | GA | |
| FAS | FAS_7 | TTCCCCAACTCCGTACTGA | 292 | UUCCCCAACUCCGUACUG | 296 |
| | | G | | AG | |
| FAS | FAS_8 | GGGAAGCTCTTTCACTTCG | 293 | GGGAAGCUCUUUCACUUC | 297 |
| | | G | | GG | |
| FAS | FAS_9 | ACTGTAAGTCGCTGCCTGA | 294 | ACUGUAAGUCGCUGCCUG | 298 |
| | | GTGG | | AGUGG | |
| FAS | FAS_10 | GAGCGGGTCCACCAACCC | 295 | GAGCGGGUCCACCAACCC | 299 |
| | | GC | | GC | |
| Fli1 | Fli1_1 | CGCGCGGCGGCCCAGGAG | 206 | CGCGCGGCGGCCCAGGAG | 218 |
| | | GG | | GG | |
| Fli1 | Fli1_2 | GCGCTCGCAGGGGGCACG | 207 | GCGCUCGCAGGGGGCACG | 219 |
| | | CA | | CA | |
| Fli1 | Fli1_3 | ACAACAACAAACGTGCAC | 208 | ACAACAACAAACGUGCAC | 220 |
| | | AG | | AG | |
| Fli1 | Fli1_4 | GAGGGCAGGGCGCTCGCA | 209 | GAGGGCAGGGCGCUCGCA | 221 |
| | | GG | | GG | |
| Fli1 | Fli1_5 | AAACGTGCACAGGGGAGT | 210 | AAACGUGCACAGGGGAGU | 222 |
| | | GA | | GA | |
| Fli1 | Fli1_6 | GAGCGAAAGAGACAGTTA | 211 | GAGCGAAAGAGACAGUUA | 223 |
| | | AC | | AC | |
| GATA3 | GATA3_ | CGGAGGGTACCTCTGCACC | 25 | CGGAGGGUACCUCUGCAC | 59 |
| | 1 | G | | CG | |
| GATA3 | GATA3_ | TCGACGAGGAGGCTCCAC | 26 | UCGACGAGGAGGCUCCAC | 60 |
| | 2 | CC | | CC | |
| GATA3 | GATA3_ | CAGGGCTGACTGTTACGAC | 27 | CAGGGCUGACUGUUACGA | 61 |
| | 3 | T | | CU | |
| KDM1A | KDM1A_ | CGCGCGGGCAGCGTGAAG | 4 | CGCGCGGGCAGCGUGAAG | 38 |
| | 1 | CG | | CG | |
| KDM1A | KDM1A_ | AGCGGCAGCAACCGGGAC | 5 | AGCGGCAGCAACCGGGAC | 39 |
| | 2 | GG | | GG | |
| KDM1A | KDM1A_ | GCCCAGAAGCCCTAAGAC | 6 | GCCCAGAAGCCCUAAGAC | 40 |
| | 3 | CA | | CA | |
| MED12 | Med12_1 | ACCATTGCCGGAAACTACC | 80 | ACCAUUGCCGGAAACUAC | 91 |
| | | G | | CG | |
| MED12 | Med12_2 | GTGCCCCGGGAGTTTTTCG | 81 | GUGCCCCGGGAGUUUUUC | 92 |
| | | G | | GG | |
| MED12 | Med12_3 | ACGGCGGCCGAGAGACAA | 82 | ACGGCGGCCGAGAGACAA | 93 |
| | | CA | | CA | |
| MED12 | Med12_4 | CGAGGTACGCCGGGAACC | 83 | CGAGGUACGCCGGGAACC | 94 |
| | | AT | | AU | |
| MED12 | Med12_5 | CGCCACCGCCGAAAAACT | 84 | CGCCACCGCCGAAAAACU | 95 |
| | | CC | | CC | |
| MED12 | Med12_6 | CGATGGTTCCCGGCGTACC | 85 | CGAUGGUUCCCGGCGUAC | 96 |
| | | T | | CU | |
| MED12 | Med12_7 | CGGCGGCCGAGAGACAAC | 86 | CGGCGGCCGAGAGACAAC | 97 |
| | | AA | | AA | |
| MED12 | Med12_8 | TCCTGAGGGTAAACATCG | 87 | UCCUGAGGGUAAACAUCG | 98 |
| | | GG | | GG | |
| MED12 | Med12_9 | TTCGTAGCTCAAGATCCCG | 88 | UUCGUAGCUCAAGAUCCC | 99 |
| | | A | | GA | |
| MED12 | Med12_ | GCTGACTGGGGGAACGGG | 89 | GCUGACUGGGGGAACGGG | 100 |
| | 10 | AA | | AA | |
| MED12 | Med12_ | GGCTGGTGCCTCCGGCGCT | 90 | GGCUGGUGCCUCCGGCGC | 101 |
| | 11 | A | | UA | |
| MYB | MYB_1 | GCCGAATGGGAGCGGCGA | 16 | GCCGAAUGGGAGCGGCGA | 50 |
| | | CC | | CC | |
| MYB | MYB_2 | GGATCCCTCGCCGACACCC | 17 | GGAUCCCUCGCCGACACCC | 51 |
| | | G | | G | |
| MYB | MYB_3 | GAAACTTCGCCCCAGCGGT | 18 | GAAACUUCGCCCCAGCGG | 52 |
| | | G | | UG | |
| PRDM1 | PRDM1_ | AGAGGCAAGAGCAGCGAC | 31 | AGAGGCAAGAGCAGCGAC | 65 |
| | 1 | CG | | CG | |
| PRDM1 | PRDM1_ | GACGCGGGGAGAATGTGG | 32 | GACGCGGGGAGAAUGUGG | 66 |
| | 2 | AC | | AC | |
| PRDM1 | PRDM1_ | TTGCCTCTCCGCAACACTG | 33 | UUGCCUCUCCGCAACACU | 67 |
| | 3 | G | | GG | |
| RASA2 | RASA2_1 | GCACGGGCCGGGCGGCAC | 19 | GCACGGGCCGGGCGGCAC | 53 |
| | | CA | | CA | |
| RASA2 | RASA2_2 | TGCTGCGGCGGCTTCTTCC | 20 | UGCUGCGGCGGCUUCUUC | 54 |
| | | G | | CG | |
| RASA2 | RASA2_3 | GCGCTGGGCGCGAGGCTG | 21 | GCGCUGGGCGCGAGGCUG | 55 |
| | | AG | | AG | |
| TGFBR2 | TGFBR2_ | ACTTCAACTCAGCGCTGCG | 300 | ACUUCAACUCAGCGCUGC | 303 |
| | 1 | G | | GG | |
| TGFBR2 | T2GFBR2_ | AGTCCGGCTCCTGTCCCGA | 301 | AGUCCGGCUCCUGUCCCG | 304 |
| | 2 | G | | AG | |
| TGFBR2 | TGFBR2_ | GAAACTCCTCGCCAACAG | 302 | GAAACUCCUCGCCAACAG | 305 |
| | 3 | CT | | CU | |
| TGFBR2 | TGFBR2_ | GTCCCGAGCGGGTGCACG | 306 | GUCCCGAGCGGGUGCACG | 309 |
| | 4 | CG | | CG | |
| TGFBR2 | TGFBR2_ | GTCCGGCTCCTGTCCCGAG | 307 | GUCCGGCUCCUGUCCCGA | 310 |
| | 5 | C | | GC | |
| TGFBR2 | TGFBR2_ | CCCGAGCGGGTGCACGCG | 308 | CCCGAGCGGGUGCACGCG | 311 |
| | 6 | CG | | CG | |

b. gRNAs for Transcriptional Activation

In some embodiments, a gRNA provided herein targets a target site for a gene for transcriptional activation, such as any targets site or target gene described in Section I.B.3. In some embodiments, a gRNA provided herein targets a target site for a gene for transcriptional activation. In some embodiments, a gRNA provided herein targets a target site for a gene, such as a gene in a T cell, wherein the gene is selected from the list shown in Table 6, consisting of: BATF, CD28, EOMES, IL-2, IL2RB, IRF4, LAT, LCP2, TBX21, and VAV1.

In some embodiments, the gRNA targets a target site that comprises a sequence selected from any one of SEQ ID NOS:7-9, 78, 144-156, 170, 172-177, and 184-191, as shown in Table 6, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NOS:7-9, 78, 144-156, 170, 172-177, and 184-191 that is 14, 15, 16, 17, 18 or 19 nucleotides in length. In some embodiments, the target site is set forth in any one of SEQ ID NOS: 7-9, 78, 144-156, 170, 172-177, and 184-191.

In some embodiments, the gRNA comprises a spacer sequence selected from any one of SEQ ID NOS:41-43, 79, 157-169, 171, 178-183, and 192-199, as shown in Table 6, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS:41-43, 79, 157-169, 171, 178-183, and 192-199 that is 14, 15, 16, 17, 18 or 19 nucleotides in length. In some embodiments, the spacer sequence of the gRNA is set forth in any one of SEQ ID NOS:41-43, 79, 157-169, 171, 178-183, and 192-199.

In some embodiments, the gRNA targets a target site of IL-2. In some embodiments, the gRNA targets a target site that comprises SEQ ID NO:78, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NO: 78 that is 14, 15, 16, 17, 18 or 19 nucleotides in length. In some embodiments, the target site is set forth in any one of SEQ ID NO: 78. In some embodiments, the gRNA comprises a spacer sequence comprising SEQ ID NO:79, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 79 that is 14, 15, 16, 17, 18 or 19 nucleotides in length. In some embodiments, the spacer sequence of the gRNA is set forth in SEQ ID NO:79.

In some embodiments, the gRNA targets a target site of EOMES. In some embodiments, the gRNA targets a target site that comprises SEQ ID NO:149, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NO: 149 that is 14, 15, 16, 17, 18 or 19 nucleotides in length. In some embodiments, the target site is set forth in any one of SEQ ID NO: 149. In some embodiments, the gRNA comprises a spacer sequence comprising SEQ ID NO:162, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 162 that is 14, 15, 16, 17, 18 or 19 nucleotides in length. In some embodiments, the spacer sequence of the gRNA is set forth in SEQ ID NO:162.

In some embodiments, the gRNA targets a target site of LCP2. In some embodiments, the gRNA targets a target site that comprises SEQ ID NO:151, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NO: 151 that is 14, 15, 16, 17, 18 or 19 nucleotides in length. In some embodiments, the target site is set forth in any one of SEQ ID NO: 151. In some embodiments, the gRNA comprises a spacer sequence comprising SEQ ID NO:164, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 164 that is 14, 15, 16, 17, 18 or 19 nucleotides in length. In some embodiments, the spacer sequence of the gRNA is set forth in SEQ ID NO:164.

In some embodiments, the gRNA targets a target site of TBX21. In some embodiments, the gRNA targets a target site that comprises SEQ ID NO:155, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NO: 155 that is 14, 15, 16, 17, 18 or 19 nucleotides in length. In some embodiments, the target site is set forth in any one of SEQ ID NO: 155. In some embodiments, the gRNA comprises a spacer sequence comprising SEQ ID NO:168, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of SEQ ID NO: 168 that is 14, 15, 16, 17, 18 or 19 nucleotides in length. In some embodiments, the spacer sequence of the gRNA is set forth in SEQ ID NO:168.

In some embodiments, provided herein is a combination of gRNAs that each target a target site for a gene for transcriptional activation. In some embodiments, provided herein is a multiplexed epigenetic-modifying DNA-targeting system comprising the combination of gRNAs.

In some embodiments, the combination of gRNAs comprises at least two gRNAs targeting at least two different genes for transcriptional activation. In some embodiments, the gRNAs target a combination of genes selected from the combinations of genes listed in Table 3. In some embodiments, each gRNA of the combination of gRNAs is selected from any of the gRNAs described herein for targeted transcriptional activation.

In some embodiments, the combination of gRNAs comprises a first gRNA targeted to a first gene and a second gRNA targeted to a second gene. In some embodiments, the first gRNA targets a gene selected from the list consisting of BATF, CD28, EOMES, IL-2, IL2RB, IRF4, LAT, LCP2, TBX21, and VAV1, the second gRNA targets a gene selected from the list consisting of BATF, CD28, EOMES, IL-2, IL2RB, IRF4, LAT, LCP2, TBX21, and VAV1, and the first and second gRNAs target different genes. In some embodiments, the first gRNA targets IL-2, and the second gRNA targets a gene selected from the list consisting of BATF, CD28, EOMES, IL-2, IL2RB, IRF4, LAT, LCP2, TBX21, and VAV1. In some embodiments, the first gRNA targets VAV1, and the second gRNA targets a gene selected from the list consisting of BATF, CD28, EOMES, IL-2, IL2RB, IRF4, LAT, LCP2, TBX21, and VAV1.

In some embodiments, the first gRNA targets IL-2 and the second gRNA targets VAV1. In some embodiments, the gRNA targeting a target site of IL-2 can be any as described, and the the gRNA targeting a target site of VAV1 can be any as described. In some embodiments, the first gRNA targets a target site for IL-2 having the sequence set forth in SEQ ID NO:78 and the second gRNA targets a target site for VAV1 having the sequence set forth in SEQ ID NO:170.

In some embodiments, the first gRNA targets IL-2 and the second gRNA targets LCP2. In some embodiments, the gRNA targeting a target site of IL-2 can be any as described, and the the gRNA targeting a target site of LCP2 can be any as described. In some embodiments, the first gRNA targets a target site for IL-2 having the sequence set forth in SEQ ID NO:78 and the second gRNA targets a target site for VAV1 having the sequence set forth in SEQ ID NO:151.

In some embodiments, the first gRNA targets IL-2 and the second gRNA targets TBX21. In some embodiments, the gRNA targeting a target site of IL-2 can be any as described, and the the gRNA targeting a target site of TBX21 can be any as described. In some embodiments, the first gRNA targets a target site for IL-2 having the sequence set forth in SEQ ID NO:78 and the second gRNA targets a target site for TBX21 having the sequence set forth in SEQ ID NO:155.

In some embodiments, the first gRNA targets IL-2 and the second gRNA targets EOMES. In some embodiments, the gRNA targeting a target site of IL-2 can be any as described, and the the gRNA targeting a target site of EOMES can be any as described. In some embodiments, the first gRNA targets a target site for IL-2 having the sequence set forth in SEQ ID NO:78 and the second gRNA targets a target site for EOMES having the sequence set forth in SEQ ID NO:149.

In some embodiments, the combination of gRNAs comprises at least three gRNAs targeting at least three different genes. In some embodiments, the combination of gRNAs comprises a first gRNA targeted to a first gene, a second gRNA targeted to a second gene, and a third gRNA targeted to a third gene. In some embodiments, the first gRNA targets a gene selected from the list consisting of BATF, CD28, EOMES, IL-2, IL2RB, IRF4, LAT, LCP2, TBX21, and VAV1, the second gRNA targets a gene selected from the list consisting of BATF, CD28, EOMES, IL-2, IL2RB, IRF4, LAT, LCP2, TBX21, and VAV1, the third gRNA targets a gene selected from the list consisting of BATF, CD28, EOMES, IL-2, IL2RB, IRF4, LAT, LCP2, TBX21, and VAV1, and the first, second, and third gRNA each target a different gene.

In some embodiments, the combination of gRNAs targets a combination of target sites for a combination of genes for transcriptional activation, as shown in Table 4 and described in Section I.B.3.

In some embodiments, the combination of gRNAs comprises a first gRNA that targets a target site for BATF comprising the sequence set forth in SEQ ID NO:172, and a second gRNA that targets a target site for IL-2 comprising the sequence set forth in SEQ ID NO:78. In some embodiments, the combination of gRNAs comprises a first gRNA that targets a target site for BATF comprising the sequence set forth in SEQ ID NO:172, and a second gRNA that targets a target site for VAV1 comprising the sequence set forth in SEQ ID NO:170. In some embodiments, the combination of gRNAs comprises a first gRNA that targets a target site for CD28 comprising the sequence set forth in SEQ ID NO:144, and a second gRNA that targets a target site for BATF comprising the sequence set forth in SEQ ID NO:172. In some embodiments, the combination of gRNAs comprises a first gRNA that targets a target site for CD28 comprising the sequence set forth in SEQ ID NO:144, and a second gRNA that targets a target site for EOMES comprising the sequence set forth in SEQ ID NO:149. In some embodiments, the combination of gRNAs comprises a first gRNA that targets a target site for CD28 comprising the sequence set forth in SEQ ID NO:144, and a second gRNA that targets a target site for IL-2 comprising the sequence set forth in SEQ ID NO:78. In some embodiments, the combination of gRNAs comprises a first gRNA that targets a target site for CD28 comprising the sequence set forth in SEQ ID NO:144, and a second gRNA that targets a target site for LCP2 comprising the sequence set forth in SEQ ID NO:151. In some embodiments, the combination of gRNAs comprises a first gRNA that targets a target site for CD28 comprising the sequence set forth in SEQ ID NO:144, and a second gRNA that targets a target site for TBX21 comprising the sequence set forth in SEQ ID NO:155. In some embodiments, the combination of gRNAs comprises a first gRNA that targets a target site for CD28 comprising the sequence set forth in SEQ ID NO:144, and a second gRNA that targets a target site for VAV1 comprising the sequence set forth in SEQ ID NO:170. In some embodiments, the combination of gRNAs comprises a first gRNA that targets a target site for EOMES comprising the sequence set forth in SEQ ID NO:149, and a second gRNA that targets a target site for BATF comprising the sequence set forth in SEQ ID NO:172. In some embodiments, the combination of gRNAs comprises a first gRNA that targets a target site for EOMES comprising the sequence set forth in SEQ ID NO:149, and a second gRNA that targets a target site for LCP2 comprising the sequence set forth in SEQ ID NO:151. In some embodiments, the combination of gRNAs comprises a first gRNA that targets a target site for EOMES comprising the sequence set forth in SEQ ID NO:149, and a second gRNA that targets a target site for TBX21 comprising the sequence set forth in SEQ ID NO:155. In some embodiments, the combination of gRNAs comprises a first gRNA that targets a target site for EOMES comprising the sequence set forth in SEQ ID NO:149, and a second gRNA that targets a target site for VAV1 comprising the sequence set forth in SEQ ID NO:170. In some embodiments, the combination of gRNAs comprises a first gRNA that targets a target site for LCP2 comprising the sequence set forth in SEQ ID NO:151, and a second gRNA that targets a target site for BATF comprising the sequence set forth in SEQ ID NO:172. In some embodiments, the combination of gRNAs comprises a first gRNA that targets a target site for LCP2 comprising the sequence set forth in SEQ ID NO:151, and a second gRNA that targets a target site for IL-2 comprising the sequence set forth in SEQ ID NO:78. In some embodiments, the combination of gRNAs comprises a first gRNA that targets a target site for LCP2 comprising the sequence set forth in SEQ ID NO:151, and a second gRNA that targets a target site for TBX21 comprising the sequence set forth in SEQ ID NO:155. In some embodiments, the combination of gRNAs comprises a first gRNA that targets a target site for LCP2 comprising the sequence set forth in SEQ ID NO:151, and a second gRNA that targets a target site for VAV1 comprising the sequence set forth in SEQ ID NO:170. In some embodiments, the combination of gRNAs comprises a first gRNA that targets a target site for TBX21 comprising the sequence set forth in SEQ ID NO:155, and a second gRNA that targets a target site for BATF comprising the sequence set forth in SEQ ID NO:172. In some embodiments, the combination of gRNAs comprises a first gRNA that targets a target site for TBX21 comprising the sequence set forth in SEQ ID NO:155, and a second gRNA that targets a target site for IL-2 comprising the sequence set forth in SEQ ID NO:78. In some embodiments, the combination of gRNAs comprises a first gRNA that targets a target site for TBX21 comprising the sequence set forth in SEQ ID NO:155, and a second gRNA that targets a target site for TBX21 comprising the sequence set forth in SEQ ID NO:155. In some embodiments, the combination of gRNAs comprises a first gRNA that targets a target site for TBX21 comprising the sequence set forth in SEQ ID NO:155, and a second gRNA that targets a target site for VAV1 comprising the sequence set forth in SEQ ID NO:170. In some embodiments, the combination of gRNAs comprises a first gRNA that targets a target site for VAV1 comprising the sequence set forth in SEQ ID NO:170, and a second gRNA that targets a target site for IL-2 comprising the sequence set forth in SEQ ID NO:78.

TABLE 6
Genes, target sites, and gRNAs for transcriptional activation

| | | | | | gRNA |
|---|---|---|---|---|---|
| | | | target | | spacer |
| | gRNA | target site | SEQ | gRNA spacer | SEQ |
| Gene | name | (protospacer) sequence | ID | sequence | ID |
| BATF | BATF_1 | ACTCACGCTGGAAGTCAC | 172 | ACUCACGCUGGAAGUCAC | 178 |
| | | AT | | AU | |
| BATF | BATF_2 | AAGTCCGTCTTCTGTCAAC | 173 | AAGUCCGUCUUCUGUCAA | 179 |
| | | A | | CA | |
| BATF | BATF_3 | GCAGAGGGACTGCTCCCC | 174 | GCAGAGGGACUGCUCCCC | 180 |
| | | AA | | AA | |
| CD28 | CD28_1 | CTGACTGCAGCATTTCAC | 144 | CUGACUGCAGCAUUUCAC | 157 |
| | | AC | | AC | |
| CD28 | CD28_2 | GCTGCAGTCAGGATGCCT | 145 | GCUGCAGUCAGGAUGCCU | 158 |
| | | TG | | UG | |
| CD28 | CD28_3 | CCTTGATCATGTGCCCTAA | 146 | CCUUGAUCAUGUGCCCUA | 159 |
| | | G | | AG | |
| CD28 | CD28_4 | TCGTCAGGACAAAGATGC | 189 | UCGUCAGGACAAAGAUGC | 197 |
| | | TC | | UC | |
| CD28 | CD28_5 | CGTGGATGACGGAGACTC | 190 | CGUGGAUGACGGAGACUC | 198 |
| | | TC | | UC | |
| CD28 | CD28_6 | CTGAGAGTCTCCGTCATC | 191 | CUGAGAGUCUCCGUCAUC | 199 |
| | | CA | | CA | |
| EOMES | EOMES_ | CGCAGTAGCGGCCCGCGA | 147 | CGCAGUAGCGGCCCGCGA | 160 |
| | 1 | GT | | GU | |
| EOMES | EOMES_ | CGGGCCGCTACTGCGCGT | 148 | CGGGCCGCUACUGCGCGU | 161 |
| | 2 | AC | | AC | |
| EOMES | EOMES_ | GCTACCCACCTGCCGACT | 149 | GCUACCCACCUGCCGACU | 162 |
| | 3 | CG | | CG | |
| IL-2 | IL-2_1 | GAGAGCTATCACCTAAGT | 78 | GAGAGCUAUCACCUAAGU | 79 |
| | | GT | | GU | |
| IRF4 | IRF4_1 | CCGCTTCGGGGACTGTCA | 175 | CCGCUUCGGGGACUGUCA | 181 |
| | | CT | | CU | |
| IRF4 | IRF4_2 | ACTTTGCAAGCCGAGAGC | 176 | ACUUUGCAAGCCGAGAGC | 182 |
| | | CG | | CG | |
| IRF4 | IRF4_3 | GTCCAACCCCCGGCCCCC | 177 | GUCCAACCCCCGGCCCCC | 183 |
| | | AC | | AC | |
| LAT | LAT_1 | GGCTGGGACGCAGGGGTA | 184 | GGCUGGGACGCAGGGGUA | 192 |
| | | AC | | AC | |
| LAT | LAT_2 | CCACCCCAGGCACTCACC | 185 | CCACCCCAGGCACUCACC | 193 |
| | | AA | | AA | |
| LAT | LAT_3 | CTGTGGTGAGCGCCGGGC | 186 | CUGUGGUGAGCGCCGGGC | 194 |
| | | GA | | GA | |
| LCP2 | LCP2_1 | TCCAGTCACCCCAACCCA | 150 | UCCAGUCACCCCAACCCA | 163 |
| | | GT | | GU | |
| LCP2 | LCP2_2 | GGTGCTACACTGTGCAGA | 151 | GGUGCUACACUGUGCAGA | 164 |
| | | CA | | CA | |
| LCP2 | LCP2_3 | CAGGGTTGGTAATTCCCC | 152 | CAGGGUUGGUAAUUCCCC | 165 |
| | | AC | | AC | |
| LCP2 | LCP2_4 | AAAGACATCGGCTCCAAC | 187 | AAAGACAUCGGCUCCAAC | 195 |
| | | AG | | AG | |
| LCP2 | LCP2_5 | AGCCGTTGCTTTCTGGGAT | 188 | AGCCGUUGCUUUCUGGGA | 196 |
| | | C | | UC | |
| TBX21 | TBX21_1 | CAGGGCCGAGGTGGCGGA | 153 | CAGGGCCGAGGUGGCGGA | 166 |
| | | GT | | GU | |
| TBX21 | TBX21_2 | CTCGCTTCTCTCCACCATG | 154 | CUCGCUUCUCUCCACCAU | 167 |
| | | G | | GG | |
| TBX21 | TBX21_3 | CTAGGCAAATTCTACGCT | 155 | CUAGGCAAAUUCUACGCU | 168 |
| | | CT | | CU | |
| VAV1 | VAV1_1 | TGTCGCTCCACAGGCGAG | 7 | UGUCGCUCCACAGGCGAG | 41 |
| | | CA | | CA | |
| VAV1 | VAV1_2 | CCTCTCAGGGCGACAGTT | 8 | CCUCUCAGGGCGACAGUU | 42 |
| | | AC | | AC | |
| VAV1 | VAV1_3 | GGCCAGCTAGACTATGAG | 9 | GGCCAGCUAGACUAUGAG | 43 |
| | | AT | | AU | |
| VAV1 | VAV1_4 | CCTGTAACTGTCGCCCTG | 156 | CCUGUAACUGUCGCCCUG | 169 |
| | | AG | | AG | |
| VAV1 | VAV1_5 | CCAGGCCTGTGTCGAGTG | 170 | CCAGGCCUGUGUCGAGUG | 171 |
| | | GG | | GG | |

### D. Other DNA-Binding Domains and DNA-Targeting Systems

In some of any of the provided embodiments, the DNA-binding domain comprises a zinc finger protein (ZFP); a transcription activator-like effector (TALE); a meganuclease; a homing endonuclease; or an I-SceI enzyme or a variant thereof. In some embodiments, the DNA-binding domain comprises a catalytically inactive variant of any of the foregoing. In some embodiments, the fusion protein of the DNA-targeting system, or one or more DNA-targeting modules thereof, comprises a DNA-binding domain described herein, such as a DNA-binding domain that is an engineered zinc finger protein (eZFP) or a TALE.

In some embodiments, a ZFP, a zinc finger DNA binding protein, or zinc finger DNA binding domain, is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial, or engineered ZFPs (eZFPs), comprising ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is an eZFP that is engineered to bind to a target site of choice.

In some embodiments, zinc fingers are custom-designed (i.e. designed by the user), or obtained from a commercial source. Various methods for designing zinc finger proteins are available. For example, methods for designing zinc finger proteins to bind to a target DNA sequence of interest are described, for example in Liu, Q. et al., PNAS, 94(11):5525-30 (1997); Wright, D. A. et al., Nat. Protoc., 1(3):1637-52 (2006); Gersbach, C. A. et al., Acc. Chem. Res., 47(8):2309-18 (2014); Bhakta M. S. et al., Methods Mol. Biol., 649:3-30 (2010); and Gaj et al., Trends Biotechnol, 31(7):397-405 (2013). In addition, various web-based tools for designing zinc finger proteins to bind to a DNA target sequence of interest are publicly available. See, for example, the Zinc Finger Tools design web site from Scripps available on the world wide web at scripps.edu/barbas/zfdesign/zfdesignhome.php. Various commercial services for designing zinc finger proteins to bind to a DNA target sequence of interest are also available. See, for example, the commercially available services or kits offered by Creative Biolabs (world wide web at creative-biolabs.com/Design-and-Synthesis-of-Artificial-Zinc-Finger-Proteins.html), the Zinc Finger Consortium Modular Assembly Kit available from Addgene (world wide web at addgene.org/kits/zfc-modular-assembly/), or the CompoZr Custom ZFN Service from Sigma Aldrich (world wide web at sigmaaldrich.com/life-science/zinc-finger-nuclease-technology/custom-zfn.html).

In some embodiments, the fusion protein of the DNA-targeting system comprises an eZFP DNA-binding domain and an effector domain.

Transcription activator-like effectors (TALEs), are proteins naturally found in Xanthomonas bacteria. TALEs comprise a plurality of repeated amino acid sequences, each repeat having binding specificity for one base in a target sequence. Each repeat comprises a pair of variable residues in position 12 and 13 (repeat variable diresidue; RVD) that determine the nucleotide specificity of the repeat. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, RVDs can be mutated towards other amino acid residues in order to modulate their specificity towards nucleotides A, T, C and G and in particular to enhance this specificity. Binding domains with similar modular base-per-base nucleic acid binding properties can also be derived from different bacterial species. These alternative modular proteins may exhibit more sequence variability than TALE repeats.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, the fusion protein of the DNA-targeting system comprises a TALE DNA-binding domain and an effector domain.

Zinc finger and TALE DNA-binding domains can be engineered to bind to a predetermined nucleotide sequence, for example via engineering (altering one or more amino acids) of the recognition helix region of a naturally occurring zinc finger protein, by engineering of the amino acids in a TALE repeat involved in DNA binding (the repeat variable diresidue or RVD region), or by systematic ordering of modular DNA-binding domains, such as TALE repeats or ZFP domains. Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

### E. Effector Domains

In some aspects, the DNA-targeting systems provided herein further include one or more effector domains. In some embodiments, the one or more effector domains are transcriptional repressor effector domains. In some embodiments, in a DNA-targeting system having a plurality of effector domains, each effector domain is a transcriptional repressor. In some embodiments, the one or more effector domains are transcriptional activator effector domains. In some embodiments, in a DNA-targeting system having a plurality of effector domains, each effector domain is a transcriptional activator. In some embodiments, provided herein is a DNA-targeting system comprising a fusion protein comprising: (a) a DNA-binding domain capable of being targeted to a target site in a gene or regulatory DNA element thereof, such as any described above in Section I.C or Section I.D, and (b) at least one effector domain.

### 1. Effector Domains for Transcriptional Repression

In some aspects, the DNA-targeting systems provided herein further include one or more effector domains, such as a transcriptional repressor effector domain. In some embodiments, provided herein is a DNA-targeting system comprising a fusion protein comprising: (a) a DNA-binding domain capable of being targeted to a target site in a gene or regulatory DNA element thereof, such as any DNA-binding domain described above in Section I.C or Section I.D, and (b) at least one effector domain. In some aspects, the effector domain is capable of reducing transcription of the gene or combination of genes, such as any of the genes described in Section I.B.2. In some aspects, the effector domain comprises a transcription repressor domain.

In some aspects, the effector domain induces, catalyzes, or leads to repressed and/or reduced transcription of a gene when ectopically recruited to the gene or DNA regulatory element thereof.

In some embodiments, the effector domain induces, catalyzes or leads to transcription repression, transcription co-repression, histone modification, histone acetylation, histone deacetylation, nucleosome remodeling, chromatin remodeling, heterochromatin formation, proteolysis, ubiquitination, deubiquitination, phosphorylation, dephosphorylation, splicing, DNA methylation, DNA demethylation, histone methylation, histone demethylation, or DNA base oxidation. In some embodiments, the effector domain induces, catalyzes, or leads to transcription repression or transcription co-repression. In some embodiments, the effector domain induces transcription repression. In some embodiments, the effector domain has one of the aforementioned activities itself (i.e. acts directly). In some embodiments, the effector domain recruits and/or interacts with a protein or polypeptide domain that has one of the aforementioned activities (i.e. acts indirectly).

Gene expression of endogenous mammalian genes, such as human genes, can be achieved by targeting a fusion protein comprising a DNA-binding domain, such as a dCas9, and an effector domain, such as a transcription repression domain, to mammalian genes or regulatory DNA elements thereof (e.g. a promoter or enhancer) via one or more gRNAs. Any of a variety of effector domains for transcriptional repression (e.g. transcription repression domains) are known and can be used in accord with the provided embodiments. Transcription repression domains, as well as transcriptional repression of target genes using Cas fusion proteins with the transcription repression domains, are described, for example, in WO 2014/197748, WO 2017/180915, WO 2021/226077, WO 2013/176772, WO 2014/152432, WO 2014/093661, Adli, M. Nat. Commun. 9, 1911 (2018), and Gilbert, L. A. et al. Cell 154(2):442-451 (2013).

In some embodiments, the effector domain may comprise a KRAB domain, ERF repressor domain, MXII domain, SID4X domain, MAD-SID domain, a DNMT family protein domain (e.g. DNMT3A or DNMT3B), a fusion of one or more DNMT family proteins or domains thereof (e.g. DNMT3A/L, which comprises a fusion of DNMT3A and DNMT3L domains), LSD1, EZH2, a SunTag domain, a partially or fully functional fragment or domain of any of the foregoing, or a combination of any of the foregoing. For example, the fusion protein may be dCas9-KRAB, or dCas9-KRAB-DNMT3A/3L. In some embodiments, the fusion protein may be dCas9-KRAB, such as dSpCas9-KRAB (e.g. SEQ ID NO:73). In some embodiments, the fusion protein may be dCas9-KRAB-DNMT3A/3L (e.g. SEQ ID NO:75).

In some embodiments, the effector domain comprises a transcriptional repressor domain described in WO 2021/226077.

In some embodiments, the effector domain comprises a KRAB domain, or a variant thereof. The KRAB-containing zinc finger proteins make up the largest family of transcriptional repressors in mammals. The Kruppel associated box (KRAB) domain is a transcriptional repressor domain present in many zinc finger protein-based transcription factors. The KRAB domain comprises charged amino acids and can be divided into sub-domains A and B. The KRAB domain recruits corepressors KAP1 (KRAB-associated protein-1), epigenetic readers such as heterochromatin protein 1 (HP1), and other chromatin modulators to induce transcriptional repression through heterochromatin formation. KRAB-mediated gene repression is associated with loss of histone H3-acetylation and an increase in H3 lysine 9 trimethylation (H3K9me3) at the repressed gene promoters. KRAB domains, including in dCas fusion proteins, have been described, for example, in WO 2017/180915, WO 2014/197748, US 2019/0127713, WO 2013/176772, Urrutia R. et al. Genome Biol. 4, 231 (2003), Groner A. C. et al. pLoS Genet. 6, e1000869 (2010). In some embodiments, the effector domain comprises at least one KRAB domain or a variant thereof. In some embodiments, an exemplary KRAB domain is set forth in SEQ ID NO:70. In some embodiments, the effector domain comprises the sequence set forth in SEQ ID NO:70, or a portion thereof, or an amino acid sequence that has at least 90%, 91%, 92%, 93%, 94%, 95%, 96%, 97%, 98%, or 99% sequence identity to SEQ ID NO:70. In some embodiments, an exemplary KRAB domain is set forth in SEQ ID NO:235. In some embodiments, the effector domain comprises the sequence set forth in SEQ ID NO:235, or a portion thereof, or an amino acid sequence that has at least 90%, 91%, 92%, 93%, 94%, 95%, 96%, 97%, 98%, or 99% sequence identity to SEQ ID NO:235.

In some embodiments, the effector domain comprises at least one ERF repressor domain, or a variant thereof. ERF (ETS2 repressor factor) is a strong transcriptional repressor that comprises a conserved ets-DNA-binding domain, and represses transcription via a distinct domain at the carboxyl-terminus of the protein. ERF repressor domains, including in dCas fusion proteins, have been described, for example, in WO2017180915, WO2014197748, WO2013176772, Mavrothalassitis, G., Ghysdael, J. Proteins of the ETS family with transcriptional repressor activity. Oncogene 19, 6524-6532 (2000). In some embodiments, the effector domain comprises at least one ERF repressor domain or a variant thereof. An exemplary ERF repressor domain is set forth in SEQ ID NO:128. In some embodiments, the effector domain comprises the sequence set forth in SEQ ID NO:128, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one MXI1 domain, or a variant thereof. The MXI1 domain functions by antagonizing the myc transcriptional activity by competing for binding to myc-associated factor x (MAX). MXI1 domains, including in dCas fusion proteins, have been described, for example, in WO2017180915, WO2014197748, US20190127713. In some embodiments, the effector domain comprises at least one MXI1 domain or a variant thereof. An exemplary MXI1 domain is set forth in SEQ ID NO:129. In some embodiments, the effector domain comprises the sequence set forth in SEQ ID NO:129, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one SID4X domain, or a variant thereof. The mSin3 interacting domain (SID) is present on different transcription repressor proteins. It interacts with the paired amphipathic alpha-helix 2 (PAH2) domain of mSin3, a transcriptional repressor domain that is attached to transcription repressor proteins such as the mSin3 A corepressor. A dCas9 molecule can be fused to four concatenated mSin3 interaction domains (SID4X). SID domains, including in dCas fusion proteins, have been described, for example, in WO2017180915, WO2014197748, WO2014093655. In some embodiments, the effector domain comprises at least one SID domain or a variant thereof. An exemplary SID domain is set forth in SEQ ID NO:236. In some embodiments, the effector domain comprises the sequence set forth in SEQ ID NO:236, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one MAD domain, or a variant thereof. The MAD family proteins, Mad1, Mxil, Mad3, and Mad4, belong to the basic helix-loop-helix-zipper class and contain a conserved N terminal region (termed Sin3 interaction domain (SID)) necessary for repressional activity. MAD-SID domains, including in dCas fusion proteins, have been described, for example, in WO2017180915, WO2014197748, WO2013176772. In some embodiments, the effector domain comprises at least one MAD-SID domain or a variant thereof. An exemplary MAD-SID domain is set forth in SEQ ID NO:237. In some embodiments, the effector domain comprises the sequence set forth in SEQ ID NO:237, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one DNMT3 domain, or a variant thereof. In some embodiments, the at least one DNMT3 domain, or a variant thereof, is from a DNMT3 or is a portion or a functionally active variant thereof with DNA methyltransferase activity. The DNMT3A and DNMT3B are two DNA methyltransferases that catalyze de novo methylation, which depending on the site may be associated with transcriptional repression. DNMTs, such as DNMT3s, mediate transfer of a methyl group from the universal methyl donor, S-adenosyl-L-methionine (SAM), to the 5-position of cytosine residues. In some aspects, these DNMT3 DNA methyltransferases induce de novo methylation of a cytosine base to methylated 5-methylcytosine. DNMT3, including in dCas fusion proteins, have been described, for example, in US20190127713, Liu, X. S. et al. Cell 167, 233-247.e17 (2016), Lei, Y. et al. Nat. Commun. 8, 16026 (2017). DNMT3 proteins, such as DNMT3A and DNMT3B, contain an N-terminal part that is naturally involved in regulatory activity and targeting, and a C-terminal catalytic domain termed the mTase C5-type domain. In some embodiments, an effector domain in embodiments provided herein includes a catalytically active portion of a DNMT3A or a DNMT3B that contains a catalytically active C-terminal domain. In particular, isolated catalytic domains of DNMT3a and DNMT3b are catalytically active (see e.g. Gowher and Jeltsch (2002) J. Biol. Chem., 277:20409).

In some embodiments, the effector domain comprises at least one DNMT3 domain or a variant thereof. In some embodiments, the DNMT3 domain may be an effector domain of DNMT3A or DNMT3B that is catalytically active. In some embodiments, the effector domain may be the full-length of DNMT3A or DNMT3B or a catalytically active portion thereof. In some embodiments, the effector domain is a catalytically active portion that is less than the full-length sequence of DNMT3A or DNMT3B. In some embodiments, a catalytically active portion is a contiguous sequence of amino acids that confers DNA methyltransferase activity, such as by mediating methylation of a cytosine base to methylated 5-methylcytosine. In some embodiments, the contiguous sequence of amino acids is a contiguous C-terminal portion of a DNMT3 protein, such as DNMT3A, or DNMT3B, that is from 280 amino acids to 330 amino acids in length. In some embodiments, the contiguous portion is 280 amino acids, 290 amino acids, 300 amino acids, 310 amino acids, 320 amino acids, or 330 amino acids in length, or is a length of any value between any of the foregoing. In some embodiments, a catalytically active portion of a DNMT, such as a DNMT3, includes a SAM-dependent mTase C5-type domain. In some embodiments, the DNMT3 domain, such as a domain of DNMT3A or DNMT3B, is of human origin. In some embodiments, the DNMT3 domain, such as a domain of DNMT3A or DNMT3B, is of non-human origin, such as of mouse origin.

An exemplary DNMT3A domain is set forth in SEQ ID NO:131 or 238. An exemplary DNMT3B domain is set forth in SEQ ID NO:239. In some embodiments, the effector domain comprises the sequence set forth in SEQ ID NO: 131, SEQ ID NO:238 or SEQ ID NO:239, or a portion thereof, or an amino acid sequence that has at least 90%, 91%, 92%, 93%, 94%, 95%, 96%, 97%, 98%, or 99% sequence identity to any of the foregoing.

In some embodiments, the DNMT3A domain is set forth in SEQ ID NO:131, or is a catalytically active portion thereof, or is an amino acid sequence that has at least 90%, 91%, 92%, 93%, 94%, 95%, 96%, 97%, 98%, or 99% sequence identity to SEQ ID NO:131 or the catalytically active portion thereof that exhibits DNA methyltransferase activity. In some embodiments, the DNMT3A domain is set forth in SEQ ID NO:131.

In some embodiments, the DNMT3A domain is set forth in SEQ ID NO:238, or is a catalytically active portion thereof, or is an amino acid sequence that has at least 90%, 91%, 92%, 93%, 94%, 95%, 96%, 97%, 98%, or 99% sequence identity to SEQ ID NO:238 or the catalytically active portion thereof that exhibits DNA methyltransferase activity. In some embodiments, the DNMT3A domain is set forth in SEQ ID NO:238.

In some embodiments, the effector domain is from DNMT3B or a catalytically active portion or variant thereof that exhibits DNA methyltransferase activity. An exemplary DNMT3B domain is set forth in SEQ ID NO:239, or is a catalytically active portion thereof, or is an amino acid sequence that has at least 90%, 91%, 92%, 93%, 94%, 95%, 96%, 97%, 98%, or 99% sequence identity to SEQ ID NO:239 or the catalytically active portion thereof that exhibits DNA methyltransferase activity. In some embodiments, the catalytically active portion is a contiguous portion of amino acids of SEQ ID NO:239 that includes the SAM-dependent mTase C5-type domain (e.g. corresponding to amino acids 575-853 of SEQ ID NO:239). In some embodiments, the contiguous sequence of amino acids of SEQ ID NO:239 includes at least 250 amino acids, 275 amino acids, 300 amino acids or 325 amino acids, or any value between any of the foregoing. In some embodiments, the contiguous sequence of amino acids is a contiguous portion of SEQ ID NO:239 that includes amino acids 575-853 and is from 280 amino acids to 330 amino acids in length. In some embodiments, the contiguous portion is 280 amino acids, 290 amino acids, 300 amino acids, 310 amino acids, 320 amino acids, or 330 amino acids in length, or is a length of any value between any of the foregoing.

Any of a variety of assays are known to assess or monitor methyltransferase (mTase) activity. In some embodiments, exemplary assays to assess DNA methyltransferase activity include, but are not limited to, radio DNA mTase assays, colorimetric DNA mTase activity assays, fluorescent DNA mTase activity assays, chemiluminescent/bioluminescent DNA mTase activity assays, electrochemical DNA mTase activity assays, and elctrogenerated chemiluminescence (ECL) DNA mTase activity assays. Exemplary assays are described in Poh et al. Theranostics, 2016, 6:369-391; Li et al., Methods Appl. Fluoresc., 2017, 5:012002; Deng et al., Anal Chem., 2014, 86:2117-23; and Ma et al. J Mater Chem B., 2020, 8:3488-3501.

In some embodiments, the effector domain includes at least one DNMT3L domain, or a variant thereof. The DNMT3L domain or a variant thereof may be a DNMT3L or a portion of DNMT3L, or a variant of DNMT3L or the portion thereof. DNMT3L (DNA (cytosine-5)-methyltransferase 3-like) is a catalytically inactive regulatory factor of DNA methyltransferases that can either promote or inhibit DNA methylation depending on the context. DNMT3L is essential for the function of DNMT3A and DNMT3B; DNMT3L interacts with DNMT3A and DNMT3B and significantly enhances their catalytic activity. For instance, DNMT3L interacts with the catalytic domain of DNMT3A to form a heterodimer, demonstrating that DNMT3L has dual functions of binding an unmethylated histone tail and activating DNA methyltransferase. In some embodiments, reference to a portion or variant of a DNMT3L for purposes herein refers to a sufficient C-terminal sequence portion of DNMT3L that interacts with the catalytic domain of DNMT3A or DNMT3B and is able to stimulate or promote DNA methyltransferase activity of DNMT3A or DNMT3B (see e.g. Jia et al. Nature, 2007, 449:248-251; Gowher et al. J. Biol. Chem., 2005, 280: 13341-13348). In some embodiments, the DNMT3L or portion thereof is of animal origin. In some embodiments, the domain from DNMT3L is of murine origin. In some embodiments, the domain from DNMT3L is of human origin.

In some embodiments, the DNMT3L domain is a DNMT3L, or a C-terminal portion or variant thereof, that interacts with the catalytic domain of DNMT3A to form a heterodimer to provide for a more active DNA methyltransferase. In some embodiments, the effector domain is a fusion domain of a DNMT3A domain and the DNMT3L domain (DNMT3A/3L).

In some embodiments, the DNMT3L domain is a DNMT3L, or a C-terminal portion or variant thereof, that interacts with the catalytic domain of DNMT3B to form a heterodimer to provide for a more active DNA methyltransferase. In some embodiments, the effector domain is a fusion domain of a DNMT3B domain and the DNMT3L domain (DNMT3B/3L).

In some embodiments, the DNMT3L domain is a C-terminal portion of DNMT3L composed of a contiguous C-terminal portion of the full-length DNMT3L that does not include the N-terminal cysteine-rich ATRX-Dnmt3-Dnmt3L (ADD) domain (e.g. corresponding to residues 41-73 of SEQ ID NO:133, or 75-207 of the sequence set forth in SEQ ID NO:240). In some embodiments, the DNMT3L domain is a contiguous C-terminal portion of DNMT3L that is less than 220 amino acids in length, such as between 100 and 215 amino acids, such as at or about 100, 110, 120, 130, 140, 150, 160, 170, 180, 190, 200, 210 or 215 amino acids in length, or a length between a value of any of the foregoing. In some embodiments, the DNMT3L domain is a contiguous C-terminal portion of DNMT3L that is 205, 206, 207, 208, 209, 210, 211, 212, 213, 214 or 215 amino acids in length.

An exemplary DNMT3L domain is set forth in SEQ ID NO:240, or is a portion thereof, or is an amino acid sequence that has at least 90%, 91%, 92%, 93%, 94%, 95%, 96%, 97%, 98%, or 99% sequence identity to SEQ ID NO:240 or the portion thereof. In some embodiments, the DNMT3L domain is a contiguous C-terminal portion of the full-length DNMT3L set forth in SEQ ID NO:240 that does not include the N-terminal cysteine-rich ATRX-Dnmt3-Dnmt3L (ADD) domain (corresponding to residues 75-207 of the sequence set forth in SEQ ID NO:240). In some embodiments, the DNMT3L domain is a contiguous C-terminal portion of the full-length DNMT3L set forth in SEQ ID NO:240 that is less than 220 amino acids in length, such as between 100 and 215 amino acids, such as at or about 100, 110, 120, 130, 140, 150, 160, 170, 180, 190, 200, 210 or 215 amino acids in length, or a length between a value of any of the foregoing. In some embodiments, the DNMT3L domain is a contiguous C-terminal portion of the full-length DNMT3L set forth in SEQ ID NO:240 that is 205, 206, 207, 208, 209, 210, 211, 212, 213, 214 or 215 amino acids in length.

In some embodiments, the DNMT3L domain is set forth in SEQ ID NO:241, or is a portion thereof, or is an amino acid sequence that has at least 90%, 91%, 92%, 93%, 94%, 95%, 96%, 97%, 98%, or 99% sequence identity to SEQ ID NO:241. In some embodiments, the DNMT3L domain is set forth in SEQ ID NO:241. In some embodiments, the DNMT3L domain does not contain an N-terminal methionine, such as set forth in SEQ ID NO:241.

In some embodiments, the DNMT3L domain is a human or humanized DNMT3L. Corresponding sequences of human are highly homologous to the Dnmt3L derived from mouse and have a sequence identity of at least 90% with the murine sequence. It is within the level of a skilled artisan to humanize a non-human sequence of a DNMT3L domain, such as a domain of a murine DNMT3L. In some embodiments, the effector domain includes a DNMT3L domain that is a humanized variant of the murine DMT3L set forth in SEQ ID NO:240 or a portion thereof that is able to interact with DNMT3A or DNMT3A. In some embodiments, the effector domain includes a DNMT3L domain that is a humanized variant of the murine C-terminal portion of DNMT3L set forth in SEQ ID NO:241.

An exemplary DNMT3L domain of human origin is set forth in SEQ ID NO:133, or is a portion thereof, or is an amino acid sequence that has at least 90%, 91%, 92%, 93%, 94%, 95%, 96%, 97%, 98%, or 99% sequence identity to SEQ ID NO:133 or the portion thereof. In some embodiments, the DNMT3L domain is a contiguous C-terminal portion of the full-length DNMT3L set forth in SEQ ID NO:133 that does not include the N-terminal cysteine-rich ATRX-Dnmt3-Dnmt3L (ADD) domain (corresponding to residues 41-73 of the sequence set forth in SEQ ID NO:133). In some embodiments, the DNMT3L domain is a contiguous C-terminal portion of the full-length DNMT3L set forth in SEQ ID NO:133 that is less than 220 amino acids in length, such as between 100 and 215 amino acids, such as at or about 100, 110, 120, 130, 140, 150, 160, 170, 180, 190, 200, 210 or 215 amino acids in length, or a length between a value of any of the foregoing. In some embodiments, the DNMT3L domain is a contiguous C-terminal portion of the full-length DNMT3L set forth in SEQ ID NO:133 that is 205, 206, 207, 208, 209, 210, 211, 212, 213, 214 or 215 amino acids in length.

An exemplary DNMT3L domain is set forth in SEQ ID NO:133. In some embodiments, a DNMT3L domain comprises the sequence set forth in SEQ ID NO:133, or a portion thereof, or an amino acid sequence that has at least 90%, 91%, 92%, 93%, 94%, 95%, 96%, 97%, 98%, or 99% sequence identity to SEQ ID NO:133.

In some embodiments, the DNMT3L domain comprises the sequence set forth in SEQ ID NO:242, or is a portion thereof, or is an amino acid sequence that has at least 90%, 91%, 92%, 93%, 94%, 95%, 96%, 97%, 98%, or 99% sequence identity to SEQ ID NO:242. In some embodiments, the DNMT3L domain is set forth in SEQ ID NO:242. In some embodiments, the DNMT3L domain contains an N-terminal methionine.

In some embodiments, the effector domain comprises a fusion of DNMT3A and DNMT3L (DNMT3A/L). The fusion protein contains DNMT3A and DNMT3L domains that can be any as described above. In some embodiments, the fusion protein contains the DNMT3A domain set forth in SEQ ID NO:131 and the DNMT3L domain set forth in SEQ ID NO:240, arranged in any order. In some embodiments, the fusion protein contains the DNMT3A domain set forth in SEQ ID NO:131 and the DNMT3L domain set forth in SEQ ID NO:241, arranged in any order. In some embodiments, the fusion protein contains the DNMT3A domain set forth in SEQ ID NO:131 and the DNMT3L domain set forth in SEQ ID NO:242, arranged in any order. In some embodiments, the fusion protein contains the DNMT3A domain set forth in SEQ ID NO:238 and the DNMT3L domain set forth in SEQ ID NO:240, arranged in any order. In some embodiments, the fusion protein contains the DNMT3A domain set forth in SEQ ID NO:238 and the DNMT3L domain set forth in SEQ ID NO:241, arranged in any order. In some embodiments, the fusion protein contains the DNMT3A domain set forth in SEQ ID NO:238 and the DNMT3L domain set forth in SEQ ID NO:242, arranged in any order. In some embodiments, the DNMT3A and DNMT3L domains present in a provided fusion protein are separated from each other in the fusion protein by an intervening sequence, such as the DNA-binding domain, another effector domain or a linker. In some embodiments, the domains are either directly linked to each other or they are linked via a linker, such as a peptide linker. In some embodiments, the DNMT3A and DNMT3L domains are connected as a fusion domain via a linker that connects the DNMT3A domain and the DNMT3L domain. Exemplary linkers are described herein. In some embodiments, the linker is the linker set forth in SEQ ID NO:243.

An exemplary DNMT3A/L fusion domain is set forth in SEQ ID NO:135. In some embodiments, the effector domain comprises the sequence set forth in SEQ ID NO:135, or a portion thereof, or an amino acid sequence that has at least 90%, 91%, 92%, 93%, 94%, 95%, 96%, 97%, 98%, or 99% sequence identity to SEQ ID NO:135.

An exemplary DNMT3A/L fusion domain is set forth in SEQ ID NO:137. In some embodiments, the effector domain comprises the sequence set forth in SEQ ID NO:137, or a portion thereof, or an amino acid sequence that has at least 90%, 91%, 92%, 93%, 94%, 95%, 96%, 97%, 98%, or 99% sequence identity to SEQ ID NO:137.

In some embodiments, the effector domain may comprise a LSD1 domain. LSD1 (also known as Lysine-specific histone demethylase 1A) is a histone demethylase that can demethylate lysine residues of histone H3, thereby acting as a coactivator or a corepressor, depending on the context. LSD1, including in dCas fusion proteins, has been described, for example, in WO 2013/176772, WO 2014/152432, and Kearns, N. A. et al. Nat. Methods. 12(5):401-403 (2015). An exemplary LSD1 polypeptide is set forth in SEQ ID NO:244. In some embodiments, the effector domain comprises the sequence set forth in SEQ ID NO:244, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise an EZH2 domain. EZH2 (also known as Histone-lysine N-methyltransferase EZH2) is a Catalytic subunit of the PRC2/EED-EZH2 complex, which methylates ‘Lys-9’ (H3K9me) and ‘Lys-27’ (H3K27me) of histone H3, in some aspects leading to transcriptional repression of the affected target gene. EZH2, including in dCas fusion proteins, has been described, for example, in O'Geen, H. et al., Epigenetics Chromatin. 12(1):26 (2019). An exemplary EZH2 polypeptide is set forth in SEQ ID NO:245. In some embodiments, the effector domain comprises the sequence set forth in SEQ ID NO:245, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a SunTag domain. SunTag is a repeating peptide array, which can recruit multiple copies of an antibody-fusion protein that binds the repeating peptide. The antibody-fusion protein may comprise an additional effector domain, such as a transcription repression domain (e.g. KRAB), to reduce transcription of the target gene. SunTag, including in dCas fusion proteins for gene modulation have been described, for example, in WO 2016/011070 and Tanenbaum, M. et al. Cell. 159(3):635-646 (2014). An exemplary SunTag effector domain includes a repeating GCN4 peptide having the amino acid sequence LLPKNYHLENEVARLKKLVGER (SEQ ID NO:246) separated by linkers having the amino acid sequence GGSGG (SEQ ID NO:247). In some embodiments, the effector domain comprises at least one copy of the sequence set forth in SEQ ID NO:246, or a portion thereof, or an amino acid sequence that has at least 90%, 91%, 92%, 93%, 94%, 95%, 96%, 97%, 98%, or 99% sequence identity to any of the foregoing. In some embodiments, the SunTag effector domain recruits an antibody-fusion protein that comprises a transcriptional repressor effector domain (e.g. KRAB) and binds the GCN4 peptide, thereby repressing transcription at the target site and acting as a transcriptional repressor effector domain.

### 2 Effector Domains for Transcriptional Activation

In some aspects, the DNA-targeting systems provided herein further include one or more effector domains, such as a transcriptional activator effector domain. In some embodiments, provided herein is a DNA-targeting system comprising a fusion protein comprising: (a) a DNA-binding domain capable of being targeted to a target site in a gene or regulatory DNA element thereof, such as any DNA-binding domain described above in Section I.C or Section I.D, and (b) at least one effector domain. In some aspects, the effector domain is capable of increasing transcription of the gene, such as any of the genes described in Section I.B.3. In some aspects, the effector domain comprises a transcription activation domain.

In some aspects, the effector domain activates, induces, catalyzes, or leads to increased transcription of a gene when ectopically recruited to the gene or DNA regulatory element thereof. In some embodiments, the effector domain activates, induces, catalyzes, or leads to: transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, proteolysis, ubiquitination, deubiquitination, phosphorylation, dephosphorylation, DNA methylation, DNA demethylation, histone methylation, histone demethylation, or DNA base oxidation. In some embodiments, the effector domain activates, induces, catalyzes or leads to transcription activation, transcription co-activation, or transcription elongation. In some embodiments, the effector domain induces transcription activation. In some embodiments, the effector domain has one of the aforementioned activities itself (i.e. acts directly). In some embodiments, the effector domain recruits and/or interacts with a polypeptide domain that has one of the aforementioned activities (i.e. acts indirectly).

Gene expression of endogenous mammalian genes, such as human genes, can be achieved by targeting a fusion protein comprising a DNA-binding domain, such as a dCas9, and an effector domain, such as a transcription activation domain, to mammalian genes or regulatory DNA elements thereof (e.g. a promoter or enhancer) via one or more gRNAs. Any of a variety of effector domains for transcriptional activation (e.g. transcription activation domains) are known and can be used in accord with the provided embodiments. Transcription activation domains, as well as activation of target genes by Cas fusion proteins (with a variety of Cas molecules) and the transcription activation domains, are described, for example, in WO 2014/197748, WO 2016/130600, WO 2017/180915, WO 2021/226555, WO 2021/226077, WO 2013/176772, WO 2014/152432, WO 2014/093661, Adli, M. Nat. Commun. 9, 1911 (2018), Perez-Pinera, P. et al. Nat. Methods 10, 973-976 (2013), Mali, P. et al. Nat. Biotechnol. 31, 833-838 (2013), and Maeder, M. L. et al. Nat. Methods 10, 977-979 (2013).

In some embodiments, a transcriptional activation domain comprises a domain of a protein selected from among VP64, p65, Rta, p300, CBP, VPR, VPH, HSF1, a TET protein (e.g. TET1), a partially or fully functional fragment or domain thereof, or a combination of any of the foregoing.

In some embodiments, the transcriptional activation domain comprises a VP64 domain. For example, dCas9-VP64 can be targeted to a target site by one or more gRNAs to activate a gene. VP64 is a polypeptide composed of four tandem copies of VP16, a 16 amino acid transactivation domain of the Herpes simplex virus. VP64 domains, including in dCas fusion proteins, have been described, for example, in WO 2014/197748, WO 2013/176772, WO 2014/152432, and WO 2014/093661. In some embodiments, the transcriptional activation domain comprises at least one VP16 domain, or a VP16 tetramer (“VP64”) or a variant thereof. An exemplary VP64 domain is set forth in SEQ ID NO:142. In some embodiments, the transcriptional activation domain comprises SEQ ID NO:142, or a portion thereof, or an amino acid sequence that has at least 90%, 91%, 92%, 93%, 94%, 95%, 96%, 97%, 98%, or 99% sequence identity to SEQ ID NO:142, or a portion thereof. In some embodiments, the transcriptional activation domain is set forth in SEQ ID NO:142.

In some embodiments, the transcriptional activation domain comprises a p65 activation domain (p65AD). p65AD is the principal transactivation domain of the 65 kDa polypeptide of the nuclear form of the NF-KB transcription factor. An exemplary sequence of human transcription factor p65 is available at the Uniprot database under accession number Q04206. p65 domains, including in dCas fusion proteins, have been described, for example in WO 2017/180915 and Chavez, A. et al. Nat. Methods 12, 326-328 (2015). An exemplary p65 activation domain is set forth in SEQ ID NO:248. In some embodiments, the transcriptional activation domain comprises SEQ ID NO:248, or a portion thereof, or an amino acid sequence that has at least 90%, 91%, 92%, 93%, 94%, 95%, 96%, 97%, 98%, or 99% sequence identity to SEQ ID NO:248, or a portion thereof. In some embodiments, the transcriptional activation domain is set forth in SEQ ID NO:248.

In some embodiments, the transcriptional activation domain comprises an R transactivator (Rta) domain. Rta is an immediate-early protein of Epstein-Barr virus (EBV), and is a transcriptional activator that induces lytic gene expression and triggers virus reactivation. The Rta domain, including in dCas fusion proteins, has been described, for example in WO 2017/180915 and Chavez, A. et al. Nat. Methods 12, 326-328 (2015). An exemplary Rta domain is set forth in SEQ ID NO:249. In some embodiments, the transcriptional activation domain comprises SEQ ID NO:249, or a portion thereof, or an amino acid sequence that has at least 90%, 91%, 92%, 93%, 94%, 95%, 96%, 97%, 98%, or 99% sequence identity to SEQ ID NO:249, or a portion thereof. In some embodiments, the transcriptional activation domain is set forth in SEQ ID NO:249.

In some embodiments, the transcriptional activation domain comprises a CREB-binding protein (CBP) domain or a p300 domain. In some aspects, CBP refers to the CREB-binding protein encoded by the human CREBBP gene. CBP is a coactivator that interacts with cAMP-response element binding protein (CREB). In some aspects, p300 refers to the Histone acetyltransferase p300 protein encoded by the human EP300 gene, and is a coactivator closely related to CBP. CBP and p300 each interact with a variety of transcriptional activators to affect gene transcription (Gerritsen, M. E. et al. PNAS 94(7):2927-2932 (1997)). In some embodiments, the transcriptional activation domain comprises a p300 domain. p300 domains (such as the catalytic core of p300) including in dCas fusion proteins for gene activation, has been described, for example, in WO 2016/130600, WO 2017/180915, and Hilton, I. B. et al., Nat. Biotechnol. 33(5):510-517 (2015). An exemplary human CBP sequence is set forth in SEQ ID NO:250. An exemplary human p300 sequence is set forth in SEQ ID NO:251. An exemplary p300 domain is set forth in SEQ ID NO:252. In some embodiments, the transcriptional activation domain comprises any one of SEQ ID NOS:250-252, or a portion thereof, or an amino acid sequence that has at least 90%, 91%, 92%, 93%, 94%, 95%, 96%, 97%, 98%, or 99% sequence identity to any one of SEQ ID NOS:250-252, or a portion thereof. In some embodiments, the transcriptional activation domain comprises SEQ ID NO:252, or a portion thereof, or an amino acid sequence that has at least 90%, 91%, 92%, 93%, 94%, 95%, 96%, 97%, 98%, or 99% sequence identity to SEQ ID NO:252, or a portion thereof. In some embodiments, the transcriptional activation domain is set forth in SEQ ID NO:252.

In some embodiments, the transcriptional activation domain comprises a HSF1 domain. In some aspects, HSF1 refers to the Heat shock factor protein 1 protein encoded by the human HSF1 gene. HSF1, including in dCas fusion proteins for gene activation, has been described, for example, in WO 2021/226555, WO 2015/089427, and Konermann et al. Nature 517(7536):583-8 (2015). An exemplary human HSF1 sequence is set forth in SEQ ID NO:254. An exemplary HSF1 domain sequence is set forth in SEQ ID NO:253. In some embodiments, the transcriptional activation domain comprises SEQ ID NO:253 or SEQ ID NO:254, or a portion thereof, or an amino acid sequence that has at least 90%, 91%, 92%, 93%, 94%, 95%, 96%, 97%, 98%, or 99% sequence identity to SEQ ID NO:253 or SEQ ID NO:254, or a portion thereof. In some embodiments, the transcriptional activation domain comprises SEQ ID NO:253, or a portion thereof, or an amino acid sequence that has at least 90%, 91%, 92%, 93%, 94%, 95%, 96%, 97%, 98%, or 99% sequence identity to SEQ ID NO:253, or a portion thereof. In some embodiments, the transcriptional activation domain is set forth in SEQ ID NO:253.

In some embodiments, the transcriptional activation domain comprises the tripartite activator VP64-p65-Rta (also known as VPR). VPR comprises three transcription activation domains (VP64, p65, and Rta) fused by short amino acid linkers, and can effectively upregulate target gene expression. VPR, including in dCas fusion proteins for gene activation, has been described, for example, in WO 2021/226555 and Chavez, A. et al. Nat. Methods 12, 326-328 (2015). An exemplary VPR polypeptide is set forth in SEQ ID NO:255. In some embodiments, the transcriptional activation domain comprises SEQ ID NO:255, or a portion thereof, or an amino acid sequence that has at least 90%, 91%, 92%, 93%, 94%, 95%, 96%, 97%, 98%, or 99% sequence identity to SEQ ID NO:255, or a portion thereof. In some embodiments, the transcriptional activation domain is set forth in SEQ ID NO:255.

In some embodiments, the transcriptional activation domain comprises VPH. VPH is a tripartite activator polypeptide comprising VP64, mouse p65, and HSF1. VPH, including in dCas fusion proteins for gene activation, has been described, for example, in WO 2021/226555. An exemplary VPH polypeptide is set forth in SEQ ID NO:256. In some embodiments, the transcriptional activation domain comprises SEQ ID NO:256, or a portion thereof, or an amino acid sequence that has at least 90%, 91%, 92%, 93%, 94%, 95%, 96%, 97%, 98%, or 99% sequence identity to SEQ ID NO:256, or a portion thereof. In some embodiments, the transcriptional activation domain is set forth in SEQ ID NO:256.

In some embodiments, the transcriptional activation effector domain has demethylase activity. The effector domain may include an enzyme that remove methyl (CH3-) groups from nucleic acids, proteins (in particular histones), and other molecules. The effector domain may covert the methyl group to hydroxymethylcytosine in a mechanism for demethylating DNA. Alternatively, the transcriptional activation domain can convert the methyl group to hydroxymethylcytosine in a mechanism for demethylating DNA. The effector domain can catalyze this reaction. For example, the transcriptional activation domain that catalyzes this reaction may comprise a domain from a TET protein, for example TET1 (Ten-eleven translocation methylcytosine dioxygenase 1). In some aspects, TET1 refers to the Methylcytosine dioxygenase TET1 protein encoded by the human TET1 gene. TET1 catalyzes the conversion of the modified genomic base 5-methylcytosine (5mC) into 5-hydroxymethylcytosine (5 hmC) and plays a key role in active DNA demethylation. TET1, including in dCas fusion proteins for gene activation, has been described, for example, in WO 2021/226555. An exemplary human TET1 sequence is set forth in SEQ ID NO:257. An exemplary TET1 catalytic domain is set forth in SEQ ID NO:258. In some embodiments, the transcriptional activation domain comprises SEQ ID NO:257 or SEQ ID NO:258, or a portion thereof, or an amino acid sequence that has at least 90%, 91%, 92%, 93%, 94%, 95%, 96%, 97%, 98%, or 99% sequence identity to SEQ ID NO:257 or SEQ ID NO:258, or a portion thereof. In some embodiments, the transcriptional activation domain comprises SEQ ID NO:258, or a portion thereof, or an amino acid sequence that has at least 90%, 91%, 92%, 93%, 94%, 95%, 96%, 97%, 98%, or 99% sequence identity to SEQ ID NO:258, or a portion thereof. In some embodiments, the transcriptional activation domain is set forth in SEQ ID NO:258.

In some embodiments, the effector domain may comprise a SunTag domain. SunTag is a repeating peptide array, which can recruit multiple copies of an antibody-fusion protein that binds the repeating peptide. The antibody-fusion protein may comprise an additional effector domain, such as a transcription activation domain (e.g. VP64), to induce increased transcription of the target gene. SunTag, including in dCas fusion proteins for gene activation, has been described, for example, in WO 2016/011070 and Tanenbaum, M. et al. Cell. 159(3):635-646 (2014). An exemplary SunTag effector domain includes a repeating GCN4 peptide having the amino acid sequence LLPKNYHLENEVARLKKLVGER (SEQ ID NO:246) separated by linkers having the amino acid sequence GGSGG (SEQ ID NO:247). In some embodiments, the effector domain comprises the sequence set forth in SEQ ID NO:246, a domain thereof, a portion thereof, or a variant thereof, or an amino acid sequence that has at least 90%, 91%, 92%, 93%, 94%, 95%, 96%, 97%, 98%, or 99% sequence identity to any of the foregoing. In some embodiments, the SunTag effector domain recruits an antibody-fusion protein that comprises a transcriptional activator effector domain (e.g. VP64) and binds the GCN4 peptide, thereby activating transcription at the target site and acting as a transcriptional activator effector domain.

In some aspects, provided herein are multipartite effectors for transcriptional activation, for example, multipartite transcriptional activation domains or multipartite activators. In some aspects, the multipartite activator is a fusion protein or a sequence of amino acids comprising two or more transcriptional activation domains, such as any of the transcriptional activation domains provided herein.

In some embodiments, the multipartite activator comprises two or more transcriptional activation domains, each transcriptional activation domain comprising a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2. In some aspects, the multipartite activator comprises two or more transcriptional activation domains, each transcriptional activation domain comprising a domain of a protein comprising any one of SEQ ID NOs: 407-424. In some embodiments, the multipartite activator comprises two or more transcriptional activation domains, wherein one or more of the transcriptional activation domains comprises a domain of a protein selected from among DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2. In some aspects, the transcriptional activation domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2, is or comprises any of the respective transcriptional activation domains described herein or a partially or fully functional fragment thereof, a domain thereof, or a portion thereof, such as a contiguous portion thereof of at least 30 amino acids, or a variant thereof. In some aspects, the transcriptional activation domain from DPOLA, ENL, FOXO3, HSH2D, NCOA2, NCOA3, PSA1, PYGO1, RBM39, HERC2, ZNF473, ANM2, KIBRA, IKKA, APBB1, SMN2, SERTAD2, MYBA, or NOTCH2, is or comprises any of sequences of the respective transcriptional activation domains described herein or a partially or fully functional fragment thereof, a domain thereof, or a portion thereof, such as a contiguous portion thereof of at least 30 amino acids, or a variant thereof.

In some embodiments, the multipartite activator further comprises one or more of any of the transcriptional activation domains provided herein, including any of the transcriptional activation domains described herein, such as VP64, p65, Rta, p300, CBP, VPR, VPH, HSF1, a TET protein (e.g. TET1), a partially or fully functional fragment or domain thereof, or a combination of any of the foregoing.

In some embodiments, the multipartite activator comprises any one of the SEQ ID NOS: 385-406 set forth in Table 7, or a domain, portion, or variant thereof, or an amino acid sequence that has at least 90%, 91%, 92%, 93%, 94%, 95%, 96%, 97%, 98%, or 99% sequence identity to any one of the SEQ ID NOS: 140-160 or 377 set forth in Table 7. In some embodiments, the multipartite activator is or comprises any one of the SEQ ID NOS: 140-160 or 377 set forth in Table 7. In some embodiments, the multipartite activator comprises a combination of transcriptional activation domains, such as any of the combinations of transcriptional activation domains shown in Table 7.

TABLE 7
Multipartite activators for Transcriptional Activation

| Bipartite/ | Transcriptional Activation | Exemplary amino |
|---|---|---|
| Tripartite | Domains | acid SEQ ID NO: |
| Bipartite | PYGO1, NCOA3 | 385 |
| Bipartite | NOTCH2, NCOA3 | 386 |
| Bipartite | NCOA3, NCOA3 | 387 |
| Bipartite | HSH2D, NCOA3 | 388 |
| Bipartite | FOXO3, NCOA3 | 389 |
| Bipartite | NCOA2, NCOA3 | 390 |
| Bipartite | ENL, NCOA3 | 391 |
| Bipartite | PYGO1, FOXO3 | 392 |
| Bipartite | NOTCH2, FOXO3 | 393 |
| Bipartite | NCOA3, FOXO3 | 394 |
| Bipartite | HSH2D, FOXO3 | 395 |
| Bipartite | FOXO3, FOXO3 | 396 |
| Bipartite | NCOA2, FOXO3 | 397 |
| Bipartite | ENL, FOXO3 | 398 |
| Tripartite | PYGO1, FOXO3, NCOA3 | 399 |
| Tripartite | NOTCH2, FOXO3, NCOA3 | 400 |
| Tripartite | NCOA3, FOXO3, NCOA3 | 401 |
| Tripartite | HSH2D, FOXO3, NCOA3 | 402 |
| Tripartite | FOXO3, FOXO3, NCOA3 | 403 |
| Tripartite | NCOA2, FOXO3, NCOA3 | 404 |
| Tripartite | ENL, FOXO3, NCOA3 | 405 |
| Tripartite | NCOA3, FOXO3, FOXO3 | 406 |

In some embodiments, the multipartite activator comprises any one of SEQ ID NOS:385-406, or an amino acid sequence that has at least 90%, 91%, 92%, 93%, 94%, 95%, 96%, 97%, 98%, or 99% sequence identity to any one of SEQ ID NOS:385-406. In some embodiments, the multipartite activator is set forth in any one of SEQ ID NOS:385-406, or an amino acid sequence that has at least 90%, 91%, 92%, 93%, 94%, 95%, 96%, 97%, 98%, or 99% sequence identity any one of SEQ ID NOS:385-406, or a partially or fully functional fragment thereof, a domain thereof, or a portion thereof, such as a contiguous portion thereof of at least 10, 15, 20, 25, 30, 35, 40, 45, 50, 55, 60, 65, 70, 75, or 80 amino acids, or a variant thereof. In some embodiments, the multipartite activator is set forth in any one of SEQ ID NOS:385-406.

In some embodiments, the multipartite activator comprises domains from PYGO1 and NCOA3, respectively. In some embodiments, the multipartite activator comprises SEQ ID NO:385, or an amino acid sequence that has at least 90%, 91%, 92%, 93%, 94%, 95%, 96%, 97%, 98%, or 99% sequence identity to SEQ ID NO:385. In some embodiments, the multipartite activator is set forth in SEQ ID NO:385.

In some embodiments, the multipartite activator comprises domains from NOTCH2 and NCOA3, respectively. In some embodiments, the multipartite activator comprises SEQ ID NO:386, or an amino acid sequence that has at least 90%, 91%, 92%, 93%, 94%, 95%, 96%, 97%, 98%, or 99% sequence identity to SEQ ID NO:386. In some embodiments, the multipartite activator is set forth in SEQ ID NO:386.

In some embodiments, the multipartite activator comprises domains from NCOA3 and NCOA3, respectively. In some embodiments, the multipartite activator comprises SEQ ID NO:387, or an amino acid sequence that has at least 90%, 91%, 92%, 93%, 94%, 95%, 96%, 97%, 98%, or 99% sequence identity to SEQ ID NO:387. In some embodiments, the multipartite activator is set forth in SEQ ID NO:387.

In some embodiments, the multipartite activator comprises domains from HSH2D and NCOA3, respectively. In some embodiments, the multipartite activator comprises SEQ ID NO:388, or an amino acid sequence that has at least 90%, 91%, 92%, 93%, 94%, 95%, 96%, 97%, 98%, or 99% sequence identity to SEQ ID NO:388. In some embodiments, the multipartite activator is set forth in SEQ ID NO:388.

In some embodiments, the multipartite activator comprises domains from FOXO3 and NCOA3, respectively. In some embodiments, the multipartite activator comprises SEQ ID NO:389, or an amino acid sequence that has at least 90%, 91%, 92%, 93%, 94%, 95%, 96%, 97%, 98%, or 99% sequence identity to SEQ ID NO:389. In some embodiments, the multipartite activator is set forth in SEQ ID NO:389.

In some embodiments, the multipartite activator comprises domains from NCOA2 and NCOA3, respectively. In some embodiments, the multipartite activator comprises SEQ ID NO:390, or an amino acid sequence that has at least 90%, 91%, 92%, 93%, 94%, 95%, 96%, 97%, 98%, or 99% sequence identity to SEQ ID NO:390. In some embodiments, the multipartite activator is set forth in SEQ ID NO:390.

In some embodiments, the multipartite activator comprises domains from ENL and NCOA3, respectively. In some embodiments, the multipartite activator comprises SEQ ID NO:391, or an amino acid sequence that has at least 90%, 91%, 92%, 93%, 94%, 95%, 96%, 97%, 98%, or 99% sequence identity to SEQ ID NO:391. In some embodiments, the multipartite activator is set forth in SEQ ID NO:391.

In some embodiments, the multipartite activator comprises domains from PYGO1 and FOXO3, respectively. In some embodiments, the multipartite activator comprises SEQ ID NO:392, or an amino acid sequence that has at least 90%, 91%, 92%, 93%, 94%, 95%, 96%, 97%, 98%, or 99% sequence identity to SEQ ID NO:392. In some embodiments, the multipartite activator is set forth in SEQ ID NO:392.

In some embodiments, the multipartite activator comprises domains from NOTCH2 and FOXO3, respectively. In some embodiments, the multipartite activator comprises SEQ ID NO:393, or an amino acid sequence that has at least 90%, 91%, 92%, 93%, 94%, 95%, 96%, 97%, 98%, or 99% sequence identity to SEQ ID NO:393. In some embodiments, the multipartite activator is set forth in SEQ ID NO:393.

In some embodiments, the multipartite activator comprises domains from NCOA3 and FOXO3, respectively. In some embodiments, the multipartite activator comprises SEQ ID NO:394, or an amino acid sequence that has at least 90%, 91%, 92%, 93%, 94%, 95%, 96%, 97%, 98%, or 99% sequence identity to SEQ ID NO:394. In some embodiments, the multipartite activator is set forth in SEQ ID NO:394.

In some embodiments, the multipartite activator comprises domains from HSH2D and FOXO3, respectively. In some embodiments, the multipartite activator comprises SEQ ID NO:395, or an amino acid sequence that has at least 90%, 91%, 92%, 93%, 94%, 95%, 96%, 97%, 98%, or 99% sequence identity to SEQ ID NO:395. In some embodiments, the multipartite activator is set forth in SEQ ID NO:395.

In some embodiments, the multipartite activator comprises domains from FOXO3 and FOXO3, respectively. In some embodiments, the multipartite activator comprises SEQ ID NO:396, or an amino acid sequence that has at least 90%, 91%, 92%, 93%, 94%, 95%, 96%, 97%, 98%, or 99% sequence identity to SEQ ID NO:396. In some embodiments, the multipartite activator is set forth in SEQ ID NO:396.

In some embodiments, the multipartite activator comprises domains from NCOA2 and FOXO3, respectively. In some embodiments, the multipartite activator comprises SEQ ID NO:397, or an amino acid sequence that has at least 90%, 91%, 92%, 93%, 94%, 95%, 96%, 97%, 98%, or 99% sequence identity to SEQ ID NO:397. In some embodiments, the multipartite activator is set forth in SEQ ID NO:397.

In some embodiments, the multipartite activator comprises domains from ENL and FOXO3, respectively. In some embodiments, the multipartite activator comprises SEQ ID NO:398, or an amino acid sequence that has at least 90%, 91%, 92%, 93%, 94%, 95%, 96%, 97%, 98%, or 99% sequence identity to SEQ ID NO:398. In some embodiments, the multipartite activator is set forth in SEQ ID NO:398.

In some embodiments, the multipartite activator comprises domains from PYGO1, FOXO3, and NCOA3, respectively. In some embodiments, the multipartite activator comprises SEQ ID NO:399, or an amino acid sequence that has at least 90%, 91%, 92%, 93%, 94%, 95%, 96%, 97%, 98%, or 99% sequence identity to SEQ ID NO:399. In some embodiments, the multipartite activator is set forth in SEQ ID NO:399.

In some embodiments, the multipartite activator comprises domains from NOTCH2, FOXO3, and NCOA3, respectively. In some embodiments, the multipartite activator comprises SEQ ID NO:400, or an amino acid sequence that has at least 90%, 91%, 92%, 93%, 94%, 95%, 96%, 97%, 98%, or 99% sequence identity to SEQ ID NO:400. In some embodiments, the multipartite activator is set forth in SEQ ID NO:400.

In some embodiments, the multipartite activator comprises domains from NCOA3, FOXO3, and NCOA3, respectively. In some embodiments, the multipartite activator comprises SEQ ID NO:401, or an amino acid sequence that has at least 90%, 91%, 92%, 93%, 94%, 95%, 96%, 97%, 98%, or 99% sequence identity to SEQ ID NO:401. In some embodiments, the multipartite activator is set forth in SEQ ID NO:401.

In some embodiments, the multipartite activator comprises domains from HSH2D, FOXO3, and NCOA3, respectively. In some embodiments, the multipartite activator comprises SEQ ID NO:402, or an amino acid sequence that has at least 90%, 91%, 92%, 93%, 94%, 95%, 96%, 97%, 98%, or 99% sequence identity to SEQ ID NO:402. In some embodiments, the multipartite activator is set forth in SEQ ID NO:402.

In some embodiments, the multipartite activator comprises domains from FOXO3, FOXO3, and NCOA3, respectively. In some embodiments, the multipartite activator comprises SEQ ID NO:403, or an amino acid sequence that has at least 90%, 91%, 92%, 93%, 94%, 95%, 96%, 97%, 98%, or 99% sequence identity to SEQ ID NO:403. In some embodiments, the multipartite activator is set forth in SEQ ID NO:403.

In some embodiments, the multipartite activator comprises domains from NCOA2, FOXO3, and NCOA3, respectively. In some embodiments, the multipartite activator comprises SEQ ID NO:404, or an amino acid sequence that has at least 90%, 91%, 92%, 93%, 94%, 95%, 96%, 97%, 98%, or 99% sequence identity to SEQ ID NO:404. In some embodiments, the multipartite activator is set forth in SEQ ID NO:404.

In some embodiments, the multipartite activator comprises domains from ENL, FOXO3, and NCOA3, respectively. In some embodiments, the multipartite activator comprises SEQ ID NO:405, or an amino acid sequence that has at least 90%, 91%, 92%, 93%, 94%, 95%, 96%, 97%, 98%, or 99% sequence identity to SEQ ID NO:405. In some embodiments, the multipartite activator is set forth in SEQ ID NO:405.

In some embodiments, the multipartite activator comprises domains from NCOA3, FOXO3, and FOX03, respectively. In some embodiments, the multipartite activator comprises SEQ ID NO:406, or an amino acid sequence that has at least 90%, 91%, 92%, 93%, 94%, 95%, 96%, 97%, 98%, or 99% sequence identity to SEQ ID NO:406. In some embodiments, the multipartite activator is set forth in SEQ ID NO:406.

### F. Fusion Proteins

In some aspects, the DNA-targeting systems provided herein include fusion proteins. In some embodiments, the fusion protein comprises: (a) a DNA-binding domain capable of being targeted to a target site for one or more genes, and (b) at least one transcriptional repressor effector domain for repressing transcription of the one or more genes. In some embodiments, the fusion protein comprises: (a) a DNA-binding domain capable of being targeted to a target site for one or more genes, and (b) at least one transcriptional activator effector domain for increasing transcription of the one or more genes.

In some aspects, the fusion protein comprises at least one of any of the DNA-binding domains described herein in Section I.C or Section I.D, and at least one of any of the effector domains described herein. In some embodiments, the fusion protein contains a CRISPR/Cas-based DNA-binding domain, such described in Section I.C., and at least one effector domain for transcriptional repression, such as described in section I.E.1. In some aspects, the fusion protein is targeted to a target site in a gene or regulatory element thereof, and leads to reduced or repressed transcription of the gene. In some aspects, the fusion protein is targeted to target sites in a combination of genes or regulatory elements thereof, and leads to reduced or repressed transcription of each of the genes.

In some embodiments, the fusion protein comprises at least one of any of the DNA-binding domains described herein in Section I.C or Section I.D, and at least one of any of the effector domains described herein. In some embodiments, the fusion protein contains a CRISPR/Cas-based DNA-binding domain, such as described in Section I.C., and at least one effector domain for transcriptional activation, as described in section I.E.2. In some aspects, the fusion protein is targeted to a target site in a gene or regulatory element thereof, and leads to increased or activated transcription of the gene. In some aspects, the fusion protein is targeted to target sites in a combination of genes or regulatory elements thereof, and leads to increased or activated transcription of each of the genes.

In some embodiments, the DNA-binding domain and effector domain of the fusion protein are heterologous, i.e. the domains are from different species, or at least one of the domains is not found in nature. In some aspects, the fusion protein is an engineered fusion protein, i.e. the fusion protein is not found in nature.

In some embodiments, the at least one effector domain is fused to the N-terminus, the C-terminus, or both the N-terminus and the C-terminus, of the DNA-binding domain or a component thereof. The at least one effector domain may be fused to the DNA-binding domain directly, or via any intervening amino acid sequence, such as a linker sequence or a nuclear localization sequence (NLS).

In some embodiments, the fusion protein of a provided DNA-binding system, or a DNA-targeting module thereof, comprises, from N- to C-terminal order: a transcriptional repressor effector domain and a DNA-binding domain. In some embodiments, the fusion protein of a provided DNA-binding system, or a DNA-targeting module thereof, comprises, from N- to C-terminal order: a DNA-binding domain and a transcriptional repressor effector domain.

In some embodiments, the fusion protein of a provided DNA-binding system, or a DNA-targeting module thereof, comprises, from N- to C-terminal order: a transcriptional activator effector domain and a DNA-binding domain. In some embodiments, the fusion protein of a provided DNA-binding system, or a DNA-targeting module thereof, comprises, from N- to C-terminal order: a DNA-binding domain and a transcriptional activator effector domain.

In some embodiments, the at least one effector domain of the fusion protein includes more than one effector domain. In some embodiments, the fusion protein includes 2, 3 or 4 effector domains, or more than 4 effector domains. In some embodiments, at least two of the effector domains of the fusion protein are different. In some embodiments, each of the effector domains of the fusion protein are different. In some embodiments, the at least one effector domain includes two effector domains in which the two effector domains are different. In some embodiments, the effector domains and the DNA-binding domain can be arranged in any order.

In some embodiments, each of the effector domains is a transcriptional repressor effector domain. In some aspects, each of the effector domains is a transcriptional activator effector domain.

In some embodiments, the at least one effector domain of the fusion protein includes two different effector domains. The two different effector domains and the DNA-binding domain can be arranged in any order. In some embodiments, each of the effector domains are N-terminal to the DNA-binding domain in which a first effector domain is fused to the N-terminus of the second effector domain and the second effector domain is fused to the N-terminus of the DNA-binding domain. In some embodiments, the fusion protein of a provided DNA-binding system, or a DNA-targeting module thereof, comprises from N- to C-terminal order: a first effector domain, a second effector domain and the DNA binding domain. In some embodiments, each of the effector domains are C-terminal to the DNA-binding domain in which a first effector domain is fused to the C-terminus of the DNA-binding domain and the second effector domain is fused to the C-terminus of the first effector domain. In some embodiments, the fusion protein of a provided DNA-binding system, or a DNA-targeting module thereof, comprises from N- to C-terminal order: a DNA-binding domain, a first effector domain, and a second effector domain. In some embodiments, the DNA-binding domain is between the effector domains, in which one effector domain is fused to the N-terminus of the DNA-binding domain and the other effector domain is fused to the C-terminus of the DNA-binding domain. In some embodiments, the fusion protein of a provided DNA-binding system, or a DNA-targeting module thereof, comprises from N- to C-terminal order: a first effector domain, a DNA-binding domain, and a second effector domain. In some embodiments, one or more of the components may be fused to each other directly, or via any intervening amino acid sequence, such as via a linker sequence or a nuclear localization sequence (NLS).

In some embodiments, the fusion protein comprises one or more linkers. In some embodiments, the linker is a peptide linker. In some embodiments, the one or more linkers connect the DNA-binding domain or a component thereof to the at least one effector domain. A linker may be included anywhere in the polypeptide sequence of the fusion protein, for example, between the effector domain and the DNA-binding domain or a component thereof. A linker may be of any length and designed to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A skilled artisan can readily choose an appropriate linker for the connection of two domains. In some embodiments, the linker is a flexible linker. Flexible linkers are generally composed of small, non-polar or polar residues such as glycine, serine or threonine. A linker may include sequential or tandem repeats of an amino acid sequence that is 2 to 20 amino acids in length. Linkers may be rich in amino acids glycine (G), serine (S), and/or alanine (A). Linkers may include, for example, a GS linker. An exemplary GS linker is represented by the sequence GGGGS (SEQ ID NO:259). A linker may comprise repeats of a sequence, for example as represented by the formula (GGGGS)n, wherein n is an integer that represents the number of times the GGGGS sequence is repeated (e.g. between 1 and 10 times). The number of times a linker sequence is repeated can be adjusted to optimize the linker length and achieve appropriate separation of the functional domains. For example, in some embodiments, the linker is the (GGGGS)ₙ linker, whereby n is an integer of 1 to 10. Other examples of linkers may include, for example, GGGGG (SEQ ID NO:260), GGAGG (SEQ ID NO:261), GGGGSSS (SEQ ID NO:262), or GGGGAAA (SEQ ID NO:263).

In some embodiments, artificial linker sequences can be used. In some embodiments, the linker is EASGSGRASPGIPGSTR (SEQ ID NO:264). In some embodiments, the linker is linker is GIHGVPAA (SEQ ID NO:265). In some embodiments, the linker is SSGNSNANSRGPSFSSGLVPLSLRGSH (SEQ ID NO:243). In some embodiments, the linker is KRPAATKKAGQAKKKKASDAKSLTAWS (SEQ ID NO:266).

In some embodiments, inclusion of a linker in the fusion protein leads to enhanced modulation (such as repression or activation) of the target gene.

In some embodiments, the linker is an XTEN linker. In some aspects, an XTEN linker is a recombinant polypeptide (e.g., an unstructured recombinant peptide) lacking hydrophobic amino acid residues. Exemplary XTEN linkers are described in, for example, Schellenberger et al., Nature Biotechnology 27, 1186-1190 (2009) or WO 2021/247570. In some embodiments, inclusion of a linker in the fusion protein leads to enhanced repression of the target gene. In some embodiments, a linker comprises the sequence set forth in SEQ ID NO:267, or a portion thereof, or an amino acid sequence that has at least 90%, 91%, 92%, 93%, 94%, 95%, 96%, 97%, 98%, or 99% sequence identity to SEQ ID NO:267. In some aspects, the linker comprises the sequence set forth in SEQ ID NO:233, or a contiguous portion of SEQ ID NO:267 of at least 5, 10, 15, 20, 25, 30, 35, 40, 45, 50, 55, 60, 65, 70 or 75 amino acids. In some aspects, the linker consists of the sequence set forth in SEQ ID NO:267, or a contiguous portion of SEQ ID NO:267 of at least 5, 10, 15, 20, 25, 30, 35, 40, 45, 50, 55, 60, 65, 70 or 75 amino acids. In some embodiments, the linker comprises the sequence set forth in SEQ ID NO:267. In some embodiments, the linker consists of the sequence set forth in SEQ ID NO:267. In some embodiments, a linker comprises the sequence set forth in SEQ ID NO:268, or a portion thereof, or an amino acid sequence that has at least 90%, 91%, 92%, 93%, 94%, 95%, 96%, 97%, 98%, or 99% sequence identity to any of the foregoing. In some aspects, the linker comprises the sequence set forth in SEQ ID NO:268, or a contiguous portion of SEQ ID NO:268 of at least 5, 10, or15 amino acids. In some aspects, the linker consists of the sequence set forth in SEQ ID NO:268, or a contiguous portion of SEQ ID NO:268 of at least 5, 10 or 15 amino acids. In some embodiments, the linker comprises the sequence set forth in SEQ ID NO:268. In some embodiments, the linker consists of the sequence set forth in SEQ ID NO:268. Appropriate linkers may be selected or designed based rational criteria known in the art, for example as described in Chen et al. Adv. Drug Deliv. Rev. 65(10):1357-1369 (2013). In some embodiments, a linker comprises a linker described in WO 2021/247570.

In some embodiments, the fusion protein of the DNA-targeting system, or a DNA-targeting module thereof, comprises one or more nuclear localization signals (NLS). In some embodiments, a fusion protein described herein comprises one or more nuclear localization sequences (NLSs), such as about or more than about 1, 2, 3, 4, 5, 6, 7, 8, 9, 10, or more NLSs. When more than one NLS is present, each may be selected independently of the others, such that a single NLS may be present in more than one copy and/or in combination with one or more other NLSs present in one or more copies. Non-limiting examples of NLSs include an NLS sequence derived from: the NLS of the SV40 virus large T-antigen, having the amino acid sequence PKKKRKV (SEQ ID NO:269); the NLS from nucleoplasmin (e.g. the nucleoplasmin bipartite NLS with the sequence KRPAATKKAGQAKKKK (SEQ ID NO:270)); the c-myc NLS having the amino acid sequence PAAKRVKLD (SEQ ID NO:271) or RQRRNELKRSP (SEQ ID NO:272); the hRNPA1 M9 NLS having the sequence NQSSNFGPMKGGNFGGRSSGPYGGGGQYFAKPRNQGGY (SEQ ID NO:273); the sequence RMRIZFKNKGKDTAELRRRRVEVSVELRKAKKDEQILKRRNV (SEQ ID NO:274) of the 1BB domain from importin-alpha; the sequences VSRKRPRP (SEQ ID NO:275) and PPKKARED (SEQ ID NO:276) of the myoma T protein; the sequence PQPKKKPL (SEQ ID NO:277) of human p53; the sequence SALIKKKKKMAP (SEQ ID NO:278) of mouse c-abl IV; the sequences DRLRR (SEQ ID NO:279) and PKQKKRK (SEQ ID NO:280) of the influenza virus NS1; the sequence RKLKKKIKKL (SEQ ID NO:281) of the Hepatitis virus delta antigen; the sequence REKKKFLKRR (SEQ ID NO:282) of the mouse Mxl protein; the sequence KRKGDEVDGVDEVAKKKSKK (SEQ ID NO:283) of the human poly(ADP-ribose) polymerase; and the sequence RKCLQAGMNLEARKTKK (SEQ ID NO:284) of the steroid hormone receptors (human) glucocorticoid. The NLS may comprise a portion of any of the foregoing. In general, the one or more NLSs are of sufficient strength to drive accumulation of the fusion protein in a detectable amount in the nucleus of a eukaryotic cell. In general, strength of nuclear localization activity may derive from the number of NLSs in the fusion protein, the particular NLS(s) used, or a combination of these factors. Detection of accumulation in the nucleus may be performed by any suitable technique. For example, a detectable marker may be fused to the fusion protein, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the fusion protein (e.g. an assay for altered gene expression activity in a cell transformed with the DNA-targeting system comprising the fusion protein), as compared to a control condition (e.g. an untransformed cell).

In some embodiments, the NLS is linked to the N-terminus or the C-terminus of the DNA-binding domain via a linker. In some embodiments, the NLS is linked to the N-terminus or the C-terminus of an effector domain via a linker. The linker may be any linker as described above. In some embodiemnts, the linker is GIHGVPAA (SEQ ID NO:265). In some embodiments, the NLS and linker has the sequence PKKKRKVGIHGVPAA (SEQ ID NO:285).

In some configurations, the N- or C-terminus of the fusion protein can be linked to a moiety for detection and/or purification. In some aspects, the moiety is or includes a Flag tag DYKDDDDK (SEQ ID NO:286), a 3×Flag tag MDYKDHDGDYKDHDIDYKDDDDK (SEQ ID NO:287), an HA tag YPYDVPDYA (SEQ ID NO:288) or a His tag, such as HHHHHH (SEQ ID NO:289).

In some embodiments, the fusion protein is a dCas-KRAB fusion protein, such as dSpCas9-KRAB. In some embodiments, the fusion protein is dSpCas9-KRAB. In some embodiments, the fusion protein comprises the sequence set forth in SEQ ID NO:70, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:70. In some embodiments, the fusion protein is encoded by the nucleotide sequence set forth in SEQ ID NO:71.

In some embodiments, the fusion protein is a dCas-KRAB-DNMT3A/3L fusion protein, such as dSpCas9-KRAB-DNMT3A/3L. In some embodiments, the fusion protein is dSpCas9-KRAB-DNMT3A/3L. In some embodiments, the fusion protein comprises the sequence set forth in SEQ ID NO:75, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:75. In some embodiments, the fusion protein is encoded by the nucleotide sequence set forth in SEQ ID NO:74.

In some embodiments, the fusion protein is a dCas-VP64 fusion protein, such as dSpCas9-2xVP64, which is a fusion of dSpCas9 fused to two copies of VP64. In some embodiments, the fusion protein is dSpCas9-2xVP64. In some embodiments, the fusion protein comprises the sequence set forth in SEQ ID NO:77, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:77. In some embodiments, the fusion protein is encoded by the nucleotide sequence set forth in SEQ ID NO:76.

In some embodiments, the fusion protein is a dCas-KRAB-DNMT3A/3L fusion protein comprising an XTEN linker. In some embodiments, the fusion protein comprises the sequence set forth in SEQ ID NO:140 or 141, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:140. In some embodiments, the fusion protein comprises the sequence set forth in SEQ ID NO:141.

In some embodiments, the fusion protein comprises from N-terminus to C-terminus: (i) a DNA-binding domain, and (ii) a KRAB domain or EZH2 domain. In some embodiments, the fusion protein comprises from N-terminus to C-terminus: (i) a DNMT3A domain, (ii) a DNMT3L domain, (iii) a DNA-binding domain, and (iv) a KRAB domain or EZH2 domain. In some embodiments, the fusion protein comprises from N-terminus to C-terminus: (i) a DNMT3A domain, (ii) a DNMT3L domain, (iii) a KRAB domain or EZH2 domain, and (iv) a DNA-binding domain. In some embodiments, the fusion protein comprises from N-terminus to C-terminus: (i) a DNMT3B domain, (ii) a DNMT3L domain, (iii) a DNA-binding domain, and (iv) a KRAB domain or EZH2 domain. In some embodiments, the fusion protein comprises: a DNA-binding domain, a DNMT3B domain, and a DNMT3L domain. In some embodiments, the fusion protein comprises: a DNA-binding domain, a DNMT3B domain, a DNMT3L domain, and a KRAB domain or EZH2 domain. In some embodiments, the DNA-binding domain is any suitable DNA-binding domain, including any DNA-binding domain described herein. In some embodiments, the DNA-binding domain is a dCas protein, such as dSpCas9.

In some embodiments, the DNMT3A domain comprises the sequence set forth in SEQ ID NO:131 or 238, or a sequence having at least 90% identity thereto. In some embodiments, the DNMT3L domain comprises the sequence set forth in any one of SEQ ID NOS: 133 and 240-242, or a sequence having at least 90% identity thereto. In some embodiments, the DNMT3B domain comprises the sequence set forth in SEQ ID NO:239 or 360, or a sequence having at least 90% identity thereto. In some embodiments, the fusion protein comprises a DNMT3A-DNMT3L fusion domain. In some embodiments, the DNMT3A-DNMT3L fusion domain comprises the sequence set forth in SEQ ID NO:135 or 137, or a sequence having at least 90% identity thereto. In some embodiments the fusion protein comprises a DNMT3B-DNMT3L fusion domain. In some embodiments, the DNMT3B-DNMT3L fusion domain comprises the sequence set forth in SEQ ID NO:363, or a sequence having at least 90% identity thereto. In some embodiments, the KRAB domain is selected from: a KRAB domain from KOX1, a KRAB domain from ZIM3, and a KRAB domain from ZNF324. In some embodiments, the KRAB domain comprises the sequence set forth in any one of SEQ ID NOS:70, 235, and 355-358, or a sequence having at least 90% identity thereto. In some embodiments, the EZH2 domain comprises the sequence set forth in SEQ ID NO:359, or a sequence having at least 90% identity thereto.

In some embodiments, the fusion protein further comprises an NLS, such as any NLS provided herein. In some embodiments, the NLS is a nucleoplasmin NLS or an SV40 NLS. In some embodiments, the NLS comprises the sequence set forth in SEQ ID NO:269 or 270, or a sequence having at least 90% identity thereto.

In some embodiments, the fusion protein further comprises a linker, such as any suitable linker provided herein. In some embodiments, the linker comprises the sequence set forth in any one of SEQ ID NOS:243, 361 or 362, or a sequence having at least 90% identity thereto.

In some embodiments, the fusion protein comprises the sequence set forth in any one of SEQ ID NOS:332-351, a portion thereof, or a sequence having at least 90% sequence identity to any of the foregoing. In some embodiments, the fusion protein comprises the sequence set forth in any one of SEQ ID NOS:365-384, or a sequence having at least 90% sequence identity to any of the foregoing. In some embodiments, the fusion protein comprises the sequence set forth in any one of SEQ ID NOS:365-384.

### 1. Split Fusion Proteins

In some embodiments, the fusion protein is a split protein, i.e. comprises two or more separate polypeptide domains that interact or self-assemble to form a functional fusion protein. In some aspects, the split fusion protein comprises a dCas9 and an effector domain. In some aspects, the fusion protein comprises a split dCas9-effector domain fusion protein.

In some embodiments, the split fusion protein is assembled from separate polypeptide domains comprising trans-splicing inteins. Inteins are internal protein elements that self-excise from their host protein and catalyze ligation of flanking sequences with a peptide bond. In some embodiments, the split fusion protein is assembled from a first polypeptide comprising an N-terminal intein and a second polypeptide comprising a C-terminal intein. In an exemplary embodiment, the N terminal intein is the N terminal Npu Intein set forth in SEQ ID NO:290. In some embodiments, the C terminal intein is the C terminal Npu intein set forth in SEQ ID NO:291.

In some embodiments, the split fusion protein comprises a split dCas9-effector domain fusion protein assembled from two polypeptides. In an exemplary embodiment, the first polypeptide comprises an effector domain catalytic domain and an N-terminal fragment of dSpCas9, followed by an N terminal Npu Intein (effector domain-dSpCas9-573N), and the second polypeptide comprises a C terminal Npu Intein, followed by a C-terminal fragment of dSpCas9 (dSpCas9-573C). The N- and C-terminal fragments of the fusion protein are split at position 573Glu of the SpCas9 molecule, with reference to SEQ ID NO:126 (corresponding to residue 572Glu of the dSpCas9 molecule set forth in SEQ ID NO:127). In some aspects, the N-terminal Npu Intein (SEQ ID NO:290) and C-terminal Npu Intein (set forth in SEQ ID NO:291) may self-excise and ligate the two fragments, thereby forming the full-length dSpCas9-effector domain fusion protein when expressed in a cell.

In some embodiments, the polypeptides of a split protein may interact non-covalently to form a complex that recapitulates the activity of the non-split protein. For example, two domains of a Cas enzyme expressed as separate polypeptides may be recruited by a gRNA to form a ternary complex that recapitulates the activity of the full-length Cas enzyme in complex with the gRNA, for example as described in Wright et al. PNAS 112(10):2984-2989 (2015). In some embodiments, assembly of the split protein is inducible (e.g. light inducible, chemically inducible, small-molecule inducible).

In some aspects, the two polypeptides of a split fusion protein may be delivered and/or expressed from separate vectors, such as any of the vectors described herein. In some embodiments, the two polypeptides of a split fusion protein may be delivered to a cell and/or expressed from two separate AAV vectors, i.e. using a split AAV-based approach, for example as described in WO 2017/197238.

Approaches for the rationale design of split proteins and their delivery, including Cas proteins and fusions thereof, are described, for example, in WO 2016/114972, WO 2017/197238, Zetsche. et al. Nat. Biotechnol. 33(2):139-42 (2015), Wright et al. PNAS 112(10):2984-2989 (2015), Truong. et al. Nucleic Acids Res. 43, 6450-6458 (2015), and Fine et al. Sci. Rep. 5, 10777 (2015).

### II. POLYNUCLEOTIDES, VECTORS, AND RELATED METHODS FOR DELIVERY

In some aspects, provided are polynucleotides encoding any of the DNA-targeting systems described herein in Section I or a portion or a component of any of the foregoing. In some aspects, the polynucleotides can encode any of the components of the DNA-targeting systems, and/or any nucleic acid or proteinaceous molecule necessary to carry out aspects of the methods of the disclosure. In particular embodiments, provided are polynucleotides encoding any of the fusion proteins described herein, for example in Section I.F. Also provided herein are polynucleotides encoding any of the gRNAs described herein, for example in Section I.C.ii.

In some embodiments, provided are polynucleotides comprising the gRNAs described herein. In some embodiments, the gRNA is transcribed from a genetic construct (i.e. vector or plasmid) in the target cell. In some embodiments, the gRNA is produced by in vitro transcription and delivered to the target cell. In some embodiments, the gRNA comprises one or more modified nucleotides for increased stability. In some embodiments, the gRNA is delivered to the target cell pre-complexed as a RNP with the fusion protein.

In some embodiments, a provided polynucleotide encodes a fusion protein as described herein that includes (a) a DNA-binding domain capable of being targeted to a target site of a target gene as described; and (b) at least one effector domain capable of modulating (such as increasing or decreasing) transcription of the gene. In some embodiments, the fusion protein includes a fusion protein of a Cas protein or variant thereof and at least one effector domain capable of modulating (such as increasing or decreasing) transcription of a gene. In particular example, the Cas is a dCas, such as dCas9. In some embodiments, the dCas9 is a dSpCas9, such as the polynucleotide encoding a dSpCas9 set forth in SEQ ID NO:127. Examples of such domains and fusion proteins include any as described in Section I.

In some embodiments, the polynucleotide encodes a dCas-VP64 fusion protein, such as dSpCas9-2xVP64. In some embodiments, the polynucleotide comprises the sequence set forth in SEQ ID NO:76, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:76. In some embodiments, the polynucleotide encodes an amino acid sequence comprising SEQ ID NO:77, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:77.

In some embodiments, the polynucleotide encodes a dCas-KRAB fusion protein, such as dSpCas9-KRAB. In some embodiments, the polynucleotide encodes an amino acid sequence comprising SEQ ID NO:138, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:138.

In some embodiments, the polynucleotide encodes a dCas-KRAB-DNMT3A/3L fusion protein, such as dSpCas9-KRAB-DNMT3A/3L. In some embodiments, the polynucleotide encodes an amino acid sequence comprising SEQ ID NO:139, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:139.

In some embodiments, the polynucleotide encodes a dCas-KRAB-DNMT3A/3L fusion protein, such as dSpCas9-KRAB-DNMT3A/3L. In some embodiments, the polynucleotide encodes an amino acid sequence comprising SEQ ID NO:140, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:140.

In some embodiments, the polynucleotide encodes a dCas-KRAB-DNMT3A/3L fusion protein, such as dSpCas9-KRAB-DNMT3A/3L. In some embodiments, the polynucleotide encodes an amino acid sequence comprising SEQ ID NO:141, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:141.

In some embodiments, the polynucleotide is RNA or DNA. In some embodiments, the polynucleotide, such as a polynucleotide encoding a provided fusion protein, is mRNA. The mRNA can be 5′ capped and/or 3′ polyadenylated. In another embodiment, a polynucleotide provided herein, such as a polynucleotide encoding a provided fusion protein, is DNA. The DNA can be present in a vector.

Also provided herein is a vector that contains any of the provided polynucleotides. In some embodiments, the vector comprises a genetic construct, such as a plasmid or an expression vector.

In some embodiments, the expression vector comprising the sequence encoding the fusion protein of a DNA-targeting system provided herein can further comprise a polynucleotide sequence encoding at least one gRNA. The sequence encoding the gRNA can be operably linked to at least one transcriptional control sequence for expression of the gRNA in the cell. For example, DNA encoding the gRNA can be operably linked to a promoter sequence that is recognized by RNA polymerase III (Pol III). Examples of suitable Pol III promoters include, but are not limited to, mammalian U6, U3, H1, and 7SL RNA promoters.

In some embodiments, provided is a vector containing a polynucleotide that encodes a fusion protein comprising a DNA-binding domain comprising a dCas and at least one effector domain capable of modulating (e.g. increasing or decreasing) transcription of a gene, and a polynucleotide(s) encoding at least one gRNA. In some embodiments, the dCas is a dCas9, such as dSpCas9. In some embodiments, the polynucleotide encodes a fusion protein that includes a dSpCas9 set forth in SEQ ID NO:127. In some embodiments, the polynucleotide encoding at least one gRNA encodes a gRNA as described in Section I.C.2. For example, the polynucleotide can encode a gRNA comprising a spacer sequence selected from any one of SEQ ID NOS:35-40, 44-67, 91-101, 113-123, 212-223, 296-299, 303-305, and 309-311, or a contiguous portion thereof of at least 14 nt. In another example, the polynucleotide can encode a gRNA comprising a spacer sequence selected from any one of SEQ ID NOS:41-43, 79, 157-169, 171, 178-183, and 192-199, or a contiguous portion thereof of at least 14 nt.

In some embodiments, the polynucleotide encodes the fusion protein and the at least one gRNA.

In some embodiments, the polynucleotide as provided herein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available.

In some embodiments, a polynucleotide described herein can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EFI), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with the DNA-targeting systems, various promoters such as RNA polymerase III promoters, including for example U6 and H1, can be advantageous. Descriptions of and parameters for enhancing the use of such promoters are known in the art, and additional information and approaches are regularly being described; see, e.g., Ma, H. et al., Molecular Therapy-Nucleic Acids 3, e161 (2014) doi:10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter (e.g. a T cell specific promoter), etc.).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXTI, pSG5, pSVK3, pBPV, pMSG, and pSVLSV40 (Pharmacia). Other vectors can be used so long as they are compatible with the host cell.

In some embodiments, the vector is a viral vector, such as an adeno-associated virus (AAV) vector, a retroviral vector, a lentiviral vector, or a gammaretroviral vector. In some embodiments In some embodiments, the viral vector is an adeno-associated virus (AAV) vector. In some embodiments, the AAV vector is selected from among an AAV1, AAV2, AAV3, AAV4, AAV5, AAV6, AAV7, AAV8, or AAV9 vector. In some embodiments, the vector is a lentiviral vector. In some embodiments, the vector is a non-viral vector, for example a lipid nanoparticle, a liposome, an exosome, or a cell penetrating peptide. In some embodiments, the vector comprises one vector, or two or more vectors.

In some embodiments, a vector described herein is or comprises a lipid nanoparticle (LNP). Among provided embodiments, is a lipid nanoparticle that contains any of the provided polynucleotides for delivery of an epigenetic-modifying DNA-targeting system. In some embodiments, the LNP contains a polynucleotide that encodes a fusion protein as provided herein that includes (a) a DNA-binding domain capable of being targeted to a target site for one or more genes; and (b) at least one effector domain. In some embodiments, the DNA-binding domain is a Cas (e.g. dCas) and the LNP further includes a gRNA. In some embodiments, the polynucleotide encoding the fusion protein is an mRNA and the gRNA is provided as an RNA.

In some embodiments, any of the epigenetic-modifying DNA-targeting systems, gRNAs, Cas-gRNA combinations, polynucleotides, fusion proteins, or components thereof described herein, are incorporated in lipid nanoparticles (LNPs), such as for delivery. In some embodiments, the lipid nanoparticle is a vector for delivery. In some embodiments, the nanoparticle may comprise at least one lipid. The lipid may be selected from, but is not limited to, dLin-DMA, dLin-K-DMA, 98N12-5, C12-200, dLin-MC3-DMA, dLin-KC2-DMA, DODMA, PLGA, PEG, PEG-DMG and PEGylated lipids. In another aspect, the lipid may be a cationic lipid such as, but not limited to, dLin-DMA, dLin-D-DMA, dLin-MC 3-DMA, dLin-KC2-DMA and DODMA. Typically, the LNPs are composed of two or more lipids, such as 3, 4 or 5 lipids. In some embodiments, at least lipid is either ionoizable cationic or cationic.

Lipid nanoparticles can be used for the delivery of encapsulated or associated (e.g., complexed) therapeutic agents, including nucleic acids and proteins, such as those encoding and/or comprising CRISPR/Cas systems. See, e.g., U.S. Pat. Nos. 10,723,692, 10,941,395, and WO 2015/035136.

In some embodiments, the provided methods involve use of a lipid nanoparticle (LNP) comprising mRNA, such as mRNA encoding a protein component of any of the provided DNA-targeting systems, for example any of the fusion proteins provided herein. In some embodiments, the mRNA can be produced using methods known in the art such as in vitro transcription. In some embodiments of the method, the mRNA comprises a 5′ cap. In some embodiments, the 5′ cap is an altered nucleotide on the 5′ end of primary transcripts such as messenger RNA. In some aspects, the 5′ caps of the mRNA improves one or more of RNA stability and processing, mRNA metabolism, the processing and maturation of an RNA transcript in the nucleus, transport of mRNA from the nucleus to the cytoplasm, mRNA stability, and efficient translation of mRNA to protein. In some embodiments, a 5′ cap can be a naturally-occurring 5′ cap or one that differs from a naturally-occurring cap of an mRNA. A 5′ cap may be any 5′ cap known to a skilled artisan. In certain embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof. For instance, the 5′ cap may include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded protein and/or stability of the mRNA itself. Such elements can include, for example, posttranslational regulatory elements such as a woodchuck hepatitis virus post-transcriptional regulatory element (WPRE). In some embodiments, the mRNA comprises at least one nucleoside modification. The mRNA may contain modifications of naturally-occurring nucleosides to nucleoside analogs. Any nucleoside analogs known in the art are envisioned. Such nucleoside analogs can include, for example, those described in U.S. Pat. No. 8,278,036. In certain embodiments of the method, the nucleoside modification is selected from the group consisting of a modification from uridine to pseudouridine and uridine to N1-methyl pseudouridine. In particular embodiments of the method the nucleoside modification is from uridine to pseudouridine.

In some embodiments, LNPs useful for in the present methods comprise a cationic lipid selected from dLin-DMA (1,2-dilinoleyloxy-3-dimethylaminopropane), dLin-MC3-DM A (dilinoleylmethyl-4-dimethylaminobutyrate), dLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9 octadecenoyloxy)phenylacetoxy]ethyl}piperidinyl)ethyl]disulfide), and derivatives thereof. dLin-MC3-DMA and derivatives thereof are described, for example, in WO 2010/144740. DODMA and derivatives thereof are described, for example, in U.S. Pat. No. 7,745,651 and Mok et al. (1999), Biochimica et Biophysica Acta, 1419(2): 137-150. dLin-DMA and derivatives thereof are described, for example, in U.S. Pat. No. 7,799,565. dLin-KC2-DMA and derivatives thereof are described, for example, in U.S. Pat. No. 9,139,554. SS-OP (NOF America Corporation, White Plains, NY) is described, for example, at https://www.nofamerica.com/store/index.php?dispatch=products.view&product_id=962. Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis{2-[N-methyl-N-(a-D-tocopherolhemisuccinatepropyl)amino]ethyl}disulfide (SS-33/3AP05), Bis{2-[4-(a-D-tocopherolhemisuccinateethyl)piperidyl]ethyl}disulfide (SS33/4PE15), Bis{2-[4-(cis-9-octadecenoateethyl)-1-piperidinyl]ethyl}disulfide (SS18/4PE16), and Bis{2-[4-(cis,cis-9,12-octadecadienoateethyl)-1-piperidinyl]ethyl}disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to any of the polynucleotides of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-glycero-3-phosphate(Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol . . . )(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine(Sodium Salt) (DLPS-NA), 1,2-Dimyristoyl-sn-glycero-3-phosphate(SodiumSalt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(l-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine(Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LY S OPCM YRIS TIC), 1-Palmitoyl-sn-glycero-3-phosphocholine (LYSOPCPALMITIC), 1-Stearoyl-sn-glycero-3-phosphocholine (LYSOPC STEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)](Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG 1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells, and the characteristics of the nucleic acids and/or proteins to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and methods of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise a cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to the targeted cell, such as any cell described herein, e.g. a T cell.

In some embodiments, the vector exhibits immune cell or T cell tropism.

In some aspects, provided herein are pluralities of vectors that comprise any of the vectors described herein, and one or more additional vectors comprising one or more additional polynucleotides encoding an additional portion or an additional component of any of the DNA-targeting systems described herein, any of the gRNAs described herein, any of the fusion proteins described herein, or a portion or a component of any of the foregoing.

Provided are pluralities of vectors, that include: a first vector comprising any of the polynucleotides described herein; and a second vector comprising any of the polynucleotides described herein.

In some aspects, vectors provided herein may be referred to as delivery vehicles. In some aspects, any of the DNA-targeting systems, components thereof, or polynucleotides disclosed herein can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. Direct delivery of the RNP complex, including the DNA-binding domain complexed with the sgRNA, can eliminate the need for intracellular transcription and translation and can offer a robust platform for host cells with low transcriptional and translational activity. The RNP complexes can be introduced into the host cell by any of the methods known in the art.

In some embodiments, the method of introducing a nucleic acid into a host cell is a method comprising transient delivery, such as described in Section I.B.

Nucleic acids or RNPs of the disclosure can be incorporated into a host using virus-like particles (VLP). VLPs contain normal viral vector components, such as envelope and capsids, but lack the viral genome. For instance, nucleic acids expressing the Cas and sgRNA can be fused to the viral vector components such as gag and introduced into producer cells. The resulting virus-like particles containing the sgRNA-expressing vectors can infect the host cell for efficient editing.

Introduction of the complexes, polypeptides, and nucleic acids of the disclosure can occur by protein transduction domains (PTDs). PTDs, including the human immunodeficiency virus-1 TAT, herpes simplex virus-1 VP22, Drsophila Antennapedia Antp, and the poluarginines, are peptide sequences that can cross the cell membrane, enter a host cell, and deliver the complexes, polypeptides, and nucleic acids into the cell.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like, for example as described in WO 2017/193107, WO 2016/123578, WO 2014/152432, WO 2014/093661, WO 2014/093655, or WO 2021/226555.

Various methods for the introduction of polynucleotides are well known and may be used with the provided methods and compositions. Exemplary methods include those for transfer of polynucleotides encoding the DNA targeting systems provided herein, including via viral, e.g., retroviral or lentiviral, transduction, transposons, and electroporation.

In some embodiments, polynucleotides can be cloned into a suitable vector, such as an expression vector or vectors. The expression vector can be any suitable recombinant expression vector, and can be used to transform or transfect any suitable cell. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some embodiments, animal expression vectors include pEUK-Cl, pMAM and pMAMneo (Clontech). In some embodiments, a viral vector is used, such as a lentiviral or retroviral vector. In some embodiments, the recombinant expression vectors can be prepared using standard recombinant DNA techniques. In some embodiments, vectors can contain regulatory sequences, such as transcription and translation initiation and termination codons, which are specific to the type of host into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, or adeno-associated virus (AAV). In some embodiments, recombinant nucleic acids are transferred into cells (e.g. T cells) using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

In some embodiments, the vector is a lentiviral vector. In some embodiments, the lentiviral vector is an integrase-deficient lentiviral vector. In some embodiments, the lentiviral vector is a recombinant lentiviral vector. In some embodiments, the lentivirus is selected or engineered for a desired tropism (e.g. for T cell or immune cell tropism). Methods of lentiviral production, transduction, and engineering are known, for example as described in Kasaraneni, N. et al. Sci. Rep. 8(1):10990 (2018), Ghaleh, H. E. G. et al. Biomed. Pharmacother. 128:110276 (2020), and Milone, M. C. et al. Leukemia. 32(7):1529-1541 (2018). Additional methods for lentiviral transduction are described, for example in Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some embodiments, recombinant nucleic acids are transferred into cells (e.g. T cells) via electroporation {see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

### III. PHARMACEUTICAL COMPOSITIONS AND FORMULATIONS OF DNA-TARGETING SYSTEMS OR ENCODING POLYNUCLEOTIDES OR VECTORS

In some aspects, provided herein are compositions, such as pharmaceutical compositions and formulations for administration, that include any of the DNA-targeting systems described herein, for example in Section I, or any of the polynucleotides or vectors encoding the same, for example as described in Section II. In some aspects, the pharmaceutical composition contains one or more DNA-targeting systems provided herein or a component thereof. In some aspects, the pharmaceutical composition comprises one or more vectors, e.g., viral vectors that contain polynucleotides that encode one or more components of the DNA-targeting systems provided herein. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject or a cell to which the formulation would be administered.

In some embodiments, the pharmaceutical composition may further comprise a pharmaceutically acceptable excipient. The pharmaceutically acceptable excipient may be functional molecules as vehicles, adjuvants, carriers, or diluents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

In some embodiments, the transfection facilitating agent is a polyanion, polycation, including poly-L-glutamate (LGS), or lipid. In some embodiments, the transfection facilitating agent is poly-L-glutamate. In some embodiments, the transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the DNA-targeting system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent in a solvent, such as in admixture with a suitable carrier, diluent, or excipient such as sterile water, physiological saline, glucose, dextrose, or the like. The formulations to be used for in vivo or ex vivo administration or use are generally sterile. Sterility may be readily accomplished, e.g., by filtration through sterile filtration membranes.

The pharmaceutical composition in some embodiments contains components in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the composition can be administered to a subject by any suitable means, for example, by bolus infusion or by injection, e.g., by intravenous or subcutaneous injection. In some embodiments, a given dose is administered by a single bolus administration of the composition. In some embodiments, the composition is administered by multiple bolus administrations of the composition, for example, over a period of no more than 3 days, or by continuous infusion administration of the composition. In some embodiments, the composition is administered parenterally, for example by intravenous, intramuscular, subcutaneous, or intraperitoneal administration. In some embodiments, the composition is administered to a subject using peripheral systemic delivery by intravenous, intraperitoneal, or subcutaneous injection.

In some embodiments, the composition is contacted with our introduced into cells (e.g. primary T cells) from a subject ex vivo, and the cells are subsequently administered to the same subject or to a different subject.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

### IV. METHODS OF EPIGENETICALLY MODIFYING LYMPHOID CELLS, SUCH AS T CELLS, AND MODIFIED CELLS AND COMPOSITIONS THEREOF

Provided herein are modified lymphoid cells (e.g. T cells) that have one or more phenotypic and/or epigenetic modifications (also referred to as changes or alterations) in their epigenome. In some embodiments, the epigenetic change is a change relative to a comparable unmodified lymphoid cell. Reference to a comparable unmodified cell is understood to refer to the same or similar cell but that has not been introduced with a provided epigenome-modifying DNA-targeting system (such as any described in Section I) or that or that does not contain the same epigenetic changes (e.g. methylation or histone modification) of the target gene or regulatory region thereof.

In some embodiments, the epigenetic modification is decreased transcription of one or more genes (such as any described in Section I.B.2), or a phenotype or epigenetic modification resulting from the decreased transcription. In some embodiments, the phenotypic and/or epigenetic modification is increased transcription of one or more genes (such as any described in Section I.B.3), or a phenotype or epigenetic modification resulting from the increased transcription.

In some embodiments, the lymphoid cells that are modified by the provided DNA-binding systems can include T cells, NK cells, or NKT cells. Such cells can include cells that have been enriched or isolated from a primary population of cells from a subject, or can include any cells that have been differentiated from stem cells into such lymphoid cells and/or have been differentiated from progenitor cells, such as common lymphoid progenitors (CLPs). In some embodiments, the lymphoid cells are differentiated from stem cells, such as hematopoietic stem or progenitor cells, or progenitor cells. In some embodiments, the lymphoid cells are trans-differentiated from a non-pluripotent cell of non-hematopoietic lineage. In particular embodiments, the cells are modified T cells that have been modified by the provided DNA-binding systems.

Provided herein are modified T cells (e.g. CD4+ T cell or CD8+ T cell) that have one or more modifications (also referred to as changes or alterations) in their phenotype and/or epigenome. In some embodiments, the modification increases or promotes a phenotype in the T cell, such as any phenotype described herein, for example in Section I. In some embodiments, the modified cell is a modified T cell that has increased T cell effector function upon T cell stimulation. In some embodiments, the epigenetic change is a change relative to a comparable unmodified T cell. Reference to a comparable unmodified T cells is understood to refer to the same or similar T cell but that has not been introduced with a provided epigenome-modifying DNA-targeting system or that or that does not contain the same epigenetic changes (e.g. methylation or histone modification) of the target gene or regulatory region thereof.

In some embodiments, the epigenetic change comprises a change in at least one of: DNA accessibility, histone methylation, acetylation, phosphorylation, ubiquitylation, sumoylation, ribosylation, citrullination, and DNA methylation. In some embodiments, the epigenetic change is an altered DNA methylation of a target site in a target gene or a regulatory element thereof as described herein. In some embodiments, the epigenetic change is a histone modification of a target site in a target gene or a regulatory element thereof as described herein.

Provided herein are methods of phenotypically and/or epigenetically modifying a lymphoid cell or a population of lymphoid cells. The methods provided herein include use of one or more DNA-targeting system provided herein (e.g. as described in Section I), or polynucleotide or vector for delivery of same (e.g. as described in Section II) to the lymphoid cell or compositions of any of the foregoing. In some embodiments, the DNA-targeting system (or polynucleotides or vectors for delivery of same to the lymphoid cell or compositions of any of the foregoing) is contacted with a lymphoid cell or a population of lymphoid cells. In some embodiments, the contacting introduces the epigenome-modifying DNA-targeting system (or polynucleotides or vectors for delivery of same to the lymphoid cell or compositions of any of the foregoing) into the lymphoid cell, such as where it is able to translocate or localize to the nucleus of the lymphoid cell. In some embodiments, the methods increase the expression of one or more of the described target genes in lymphoid cells (e.g. T cells) in the population of cells. Also provided herein is a population of lymphoid cells containing a plurality of any of the provided modified lymphoid cells.

Provided herein are methods of phenotypically and/or epigenetically modifying a T cell or a population of T cells. In some embodiments, such methods promote a phenotype, such as any described herein, for example by altering the differentiation fate of the T cell. In some embodiments, such methods increase or enrich the phenotype among a population of T cells. Also provided herein are methods of promoting a phenotype in a T cell or a population of T cells. The methods provided herein include use of one or more DNA-targeting system provided herein, or polynucleotide or vector for delivery of same to the T cell or compositions of any of the foregoing. In some embodiments, the DNA-targeting system (or polynucleotides or vectors for delivery of same to the T cell or compositions of any of the foregoing) is contacted with a T cell or a population of T cells. In some embodiments, the contacting introduces the DNA-targeting system (or polynucleotides or vectors for delivery of same to the T cell or compositions of any of the foregoing) into the T cell, such as where it is able to translocate or localize to the nucleus of the T cell. In some embodiments, the methods promote a phenotype in the T cell or one or more T cells in the population. In some embodiments, the methods increase the percentage of T cells with the phenotype in the population of T cells.

In some embodiments, the epigenome-modifying DNA-targeting system (or polynucleotides or vectors for delivery of same to the T cell or compositions of any of the foregoing) can be introduced into a T cell or a population of T cells. In some embodiments, the epigenome-modifying DNA-targeting system (or polynucleotides or vectors for delivery of same to the T cell or compositions of any of the foregoing) can be cultured with a T cell or a population of T cells under conditions in which the epigenome-modifying DNA-targeting system (or polynucleotides or vectors for delivery of same to the T cell or compositions of any of the foregoing) will be introduced into or delivered to the T cell or one or more T cells in the population.

In some embodiments, the methods can be carried out in vitro. In other embodiments, the methods can be carried out ex vivo on T cells or a population containing T cells isolated from a subject. In other embodiments the epigenome-modifying DNA-targeting system (or polynucleotides or vectors for delivery of same to the T cell or compositions of any of the foregoing) can be administered to a subject, and then T cells can be isolated from the subject, such as for subsequent engineering. In still other embodiments the epigenome-modifying DNA-targeting system (or polynucleotides or vectors for delivery of same to the T cell or compositions of any of the foregoing) can be administered to a subject, and the T cells modified in vivo in the subject.

In any of the provided methods, the T cells can be T cells for use as a T cell based immunotherapy, such as for ACT. In certain embodiments, the population of lymphocytes is derived from peripheral blood mononuclear cells (PBMCs) isolated from the circulation of a subject. In certain embodiments, the population of lymphocytes is derived from lymphocytes isolated from a tumor (tumor infiltrating lymphocytes) of an individual. In certain embodiments, the population of lymphocytes comprises T lymphocytes (T cells). These cell populations can be heterogeneous comprised of a variety of lymphocytes, or they can be further subject to isolation/purification using density centrifugation (e.g., Percoll), fluorescently activated cell sorting (FACS), leukapheresis, or antibody based selection methods (positive or negative). T cells can be generally marked by expression of CD3, and further subdivided into cytotoxic (CD8+) or helper (CD4+) populations. When isolated/purified the cell population can comprise CD3+ cells at least 80%, 90%, or 95% pure. In certain embodiments, the population comprises CD3+, CD4+ T cells at least 80%, 90%, or 95% pure. In certain embodiments, the population comprises CD3+, CD8+ T cells at least 80%, 90%, or 95% pure.

In some embodiments, an isolated or purified cell population containing T cells can be further stimulated and, in some cases, expanded using standard methods, such as, incubation with anti-CD3 or CD28 antibody and/or co-culture with cytokines such as IL-2, IL-7 and/or IL-15. For instance, a population of isolated cells containing T cells can be further expanded using standard methods such as incubation with anti-CD3 or CD28 antibody and/or co-culture with cytokines such as IL-2, IL-7 and/or IL-15.

After the cells have been expanded the cells can comprise greater than 60%, 70%, 80%, 90%, or 95% CD3+ cells, CD3+CD4+ cells, or CD3+CD8+ cells. In certain embodiments, an aliquot of the cells can be tested for efficacy after expansion.

There are numerous methods available for isolating or expanding T cells or T-cell populations taken from an individual. Certain non-limiting methods of expanding and/or isolating T-cell populations are disclosed in U.S. Pat. Nos. 5,827,642; 6,316,257; 6,399,054; 7,745,140; 8,383,099; US 2003/0134341; US 2004/0241162; all of which are incorporated by reference herein in their entireties.

In some embodiments, the cells, such as T cells, may be further engineered with a recombinant antigen receptor, such as a chimeric antigen receptor (CAR) or an engineered TCR. In some embodiments, the cells may be stimulated (e.g. with anti-CD3 or CD28 antibody and/or IL-2, IL-7 and/or IL-15 cytokines) prior to engineering the cells, such as T cells, with the recombinant receptor. In some embodiments, the cells may be further expanded after engineering the cells, such as T cells, with the recombinant receptor.

In some embodiments, the cells, such as T cells, are engineered with a CAR. In some embodiments, the CAR is a chimeric receptor that contains an extracellular antigen targeting domain (e.g., an antibody Fab or single chain variable fragment) fused to a transmembrane domain, and an intracellular signaling domain that induces activation of the cell, such as T cell, upon interaction of the CD3 zeta signaling domain and a costimulatory signaling domain. Non-limiting examples of a costimulatory signaling domain include a CD28 intracellular domain or a 4-1BB intracellular domain. In some embodiments, the extracellular targeting domain is specific for a tumor associated antigen (TAA). Non-limiting examples of TAAs include, for example, CD19, glioma-associated antigen, carcinoembryonic antigen (CEA), f-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, or CD37. Current FDA approved CAR T cell therapies include CAR-T cell therapies for targeting CD19, such as for treating lymphoma, including axicabtagene ciloleucel (Yescarta™), tisagenlecleucel (Kymriah™) and lisocabtagene maraleucel (Breyanzi®). Current FDA approved CAR T cell therapies also include CAR-T cell therapies for targeting BCMA, such as for treating multiple myeloma, including idecabtagene vicleucel (Abecma®) and ciltacabtagene autoleucel (Carvkti™). CAR constructs and methods of their use are described in, by way of non-limiting example, US20130287748A1; US 2014/0234348A1; or US 2014/0050708, all of which are incorporated by reference herein in their entirety.

In some embodiments, the T cells are engineered with a TCR. In some embodiments, the TCR is specific for a TAA. In particular embodiments, the TCR is a recombinant TCR that is introduced into the T cell and is heterologous to the T cell. The TCR can be specific for a TAA, such as, glioma-associated antigen, carcinoembryonic antigen (CEA), f-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, or CD37. In some embodiments, the TAA is cancer-testis (CT) antigen. In some embodiments, the TAA is neoantigen or oncoviral antigen. Exemplary target antigens of a TCR include, but are not limited to, a MAGE (e.g. MAGE-A1, MAGE-A2, MAGE-A3, MAGE-A4, MAGE-A6, MAGE-A10, and MAGE-A12, such as MAGE-A4, MAGE-A10, MAGE-A3/MAGE-A6), glycoprotein (gp100), melanoma antigen recognized by T cells (MART-1), Wilms tumor 1 (WT1), PRAME, NY-ESO-1, mesothelin, α-fetoprotein (AFP) or Human papillomavirus (HPV) E6 protein and HPV E7 protein. TCRs and method of their use in ACT are known and described in, by way of non-limiting example, Tsimberidou, A M., et al. J Hematol Oncol 14, 102 (2021).

In some embodiments, the recombinant antigen receptor, such as a CAR or TCR, can be engineered into the cell, such as T cell, by viral transduction of a nucleic acid encoding ther recombinant antigen receptor into a primary T-cell population, using for example a retroviral, adenoviral, or AAV-vector; or transfection via a lipid-based reagent or electroporation. In some embodiments, the methods described herein involve engineering a population of lymphoid cells, such as a T-cell population, with the recombinant antigen receptor (e.g. CAR or TCR) before contacting the population with the epigenome-modifying DNA-targeting system (or polynucleotides or vectors for delivery of same to the T cell or compositions of any of the foregoing). In certain embodiments, the methods involve engineering a population of lymphoid cells, such as a T-cell population, with the recombinant antigen receptor (e.g. CAR or TCR) after contacting the cells with the epigenome-modifying DNA-targeting system (or polynucleotides or vectors for delivery of same to the T cell or compositions of any of the foregoing). In some embodiments, when the engineered lymphocytes, such as T cells (e.g. CAR-T cells or eTCR-T cells), are generated from a primary lymphocyte population the cells are often autologous to the patient being treated. In some embodiments, a process for engineering T cells with a recombinant receptor (e.g. CAR or TCR) includes isolating the T cells from a subject, stimulating the T cells in culture using a conventional method such as CD3/CD28 antibodies prior to transduction with a viral vector encoding the recombinant antigen receptor (e.g. CAR or TCR) and, if necessary, expanding the cells to generate sufficient cells for subsequent administration to the subject. In some embodiments, contacting the T cells with the epigenome-modifying DNA-targeting system (or polynucleotides or vectors for delivery of same to the T cell or compositions of any of the foregoing) can be prior to or during any step of stimulating, transducing or expanding the T cells.

In certain embodiments, an isolated or purified cell population containing T cells is incubated with peptide antigen and, in some cases also irradiated feeder cells or other agents, to expand one or more T cells of a certain antigen specificity. In certain embodiments, the peptide antigen comprises a tumor associated antigen. In some embodiments, such an isolated or purified cell population includes tumor infiltrating lymphocytes (TILs) such as for TIL therapy. In some embodiments, the population can be stimulated or activated by a specific tumor-associated antigen either before or after contact with epigenome-modifying DNA-targeting system (or polynucleotides or vectors for delivery of same to the T cell or compositions of any of the foregoing). A tumor associated antigen (TAA) is one that is exclusively expressed or highly expressed by a neoplastic cell compared to a normal cell of the same origin. Known tumor-associated antigens include, for example, glioma-associated antigen, carcinoembryonic antigen (CEA), f-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, CD37, or patient specific idiotype. In certain embodiments, greater than 50%, 60%, 70%, 80%, 90%, or 95% of the T-cell population can be specific for a tumor associated antigen (as defined by tetramer staining for example). In certain embodiments, the T-cell population may not be stimulated with TAA, but may possess specificity for the TAA, as indicated for example, by tetramer staining.

In some embodiments, the population of cells, such as T cells, may be autologous to a subject to be treated. For instance, the population of lymphoid cells, such as T-cell populations, to be contacted with an epigenome-modifying DNA-targeting system (or polynucleotides or vectors for delivery of same to the T cell or compositions of any of the foregoing) can be derived from an individual that will ultimately be treated with the cell-based immunotherapeutic (e.g., an autologous population). In certain embodiments, when an autologous cell population is used the cell population has been contacted in vitro with the epigenome-modifying DNA-targeting system (or polynucleotides or vectors for delivery of same to the cells, such as T cell, or compositions of any of the foregoing). In certain embodiments, when an autologous cell population is used a subject to be treated has been administered the epigenome-modifying DNA-targeting system (or polynucleotides or vectors for delivery of same to the cell, such as T cell, or compositions of any of the foregoing) on one or more occasions prior to isolation of the cell population.

In other embodiments, the population of lymphoid cells, such as population of T cells, may be for allogeneic therapy. In such an example, the population of lymphoid cells, such as T-cell population, to be contacted with an epigenome-modifying DNA-targeting system (or polynucleotides or vectors for delivery of same to the cells, such as T cell, or compositions of any of the foregoing) can be derived from a different individual (e.g., a heterologous population) than is to be treated. In certain embodiments, when a heterologous cell population is used it is from an HLA matched individual (e.g., syngeneic) or an HLA mismatched individual (e.g., allogeneic). In certain embodiments, when a heterologous cell population is used it is from an HLA mismatched donor. In certain embodiments, when a heterologous cell population is used it is a T cell line that can be established from an autologous or heterologous source.

T cell populations can also be derived from hematopoietic stem cells (HSCs) or induced pluripotent stem cells (iPSCs) using methods known in the art. In certain embodiments, T-cell populations are derived/differentiated from iPSCs. The source of the iPSCs can be either autologous or heterologous. In certain embodiments, T-cell populations are derived/differentiated from (HSCs) cells. The source of the HSCs can be either autologous or heterologous.

In some embodiments, the modified T cell comprises an epigenetic or phenotypic modification resulting from being contacted by any of the DNA-targeting systems described herein (for example in Section I), including any including any gRNA described herein (for example in Section I.B.ii).

In some embodiments, the modified T cell is derived from a cell from a subject, such as a primary T cell, a T cell progenitor, a pluripotent stem cell, or an induced pluripotent stem cell. In some embodiments, the modified T cell is derived from a primary T cell.

In some embodiments, the modified T cell is derived from a subject. In some embodiments, the subject has or is suspected of having cancer.

In some aspects, provided herein are methods for modulating (e.g. increasing or decreasing transcription of) the expression of a gene in a cell (e.g. a T cell), such as a target gene as described in Section I.B. In some embodiments, the method includes: introducing into the cell any of the DNA-targeting systems described herein, or a polynucleotide or vector containing or encoding the same.

In some embodiments, the expression of the one or more genes, such as one or more target genes described in Section I.B.2, is decreased in comparison to a comparable unmodified cell (e.g. T cell) not subjected to the method, i.e. not contacted or introduced with the DNA-targeting system described herein. In some embodiments, the expression of the one or more genes is decreased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, 5-fold, 10-fold, 20-fold, 30-fold, 40-fold, 50-fold, 100-fod, 200-fold, 300-fold, 400-fold, 500-fold, 1000-fold or greater. In some embodiments, the expression is stably decreased or transiently decreased. In some embodiments, the decreased expression of the one or more genes promotes increased T cell effector function in a T cell.

In some embodiments, the expression of the one or more genes, such as one or more target genes described in Section I.B.3, is increased in comparison to a comparable unmodified cell (e.g. T cell) not subjected to the method, i.e. not contacted or introduced with the DNA-targeting system described herein. In some embodiments, the expression of the one or more genes is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, 5-fold, 10-fold, 20-fold, 30-fold, 40-fold, 50-fold, 100-fod, 200-fold, 300-fold, 400-fold, 500-fold, 1000-fold or greater. In some embodiments, the expression is stably increased or transiently increased. In some embodiments, the increased expression of the one or more genes promotes increased T cell effector function in a T cell.

In some embodiments, the one or more modifications in the epigenome of the modified lymphoid cells, such as a T cell, NK cell or NK T cell, or any cells that have been differentiated from stem cells into such lymphoid cells and/or have been differentiated from progenitor cells, such as common lymphoid progenitors (CLPs), is by targeting one or more genes as described herein with a provided epigenome-modifying DNA-targeting system to change the epigenome of the cell. In some embodiments, the one or more modifications in the epigenome of the modified T cell is by targeting one or more genes as described herein with a provided epigenome-modifying DNA-targeting system to change the epigenome of the T cell. In some embodiments, the modified cell, such as modified T cell, includes an epigenetic change in a gene selected from the list consisting of: CBLB, CCNC, CD5, CISH, DGKZ, ELOB, FAS, Fli1, GATA3, KDM1A, MED12, MYB, PRDM1, TGFBR2, and RASA2. In some embodiments, the modified cell, such as modified T cell, includes an epigenetic change in a gene selected from the list consisting of: BATF, CD28, EOMES, IL-2, IL2RB, IRF4, LAT, LCP2, TBX21, and VAV1

In some embodiments, the modified cell, such as modified T cell has decreased expression of one or more genes selected from the list consisting of: CBLB, CCNC, CD5, CISH, DGKZ, ELOB, FAS, Fli1, GATA3, KDM1A, MED12, MYB, PRDM1, TGFBR2, and RASA2. In some embodiments, the expression of the gene in the modified cell is decreased 1.5-fold or more compared to the expression of the same gene in a comparable unmodified T cell, such as decreased by at or about or greater than 2-fold, 3-fold, 4-fold, 5-fold, 6-fold, 7-fold, 8-fold, 9-fold, 10-fold or more.

In some embodiments, the modified T cell exhibits decreased expression of one or more genes to promote increased T cell effector function upon T cell stimulation, in comparison to a comparable unmodified T cell, such as a T cell not subjected to the method, i.e. not contacted or introduced with the DNA-targeting system described herein. In some embodiments, the modified T cell has decreased expression of one more genes selected from the list consisting of: CBLB, CCNC, CD5, CISH, DGKZ, ELOB, FAS, Fli1, GATA3, KDM1A, MED12, MYB, PRDM1, TGFBR2, and RASA2. In some embodiments, the expression of the gene in the modified T cell is decreased 1.5-fold or more compared to the expression of the same gene in a comparable unmodified T cell, such as decreased by at or about or greater than 2-fold, 3-fold, 4-fold, 5-fold, 6-fold, 7-fold, 8-fold, 9-fold, 10-fold or more.

In some embodiments, the modified cell, such as modified T cell has increased expression of one or more genes selected from the list consisting of: BATF, CD28, EOMES, IL-2, IL2RB, IRF4, LAT, LCP2, TBX21, and VAV1. In some embodiments, the expression of the gene in the modified cell is increased 1.5-fold or more compared to the expression of the same gene in a comparable unmodified T cell, such as increased by at or about or greater than 2-fold, 3-fold, 4-fold, 5-fold, 6-fold, 7-fold, 8-fold, 9-fold, 10-fold or more.

In some embodiments, the modified T cell exhibits increased expression of one or more genes to promote increased T cell effector function upon T cell stimulation, in comparison to a comparable unmodified T cell, such as a T cell not subjected to the method, i.e. not contacted or introduced with the DNA-targeting system described herein. In some embodiments, the modified T cell has increased expression of one more genes selected from the list consisting of: BATF, CD28, EOMES, IL-2, IL2RB, IRF4, LAT, LCP2, TBX21, and VAV1. In some embodiments, the expression of the gene in the modified T cell is increased 1.5-fold or more compared to the expression of the same gene in a comparable unmodified T cell, such as increased by at or about or greater than 2-fold, 3-fold, 4-fold, 5-fold, 6-fold, 7-fold, 8-fold, 9-fold, 10-fold or more.

Also provided herein is a population of T cells containing a plurality of any of the provided modified T cells. In some embodiments, the population of T cells is enriched for cells that have a phenotype. In some embodiments, the phenotype is increased T cell effector function upon T cell stimulation. In some embodiments, the population of T cells contains greater than at or about 40%, greater than at or about 50%, greater than at or about 60%, greater than at or about 70%, greater than at or about 80% or greater than at or about 90% of cells that have the phenotype. In some embodiments, the population of T cells has an increased percentage of cells of the phenotype compared to a comparable population of unmodified cell (e.g. T cell) not subjected to the method, i.e. not contacted or introduced with the DNA-targeting system described herein. In some embodiments, the population of T cells has an increased percentage of cells having the phenotype compared to the percentage of cells having the phenotype prior to contacting the population of T cells with the DNA-targeting system described herein. In some embodiments, the increased percentage is by at or about or greater than 1.5-fold, 2-food, 3-fold, 4-fold, 5-fold or more. In some aspects, the phenotype comprises expression of IL-2, IFN-gamma, and/or TNF-alpha.

In some embodiments, the population of cells, such as population of T cells, contains at least at or about 40%, at least at or about 50%, at least at or about 60%, at least at or about 70%, at least at or about 80% or at least at or about 90% of cells that have an epigenetic change (e.g. methylation or histone modification) at or near a target site in a target gene. In some embodiments, the population of cells, such as population of T cells, has an increased percentage of cells (e.g. T cells) that have an epigenetic change at or near a target site in a target gene compared to a comparable population of unmodified cell (e.g. T cell) not subjected to the method, i.e. not contacted or introduced with the DNA-targeting system described herein. In some embodiments, the epigenetic change is a change, such as on average in cells in the population, of at least one of: DNA accessibility, histone methylation, acetylation, phosphorylation, ubiquitylation, sumoylation, ribosylation, citrullination, and DNA methylation, compared to a comparable population of unmodified cell (e.g. T cell) not subjected to the method, i.e. not contacted or introduced with the DNA-targeting system described herein. In some embodiments the population of cells is a population of T cells. In some embodiments, the population of T cells contains at least at or about 40%, at least at or about 50%, at least at or about 60%, at least at or about 70%, at least at or about 80% or at least at or about 90% of cells that have an epigenetic change (e.g. methylation or histone modification) at or near a target site in a target gene and exhibits increased T cell effector function upon stimulation.

In some embodiments, provided herein is a population of cells that contains at least at or about 40%, at least at or about 50%, at least at or about 60%, at least at or about 70%, at least at or about 80% or at least at or about 90% of cells that have an epigenetic change (e.g. methylation or histone modification) at a target gene and that are IL-2+, or that are IL-2- and IFN-gamma+. In some embodiments, the population of cells is a population of T cells.

In some embodiments, the modified T cell or a composition containing a plurality of modified T cells is capable of a stronger and/or more persistent immune response (e.g. an anti-tumor immune response in vivo), in comparison to a comparable unmodified T cell or composition of unmodified T cells. In some embodiments, a subject having received administration of a composition of T cells containing provided modified T cells as a T cell therapy, e.g. CAR-T cell, is monitored for the presence, absence or level of T cells of the therapy in the subject, such as in a biological sample of the subject, e.g. in the blood of the subject. In some embodiments, the provided methods result in T cells of the adoptive T cell therapy with increased T cell effector function, including increased cytokine production, proliferation, killing of target cells, and/or persistence. In some embodiments, the T cell effector function of the adoptively transferred T cells, such as CAR-expressing T cells, in the subject is greater as compared to that which would be achieved by alternative methods, such as those involving administration of a T cell therapy but without having been treated or contacted with a provided DNA-targeting system. In some embodiments, the T cell effector function, such as any described herein, is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR or recombinant TCR) or other surrogate marker expressed by T cells of the therapy in the blood or serum or organ or tissue (e.g., disease site) of the subject. In some aspects, persistence is quantified as copies of DNA or plasmid encoding the recombinant receptor (e.g., CAR or recombinant TCR) or surrogate marker per microgram of DNA or per microliter of the sample, e.g., of blood or serum, or per total number of peripheral blood mononuclear cells (PBMCs) or white blood cells or T cells per microliter of the sample. In some embodiments, flow cytometric assays using antibodies specific for the recombinant receptor or surrogate marker also can be performed to detect the adoptively transferred cells. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of any marker (e.g. surrogate marker, CAR, recombinant TCR) known to be expressed by the adoptively transferred T cells but not endogenous T cells can be used to distinguish the administered cells from endogenous cells in a subject.

In some embodiments, the modified T cell or a composition containing a plurality of modified T cells, such a produced by any of the provided methods, exhibits a reduction in features associated with T cell exhaustion in comparison to a comparable unmodified T cell or composition of unmodified T cells. In some embodiments, the T cells, such as a composition containing a modified T cell or a composition of modified T cell provided herein, exhibits reduced exhaustion following long-term stimulation with antigen, either in vitro or in vivo. For example, an assay for assessing long-term stimulation with antigen may include a serial restimulation assay (see e.g. Jensen et al. Immunol. Rev. 2014; 257:127-144; Win et al. Journal of Immunotherapy, 2020; 43:107-120). In some embodiments, the percentage of T cells that exhibit an exhausted phenotype is reduced 2-fold, 3-fold, 4-fold, 5-fold, 6-fold, 7-fold, 8-fold, 9-fold, 10-fold or more.

Various assays are known and can be used to assess or determine if the T cells exhibit features of exhaustion or a reduction in features of exhaustion in comparison to a comparable unmodified T cell or composition of unmodified T cells. In some cases, exhaustion can be assessed by monitoring loss of T cell function, such as reduced or decreased antigen-specific or antigen receptor-driven activity, such as a reduced or decreased ability to produce cytokines or to drive cytolytic activity against target antigen. In some cases, exhaustion also can be assessed by monitoring expression of surface markers on T cells (e.g. CD4 and/or CD4 T cells) that are associated with an exhaustion phenotype. In some embodiments, the exhaustion marker is any one or more of PD-1, CTLA-4, TIM-3, LAG-3, BTLA, 2B4, CD160, CD39, VISTA, and TIGIT Among exhaustion markers are inhibitory receptors such as PD-1, CTLA-4, LAG-3 and TIM-3. In certain embodiments, the biological activity of the cells is measured by assaying expression and/or secretion of one or more cytokines, such as CD107a, IFNy, IL-2, GM-CSF and TNFa, and/or by assessing cytolytic activity. In some embodiments, assays for the activity, phenotypes, proliferation and/or function of the T cells include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of ³H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimidyl ester (CFSE), CellTrace Violet, or membrane dye PKH26.

Also provided herein are compositions containing a modified lymphoid cell or a plurality of or population of modified lymphoid cells provided herein, such as modified T cells, NK cell, NKT cell, or such cells that are modified and have been differentiated from stem cells into such lymphoid cells and/or have been differentiated from progenitor cells, such as common lymphoid progenitors (CLPs). Also provided herein are compositions containing a modified T cell or a plurality of or population of modified T cells provided herein. In some embodiments, the composition is a pharmaceutical composition and further contains a pharmaceutically acceptable carrier. Such compositions can be used in accord with the provided methods, and/or with the provided articles of manufacture or compositions, such as in the prevention or treatment of diseases, conditions, and disorders, or in detection, diagnostic, and prognostic methods.

Among the compositions are pharmaceutical compositions and formulations for administration, such as for adoptive cell therapy. In some embodiments, the engineered cells are formulated with a pharmaceutically acceptable carrier.

A pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for T cells, such as a saline solution, a dextrose solution or a solution comprising human serum albumin.

In some embodiments, the pharmaceutically acceptable carrier or vehicle for such compositions is any non-toxic aqueous solution in which the cells, such as T cells, can be maintained, or remain viable, for a time sufficient to allow administration of live cells, such as live T cells. For example, the pharmaceutically acceptable carrier or vehicle can be a saline solution or buffered saline solution. The pharmaceutically acceptable carrier or vehicle can also include various bio materials that may increase the efficiency of the cells, such as T cells. Cell vehicles and carriers can, for example, include polysaccharides such as methylcellulose (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P(NIPAM-co-AA) (Y. H. Bae, B. Vernon, C. K. Han, S. W. Kim, J. Control. Release 53, 249, 1998; H. Gappa, M. Baudys, J. J. Koh, S. W. Kim, Y. H. Bae, Tissue Eng. 7, 35, 2001, each of which is incorporated herein by reference in its entirety), as well as Poly(oxyethylene)/poly(D,L-lactic acid-co-glycolic acid) (B. Jeong, K. M. Lee, A. Gutowska, Y. H. An, Biomacromolecules 3, 865, 2002, which is incorporated herein by reference in its entirety), P(PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lee, Po-Han Kung and Yu-Der Lee, Carbohydrate Polymers, 61, 348, 2005, which is incorporated herein by reference in its entirety), collagen (Lee C R, Grodzinsky A J, Spector M., Biomaterials 22, 3145, 2001, which is incorporated herein by reference in its entirety), alginate (Bouhadir K H, Lee K Y, Alsberg E, Damm K L, Anderson K W, Mooney D J. Biotech Prog 17, 945, 2001; Smidsrd O, Skjak-Braek G., Trends Biotech, 8, 71, 1990, each of which is incorporated herein by reference in its entirety).

In some embodiments, the cells, such as T cells, can be present in the composition in an effective amount. In some embodiments, the composition contains an effective amount of T cells, such as containing modified T cells produced by the provided methods. In some embodiments, the composition of T cells are enriched in T cells with increased T cell effector function. An effective amount of cells can vary depending on the patient, as well as the type, severity and extent of disease. Thus, a physician can determine what an effective amount is after considering the health of the subject, the extent and severity of disease, and other variables.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided lymphoid cells, such as modified cells including such lymphoid cells produced by any of the provided methods. In some embodiments, the lymphoid cells are cryopreserved in a serum-free cryopreservation medium.

Also provided herein are compositions that are suitable for cryopreserving the provided T cells, such as modified T cells including T cells produced by any of the provided methods. In some embodiments, the T cells are cryopreserved in a serum-free cryopreservation medium.

In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO).In some embodiments, compositions formulated for cryopreservation can be stored at low temperatures, such as ultra low temperatures, for example, storage with temperature ranges from −40° C. to −150° C., such as or about 80° C.±6.0° C.

In some embodiments, the cryopreserved cells, such as T cells, are prepared for administration by thawing. In some cases, the cells, such as T cells, can be administered to a subject immediately after thawing. In such an embodiment, the composition is ready-to-use without any further processing. In other cases, the cells, such as T cells, are further processed after thawing, such as by resuspension with a pharmaceutically acceptable carrier, incubation with an activating or stimulating agent, or are activated washed and resuspended in a pharmaceutically acceptable buffer prior to administration to a subject.

### V. METHODS OF TREATMENT

Provided herein are methods of treatment, e.g., including administering any of the compositions described herein, such as DNA-targeting systems (e.g. as described in Section I), polynucleotides and vectors (e.g. as described in Section II), and pharmaceutical compositions and formulations (e.g. as described in Section III). In some aspects, also provided are methods of administering any of the compositions described herein to a subject, such as a subject that has a disease or disorder. The compositions, such as pharmaceutical compositions, described herein are useful in a variety of therapeutic, diagnostic and prophylactic indications. For example, the compositions are useful in treating a variety of diseases and disorders in a subject. Such methods and uses include therapeutic methods and uses, for example, involving administration of the compositions, to a subject having a disease, condition, or disorder, such as a tumor or cancer. In some embodiments, the compositions are administered in an effective amount to effect treatment of the disease or disorder. Uses include uses of the compositions in such methods and treatments, and in the preparation of a medicament in order to carry out such therapeutic methods. In some embodiments, the methods are carried out by administering the compositions to the subject having or suspected of having the disease or condition. In some embodiments, the methods thereby treat the disease or condition or disorder in the subject. Also provided are therapeutic methods for administering the cells and compositions to subjects, e.g., patients.

In some embodiments, the compositions include a DNA-targeting system provided herein (e.g. as described in Section I), or a polynucleotide or vector encoding the same (e.g. as described in Section II), in which delivery of the composition to a subject modulates one or more activities or function of lymphoid cells in a subject to thereby treat a disease or condition. For instance, in some embodiments, the subject has been previously treated with an adoptive cell therapy involving administration of a population of lymphoid cells (e.g. T cell, NK or NKT cell therapy, including primary cells or cells differentiated from stem cells or progenitor cells such as common lymphoid cells) for treating a disease or disorder, and administration of a provided DNA-targeting system, or a polynucleotide or vector encoding the same, modulates a phenotype or function of the adoptively transferred cells in the subject for treating the disease or condition. In some embodiments, the cells may include a T cell infiltrating lymphocyte (TIL) therapy. In som embodiments, the cells are engineered with an antigen receptor, such as a chimeric antigen receptor or T cell receptor, targeting an antigen associated with the disease or condition. In some embodiments, administration or use of a composition that includes a DNA-targeting system provided herein, or a polynucleotide or vector encoding the same, increases expression of one or more target genes as described herein in the lymphoid cell.

In some embodiments, the compositions include a DNA-targeting system provided herein, or a polynucleotide or vector encoding the same, in which delivery of the composition to a subject modulates one or more activities or function of T cells in a subject to thereby treat a disease or condition. For instance, in some embodiments, the subject has been previously treated with an adoptive T cell therapy for treating a disease or disorder, such as a TIL therapy or a CAR- or TCR-engineered T cell therapy, and administration of a provided DNA-targeting system, or a polynucleotide or vector encoding the same, modulates a phenotype or function of the adoptively transferred T cells in the subject for treating the disease or condition. In some embodiments, administration or use of a composition that includes a DNA-targeting system provided herein, or a polynucleotide or vector encoding the same, increases expression of one or more genes that promotes a phenotype comprising increased T cell effector function upon stimulation in a T cell. In some embodiments, the percentage of T cells of the adoptive cell therapy in the subject that has the phenotype is increased in the subject compared to prior to the administration of the composition that includes the DNA-targeting system or a polynucleotide or vector encoding the same.

In some aspects, also provided herein are methods of promoting a phenotype in a T cell or in T cells in a subject, according to any description of a phenotype comprising increased T cell effector function provided herein. In some embodiments, the percentage of T cells that have the phenotype is increased in the subject compared to prior to the administration of the composition containing the DNA-targeting system or a polynucleotide or vector encoding the same. In some embodiments, the T cells include T cells of a previously administered adoptive cell therapy, such as CAR-expressing or recombinant TCR-expressing T cells.

In some embodiments, the methods of administering a composition containing the DNA-targeting system or a polynucleotide or vector encoding the same to a subject as provided herein are carried out in vivo (i.e. in a subject).

In some embodiments, methods of contacting a cell (e.g. T cell) with a composition containing the DNA-targeting system or a polynucleotide or vector encoding the same provided herein are carried out ex vivo on a cell from a subject, for example a primary T cell, a T cell progenitor, a pluripotent stem cell, or an induced pluripotent stem cell, such as by methods described in Section IV. In some embodiments, the methods provided herein are carried out ex vivo on a primary T cell. In some embodiments, when the methods are carried out ex vivo, such as by methods described in Section IV, the provided methods of treatment include administering a dose of the modified cells (e.g. T cells) to the subject for treating a disease or disorder. In some embodiments, the modified cells are modified T cells that have been epigenetically modified by the provided methods and enriched in T cells that have the phenotype comprising increased T cell effector function.

In some embodiments, also provided herein are methods include administering to a subject a composition containing an epigenetically modified cells (e.g. epigenetically modified T cells) provided herein. In some embodiments, administration of an effective dose of epigenetically modified cells treats a disease or condition in the subject. In some embodiments, the dose of epigenetically modified cells (e.g. T cells) is for use in adoptive cell therapy. In some embodiments, the epigenetically modified cell is a tumor infiltrating lymphocyte (TIL) therapy. In some embodiments, the epigenetically modified cell is a T cell that has been engineered with a recombinant antigen receptor, such as a chimeric antigen receptor or a T cell receptor (TCR) in which targeting of the antigen by the recombinant receptor (e.g. CAR or TCR)-engineered T cell treats the disease or condition.

In some aspects, provided is a method for treating a disease in a subject, comprising administering to the subject a cellular composition that comprises any of the modified T cells described herein. In some aspects, the modified cell (e.g. T cell) is one that has been obtained from or derived from a cell from a subject and modified by contacting the cells with a provided DNA-targeting system or a polynucleotide or vector encoding the same. In some aspects, the modified cell (e.g. T cell) is obtained from or derived from a cell from a subject, and administered to the same subject (i.e. autologous adoptive cell therapy). In some aspects, the modified cell (e.g. T cell) is obtained from or derived from a cell from a subject, and administered to a different subject (i.e. allogeneic adoptive cell therapy).

In some embodiments, the methods of treatment or uses involve administration to a subject of an effective amount of a composition containing modified cells (e.g. T cells) provided herein. In some embodiments, the effective amount may include a dose of cells (e.g. T cells) of the composition from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of such. In some embodiments, the provided compositions containing modified cells (e.g. T cells) provided herein can be administered to a subject by any convenient route including parenteral routes such as subcutaneous, intramuscular, intravenous, and/or epidural routes of administration. In particular embodiments, the modified T cells are administered by intravenous infusion to the subject.

In some embodiments, the methods of treatment or uses involve administration to a subject of an effective amount of a composition containing modified cells T cells provided herein, including any such composition that is enriched in T cells having a phenotype comprising increased T cell effector function as produced by the provided methods. In some embodiments, the effective amount may include a dose of T cells of the composition from at or about 10⁵ to at about 10¹², or from at or about 105 and at or about 10⁸, or from at or about 10⁶ and at or about 10¹², or from at or about 10⁸ and at or about 10¹¹, or from at or about 10′ and at or about 10¹⁰ of such. In some embodiments, the provided compositions containing modified T cells provided herein can be administered to a subject by any convenient route including parenteral routes such as subcutaneous, intramuscular, intravenous, and/or epidural routes of administration. In particular embodiments, the modified T cells are administered by intravenous infusion to the subject.

In some embodiments, the provided methods can be used to treat any disease or disorder in which treatment is contemplated by the adoptive cell therapy. For instance, in the case of a CAR or a TCR, the disease or condition to be treated is any disease or condition that is associated with expression of an antigen that is recognized or targeted by the CAR- or TCR-cell therapy. In other embodiments, for the case of a TIL therapy the disease or condition is a tumor, and typically is a tumor present in the subject from which the TIL therapy was derived. Methods for adoptive T cell therapy are known, see e.g. for CAR-T cell therapy: U.S. Pat. Nos. 7,446,190, 7,741,465, WO2016109410, WO2012079000, WO2017015427, WO2017040930, WO2017149515, WO201716568; WO2017181119; for TCR-T cell therapy: US20160137715, US 20190321478; WO2015184228, WO2017158103; for TIL therapy: US2003194804, US20120244133, US20210220457, US20210189339, U.S. Pat. Nos. 5,126,132, and 11,083,752. Any of such methods or other similar methods can be used in connection with the present disclosure. In some embodiments, the provided methods are performed ex vivo during the process of manufacturing or preparing the T cells for adoptive transfer to a subject, such as using methods described in Section IV, and then the modified T cells are administered to the subject for treating a disease or disorder. In other embodiments, the provided methods are performed by administering to the subject a composition containing the DNA-targeting system or a polynucleotide or vector encoding the same in combination with adoptive transfer of a T cell therapy. In such methods, the composition containing the DNA-targeting system or a polynucleotide or vector encoding the same is administered prior to, simultaneously with or after administration of the adoptive T cell therapy.

In some embodiments, the disease, condition, or disorder to be treated is cancer, viral infection, autoimmune disease, or graft-versus-host disease. In some embodiments, the subject to be treated has undergone or is expected to undergo organ transplantation.

In some embodiments, the disease or condition to be treated is a cancer. In some embodiments, the cancer is a hematologic cancer. In some embodiments, the cancer is a B cell malignancy. In some embodiments, the cancer is a myeloma, a lymphoma or a leukemia. In some embodiments, the methods can be used to treat a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM).

In some embodiments, the cancer is a solid tumor cancer. In some embodiments, the cancer is a bladder, lung, brain, melanoma (e.g. small-cell lung, melanoma), breast, cervical, ovarian, colorectal, pancreatic, endometrial, esophageal, kidney, liver, prostate, skin, thyroid, or uterine cancers. In some embodiments, the cancer is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In some aspects, the provided methods can further include administering one or more lymphodepleting therapies, such as prior to or simultaneous with initiation of administration of the adoptive T cell therapy, such as a composition containing modified T cells provided herein. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). In some embodiments, the lymphodepleting therapy includes combinations of cyclosporine and fludarabine.

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent, such as a lymphodepleting or chemotherapeutic agent, such as cyclophosphamide, fludarabine, or combinations thereof, at least 2 days prior, such as at least 3, 4, 5, 6, or 7 days prior, to the first or subsequent dose. In some embodiments, the subject is administered a preconditioning agent, such as a lymphodepleting or chemotherapeutic agent, such as cyclophosphamide, fludarabine, or combinations thereof, no more than 7 days prior, such as no more than 6, 5, 4, 3, or 2 days prior, to the administration of the dose of cells. In some embodiments, the subject is administered a preconditioning agent, such as a lymphodepleting or chemotherapeutic agent, such as cyclophosphamide, fludarabine, or combinations thereof, no more than 14 days prior, such as no more than 13, 12, 11, 10, 9 or 8 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In some embodiments, prior to the administration of adoptive T cell therapy, such as a composition containing modified T cells described herein, the subject has received a lymphodepleting therapy. In some embodiments, the lymphodepleting therapy includes fludarabine and/or cyclophosphamide. In some embodiments, the lymphodepleting includes the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

In some embodiments, the lymphodepleting therapy includes fludarabine and cyclophosphamide. In some embodiments, the lymphodepleting therapy includes the administration of fludarabine at or about 30 mg/m² body surface area of the subject, daily, and cyclophosphamide at or about 300 mg/m² body surface area of the subject, daily, each for 2-4 days, optionally 3 days.

In some embodiments, the administration of the preconditioning agent prior to infusion of the dose of cells improves an outcome of the treatment. For example, in some aspects, preconditioning, such as a lymphodepleting or chemotherapeutic agent, such as cyclophosphamide, fludarabine, or combinations thereof, improves the efficacy of treatment with the dose or increases the persistence of the T cells in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the T cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines or other effector molecules, such as IFNγ and TNF.

In some aspects the biological activity is measured by assessing clinical outcome, such as reduction in tumor burden or load. In some aspects, toxic outcomes, persistence and/or expansion of the cells, and/or presence or absence of a host immune response, are assessed. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

### VI. KITS AND ARTICLES OF MANUFACTURE

Also provided are articles of manufacture, systems, apparatuses, and kits useful in performing the provided embodiments. In some embodiments, the provided articles of manufacture or kits contain any of the DNA-targeting systems described herein, any of the gRNAs described herein, any of the fusion proteins described herein, any of the polynucleotides described herein, any of the pluralities of polynucleotides described herein, any of the vectors described herein, any of the pluralities of vectors described herein, any of the cells (e.g. modified T cells) described herein, or a portion or a component of any of the foregoing, or any combination thereof. In some embodiments, the articles of manufacture or kits include polypeptides, polynucleotides, nucleic acids, vectors, and/or cells useful in performing the provided methods.

In some embodiments, the articles of manufacture or kits include one or more containers, typically a plurality of containers, packaging material, and a label or package insert on or associated with the container or containers and/or packaging, generally including instructions for use, e.g., instructions for introducing or administering.

Also provided are articles of manufacture, systems, apparatuses, and kits useful in administering the provided compositions, e.g., pharmaceutical compositions, e.g., for use in therapy or treatment. In some embodiments, the articles of manufacture or kits provided herein contain vectors and/or plurality of vectors, such as any vectors and/or plurality of vectors described herein. In some aspects, the articles of manufacture or kits provided herein can be used for administration of the vectors and/or plurality of vectors, and can include instructions for use.

The articles of manufacture and/or kits containing cells or cell compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

A “gene,” includes a DNA region encoding a gene product. Thus, the gene typically refers to coding and/or transcribed sequences. The sequence of a gene is typically present at a fixed chromosomal position or locus on a chromosome in the cell.

A “regulatory element” or “DNA regulatory element,” which terms are used interchangeably herein, in reference to a gene refers to DNA regions which regulate the production of a gene product, whether or not such regulatory sequences are adjacent to coding and/or transcribed sequences. Accordingly, a regulatory element includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, a “target site” or “target nucleic acid sequence” is a nucleic acid sequence that defines a portion of a nucleic acid to which a binding molecule (e.g. a DNA-binding domain disclosed herein) will bind, provided sufficient conditions for binding exist.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. For instance, expression includes the transcription and/or translation of a particular nucleotide sequence drive by its promoter. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “increased expression” “enhanced expression” or “overexpression” means any form of expression that is additional to the expression in an original or source cell that does not contain the modification for modulating a particular gene expression by a DNA-targeting system, for instance a wild-type expression level (which can be absence of expression or immeasurable expression as well). Reference herein to “increased expression,” “enhanced expression” or “overexpression” is taken to mean an increase in gene expression relative to the level in a cell that does not contain the modification, such as the original source cell prior to contacting with, or engineering to introduce, the Dna-targeting system into the T cell, such as an unmodified cell or a wild-type T cell. The increase in expression can be at least 5%, 10%, 20%, 30%, 40% or 50%, 60%, 70%, 80%, 85%, 90%, or 100% or even more. In some cases, the increase in expression can be at least 2-fold, 5-fold, 10-fold, 20-fold, 30-fold, 40-fold. 50-fold, 60-fold, 70-fold, 80-fold, 90-fold, 100-fold, 200-fold, 300-fold, 400-food, 500-fold, 1000-fold or more.

As used herein, the term “increased transcription” refers to the level of transcription of a gene that is additional to the transcription of the gene in an original or source cell that does not contain the modification for modulating transcription by a DNA-targeting system, for instance a wild-type transcription level of a gene. Reference to increased transcription can refer to an increase in the levels of a transcribable product of a gene such as mRNA. Any of a variety of methods can be used to monitor or quantitate a level of a transcribable product such as mRNA, including but not limited to, real-time quantitative RT (reverse transcriptase)-polymerase chain reaction (qRT-PCR), Northern Blot, microarray analysis, or RNA sequencing (RNA-Seq). The increase in transcription can be at least 5%, 10%, 20%, 30%, 40% or 50%, 60%, 70%, 80%, 85%, 90%, or 100% or even more. In some cases, the increase in transcription can be at least 2-fold, 5-fold, 10-fold, 20-fold, 30-fold, 40-fold, 50-fold, 60-fold, 70-fold, 80-fold, 90-fold, 100-fold, 200-fold or more.

As used herein, an “epigenetic modification” refers to changes in the gene expression that are non-genetic modifications, i.e. not caused by changes in the DNA sequences, but are due to epigenetic changes such as events like DNA methylations or histone modifications. An epigenetic modification may result in a heritable change in gene activity and expression that occur without alteration in DNA sequence. For instance, epigenetic modifications include non-genetic modifications such as chemical modifications to the cytosine residues of DNA (DNA nethylation) and histone proteins associated with DNA (histone modifications).

As used herein, the term “modification” or “modified” with reference to a T cell refers to any change or alteration in a cell that impacts gene expression in the cell. In some embodiments, the modification is an epigenetic modification that directly changes the epigenetic state of a gene or regulatory elements thereof to alter (e.g. increase) expression of a gene product. In some embodiments, a modification described herein results in increased expression of a target gene or selected polynucleotide sequence.

As used herein, a “fusion” molecule is a molecule in which two or more subunit molecules are linked, such as covalently. Examples of a fusion molecule include, but are not limited to, fusion proteins (for example, a fusion between a DNA-binding domain such as a ZFP, TALE DNA-binding domain or CRISPR-Cas protein and one or more effector domains, such as a transactivation domain). The fusion molecule also may be part of a system in which a polynucleotide component associates with a polypeptide component to form a functional system (e.g., a CRISPR/Cas system in which a single guide RNA associates with a functional domain to modulate gene expression). Fusion molecules also include fusion nucleic acids, for example, a nucleic acid encoding the fusion protein. Expression of a fusion protein in a cell can result from delivery of the fusion protein to the cell or by delivery of a polynucleotide encoding the fusion protein to a cell, where the polynucleotide is transcribed, and the transcript is translated, to generate the fusion protein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as adenoviral vectors or lentiviral vectors.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include, but are not limited to, cosmids, plasmids (e.g., naked or contained in liposomes) and viruses (e.g., lentiviruses, retroviruses, adenoviruses, and adeno-associated viruses) that incorporate the recombinant polynucleotide.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomelic “nucleotides.” The monomelic nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, (e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, the term “treating” and “treatment” includes administering to a subject an effective amount of a biological molecule, such as a therapeutic agent, so that the subject has a reduction in at least one symptom of the disease or an improvement in the disease, for example, beneficial or desired clinical results. For instance, a biological molecule may include cells (e.g. T cells), such as cells that have been modified by a DNA-targeting system or polynucleotide(s) encoding the DNA-targeting system described herein. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a biological molecule, such as a compound or cells, that, when administered, is sufficient to prevent development of, or alleviate to some extent, one or more of the signs or symptoms of the disorder or disease being treated. The therapeutically effective amount will vary depending on the biological molecule, the disease and its severity and the age, weight, etc., of the subject to be treated.

As used herein, “adoptive cell therapy” (ACT) refers to the administration of T cells targeting a specific antigen to a subject.

As used herein, the term “autologous” is meant to refer to any material derived from the same individual to which it is later to be re-introduced into the individual.

“Allogeneic” refers to a graft derived from a different animal of the same species.

### VIII. EXAMPLES

The following examples are included for illustrative purposes only and are not intended to limit the scope of the invention.

### Example 1: a CRISPRi Screen for gRNAs Targeting Genes Affecting T-Cell Phenotype

A library of gRNAs targeting genes involved in T cell regulation was screened in a pooled format in primary human T-cells transiently expressing an exemplary dCas9-transcriptional repressor fusion protein to identify gRNAs that upregulate or downregulate cytokines upon T-cell stimulation.

A. Screen of gRNA Library for gRNAs that Regulate Cytokines Via CRISPR-Based Transcriptional Interference (CRISPRi)

A library of approximately 13,500 gRNAs was generated. The library was composed of gRNAs targeted to 77 human genes, with approximately 150 gRNAs per transcription start site (TSS), and approximately 500 control gRNAs with spacers not aligned to the human genome. gRNAs were designed according to the protospacer adjacent motif (PAM) sequence for SpCas9 (5′-NGG-3′). The gRNAs were tiled around the TSS of the 77 target genes, generally within 1 kb and closer to the TSS.

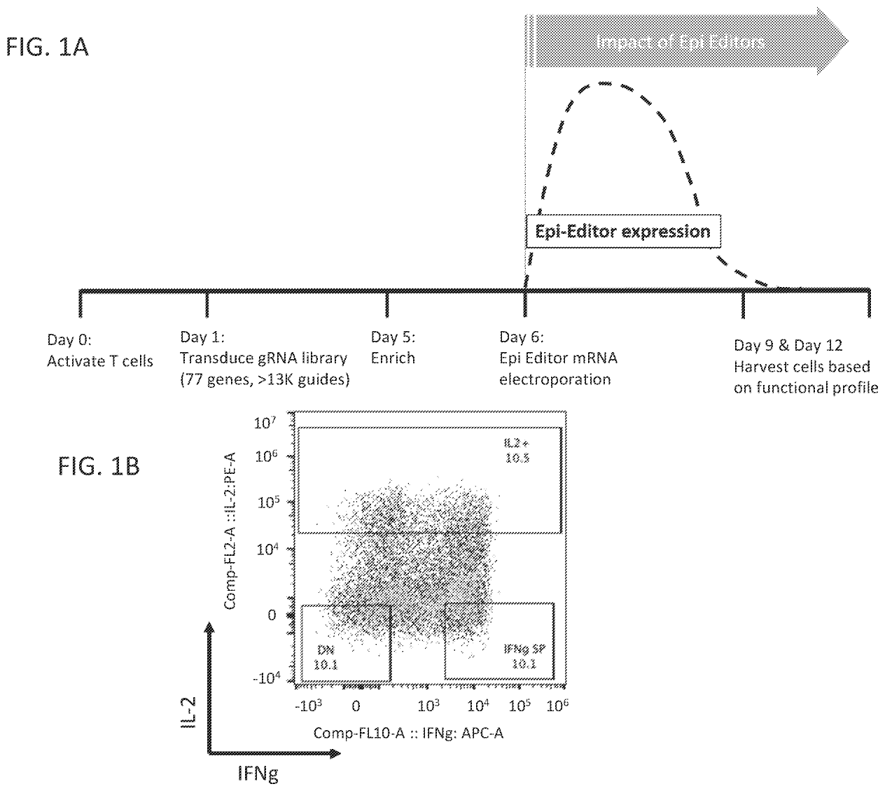

The library was screened to identify gRNAs that upregulate or downregulate cytokine production upon T-cell receptor stimulation in cells transiently expressing the transcriptional repressor domain dSpCas9-KRAB (SEQ ID NO:72, encoding SEQ ID NO:73) or dSpCas9-KRAB-DNMT3A/3L (SEQ ID NO:74, encoding SEQ ID NO:75), two exemplary RNA-guided DNA-targeting nuclease-inactivated Cas9 fusion proteins for transcriptional repression of gRNA-targeted genes. FIG. 1A and FIG. 1B show the screen workflow, which is further described below.

On day 0, primary human CD4+ T cells and CD8+ T cells (at a ratio of 1:1 CD4+ to CD8+ T cells) were activated with an anti-CD3 and anti-CD28 T cell activation reagent (e.g. T cell TransAct™, Miltenyi Biotec). Experiments were performed with two different donors in parallel.

On day 1, 24 hours post stimulation, T cells were transduced with lentiviral constructs encoding the gRNA library. To be able to enrich for gRNA+ cells, the cells were cotransfected with CD90.1 (Thy1.1) and mCherry as reporters. Cells were incubated overnight at 37° C., 5% CO₂, and then fresh media with cytokine was added 24-30 hours after transduction. At days 3-5, transduction efficiency of the gRNA library was monitored based on mCherry fluorescent signal. On day 5, CD90+ cells were enriched by positive selection.

On day 6, gRNA-enriched cells were electroporated with 0.5 μg mRNA encoding dSpCas9-KRAB, or 1 μg mRNA encoding dSpCas9-KRAB-DNMT3A/3L, per 1E6 cells. Cells were then cultured in fresh media until day 8, at which point the cells were stimulated with the anti-CD3/anti-CD28 T cell activation reagent. T cells were harvested for analysis on days 9 or 12. Cells that were harvested on day 12 were restimulated overnight on day 11 with the anti-CD3/anti-CD28 T cell activation reagent prior to their harvest on day 12.

On day 9 or 12, approximately 15 hours after stimulation or restimulation, cells were stained by intracellular cytokine staining (ICS) for IL-2 production and IFN-gamma (IFNg) production. Cells were analyzed by flow cytometry for cells that were positive or negative for IL-2 and IFN-g, and the following populations of cells were collected: (1) an unsorted population, (2) an IL-2+ population, (3) an IFNg+/IL-2-population, and (4) an IL-2−/IFNg-double-negative (DN) population. An exemplary flow cytometry expression plot and sorted populations are shown in FIG. 1B. Screening for an IL-2+ phenotype was chosen as it was expected it may be associated with T cells that are less differentiated, have higher potential for sustained homeostasis, and longevity. Screening for an IFNg+/IL-2-phenotype was chosen as it was expected it may be associated with robust effector T cells with more limited homeostatic potential. gRNAs that facilitate upregulation or downregulation of IL-2 and/or IFNg were expected to be enriched in the respective sorted populations, for example in comparison to the double-negative population. gRNAs that facilitate increased proliferation of transduced cells were expected to be enriched in unsorted cells at day 12 in comparison to day 6 before dCas9 expression. gRNA enrichment was analyzed in order to identify targets whose modulation results in enrichment of cells that express IL-2 and/or IFNg and exhibit increased proliferation, as described below.

To identify enriched gRNAs, genomic DNA was isolated from the populations, amplified, and sequenced. Sequencing was performed to compare the abundance of each gRNA between the respective sorted populations and the unsorted or double-negative population, as described below.

B. Identification of gRNAs and Genes Affecting Proliferation, IL-2 Expression, and/or IFNg Expression.

gRNAs enriched in cells with a negative or positive impact on IL-2 and/or IFN-gamma production, or on proliferation, were identified based on sequencing analysis. gRNA enrichment analysis based on sequencing of sorted cell populations was used to identify gRNAs and corresponding genes that affect proliferation, IL-2 expression, and/or IFN-gamma expression.

gRNAs targeting genes that affect IL-2 and/or IFNg expression were expected to be enriched in the respective FACS-sorted populations, for example in comparison to the double-negative population. For example, a gRNA targeting the transcriptional repressor dCas9-KRAB to a gene whose downregulation leads to upregulation of IL-2 (i.e. a gene that inhibits IL-2 expression) would be expected to be enriched in the IL-2+ population in comparison to the double-negative population. In contrast, a gRNA targeting the transcriptional repressor dCas9-KRAB to a gene whose downregulation leads to downregulation of IL-2 (i.e. a gene that promotes IL-2 expression) would be expected to be depleted from the IL-2+ population in comparison to the double-negative population.

gRNAs targeting genes that affect proliferation were expected to be enriched or depleted from unsorted cells following electroporation with the targeted transcriptional repressor in comparison to unsorted cells pre-electroporation. For example, a gRNA targeting the transcriptional repressor dCas9-KRAB to a gene whose downregulation leads to increased proliferation (i.e. a gene that inhibits proliferation) would be expected to be enriched in unsorted cells at Day 12 (6 days after electroporation) in comparison to Day 6 (before electroporation). In contrast, a gRNA targeting the transcriptional repressor dCas9-KRAB to a gene whose downregulation leads to reduced proliferation (i.e. a gene that promotes proliferation) would be expected to be depleted from unsorted cells at Day 12 (6 days after electroporation) in comparison to Day 6 (before electroporation).

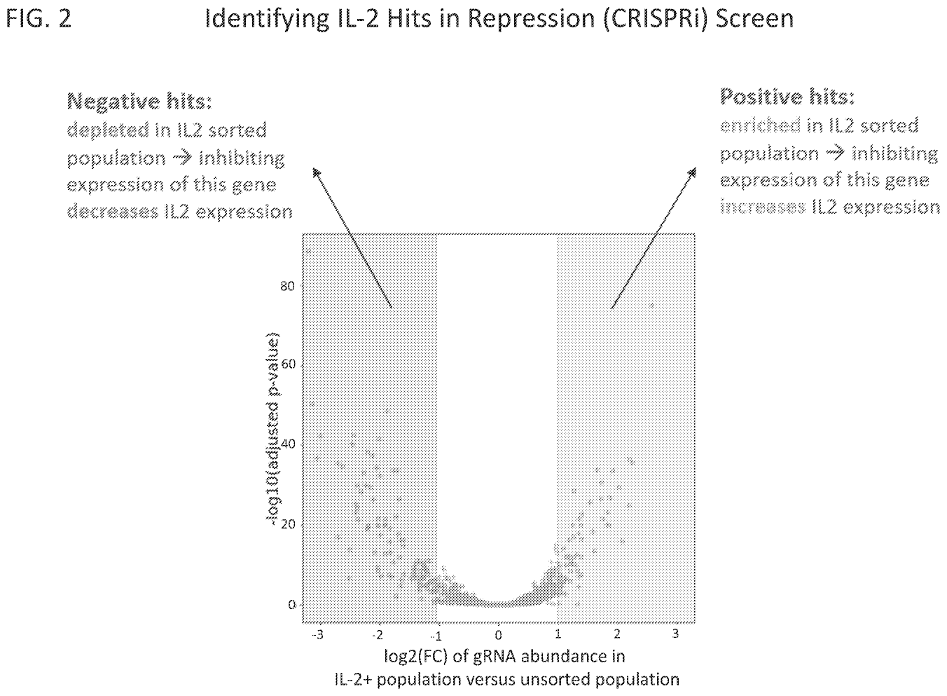

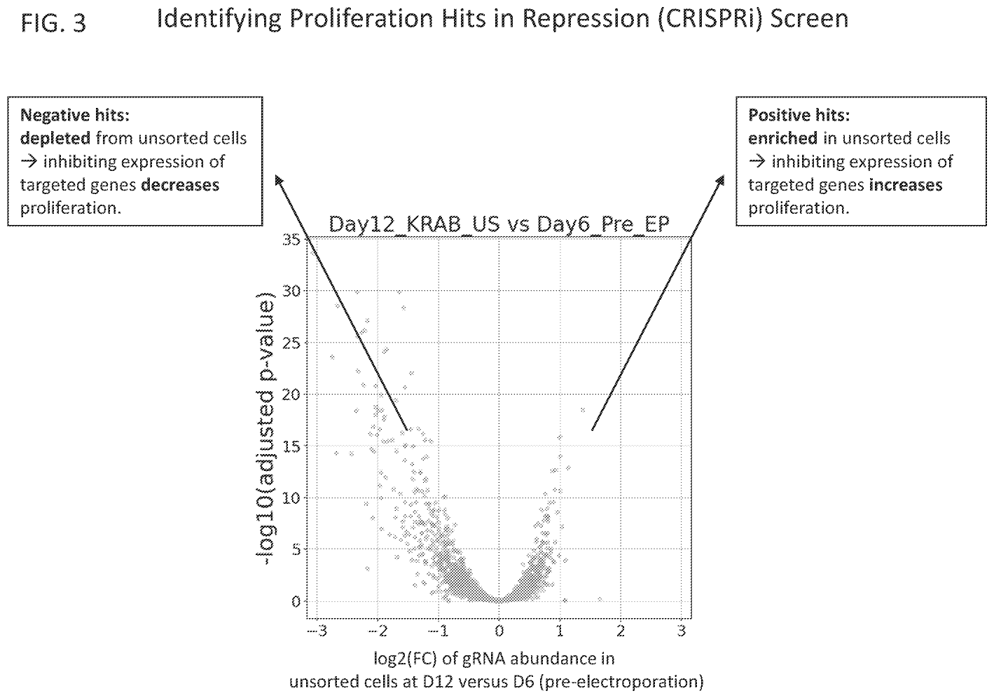

Exemplary results depicted in FIG. 2 show hits for gRNAs targeting genes that, when inhibited, either decrease (negative hits) or increase (positive hits) IL-2 expression as determined using a cutoff with an abs(log 2 FC)>1 and FDR<0.05. None of the non-targeting control gRNAs were identified as a hit. An exemplary non-targeting control gRNA (non-targeting_sp_1) targeted the sequence of SEQ ID NO: 34, and contained the gRNA spacer sequence SEQ ID NO: 68. Exemplary results depicted in FIG. 3 show hits for gRNAs targeting genes that, when inhibited, either decrease (negative hits) or increase (positive hits) proliferation.

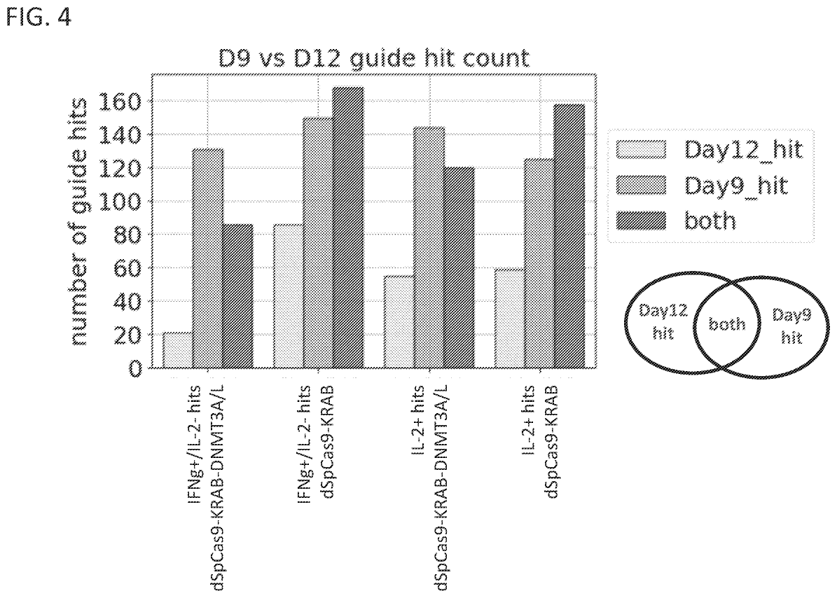

FIG. 4 shows the number of gRNAs that were hits based on similar analysis at day 9 only, day 12 only, or both day 9 and day 12, for the indicated conditions. Fewer hits were obtained at day 12 than at day 9. Without wishing to be bound by theory, it is believed that hits from day 12 represent gRNAs capable of facilitating extended persistence of the relevant phenotype.

Table E1 summarizes gene targets whose repression by CRISPRi upregulated IL-2 and/or IFN-gamma expression in T cells from one or both donors using either transcriptional repressor domain (KRAB or KRAB-DNMT3A/3L). “IL-2 & IFNg hits” shows genes targeted by gRNAs that were enriched in both the IL-2+ population and in the IFNg+/IL-2-population. “IL-2 hits” shows genes targeted by gRNAs that were enriched in the IL-2+ population but not in the IFNg+/IL-2-population. “IFNg hits” shows genes targeted by gRNAs that were enriched in the IFNg+/IL-2-population but not in the IL-2+ population. Enriched gRNAs targeting the genes CD5, RASA2, MYB, CBLB and ELOB (underlined in the table) were also found as proliferation hits (e.g. as shown and described for FIG. 3). Additional exemplary gene targets whose repression by CRISPRi lead to increased proliferation included PRDM1 and CISH (not shown in Table E1). Exemplary gene targets whose repression by CRISPRi downregulated IL-2 and/or IFN-gamma (i.e. negative hits) included VAV1, LAT, LCP2, and CD28 (not shown in Table E1).

TABLE E1
Summary of Repression Screen Gene Hits

| | | |
|---|---|---|
| IL2 & IFNg hits | IL-2 hits | IFNg hits |

| | | | |
|---|---|---|---|
| CD5 | KDM1A | RASA2 | GATA3 |
| | | MYB | ELOB |
| | | CBLB | |
| | | DGKZ | |

Table E2 shows exemplary enriched gRNAs for the gene hits described above. Exemplary designed target site (protospacer) sequences are set forth in SEQ ID NOs: 1-33, 150, and 184-191, and gRNA spacer sequences for gRNAs targeting each site are set forth in SEQ ID NOS: 35-67, 163, and 192-199, as shown in Table E2. Each gRNA further comprised a scaffold sequence for SpCas9, comprising the sequence:

(SEQ ID NO: 69)
GUUUAAGAGCUAUGCUGGAAACAGCAUAGCAAGUUUAAAUAAGGCUAGU
CCGUUAUCAACUUGAAAAAGUGGCACCGAGUCGGUGC.

TABLE E2
Enriched gRNAs from Repression Screen

| | | | gRNA | | Negative |
|---|---|---|---|---|---|
| | Target Site, Protospacer | | spacer | | or |
| | | SEQ ID | SEQ ID | | positive |
| Guide ID | Sequence | NO | NO | Gene | hit |
| CD5_1 | TTGCACTGGAAGGGTAAAGC | 1 | 35 | CD5 | Positive |
| CD5_2 | GGAGGCGACCAAGTAAAGGC | 2 | 36 | CD5 | |
| CD5_3 | GTCAGTGGGGGACCTCGCAG | 3 | 37 | CD5 | |
| KDM1A_1 | CGCGCGGGCAGCGTGAAGCG | 4 | 38 | KDM1A | positive |
| KDM1A_2 | AGCGGCAGCAACCGGGACGG | 5 | 39 | KDM1A | |
| KDM1A_3 | GCCCAGAAGCCCTAAGACCA | 6 | 40 | KDM1A | |
| VAV1_1 | TGTCGCTCCACAGGCGAGCA | 7 | 41 | VAV1 | negative |
| VAV1_2 | CCTCTCAGGGCGACAGTTAC | 8 | 42 | VAV1 | |
| VAV1_3 | GGCCAGCTAGACTATGAGAT | 9 | 43 | VAV1 | |
| CBLB_1 | GAACAGCTCGCTCCCGAAGA | 10 | 44 | CBLB | positive |
| CBLB_2 | CGCTGGGTTGCTCCTTCTTC | 11 | 45 | CBLB | |
| CBLB_3 | CGTCCAGGCAGACGGCGGTG | 12 | 46 | CBLB | |
| DGKZ_1 | GGGACACGGGCGGGATCGGT | 13 | 47 | DGKZ | positive |
| DGKZ_2 | GGAGCGAGCGCGCGCCATGG | 14 | 48 | DGKZ | |
| DGKZ_3 | TCTTCGGGCACAGGTGAGCG | 15 | 49 | DGKZ | |
| MYB_1 | GCCGAATGGGAGCGGCGACC | 16 | 50 | MYB | positive |
| MYB_2 | GGATCCCTCGCCGACACCCG | 17 | 51 | MYB | |
| MYB_3 | GAAACTTCGCCCCAGCGGTG | 18 | 52 | MYB | |
| RASA2_1 | GCACGGGCCGGGCGGCACCA | 19 | 53 | RASA2 | positive |
| RASA2_2 | TGCTGCGGCGGCTTCTTCCG | 20 | 54 | RASA2 | |
| RASA2_3 | GCGCTGGGCGCGAGGCTGAG | 21 | 55 | RASA2 | |
| ELOB_1 | CGAACTCCTTGGGCTAGAAG | 22 | 56 | ELOB | positive |
| ELOB_2 | TGCGGCCGCCATCCCGACGG | 23 | 57 | ELOB | |
| ELOB_3 | CTGGAAGCGGGCGGTATCGA | 24 | 58 | ELOB | |
| GATA3_1 | CGGAGGGTACCTCTGCACCG | 25 | 59 | GATA3 | positive |
| GATA3_2 | TCGACGAGGAGGCTCCACCC | 26 | 60 | GATA3 | |
| GATA3_3 | CAGGGCTGACTGTTACGACT | 27 | 61 | GATA3 | |
| CISH_1 | TGTCCTGCGCCCGCGCGCCC | 28 | 62 | CISH | positive |
| CISH_2 | GGCGGCTGGAGGGAACCAGT | 29 | 63 | CISH | |
| CISH_3 | GTGGCGCGGACCGCCTGCGA | 30 | 64 | CISH | |
| PRDM1_1 | AGAGGCAAGAGCAGCGACCG | 31 | 65 | PRDM1 | positive |
| PRDM1_2 | GACGCGGGGAGAATGTGGAC | 32 | 66 | PRDM1 | |
| PRDM1_3 | TTGCCTCTCCGCAACACTGG | 33 | 67 | PRDM1 | |
| LAT_1 | GGCTGGGACGCAGGGGTAAC | 184 | 192 | LAT | negative |
| LAT_2 | CCACCCCAGGCACTCACCAA | 185 | 193 | LAT | |
| LAT_3 | CTGTGGTGAGCGCCGGGCGA | 186 | 194 | LAT | |
| LCP2_1 | TCCAGTCACCCCAACCCAGT | 150 | 163 | LCP2 | negative |
| LCP2_4 | AAAGACATCGGCTCCAACAG | 187 | 195 | LCP2 | |
| LCP2_5 | AGCCGTTGCTTTCTGGGATC | 188 | 196 | LCP2 | |
| CD28_4 | TCGTCAGGACAAAGATGCTC | 189 | 197 | CD28 | negative |
| CD28_5 | CGTGGATGACGGAGACTCTC | 190 | 198 | CD28 | |
| CD28_6 | CTGAGAGTCTCCGTCATCCA | 191 | 199 | CD28 | |

### Example 2: A CRISPRa Screen for gRNAs Targeting Genes Affecting T-Cell Phenotype

A library of gRNAs targeting genes involved in T cell regulation was screened in a pooled format in primary human T-cells transiently expressing an exemplary dCas9-transcriptional activator fusion protein to identify gRNAs that upregulate or downregulate cytokines upon T-cell stimulation.

The gRNA library described above for Example 1 was screened in a similar format, this time using an exemplary dCas9 fusion protein for transcriptional activation of gRNA-targeted genes, dSpCas9-2xVP64 (SEQ ID NO:76, encoding SEQ ID NO:77).

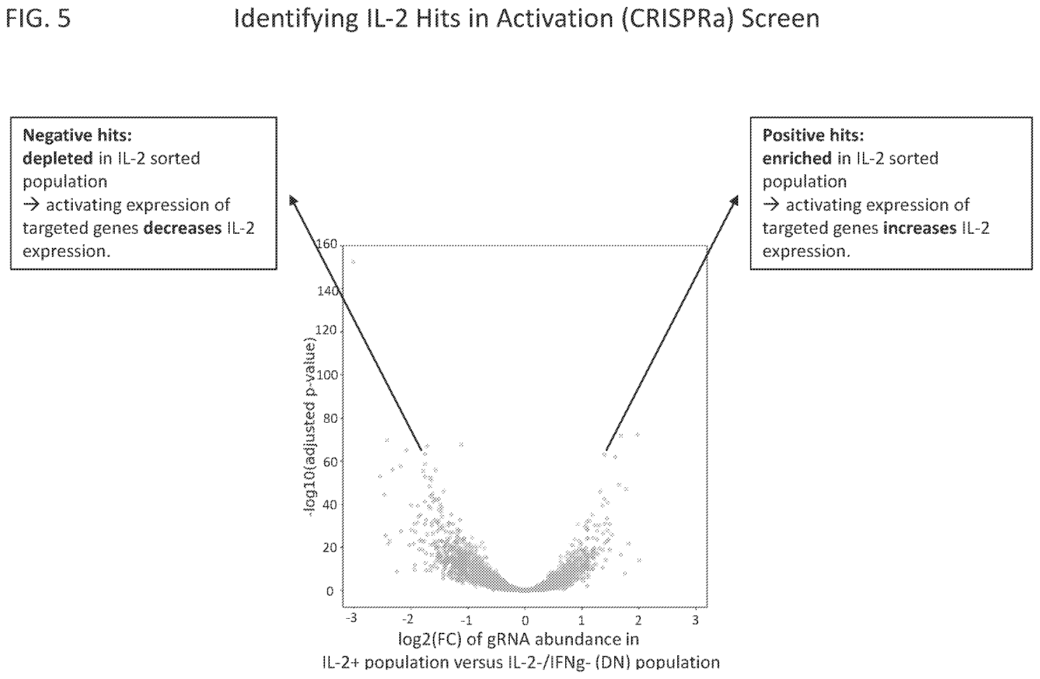

gRNAs enriched in the sorted populations were identified, revealing gRNAs and corresponding target genes that, when transcriptionally activated, promoted the respective phenotypes. FIG. 5 shows an exemplary plot of gRNA abundance in the IL-2+ population versus the IL-2−/IFNg-double negative (DN) population from the activation screen. Positive hits are enriched in the IL-2+ population in comparison to the DN population.

Table E3 shows gene targets whose activation by CRISPRa led to increased IL-2 expression and/or increased IFNg expression, or increased proliferation. Table E4 shows exemplary gRNAs for target genes identified in the activation screen.

TABLE E3
Summary of Activation Screen Gene Hits

| Gene | Phenotype enrichment when activated |
|---|---|
| CD28 | IL2 and IFNg |
| LCP2 | IL2 and IFNg |
| TBX21 | IL2 and IFNg |
| VAV1 | IL2 and IFNg |
| EOMES | IFNg |
| BATF | Proliferation |
| IRF4 | Proliferation |

TABLE E4
Exemplary gRNAs for target genes identified in activation screen

| | | Target Site/ | | |
|---|---|---|---|---|
| | Target Site/ | Protospacer | gRNA spacer | |
| Guide ID | Protospacer Sequence | SEQ ID NO | SEQ ID NO | Gene |
| CD28_1 | CTGACTGCAGCATTTCACAC | 144 | 157 | CD28 |
| CD28_2 | GCTGCAGTCAGGATGCCTTG | 145 | 158 | CD28 |
| CD28_3 | CCTTGATCATGTGCCCTAAG | 146 | 159 | CD28 |
| EOMES_1 | CGCAGTAGCGGCCCGCGAGT | 147 | 160 | EOMES |
| EOMES_2 | CGGGCCGCTACTGCGCGTAC | 148 | 161 | EOMES |
| EOMES_3 | GCTACCCACCTGCCGACTCG | 149 | 162 | EOMES |
| LCP2_1 | TCCAGTCACCCCAACCCAGT | 150 | 163 | LCP2 |
| LCP2_2 | GGTGCTACACTGTGCAGACA | 151 | 164 | LCP2 |
| LCP2_3 | CAGGGTTGGTAATTCCCCAC | 152 | 165 | LCP2 |
| TBX21_1 | CAGGGCCGAGGTGGCGGAGT | 153 | 166 | TBX21 |
| TBX21_2 | CTCGCTTCTCTCCACCATGG | 154 | 167 | TBX21 |
| TBX21_3 | CTAGGCAAATTCTACGCTCT | 155 | 168 | TBX21 |
| VAV1_4 | CCTGTAACTGTCGCCCTGAG | 156 | 169 | VAV1 |
| VAV1_2 | CCTCTCAGGGCGACAGTTAC | 8 | 42 | VAV1 |
| BATF_1 | ACTCACGCTGGAAGTCACAT | 172 | 178 | BATF |
| BATF_2 | AAGTCCGTCTTCTGTCAACA | 173 | 179 | BATF |
| BATF_3 | GCAGAGGGACTGCTCCCCAA | 174 | 180 | BATF |
| IRF4_1 | CCGCTTCGGGGACTGTCACT | 175 | 181 | IRF4 |
| IRF4_2 | ACTTTGCAAGCCGAGAGCCG | 176 | 182 | IRF4 |
| IRF4_3 | GTCCAACCCCCGGCCCCCAC | 177 | 183 | IRF4 |

### Example 3: Identification of Additional Genes and gRNAs for Modulating T Cell Phenotypes

Additional genes and gRNAs were identified for modulating T cell phenotypes.

Additional gRNAs were designed for targeted repression ofgenes including MED12, CCNC, FAS, TGFBR2, and Fli1, as shown in Table E5.A. Additional gRNAs were designed for targeted activation of genes including VAV1 and IL-2, as shown in Table E5.1B.

TABLE E5.A
gRNAs for targeted gene repression for modulating T cell phenotypes

| | | | gRNA | | |
|---|---|---|---|---|---|
| | Target Site/Protospacer | | spacer | | |
| | | SEQ ID | SEQ | | Activation or |
| Guide ID | Sequence | NO | ID NO | Gene | Repression |
| Med12_1 | ACCATTGCCGGAAACTACCG | 80 | 91 | MED12 | Repression |
| Med12_2 | GTGCCCCGGGAGTTTTTCGG | 81 | 92 | MED12 | Repression |
| Med12_3 | ACGGCGGCCGAGAGACAACA | 82 | 93 | MED12 | Repression |
| Med12_4 | CGAGGTACGCCGGGAACCAT | 83 | 94 | MED12 | Repression |
| Med12_5 | CGCCACCGCCGAAAAACTCC | 84 | 95 | MED12 | Repression |
| Med12_6 | CGATGGTTCCCGGCGTACCT | 85 | 96 | MED12 | Repression |
| Med12_7 | CGGCGGCCGAGAGACAACAA | 86 | 97 | MED12 | Repression |
| Med12_8 | TCCTGAGGGTAAACATCGGG | 87 | 98 | MED12 | Repression |
| Med12_9 | TTCGTAGCTCAAGATCCCGA | 88 | 99 | MED12 | Repression |
| Med12_10 | GCTGACTGGGGGAACGGGAA | 89 | 100 | MED12 | Repression |
| Med12_11 | GGCTGGTGCCTCCGGCGCTA | 90 | 101 | MED12 | Repression |
| CCNC_1 | AAAGTTCCGGCCCGCGGTAG | 102 | 113 | CCNC | Repression |
| CCNC_2 | GGGCCGGAACTTTTGTCGAT | 103 | 114 | CCNC | Repression |
| CCNC_3 | CGACGGCGAAAGGAAGAGGA | 104 | 115 | CCNC | Repression |
| CCNC_4 | CGAGGAGCGCGGTTACCGGA | 105 | 116 | CCNC | Repression |
| CCNC_5 | CGGCCGGCGTGAAGGAGACT | 106 | 117 | CCNC | Repression |
| CCNC_6 | TCACGAGAGCTCGCGGCGGT | 107 | 118 | CCNC | Repression |
| CCNC_7 | CTGGGTCTATGGTCGCTCCG | 108 | 119 | CCNC | Repression |
| CCNC_8 | GAACTTTTGTCGATAGGAAC | 109 | 120 | CCNC | Repression |
| CCNC_9 | GCTGATTTGATCGAGGAGCG | 110 | 121 | CCNC | Repression |
| CCNC_10 | GGAGGAGCGCGGTTACCGGA | 111 | 122 | CCNC | Repression |
| CCNC_11 | GTGGGTCTATGGTCGCTCCG | 112 | 123 | CCNC | Repression |
| FAS_1 | GACCCGCTCAGTACGGAGTT | 200 | 212 | FAS | Repression |
| FAS_2 | TCCCCAACTCCGTACTGAGC | 201 | 213 | FAS | Repression |
| FAS_3 | GTTGGTGGACCCGCTCAGTA | 202 | 214 | FAS | Repression |
| FAS_4 | GGACCCGCTCAGTACGGAGT | 203 | 215 | FAS | Repression |
| FAS_5 | ACCCGCTCAGTACGGAGTTG | 204 | 216 | FAS | Repression |
| FAS_6 | GAAGCAGTGGTTAAGCCGGA | 205 | 217 | FAS | Repression |
| FAS_7 | TTCCCCAACTCCGTACTGAG | 292 | 296 | FAS | Repression |
| FAS_8 | GGGAAGCTCTTTCACTTCGG | 293 | 297 | FAS | Repression |
| FAS_9 | ACTGTAAGTCGCTGCCTGAGTGG | 294 | 298 | FAS | Repression |
| FAS_10 | GAGCGGGTCCACCAACCCGC | 295 | 299 | FAS | Repression |
| Fli1_1 | CGCGCGGCGGCCCAGGAGGG | 206 | 218 | Fli1 | Repression |
| Fli1_2 | GCGCTCGCAGGGGGCACGCA | 207 | 219 | Fli1 | Repression |
| Fli1_3 | ACAACAACAAACGTGCACAG | 208 | 220 | Fli1 | Repression |
| Fli1_4 | GAGGGCAGGGCGCTCGCAGG | 209 | 221 | Fli1 | Repression |
| Fli1_5 | AAACGTGCACAGGGGAGTGA | 210 | 222 | Fli1 | Repression |
| Fli1_6 | GAGCGAAAGAGACAGTTAAC | 211 | 223 | Fli1 | Repression |
| TGFBR2_1 | ACTTCAACTCAGCGCTGCGG | 300 | 303 | TGFBR2 | Repression |
| TGFBR2_2 | AGTCCGGCTCCTGTCCCGAG | 301 | 304 | TGFBR2 | Repression |
| TGFBR2_3 | GAAACTCCTCGCCAACAGCT | 302 | 305 | TGFBR2 | Repression |
| TGFBR2_4 | GTCCCGAGCGGGTGCACGCG | 306 | 309 | TGFBR2 | Repression |
| TGFBR2_5 | GTCCGGCTCCTGTCCCGAGC | 307 | 310 | TGFBR2 | Repression |
| TGFBR2_6 | CCCGAGCGGGTGCACGCGCG | 308 | 311 | TGFBR2 | Repression |

TABLE E5.B
gRNAs for targeted gene activation for modulating T cell phenotypes

| | | | gRNA | | |
|---|---|---|---|---|---|
| | Target Site / Protospacer | | spacer | | |
| | | SEQ ID | SEQ ID | | Activation or |
| Guide ID | Sequence | NO | NO | Gene | Repression |
| IL-2_1 | GAGAGCTATCACCTAAGTGT | 78 | 79 | IL-2 | Activation |
| VAV1_5 | CCAGGCCTGTGTCGAGTGGG | 170 | 171 | VAV1 | Activation |

### Example 4: Validation of gRNAs for Epigenetic Repression of Target Genes by CRISPRi

DNA-targeting systems composed of an exemplary dCas9-effector fusion protein for transcriptional repression and guide RNAs (gRNAs) were tested for ability to repress expression of target genes including MED12, CCNC, and FAS in T cells.

Multiple gRNAs targeting the genes were designed by selecting target sites according to the protospacer adjacent motif (PAM) sequence for SpCas9 (5′-NGG-3′). Exemplary designed target site (protospacer) sequences for MED12 are set forth in SEQ ID NOs: 80-90 and gRNA spacer sequences for gRNAs targeting each site are set forth in SEQ ID NOS: 91-101 (Table E5.A). Exemplary designed target site (protospacer) sequences for CCNC are set forth in SEQ ID NOs: 102-112 and gRNA spacer sequences for gRNAs targeting each site are set forth in SEQ ID NOS: 113-123 (Table E5.A). Exemplary designed target site (protospacer) sequences for FAS are set forth in SEQ ID NOs:200-205 and 292-295 and gRNA spacer sequences for gRNAs targeting each site are set forth in SEQ ID NOS:212-217 and 296-299. Each gRNA further comprised a scaffold sequence for SpCas9, comprising the sequence:

(SEQ ID NO: 69)
GUUUAAGAGCUAUGCUGGAAACAGCAUAGCAAGUUUAAAUAAGGCUAGU
CCGUUAUCAACUUGAAAAAGUGGCACCGAGUCGGUGC.

Human primary T cells from two different donors were transiently transfected with the generated gRNAs and dSpCas9-KRAB (SEQ ID NO:72, encoding SEQ ID NO:73), and assessed for knockdown of the targeted genes.

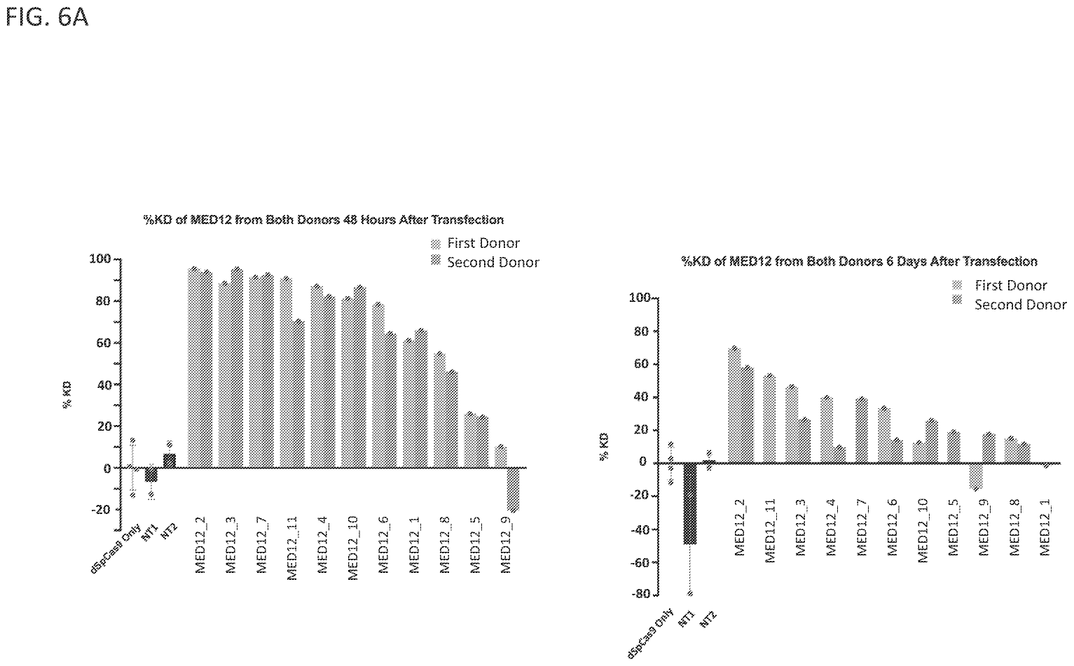

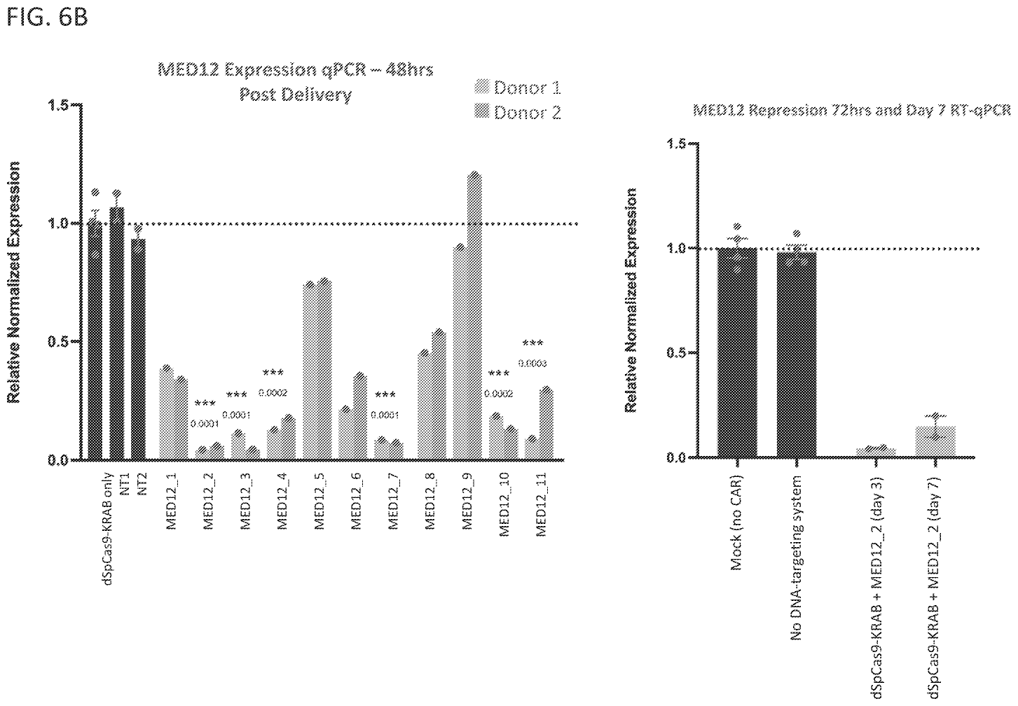

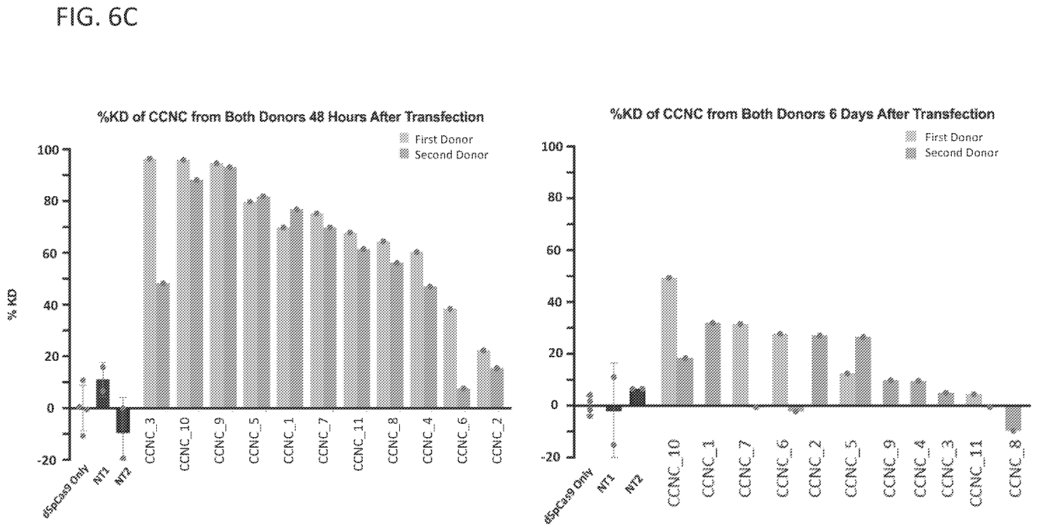

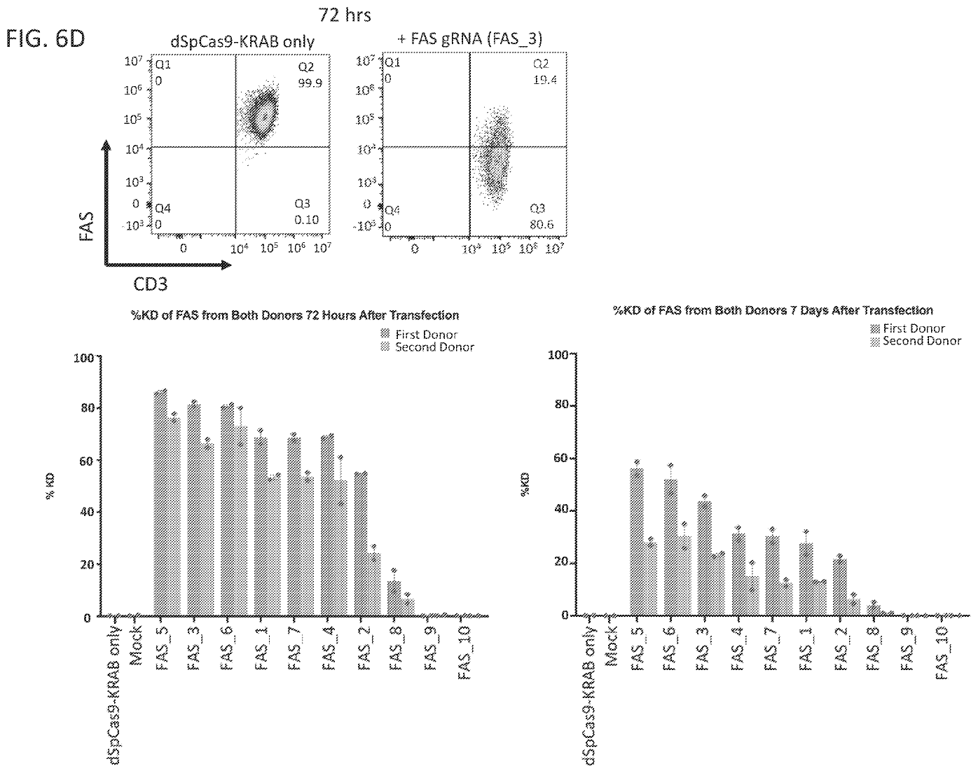

For MED12, mRNA was isolated from the transfected cells at 48 hours, 72 hours, 6 days, and 7 days after transfection, and assessed for knockdown of the target gene by qRT-PCR. Exemplary qRT-PCR results for MED12 are depicted in FIG. 6A-6B, which show that expression of dSpCas9-KRAB and the indicated MED12-targeting gRNAs led to transcriptional repression of MED12 to varying levels, with some gRNAs demonstrating 90% or greater knock down at 48 hours. Several gRNAs facilitated sustained knockdown at 6 days after transfection. As shown in FIG. 6B, delivery of dSpCas9-KRAB and the gRNA MED12_2 (targeting SEQ ID NO:81) led to robust and sustained knockdown of MED12 for at least 7 days following delivery.

For CCNC, mRNA was isolated from the transfected cells at 48 hours and 6 days after transfection, and assessed for knockdown of the target gene by qRT-PCR. Exemplary qRT-PCR results for CCNC are depicted in FIG. 6C, which shows that expression of dSpCas9-KRAB and the indicated CCNC-targeting gRNAs led to transcriptional repression of CCNC to varying levels, with some gRNAs demonstrating sustained knockdown of CCNC at 6 days after transfection.

To assess FAS knockdown, cells were assessed by flow cytometry for expression of FAS at 72 hours and 7 days after transduction. Exemplary results for FAS are shown in FIG. 6D, which shows that expression of dSpCas9-KRAB and the indicated FAS gRNAs led to transcriptional repression of FAS to varying levels. Several gRNAs facilitated knockdown at both 72 hours and 7 days after transfection.

### Example 5: Modulation of T Cell Phenotypes with DNA-Targeting Systems

DNA-targeting systems for epigenetic modulation of target genes containing gRNAs identified in the preceding Examples and dCas9 effector fusion proteins for repression or activation were tested for ability to modulate phenotypes in CAR T cells.

For CAR T cell experiments, CD4 and CD8 T cells from healthy donors were thawed and activated on day 0 with the anti-CD3/anti-CD28 T cell activation reagents as described above, and transduced with a polynucleotide encoding a chimeric antigen receptor (CAR) 24 hours after CD3/CD28 activation (day 1). CAR T cells were derived from various donors, and a referenced donor, first donor, or second donor in any given experiment or corresponding figure does not necessarily correspond to the same donor, first donor, or second donor from another experiment or figure. In these experiments, the polynucleotide encoded an exemplary Her2-targeted chimeric antigen receptor (Her2 CAR), although any other suitable CARs could be used. Further, in this experiment, the polynucleotide encoding the CAR also included a truncated EGFR (EGFRt) marker as a surrogate marker for T cells expressing the CAR (i.e. Her2 CAR T cells). Her2 CAR T cells were electroporated, generally on day 4, with mRNA encoding the dCas9 effector fusion protein and pre-transcribed gRNAs to achieve transient expression of the DNA-targeting systems. For transcriptional repression, experiments were carried out with dSpCas9-KRAB (SEQ ID NO:72, encoding SEQ ID NO:73) or dSpCas9-KRAB-DNMT3A/L (SEQ ID NO:74, encoding SEQ ID NO:75). For transcriptional activation, experiments were carried out with dSpCas9-2xVP64 (SEQ ID NO:76, encoding SEQ ID NO:77). In some cases, Her2 CAR T cells were expanded until day 9 and cryopreserved prior to thawing and further functional characterization. Her2 CAR T cells were stimulated by incubation with Her2 antigen-expressing tumor cell lines (i.e. target cells) such as SKOV3, 143B (ATCC CRL-8303), or NCI-H1975 (ATCC CRL-5908) at various time points and CAR T cell:target cell ratios to mimic physiological conditions. In some experiments, Her2 CAR T cells underwent multiple rounds of stimulation (i.e. serial stimulation) by co-culture with target cells. Serial stimulations were performed by replating CAR T cells with fresh target cells every three to four days, at ratios indicated in the Examples below, generally using a ratio of approximately 1:4 CAR T cells:target cells, depending on the experiment. DNA-targeting systems were delivered only once (generally at day 4, as described above), and not re-delivered during re-stimulations. Experiments where data was collected after serial stimulations are indicated in the text and figures.

Resulting phenotypes of the Her2 CAR T cells were assessed based on multiple criteria, including cytokine expression, proliferation, and ability to kill target cells, as described below.

For intracellular cytokine staining (ICS), cells were labeled with antibodies for cytokines (including for IL-2, IFNg, and TNF-alpha (TNFa)), and assessed for intracellular expression of the cytokines by flow cytometry.

Secreted cytokines were measured in the cell culture medium, using immunoassays from Meso Scale Discovery (MSD) (e.g. as described at www.mesoscale.con/en/technical_resources/our_technology/our_immunoassays). MSD immunoassays combine electrochemiluminescence and multiarray technology for detection of multiple proteins in a single sample. MSD Immunoassays are sandwich-based and involve a Multi-Spot microplate, where each spot is coated with unique capture antibodies.

To measure proliferation (i.e. fold expansion), CAR T cell numbers were determined and compared before and after stimulations with target cells. To count CAR T cells in co-culture with target cells, total cell counts from the co-culture were multiplied by the proportion of total cells that were CAR T cells (e.g. multiplied by 0.5 if the proportion of total cells that were CAR T cells was 50%), based on expression of the transgenic EGFRt marker as assessed flow cytometry using an anti-EGFR antibody.

To measure ability of CAR T cells to kill target cells (i.e. cytotoxic activity), numbers of target cells were tracked in co-culture with the CAR T cells using an Incucyte automated imaging system. Her2 antigen-expressing target cells expressed a fluorescent tag (Nuclight Red), allowing imaging and quantification of target cell numbers over time to generate a growth curve for the target cells. Reduced fluorescence over time was indicative of decreased target cell numbers, and increased killing. The area under the growth curve was used to calculate a killing index to compare conditions, with a higher killing index reflecting a lower area under the curve and greater killing (i.e. cytotoxic activity) by the CAR T cells.

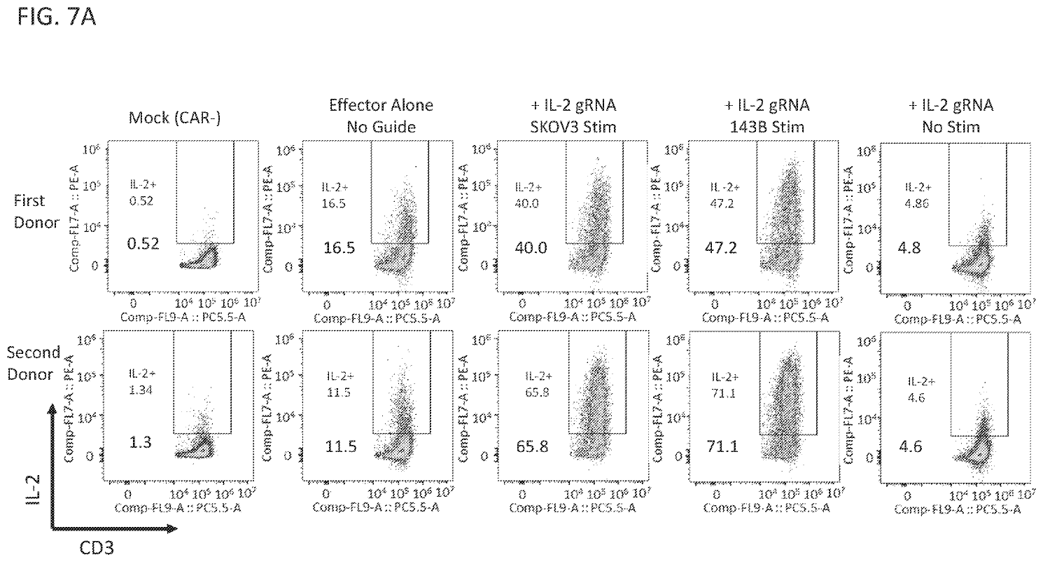

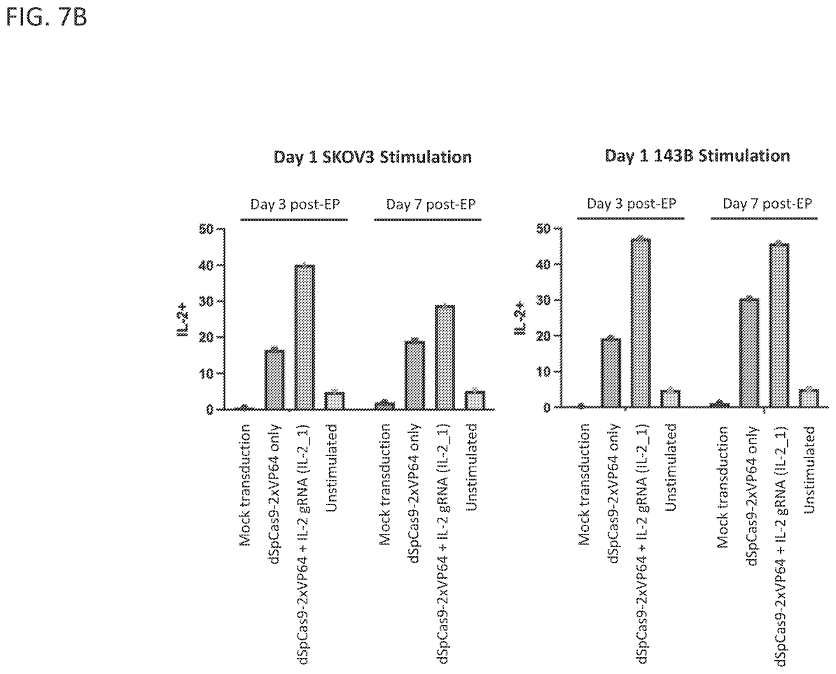

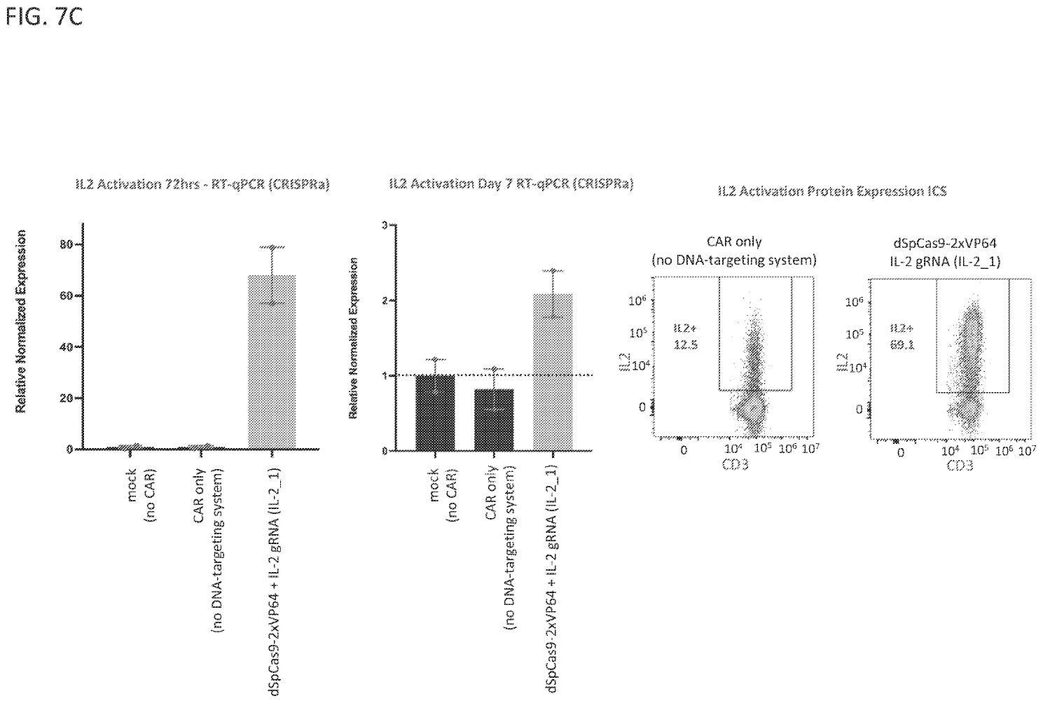

As shown in FIG. 7A, activation of IL-2 with a transiently expressed DNA-targeting system containing dSpCas9-2xVP64 and the IL-2-targeting gRNA targeting SEQ ID NO:78 (IL-2_1) led to increased IL-2 cytokine production (as assessed by ICS and flow cytometry) in stimulated Her2 CAR T cells from two different donors at day 3 post-electroporation (EP). The Her2 CAR T cells were stimulated with Her2 antigen-expressing cells. IL-2 expression was increased in comparison to control cells lacking the CAR (mock), lacking the gRNA (effector alone), or lacking stimulation with a Her2-expressing cell line (no stim). IL-2 expression was increased in CAR T cells from both donors, and in CAR T cells stimulated with either of two different target cell lines (SKOV3 or 143B). As shown in FIG. 7B, the DNA-targeting system for IL-2 activation led to increased IL-2 expression (shown as percentage of cells positive for IL-2) in comparison to control cells at both day 3 and day 7 post-EP.

Her2 CAR T cells were electroporated with a transiently expressed DNA-targeting system for IL-2 activation containing dSpCas9-2xVP64 and the IL-2-targeting gRNA targeting SEQ ID NO:78 (IL-2_1), and assessed for IL-2 mRNA expression levels by RT-qPCR at day 3 and day 7 post-EP. Control cells included cells lacking the CAR (mock), and cells not delivered with the DNA-targeting system (CAR only). IL-2 mRNA expression levels were normalized to the mock control cells. As shown in FIG. 7C, electroporation of the DNA-targeting system for IL-2 activation increased IL-2 mRNA expression levels by greater than 60-fold at 3 days (left panel), and greater than 2-fold at 7 days (middle panel) post-EP. At 7 days post-EP, CAR T cells were stimulated with Her2 antigen-expressing tumor cells (NCI H1975), and the cells were further assessed by ICS and flow cytometry for IL-2 protein expression one day later. As shown in FIG. 7C (right panel), cells delivered with the DNA-targeting system for IL-2 activation exhibited increased expression of IL-2 as compared to CAR T cells not delivered with the DNA-targeting system.

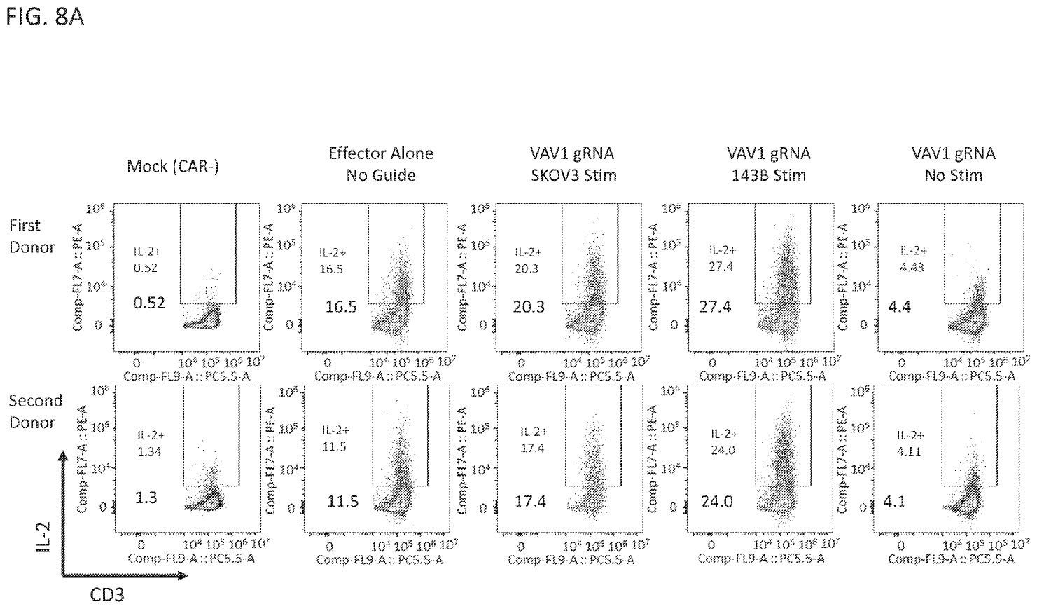

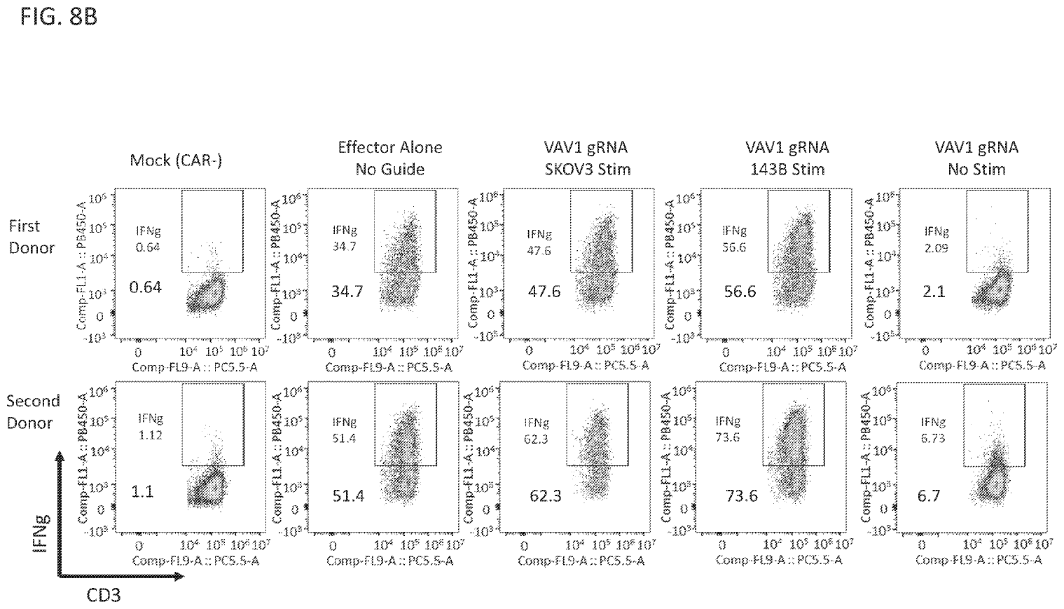

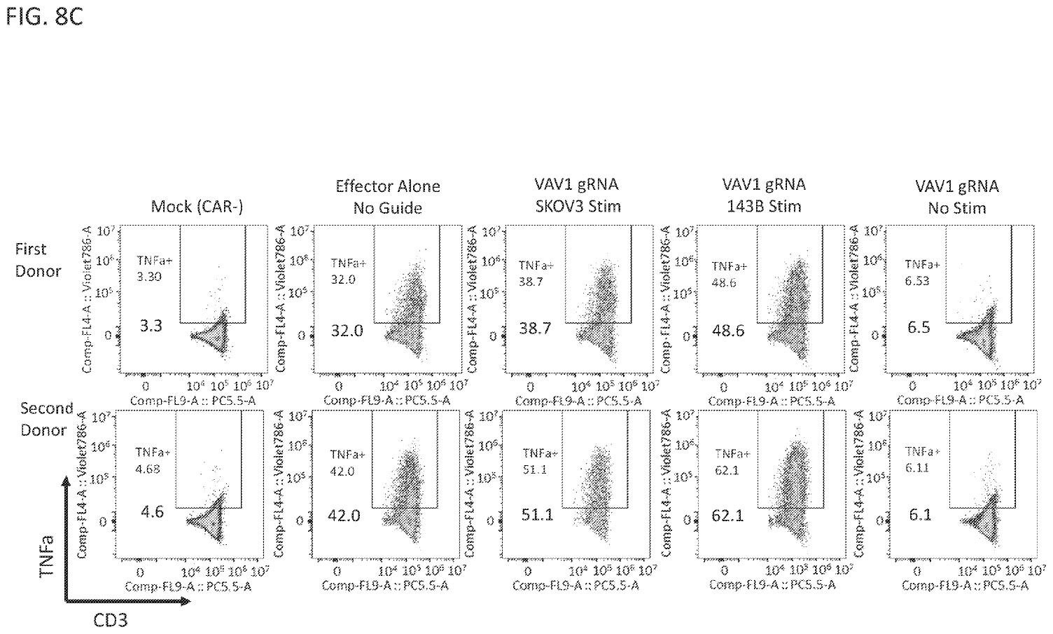

As shown in FIGS. 8A-8C, activation of VAV1 using a transiently expressed DNA-targeting system containing dSpCas9-2xVP64 and the VAV1-targeting gRNA targeting SEQ ID NO:170 (VAV1_5) led to increased IL-2 (FIG. 8A), IFNg (FIG. 8B), and TNFa (FIG. 8C) intracellular cytokine production (as assessed by ICS and flow cytometry) in stimulated Her2 CAR T cells, as compared to control cells.

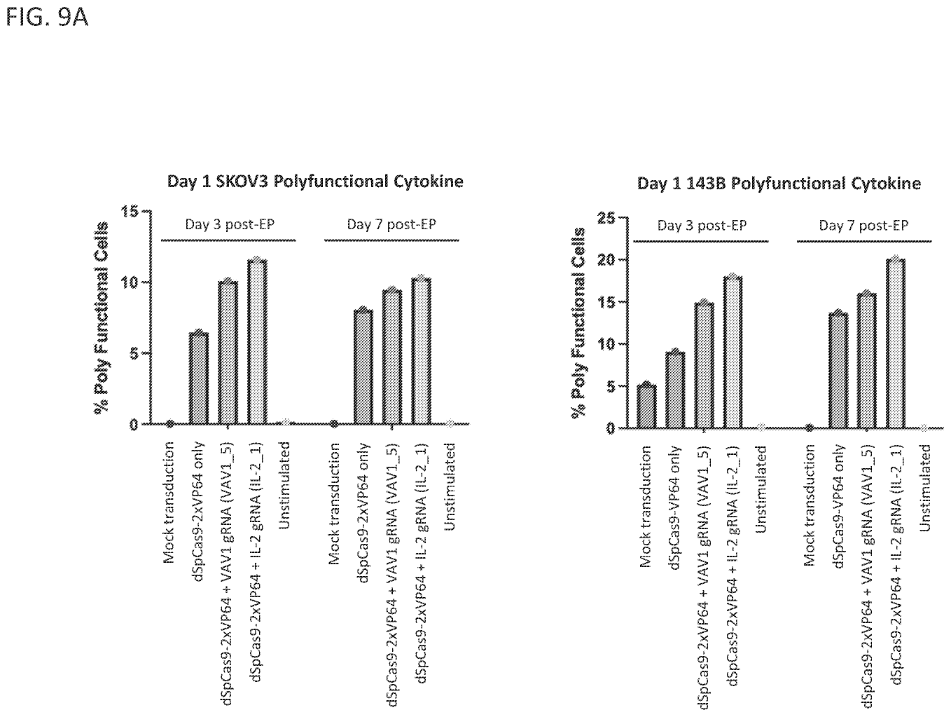

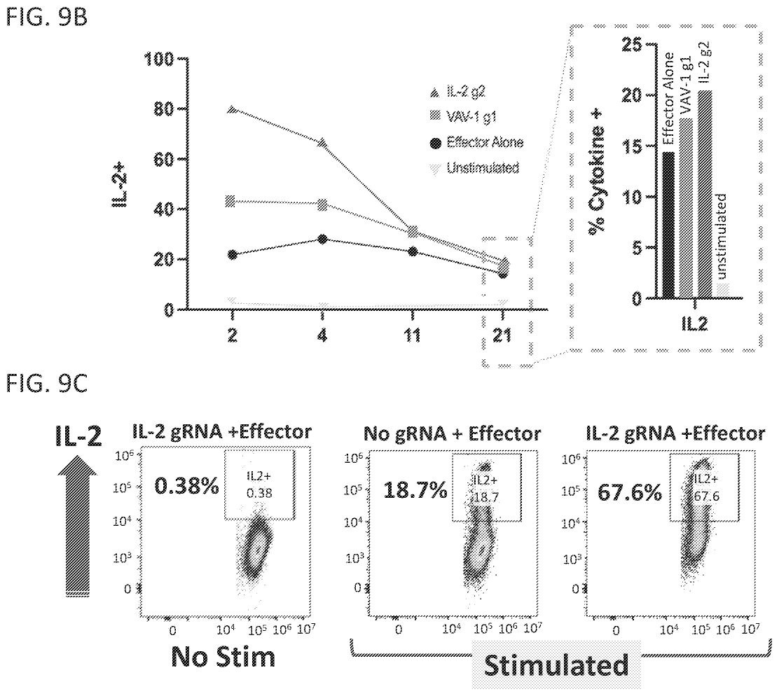

As shown in FIG. 9A, activation of IL-2 or VAV1 using a transiently expressed DNA-targeting system containing dSpCas9-2xVP64 and gRNA IL-2_1 or gRNA VAV1_5, respectively, led to an increase in the percentage of polyfunctional T cells (i.e. triple-positive IL-2+/IFNg+/TNFa+ T cells) at both day 3 and day 7 post-electroporation (post-EP), in comparison to control cells (as assessed by ICS and flow cytometry).

As shown in FIG. 9B, activation of IL-2 or VAV1 using a transiently expressed DNA-targeting system containing effector dSpCas9-2xVP64 and gRNA IL-2_2 or gRNA VAV1_1, respectively, led to an increase in the cytokine output for over the days of monitoring, but then reverted back to regulation by natural components, preventing overactivity.

Further, as shown in FIG. 9C the increase in IL-2 by the DNA-binding systems requires stimulation of the cells as shown for the exemplary DNA-targeting system targeting IL-2 activation in combination with the effector fusion dSpCas9-2xVP64, which shows increase in the percentage of IL-2 expression post stimulation.

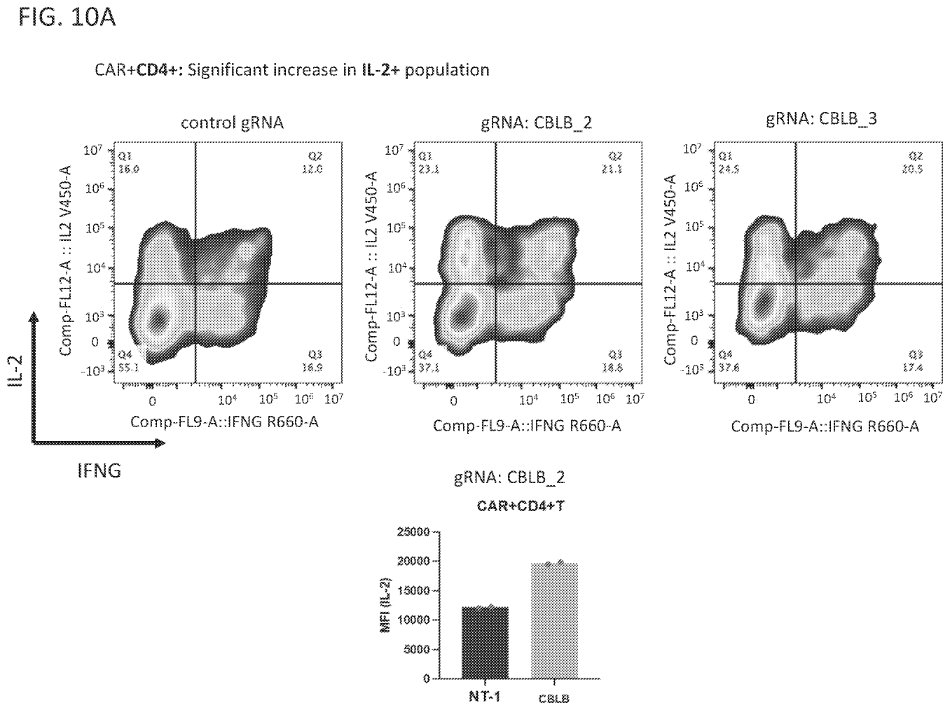

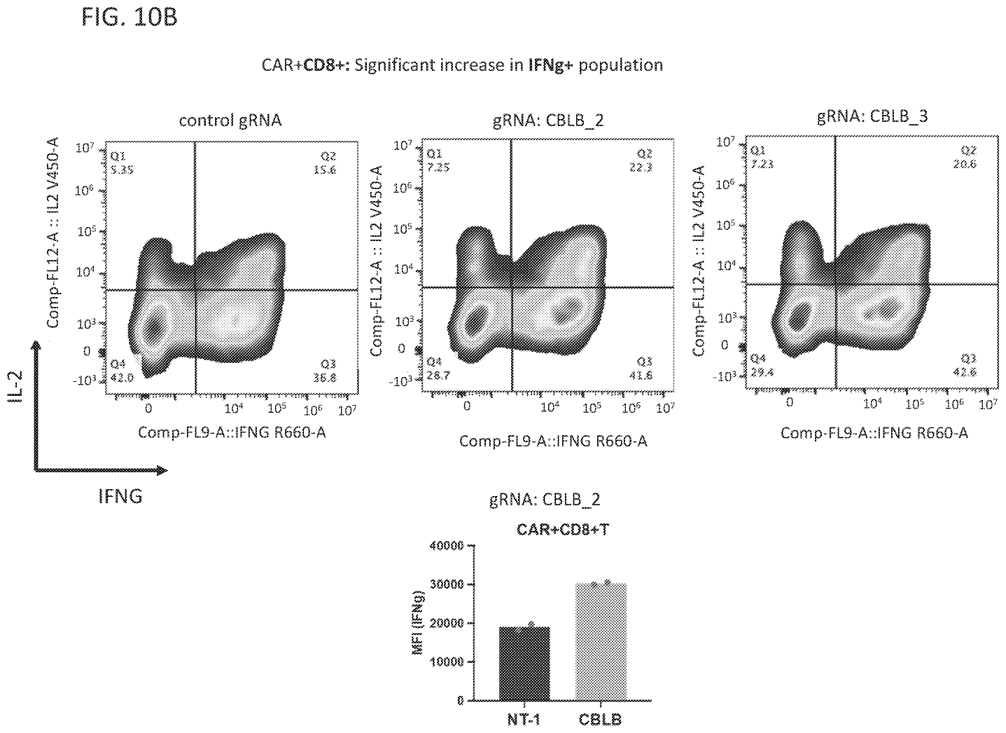

As shown in FIGS. 10A-10B, repression of CBLB using a transiently expressed DNA-targeting system containing dCas9-KRAB and the indicated CBLB-targeting gRNAs (gRNA CBLB_2, targeting SEQ ID NO:11 or gRNA CBLB_3, targeting SEQ ID NO:12) led to increased IL-2 expression in CD4+ CAR T cells, and increased IFNg expression in CD8+ CAR T cells, in comparison to control CAR T cells electroporated with a non-targeting gRNA (as assessed by ICS and flow cytometry). Expression was increased based on both the percentage of cells quantified as positive, and by mean fluorescence intensity (MFI) of the indicated cytokines, as assessed by FACS.

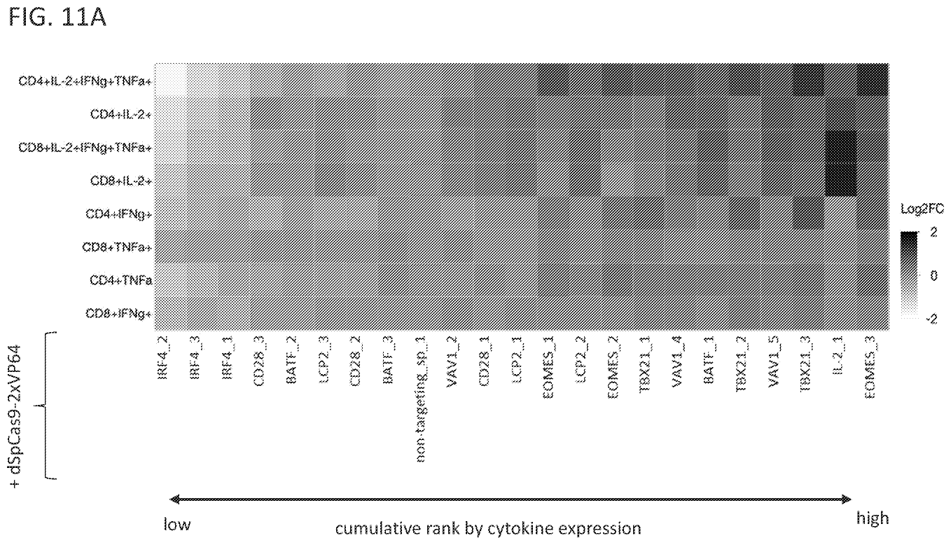

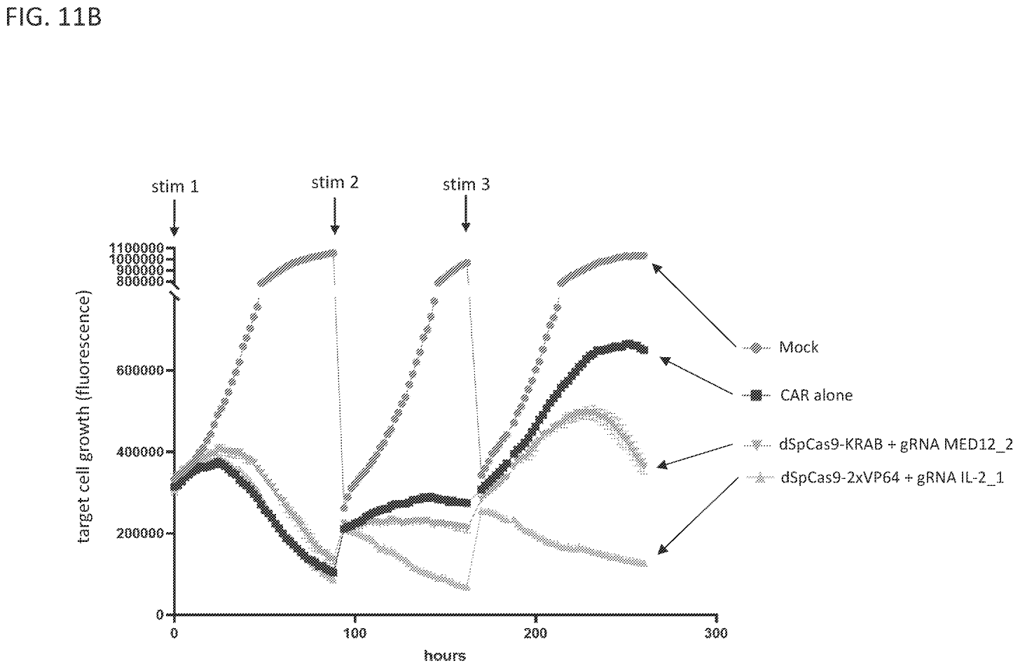

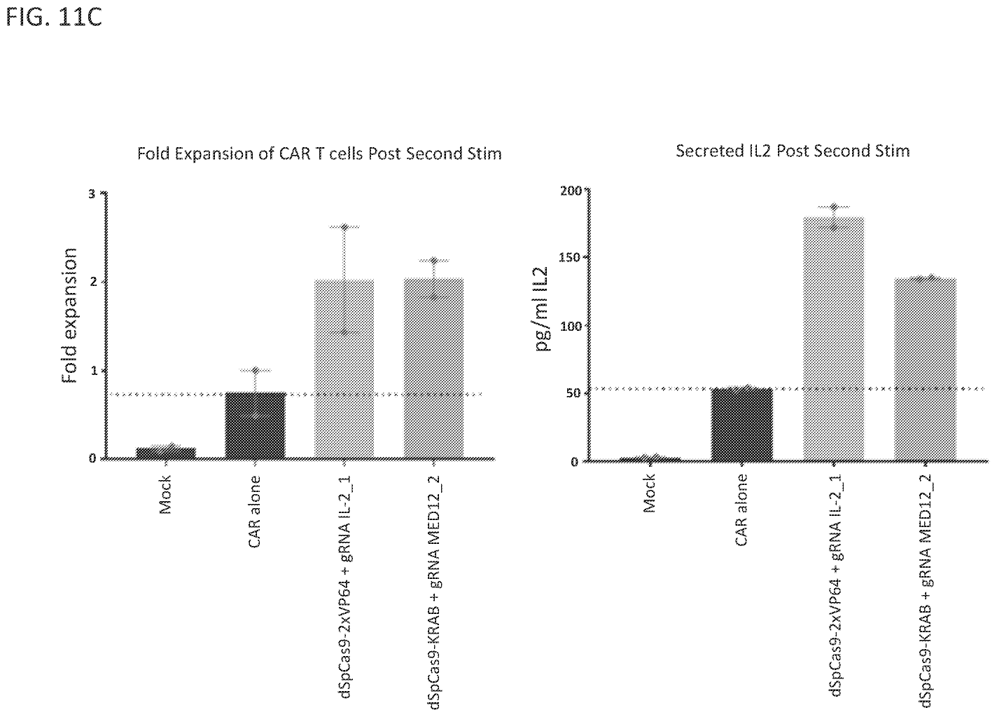

As shown in FIG. 11A, DNA-targeting systems containing dSpCas9-2xVP64 and gRNAs for activation of target genes identified in the preceding Examples and as shown in Table E4 and Table E5.B were tested to identify systems that promote advantageous phenotypes in CAR T cells. DNA-targeting systems containing dSpCas9-2xVP64 and the individual gRNAs were electroporated into Her2 CAR T cells for transient expression, and the Her2 CAR T cells were stimulated by co-culture with the Her2 expressing tumor cell line, 143B. Each condition was assessed (by ICS) based on the percentage of cells with specific intracellular cytokine expression profiles. Cytokine expression was quantified according to % Her2 CAR T cells with the indicated phenotypes (e.g. CD4+/IL-2+, CD4+/IFNg+/TNFa+) as assessed by flow cytometry, and log 2 fold change was calculated with respect to the control condition with a non-targeting gRNA (non-targeting_sp_1). Darker shades correspond to higher cytokine expression, as indicated in the legend. Each condition was assigned a cumulative score according to all cytokine expression measurements and ranked from low to high cytokine expression. As shown in FIG. 11A, multiple DNA-targeting systems with different gRNAs increased cytokine expression in comparison to a control non-targeting gRNA, including with gRNAs targeting the genes EOMES, IL-2, TBX21, VAV1, BATF, LCP2, or CD28.

As shown in FIG. 11B, transiently expressed DNA-targeting systems for activation of IL-2 or repression of MED12 in CAR T cells led to increased and sustained killing of antigen-expressing target cells in serial stimulation assays (performed as described above in Example 5). For activation of IL-2, CAR T cells were electroporated with a transiently expressed DNA-targeting system containing dSpCas9-2xVP64 and a gRNA targeting IL-2 (IL-21, targeting SEQ ID NO:78). For repression of MED12, CAR T cells were electroporated with a transiently expressed DNA-targeting system containing dSpCas9-KRAB and a gRNA targeting MED12 (MED12_2, targeting SEQ ID NO:81). Control cells did not receive the DNA-targeting system (CAR alone) or did not express a CAR (Mock). FIG. 11B shows quantification of growth of fluorescent antigen-expressing target cells as determined using the Incucyte automated tracking system. Target cell growth was quantified based on fluorescence every 2 hours and throughout the serial stimulations. After a first stimulation, as expected, control (Mock) cells without a CAR failed to kill antigen-expressing cells, which proliferated. In contrast, CAR T cells effectively killed target antigen-expressing cells, with target cell numbers decreasing over time. After a second and third stimulation, CAR T cells not delivered with a DNA-targeting system (CAR alone) exhibited reduced killing, indicating exhaustion of the T cells. In contrast, CAR T cells delivered with a DNA-targeting system for IL-2 activation or MED12 repression retained killing activity after a second and third stimulation. Fold expansion of the CAR T cells was quantified after the second stimulation, based on counts of CAR T cells at the onset and end of the second stimulation period. As shown in FIG. 11C (left panel), CAR T cells delivered with the DNA-targeting system for IL-2 activation or the DNA-targeting system for MED12 repression expanded by approximately 2-fold during the second stimulation, as compared to control cells, which exhibited reduced numbers (mock), or failed to expand (CAR alone). CAR T cell IL-2 secretion was also quantified 24 hours after the second stimulation by MSD immunoassay. As shown in FIG. 11C (right panel), CAR T cells delivered with the DNA-targeting system for IL-2 activation or the DNA-targeting system for MED12 repression exhibited a dramatic increase in secreted IL-2 levels in comparison to both CAR alone and Mock control cells.

The results support using the DNA-targeting systems for activation or repression of target genes for driving advantageous cellular phenotypes in T cells, including increased cytokine expression and cytotoxic activity, for example in cellular therapies.

### Example 6: Modulation of T Cell Phenotypes with DNA-Targeting Systems Including Multiplexed DNA-Targeting Systems

DNA-targeting systems for individual or multiplexed gene activation or individual or multiplexed gene repression were tested to identify systems that modulate phenotypes related to T cell effector function. The DNA-targeting systems contained gRNAs or combinations thereof identified in the previous Examples.

### A. DNA-Targeting Systems for Individual or Multiplexed Gene Activation of IL-2 and VAV1 for Promoting Polyfunctional Cytokine Production

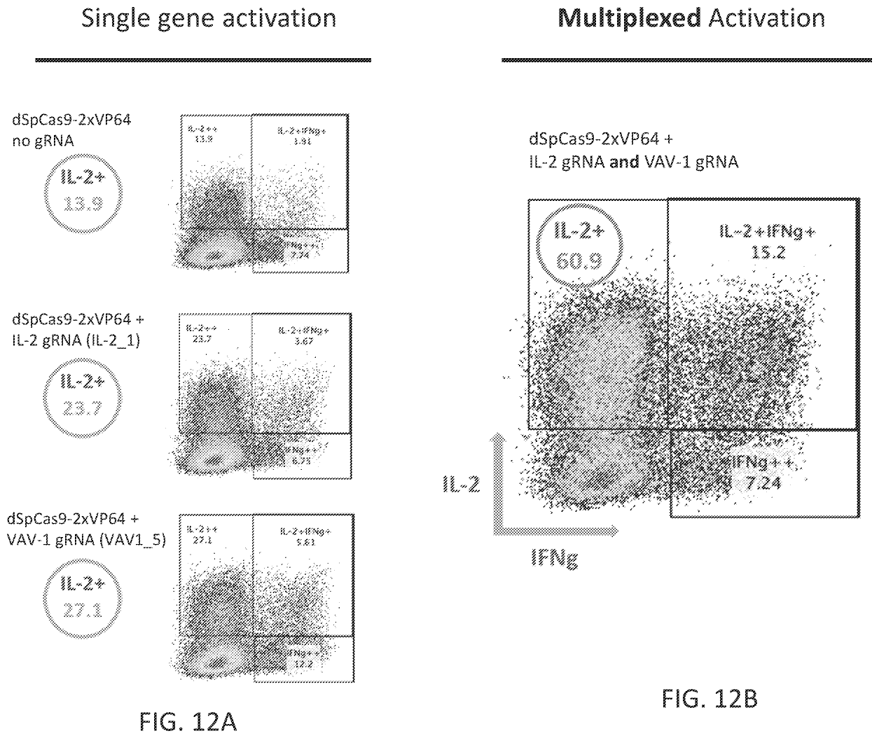

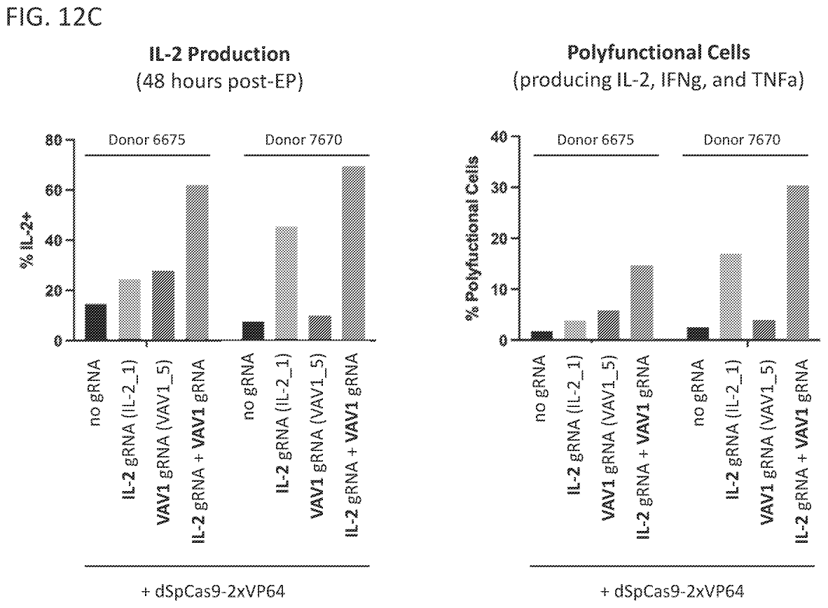

DNA-targeting systems containing gRNAs for targeting a target site in VAV1 (gRNA VAV1_5, targeting SEQ ID NO:170), IL-2 (gRNA IL-2_1, targeting SEQ ID NO:78), or both VAV1 and IL-2, and a dSpCas9-2xVP64 effector fusion protein (SEQ ID NO:76, encoding SEQ ID NO:77) were transiently expressed in Her2 CAR T cells, which were stimulated by co-culture with antigen-expressing tumor cell lines, as described in Example 5 above. About 48 hours after transient transfection, IL-2 and IFNg expression were assessed by ICS. ICS flow cytometry plots are shown in FIG. 12A for control dSpCas9-2xVP64 effector only (without targeting gRNA), and for DNA-targeting systems targeting either VAV1 or IL-2 individually. FIG. 12B shows the results of multiplexed targeting of both VAV1 and IL-2 with the gRNAs targeting VAV1 and IL-2 in combination with dSpCas9-2xVP64. The percent increase in IL-2 production or in polyfunctional cytokines IL-2, IFNg and TNF-alpha is shown in FIG. 12C. The results from this experiment show a synergistic increase in IL-2+ and IL-2+ IFNg+ cells compared to cells in which only one of the genes was targeted by the DNA-targeting systems.

### B. DNA-Targeting Systems for Individual or Multiplexed Gene Activation or Repression for Modulating CAR T Cell Phenotypes

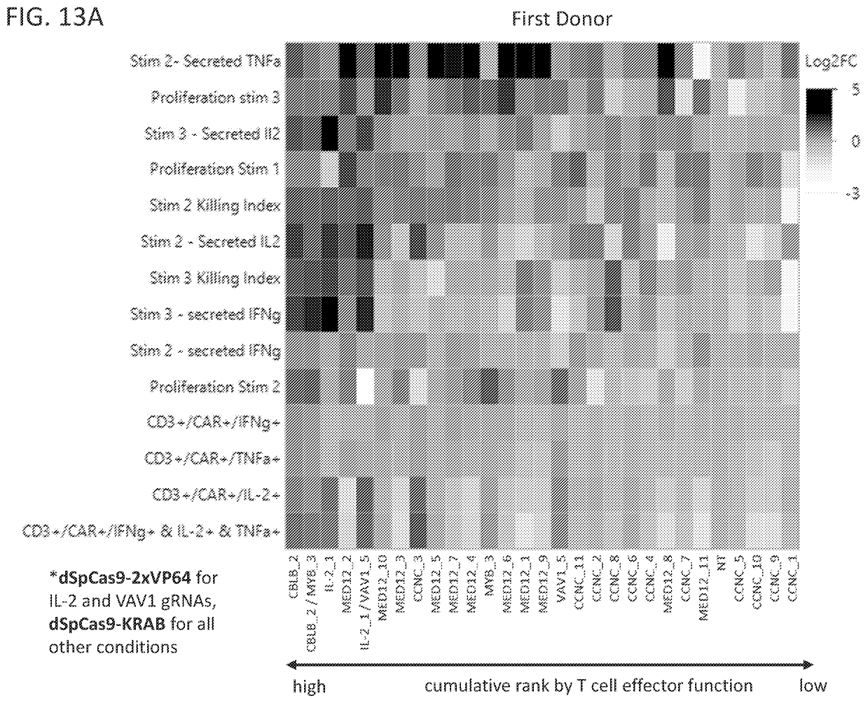

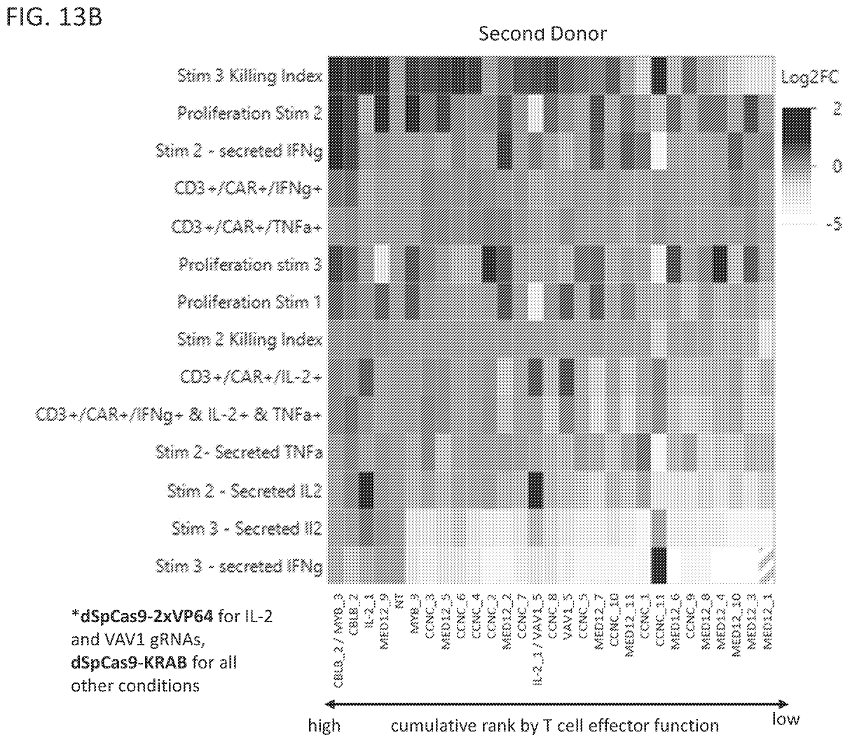

As shown in FIG. 13A and FIG. 13B, DNA-targeting systems for repression (using dSpCas9-KRAB) or activation (using dSpCas9-2xVP64) of target genes were tested to identify systems that modulate phenotypes in CAR T cells related to T cell effector function. Included were DNA-targeting systems with a single gRNA (e.g. shown in Table E2, Table E5.A, and Table E5.B), as well as multiplexed DNA-targeting systems for multiplexed activation of IL-2 and VAV-1 (containing gRNAs IL-2_1 and VAV1_5 as shown in Table E5.B), or multiplexed repression of CBLB and MYB (containing gRNAs CBLB_2 and MYB_3, as shown in Table E2). The DNA-targeting systems were electroporated for transient expression into Her2 CAR T cells stimulated by co-culture with a Her2 expressing tumor cell line. Multiple rounds of stimulation were performed, and cells electroporated with the different DNA-targeting systems were assessed based on multiple readouts of T cell effector function after the stimulations, including intracellular cytokine expression (by ICS), cytokine secretion, proliferation, and killing of target cells. The readouts of T cell effector function were assessed after a first, second, and/or third stimulation (shown as stim 1, stim 2, or stim 3 in figures). ICS was performed after a first stimulation only. Assays were performed using CAR T cells derived from a first donor (FIG. 13A) and a second donor (FIG. 13B). The results are shown as Log 2 fold change in comparison to control conditions with a non-targeting gRNA (NT). Darker shades correspond to increased measured T cell effector functions, as indicated in the legend. Each condition was assigned a cumulative score according to all measurements and ranked from low to high T cell effector function. As shown in FIG. 13A and FIG. 13B, multiple DNA-targeting systems containing different gRNAs increased T cell effector functions, including with gRNAs targeting the genes IL-2 or VAV1 for activation, or CBLB, MED12, MYB, or CCNC for repression. In addition, in this experiment, the multiplexed DNA-targeting systems outperformed many of the non-multiplexed DNA-targeting systems, resulting in increased cytokine production, proliferation, and/or killing. In addition, multiplexed repression of CBLB and MYB resulted in higer cytokine production, proliferation, and/or killing than repression of either CBLB or MYB alone in CAR T cells from the second donor.

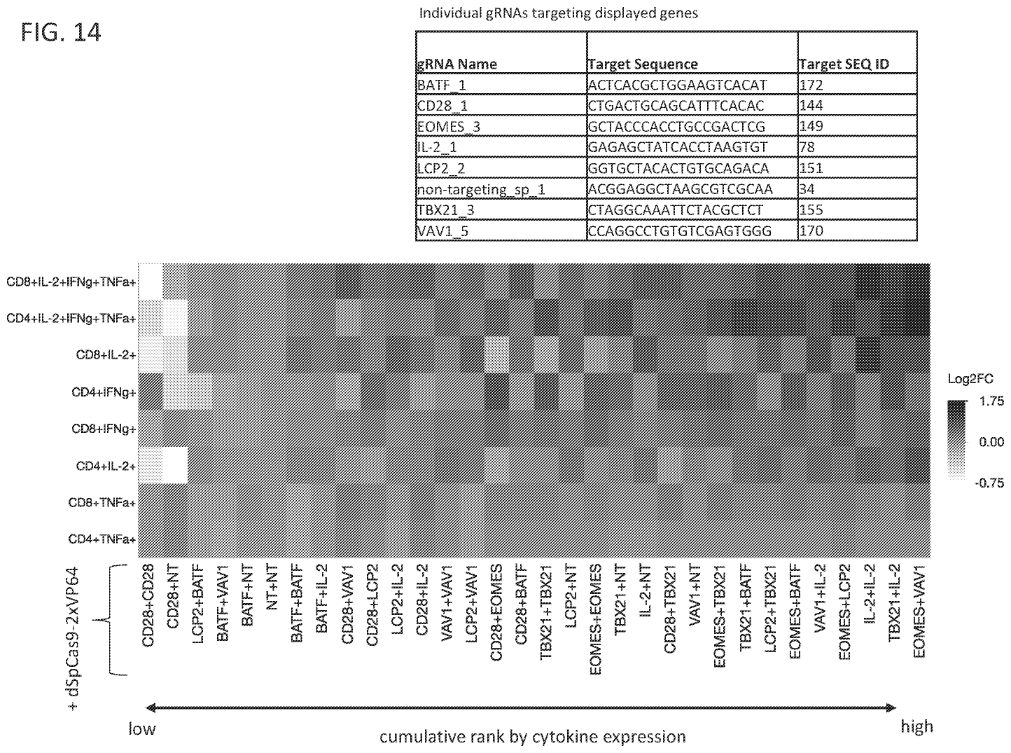

As shown in FIG. 14, additional DNA-targeting systems for multiplexed activation of combinations of individual genes using gRNAs identified in the preceding Examples and dSpCas9-2xVP64 were assessed for ability to modulate cytokine production in stimulated Her2 CAR T cells. Each DNA-targeting system included dSpCas9-2xVP64 and two gRNAs as indicated for each condition in Table E6. Where the two gRNAs for a condition are the same, the gRNA was delivered at twice the concentration. Control non-multiplexed DNA-targeting systems included one gene-targeting gRNA and one non-targeting gRNA (NT). A control non-targeting system with non-targeting gRNA only was also included (NT+NT). Each condition was assessed for cytokine expression (by ICS) for a number of cytokine expression profiles (e.g. CD4+/TNFa+). The results are shown as Log 2 fold change in comparison to control conditions with non-targeting gRNA only (NT+NT). Darker shades correspond to higher cytokine expression, as indicated in the legend. Each condition was assigned a cumulative score according to all cytokine expression measurements and ranked from low to high cytokine expression. As shown in FIG. 14, in this experiment, several of the DNA-targeting systems, including multiplexed DNA-targeting systems, increased cytokine production in comparison to the non-targeting system. Furthermore, in this experiment, DNA-targeting systems for multiplexed activation of a first gene and second gene using two different gene-targeting gRNAs led to higher cytokine production than DNA-targeting systems including only one of the gene-targeting gRNAs and optionally a second non-targeting gRNA, for several of the combinations.

Table E6 shows target genes and gRNAs for each condition shown in FIG. 14.

TABLE E6
Target genes and gRNAs corresponding to FIG. 14

| 1st target gene; | | |
|---|---|---|
| 2nd target gene | 1st gRNA (and target site SEQ ID NO:) | 2nd gRNA (and target site SEQ ID NO:) |
| BATF; BATF | BATF_1 (SEQ ID NO: 172) | BATF_1 (SEQ ID NO: 172) |
| BATF; IL-2 | BATF_1 (SEQ ID NO: 172) | IL-2_1 (SEQ ID NO: 78) |
| BATF; NT | BATF_1 (SEQ ID NO: 172) | non-targeting_sp_1 (SEQ ID NO: 34) |
| BATF; VAV1 | BATF_1 (SEQ ID NO: 172) | VAV1_5 (SEQ ID NO: 170) |
| CD28; BATF | CD28_1 (SEQ ID NO: 144) | BATF_1 (SEQ ID NO: 172) |
| CD28; CD28 | CD28_1 (SEQ ID NO: 144) | CD28_1 (SEQ ID NO: 144) |
| CD28; EOMES | CD28_1 (SEQ ID NO: 144) | EOMES_3 (SEQ ID NO: 149) |
| CD28; IL-2 | CD28_1 (SEQ ID NO: 144) | IL-2_1 (SEQ ID NO: 78) |
| CD28; LCP2 | CD28_1 (SEQ ID NO: 144) | LCP2_2 (SEQ ID NO: 151) |
| CD28; NT | CD28_1 (SEQ ID NO: 144) | non-targeting_sp_1 (SEQ ID NO: 34) |
| CD28; TBX21 | CD28_1 (SEQ ID NO: 144) | TBX21_3 (SEQ ID NO: 155) |
| CD28; VAV1 | CD28_1 (SEQ ID NO: 144) | VAV1_5 (SEQ ID NO: 170) |
| EOMES; BATF | EOMES_3 (SEQ ID NO: 149) | BATF_1 (SEQ ID NO: 172) |
| EOMES; EOMES | EOMES_3 (SEQ ID NO: 149) | EOMES_3 (SEQ ID NO: 149) |
| EOMES; LCP2 | EOMES_3 (SEQ ID NO: 149) | LCP2_2 (SEQ ID NO: 151) |
| EOMES; TBX21 | EOMES_3 (SEQ ID NO: 149) | TBX21_3 (SEQ ID NO: 155) |
| EOMES; VAV1 | EOMES_3 (SEQ ID NO: 149) | VAV1_5 (SEQ ID NO: 170) |
| IL-2; IL-2 | IL-2_1 (SEQ ID NO: 78) | IL-2_1 (SEQ ID NO: 78) |
| IL-2; NT | IL-2_1 (SEQ ID NO: 78) | non-targeting_sp_1 (SEQ ID NO: 34) |
| LCP2; BATF | LCP2_2 (SEQ ID NO: 151) | BATF_1 (SEQ ID NO: 172) |
| LCP2; IL-2 | LCP2_2 (SEQ ID NO: 151) | IL-2_1 (SEQ ID NO: 78) |
| LCP2; NT | LCP2_2 (SEQ ID NO: 151) | non-targeting_sp_1 (SEQ ID NO: 34) |
| LCP2; TBX21 | LCP2_2 (SEQ ID NO: 151) | TBX21_3 (SEQ ID NO: 155) |
| LCP2; VAV1 | LCP2_2 (SEQ ID NO: 151) | VAV1_5 (SEQ ID NO: 170) |
| NT; NT | non-targeting_sp_1 (SEQ ID NO: 34) | non-targeting_sp_1 (SEQ ID NO: 34) |
| TBX21; BATF | TBX21_3 (SEQ ID NO: 155) | BATF_1 (SEQ ID NO: 172) |
| TBX21; IL-2 | TBX21_3 (SEQ ID NO: 155) | IL-2_1 (SEQ ID NO: 78) |
| TBX21; NT | TBX21_3 (SEQ ID NO: 155) | non-targeting_sp_1 (SEQ ID NO: 34) |
| TBX21; TBX21 | TBX21_3 (SEQ ID NO: 155) | TBX21_3 (SEQ ID NO: 155) |
| VAV1; IL-2 | VAV1_5 (SEQ ID NO: 170) | IL-2_1 (SEQ ID NO: 78) |
| VAV1; NT | VAV1_5 (SEQ ID NO: 170) | non-targeting_sp_1 (SEQ ID NO: 34) |
| VAV1; VAV1 | VAV1_5 (SEQ ID NO: 170) | VAV1_5 (SEQ ID NO: 170) |

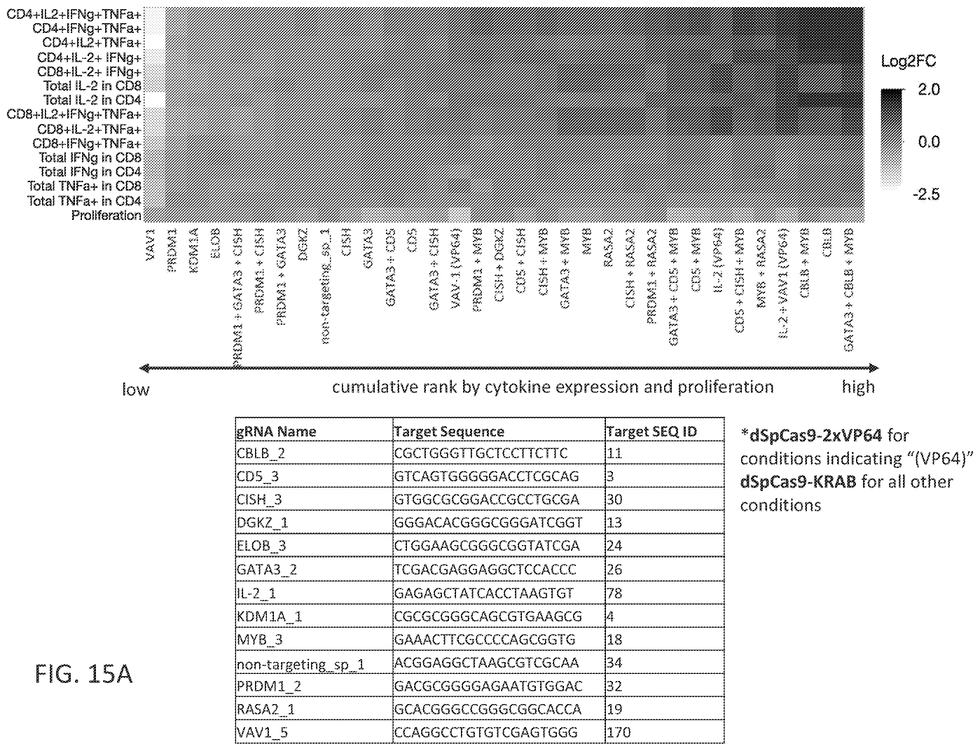

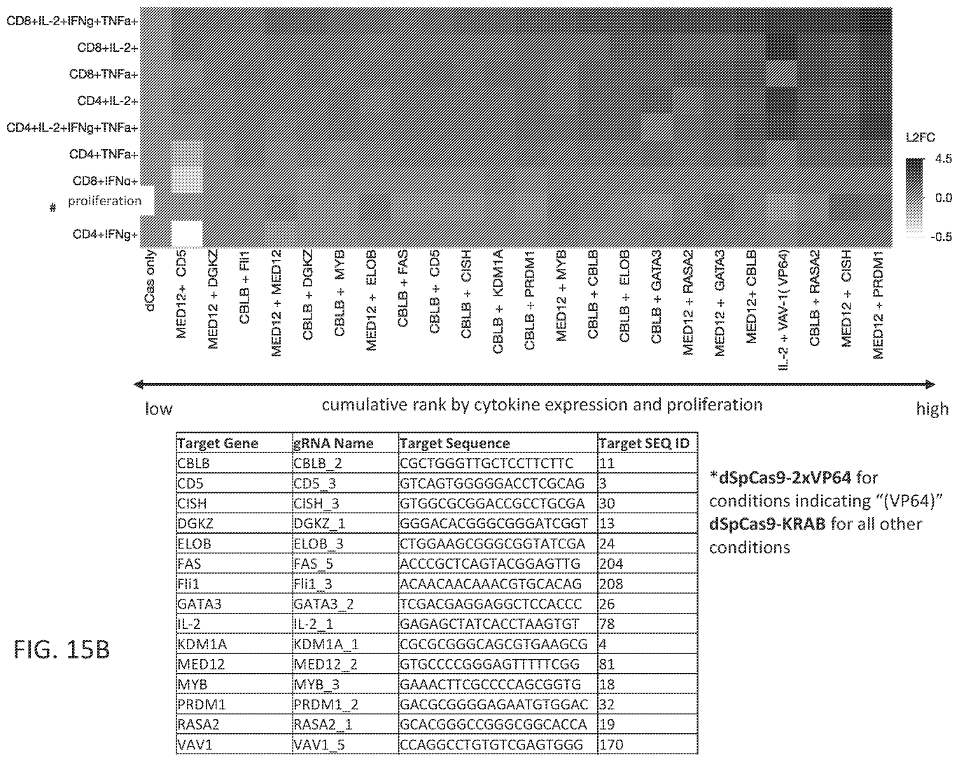

As shown in FIG. 15A and FIG. 15B, additional DNA-targeting systems for multiplexed activation or multiplexed repression of combinations of individual genes using gRNAs identified in the preceding Examples and dSpCas9-2xVP64 (for activation) or dSpCas9-KRAB (for repression), were assessed for ability to modulate phenotypes in stimulated Her2 CAR T cells in comparison to control conditions with a single gRNA, a non-targeting gRNA, or no gRNA (dCas only). Target genes and gRNAs for each condition in FIG. 15A are shown in Table E7T Target genes and gRNAs for each condition in FIG. 15B are shown in Table E8. Each condition was assessed for cytokine expression (by ICS) and proliferation. Results are shown as Log 2 fold change in comparison to control conditions (non-targeting gRNA in FIG. 15A; dCas only in FIG. 15B). Darker shades correspond to increased proliferation and cytokine expression, as indicated in the legend. Each condition was assigned a cumulative score according to all cytokine and proliferation measurements and ranked from low to high cytokine expression and proliferation. As shown in FIG. 15A and FIG. 15B, in this experiment, several of the DNA-targeting systems, including multiplexed DNA-targeting systems, increased CAR T cell proliferation and/or cytokine production in comparison to control conditions. Furthermore, DNA-targeting systems for multiplexed activation or multiplexed repression of at least a first gene and second gene using different gene-targeting gRNAs led to higher proliferation and/or cytokine production than DNA-targeting systems including only one of the gene-targeting gRNAs, for several of the combinations.

TABLE E7
Target genes and gRNAs corresponding to FIG. 15A

| Targeted Gene(s) | Effector | gRNA name(s) (and target site SEQ ID NO) |
|---|---|---|
| CBLB | KRAB | CBLB_2 (SEQ ID NO: 11) |
| CBLB; MYB | KRAB | CBLB_2 (SEQ ID NO: 11); MYB_3 (SEQ ID NO: 18) |
| CD5 | KRAB | CD5_3 (SEQ ID NO: 3) |
| CD5; CISH | KRAB | CD5_3 (SEQ ID NO: 3); CISH_3 (SEQ ID NO: 30) |
| CD5; CISH; | KRAB | CD5_3 (SEQ ID NO: 3); CISH_3 (SEQ ID NO: 30); MYB_3 (SEQ ID |
| MYB | | NO: 18) |
| CD5; MYB | KRAB | CD5_3 (SEQ ID NO: 3); MYB_3 (SEQ ID NO: 18) |
| CISH | KRAB | CISH_3 (SEQ ID NO: 30) |
| CISH; DGKZ | KRAB | CISH_3 (SEQ ID NO: 30); DGKZ_1 (SEQ ID NO: 13) |
| CISH; MYB | KRAB | CISH_3 (SEQ ID NO: 30); MYB_3 (SEQ ID NO: 18) |
| CISH; RASA2 | KRAB | CISH_3 (SEQ ID NO: 30); RASA2_1 (SEQ ID NO: 19) |
| DGKZ | KRAB | DGKZ_1 (SEQ ID NO: 13) |
| ELOB | KRAB | ELOB_3 (SEQ ID NO: 24) |
| GATA3 | KRAB | GATA3_2 (SEQ ID NO: 26) |
| GATA3; CBLB; | KRAB | GATA3_2 (SEQ ID NO: 26); CBLB_2 (SEQ ID NO: 11); MYB_3 (SEQ |
| MYB | | ID NO: 18) |
| GATA3; CD5 | KRAB | GATA3_2 (SEQ ID NO: 26); CD5_3 (SEQ ID NO: 3) |
| GATA3; CD5; | KRAB | GATA3_2 (SEQ ID NO: 26); CD5_3 (SEQ ID NO: 3); MYB_3 (SEQ |
| MYB | | ID NO: 18) |
| GATA3; CISH | KRAB | GATA3_2 (SEQ ID NO: 26); CISH_3 (SEQ ID NO: 30) |
| GATA3; MYB | KRAB | GATA3_2 (SEQ ID NO: 26); MYB_3 (SEQ ID NO: 18) |
| IL-2 | VP64 | IL-2_1 (SEQ ID NO: 78) |
| IL-2; VAV1 | VP64 | IL-2_1 (SEQ ID NO: 78); VAV1_5 (SEQ ID NO: 170) |
| KDM1A | KRAB | KDM1A_1 (SEQ ID NO: 4) |
| MYB | KRAB | MYB_3 (SEQ ID NO: 18) |
| MYB; RASA2 | KRAB | MYB_3 (SEQ ID NO: 18); RASA2_1 (SEQ ID NO: 19) |
| non-targeting | KRAB | non-targeting_sp_1 (SEQ ID NO: 34) |
| gRNA | | |
| PRDM1 | KRAB | PRDM1_2 (SEQ ID NO: 32) |
| PRDM1; CISH | KRAB | PRDM1_2 (SEQ ID NO: 32); CISH_3 (SEQ ID NO: 30) |
| PRDM1; | KRAB | PRDM1_2 (SEQ ID NO: 32); GATA3_2 (SEQ ID NO: 26) |
| GATA3 | | |
| PRDM1; | KRAB | PRDM1_2 (SEQ ID NO: 32); GATA3_2 (SEQ ID NO: 26); CISH_3 |
| GATA3; CISH | | (SEQ ID NO: 30) |
| PRDM1; MYB | KRAB | PRDM1_2 (SEQ ID NO: 32); MYB_3 (SEQ ID NO: 18) |
| PRDM1; | KRAB | PRDM1_2 (SEQ ID NO: 32); RASA2_1 (SEQ ID NO: 19) |
| RASA2 | | |
| RASA2 | KRAB | RASA2_1 (SEQ ID NO: 19) |
| VAV1 | KRAB | VAV1_5 (SEQ ID NO: 170) |
| VAV1 | VP64 | VAV1_5 (SEQ ID NO: 170) |

TABLE E8
Target genes and gRNAs corresponding to FIG. 15B

| 1st target gene; | | 1st gRNA | 2nd gRNA |
|---|---|---|---|
| 2nd target gene | Effector | (and target site SEQ ID NO:) | (and target site SEQ ID NO:) |
| CBLB; CBLB | KRAB | CBLB_2 (SEQ ID NO: 11) | CBLB_2 (SEQ ID NO: 11) |
| CBLB; CD5 | KRAB | CBLB_2 (SEQ ID NO: 11) | CD5_3 (SEQ ID NO: 3) |
| CBLB; CISH | KRAB | CBLB_2 (SEQ ID NO: 11) | CISH_3 (SEQ ID NO: 30) |
| CBLB; DGKZ | KRAB | CBLB_2 (SEQ ID NO: 11) | DGKZ_1 (SEQ ID NO: 13) |
| CBLB; ELOB | KRAB | CBLB_2 (SEQ ID NO: 11) | ELOB_3 (SEQ ID NO: 24) |
| CBLB; FAS | KRAB | CBLB_2 (SEQ ID NO: 11) | FAS_5 (SEQ ID NO: 204) |
| CBLB; Fli1 | KRAB | CBLB_2 (SEQ ID NO: 11) | Fli1_3 (SEQ ID NO: 208) |
| CBLB; GATA3 | KRAB | CBLB_2 (SEQ ID NO: 11) | GATA3_2 (SEQ ID NO: 26) |
| CBLB; KDM1A | KRAB | CBLB_2 (SEQ ID NO: 11) | KDM1A_1 (SEQ ID NO: 4) |
| CBLB; MYB | KRAB | CBLB_2 (SEQ ID NO: 11) | MYB_3 (SEQ ID NO: 18) |
| CBLB; PRDM1 | KRAB | CBLB_2 (SEQ ID NO: 11) | PRDM1_2 (SEQ ID NO: 32) |
| CBLB; RASA2 | KRAB | CBLB_2 (SEQ ID NO: 11) | RASA2_1 (SEQ ID NO: 19) |
| IL-2; VAV1 (VP64) | VP64 | IL-2_1 (SEQ ID NO: 78) | VAV1_5 (SEQ ID NO: 170) |
| MED12; CBLB | KRAB | MED12_2 (SEQ ID NO: 81) | CBLB_2 (SEQ ID NO: 11) |
| MED12; CD5 | KRAB | MED12_2 (SEQ ID NO: 81) | CD5_3 (SEQ ID NO: 3) |
| MED12; CISH | KRAB | MED12_2 (SEQ ID NO: 81) | CISH_3 (SEQ ID NO: 30) |
| MED12; DGKZ | KRAB | MED12_2 (SEQ ID NO: 81) | DGKZ_1 (SEQ ID NO: 13) |
| MED12; ELOB | KRAB | MED12_2 (SEQ ID NO: 81) | ELOB_3 (SEQ ID NO: 24) |
| MED12; GATA3 | KRAB | MED12_2 (SEQ ID NO: 81) | GATA3_2 (SEQ ID NO: 26) |
| MED12; MED12 | KRAB | MED12_2 (SEQ ID NO: 81) | MED12_2 (SEQ ID NO: 81) |
| MED12; MYB | KRAB | MED12_2 (SEQ ID NO: 81) | MYB_3 (SEQ ID NO: 18) |
| MED12; PRDM1 | KRAB | MED12_2 (SEQ ID NO: 81) | PRDM1_2 (SEQ ID NO: 32) |
| MED12; RASA2 | KRAB | MED12_2 (SEQ ID NO: 81) | RASA2_1 (SEQ ID NO: 19) |

### C. DNA-Targeting Systems for Multiplexed Gene Activation of IL-2 and VAV1, or Multiplexed Repression of Combinations of CBLB, CCNC, MED12, and MYB

DNA-targeting systems for individual or multiplexed gene activation (with dSpCas9-2xVP64) of IL-2, VAV1, or a combination of IL-2 and VAV-1, or individual or multiplexed gene repression (with dSpCas9-KRAB) of CBLB, CCNC, MED12, MYB, or a combination thereof, were further characterized for ability to modulate phenotypes in CAR T cells related to T cell effector function. The DNA-targeting systems containing gene-targeting gRNAs and dSpCas9 effector fusion proteins for targeted activation or repression were electroporated for transient expression in Her2 CAR T cells that were stimulated with antigen expressing target cells, generally as described above. The CAR T cells were assessed based on proliferation (fold expansion), cytokine expression (both ICS and cytokine secretion) and ability to kill antigen-expressing target cells in co-culture. Negative control conditions included CAR T cells delivered with a dCas-effector and no gRNA, CAR T cells not delivered with a DNA-targeting system (CAR only), and cells not expressing a CAR (mock). Target genes and corresponding gRNAs for these experiments are shown in Table E9.

TABLE E9
Target genes and gRNAs corresponding to FIGS. 16-19.

| Target | gRNA | | Target | Repression/ |
|---|---|---|---|---|
| Gene | Name | Target Sequence | SEQ ID | Activation |
| IL-2 | IL-2_1 | GAGAGCTATCACCTAAGTGT | 78 | Activation |
| VAV1 | VAV1_5 | CCAGGCCTGTGTCGAGTGGG | 170 | Activation |
| CBLB | CBLB_2 | CGCTGGGTTGCTCCTTCTTC | 11 | Repression |
| CCNC | CCNC_3 | CGACGGCGAAAGGAAGAGGA | 104 | Repression |
| MED12 | MED12_2 | GTGCCCCGGGAGTTTTTCGG | 81 | Repression |
| MYB | MYB_3 | GAAACTTCGCCCCAGCGGTG | 18 | Repression |

As shown in FIG. 16, in this experiment, DNA-targeting systems, including multiplexed DNA-targeting systems, led to increased proliferation (fold expansion) of CAR T cells in comparison to negative control conditions in a first donor and a second donor. Proliferation data in FIG. 16 was measured after a first stimulation with target cells (stim 1). Multiplexed repression of CBLB and MED12 dramatically increased proliferation by 5-10 fold in comparison to controls.

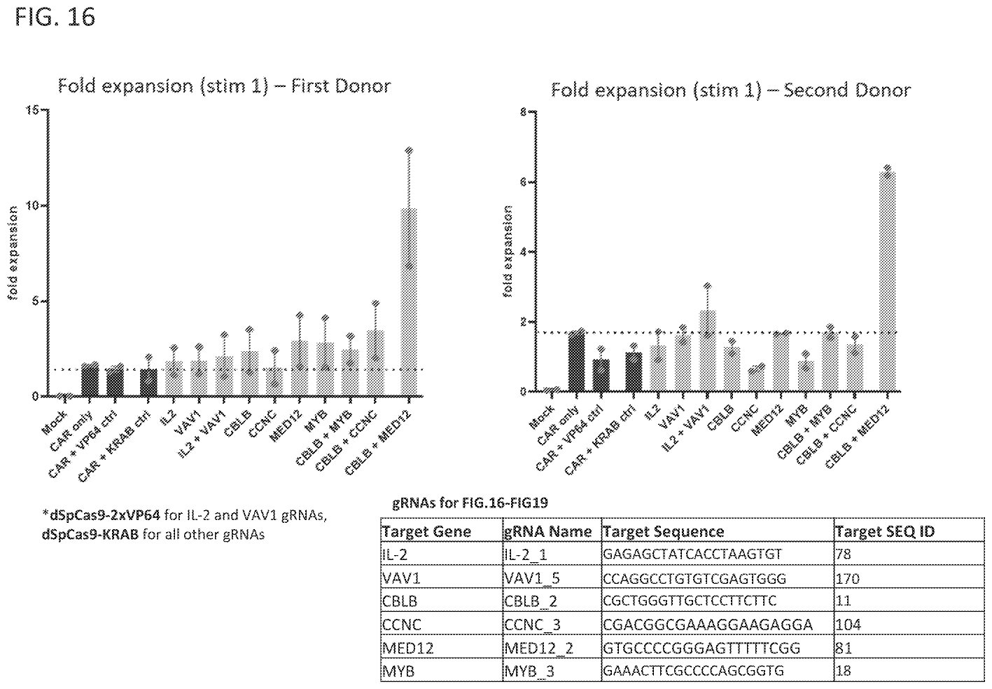

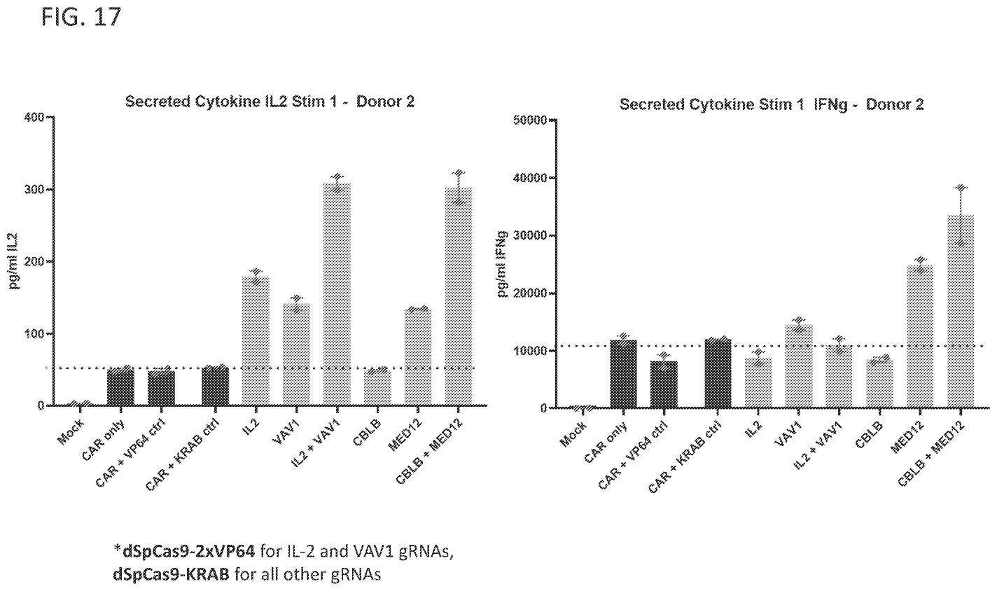

As shown in FIG. 17, the DNA-targeting systems for individual or multiplexed gene modulation were further characterized based on ability to modulate secreted cytokines IL-2 and IFNg in CAR T cells. Cytokine secretion was measured by MSD immunoassays, as described above in Example 5. Cyokine data in FIG. 17 was measured after a first stimulation with target cells (stim 1). In this experiment, DNA-targeting systems, including multiplexed DNA-targeting systems, increased cytokine secretion in comparison to negative control conditions. Multiplexed activation of IL-2 and VAV1 led to higher secretion of IL-2 than activation of either gene alone. Multiplexed repression of CBLB and MED12 led to higher secretion of both IL-2 and IFNg than repression of either gene alone.

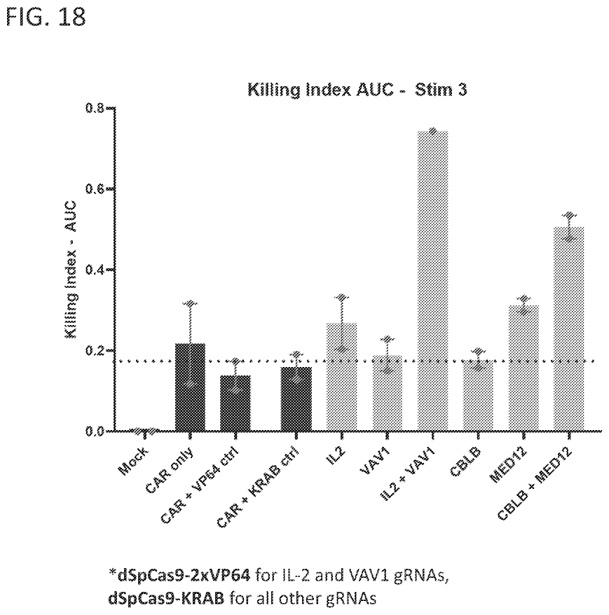

As shown in FIG. 18, the DNA-targeting systems for individual or multiplexed gene modulation were further characterized based on ability of electroporated CAR T cells to kill antigen expressing target cells after a third round of stimulation (a model for T cell exhaustion), generally as described above in Example 5. A killing index based on the growth curve of antigen-expressing target cells (area under the curve; AUC) was determined for each condition, as shown in FIG. 18. In this experiment, DNA-targeting systems, including multiplexed DNA-targeting systems, increased killing activity in comparison to negative control conditions. Multiplexed activation of IL-2 and VAV1 led to greater killing activity than activation of either gene alone. Multiplexed repression of CBLB and MED12 led to greater killing activity than repression of either gene alone. These results are consistent with a finding that the DNA-targeting systems may improve persistence of antigen-targeted T cells following exposure and activation by target cells.

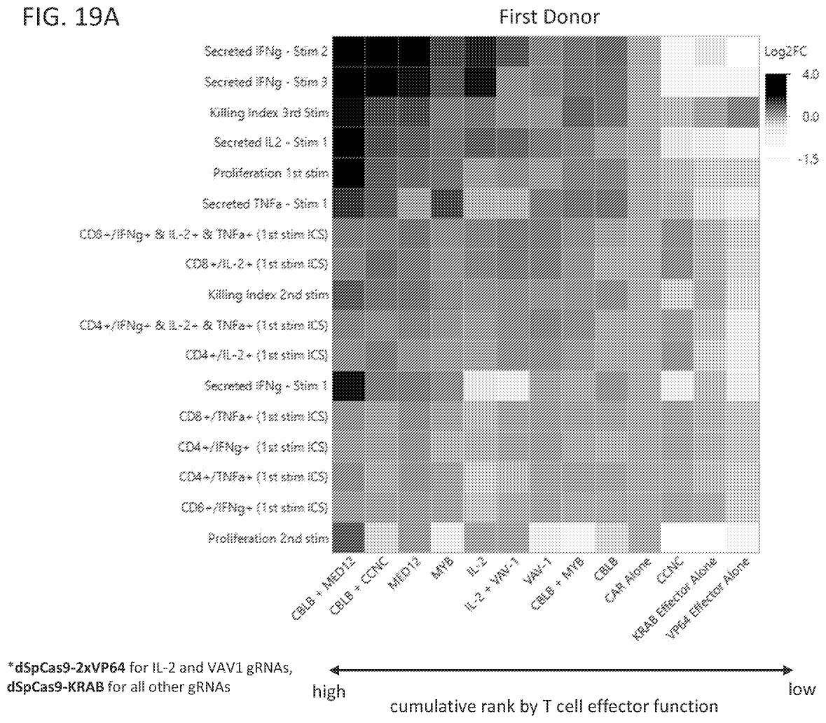

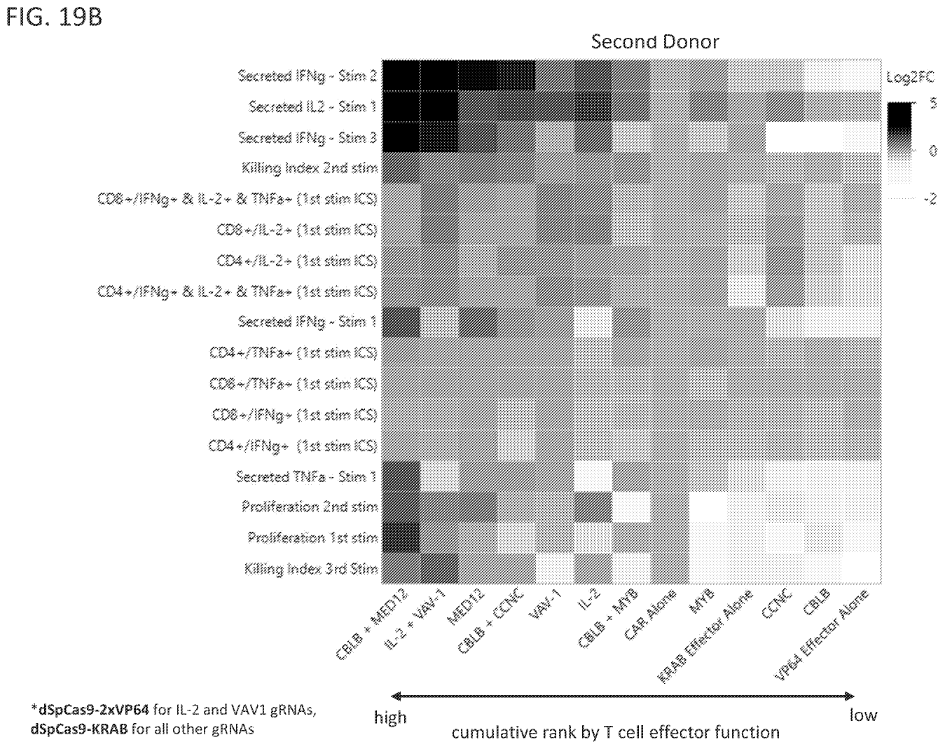

As shown in FIGS. 19A-19B, the DNA-targeting systems for individual or multiplexed gene modulation were further characterized based on multiple phenotypic readouts of T cell effector function in electroporated CAR T cells derived from a first donor (FIG. 19A) or second donor (FIG. 19B). Cells were assessed for intracellular cytokine expression (by ICS), secreted cytokine expression, proliferation, and killing, after a first, second, and/or third stimulation, as indicated in the figures (shown as stim 1, stim 2, stim 3). Results are shown as Log 2 fold change in comparison to control conditions (CAR alone). Darker shades correspond to increased measured T cell effector functions, as indicated in the legend. Each condition was assigned a cumulative score according to all measurements and ranked from low to high T cell effector function. In this experiment, DNA-targeting systems, including multiplexed DNA-targeting systems, increased T cell effector function in comparison to control conditions. Multiplexed repression of CBLB and MED12 led to increased T cell effector function in comparison to repression of either gene alone in both donors, and multiplexed repression of CBLB and MYB led to increased T cell effector function in comparison repression of either gene alone in the second donor. Multiplexed activation of IL-2 and VAV1 led to increased T cell effector function in comparison to repression of either gene alone in the second donor.

These results demonstrate that simultaneous modifications create synergy and expand options for driving cell phenotype without the potential negative impact of DNA breaks.

D. Expansion, Killing, and Cytokine Expression in CAR T Cells Delivered with DNA-Target Systems for Repression of One or More Genes or Activation of One or More Genes

CAR T cells delivered with DNA-targeting systems for transcriptional repression of one or more genes, or DNA-targeting systems for transcriptional activation of one or more genes, were assessed for cell expansion, target cell killing, and cytokine expression.

T cells from two different donors were thawed, activated, transduced with a Her2 CAR, electroporated with DNA-targeting systems, and serially stimulated by co-culture with Her2 positive NCI-H1975 cells, generally as described above. DNA-targeting systems for repression included dSpCas9-KRAB and one or more gene-targeting gRNAs shown in Table E10. DNA-targeting systems for activation included dSpCas9-2xVP64 and one or more gRNAs shown in Table E10.

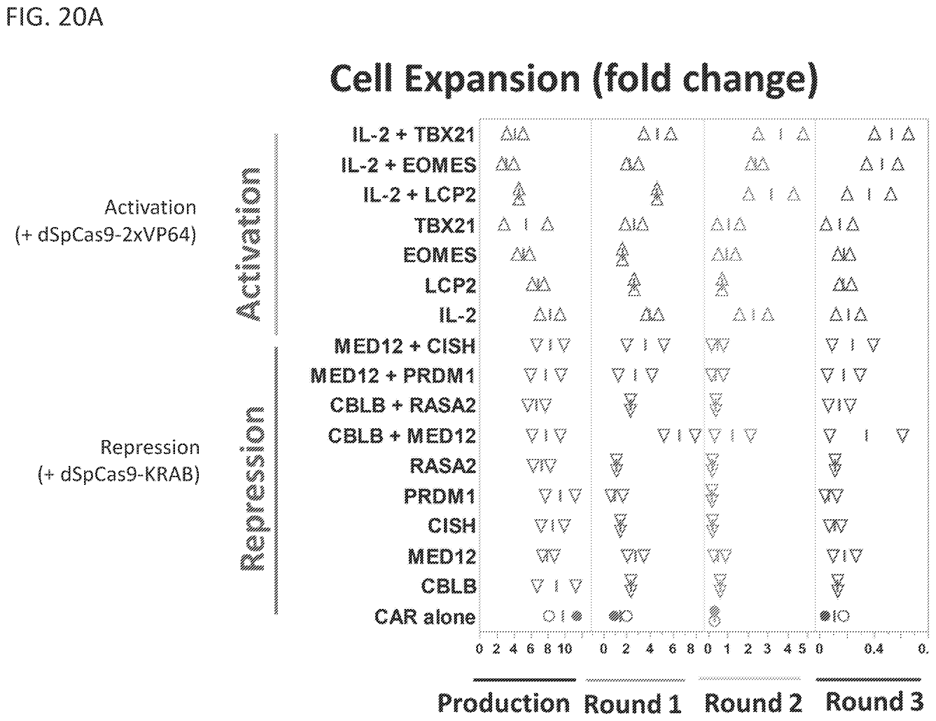

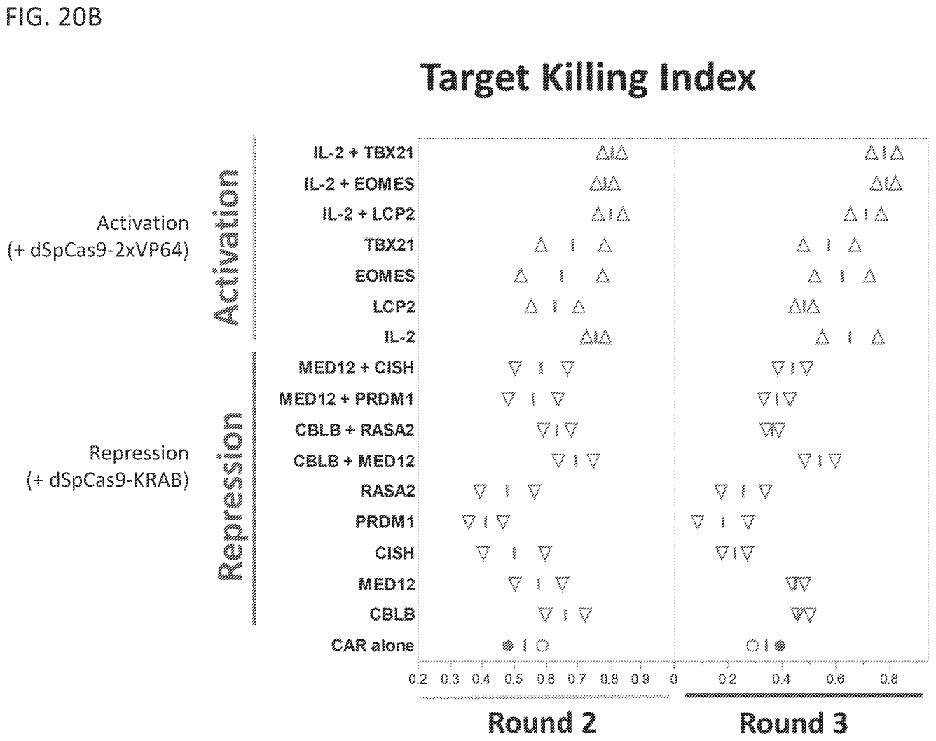

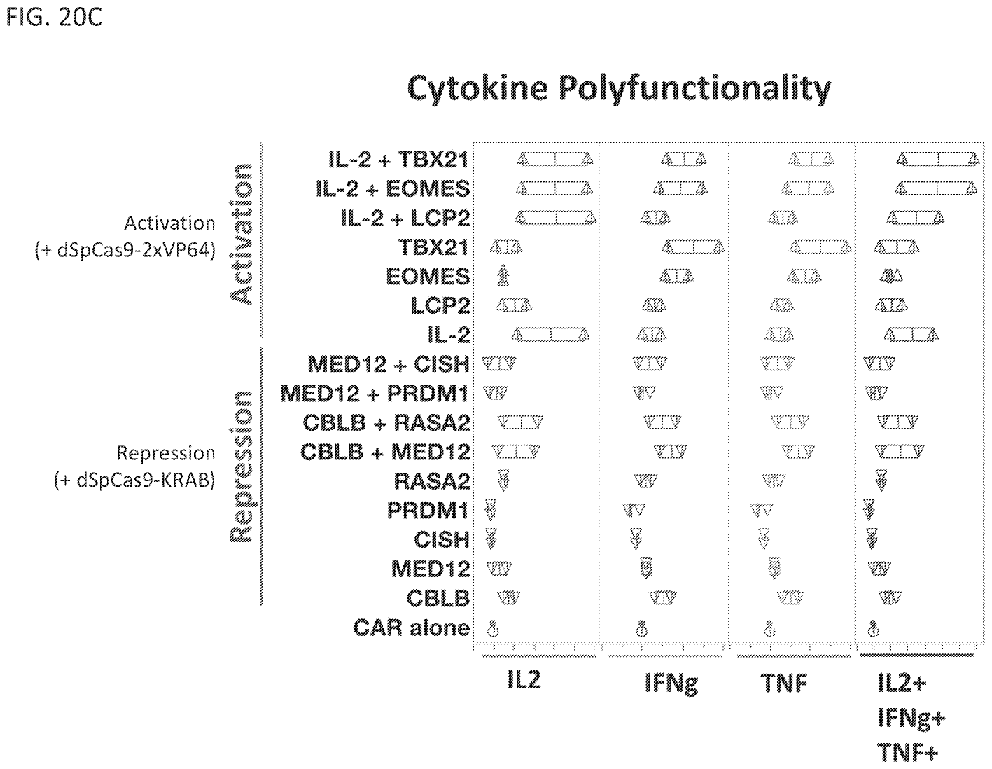

TABLE E10
Target genes and gRNAs corresponding to FIGS. 20A-C

| Target | gRNA | | Target | Repression/ |
|---|---|---|---|---|
| Gene | Name | Target Sequence | SEQ ID | Activation |
| IL-2 | IL-2_1 | GAGAGCTATCACCTAAGTG | 78 | Activation |
| | | T | | |
| TBX21 | TBX21_3 | CTAGGCAAATTCTACGCTC | 155 | Activation |
| | | T | | |
| EOMES | EOMES_ | GCTACCCACCTGCCGACTC | 149 | Activation |
| | 3 | G | | |
| LCP2 | LCP2_2 | GGTGCTACACTGTGCAGAC | 151 | Activation |
| | | A | | |
| MED12 | MED12_ | GTGCCCCGGGAGTTTTTCG | 81 | Repression |
| | 2 | G | | |
| CBLB | CBLB_2 | CGCTGGGTTGCTCCTTCTTC | 11 | Repression |
| CISH | CISH_1 | TGTCCTGCGCCCGCGCGCC | 28 | Repression |
| | | C | | |
| PRDM1 | PRDM1_ | TTGCCTCTCCGCAACACTG | 33 | Repression |
| | 3 | G | | |
| RASA2 | RASA2_ | GCACGGGCCGGGCGGCAC | 19 | Repression |
| | 1 | CA | | |

The CAR T cells were assessed for expansion (fold change in CAR T cell numbers; i.e. proliferation) during 4 different time periods, as shown in FIG. 20A. The first time period (shown as Production in FIG. 20A) was following transduction of the CAR and electroporation of the DNA-targeting system, and prior to stimulation with Her2 antigen-expressing cells. The next three time periods were during a first, second, and third stimulation with Her2 antigen-expressing cells (shown as Round 1 Round 2 and Round 3 in FIG. 20), with cell numbers being compared between the onset and the end of each stimulation. As shown in FIG. 20A, CAR T cells delivered with DNA-targeting systems for transcriptional repression or transcriptional activation exhibited increased expansion in comparison to control cells (CAR alone; not delivered with a DNA-targeting system) after one or multiple rounds of stimulation with the Her2 antigen-expressing cells. For each condition, expansion results are plotted for each of the two donors (triangles), with average value indicated by vertical line.

The CAR T cells were assessed for ability to kill Her2 antigen-expressing target cells after the second and third rounds of stimulation. A target killing index for each experimental condition with a DNA-targeting system was calculated based on the area under the growth curve (AUC) of antigen-expressing target cells in the experimental condition and mock control cells (no CAR), according to the formula: (Mock AUC−Experimental condition AUC)/Mock AUC. FIG. 20B shows killing index for CAR T cells delivered with DNA-targeting systems for activation or repression of genes with the indicated gRNAs. For each experimental condition, results are plotted for each of the two donors (triangles), with average value indicated by vertical line. As shown in FIG. 20B, CAR T cells delivered with DNA-targeting systems for transcriptional repression or transcriptional activation exhibited increased killing in comparison to control cells (CAR alone) during the second and third round of stimulation.

The CAR T cells were assessed for cytokine expression by ICS and flow cytometry at Day 9 post-electroporation with the DNA-targeting systems, and after exposure to antigen-expressing target cells. The percentage of CAR T cells that were IL-2+, IFNg+, TNF-alpha+, or IL-2+/IFNg+/TNF-alpha+(polyfunctional) was assessed. As shown in FIG. 20C, CAR T cells delivered with DNA-targeting systems for transcriptional repression or transcriptional activation exhibited increased cytokine expression in comparison to control cells (CAR alone). For each experimental condition, results are plotted for each of the two donors (triangles), with average value indicated by vertical line.

The results support using the DNA-targeting systems for activation or repression of target genes for driving advantageous cellular phenotypes in T cells, including increased proliferation, cytokine expression, cytotoxic activity, for example in cellular therapies.

### Example 7: Enhanced TGFBR2 and T Cell Effector Function with an Alternative Fusion Protein for Targeted Transcriptional Repression

DNA-targeting systems with TGFBR2-targeting gRNAs and dSpCas9 fusion proteins with different transcriptional repressor effector domains were tested for ability to repress TGFBR2 and modulate T cell effector function.

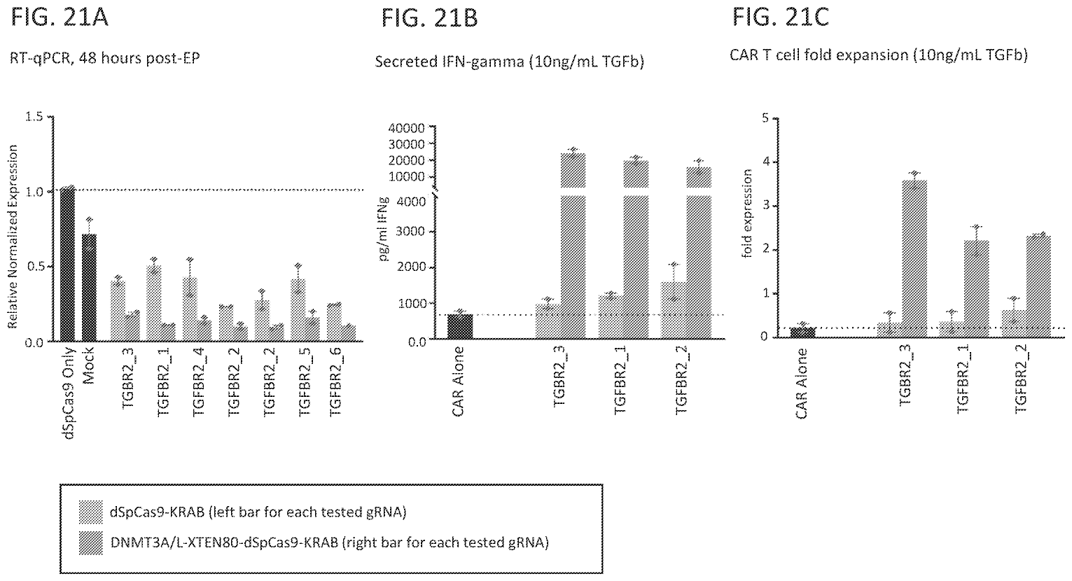

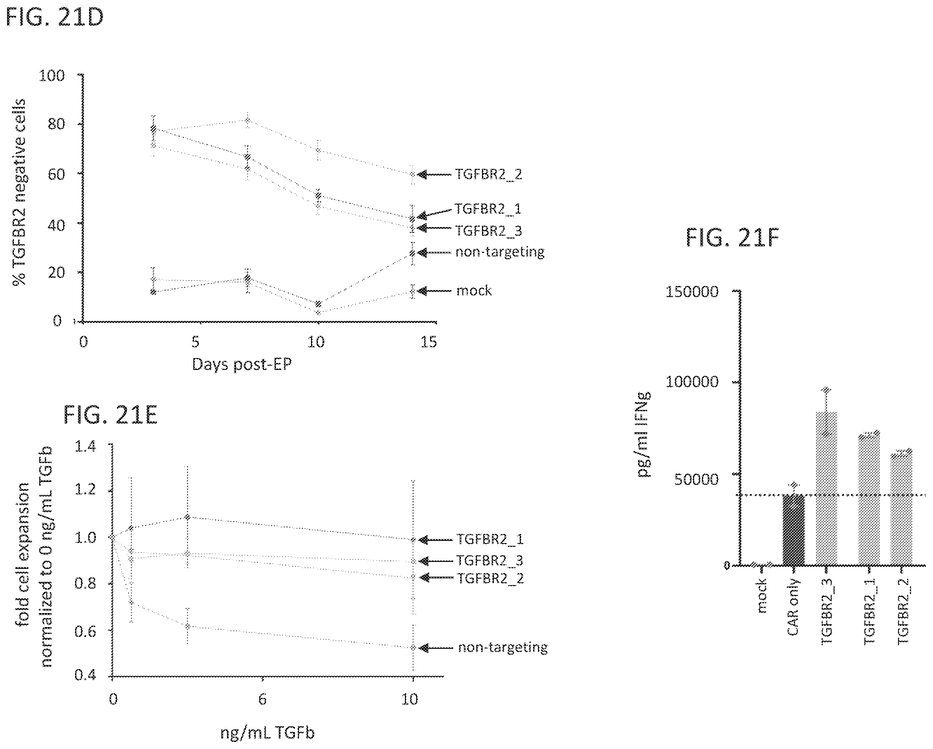

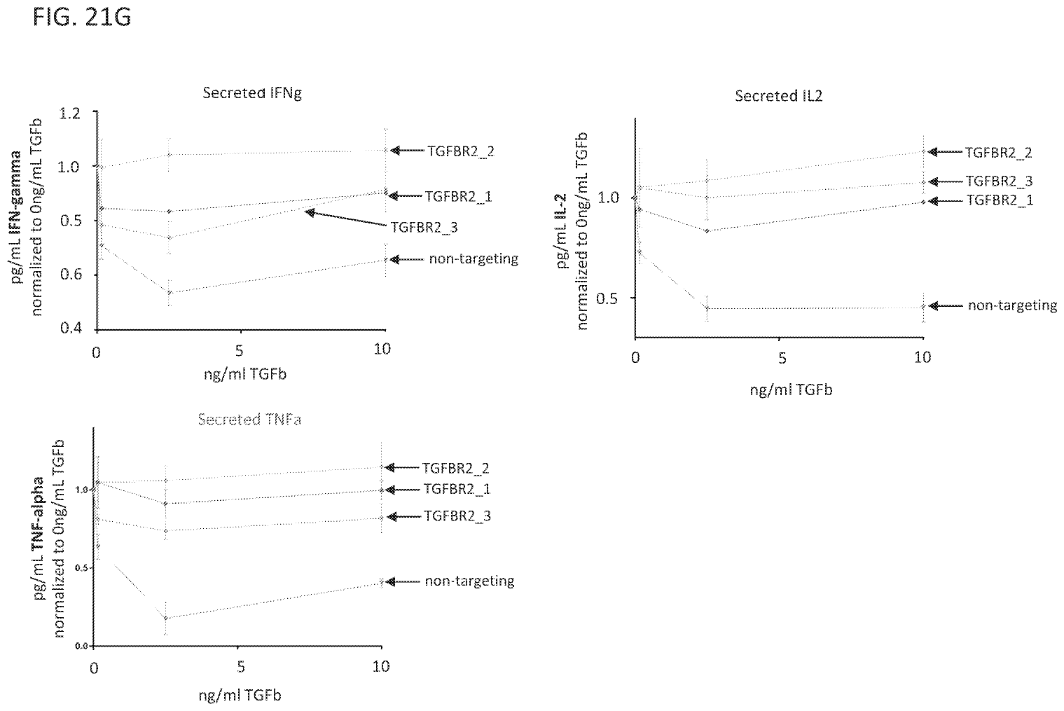

CD4 and CD8 T cells underwent CD3/CD28 activation and were transduced with Her2 CAR 24 hours later. 4 days post-transduction, CAR T cells were electroporated with DNA-targeting systems containing a TGFBR2-targeting gRNA (as shown in Table E5.A) and either a dSpCas9-KRAB fusion protein (mRNA encoding SEQ ID NO:332) or a DNMT3A/L-XTEN80-dSpCas9-KRAB fusion protein (mRNA encoding SEQ ID NO:337). Control cells included CAR T cells delivered with dSpCas9 alone (dSpCas9 Only), mock cells not transduced with a CAR (Mock), and CAR T cells not delivered with a DNA-targeting system (CAR alone). 48 hours post-electroporation, TGFBR2 expression was assessed by RT-qPCR, normalized to dSpCas9 Only controls. As shown in FIG. 21A, each of the tested TGFBR2-targeting DNA-targeting systems repressed TGFBR2. DNMT3A/L-XTEN80-dSpCas9-KRAB enhanced repression of TGFBR2 in comparison to dSpCas9-KRAB for all 7 of the tested TGFBR2-targeting gRNAs.

The CAR T cells delivered with the transiently expressed TGFBR2-targeting DNA-targeting systems with 3 of the TGFBR2-targeting gRNAs (TGFBR2_1, targeting SEQ ID NO:300; TGFBR2_2, targeting SEQ ID NO:301; and TGBR2_3, targeting SEQ ID NO:302) were serially stimulated with Her2-positive NCI H1975 tumor cells at a ratio of 1:5 CAR T:tumor cells, with serial stimulations occurring 4 days apart, in the presence of 10 ng/mL TGFb. 24 hours after the second stimulation, secreted IFN-gamma was measured by MSD immunoassay (as described above), and compared to CAR alone control cells. As shown in FIG. 21B, secreted IFNg was increased in CAR T cells delivered with each of the DNA-targetings systems, including for each of the three tested TGFBR2-targeting gRNAs and with both dSpCas9-KRAB and DNMT3A/L-XTEN80-dSpCas9-KRAB fusion proteins. In addition, DNA-targeting systems with the DNMT3A/L-XTEN80-dSpCas9-KRAB fusion protein dramatically enhanced secretion of IFNg by about 10-fold or more, in comparison to DNA-targeting systems with the dSpCas9-KRAB fusion protein. CAR T cell proliferation was measured after the second stimulation (based on live CAR T cell counts, as described above), and proliferation was normalized to CAR alone control cells. As shown in FIG. 21C, proliferation was at least modestly increased in each of the TGFBR2-targeting DNA-targeting systems. In addition, DNA-targeting systems with the DNMT3A/L-XTEN80-dSpCas9-KRAB fusion protein dramatically enhanced proliferation in comparison to DNA-targeting systems with the dSpCas9-KRAB fusion protein.

### Example 8: TGFBR2 Knockdown Modulates T Cell Function

DNA-targeting systems for repression of TGF-beta receptor 2 (TGFBR2) were identified and tested for ability to modulate phenotypes in CAR T cells.

DNA-targeting systems for repression of TGFBR2 containing DNMT3A/L-XTEN80-dSpCas9-KRAB and a gRNA targeting TGFBR2 were delivered to CAR T cells by electroporation for transient expression, generally as described above in Example 5. TGFBR2-targeting gRNA target site (protospacer) sequences are set forth in SEQ ID NOS:300-302 and gRNA spacer sequences for gRNAs targeting each site are set forth in SEQ ID NOS:303-305, as shown in Table E5.A. Negative controls included cells that were CAR T cells delivered with DNMT3A/L-XTEN80-dSpCas9-KRAB (SEQ ID NO:337) and a non-targeting gRNA (NT), or cells that did not express a CAR (mock).

Cell surface expression of TGFBR2 was measured by flow cytometry at multiple time points post-electroporation with the DNA-targeting systems, and the percentage of cells negative for TFGBR2 was quantified. As shown in FIG. 21D, the DNA-targeting systems for repression of TGFBR2 led to sustained knockdown of TGFBR2 expression for up to 2 weeks post-electroporation, in comparison to negative control cells.

The CAR T cells delivered with DNA-targeting systems for repression of TGFBR2 were assessed for proliferation in the presence of titrating concentrations of TGF-beta (TGFb) ranging from 0 ng/mL to 10 ng/mL. Cell numbers of CAR T cells electroporated with the DNA-targeting systems were determined at 0 hours and then plated at 10,000 cells per well. Proliferation was monitored at 96 hours post activation with plate-bound anti-CD3/anti-CD28 T cell activation reagents to determine fold expansion. Fold expansion was normalized to 0 ng/mL TGFb conditions. As shown in FIG. 21E, negative control cells delivered with DNMT3A/L-XTEN80-dSpCas9-KRAB and a non-targeting gRNA exhibited markedly reduced proliferation upon exposure to TGFb. In contrast, DNA-targeting systems for repression of TGFBR2 facilitated proliferation of CAR T cells even in the presence of TGFb.

The CAR T cells were also assessed for secreted cytokine production when exposed to target antigen-expressing Her2 positive NCI-H1975 tumor cells in the presence of 10 ng/mL TGFb. NCI-H1975 cells were plated at 50,000 cells per well and T cells were plated at 10,000 cells per well for an effector cell to target cell ratio of 5 to 1. At 24 hours post co-culture of CAR T cells with target cells, supernatant was obtained from the wells and IFNg production was measured by MSD immunoassays, generally as described above in Example 5. As shown in FIG. 21F, in the presence of TGF-beta at a concentration of 10 ng/mL, CAR T cells electroporated with DNA-targeting systems for repression of TGFBR2 exhibited increased secreted IFNg when exposed to antigen-expressing target cells.

The CAR T cells were also assessed for secreted cytokine expression at 96 hours post activation with plate-bound anti-CD3/anti-CD28 T cell activation reagents in the presence of titrating amounts of TGFb. For each concentration of TGFb, secreted cytokine expression was normalized to expression after exposure to Ong/mL TGFb. As shown in FIG. 21G, control activated CAR T cells with a non-targeting gRNA exhibited markedly reduced secretion of IFNg, IL-2, and TNFa upon exposure to TGFb. In contrast, activated CAR T cells delivered with DNA-targeting systems for repression of TGFBR2 exhibited sustained levels of secretion of IFNg, IL-2, and TNFa upon exposure to TGFBR2 at the various concentrations.

The results support using the DNA-targeting systems for repression of TGFBR2 for driving advantageous cellular phenotypes in T cells, such as increased T cell effector function, for example in cellular therapies involving CAR T cells.

### Example 9: Transient Delivery of DNA-Targeting Systems for Activation of IL-2 or Repression of MED12 or CBLB to CAR T Cells Improves Function In Vivo

DNA-targeting systems for activation of IL-2 or repression of MED12 or CBLB were transiently delivered to CAR T cells. The CAR T cells were transplanted to a mouse model with Her2 antigen-expressing tumor cells to assess CAR T cell function in vivo.

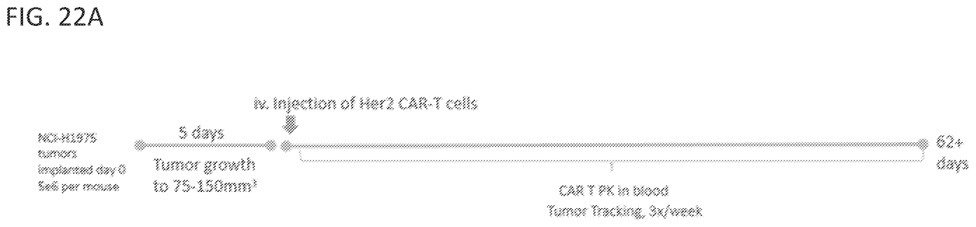

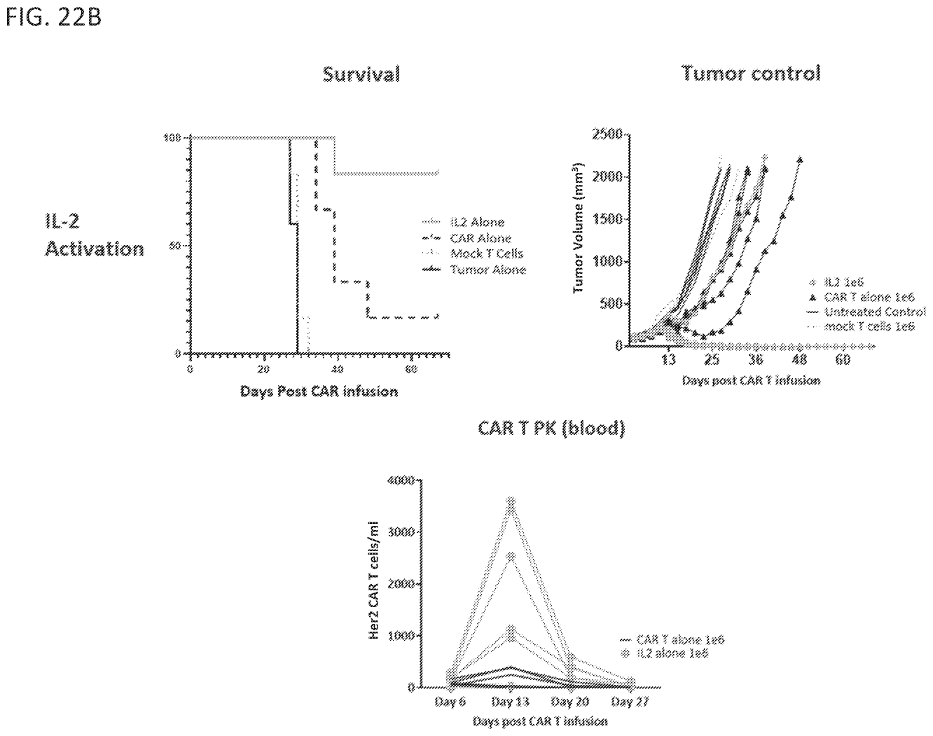

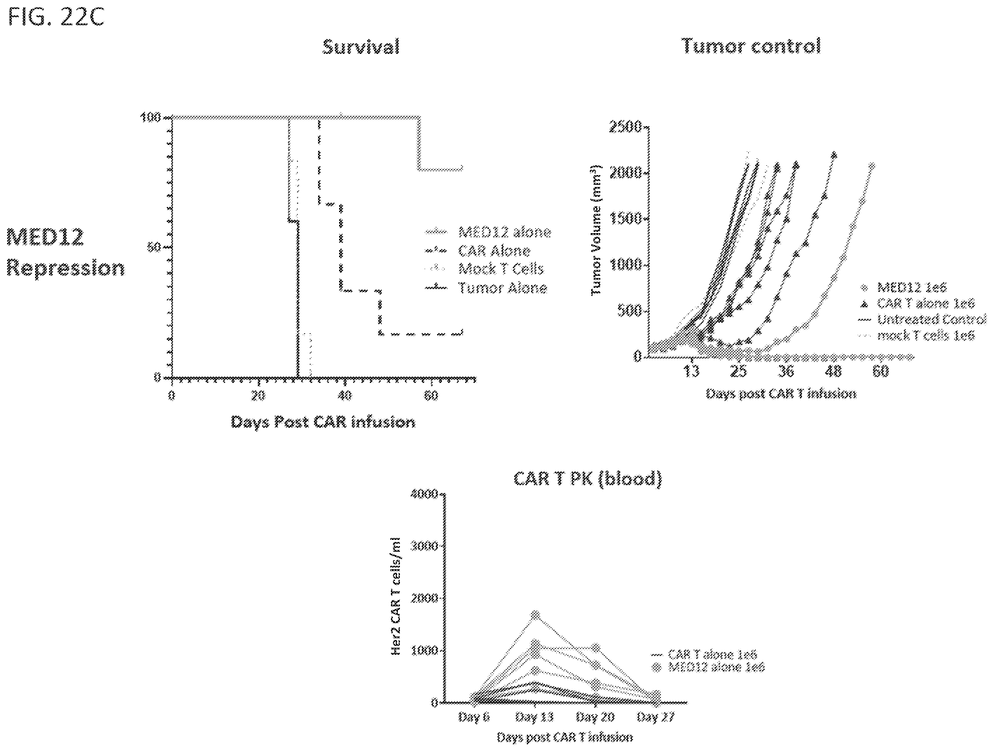

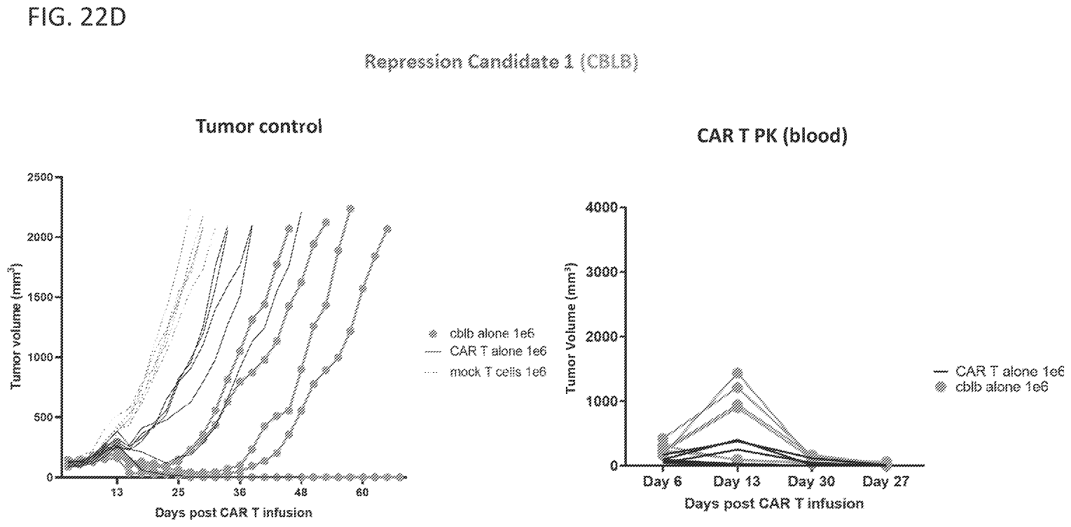

Her2 positive NCI-H1975 cells were implanted subcutaneously into the flank of NSG MHC KO mice (immunodeficient NOD scid gamma, major histocompatibility complex knockout mice). Five days after tumor implant, 1 million Her2 CAR T cells were injected intravenously into the tail vein. Experimental mice were injected with CAR T cells previously delivered with a transiently expressed DNA-targeting system for IL-2 activation (dSpCas9-2xVP64 and gRNA IL-2_1, targeting SEQ ID NO:78), CAR T cells previously delivered with a transiently expressed DNA-targeting system for MED12 repression (dSpCas9-KRAB and gRNA MED12_2, targeting SEQ ID NO:81), or CAR T cells previously delivered with a transiently expressed DNA-targeting system for CBLB repression (dSpCas9-KRAB and gRNA CBLB_2, targeting SEQ ID NO:11). Control mice were injected with CAR T cells not delivered with a DNA-targeting system (CAR alone), were injected with T cells not expressing a CAR (Mock T Cells), or were not injected with T cells (Tumor Alone). Six mice were included in each group. Mice were assessed for survival, tumor volume (measured every 2-3 days), and levels of circulating CAR T cells in the blood after T cell transfusion. The timecourse for the in vivo experiment is shown in FIG. 22A.

As shown in FIG. 22B (left), mice injected with CAR T cells delivered with the DNA-targeting system for IL-2 activation exhibited better survival than control conditions, including mice injected with CAR T cells not delivered with a DNA-targeting system (CAR Alone). As shown in FIG. 22B (middle) mice injected with CAR T cells delivered with the DNA-targeting system for IL-2 activation exhibited delayed growth or elimination of the tumor. Control mice generally exhibited rapid tumor growth. As shown in FIG. 22B (right), mice injected with CAR T cells delivered with the DNA-targeting system for IL-2 activation, exhibited higher levels of the CAR T cells than mice injected with CAR T cells not delivered with a DNA-targeting system (CAR Alone).

FIG. 22C and FIG. 22D demonstrate results in mice delivered DNA-binding systems for targeted repression of MED12 or CBLB, respectively, in the same study. Mice injected with CAR T cells delivered with the DNA-targetins system for MED12 repression exhibited delayed better survival than controls (FIG. 22C, left), delayed growth or elimination of the tumor (FIG. 22C, middle), and higher levels of CAR T cells than mice injected with CAR T cells not delivered with a DNA-targeting system (CAR Alone) (FIG. 22C, right). Similar results are shown for targeting CBLB as demonstrated by delayed growth or elimination of the tumor (FIG. 22D, left) and levels of CAR T cells (FIG. 22D, right).

The results support using the DNA-targeting systems for activation or repression of target genes for improving T cell function in vivo, including increased circulating CAR T cells, greater tumor killing, and improved survival outcomes in adoptive cell therapies with CAR T cells.

### Example 10: Enhanced Repression, Durability, and T Cell Function with Alternative Fusion Protein for Targeted Transcriptional Repression

Transiently expressed DNA-targeting systems comprising dSpCas9 fusion proteins with different transcriptional repressor effector domains were tested for ability to repress MED12 expression and modulate T cell effector function.

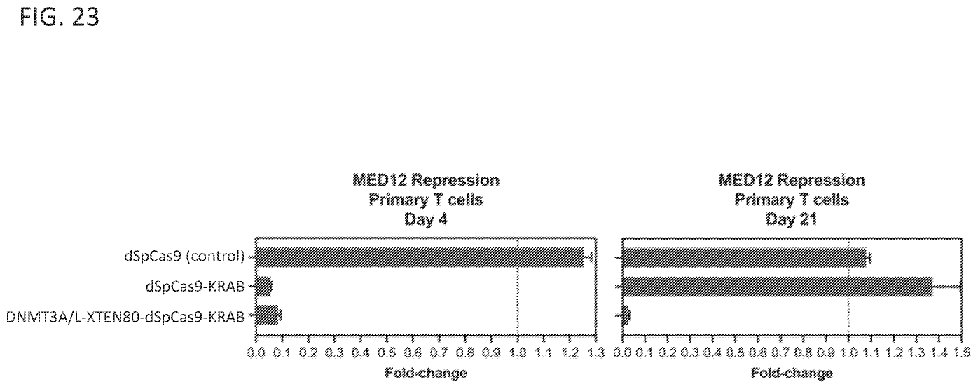

First, CD4 and CD8 T cells were thawed and activated with anti-CD3/anti-CD28 reagents. At 5 days after CD3/CD28 activation, T cells were electroporated with a MED12-targeting gRNA (MED12_2, targeting SEQ ID NO:81), and one of: a control dSpCas9 not fused to a transcriptional repressor effector domain (dSpCas9 control), a dSpCas9-KRAB fusion protein (mRNA encoding SEQ ID NO:332), or DNMT3A/L-XTEN80-dSpCas9-KRAB (mRNA encoding SEQ ID NO:337). Electroporated mRNAs encoding the dSpCas9 fusion proteins further encoded an N-terminal FLAG epitope (SEQ ID NO:364) and a C-terminal P2A-mCherry domain (SEQ ID NO:354) for assessing expression of the transduced fusion proteins. The full mRNA for the dSpCas9-KRAB encoded SEQ ID NO:312, and the full mRNA for the DNMT3A/L-XTEN80-dSpCas9-KRAB encoded SEQ ID NO:317. Expression of MED12 was assessed by RT-qPCR at day 4 and day 21 post-electroporation with the DNA-targeting systems, and expression levels were normalized to expression levels in T cells electroporated with the same fusion protein but with a non-targeting gRNA. As shown in FIG. 23, DNA-targeting systems with both dSpCas9-KRAB and DNMT3A/L-XTEN80-dSpCas9-KRAB fusion proteins induced strong repression of MED12 at 4 days post-EP in comparison to dSpCas9 condition, with MED12 expression being reduced by greater than 90% with both fusion proteins. However, at day 21 post-EP, cells delivered with dSpCas9-KRAB and gRNA MED12_2 recovered full MED12 expression in comparison to controls. In contrast, cells delivered with DNMT3A/L-XTEN80-dSpCas9-KRAB and gRNA MED12_2 exhibited sustained repression at greater than 90% knockdown in comparison to controls. The results showed that the transiently expressed DNA-targeting system with DNMT3A/L-XTEN80-dSpCas9-KRAB and MED12-targeting gRNA mediated lasting epigenetic effects as evidenced by robust and sustained repression.

CD4 and CD8 T cells were thawed and activated with anti-CD3/anti-CD28 reagents, and transduced with a Her2 CAR at 24 hours post-activation. Mock T cells not transduced with the Her2 CAR were also included as control cells. At four days after transduction with the Her2 CAR, the CAR T cells were electroporated with DNA-targeting systems containing a MED12-targeting gRNA (selected from: MED12_2 targeting SEQ ID NO:81; MED12_3 targeting SEQ ID NO:82, MED12_4 targeting SEQ ID NO:83, and MED12_7 targeting SEQ ID NO:86), and with mRNA encoding either a dSpCas9-KRAB fusion protein (encoding SEQ ID NO:332) or a DNMT3A/L-XTEN80-dSpCas9-KRAB fusion protein (encoding SEQ ID NO:337). mRNAs encoding the dSpCas9 fusion proteins further comprised an N-terminal FLAG epitope (SEQ ID NO:364) and a C-terminal P2A-mCherry domain (SEQ ID NO:354) for assessing expression of the transduced fusion proteins. The full mRNA for the dSpCas9-KRAB encoded SEQ ID NO:312, and the full mRNA for DNMT3A/L-XTEN80-dSpCas9-KRAB encoded SEQ ID NO:317. Control cells included CAR T cells electroporated with DNMT3A/L-XTEN80-dSpCas9-KRAB alone, CAR T cells electroporated with DNMT3A/L-XTEN80-dSpCas9-KRAB and a non-targeting gRNA (NTg), or T cells not expressing a CAR (Mock). Cells were analyzed at various time points post-electroporation (post-EP) with the DNA-targeting systems to assess targeted gene repression and T cell effector function phenotypes.

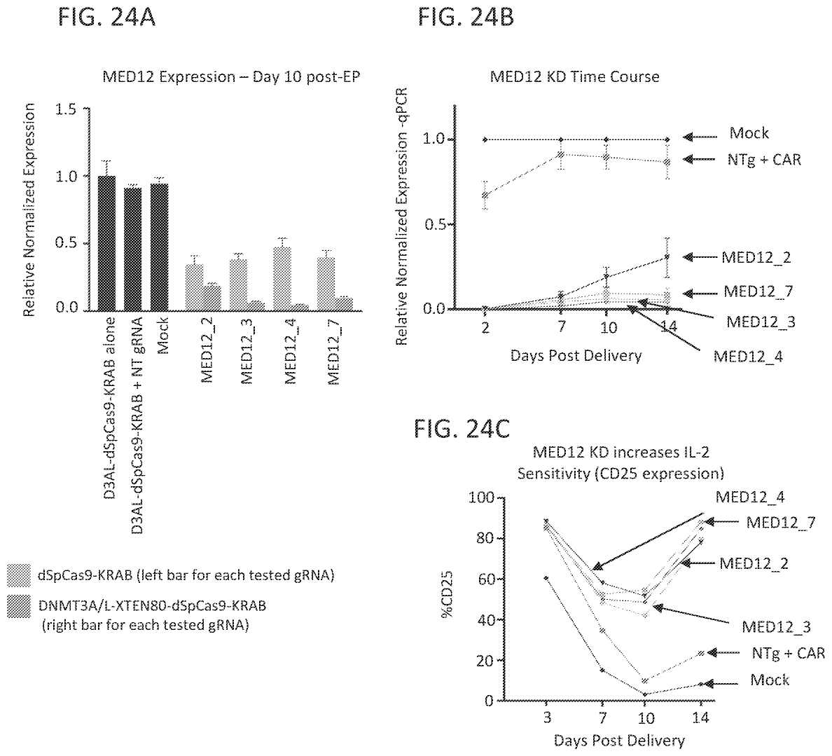

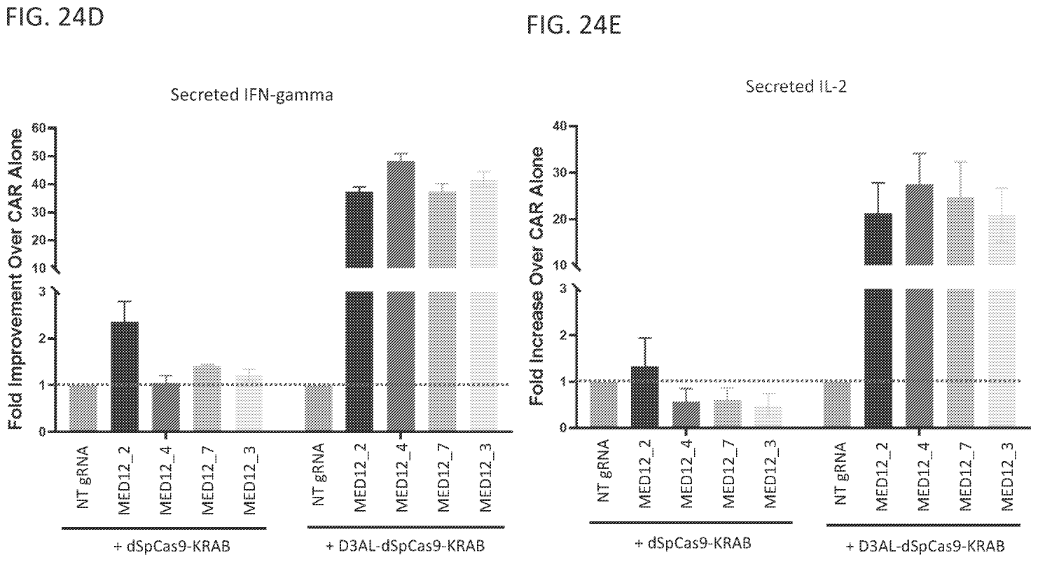

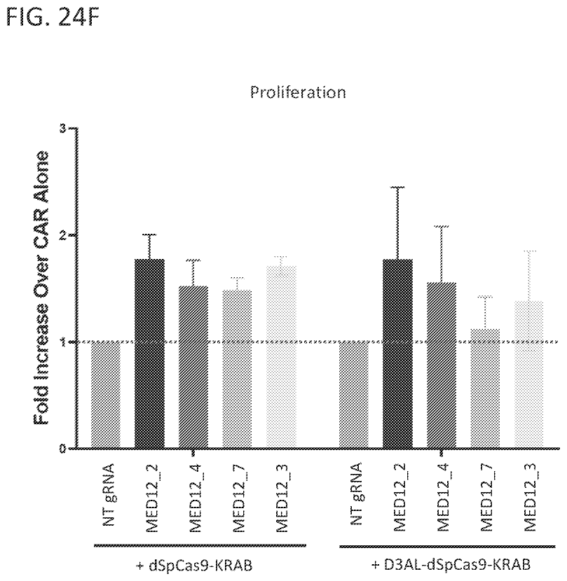

First, knockdown of MED12 expression was measured by qRT-PCR. FIG. 24A shows MED12 expression at day 10 post-electroporation. As shown in the figure, dSpCas9-KRAB and DNMT3A/L-XTEN80-dSpCas9-KRAB both mediated knockdown of MED12 repression with the various indicated MED12 gRNAs, which was sustained at least until Day 10. DNMT3A/L-XTEN80-dSpCas9-KRAB mediated stronger repression of MED12 at day 10 than dSpCas9-KRAB, including with each of the four tested MED12-targeting gRNAs. The results showed that the DNA-targeting systems induced sustained transcriptional repression of targeted genes, and that DNMT3A/L-XTEN80-dSpCas9-KRAB mediated an enhanced effect.

MED12 expression was measured in the CAR T cells electroporated with DNMT3A/L-XTEN80-dSpCas9-KRAB and MED12-targeting gRNAs, in comparison to control cells, at 2, 7, 10, and 14 days post-delivery. As shown in FIG. 24B, MED12 expression was strongly repressed for at least 14 days post-delivery including with each of the MED12-targeting gRNAs. With 3 of the 4 tested gRNAs, MED12 expression was reduced to at or about 10% or less of control levels at day 14.

CD25 cell surface expression, a measure of IL-2 sensitivity, was also measured by flow cytometry in the CAR T cells electroporated with DNMT3A/L-XTEN80-dSpCas9-KRAB and MED12-targeting gRNAs, in comparison to control cells, at 2, 7, 10, and 14 days post-delivery. As shown in FIG. 24C, MED12 repression with the transiently expressed DNA-targeting systems led to increased CD25 expression, including a dramatic increase in CD25 expression at 2 weeks post-EP in comparison to control cells.

The CAR T cells electroporated with the transiently expressed DNA-targeting systems containing dSpCas9-KRAB or DNMT3A/L-XTEN80-dSpCas9-KRAB and one of the MED12-targeting gRNAs were serially stimulated with Her2-positive NCI H1975 tumor cells at a ratio of 1:5 CAR T:tumor cells, with serial stimulations occurring 4 days apart. 24 hours after the second stimulation, secreted IFN-gamma and IL-2 were measured by MSD immunoassay (as described above), normalized to CAR T cells electroporated with a non-targeting gRNA (CAR NTg). CAR T cell proliferation was measured after the second stimulation (based on live CAR T cell counts, as described above), and was also normalized to CAR T cells electroporated with a non-targeting gRNA (CAR NTg). As shown in FIG. 24D, dSpCas9-KRAB with gRNA MED12_2 increased secreted IFNg in the CAR T cells by approximately 2-fold at the assayed timepoint. MED12-targeting systems with DNMT3A/L-XTEN80-dSpCas9-KRAB dramatically increased secreted IFNg in comparison to control cells, by a factor of 30-fold to 50-fold, with all four tested MED12-targeting gRNAs, demonstrating an enhanced effect on CAR T cell effector function in comparison to the DNA-targeting systems with dSpCas9-KRAB. As shown in FIG. 24E, DNA-targeting systems with DNMT3A/L-XTEN80-dSpCas9-KRAB also dramatically increased secreted IL-2 by 20-fold to 30-fold in comparison to control cells with all four tested MED12-targeting gRNAs. IL-2 secretion was also enhanced in comparison to cells delivered with DNA-targeting systems with dSpCas9-KRAB. As shown in FIG. 24F, CAR T cells delivered with DNA-targeting systems with both dSpCas9-KRAB and DNMT3A/L-XTEN80-dSpCas9-KRAB exhibited increased proliferation in comparison to control cells by up to 2-fold increased proliferation.

### Example 11: Transient Expression of DNA-Targeting Systems

An exemplary DNA-targeting system was delivered by electroporation to T cells in accordance with the methods provided herein, and the transient nature of expression of the DNA-targeting system was assessed.

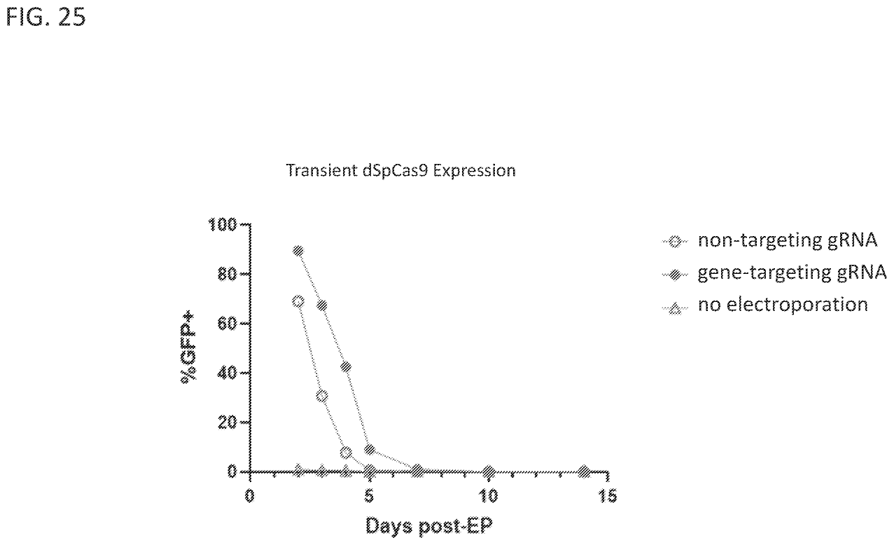

FIG. 25 demonstrates transient expression in T cells of an exemplary dSpCas9 protein following delivery by electroporation of mRNA encoding the protein and a non-targeting gRNA or gene-targeting gRNA, in accordance with the transient delivery methods used herein and as described the above Examples. The mRNA encoding the dSpCas9 further comprised a sequence encoding GFP, which was used as a surrogate to detect expression of the dSpCas9 protein in electroporated cells. The cells were assessed at timepoints following electroporation by flow cytometry to determine the percentage of cells expressing the dSpCas9 protein (i.e. % GFP+ cells). Control cells were not electroporated. FIG. 25 shows robust expression of the dSpCas9 protein at 3 days post-electroporation, followed by a decline to no detectable dSpCas9 expression after Day 7 post-electroporation. These results confirm transient expression of the DNA-targeting systems described herein, and suggest that the resulting enduring phenotypes result from sustained and/or heritable epigenetic effects in dividing T cells, including gene repression and T cell effector function.

### Example 12: Identification of Fusion Proteins for Targeted Transcriptional Repression

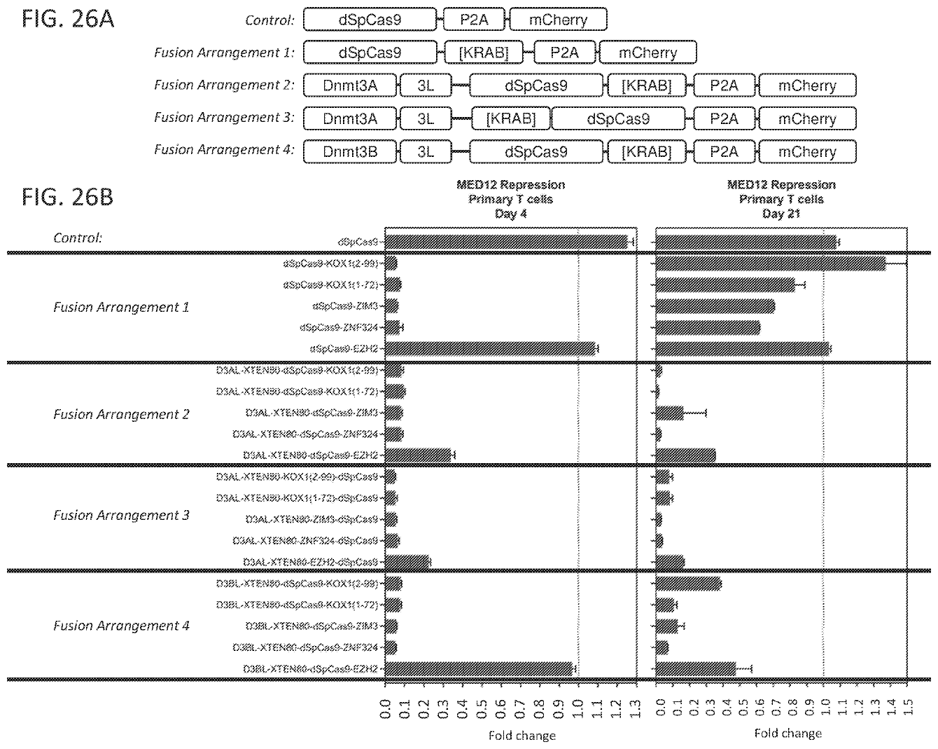

Fusion proteins comprising dSpCas9 and transcriptional repressor effector domains in different arrangements and combinations were tested for ability to repress and maintain repression of gene expression when delivered with a gene-targeting gRNA. [0716]20 different dSpCas9 fusion proteins for transcriptional repression were designed. The 20 different dSpCas9 fusion proteins comprised four general arrangements of components, shown as Fusion Arrangement 1 through 4, in FIG. 26A. In the first arrangement (Fusion Arrangement 1), the dSpCas9 fusion protein comprised from N-terminus to C-terminus: dSpCas9, and a variable repression domain. In Fusion Arrangement 2, the dSpCas9 fusion protein comprised from N-terminus to C-terminus: a DNMT3A domain, a DNMT3L domain, an XTEN80 linker, dSpCas9, and a variable repression domain. In Fusion Arrangement 3, the dSpCas9 fusion protein comprised from N-terminus to C-terminus: a DNMT3A domain, a DNMT3L domain, an XTEN80 linker, a variable repression domain, and dSpCas9. In Fusion Arrangement 4, the dSpCas9 fusion protein comprised from N-terminus to C-terminus: a DNMT3B domain, a DNMT3L domain, an XTEN80 linker, dSpCas9, and a variable repression domain. For each of the 4 Fusion Arrangements, 5 different fusion proteins were designed, with each fusion protein comprising a different variable repression domain, selected from a KRAB domain from KOX1 (KOX1(2-99)) (SEQ ID NO:355; KRAB domain used in dSpCas9 fusion proteins of preceding Examples), a KRAB domain from KOX1 (KOX1(1-72)) (SEQ ID NO:356), a KRAB domain from ZIM3 (SEQ ID NO:357), a KRAB domain from ZNF324 (SEQ ID NO:358), and an EZH2 domain (SEQ ID NO:359). Fusion proteins further comprised components such as linkers and NLS sequences, including those shown in Table E12. mRNA encoding each of the fusion proteins was prepared for transient delivery by electroporation, as described above, with mRNA further encoding an N-terminal FLAG epitope (SEQ ID NO:364) and a C-terminal P2A-mCherry domain (SEQ ID NO:354) for assessing expression of the transduced fusion proteins. SEQ ID NOs of the fusion protein sequences without, and with the FLAG and P2A-mCherry domains are shown in Table E11 below. Various components of the fusion proteins from Table E11 are shown in Table E12.

TABLE E11
Fusion proteins for transcriptional repression

| | | SEQ ID NO of Full Fusion |
|---|---|---|
| | | Protein encoded by mRNA |
| | SEQ ID NO of | including N-terminal FLAG |
| Fusion Protein | Fusion Protein | and C-terminal P2A-mCherry |
| dSpCas9-KOX1(2-99) | 332 | 312 |
| (dSpCas9-KRAB) | | |
| dSpCas9-KOX1(1-72) | 333 | 313 |
| dSpCas9-ZIM3 | 334 | 314 |
| dSpCas9-ZNF324 | 335 | 315 |
| dSpCas9-EZH2 | 336 | 316 |
| D3AL-XTEN80-dSpCas9-KOX1(2-99) | 337 | 317 |
| (DNMT3A/L-XTEN80-dSpCas9-KRAB) | | |
| D3AL-XTEN80-dSpCas9-KOX1(1-72) | 338 | 318 |
| D3AL-XTEN80-dSpCas9-ZIM3 | 339 | 319 |
| D3AL-XTEN80-dSpCas9-ZNF324 | 340 | 320 |
| D3AL-XTEN80-dSpCas9-EZH2 | 341 | 321 |
| D3AL-XTEN80-KOX1(2-99)-dSpCas9 | 342 | 322 |
| D3AL-XTEN80-KOX1(1-72)-dSpCas9 | 343 | 323 |
| D3AL-XTEN80-ZIM3-dSpCas9 | 344 | 324 |
| D3AL-XTEN80-ZNF324-dSpCas9 | 345 | 325 |
| D3AL-XTEN80-EZH2-dSpCas9 | 346 | 326 |
| D3BL-XTEN80-dSpCas9-KOX1(2-99) | 347 | 327 |
| D3BL-XTEN80-dSpCas9-KOX1(1-72) | 348 | 328 |
| D3BL-XTEN80-dSpCas9-ZIM3 | 349 | 329 |
| D3BL-XTEN80-dSpCas9-ZNF324 | 350 | 330 |
| D3BL-XTEN80-dSpCas9-EZH2 | 351 | 331 |

TABLE E12
Components of Fusion Proteins from Table E11/FIG. 26A

| Fusion Protein Component | SEQ ID NO: |
|---|---|
| dSpCas9 | 127 |
| KRAB domain from KOX1 (2-99) | 355 |
| KRAB domain from KOX1 (1-72) | 356 |
| KRAB domain from ZIM3 | 357 |
| KRAB domain from ZNF324 | 358 |
| EZH2 domain | 359 |
| DNMT3A domain | 238 |
| DNMT3B domain | 360 |
| DNMT3L domain | 241 |
| Linker - 20AA | 362 |
| Linker - 27AA | 243 |
| Linker - XTEN80 | 361 |
| SV40 NLS | 269 |
| nucleoplasmin NLS | 270 |
| FLAG | 364 |
| P2A | 352 |
| mCherry | 353 |

CD4 and CD8 T cells were thawed and activated with anti-CD3/anti-CD28 reagents. 5 days after T cell activation, cells were electroporated with DNA-targeting systems comprising a MED12-targeting gRNA (MED12_2, targeting SEQ ID NO:81), and the different dSpCas9 fusion proteins with different repressor domains. The fusion proteins were delivered as mRNA encoding the fusion proteins, with mRNAs further encoding P2A-mCherry to confirm expression of the fusion proteins in the electroporated T cells. Control cells were electroporated with mRNA encoding dSpCas9 without a transcriptional repressor effector domain. At day 4 and day 20 post-electroporation with the DNA-targeting systems, MED12 expression was assessed by RT-qPCR. For each experimental condition, results were normalized to expression levels in T cells electroporated with the same fusion protein but with a non-targeting gRNA.

As shown in FIG. 26B, as expected, dSpCas9 alone did not repress the targeted gene MED12, whereas the dSpCas9-KRAB fusion protein tested in the previous examples (having KRAB domain KOX1(2-99)) robustly repressed expression of MED12 at 4 days post-electroporation. Several of the other dSpCas9 fusion proteins with different Fusion Arrangements and variable repression domains also repressed expression of MED12 at day 4. Cells electroporated with dSpCas9-KRAB(KOX1(2-99)) did not exhibit sustained repression of MED12 at day 21. In contrast, several of the dSpCas9 fusion proteins induced sustained repression of MED12 at day 21 post-EP.

Taken together, the results show that the exemplary DNA-targeting systems comprising dSpCas9 transcriptional repressor effector domain fusion proteins are capable of inducing heritable epigenetic modifications, including gene repression, to modulate T cell effector function in a durable manner.

### Example 13: Stable Gene Repression and T Cell Phenotype Modulation Following Transient Delivery of DNA-Targeting Systems for Repression of TGFBR2, MED12, CISH, or Combinations Thereof

DNA-targeting systems for individual or multiplexed gene repression were assessed for ability to induce gene repression and improved T cell effector function after serial stimulations under immunosuppressive conditions. The DNA-targeting systems contained gRNAs or combinations thereof identified in the previous Examples.

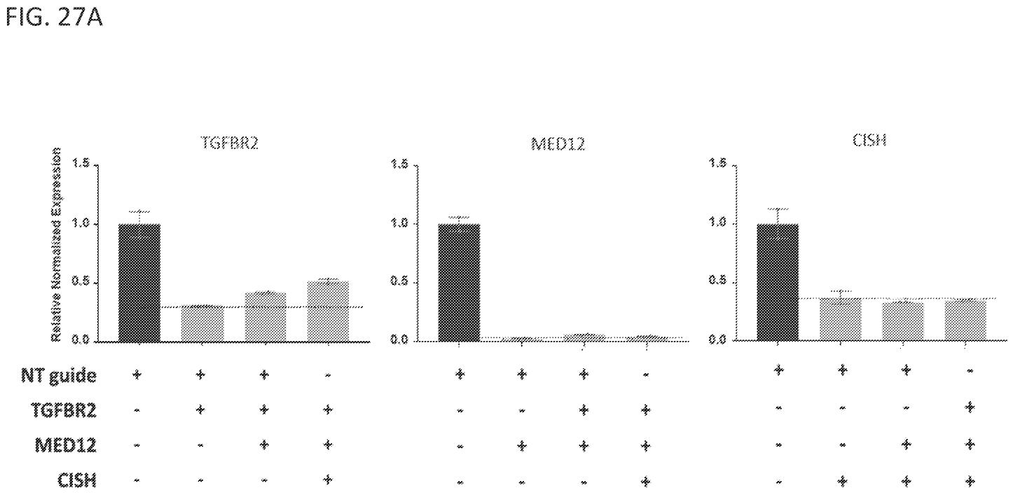

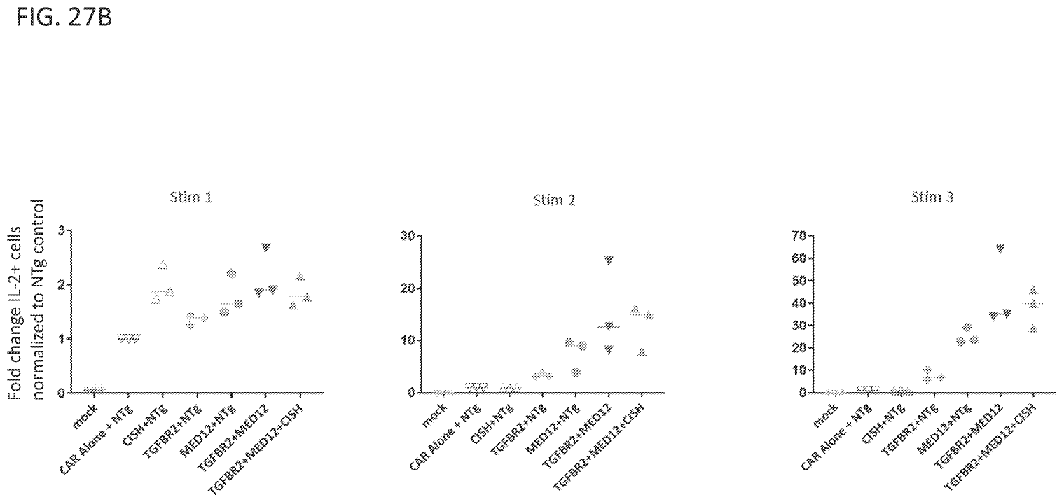

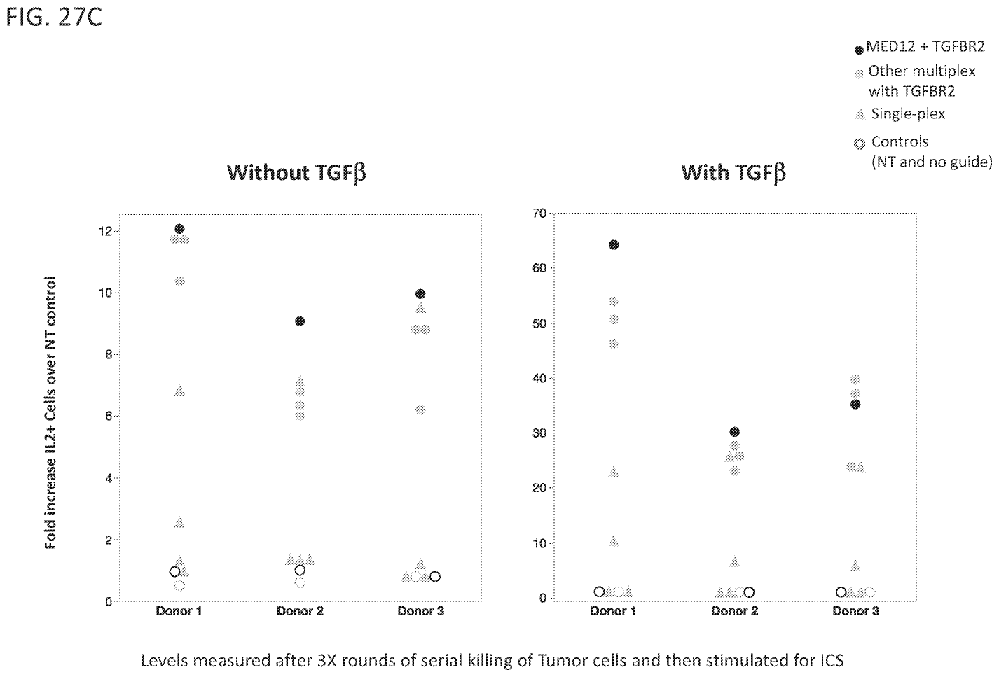

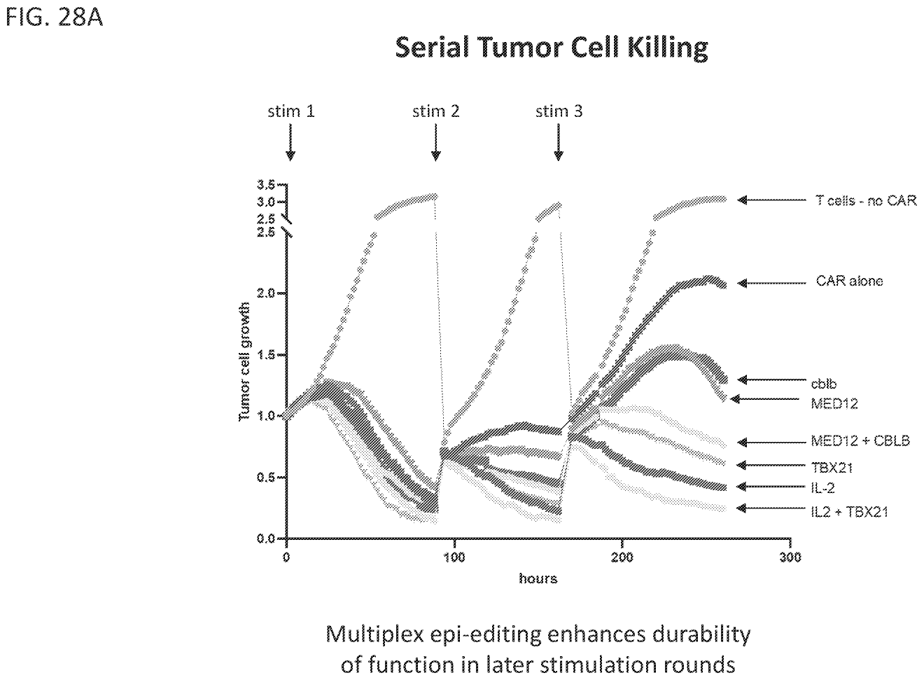

DNA-targeting systems containing a dSpCas9 fusion protein for transcriptional repression (e.g. dSpCas9-KRAB-DNMT3A/L, for example as set forth in SEQ ID NO:75) and a single gRNA or combinations of gRNAs targeting TGFBR2 (gRNA TGFBR2_2, targeting SEQ ID NO:301), MED12 (gRNA MED12_3, targeting SEQ ID NO:82), and/or CISH (gRNA CISH_1, targeting SEQ ID NO:28) were transiently expressed in Her2 CAR T cells. The gRNAs further included a scaffold sequence for recruiting SpCas9 (e.g. as set forth in SEQ ID NO:69). Control cells were delivered with the dSpCas9 fusion protein and a non-targeting gRNA (“NT guide”). At 72 hours post-delivery, expression of TGFBR2, MED12, and CISH was assessed by RT-qPCR, with results normalized to comparable CAR T cells not delivered with a DNA-targeting system. As shown in FIG. 27A, DNA-targeting systems individually targeting TGFBR2, MED12, or CISH effectively repressed the respective target gene. Furthermore, multiplexed DNA-targeting systems targeting combinations of TGFBR2, MED12, and CISH also effectively repressed each targeted gene.

Next, Her2 CAR T cells were delivered with DNA-targeting systems for individual or multiplexed repression of TGFBR2, MED12, and/or CISH, which included a fusion protein for repression (e.g. dSpCas9-KRAB-DNMT3A/L, for example as set forth in SEQ ID NO:75) and gRNAs targeting the indicated genes (TGFBR2_2, targeting SEQ ID NO:301; MED12_3, targeting SEQ ID NO:82; CISH_1, targeting SEQ ID NO:28). The gRNAs further included a scaffold sequence for recruiting SpCas9 (e.g. as set forth in SEQ ID NO:69). The DNA-targeting systems were delivered as mRNA encoding the fusion protein and pre-transcribed gRNAs. For conditions in which a single gene was targeted, a non-targeting gRNA was included (“NTg”) to achieve molar equivalency of gRNA:fusion protein ratio across samples. Control cells did not express a CAR (“mock”), or were not delivered with a gene-targeting gRNA (“CAR Alone+NTg”).

After delivery of the DNA-targeting systems, cells were subjected to serial stimulation (3 stimulations, each 4 days apart) with Her2-expressing tumor cells. To mimic immune suppression, 2.5 ng/mL of recombinant human TGF-beta was added to the culture. Cells were assessed for IL-2 expression by ICS and flow cytometry after each of the 3 stimulations (Stim 1, Stim 2, and Stim 3). IL-2 expression was measured as the percentage of IL-2+ viable T cells, and quantified as fold-change over control cells delivered with a non-targeting gRNA. As shown in FIG. 27B, multiple of the DNA-targeting systems for individual or multiplexed repression of TGFBR2, MED12, and/or CISH increased IL-2 expression in a durable manner, including after multiple stimulations and under immunosuppressive conditions.

In another experiment, multiplexed repression by delivering CAR-T cells a fusion protein for repressiondSpCas9-KRAB-DNMT3A/L and a gRNA targeting TGFBR2 (TGFBR2_2, targeting SEQ ID NO:301) and another gene (MED12_3, targeting SEQ ID NO:82; CISH_1, targeting SEQ ID NO:28; or CBLB_2, targeting SEQ ID NO: 11) consistently increased IL-2 expression in both the presence and absence of recombinant human TGF-beta, compared to systems for single-plex targeting of a single gene (FIG. 27C). In particular, multiplexed DNA-binding systems for repression of TGFBR2 and MED12 exhibited among the most superior ability to increase IL-2 in cells among 3 different donors, in the presence or absence of TGF-beta. These data were generated using T cells from three donors and demonstrate the profound effects of multiplexed repression on CAR T cell activity.

The results support the utility of the DNA-targeting systems provided herein for repressing target genes and improving T cell effector functions, including under immunosuppressive conditions. The results also further demonstrate that simultaneous epigenetic modifications can create synergy and expand options for driving advantageous cell phenotypes, for example in ACT therapies and other therapies.

### Example 14: Effects on Tumor Cell Killing of Combined IL-2 and TBX21 Activation or Combined MED12 and CBLB Repression

DNA-targeting systems for combined IL-2 and TBX21 activation or for combined MED12 and CBLB repression were tested for their effects on tumor cell killing by modified T cells. To measure the ability of CAR T cells to kill target cells (i.e., cytotoxic activity), target cells were tracked in co-culture with CAR T cells using an Incucyte automated imaging system. Her2-expressing, NCI-H1975 (ATCC CRL-5908) target cells were engineered to express a fluorescent tag (Nuclight Red), allowing imaging and quantification of target cells over time to generate a growth curve for the target cells. Reduced fluorescence over time was indicative of decreased target cell numbers and increased killing.

For epiediting experiments to activate genes, Her2 CAR T cells were delivered mRNA encoding a dSpCas9-2xVP64 effector fusion protein (SEQ ID NO:76, encoding SEQ ID NO:77) and one or both of an SpCas9 IL-2-targeting gRNA (IL2_1, targeting SEQ ID NO:78) and an SpCas9 TBX21-targeting gRNA (TBX21_3, targeting SEQ ID NO: 155). The gRNAs further comprised an SpCas9 scaffold sequence (SEQ ID NO: 69). A non-targeting gRNA was added as needed to maintain molar equivalency of guide:effector ratio across samples.

For epiediting to repress genes, Her2 CAR T cells were delivered mRNA encoding a dSpCas9-KRAB-DNMT3A/L effector fusion protein (for example as set forth in SEQ ID NO:75) and one or both of an SpCas9 MED12-targeting gRNA (MED12_3, targeting SEQ ID NO:82) and an SpCas9 CBLB-targeting gRNA (CBLB_2, targeting SEQ ID NO: 11). The gRNAs further comprised an SpCas9 scaffold sequence (SEQ ID NO: 69). A non-targeting gRNA was added as needed to maintain molar equivalency of guide:effector ratio across samples.

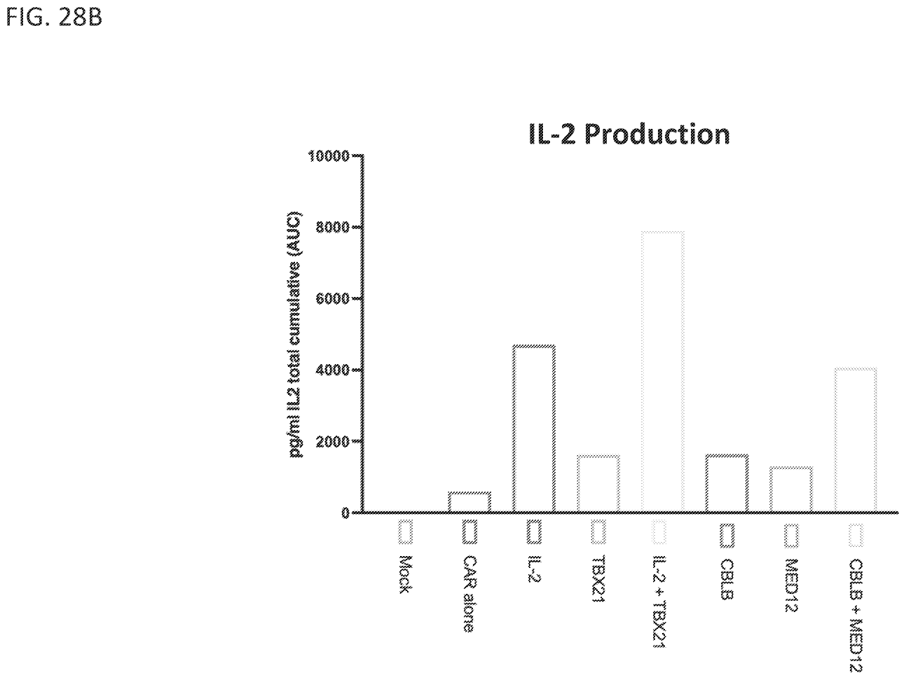

Three days following delivery of the DNA-targeting system, CAR T cells were co-cultured with target cells and monitored. Serial stimulations (shown as stim 1, stim 2, and stim 3 in FIG. 28A) were performed by replating CAR T cells with fresh target cells at a 1:4 ratio of CAR T cells:target cells. As shown in FIG. 28A, combined IL-2 and TBX21 activation or combined MED12 and CBLB repression led to increased tumor cell killing as compared to the CAR-only group and to groups receiving only one of the gRNAs. These data show that multiplexed epi-editing enhanced durability of function in the later stimulation rounds.

As shown in FIG. 28B, combined IL-2 and TBX21 activation or combined MED12 and CBLB repression led to an increase in IL-2 production as compared to the CAR-only group and to groups receiving only one of the gRNAs.

Together, these data demonstrate that simultaneous activation of target genes or simultaneous repression of target genes creates synergy and enhances capacity for cell phenotype control.

### Example 15: Transient Delivery of Multiplexed DNA-Targeting Systems Improves CAR T Function In Vivo

DNA-targeting systems for modulation of different target genes transiently delivered to CAR T cells were tested for their effect on in vivo CAR T cell activity. The CAR T cells were transplanted to a mouse model with Her2 antigen-expressing tumor cells to assess CAR T cell function in vivo using the model as described in Example 9.

Her2 positive NCI-H1975 cells were implanted subcutaneously into the flank of NSG MHC KO mice (immunodeficient NOD scid gamma, major histocompatibility complex knockout mice). Five days after tumor implant, 1×10⁶Her2 CAR T cells (high dose) or 0.3×10⁶ Her2 CAR T cells (low dose) were injected intravenously into the tail vein.

For epiediting experiments to activate genes, experimental mice were injected with CAR T cells previously delivered with a transiently expressed DNA-targeting systems for IL-2 activation (dSpCas9-2xVP64 and gRNA IL-21, targeting SEQ ID NO:78), individually or in combination with DNA-targeting systems for LCP2 (dSpCas9-2xVP64 and gRNA LCP2_2, targeting SEQ ID NO:151), EOMES (dSpCas9-2xVP64 and gRNA EOMES_3, targeting SEQ ID NO:149), TBX21 (dSpCas9-2xVP64 and gRNA TBX21_3, targeting SEQ ID NO:155). For epiediting experiments to repress genes, experimental mice were injected with CAR T cells delivered with a transiently expressed DNA-targeting systems for MED12 repression (dSpCas9-KRAB and gRNA MED12_2, targeting SEQ ID NO:81), CBLB (dSpCas9-KRAB and gRNA CBLB_2, targeting SEQ ID NO: 11), or CISH_1 (dSpCas9-KRAB and gRNA CISH_1, targeting SEQ ID NO: 28) individually or in combination with DNA-targeting systems for CBLB or CISH.

Control mice were injected with CAR T cells not delivered with a DNA-targeting system (CAR alone), were injected with T cells not expressing a CAR (Mock T Cells), or were not injected with T cells (Tumor Alone). Six mice were included in each group. Mice were assessed tumor volume (measured every 2-3 days) and levels of circulating CAR T cells in the blood after T cell transfusion.

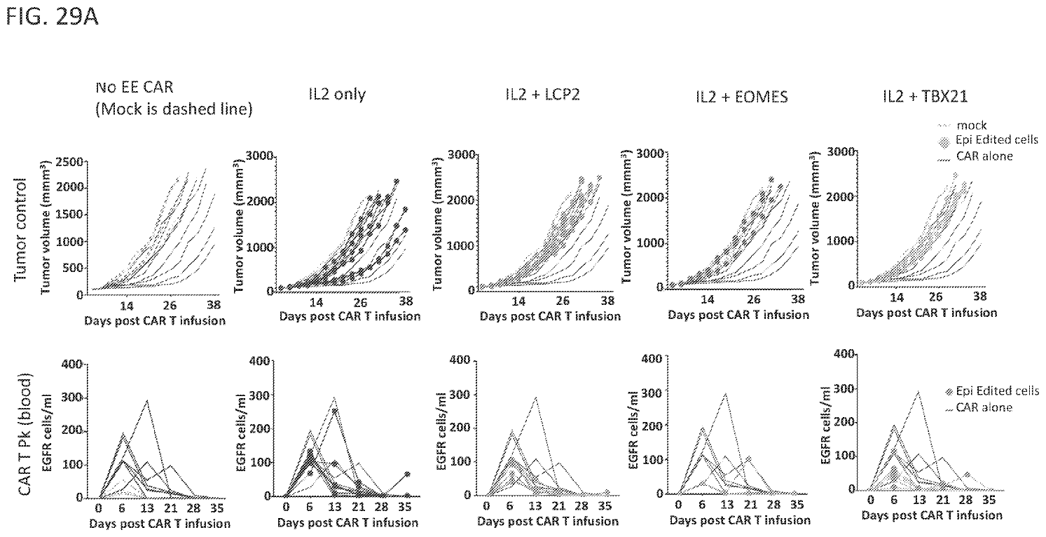

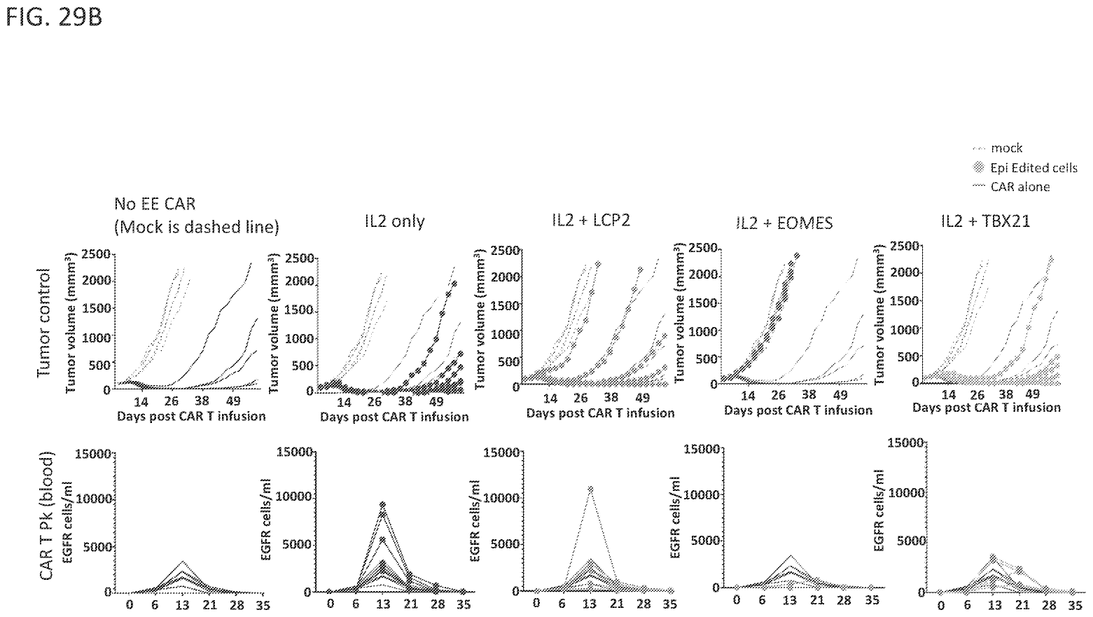

Results for anti-tumor function and pharmacokinetics of administered CAR T cells transiently delivered with epieditors for activation of genes is shown in FIG. 29A (low dose) and FIG. 29B (high dose). As shown, the greatest reduction in tumor control and increased CAR-T cells in blood was seen in mice delivered CAR T cells treated with an epieditor targeting only IL-2, whereas further combined targeting with LCP2, EOMES and TBX21 did not further increase CAR T cell activity in this study (FIG. 29A and FIG. 29B). As shown in FIG. 29B, the pharmacokinetics revealed slightly higher levels of the CAR T cells than mice injected with CAR T cells not delivered with a DNA-targeting system (CAR Alone), particularly for mice delivered CAR T cells treated with an epieditor targeting only IL-2.

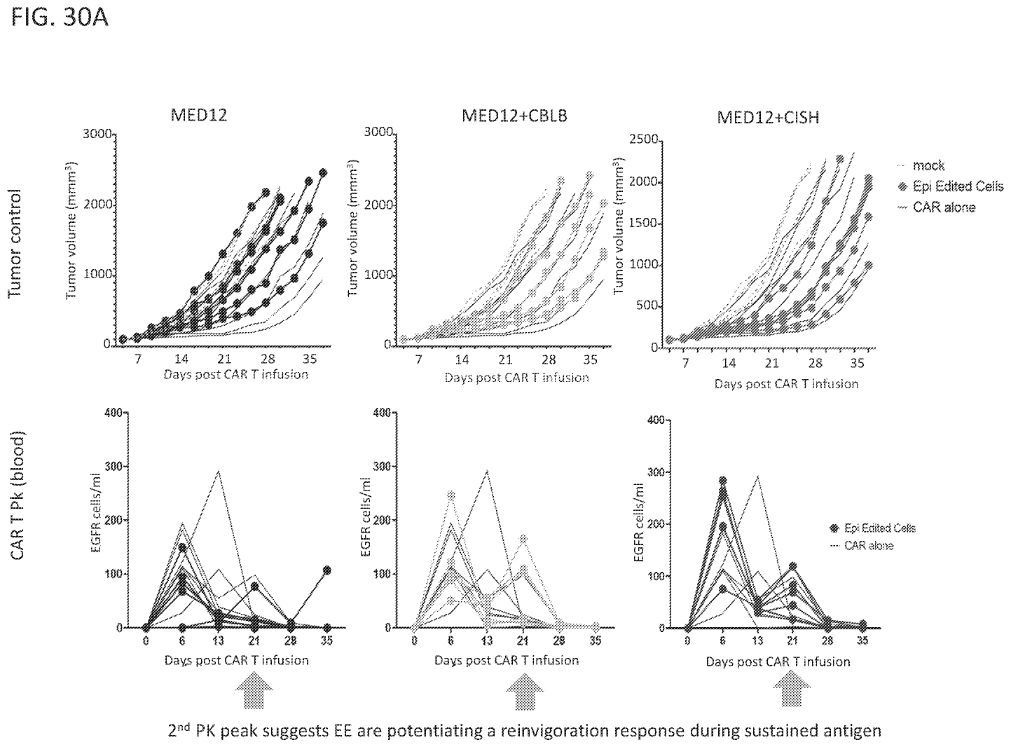

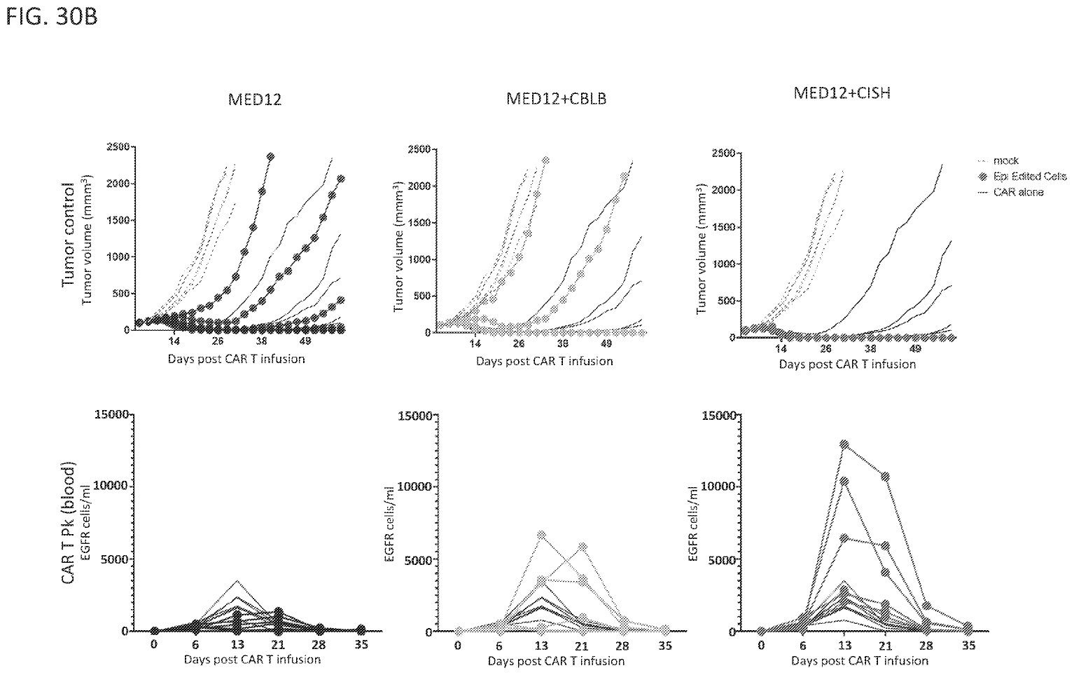

Results for anti-tumor function and pharmacokinetics of administered CAR T cells transiently delivered with epieditors for repression of genes is shown in FIG. 30A (low dose) and FIG. 30B (high dose). As shown in FIG. 30A, mice injected with low CAR T cell dose delivered with the multiplexed DNA-targeting systems for MED12, CBLB, and CISH repression revealed delayed growth of tumors and increased CAR-T cells in the blood. In particular, the pharmacokinetics plots shown in FIG. 30A (bottom) revealed second peaks suggesting that the epiedited CAR T cells potentiated a reinvigoration response. Pharmacokinetic results in FIG. 30B (bottom) demonstrated higher levels of the CAR T cells in mice in which CAR T cells had been transiently delivered with DNA targeting systems targeting MED12+CBLB and MED12+CISH combinations, than mice injected with CAR T cells not delivered with a DNA-targeting system (CAR Alone).

The results support using the DNA-targeting systems for activation or repression of target genes for improving T cell function in vivo, including increased circulating CAR T cells, greater tumor killing, and improved survival outcomes in adoptive cell therapies with CAR T cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO | Sequence | Description |
| 1 | TTGCACTGGAAGGGTAAAGC | CD5_1 target site |
| 2 | GGAGGCGACCAAGTAAAGGC | CD5_2 target site |
| 3 | GTCAGTGGGGGACCTCGCAG | CD5_3 target site |
| 4 | CGCGCGGGCAGCGTGAAGCG | KDM1A_1 target |
| | | site |
| 5 | AGCGGCAGCAACCGGGACGG | KDM1A_2 target |
| | | site |
| 6 | GCCCAGAAGCCCTAAGACCA | KDM1A_3 target |
| | | site |
| 7 | TGTCGCTCCACAGGCGAGCA | VAV1_1 target site |
| 8 | CCTCTCAGGGCGACAGTTAC | VAV1_2 target site |
| 9 | GGCCAGCTAGACTATGAGAT | VAV1_3 target site |
| 10 | GAACAGCTCGCTCCCGAAGA | CBLB_1 target site |
| 11 | CGCTGGGTTGCTCCTTCTTC | CBLB_2 target site |
| 12 | CGTCCAGGCAGACGGCGGTG | CBLB_3 target site |
| 13 | GGGACACGGGCGGGATCGGT | DGKZ_1 target |
| | | site |
| 14 | GGAGCGAGCGCGCGCCATGG | DGKZ_2 target |
| | | site |
| 15 | TCTTCGGGCACAGGTGAGCG | DGKZ_3 target |
| | | site |
| 16 | GCCGAATGGGAGCGGCGACC | MYB_1 target site |
| 17 | GGATCCCTCGCCGACACCCG | MYB_2 target site |
| 18 | GAAACTTCGCCCCAGCGGTG | MYB_3 target site |
| 19 | GCACGGGCCGGGCGGCACCA | RASA2_1 target |
| | | site |
| 20 | TGCTGCGGCGGCTTCTTCCG | RASA2_2 target |
| | | site |
| 21 | GCGCTGGGCGCGAGGCTGAG | RASA2_3 target |
| | | site |
| 22 | CGAACTCCTTGGGCTAGAAG | ELOB_1 target site |
| 23 | TGCGGCCGCCATCCCGACGG | ELOB_2 target site |
| 24 | CTGGAAGCGGGCGGTATCGA | ELOB_3 target site |
| 25 | CGGAGGGTACCTCTGCACCG | GATA3_1 target |
| | | site |
| 26 | TCGACGAGGAGGCTCCACCC | GATA3_2 target |
| | | site |
| 27 | CAGGGCTGACTGTTACGACT | GATA3_3 target |
| | | site |
| 28 | TGTCCTGCGCCCGCGCGCCC | CISH_1 target site |
| 29 | GGCGGCTGGAGGGAACCAGT | CISH_2 target site |
| 30 | GTGGCGCGGACCGCCTGCGA | CISH_3 target site |
| 31 | AGAGGCAAGAGCAGCGACCG | PRDM1_1 target |
| | | site |
| 32 | GACGCGGGGAGAATGTGGAC | PRDM1_2 target |
| | | site |
| 33 | TTGCCTCTCCGCAACACTGG | PRDM1_3 target |
| | | site |
| 34 | ACGGAGGCTAAGCGTCGCAA | non-targeting_ |
| | | sp_1 |
| | | target site |
| 35 | UUGCACUGGAAGGGUAAAGC | CD5_1 gRNA |
| 36 | GGAGGCGACCAAGUAAAGGC | CD5_2 gRNA |
| 37 | GUCAGUGGGGGACCUCGCAG | CD5_3 gRNA |
| 38 | CGCGCGGGCAGCGUGAAGCG | KDM1A_1 gRNA |
| 39 | AGCGGCAGCAACCGGGACGG | KDM1A_2 gRNA |
| 40 | GCCCAGAAGCCCUAAGACCA | KDM1A_3 gRNA |
| 41 | UGUCGCUCCACAGGCGAGCA | VAV1_1 gRNA |
| 42 | CCUCUCAGGGCGACAGUUAC | VAV1_2 gRNA |
| 43 | GGCCAGCUAGACUAUGAGAU | VAV1_3 gRNA |
| 44 | GAACAGCUCGCUCCCGAAGA | CBLB_1 gRNA |
| 45 | CGCUGGGUUGCUCCUUCUUC | CBLB_2 gRNA |
| 46 | CGUCCAGGCAGACGGCGGUG | CBLB_3 gRNA |
| 47 | GGGACACGGGCGGGAUCGGU | DGKZ_1 gRNA |
| 48 | GGAGCGAGCGCGCGCCAUGG | DGKZ_2 gRNA |
| 49 | UCUUCGGGCACAGGUGAGCG | DGKZ_3 gRNA |
| 50 | GCCGAAUGGGAGCGGCGACC | MYB_1 gRNA |
| 51 | GGAUCCCUCGCCGACACCCG | MYB_2 gRNA |
| 52 | GAAACUUCGCCCCAGCGGUG | MYB_3 gRNA |
| 53 | GCACGGGCCGGGCGGCACCA | RASA2_1 gRNA |
| 54 | UGCUGCGGCGGCUUCUUCCG | RASA2_2 gRNA |
| 55 | GCGCUGGGCGCGAGGCUGAG | RASA2_3 gRNA |
| 56 | CGAACUCCUUGGGCUAGAAG | ELOB_1 gRNA |
| 57 | UGCGGCCGCCAUCCCGACGG | ELOB_2 gRNA |
| 58 | CUGGAAGCGGGCGGUAUCGA | ELOB_3 gRNA |
| 59 | CGGAGGGUACCUCUGCACCG | GATA3_1 gRNA |
| 60 | UCGACGAGGAGGCUCCACCC | GATA3_2 gRNA |
| 61 | CAGGGCUGACUGUUACGACU | GATA3_3 gRNA |
| 62 | UGUCCUGCGCCCGCGCGCCC | CISH_1 gRNA |
| 63 | GGCGGCUGGAGGGAACCAGU | CISH_2 gRNA |
| 64 | GUGGCGCGGACCGCCUGCGA | CISH_3 gRNA |
| 65 | AGAGGCAAGAGCAGCGACCG | PRDM1_1 gRNA |
| 66 | GACGCGGGGAGAAUGUGGAC | PRDM1_2 gRNA |
| 67 | UUGCCUCUCCGCAACACUGG | PRDM1_3 gRNA |
| 68 | ACGGAGGCUAAGCGUCGCAA | non-targeting_ |
| | | sp_1 |
| | | gRNA |
| 69 | GUUUAAGAGCUAUGCUGGAAACAGCAUAGCAAGUUUAAAU | SpCas9 gRNA |
| | AAGGCUAGUCCGUUAUCAACUUGAAAAAGUGGCACCGAGU | scaffold sequence |
| | CGGUGC | (RNA) |
| 70 | RTLVTFKDVFVDFTREEWKLLDTAQQILYRNVMLENYKNL | KRAB (AA) |
| | VSLGYQLTKPDVILRLEKGEEPWLV | |
| 71 | CGGACACTGGTGACCTTCAAGGATGTGTTTGTGGACTTCA | KRAB (nt) |
| | CCAGGGAGGAGTGGAAGCTGCTGGACACTGCTCAGCAGAT | |
| | CCTGTACAGAAATGTGATGCTGGAGAACTATAAGAACCTG | |
| | GTTTCCTTGGGTTATCAGCTTACTAAGCCAGATGTGATCC | |
| | TCCGGTTGGAGAAGGGAGAAGAGCCCTGGCTGGTG | |
| 72 | CCAAAGAAGAAGCGGAAGGTCGGTATCCACGGAGTCCCAG | dSpCas9 KRAB- |
| | CAGCCGACAAGAAGTACTCCATTGGGCTCGCCATCGGCAC | (nt) |
| | AAACAGCGTCGGCTGGGCCGTCATTACGGACGAGTACAAG | |
| | GTGCCGAGCAAAAAATTCAAAGTTCTGGGCAATACCGATC | |
| | GCCACAGCATAAAGAAGAACCTCATTGGCGCCCTCCTGTT | |
| | CGACTCCGGGGAAACCGCCGAAGCCACGCGGCTCAAAAGA | |
| | ACAGCACGGCGCAGATATACCCGCAGAAAGAATCGGATCT | |
| | GCTACCtgcaGGAGATCTTTAGTAATGAGATGGCTAAGGT | |
| | GGATGACTCTTTCTTCCATAGGCTGGAGGAGTCCTTTTTG | |
| | GTGGAGGAGGATAAAAAGCACGAGCGCCACCCAATCTTTG | |
| | GCAATATCGTGGACGAGGTGGCGTACCATGAAAAGTACCC | |
| | AACCATATATCATCTGAGGAAGAAGCTTGTAGACAGTACT | |
| | GATAAGGCTGACTTGCGGTTGATCTATCTCGCGCTGGCGC | |
| | ATATGATCAAATTTCGGGGACACTTCCTCATCGAGGGGGA | |
| | CCTGAACCCAGACAACAGCGATGTCGACAAACTCTTTATC | |
| | CAACTGGTTCAGACTTACAATCAGCTTTTCGAAGAGAACC | |
| | CGATCAACGCATCCGGAGTTGACGCCAAAGCAATCCTGAG | |
| | CGCTAGGCTGTCCAAATCCCGGCGGCTCGAAAACCTCATC | |
| | GCACAGCTCCCTGGGGAGAAGAAGAACGGCCTGTTTGGTA | |
| | ATCTTATCGCCCTGTCACTCGGGCTGACCCCCAACTTTAA | |
| | ATCTAACTTCGACCTGGCCGAAGATGCCAAGCTTCAACTG | |
| | AGCAAAGACACCTACGATGATGATCTCGACAATCTGCTGG | |
| | CCCAGATCGGCGACCAGTACGCAGACCTTTTTTTGGCGGC | |
| | AAAGAACCTGTCAGACGCCATTCTGCTGAGTGATATTCTG | |
| | CGAGTGAACACGGAGATCACCAAAGCTCCGCTGAGCGCTA | |
| | GTATGATCAAGCGCTATGATGAGCACCACCAAGACTTGAC | |
| | TTTGCTGAAGGCCCTTGTCAGACAGCAACTGCCTGAGAAG | |
| | TACAAGGAAATTTTCTTCGATCAGTCTAAAAATGGCTACG | |
| | CCGGATACATTGACGGCGGAGCAAGCCAGGAGGAATTTTA | |
| | CAAATTTATTAAGCCCATCTTGGAAAAAATGGACGGCACC | |
| | GAGGAGCTGCTGGTAAAGCTTAACAGAGAAGATCTGTTGC | |
| | GCAAACAGCGCACTTTCGACAATGGAAGCATCCCCCACCA | |
| | GATTCACCTGGGCGAACTGCACGCTATCCTCAGGCGGCAA | |
| | GAGGATTTCTACCCCTTTTTGAAAGATAACAGGGAAAAGA | |
| | TTGAGAAAATCCTCACATTTCGGATACCCTACTATGTAGG | |
| | CCCCCTCGCCCGGGGAAATTCCAGATTCGCGTGGATGACT | |
| | CGCAAATCAGAAGAGACCATCACTCCCTGGAACTTCGAGG | |
| | AAGTCGTGGATAAGGGGGCCTCTGCCCAGTCCTTCATCGA | |
| | AAGGATGACTAACTTTGATAAAAATCTGCCTAACGAAAAG | |
| | GTGCTTCCTAAACACTCTCTGCTGTACGAGTACTTCACAG | |
| | TTTATAACGAGCTCACCAAGGTCAAATACGTCACAGAAGG | |
| | GATGAGAAAGCCAGCATTCCTGTCTGGAGAGCAGAAGAAA | |
| | GCTATCGTGGACCTCCTCTTCAAGACGAACCGGAAAGTTA | |
| | CCGTGAAACAGCTCAAAGAAGACTATTTCAAAAAGATTGA | |
| | ATGTTTCGACTCTGTTGAAATCAGCGGAGTGGAGGATCGC | |
| | TTCAACGCATCCCTGGGAACGTATCACGATCTCCTGAAAA | |
| | TCATTAAAGACAAGGACTTCCTGGACAATGAGGAGAACGA | |
| | GGACATTCTTGAGGACATTGTCCTCACCCTTACGTTGTTT | |
| | GAAGATAGGGAGATGATTGAAGAACGCTTGAAAACTTACG | |
| | CTCATCTCTTCGACGACAAAGTCATGAAACAGCTCAAGAG | |
| | GCGCCGATATACAGGATGGGGGCGGCTGTCAAGAAAACTG | |
| | ATCAATGGgatcCGAGACAAGCAGAGTGGAAAGACAATCC | |
| | TGGATTTTCTTAAGTCCGATGGATTTGCCAACCGGAACTT | |
| | CATGCAGTTGATCCATGATGACTCTCTCACCTTTAAGGAG | |
| | GACATCCAGAAAGCACAAGTTTCTGGCCAGGGGGACAGTC | |
| | TTCACGAGCACATCGCTAATCTTGCAGGTAGCCCAGCTAT | |
| | CAAAAAGGGAATACTGCAGACCGTTAAGGTCGTGGATGAA | |
| | CTCGTCAAAGTAATGGGAAGGCATAAGCCCGAGAATATCG | |
| | TTATCGAGATGGCCCGAGAGAACCAAACTACCCAGAAGGG | |
| | ACAGAAGAACAGTAGGGAAAGGATGAAGAGGATTGAAGAG | |
| | GGTATAAAAGAACTGGGGTCCCAAATCCTTAAGGAACACC | |
| | CAGTTGAAAACACCCAGCTTCAGAATGAGAAGCTCTACCT | |
| | GTACTACCTGCAGAACGGCAGGGACATGTACGTGGATCAG | |
| | GAACTGGACATCAATCGGCTCTCCGACTACGACGTGGATG | |
| | CCATCGTGCCCCAGTCTTTTCTCAAAGATGATTCTATTGA | |
| | TAATAAAGTGTTGACAAGATCCGATAAAAATAGAGGGAAG | |
| | AGTGATAACGTCCCCTCAGAAGAAGTTGTCAAGAAAATGA | |
| | AAAATTATTGGCGGCAGCTGCTGAACGCCAAACTGATCAC | |
| | ACAACGGAAGTTCGATAATCTGACTAAGGCTGAACGAGGT | |
| | GGCCTGTCTGAGTTGGATAAAGCCGGCTTCATCAAAAGGC | |
| | AGCTTGTTGAGACACGCCAGATCACCAAgcacGTGGCCCA | |
| | AATTCTCGATTCACGCATGAACACCAAGTACGATGAAAAT | |
| | GACAAACTGATTCGAGAGGTGAAAGTTATTACTCTGAAGT | |
| | CTAAGCTGGTCTCAGATTTCAGAAAGGACTTTCAGTTTTA | |
| | TAAGGTGAGAGAGATCAACAATTACCACCATGCGCATGAT | |
| | GCCTACCTGAATGCAGTGGTAGGCACTGCACTTATCAAAA | |
| | AATATCCCAAGCTTGAATCTGAATTTGTTTACGGAGACTA | |
| | TAAAGTGTACGATGTTAGGAAAATGATCGCAAAGTCTGAG | |
| | CAGGAAATAGGCAAGGCCACCGCTAAGTACTTCTTTTACA | |
| | GCAATATTATGAATTTTTTCAAGACCGAGATTACACTGGC | |
| | CAATGGAGAGATTCGGAAGCGACCACTTATCGAAACAAAC | |
| | GGAGAAACAGGAGAAATCGTGTGGGACAAGGGTAGGGATT | |
| | TCGCGACAGTCCGGAAGGTCCTGTCCATGCCGCAGGTGAA | |
| | CATCGTTAAAAAGACCGAAGTACAGACCGGAGGCTTCTCC | |
| | AAGGAAAGTATCCTCCCGAAAAGGAACAGCGACAAGCTGA | |
| | TCGCACGCAAAAAAGATTGGGACCCCAAGAAATACGGCGG | |
| | ATTCGATTCTCCTACAGTCGCTTACAGTGTACTGGTTGTG | |
| | GCCAAAGTGGAGAAAGGGAAGTCTAAAAAACTCAAAAGCG | |
| | TCAAGGAACTGCTGGGCATCACAATCATGGAGCGATCAAG | |
| | CTTCGAAAAAAACCCCATCGACTTTCTCGAGGCGAAAGGA | |
| | TATAAAGAGGTCAAAAAAGACCTCATCATTAAGCTTCCCA | |
| | AGTACTCTCTCTTTGAGCTTGAAAACGGCCGGAAACGAAT | |
| | GCTCGCTAGTGCGGGCGAGCTGCAGAAAGGTAACGAGCTG | |
| | GCACTGCCCTCTAAATACGTTAATTTCTTGTATCTGGCCA | |
| | GCCACTATGAAAAGCTCAAAGGGTCTCCCGAAGATAATGA | |
| | GCAGAAGCAGCTGTTCGTGGAACAACACAAACACTACCTT | |
| | GATGAGATCATCGAGCAAATAAGCGAATTCTCCAAAAGAG | |
| | TGATCCTCGCCGACGCTAACCTCGATAAGGTGCTTTCTGC | |
| | TTACAATAAGCACAGGGATAAGCCCATCAGGGAGCAGGCA | |
| | GAAAACATTATCCACTTGTTTACTCTGACCAACTTGGGCG | |
| | CGCCTGCAGCCTTCAAGTACTTCGACACCACCATAGACAG | |
| | AAAGCGGTACACCTCTACAAAGGAGGTCCTGGACGCCACA | |
| | CTGATTCATCAGTCAATTACGGGGCTCTATGAAACAAGAA | |
| | TCGACCTCTCTCAGCTCGGTGGAGACAAAAGGCCGGCGGC | |
| | CACGAAAAAGGCCGGCCAGGCAAAAAAGAAAAAGGCTAGC | |
| | GATGCTAAGTCACTGACTGCCTGGTCCCGGACACTGGTGA | |
| | CCTTCAAGGATGTGTTTGTGGACTTCACCAGGGAGGAGTG | |
| | GAAGCTGCTGGACACTGCTCAGCAGATCCTGTACAGAAAT | |
| | GTGATGCTGGAGAACTATAAGAACCTGGTTTCCTTGGGTT | |
| | ATCAGCTTACTAAGCCAGATGTGATCCTCCGGTTGGAGAA | |
| | GGGAGAAGAGCCCTGGCTGGTGGAGAGAGAAATTCACCAA | |
| | GAGACCCATCCTGATTCAGAGACTGCATTTGAAATCAAAT | |
| | CATCAGTTCCGAAAAAGAAACGCAAAGTT | |
| 73 | PKKKRKVGIHGVPAADKKYSIGLAIGTNSVGWAVITDEYK | dSpCas9- KRAB |
| | VPSKKFKVLGNTDRHSIKKNLIGALLFDSGETAEATRLKR | (AA) |
| | TARRRYTRRKNRICYLQEIFSNEMAKVDDSFFHRLEESFL | |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDKRPAATKKAGQAKKKKAS | |
| | DAKSLTAWSRTLVTFKDVFVDFTREEWKLLDTAQQILYRN | |
| | VMLENYKNLVSLGYQLTKPDVILRLEKGEEPWLVEREIHQ | |
| | ETHPDSETAFEIKSSVPKKKRKV | |
| 74 | CACGTTAACCACGATCAGGAGTTTGACCCCCCTAAGGTGT | DNMT3A/L- |
| | ACCCACCCGTGCCAGCCGAGAAGAGGAAGCCCATCCGCGT | dCas9-KRAB (nt) |
| | GCTGTCCCTGTTCGACGGCATCGCCACAGGCCTGCTGGTG | |
| | CTGAAGGATCTGGGCATCCAGGTGGACAGATATATCGCCT | |
| | CCGAGGTGTGCGAGGATTCTATCACCGTGGGCATGGTGAG | |
| | GCACCAGGGCAAGATCATGTACGTGGGCGACGTGCGCAGC | |
| | GTGACACAGAAGCACATCCAGGAGTGGGGACCCTTCGACC | |
| | TGGTCATCGGAGGCAGCCCCTGTAATGACCTGTCCATCGT | |
| | GAACCCTGCAAGGAAGGGCCTGTATGAGGGAACCGGCAGA | |
| | CTGTTCTTTGAGTTCTACAGGCTGCTGCACGACGCCCGCC | |
| | CTAAGGAGGGCGATGACAGGCCATTCTTTTGGCTGTTTGA | |
| | GAACGTGGTGGCCATGGGCGTGAGCGACAAGCGGGATATC | |
| | TCCAGATTCCTGGAGTCTAATCCCGTGATGATCGATGCAA | |
| | AGGAGGTGTCTGCCGCACACAGGGCAAGGTACTTTTGGGG | |
| | AAATCTGCCTGGCATGAACCGCCCACTGGCCAGCACCGTG | |
| | AACGACAAGCTGGAGCTGCAGGAGTGCCTGGAGCACGGAA | |
| | GGATCGCCAAGTTCTCCAAGGTGCGGACAATCACCACAAG | |
| | ATCTAACAGCATCAAGCAGGGCAAGGATCAGCACTTCCCC | |
| | GTGTTCATGAATGAGAAGGAGGACATCCTGTGGTGTACCG | |
| | AGATGGAGCGCGTGTTCGGCTTTCCAGTGCACTATACAGA | |
| | CGTGAGCAATATGAGCCGGCTGGCAAGGCAGAGACTGCTG | |
| | GGCCGGTCCTGGTCTGTGCCAGTGATCAGACACCTGTTCG | |
| | CCCCCCTGAAGGAGTACTTTGCCTGCGTGTCTAGCGGCAA | |
| | CTCTAATGCCAACAGCAGAGGCCCTTCCTTTTCCTCTGGC | |
| | CTGGTGCCACTGTCTCTGAGGGGCAGCCACATGGGCCCCA | |
| | TGGAGATCTACAAGACCGTGTCCGCCTGGAAGAGGCAGCC | |
| | TGTGCGCGTGCTGTCTCTGTTCCGCAACATCGACAAGGTG | |
| | CTGAAGAGCCTGGGCTTTCTGGAGAGCGGATCCGGATCTG | |
| | GAGGAGGCACCCTGAAGTATGTGGAGGATGTGACAAATGT | |
| | GGTGCGGAGAGATGTGGAGAAGTGGGGCCCCTTCGATCTG | |
| | GTGTACGGATCCACCCAGCCACTGGGAAGCTCCTGCGATA | |
| | GGTGTCCAGGATGGTATATGTTCCAGTTTCACAGAATCCT | |
| | GCAGTACGCACTGCCAAGGCAGGAGAGCCAGCGCCCTTTC | |
| | TTTTGGATCTTTATGGACAACCTGCTGCTGACAGAGGATG | |
| | ACCAGGAGACAACAACCCGCTTCCTGCAGACAGAGGCAGT | |
| | GACCCTGCAGGATGTGAGGGGACGCGACTATCAGAATGCC | |
| | ATGCGGGTGTGGTCTAACATCCCTGGCCTGAAGAGCAAGC | |
| | ACGCCCCCCTGACCCCTAAGGAGGAGGAGTACCTGCAGGC | |
| | CCAGGTGCGGAGCAGATCCAAGCTGGATGCCCCTAAGGTG | |
| | GACCTGCTGGTGAAGAATTGTCTGCTGCCACTGCGGGAGT | |
| | ACTTCAAGTACTTTAGTCAGAATAGCCTGCCACTGGAGGC | |
| | AAGCGGATCCGGAAGGGCATCTCCTGGAATCCCAGGAAGC | |
| | ACCCGCAACCCCAAGAAGAAGCGGAAGGTGGGCATCCACG | |
| | GCGTGCCCGCCGCCGACAAGAAGTACAGCATCGGCCTGGC | |
| | CATCGGCACCAACAGCGTGGGCTGGGCCGTGATCACCGAC | |
| | GAGTACAAGGTGCCCAGCAAGAAGTTCAAGGTGCTGGGCA | |
| | ACACCGACCGGCACAGCATCAAGAAGAACCTGATCGGCGC | |
| | CCTGCTGTTCGACAGCGGCGAGACCGCCGAGGCCACCCGG | |
| | CTGAAGCGGACCGCCCGGCGGCGGTACACCCGGCGGAAGA | |
| | ACCGGATCTGCTACCTGCAGGAGATCTTCAGCAACGAGAT | |
| | GGCCAAGGTGGACGACAGCTTCTTCCACCGGCTGGAGGAG | |
| | AGCTTCCTGGTGGAGGAGGACAAGAAGCACGAGCGGCACC | |
| | CCATCTTCGGCAACATCGTGGACGAGGTGGCCTACCACGA | |
| | GAAGTACCCCACCATCTACCACCTGCGGAAGAAGCTGGTG | |
| | GACAGCACCGACAAGGCCGACCTGCGGCTGATCTACCTGG | |
| | CCCTGGCCCACATGATCAAGTTCCGGGGCCACTTCCTGAT | |
| | CGAGGGCGACCTGAACCCCGACAACAGCGACGTGGACAAG | |
| | CTGTTCATCCAGCTGGTGCAGACCTACAACCAGCTGTTCG | |
| | AGGAGAACCCCATCAACGCCAGCGGCGTGGACGCCAAGGC | |
| | CATCCTGAGCGCCCGGCTGAGCAAGAGCCGGCGGCTGGAG | |
| | AACCTGATCGCCCAGCTGCCCGGCGAGAAGAAGAACGGCC | |
| | TGTTCGGCAACCTGATCGCCCTGAGCCTGGGCCTGACCCC | |
| | CAACTTCAAGAGCAACTTCGACCTGGCCGAGGACGCCAAG | |
| | CTGCAGCTGAGCAAGGACACCTACGACGACGACCTGGACA | |
| | ACCTGCTGGCCCAGATCGGCGACCAGTACGCCGACCTGTT | |
| | CCTGGCCGCCAAGAACCTGAGCGACGCCATCCTGCTGAGC | |
| | GACATCCTGCGGGTGAACACCGAGATCACCAAGGCCCCCC | |
| | TGAGCGCCAGCATGATCAAGCGGTACGACGAGCACCACCA | |
| | GGACCTGACCCTGCTGAAGGCCCTGGTGCGGCAGCAGCTG | |
| | CCCGAGAAGTACAAGGAGATCTTCTTCGACCAGAGCAAGA | |
| | ACGGCTACGCCGGCTACATCGACGGCGGCGCCAGCCAGGA | |
| | GGAGTTCTACAAGTTCATCAAGCCCATCCTGGAGAAGATG | |
| | GACGGCACCGAGGAGCTGCTGGTGAAGCTGAACCGGGAGG | |
| | ACCTGCTGCGGAAGCAGCGGACCTTCGACAACGGCAGCAT | |
| | CCCCCACCAGATCCACCTGGGCGAGCTGCACGCCATCCTG | |
| | CGGCGGCAGGAGGACTTCTACCCCTTCCTGAAGGACAACC | |
| | GGGAGAAGATCGAGAAGATCCTGACCTTCCGGATCCCCTA | |
| | CTACGTGGGCCCCCTGGCCCGGGGCAACAGCCGGTTCGCC | |
| | TGGATGACCCGGAAGAGCGAGGAGACCATCACCCCCTGGA | |
| | ACTTCGAGGAGGTGGTGGACAAGGGCGCCAGCGCCCAGAG | |
| | CTTCATCGAGCGGATGACCAACTTCGACAAGAACCTGCCC | |
| | AACGAGAAGGTGCTGCCCAAGCACAGCCTGCTGTACGAGT | |
| | ACTTCACCGTGTACAACGAGCTGACCAAGGTGAAGTACGT | |
| | GACCGAGGGCATGCGGAAGCCCGCCTTCCTGAGCGGCGAG | |
| | CAGAAGAAGGCCATCGTGGACCTGCTGTTCAAGACCAACC | |
| | GGAAGGTGACCGTGAAGCAGCTGAAGGAGGACTACTTCAA | |
| | GAAGATCGAGTGCTTCGACAGCGTGGAGATCAGCGGCGTG | |
| | GAGGACCGGTTCAACGCCAGCCTGGGCACCTACCACGACC | |
| | TGCTGAAGATCATCAAGGACAAGGACTTCCTGGACAACGA | |
| | GGAGAACGAGGACATCCTGGAGGACATCGTGCTGACCCTG | |
| | ACCCTGTTCGAGGACCGGGAGATGATCGAGGAGCGGCTGA | |
| | AGACCTACGCCCACCTGTTCGACGACAAGGTGATGAAGCA | |
| | GCTGAAGCGGCGGCGGTACACCGGCTGGGGCCGGCTGAGC | |
| | CGGAAGCTGATCAACGGCATCCGGGACAAGCAGAGCGGCA | |
| | AGACCATCCTGGACTTCCTGAAGAGCGACGGCTTCGCCAA | |
| | CCGGAACTTCATGCAGCTGATCCACGACGACAGCCTGACC | |
| | TTCAAGGAGGACATCCAGAAGGCCCAGGTGAGCGGCCAGG | |
| | GCGACAGCCTGCACGAGCACATCGCCAACCTGGCCGGCAG | |
| | CCCCGCCATCAAGAAGGGCATCCTGCAGACCGTGAAGGTG | |
| | GTGGACGAGCTGGTGAAGGTGATGGGCCGGCACAAGCCCG | |
| | AGAACATCGTGATCGAGATGGCCCGGGAGAACCAGACCAC | |
| | CCAGAAGGGCCAGAAGAACAGCCGGGAGCGGATGAAGCGG | |
| | ATCGAGGAGGGCATCAAGGAGCTGGGCAGCCAGATCCTGA | |
| | AGGAGCACCCCGTGGAGAACACCCAGCTGCAGAACGAGAA | |
| | GCTGTACCTGTACTACCTGCAGAACGGCCGGGACATGTAC | |
| | GTGGACCAGGAGCTGGACATCAACCGGCTGAGCGACTACG | |
| | ACGTGGACGCCATCGTGCCCCAGAGCTTCCTGAAGGACGA | |
| | CAGCATCGACAACAAGGTGCTGACCCGGAGCGACAAGAAC | |
| | CGGGGCAAGAGCGACAACGTGCCCAGCGAGGAGGTGGTGA | |
| | AGAAGATGAAGAACTACTGGCGGCAGCTGCTGAACGCCAA | |
| | GCTGATCACCCAGCGGAAGTTCGACAACCTGACCAAGGCC | |
| | GAGCGGGGCGGCCTGAGCGAGCTGGACAAGGCCGGCTTCA | |
| | TCAAGCGGCAGCTGGTGGAGACCCGGCAGATCACCAAGCA | |
| | CGTGGCCCAGATCCTGGACAGCCGGATGAACACCAAGTAC | |
| | GACGAGAACGACAAGCTGATCCGGGAGGTGAAGGTGATCA | |
| | CCCTGAAGAGCAAGCTGGTGAGCGACTTCCGGAAGGACTT | |
| | CCAGTTCTACAAGGTGCGGGAGATCAACAACTACCACCAC | |
| | GCCCACGACGCCTACCTGAACGCCGTGGTGGGCACCGCCC | |
| | TGATCAAGAAGTACCCCAAGCTGGAGAGCGAGTTCGTGTA | |
| | CGGCGACTACAAGGTGTACGACGTGCGGAAGATGATCGCC | |
| | AAGAGCGAGCAGGAGATCGGCAAGGCCACCGCCAAGTACT | |
| | TCTTCTACAGCAACATCATGAACTTCTTCAAGACCGAGAT | |
| | CACCCTGGCCAACGGCGAGATCCGGAAGCGGCCCCTGATC | |
| | GAGACCAACGGCGAGACCGGCGAGATCGTGTGGGACAAGG | |
| | GCCGGGACTTCGCCACCGTGCGGAAGGTGCTGAGCATGCC | |
| | CCAGGTGAACATCGTGAAGAAGACCGAGGTGCAGACCGGC | |
| | GGCTTCAGCAAGGAGAGCATCCTGCCCAAGCGGAACAGCG | |
| | ACAAGCTGATCGCCCGGAAGAAGGACTGGGACCCCAAGAA | |
| | GTACGGCGGCTTCGACAGCCCCACCGTGGCCTACAGCGTG | |
| | CTGGTGGTGGCCAAGGTGGAGAAGGGCAAGAGCAAGAAGC | |
| | TGAAGAGCGTGAAGGAGCTGCTGGGCATCACCATCATGGA | |
| | GCGGAGCAGCTTCGAGAAGAACCCCATCGACTTCCTGGAG | |
| | GCCAAGGGCTACAAGGAGGTGAAGAAGGACCTGATCATCA | |
| | AGCTGCCCAAGTACAGCCTGTTCGAGCTGGAGAACGGCCG | |
| | GAAGCGGATGCTGGCCAGCGCCGGCGAGCTGCAGAAGGGC | |
| | AACGAGCTGGCCCTGCCCAGCAAGTACGTGAACTTCCTGT | |
| | ACCTGGCCAGCCACTACGAGAAGCTGAAGGGCAGCCCCGA | |
| | GGACAACGAGCAGAAGCAGCTGTTCGTGGAGCAGCACAAG | |
| | CACTACCTGGACGAGATCATCGAGCAGATCAGCGAGTTCA | |
| | GCAAGCGGGTGATCCTGGCCGACGCCAACCTGGACAAGGT | |
| | GCTGAGCGCCTACAACAAGCACCGGGACAAGCCCATCCGG | |
| | GAGCAGGCCGAGAACATCATCCACCTGTTCACCCTGACCA | |
| | ACCTGGGCGCCCCCGCCGCCTTCAAGTACTTCGACACCAC | |
| | CATCGACCGGAAGCGGTACACCAGCACCAAGGAGGTGCTG | |
| | GACGCCACCCTGATCCACCAGAGCATCACCGGCCTGTACG | |
| | AGACCCGGATCGACCTGAGCCAGCTGGGCGGCGACAGCGG | |
| | CGGCAAGCGGCCCGCCGCCACCAAGAAGGCCGGCCAGGCC | |
| | AAGAAGAAGAAGGCTAGCGATGCTAAGTCACTGACTGCCT | |
| | GGTCCCGGACACTGGTGACCTTCAAGGATGTGTTTGTGGA | |
| | CTTCACCAGGGAGGAGTGGAAGCTGCTGGACACTGCTCAG | |
| | CAGATCCTGTACAGAAATGTGATGCTGGAGAACTATAAGA | |
| | ACCTGGTTTCCTTGGGTTATCAGCTTACTAAGCCAGATGT | |
| | GATCCTCCGGTTGGAGAAGGGAGAAGAGCCCTGGCTGGTG | |
| | GAGAGAGAAATTCACCAAGAGACCCATCCTGATTCAGAGA | |
| | CTGCATTTGAAATCAAATCATCAGTTCCGAAAAAGAAACG | |
| | CAAAGTT | |
| 75 | HVNHDQEFDPPKVYPPVPAEKRKPIRVLSLFDGIATGLLV | DNMT3A/L- |
| | LKDLGIQVDRYIASEVCEDSITVGMVRHQGKIMYVGDVRS | dCas9-KRAB(AA) |
| | VTQKHIQEWGPFDLVIGGSPCNDLSIVNPARKGLYEGTGR | |
| | LFFEFYRLLHDARPKEGDDRPFFWLFENVVAMGVSDKRDI | |
| | SRFLESNPVMIDAKEVSAAHRARYFWGNLPGMNRPLASTV | |
| | NDKLELQECLEHGRIAKFSKVRTITTRSNSIKQGKDQHFP | |
| | VFMNEKEDILWCTEMERVFGFPVHYTDVSNMSRLARQRLL | |
| | GRSWSVPVIRHLFAPLKEYFACVSSGNSNANSRGPSFSSG | |
| | LVPLSLRGSHMGPMEIYKTVSAWKRQPVRVLSLFRNIDKV | |
| | LKSLGFLESGSGSGGGTLKYVEDVTNVVRRDVEKWGPFDL | |
| | VYGSTQPLGSSCDRCPGWYMFQFHRILQYALPRQESQRPF | |
| | FWIFMDNLLLTEDDQETTTRFLQTEAVTLQDVRGRDYQNA | |
| | MRVWSNIPGLKSKHAPLTPKEEEYLQAQVRSRSKLDAPKV | |
| | DLLVKNCLLPLREYFKYFSQNSLPLEASGSGRASPGIPGS | |
| | TRNPKKKRKVGIHGVPAADKKYSIGLAIGTNSVGWAVITD | |
| | EYKVPSKKFKVLGNTDRHSIKKNLIGALLFDSGETAEATR | |
| | LKRTARRRYTRRKNRICYLQEIFSNEMAKVDDSFFHRLEE | |
| | SFLVEEDKKHERHPIFGNIVDEVAYHEKYPTIYHLRKKLV | |
| | DSTDKADLRLIYLALAHMIKFRGHFLIEGDLNPDNSDVDK | |
| | LFIQLVQTYNQLFEENPINASGVDAKAILSARLSKSRRLE | |
| | NLIAQLPGEKKNGLFGNLIALSLGLTPNFKSNFDLAEDAK | |
| | LQLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEKM | |
| | DGTEELLVKLNREDLLRKQRTFDNGSIPHQIHLGELHAIL | |
| | RRQEDFYPFLKDNREKIEKILTFRIPYYVGPLARGNSRFA | |
| | WMTRKSEETITPWNFEEVVDKGASAQSFIERMTNFDKNLP | |
| | NEKVLPKHSLLYEYFTVYNELTKVKYVTEGMRKPAFLSGE | |
| | QKKAIVDLLFKTNRKVTVKQLKEDYFKKIECFDSVEISGV | |
| | EDRFNASLGTYHDLLKIIKDKDFLDNEENEDILEDIVLTL | |
| | TLFEDREMIEERLKTYAHLFDDKVMKQLKRRRYTGWGRLS | |
| | RKLINGIRDKQSGKTILDFLKSDGFANRNFMQLIHDDSLT | |
| | FKEDIQKAQVSGQGDSLHEHIANLAGSPAIKKGILQTVKV | |
| | VDELVKVMGRHKPENIVIEMARENQTTQKGQKNSRERMKR | |
| | IEEGIKELGSQILKEHPVENTQLQNEKLYLYYLQNGRDMY | |
| | VDQELDINRLSDYDVDAIVPQSFLKDDSIDNKVLTRSDKN | |
| | RGKSDNVPSEEVVKKMKNYWRQLLNAKLITQRKFDNLTKA | |
| | ERGGLSELDKAGFIKRQLVETRQITKHVAQILDSRMNTKY | |
| | DENDKLIREVKVITLKSKLVSDFRKDFQFYKVREINNYHH | |
| | AHDAYLNAVVGTALIKKYPKLESEFVYGDYKVYDVRKMIA | |
| | KSEQEIGKATAKYFFYSNIMNFFKTEITLANGEIRKRPLI | |
| | ETNGETGEIVWDKGRDFATVRKVLSMPQVNIVKKTEVQTG | |
| | GFSKESILPKRNSDKLIARKKDWDPKKYGGFDSPTVAYSV | |
| | LVVAKVEKGKSKKLKSVKELLGITIMERSSFEKNPIDFLE | |
| | AKGYKEVKKDLIIKLPKYSLFELENGRKRMLASAGELQKG | |
| | NELALPSKYVNFLYLASHYEKLKGSPEDNEQKQLFVEQHK | |
| | HYLDEIIEQISEFSKRVILADANLDKVLSAYNKHRDKPIR | |
| | EQAENIIHLFTLTNLGAPAAFKYFDTTIDRKRYTSTKEVL | |
| | DATLIHQSITGLYETRIDLSQLGGDSGGKRPAATKKAGQA | |
| | KKKKASDAKSLTAWSRTLVTFKDVFVDFTREEWKLLDTAQ | |
| | QILYRNVMLENYKNLVSLGYQLTKPDVILRLEKGEEPWLV | |
| | EREIHQETHPDSETAFEIKSSVPKKKRKV | |
| 76 | gacgcattggacgattttgatctggatatgctgggaagtg | VP64-dSpCas9- |
| | acgccctcgatgattttgaccttgacatgcttggttcgga | NLS-VP64 (nt) |
| | tgcccttgatgactttgacctcgacatgctcggcagtgac | |
| | gcccttgatgatttcgacctggacatgGTTAACCCAAAGA | |
| | AGAAGCGGAAGGTCGGTATCCACGGAGTCCCAGCAGCCGA | |
| | CAAGAAGTACTCCATTGGGCTCGCCATCGGCACAAACAGC | |
| | GTCGGCTGGGCCGTCATTACGGACGAGTACAAGGTGCCGA | |
| | GCAAAAAATTCAAAGTTCTGGGCAATACCGATCGCCACAG | |
| | CATAAAGAAGAACCTCATTGGCGCCCTCCTGTTCGACTCC | |
| | GGGGAAACCGCCGAAGCCACGCGGCTCAAAAGAACAGCAC | |
| | GGCGCAGATATACCCGCAGAAAGAATCGGATCTGCTACCt | |
| | gcaGGAGATCTTTAGTAATGAGATGGCTAAGGTGGATGAC | |
| | TCTTTCTTCCATAGGCTGGAGGAGTCCTTTTTGGTGGAGG | |
| | AGGATAAAAAGCACGAGCGCCACCCAATCTTTGGCAATAT | |
| | CGTGGACGAGGTGGCGTACCATGAAAAGTACCCAACCATA | |
| | TATCATCTGAGGAAGAAGCTTGTAGACAGTACTGATAAGG | |
| | CTGACTTGCGGTTGATCTATCTCGCGCTGGCGCATATGAT | |
| | CAAATTTCGGGGACACTTCCTCATCGAGGGGGACCTGAAC | |
| | CCAGACAACAGCGATGTCGACAAACTCTTTATCCAACTGG | |
| | TTCAGACTTACAATCAGCTTTTCGAAGAGAACCCGATCAA | |
| | CGCATCCGGAGTTGACGCCAAAGCAATCCTGAGCGCTAGG | |
| | CTGTCCAAATCCCGGCGGCTCGAAAACCTCATCGCACAGC | |
| | TCCCTGGGGAGAAGAAGAACGGCCTGTTTGGTAATCTTAT | |
| | CGCCCTGTCACTCGGGCTGACCCCCAACTTTAAATCTAAC | |
| | TTCGACCTGGCCGAAGATGCCAAGCTTCAACTGAGCAAAG | |
| | ACACCTACGATGATGATCTCGACAATCTGCTGGCCCAGAT | |
| | CGGCGACCAGTACGCAGACCTTTTTTTGGCGGCAAAGAAC | |
| | CTGTCAGACGCCATTCTGCTGAGTGATATTCTGCGAGTGA | |
| | ACACGGAGATCACCAAAGCTCCGCTGAGCGCTAGTATGAT | |
| | CAAGCGCTATGATGAGCACCACCAAGACTTGACTTTGCTG | |
| | AAGGCCCTTGTCAGACAGCAACTGCCTGAGAAGTACAAGG | |
| | AAATTTTCTTCGATCAGTCTAAAAATGGCTACGCCGGATA | |
| | CATTGACGGCGGAGCAAGCCAGGAGGAATTTTACAAATTT | |
| | ATTAAGCCCATCTTGGAAAAAATGGACGGCACCGAGGAGC | |
| | TGCTGGTAAAGCTTAACAGAGAAGATCTGTTGCGCAAACA | |
| | GCGCACTTTCGACAATGGAAGCATCCCCCACCAGATTCAC | |
| | CTGGGCGAACTGCACGCTATCCTCAGGCGGCAAGAGGATT | |
| | TCTACCCCTTTTTGAAAGATAACAGGGAAAAGATTGAGAA | |
| | AATCCTCACATTTCGGATACCCTACTATGTAGGCCCCCTC | |
| | GCCCGGGGAAATTCCAGATTCGCGTGGATGACTCGCAAAT | |
| | CAGAAGAGACCATCACTCCCTGGAACTTCGAGGAAGTCGT | |
| | GGATAAGGGGGCCTCTGCCCAGTCCTTCATCGAAAGGATG | |
| | ACTAACTTTGATAAAAATCTGCCTAACGAAAAGGTGCTTC | |
| | CTAAACACTCTCTGCTGTACGAGTACTTCACAGTTTATAA | |
| | CGAGCTCACCAAGGTCAAATACGTCACAGAAGGGATGAGA | |
| | AAGCCAGCATTCCTGTCTGGAGAGCAGAAGAAAGCTATCG | |
| | TGGACCTCCTCTTCAAGACGAACCGGAAAGTTACCGTGAA | |
| | ACAGCTCAAAGAAGACTATTTCAAAAAGATTGAATGTTTC | |
| | GACTCTGTTGAAATCAGCGGAGTGGAGGATCGCTTCAACG | |
| | CATCCCTGGGAACGTATCACGATCTCCTGAAAATCATTAA | |
| | AGACAAGGACTTCCTGGACAATGAGGAGAACGAGGACATT | |
| | CTTGAGGACATTGTCCTCACCCTTACGTTGTTTGAAGATA | |
| | GGGAGATGATTGAAGAACGCTTGAAAACTTACGCTCATCT | |
| | CTTCGACGACAAAGTCATGAAACAGCTCAAGAGGCGCCGA | |
| | TATACAGGATGGGGGCGGCTGTCAAGAAAACTGATCAATG | |
| | GgatcCGAGACAAGCAGAGTGGAAAGACAATCCTGGATTT | |
| | TCTTAAGTCCGATGGATTTGCCAACCGGAACTTCATGCAG | |
| | TTGATCCATGATGACTCTCTCACCTTTAAGGAGGACATCC | |
| | AGAAAGCACAAGTTTCTGGCCAGGGGGACAGTCTTCACGA | |
| | GCACATCGCTAATCTTGCAGGTAGCCCAGCTATCAAAAAG | |
| | GGAATACTGCAGACCGTTAAGGTCGTGGATGAACTCGTCA | |
| | AAGTAATGGGAAGGCATAAGCCCGAGAATATCGTTATCGA | |
| | GATGGCCCGAGAGAACCAAACTACCCAGAAGGGACAGAAG | |
| | AACAGTAGGGAAAGGATGAAGAGGATTGAAGAGGGTATAA | |
| | AAGAACTGGGGTCCCAAATCCTTAAGGAACACCCAGTTGA | |
| | AAACACCCAGCTTCAGAATGAGAAGCTCTACCTGTACTAC | |
| | CTGCAGAACGGCAGGGACATGTACGTGGATCAGGAACTGG | |
| | ACATCAATCGGCTCTCCGACTACGACGTGGATGCCATCGT | |
| | GCCCCAGTCTTTTCTCAAAGATGATTCTATTGATAATAAA | |
| | GTGTTGACAAGATCCGATAAAAATAGAGGGAAGAGTGATA | |
| | ACGTCCCCTCAGAAGAAGTTGTCAAGAAAATGAAAAATTA | |
| | TTGGCGGCAGCTGCTGAACGCCAAACTGATCACACAACGG | |
| | AAGTTCGATAATCTGACTAAGGCTGAACGAGGTGGCCTGT | |
| | CTGAGTTGGATAAAGCCGGCTTCATCAAAAGGCAGCTTGT | |
| | TGAGACACGCCAGATCACCAAgcacGTGGCCCAAATTCTC | |
| | GATTCACGCATGAACACCAAGTACGATGAAAATGACAAAC | |
| | TGATTCGAGAGGTGAAAGTTATTACTCTGAAGTCTAAGCT | |
| | GGTCTCAGATTTCAGAAAGGACTTTCAGTTTTATAAGGTG | |
| | AGAGAGATCAACAATTACCACCATGCGCATGATGCCTACC | |
| | TGAATGCAGTGGTAGGCACTGCACTTATCAAAAAATATCC | |
| | CAAGCTTGAATCTGAATTTGTTTACGGAGACTATAAAGTG | |
| | TACGATGTTAGGAAAATGATCGCAAAGTCTGAGCAGGAAA | |
| | TAGGCAAGGCCACCGCTAAGTACTTCTTTTACAGCAATAT | |
| | TATGAATTTTTTCAAGACCGAGATTACACTGGCCAATGGA | |
| | GAGATTCGGAAGCGACCACTTATCGAAACAAACGGAGAAA | |
| | CAGGAGAAATCGTGTGGGACAAGGGTAGGGATTTCGCGAC | |
| | AGTCCGGAAGGTCCTGTCCATGCCGCAGGTGAACATCGTT | |
| | AAAAAGACCGAAGTACAGACCGGAGGCTTCTCCAAGGAAA | |
| | GTATCCTCCCGAAAAGGAACAGCGACAAGCTGATCGCACG | |
| | CAAAAAAGATTGGGACCCCAAGAAATACGGCGGATTCGAT | |
| | TCTCCTACAGTCGCTTACAGTGTACTGGTTGTGGCCAAAG | |
| | TGGAGAAAGGGAAGTCTAAAAAACTCAAAAGCGTCAAGGA | |
| | ACTGCTGGGCATCACAATCATGGAGCGATCAAGCTTCGAA | |
| | AAAAACCCCATCGACTTTCTCGAGGCGAAAGGATATAAAG | |
| | AGGTCAAAAAAGACCTCATCATTAAGCTTCCCAAGTACTC | |
| | TCTCTTTGAGCTTGAAAACGGCCGGAAACGAATGCTCGCT | |
| | AGTGCGGGCGAGCTGCAGAAAGGTAACGAGCTGGCACTGC | |
| | CCTCTAAATACGTTAATTTCTTGTATCTGGCCAGCCACTA | |
| | TGAAAAGCTCAAAGGGTCTCCCGAAGATAATGAGCAGAAG | |
| | CAGCTGTTCGTGGAACAACACAAACACTACCTTGATGAGA | |
| | TCATCGAGCAAATAAGCGAATTCTCCAAAAGAGTGATCCT | |
| | CGCCGACGCTAACCTCGATAAGGTGCTTTCTGCTTACAAT | |
| | AAGCACAGGGATAAGCCCATCAGGGAGCAGGCAGAAAACA | |
| | TTATCCACTTGTTTACTCTGACCAACTTGGGCGCGCCTGC | |
| | AGCCTTCAAGTACTTCGACACCACCATAGACAGAAAGCGG | |
| | TACACCTCTACAAAGGAGGTCCTGGACGCCACACTGATTC | |
| | ATCAGTCAATTACGGGGCTCTATGAAACAAGAATCGACCT | |
| | CTCTCAGCTCGGTGGAGACAAAAGGCCGGCGGCCACGAAA | |
| | AAGGCCGGCCAGGCAAAAAAGAAAAAGGctagCcgcgccg | |
| | acgcgctggacgatttcgatctcgacatgctgggttctga | |
| | tgccctcgatgactttgacctggatatgttgggaagcgac | |
| | gcattggatgactttgatctggacatgctcggctccgatg | |
| | ctctggacgatttcgatctcgatatgtta | |
| 77 | DALDDFDLDMLGSDALDDFDLDMLGSDALDDFDLDMLGSD | VP64-dSpCas9- |
| | ALDDFDLDMVNPKKKRKVGIHGVPAADKKYSIGLAIGTNS | NLS-VP64 (AA) |
| | VGWAVITDEYKVPSKKFKVLGNTDRHSIKKNLIGALLFDS | |
| | GETAEATRLKRTARRRYTRRKNRICYLQEIFSNEMAKVDD | |
| | SFFHRLEESFLVEEDKKHERHPIFGNIVDEVAYHEKYPTI | |
| | YHLRKKLVDSTDKADLRLIYLALAHMIKFRGHFLIEGDLN | |
| | PDNSDVDKLFIQLVQTYNQLFEENPINASGVDAKAILSAR | |
| | LSKSRRLENLIAQLPGEKKNGLFGNLIALSLGLTPNFKSN | |
| | FDLAEDAKLQLSKDTYDDDLDNLLAQIGDQYADLFLAAKN | |
| | LSDAILLSDILRVNTEITKAPLSASMIKRYDEHHQDLTLL | |
| | KALVRQQLPEKYKEIFFDQSKNGYAGYIDGGASQEEFYKF | |
| | IKPILEKMDGTEELLVKLNREDLLRKQRTFDNGSIPHQIH | |
| | LGELHAILRRQEDFYPFLKDNREKIEKILTFRIPYYVGPL | |
| | ARGNSRFAWMTRKSEETITPWNFEEVVDKGASAQSFIERM | |
| | TNFDKNLPNEKVLPKHSLLYEYFTVYNELTKVKYVTEGMR | |
| | KPAFLSGEQKKAIVDLLFKTNRKVTVKQLKEDYFKKIECF | |
| | DSVEISGVEDRFNASLGTYHDLLKIIKDKDFLDNEENEDI | |
| | LEDIVLTLTLFEDREMIEERLKTYAHLFDDKVMKQLKRRR | |
| | YTGWGRLSRKLINGIRDKQSGKTILDFLKSDGFANRNFMQ | |
| | LIHDDSLTFKEDIQKAQVSGQGDSLHEHIANLAGSPAIKK | |
| | GILQTVKVVDELVKVMGRHKPENIVIEMARENQTTQKGQK | |
| | NSRERMKRIEEGIKELGSQILKEHPVENTQLQNEKLYLYY | |
| | LQNGRDMYVDQELDINRLSDYDVDAIVPQSFLKDDSIDNK | |
| | VLTRSDKNRGKSDNVPSEEVVKKMKNYWRQLLNAKLITQR | |
| | KFDNLTKAERGGLSELDKAGFIKRQLVETRQITKHVAQIL | |
| | DSRMNTKYDENDKLIREVKVITLKSKLVSDFRKDFQFYKV | |
| | REINNYHHAHDAYLNAVVGTALIKKYPKLESEFVYGDYKV | |
| | YDVRKMIAKSEQEIGKATAKYFFYSNIMNFFKTEITLANG | |
| | EIRKRPLIETNGETGEIVWDKGRDFATVRKVLSMPQVNIV | |
| | KKTEVQTGGFSKESILPKRNSDKLIARKKDWDPKKYGGFD | |
| | SPTVAYSVLVVAKVEKGKSKKLKSVKELLGITIMERSSFE | |
| | KNPIDFLEAKGYKEVKKDLIIKLPKYSLFELENGRKRMLA | |
| | SAGELQKGNELALPSKYVNFLYLASHYEKLKGSPEDNEQK | |
| | QLFVEQHKHYLDEIIEQISEFSKRVILADANLDKVLSAYN | |
| | KHRDKPIREQAENIIHLFTLTNLGAPAAFKYFDTTIDRKR | |
| | YTSTKEVLDATLIHQSITGLYETRIDLSQLGGDKRPAATK | |
| | KAGQAKKKKASRADALDDFDLDMLGSDALDDFDLDMLGSD | |
| | ALDDFDLDMLGSDALDDFDLDML | |
| 78 | GAGAGCTATCACCTAAGTGT | IL-2_1 target site |
| 79 | GAGAGCUAUCACCUAAGUGU | IL-2_1 gRNA |
| 80 | ACCATTGCCGGAAACTACCG | Med12_1 target |
| | | site |
| 81 | GTGCCCCGGGAGTTTTTCGG | Med12_2 target |
| | | site |
| 82 | ACGGCGGCCGAGAGACAACA | Med12_3 target |
| | | site |
| 83 | CGAGGTACGCCGGGAACCAT | Med12_4 target |
| | | site |
| 84 | CGCCACCGCCGAAAAACTCC | Med12_5 target |
| | | site |
| 85 | CGATGGTTCCCGGCGTACCT | Med12_6 target |
| | | site |
| 86 | CGGCGGCCGAGAGACAACAA | Med12_7 target |
| | | site |
| 87 | TCCTGAGGGTAAACATCGGG | Med12_8 target |
| | | site |
| 88 | TTCGTAGCTCAAGATCCCGA | Med12_9 target |
| | | site |
| 89 | GCTGACTGGGGGAACGGGAA | Med12_10 target |
| | | site |
| 90 | GGCTGGTGCCTCCGGCGCTA | Med12_11 target |
| | | site |
| 91 | ACCAUUGCCGGAAACUACCG | Med12_1 gRNA |
| 92 | GUGCCCCGGGAGUUUUUCGG | Med12_2 gRNA |
| 93 | ACGGCGGCCGAGAGACAACA | Med12_3 gRNA |
| 94 | CGAGGUACGCCGGGAACCAU | Med12_4 gRNA |
| 95 | CGCCACCGCCGAAAAACUCC | Med12_5 gRNA |
| 96 | CGAUGGUUCCCGGCGUACCU | Med12_6 gRNA |
| 97 | CGGCGGCCGAGAGACAACAA | Med12_7 gRNA |
| 98 | UCCUGAGGGUAAACAUCGGG | Med12_8 gRNA |
| 99 | UUCGUAGCUCAAGAUCCCGA | Med12_9 gRNA |
| 100 | GCUGACUGGGGGAACGGGAA | Med12_10 gRNA |
| 101 | GGCUGGUGCCUCCGGCGCUA | Med12_11 gRNA |
| 102 | AAAGTTCCGGCCCGCGGTAG | CCNC_1 target site |
| 103 | GGGCCGGAACTTTTGTCGAT | CCNC_2 target site |
| 104 | CGACGGCGAAAGGAAGAGGA | CCNC_3 target site |
| 105 | CGAGGAGCGCGGTTACCGGA | CCNC_4 target site |
| 106 | CGGCCGGCGTGAAGGAGACT | CCNC_5 target site |
| 107 | TCACGAGAGCTCGCGGCGGT | CCNC_6 target site |
| 108 | CTGGGTCTATGGTCGCTCCG | CCNC_7 target site |
| 109 | GAACTTTTGTCGATAGGAAC | CCNC_8 target site |
| 110 | GCTGATTTGATCGAGGAGCG | CCNC_9 target site |
| 111 | GGAGGAGCGCGGTTACCGGA | CCNC_10 target |
| | | site |
| 112 | GTGGGTCTATGGTCGCTCCG | CCNC_11 target |
| | | site |
| 113 | AAAGUUCCGGCCCGCGGUAG | CCNC_1 gRNA |
| 114 | GGGCCGGAACUUUUGUCGAU | CCNC_2 gRNA |
| 115 | CGACGGCGAAAGGAAGAGGA | CCNC_3 gRNA |
| 116 | CGAGGAGCGCGGUUACCGGA | CCNC_4 gRNA |
| 117 | CGGCCGGCGUGAAGGAGACU | CCNC_5 gRNA |
| 118 | UCACGAGAGCUCGCGGCGGU | CCNC_6 gRNA |
| 119 | CUGGGUCUAUGGUCGCUCCG | CCNC_7 gRNA |
| 120 | GAACUUUUGUCGAUAGGAAC | CCNC_8 gRNA |
| 121 | GCUGAUUUGAUCGAGGAGCG | CCNC_9 gRNA |
| 122 | GGAGGAGCGCGGUUACCGGA | CCNC_10 gRNA |
| 123 | GUGGGUCUAUGGUCGCUCCG | CCNC_11 gRNA |
| 124 | MKRNYILGLDIGITSVGYGIIDYETRDVIDAGVRLFKEAN | SaCas9 (AA) |
| | VENNEGRRSKRGARRLKRRRRHRIQRVKKLLFDYNLLTDH | |
| | SELSGINPYEARVKGLSQKLSEEEFSAALLHLAKRRGVHN | |
| | VNEVEEDTGNELSTKEQISRNSKALEEKYVAELQLERLKK | |
| | DGEVRGSINRFKTSDYVKEAKQLLKVQKAYHQLDQSFIDT | |
| | YIDLLETRRTYYEGPGEGSPFGWKDIKEWYEMLMGHCTYF | |
| | PEELRSVKYAYNADLYNALNDLNNLVITRDENEKLEYYEK | |
| | FQIIENVFKQKKKPTLKQIAKEILVNEEDIKGYRVTSTGK | |
| | PEFTNLKVYHDIKDITARKEIIENAELLDQIAKILTIYQS | |
| | SEDIQEELTNLNSELTQEEIEQISNLKGYTGTHNLSLKAI | |
| | NLILDELWHTNDNQIAIFNRLKLVPKKVDLSQQKEIPTTL | |
| | VDDFILSPVVKRSFIQSIKVINAIIKKYGLPNDIIIELAR | |
| | EKNSKDAQKMINEMQKRNRQTNERIEEIIRTTGKENAKYL | |
| | IEKIKLHDMQEGKCLYSLEAIPLEDLLNNPFNYEVDHIIP | |
| | RSVSFDNSFNNKVLVKQEENSKKGNRTPFQYLSSSDSKIS | |
| | YETFKKHILNLAKGKGRISKTKKEYLLEERDINRFSVQKD | |
| | FINRNLVDTRYATRGLMNLLRSYFRVNNLDVKVKSINGGF | |
| | TSFLRRKWKFKKERNKGYKHHAEDALIIANADFIFKEWKK | |
| | LDKAKKVMENQMFEEKQAESMPEIETEQEYKEIFITPHQI | |
| | KHIKDFKDYKYSHRVDKKPNRELINDTLYSTRKDDKGNTL | |
| | IVNNLNGLYDKDNDKLKKLINKSPEKLLMYHHDPQTYQKL | |
| | KLIMEQYGDEKNPLYKYYEETGNYLTKYSKKDNGPVIKKI | |
| | KYYGNKLNAHLDITDDYPNSRNKVVKLSLKPYRFDVYLDN | |
| | GVYKFVTVKNLDVIKKENYYEVNSKCYEEAKKLKKISNQA | |
| | EFIASFYNNDLIKINGELYRVIGVNNDLLNRIEVNMIDIT | |
| | YREYLENMNDKRPPRIIKTIASKTQSIKKYSTDILGNLYE | |
| | VKSKKHPQIIKKG | |
| 125 | KRNYILGLAIGITSVGYGIIDYETRDVIDAGVRLFKEANV | dSaCas9 (AA) |
| | ENNEGRRSKRGARRLKRRRRHRIQRVKKLLFDYNLLTDHS | |
| | ELSGINPYEARVKGLSQKLSEEEFSAALLHLAKRRGVHNV | |
| | NEVEEDTGNELSTKEQISRNSKALEEKYVAELQLERLKKD | |
| | GEVRGSINRFKTSDYVKEAKQLLKVQKAYHQLDQSFIDTY | |
| | IDLLETRRTYYEGPGEGSPFGWKDIKEWYEMLMGHCTYFP | |
| | EELRSVKYAYNADLYNALNDLNNLVITRDENEKLEYYEKF | |
| | QIIENVFKQKKKPTLKQIAKEILVNEEDIKGYRVTSTGKP | |
| | EFTNLKVYHDIKDITARKEIIENAELLDQIAKILTIYQSS | |
| | EDIQEELTNLNSELTQEEIEQISNLKGYTGTHNLSLKAIN | |
| | LILDELWHTNDNQIAIFNRLKLVPKKVDLSQQKEIPTTLV | |
| | DDFILSPVVKRSFIQSIKVINAIIKKYGLPNDIIIELARE | |
| | KNSKDAQKMINEMQKRNRQTNERIEEIIRTTGKENAKYLI | |
| | EKIKLHDMQEGKCLYSLEAIPLEDLLNNPFNYEVDHIIPR | |
| | SVSFDNSFNNKVLVKQEEASKKGNRTPFQYLSSSDSKISY | |
| | ETFKKHILNLAKGKGRISKTKKEYLLEERDINRFSVQKDF | |
| | INRNLVDTRYATRGLMNLLRSYFRVNNLDVKVKSINGGFT | |
| | SFLRRKWKFKKERNKGYKHHAEDALIIANADFIFKEWKKL | |
| | DKAKKVMENQMFEEKQAESMPEIETEQEYKEIFITPHQIK | |
| | HIKDFKDYKYSHRVDKKPNRELINDTLYSTRKDDKGNTLI | |
| | VNNLNGLYDKDNDKLKKLINKSPEKLLMYHHDPQTYQKLK | |
| | LIMEQYGDEKNPLYKYYEETGNYLTKYSKKDNGPVIKKIK | |
| | YYGNKLNAHLDITDDYPNSRNKVVKLSLKPYRFDVYLDNG | |
| | VYKFVTVKNLDVIKKENYYEVNSKCYEEAKKLKKISNQAE | |
| | FIASFYNNDLIKINGELYRVIGVNNDLLNRIEVNMIDITY | |
| | REYLENMNDKRPPRIIKTIASKTQSIKKYSTDILGNLYEV | |
| | KSKKHPQIIKKG | |
| 126 | MDKKYSIGLDIGTNSVGWAVITDEYKVPSKKFKVLGNTDR | SpCas9 (AA) |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDH | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGD | |
| 127 | DKKYSIGLAIGTNSVGWAVITDEYKVPSKKFKVLGNTDRH | dSpCas9 (AA) |
| | SIKKNLIGALLFDSGETAEATRLKRTARRRYTRRKNRICY | |
| | LQEIFSNEMAKVDDSFFHRLEESFLVEEDKKHERHPIFGN | |
| | IVDEVAYHEKYPTIYHLRKKLVDSTDKADLRLIYLALAHM | |
| | IKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLAQ | |
| | IGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIE | |
| | KILTFRIPYYVGPLARGNSRFAWMTRKSEETITPWNFEEV | |
| | VDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKII | |
| | KDKDFLDNEENEDILEDIVLTLTLFEDREMIEERLKTYAH | |
| | LFDDKVMKQLKRRRYTGWGRLSRKLINGIRDKQSGKTILD | |
| | FLKSDGFANRNFMQLIHDDSLTFKEDIQKAQVSGQGDSLH | |
| | EHIANLAGSPAIKKGILQTVKVVDELVKVMGRHKPENIVI | |
| | EMARENQTTQKGQKNSRERMKRIEEGIKELGSQILKEHPV | |
| | ENTQLQNEKLYLYYLQNGRDMYVDQELDINRLSDYDVDAI | |
| | VPQSFLKDDSIDNKVLTRSDKNRGKSDNVPSEEVVKKMKN | |
| | YWRQLLNAKLITQRKFDNLTKAERGGLSELDKAGFIKRQL | |
| | VETRQITKHVAQILDSRMNTKYDENDKLIREVKVITLKSK | |
| | LVSDFRKDFQFYKVREINNYHHAHDAYLNAVVGTALIKKY | |
| | PKLESEFVYGDYKVYDVRKMIAKSEQEIGKATAKYFFYSN | |
| | IMNFFKTEITLANGEIRKRPLIETNGETGEIVWDKGRDFA | |
| | TVRKVLSMPQVNIVKKTEVQTGGFSKESILPKRNSDKLIA | |
| | RKKDWDPKKYGGFDSPTVAYSVLVVAKVEKGKSKKLKSVK | |
| | ELLGITIMERSSFEKNPIDFLEAKGYKEVKKDLIIKLPKY | |
| | SLFELENGRKRMLASAGELQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKRYTSTKEVLDATLIHQSITGLYETRID | |
| | LSQLGGD | |
| 128 | MKTPADTGFAFPDWAYKPESSPGSRQIQLWHFILELLRKE | ERF domain (AA) |
| | EYQGVIAWQGDYGEFVIKDPDEVARLWGVRKCKPQMNYDK | |
| | LSRALRYYYNKRILHKTKGKRFTYKFNFNKLVLVNYPFID | |
| | VGLAGGAVPQSAPPVPSGGSHFRFPPSTPSEVLSPTEDPR | |
| | SPPACSSSSSSLFSAVVARRLGRGSVSDCSDGTSELEEPL | |
| | GEDPRARPPGPPDLGAFRGPPLARLPHDPGVFRVYPRPRG | |
| | GPEPLSPFPVSPLAGPGSLLPPQLSPALPMTPTHLAYTPS | |
| | PTLSPMYPSGGGGPSGSGGGSHFSFSPEDMKRYLQAHTQS | |
| | VYNYHLSPRAFLHYPGLVVPQPQRPDKCPLPPMAPETPPV | |
| | PSSASSSSSSSSSPFKFKLQPPPLGRRQRAAGEKAVAGAD | |
| | KSGGSAGGLAEGAGALAPPPPPPQIKVEPISEGESEEVEV | |
| | TDISDEDEEDGEVFKTPRAPPAPPKPEPGEAPGASQCMPL | |
| | KLRFKRRWSEDCRLEGGGGPAGGFEDEGEDKKVRGEGPGE | |
| | AGGPLTPRRVSSDLQHATAQLSLEHRDS | |
| 129 | MERVKMINVQRLLEAAEFLERRERECEHGYASSFPSMPSP | MXI1 domain |
| | RLQHSKPPRRLSRAQKHSSGSSNTSTANRSTHNELEKNRR | (AA) |
| | AHLRLCLERLKVLIPLGPDCTRHTTLGLLNKAKAHIKKLE | |
| | EAERKSQHQLENLEREQRFLKWRLEQLQGPQEMERIRMDS | |
| | IGSTISSDRSDSEREEIEVDVESTEFSHGEVDNISTTSIS | |
| | DIDDHSSLPSIGSDEGYSSASVKLSFTS | |
| 130 | ACCTACGGGCTGCTGCGGCGGCGAGAGGACTGGCCCTCCC | DNMT3A (nt) |
| | GGCTCCAGATGTTCTTCGCTAATAACCACGACCAGGAATT | |
| | TGACCCTCCAAAGGTTTACCCACCTGTCCCAGCTGAGAAG | |
| | AGGAAGCCCATCCGGGTGCTGTCTCTCTTTGATGGAATCG | |
| | CTACAGGGCTCCTGGTGCTGAAGGACTTGGGCATTCAGGT | |
| | GGACCGCTACATTGCCTCGGAGGTGTGTGAGGACTCCATC | |
| | ACGGTGGGCATGGTGCGGCACCAGGGGAAGATCATGTACG | |
| | TCGGGGACGTCCGCAGCGTCACACAGAAGCATATCCAGGA | |
| | GTGGGGCCCATTCGATCTGGTGATTGGGGGCAGTCCCTGC | |
| | AATGACCTCTCCATCGTCAACCCTGCTCGCAAGGGCCTCT | |
| | ACGAGGGCACTGGCCGGCTCTTCTTTGAGTTCTACCGCCT | |
| | CCTGCATGATGCGCGGCCCAAGGAGGGAGATGATCGCCCC | |
| | TTCTTCTGGCTCTTTGAGAATGTGGTGGCCATGGGCGTTA | |
| | GTGACAAGAGGGACATCTCGCGATTTCTCGAGTCCAACCC | |
| | TGTGATGATTGATGCCAAAGAAGTGTCAGCTGCACACAGG | |
| | GCCCGCTACTTCTGGGGTAACCTTCCCGGTATGAACAGGC | |
| | CGTTGGCATCCACTGTGAATGATAAGCTGGAGCTGCAGGA | |
| | GTGTCTGGAGCATGGCAGGATAGCCAAGTTCAGCAAAGTG | |
| | AGGACCATTACTACGAGGTCAAACTCCATAAAGCAGGGCA | |
| | AAGACCAGCATTTTCCTGTCTTCATGAATGAGAAAGAGGA | |
| | CATCTTATGGTGCACTGAAATGGAAAGGGTATTTGGTTTC | |
| | CCAGTCCACTATACTGACGTATCCAACATGAGCCGCTTGG | |
| | CGAGGCAGAGACTGCTGGGCCGGTCATGGAGCGTGCCAGT | |
| | CATCCGCCACCTCTTCGCTCCGCTGAAGGAGTATTTTGCG | |
| | TGTGTG | |
| 131 | TYGLLRRREDWPSRLQMFFANNHDQEFDPPKVYPPVPAEK | DNMT3A (AA) |
| | RKPIRVLSLFDGIATGLLVLKDLGIQVDRYIASEVCEDSI | |
| | TVGMVRHQGKIMYVGDVRSVTQKHIQEWGPFDLVIGGSPC | |
| | NDLSIVNPARKGLYEGTGRLFFEFYRLLHDARPKEGDDRP | |
| | FFWLFENVVAMGVSDKRDISRFLESNPVMIDAKEVSAAHR | |
| | ARYFWGNLPGMNRPLASTVNDKLELQECLEHGRIAKFSKV | |
| | RTITTRSNSIKQGKDQHFPVFMNEKEDILWCTEMERVFGF | |
| | PVHYTDVSNMSRLARQRLLGRSWSVPVIRHLFAPLKEYFA | |
| | CV | |
| 132 | ATGGCGGCCATCCCAGCCCTGGACCCAGAGGCCGAGCCCA | DNMT3L - Human |
| | GCATGGACGTGATTTTGGTGGGATCCAGTGAGCTCTCAAG | (nt) |
| | CTCCGTTTCACCCGGGACAGGCAGAGATCTTATTGCATAT | |
| | GAAGTCAAGGCTAACCAGCGAAATATAGAAGACATCTGCA | |
| | TCTGCTGCGGAAGTCTCCAGGTTCACACACAGCACCCTCT | |
| | GTTTGAGGGAGGGATCTGCGCCCCATGTAAGGACAAGTTC | |
| | CTGGATGCCCTCTTCCTGTACGACGATGACGGGTACCAAT | |
| | CCTACTGCTCCATCTGCTGCTCCGGAGAAACGCTGCTCAT | |
| | CTGCGGAAACCCTGATTGCACCCGATGCTACTGCTTCGAG | |
| | TGTGTGGATAGCCTGGTCGGCCCCGGGACCTCGGGGAAGG | |
| | TGCACGCCATGAGCAACTGGGTGTGCTACCTGTGCCTGCC | |
| | GTCCTCCCGAAGCGGGCTGCTGCAGCGTCGGAGGAAGTGG | |
| | CGCAGCCAGCTCAAGGCCTTCTACGACCGAGAGTCGGAGA | |
| | ATCCCCTTGAGATGTTCGAAACCGTGCCTGTGTGGAGGAG | |
| | ACAGCCAGTCCGGGTGCTGTCCCTTTTTGAAGACATCAAG | |
| | AAAGAGCTGACGAGTTTGGGCTTTTTGGAAAGTGGTTCTG | |
| | ACCCGGGACAACTGAAGCATGTGGTTGATGTCACAGACAC | |
| | AGTGAGGAAGGATGTGGAGGAGTGGGGACCCTTCGATCTT | |
| | GTGTACGGCGCCACACCTCCCCTGGGCCACACCTGTGACC | |
| | GTCCTCCCAGCTGGTACCTGTTCCAGTTCCACCGGCTCCT | |
| | GCAGTACGCACGGCCCAAGCCAGGCAGCCCCAGGCCCTTC | |
| | TTCTGGATGTTCGTGGACAATCTGGTGCTGAACAAGGAAG | |
| | ACCTGGACGTCGCATCTCGCTTCCTGGAGATGGAGCCAGT | |
| | CACCATCCCAGATGTCCACGGCGGATCCTTGCAGAATGCT | |
| | GTCCGCGTGTGGAGCAACATCCCAGCCATAAGGAGCAGGC | |
| | ACTGGGCTCTGGTTTCGGAAGAAGAATTGTCCCTGCTGGC | |
| | CCAGAACAAGCAGAGCTCGAAGCTCGCGGCCAAGTGGCCC | |
| | ACCAAGCTGGTGAAGAACTGCTTTCTCCCCCTAAGAGAAT | |
| | ATTTCAAGTATTTTTCAACAGAACTCACTTCCTCTTTA | |
| 133 | MAAIPALDPEAEPSMDVILVGSSELSSSVSPGTGRDLIAY | DNMT3L - Human |
| | EVKANQRNIEDICICCGSLQVHTQHPLFEGGICAPCKDKF | (AA) |
| | LDALFLYDDDGYQSYCSICCSGETLLICGNPDCTRCYCFE | |
| | CVDSLVGPGTSGKVHAMSNWVCYLCLPSSRSGLLQRRRKW | |
| | RSQLKAFYDRESENPLEMFETVPVWRRQPVRVLSLFEDIK | |
| | KELTSLGFLESGSDPGQLKHVVDVTDTVRKDVEEWGPFDL | |
| | VYGATPPLGHTCDRPPSWYLFQFHRLLQYARPKPGSPRPF | |
| | FWMFVDNLVLNKEDLDVASRFLEMEPVTIPDVHGGSLQNA | |
| | VRVWSNIPAIRSRHWALVSEEELSLLAQNKQSSKLAAKWP | |
| | TKLVKNCFLPLREYFKYFSTELTSSL | |
| 134 | AACCATGACCAGGAATTTGACCCCCCAAAGGTTTACCCAC | DNMT3A/L v1 |
| | CTGTGCCAGCTGAGAAGAGGAAGCCCATCCGCGTGCTGTC | (nt) |
| | TCTCTTTGATGGGATTGCTACAGGGCTCCTGGTGCTGAAG | |
| | GACCTGGGCATCCAAGTGGACCGCTACATTGCCTCCGAGG | |
| | TGTGTGAGGACTCCATCACGGTGGGCATGGTGCGGCACCA | |
| | GGGAAAGATCATGTACGTCGGGGACGTCCGCAGCGTCACA | |
| | CAGAAGCATATCCAGGAGTGGGGCCCATTCGACCTGGTGA | |
| | TTGGAGGCAGTCCCTGCAATGACCTCTCCATTGTCAACCC | |
| | TGCCCGCAAGGGACTTTATGAGGGTACTGGCCGCCTCTTC | |
| | TTTGAGTTCTACCGCCTCCTGCATGATGCGCGGCCCAAGG | |
| | AGGGAGATGATCGCCCCTTCTTCTGGCTCTTTGAGAATGT | |
| | GGTGGCCATGGGCGTTAGTGACAAGAGGGACATCTCGCGA | |
| | TTTCTTGAGTCTAACCCCGTGATGATTGACGCCAAAGAAG | |
| | TGTCTGCTGCACACAGGGCCCGTTACTTCTGGGGTAACCT | |
| | TCCTGGCATGAACAGGCCTTTGGCATCCACTGTGAATGAT | |
| | AAGCTGGAGCTGCAAGAGTGTCTGGAGCACGGCAGAATAG | |
| | CCAAGTTCAGCAAAGTGAGGACCATTACCACCAGGTCAAA | |
| | CTCTATAAAGCAGGGCAAAGACCAGCATTTCCCCGTCTTC | |
| | ATGAACGAGAAGGAGGACATCCTGTGGTGCACTGAAATGG | |
| | AAAGGGTGTTTGGCTTCCCCGTCCACTACACAGACGTCTC | |
| | CAACATGAGCCGCTTGGCGAGGCAGAGACTGCTGGGCCGA | |
| | TCGTGGAGCGTGCCGGTCATCCGCCACCTCTTCGCTCCGC | |
| | TGAAGGAATATTTTGCTTGTGTGTCTAGCGGCAATAGTAA | |
| | CGCTAACAGCCGCGGGCCGAGCTTCAGCAGCGGCCTGGTG | |
| | CCGTTAAGCTTGCGCGGCAGCCATATGGGCCCTATGGAGA | |
| | TATACAAGACAGTGTCTGCATGGAAGAGACAGCCAGTGCG | |
| | GGTACTGAGCCTCTTCAGAAACATCGACAAGGTACTAAAG | |
| | AGTTTGGGCTTCTTGGAAAGCGGTTCTGGTTCTGGGGGAG | |
| | GAACGCTGAAGTACGTGGAAGATGTCACAAATGTCGTGAG | |
| | GAGAGACGTGGAGAAATGGGGCCCCTTTGACCTGGTGTAC | |
| | GGCTCGACGCAGCCCCTAGGCAGCTCTTGTGATCGCTGTC | |
| | CCGGCTGGTACATGTTCCAGTTCCACCGGATCCTGCAGTA | |
| | TGCGCTGCCTCGCCAGGAGAGTCAGCGGCCCTTCTTCTGG | |
| | ATATTCATGGACAATCTGCTGCTGACTGAGGATGACCAAG | |
| | AGACAACTACCCGCTTCCTTCAGACAGAGGCTGTGACCCT | |
| | CCAGGATGTCCGTGGCAGAGACTACCAGAATGCTATGCGG | |
| | GTGTGGAGCAACATTCCAGGGCTGAAGAGCAAGCATGCGC | |
| | CCCTGACCCCAAAGGAAGAAGAGTATCTGCAAGCCCAAGT | |
| | CAGAAGCAGGAGCAAGCTGGACGCCCCGAAAGTTGACCTC | |
| | CTGGTGAAGAACTGCCTTCTCCCGCTGAGAGAGTACTTCA | |
| | AGTATTTTTCTCAAAACTCACTTCCTCTT | |
| 135 | NHDQEFDPPKVYPPVPAEKRKPIRVLSLFDGIATGLLVLK | DNMT3A/L v1 |
| | DLGIQVDRYIASEVCEDSITVGMVRHQGKIMYVGDVRSVT | (AA) |
| | QKHIQEWGPFDLVIGGSPCNDLSIVNPARKGLYEGTGRLF | |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPL | |
| 136 | AACCACGATCAGGAGTTTGACCCCCCTAAGGTGTACCCAC | DNMT3A/L v2 |
| | CCGTGCCAGCCGAGAAGAGGAAGCCCATCCGCGTGCTGTC | (nt) |
| | CCTGTTCGACGGCATCGCCACAGGCCTGCTGGTGCTGAAG | |
| | GATCTGGGCATCCAGGTGGACAGATATATCGCCTCCGAGG | |
| | TGTGCGAGGATTCTATCACCGTGGGCATGGTGAGGCACCA | |
| | GGGCAAGATCATGTACGTGGGCGACGTGCGCAGCGTGACA | |
| | CAGAAGCACATCCAGGAGTGGGGACCCTTCGACCTGGTCA | |
| | TCGGAGGCAGCCCCTGTAATGACCTGTCCATCGTGAACCC | |
| | TGCAAGGAAGGGCCTGTATGAGGGAACCGGCAGACTGTTC | |
| | TTTGAGTTCTACAGGCTGCTGCACGACGCCCGCCCTAAGG | |
| | AGGGCGATGACAGGCCATTCTTTTGGCTGTTTGAGAACGT | |
| | GGTGGCCATGGGCGTGAGCGACAAGCGGGATATCTCCAGA | |
| | TTCCTGGAGTCTAATCCCGTGATGATCGATGCAAAGGAGG | |
| | TGTCTGCCGCACACAGGGCAAGGTACTTTTGGGGAAATCT | |
| | GCCTGGCATGAACCGCCCACTGGCCAGCACCGTGAACGAC | |
| | AAGCTGGAGCTGCAGGAGTGCCTGGAGCACGGAAGGATCG | |
| | CCAAGTTCTCCAAGGTGCGGACAATCACCACAAGATCTAA | |
| | CAGCATCAAGCAGGGCAAGGATCAGCACTTCCCCGTGTTC | |
| | ATGAATGAGAAGGAGGACATCCTGTGGTGTACCGAGATGG | |
| | AGCGCGTGTTCGGCTTTCCAGTGCACTATACAGACGTGAG | |
| | CAATATGAGCCGGCTGGCAAGGCAGAGACTGCTGGGCCGG | |
| | TCCTGGTCTGTGCCAGTGATCAGACACCTGTTCGCCCCCC | |
| | TGAAGGAGTACTTTGCCTGCGTGTCTAGCGGCAACTCTAA | |
| | TGCCAACAGCAGAGGCCCTTCCTTTTCCTCTGGCCTGGTG | |
| | CCACTGTCTCTGAGGGGCAGCCACATGGGCCCCATGGAGA | |
| | TCTACAAGACCGTGTCCGCCTGGAAGAGGCAGCCTGTGCG | |
| | CGTGCTGTCTCTGTTCCGCAACATCGACAAGGTGCTGAAG | |
| | AGCCTGGGCTTTCTGGAGAGCGGATCCGGATCTGGAGGAG | |
| | GCACCCTGAAGTATGTGGAGGATGTGACAAATGTGGTGCG | |
| | GAGAGATGTGGAGAAGTGGGGCCCCTTCGATCTGGTGTAC | |
| | GGATCCACCCAGCCACTGGGAAGCTCCTGCGATAGGTGTC | |
| | CAGGATGGTATATGTTCCAGTTTCACAGAATCCTGCAGTA | |
| | CGCACTGCCAAGGCAGGAGAGCCAGCGCCCTTTCTTTTGG | |
| | ATCTTTATGGACAACCTGCTGCTGACAGAGGATGACCAGG | |
| | AGACAACAACCCGCTTCCTGCAGACAGAGGCAGTGACCCT | |
| | GCAGGATGTGAGGGGACGCGACTATCAGAATGCCATGCGG | |
| | GTGTGGTCTAACATCCCTGGCCTGAAGAGCAAGCACGCCC | |
| | CCCTGACCCCTAAGGAGGAGGAGTACCTGCAGGCCCAGGT | |
| | GCGGAGCAGATCCAAGCTGGATGCCCCTAAGGTGGACCTG | |
| | CTGGTGAAGAATTGTCTGCTGCCACTGCGGGAGTACTTCA | |
| | AGTACTTTAGTCAGAATAGCCTGCCACTG | |
| 137 | NHDQEFDPPKVYPPVPAEKRKPIRVLSLFDGIATGLLVLK | DNMT3A/L v2 |
| | DLGIQVDRYIASEVCEDSITVGMVRHQGKIMYVGDVRSVT | (AA) |
| | QKHIQEWGPFDLVIGGSPCNDLSIVNPARKGLYEGTGRLF | |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPL | |
| 138 | PKKKRKVGIHGVPAADKKYSIGLAIGTNSVGWAVITDEYK | dSpCas9-KRAB |
| | VPSKKFKVLGNTDRHSIKKNLIGALLFDSGETAEATRLKR | (AA) |
| | TARRRYTRRKNRICYLQEIFSNEMAKVDDSFFHRLEESFL | |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDKRPAATKKAGQAKKKKAS | |
| | DAKSLTAWSRTLVTFKDVFVDFTREEWKLLDTAQQILYRN | |
| | VMLENYKNLVSLGYQLTKPDVILRLEKGEEPWLVEREIHQ | |
| | ETHPDSETAFEIKSSVPKKKRKV | |
| 139 | NHDQEFDPPKVYPPVPAEKRKPIRVLSLFDGIATGLLVLK | dSpCas9-KRAB- |
| | DLGIQVDRYIASEVCEDSITVGMVRHQGKIMYVGDVRSVT | DNTM3A/L (AA) |
| | QKHIQEWGPFDLVIGGSPCNDLSIVNPARKGLYEGTGRLF | |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLEASGSGRASPGIPGSTR | |
| | NPKKKRKVGIHGVPAADKKYSIGLAIGTNSVGWAVITDEY | |
| | KVPSKKFKVLGNTDRHSIKKNLIGALLFDSGETAEATRLK | |
| | RTARRRYTRRKNRICYLQEIFSNEMAKVDDSFFHRLEESF | |
| | LVEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLENL | |
| | IAQLPGEKKNGLFGNLIALSLGLTPNFKSNFDLAEDAKLQ | |
| | LSKDTYDDDLDNLLAQIGDQYADLFLAAKNLSDAILLSDI | |
| | LRVNTEITKAPLSASMIKRYDEHHQDLTLLKALVRQQLPE | |
| | KYKEIFFDQSKNGYAGYIDGGASQEEFYKFIKPILEKMDG | |
| | TEELLVKLNREDLLRKQRTFDNGSIPHQIHLGELHAILRR | |
| | QEDFYPFLKDNREKIEKILTFRIPYYVGPLARGNSRFAWM | |
| | TRKSEETITPWNFEEVVDKGASAQSFIERMTNFDKNLPNE | |
| | KVLPKHSLLYEYFTVYNELTKVKYVTEGMRKPAFLSGEQK | |
| | KAIVDLLFKTNRKVTVKQLKEDYFKKIECFDSVEISGVED | |
| | RFNASLGTYHDLLKIIKDKDFLDNEENEDILEDIVLTLTL | |
| | FEDREMIEERLKTYAHLFDDKVMKQLKRRRYTGWGRLSRK | |
| | LINGIRDKQSGKTILDFLKSDGFANRNFMQLIHDDSLTFK | |
| | EDIQKAQVSGQGDSLHEHIANLAGSPAIKKGILQTVKVVD | |
| | ELVKVMGRHKPENIVIEMARENQTTQKGQKNSRERMKRIE | |
| | EGIKELGSQILKEHPVENTQLQNEKLYLYYLQNGRDMYVD | |
| | QELDINRLSDYDVDAIVPQSFLKDDSIDNKVLTRSDKNRG | |
| | KSDNVPSEEVVKKMKNYWRQLLNAKLITQRKFDNLTKAER | |
| | GGLSELDKAGFIKRQLVETRQITKHVAQILDSRMNTKYDE | |
| | NDKLIREVKVITLKSKLVSDFRKDFQFYKVREINNYHHAH | |
| | DAYLNAVVGTALIKKYPKLESEFVYGDYKVYDVRKMIAKS | |
| | EQEIGKATAKYFFYSNIMNFFKTEITLANGEIRKRPLIET | |
| | NGETGEIVWDKGRDFATVRKVLSMPQVNIVKKTEVQTGGF | |
| | SKESILPKRNSDKLIARKKDWDPKKYGGFDSPTVAYSVLV | |
| | VAKVEKGKSKKLKSVKELLGITIMERSSFEKNPIDFLEAK | |
| | GYKEVKKDLIIKLPKYSLFELENGRKRMLASAGELQKGNE | |
| | LALPSKYVNFLYLASHYEKLKGSPEDNEQKQLFVEQHKHY | |
| | LDEIIEQISEFSKRVILADANLDKVLSAYNKHRDKPIREQ | |
| | AENIIHLFTLTNLGAPAAFKYFDTTIDRKRYTSTKEVLDA | |
| | TLIHQSITGLYETRIDLSQLGGDSGGKRPAATKKAGQAKK | |
| | KKASDAKSLTAWSRTLVTFKDVFVDFTREEWKLLDTAQQI | |
| | LYRNVMLENYKNLVSLGYQLTKPDVILRLEKGEEPWLVER | |
| | EIHQETHPDSETAFEIKSSVPKKKRKV | |
| 140 | MNHDQEFDPPKVYPPVPAEKRKPIRVLSLFDGIATGLLVL | DNMT3A/L- |
| | KDLGIQVDRYIASEVCEDSITVGMVRHQGKIMYVGDVRSV | XTEN80-dSpCas9- |
| | TQKHIQEWGPFDLVIGGSPCNDLSIVNPARKGLYEGTGRL | KRAB (AA) |
| | FFEFYRLLHDARPKEGDDRPFFWLFENVVAMGVSDKRDIS | |
| | RFLESNPVMIDAKEVSAAHRARYFWGNLPGMNRPLASTVN | |
| | DKLELQECLEHGRIAKFSKVRTITTRSNSIKQGKDQHFPV | |
| | FMNEKEDILWCTEMERVFGFPVHYTDVSNMSRLARQRLLG | |
| | RSWSVPVIRHLFAPLKEYFACVSSGNSNANSRGPSFSSGL | |
| | VPLSLRGSHMGPMEIYKTVSAWKRQPVRVLSLFRNIDKVL | |
| | KSLGFLESGSGSGGGTLKYVEDVTNVVRRDVEKWGPFDLV | |
| | YGSTQPLGSSCDRCPGWYMFQFHRILQYALPRQESQRPFF | |
| | WIFMDNLLLTEDDQETTTRFLQTEAVTLQDVRGRDYQNAM | |
| | RVWSNIPGLKSKHAPLTPKEEEYLQAQVRSRSKLDAPKVD | |
| | LLVKNCLLPLREYFKYFSQNSLPLGGPSSGAPPPSGGSPA | |
| | GSPTSTEEGTSESATPESGPGTSTEPSEGSAPGSPAGSPT | |
| | STEEGTSTEPSEGSAPGTSTEPSEVNPKKKRKVGIHGVPA | |
| | ADKKYSIGLAIGTNSVGWAVITDEYKVPSKKFKVLGNTDR | |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASDAKSLTAWSRT | |
| | LVTFKDVFVDFTREEWKLLDTAQQILYRNVMLENYKNLVS | |
| | LGYQLTKPDVILRLEKGEEPWLVEREIHQETHPDSETAFE | |
| | IKSSVPKKKRKV | |
| 141 | SNHDQEFDPPKVYPPVPAEKRKPIRVLSLFDGIATGLLVL | DNMT3A/L(CRIS |
| | KDLGIQVDRYIASEVCEDSITVGMVRHQGKIMYVGDVRSV | PROFF)-XTEN80- |
| | TQKHIQEWGPFDLVIGGSPCNDLSIVNPARKGLYEGTGRL | dSpCas9-KRAB |
| | FFEFYRLLHDARPKEGDDRPFFWLFENVVAMGVSDKRDIS | (AA) |
| | RFLESNPVMIDAKEVSAAHRARYFWGNLPGMNRPLASTVN | |
| | DKLELQECLEHGRIAKFSKVRTITTRSNSIKQGKDQHFPV | |
| | FMNEKEDILWCTEMERVFGFPVHYTDVSNMSRLARQRLLG | |
| | RSWSVPVIRHLFAPLKEYFACVSSGNSNANSRGPSFSSGL | |
| | VPLSLRGSHMGPMEIYKTVSAWKRQPVRVLSLFRNIDKVL | |
| | KSLGFLESGSGSGGGTLKYVEDVTNVVRRDVEKWGPFDLV | |
| | YGSTQPLGSSCDRCPGWYMFQFHRILQYALPRQESQRPFF | |
| | WIFMDNLLLTEDDQETTTRFLQTEAVTLQDVRGRDYQNAM | |
| | RVWSNIPGLKSKHAPLTPKEEEYLQAQVRSRSKLDAPKVD | |
| | LLVKNCLLPLREYFKYFSQNSLPLGGPSSGAPPPSGGSPA | |
| | GSPTSTEEGTSESATPESGPGTSTEPSEGSAPGSPAGSPT | |
| | STEEGTSTEPSEGSAPGTSTEPSEVNPKKKRKVGIHGVPA | |
| | ADKKYSIGLAIGTNSVGWAVITDEYKVPSKKFKVLGNTDR | |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASDAKSLTAWSRT | |
| | LVTFKDVFVDFTREEWKLLDTAQQILYRNVMLENYKNLVS | |
| | LGYQLTKPDVILRLEKGEEPWLVEREIHQETHPDSETAFE | |
| | IKSSVPKKKRKV | |
| 142 | DALDDFDLDMLGSDALDDFDLDMLGSDALDDFDLDMLGSD | VP64 (AA) |
| | ALDDFDLDM | |
| 143 | DALDDFDLDMLGSDALDDFDLDMLGSDALDDFDLDMLGSD | VP64-dSpCas9- |
| | ALDDFDLDMVNPKKKRKVGIHGVPAADKKYSIGLAIGTNS | NLS-VP64 (AA) |
| | VGWAVITDEYKVPSKKFKVLGNTDRHSIKKNLIGALLFDS | |
| | GETAEATRLKRTARRRYTRRKNRICYLQEIFSNEMAKVDD | |
| | SFFHRLEESFLVEEDKKHERHPIFGNIVDEVAYHEKYPTI | |
| | YHLRKKLVDSTDKADLRLIYLALAHMIKFRGHFLIEGDLN | |
| | PDNSDVDKLFIQLVQTYNQLFEENPINASGVDAKAILSAR | |
| | LSKSRRLENLIAQLPGEKKNGLFGNLIALSLGLTPNFKSN | |
| | FDLAEDAKLQLSKDTYDDDLDNLLAQIGDQYADLFLAAKN | |
| | LSDAILLSDILRVNTEITKAPLSASMIKRYDEHHQDLTLL | |
| | KALVRQQLPEKYKEIFFDQSKNGYAGYIDGGASQEEFYKF | |
| | IKPILEKMDGTEELLVKLNREDLLRKQRTFDNGSIPHQIH | |
| | LGELHAILRRQEDFYPFLKDNREKIEKILTFRIPYYVGPL | |
| | ARGNSRFAWMTRKSEETITPWNFEEVVDKGASAQSFIERM | |
| | TNFDKNLPNEKVLPKHSLLYEYFTVYNELTKVKYVTEGMR | |
| | KPAFLSGEQKKAIVDLLFKTNRKVTVKQLKEDYFKKIECF | |
| | DSVEISGVEDRFNASLGTYHDLLKIIKDKDFLDNEENEDI | |
| | LEDIVLTLTLFEDREMIEERLKTYAHLFDDKVMKQLKRRR | |
| | YTGWGRLSRKLINGIRDKQSGKTILDFLKSDGFANRNFMQ | |
| | LIHDDSLTFKEDIQKAQVSGQGDSLHEHIANLAGSPAIKK | |
| | GILQTVKVVDELVKVMGRHKPENIVIEMARENQTTQKGQK | |
| | NSRERMKRIEEGIKELGSQILKEHPVENTQLQNEKLYLYY | |
| | LQNGRDMYVDQELDINRLSDYDVDAIVPQSFLKDDSIDNK | |
| | VLTRSDKNRGKSDNVPSEEVVKKMKNYWRQLLNAKLITQR | |
| | KFDNLTKAERGGLSELDKAGFIKRQLVETRQITKHVAQIL | |
| | DSRMNTKYDENDKLIREVKVITLKSKLVSDFRKDFQFYKV | |
| | REINNYHHAHDAYLNAVVGTALIKKYPKLESEFVYGDYKV | |
| | YDVRKMIAKSEQEIGKATAKYFFYSNIMNFFKTEITLANG | |
| | EIRKRPLIETNGETGEIVWDKGRDFATVRKVLSMPQVNIV | |
| | KKTEVQTGGFSKESILPKRNSDKLIARKKDWDPKKYGGFD | |
| | SPTVAYSVLVVAKVEKGKSKKLKSVKELLGITIMERSSFE | |
| | KNPIDFLEAKGYKEVKKDLIIKLPKYSLFELENGRKRMLA | |
| | SAGELQKGNELALPSKYVNFLYLASHYEKLKGSPEDNEQK | |
| | QLFVEQHKHYLDEIIEQISEFSKRVILADANLDKVLSAYN | |
| | KHRDKPIREQAENIIHLFTLTNLGAPAAFKYFDTTIDRKR | |
| | YTSTKEVLDATLIHQSITGLYETRIDLSQLGGDKRPAATK | |
| | KAGQAKKKKASRADALDDFDLDMLGSDALDDFDLDMLGSD | |
| | ALDDFDLDMLGSDALDDFDLDML | |
| 144 | CTGACTGCAGCATTTCACAC | CD28_1 target site |
| 145 | GCTGCAGTCAGGATGCCTTG | CD28_2 target site |
| 146 | CCTTGATCATGTGCCCTAAG | CD28_3 target site |
| 147 | CGCAGTAGCGGCCCGCGAGT | EOMES_1 target |
| | | site |
| 148 | CGGGCCGCTACTGCGCGTAC | EOMES_2 target |
| | | site |
| 149 | GCTACCCACCTGCCGACTCG | EOMES_3 target |
| | | site |
| 150 | TCCAGTCACCCCAACCCAGT | LCP2_1 target site |
| 151 | GGTGCTACACTGTGCAGACA | LCP2_2 target site |
| 152 | CAGGGTTGGTAATTCCCCAC | LCP2_3 target site |
| 153 | CAGGGCCGAGGTGGCGGAGT | TBX21_1 target |
| | | site |
| 154 | CTCGCTTCTCTCCACCATGG | TBX21_2 target |
| | | site |
| 155 | CTAGGCAAATTCTACGCTCT | TBX21_3 target |
| | | site |
| 156 | CCTGTAACTGTCGCCCTGAG | VAV1_4 target site |
| 157 | CUGACUGCAGCAUUUCACAC | CD28_1 gRNA |
| 158 | GCUGCAGUCAGGAUGCCUUG | CD28_2 gRNA |
| 159 | CCUUGAUCAUGUGCCCUAAG | CD28_3 gRNA |
| 160 | CGCAGUAGCGGCCCGCGAGU | EOMES_1 gRNA |
| 161 | CGGGCCGCUACUGCGCGUAC | EOMES_2 gRNA |
| 162 | GCUACCCACCUGCCGACUCG | EOMES_3 gRNA |
| 163 | UCCAGUCACCCCAACCCAGU | LCP2_1 gRNA |
| 164 | GGUGCUACACUGUGCAGACA | LCP2_2 gRNA |
| 165 | CAGGGUUGGUAAUUCCCCAC | LCP2_3 gRNA |
| 166 | CAGGGCCGAGGUGGCGGAGU | TBX21_1 gRNA |
| 167 | CUCGCUUCUCUCCACCAUGG | TBX21_2 gRNA |
| 168 | CUAGGCAAAUUCUACGCUCU | TBX21_3 gRNA |
| 169 | CCUGUAACUGUCGCCCUGAG | VAV1_4 gRNA |
| 170 | CCAGGCCTGTGTCGAGTGGG | VAV1_5 target site |
| 171 | CCAGGCCUGUGUCGAGUGGG | VAV1_5 gRNA |
| 172 | ACTCACGCTGGAAGTCACAT | BATF_1 target site |
| 173 | AAGTCCGTCTTCTGTCAACA | BATF_2 target site |
| 174 | GCAGAGGGACTGCTCCCCAA | BATF_3 target site |
| 175 | CCGCTTCGGGGACTGTCACT | IRF4_1 target site |
| 176 | ACTTTGCAAGCCGAGAGCCG | IRF4_2 target site |
| 177 | GTCCAACCCCCGGCCCCCAC | IRF4_3 target site |
| 178 | ACUCACGCUGGAAGUCACAU | BATF_1 gRNA |
| | | spacer |
| 179 | AAGUCCGUCUUCUGUCAACA | BATF_2 gRNA |
| | | spacer |
| 180 | GCAGAGGGACUGCUCCCCAA | BATF_3 gRNA |
| | | spacer |
| 181 | CCGCUUCGGGGACUGUCACU | IRF4_1 gRNA |
| | | spacer |
| 182 | ACUUUGCAAGCCGAGAGCCG | IRF4_2 gRNA |
| | | spacer |
| 183 | GUCCAACCCCCGGCCCCCAC | IRF4_3 gRNA |
| | | spacer |
| 184 | GGCTGGGACGCAGGGGTAAC | LAT_1 target site |
| 185 | CCACCCCAGGCACTCACCAA | LAT_2 target site |
| 186 | CTGTGGTGAGCGCCGGGCGA | LAT_3 target site |
| 187 | AAAGACATCGGCTCCAACAG | LCP2_4 target site |
| 188 | AGCCGTTGCTTTCTGGGATC | LCP2_5 target site |
| 189 | TCGTCAGGACAAAGATGCTC | CD28_4 target site |
| 190 | CGTGGATGACGGAGACTCTC | CD28_5 target site |
| 191 | CTGAGAGTCTCCGTCATCCA | CD28_6 target site |
| 192 | GGCUGGGACGCAGGGGUAAC | LAT_1 gRNA |
| | | spacer |
| 193 | CCACCCCAGGCACUCACCAA | LAT_2 gRNA |
| | | spacer |
| 194 | CUGUGGUGAGCGCCGGGCGA | LAT_3 gRNA |
| | | spacer |
| 195 | AAAGACAUCGGCUCCAACAG | LCP2_4 gRNA |
| | | spacer |
| 196 | AGCCGUUGCUUUCUGGGAUC | LCP2_5 gRNA |
| | | spacer |
| 197 | UCGUCAGGACAAAGAUGCUC | CD28_4 gRNA |
| | | spacer |
| 198 | CGUGGAUGACGGAGACUCUC | CD28_5 gRNA |
| | | spacer |
| 199 | CUGAGAGUCUCCGUCAUCCA | CD28_6 gRNA |
| | | spacer |
| 200 | GACCCGCTCAGTACGGAGTT | FAS_1 target site |
| 201 | TCCCCAACTCCGTACTGAGC | FAS_2 target site |
| 202 | GTTGGTGGACCCGCTCAGTA | FAS_3 target site |
| 203 | GGACCCGCTCAGTACGGAGT | FAS_4 target site |
| 204 | ACCCGCTCAGTACGGAGTTG | FAS_5 target site |
| 205 | GAAGCAGTGGTTAAGCCGGA | FAS_6 target site |
| 206 | CGCGCGGCGGCCCAGGAGGG | Fli1_1 target site |
| 207 | GCGCTCGCAGGGGGCACGCA | Fli1_2 target site |
| 208 | ACAACAACAAACGTGCACAG | Fli1_3 target site |
| 209 | GAGGGCAGGGCGCTCGCAGG | Fli1_4 target site |
| 210 | AAACGTGCACAGGGGAGTGA | Fli1_5 target site |
| 211 | GAGCGAAAGAGACAGTTAAC | Fli1_6 target site |
| 212 | GACCCGCUCAGUACGGAGUU | FAS_1 gRNA |
| | | spacer |
| 213 | UCCCCAACUCCGUACUGAGC | FAS_2 gRNA |
| | | spacer |
| 214 | GUUGGUGGACCCGCUCAGUA | FAS_3 gRNA |
| | | spacer |
| 215 | GGACCCGCUCAGUACGGAGU | FAS_4 gRNA |
| | | spacer |
| 216 | ACCCGCUCAGUACGGAGUUG | FAS_5 gRNA |
| | | spacer |
| 217 | GAAGCAGUGGUUAAGCCGGA | FAS_6 gRNA |
| | | spacer |
| 218 | CGCGCGGCGGCCCAGGAGGG | Fli1_1 gRNA |
| | | spacer |
| 219 | GCGCUCGCAGGGGGCACGCA | Fli1_2 gRNA |
| | | spacer |
| 220 | ACAACAACAAACGUGCACAG | Fli1_3 gRNA |
| | | spacer |
| 221 | GAGGGCAGGGCGCUCGCAGG | Fli1_4 gRNA |
| | | spacer |
| 222 | AAACGUGCACAGGGGAGUGA | Fli1_5 gRNA |
| | | spacer |
| 223 | GAGCGAAAGAGACAGUUAAC | Fli1_6 gRNA |
| | | spacer |
| 224 | NGG | PAM - SpCas9 |
| 225 | NNGRRT | PAM - SaCas9 |
| 226 | NNNNGATT | PAM |
| 227 | NNNNRYAC | PAM |
| 228 | NNAGAAW | PAM |
| 229 | NAAAAC | PAM |
| 230 | TTTV | PAM |
| 231 | NGAN | PAM |
| 232 | NGNG | PAM |
| 233 | NGAG | PAM |
| 234 | NGCG | PAM |
| 235 | RTLVTFKDVFVDFTREEWKLLDTAQQILYRNVMLENYKNL | KRAB (AA) |
| | VSLGYQLTKPDVILRLEKGEEPWLVEREIHQETHPDSETA | |
| | FEIKSSV | |
| 236 | ASPKKKRKVEASGSGMNIQMLLEAADYLERREREAEHGYA | SID4X domain |
| | SMLPGSGMNIQMLLEAADYLERREREAEHGYASMLPGSGM | (AA) |
| | NIQMLLEAADYLERREREAEHGYASMLPGSGMNIQMLLEA | |
| | ADYLERREREAEHGYASMLPSRSR | |
| 237 | MAAAVRMNIQMLLEAADYLERREREAEHGYASMLPYNNKD | MAD-SID domain |
| | RDALKRRNKSKKNNSSSRSTHNEMEKNRRAHLRLCLEKLK | (AA) |
| | GLVPLGPESSRHTTLSLLTKAKLHIKKLEDCDRKAVHQID | |
| | QLQREQRHLKRQLEKLGIERIRMDSIGSTVSSERSDSDRE | |
| | EIDVDVESTDYLTGDLDWSSSSVSDSDERGSMQSLGSDEG | |
| | YSSTSIKRIKLQDSHKACLG | |
| 238 | NHDQEFDPPKVYPPVPAEKRKPIRVLSLFDGIATGLLVLK | DNMT3A - |
| | DLGIQVDRYIASEVCEDSITVGMVRHQGKIMYVGDVRSVT | Human (AA) |
| | QKHIQEWGPFDLVIGGSPCNDLSIVNPARKGLYEGTGRLF | |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACV | |
| 239 | MKGDTRHLNGEEDAGGREDSILVNGACSDQSSDSPPILEA | DNMT3B (AA) |
| | IRTPEIRGRRSSSRLSKREVSSLLSYTQDLTGDGDGEDGD | |
| | GSDTPVMPKLFRETRTRSESPAVRTRNNNSVSSRERHRPS | |
| | PRSTRGRQGRNHVDESPVEFPATRSLRRRATASAGTPWPS | |
| | PPSSYLTIDLTDDTEDTHGTPQSSSTPYARLAQDSQQGGM | |
| | ESPQVEADSGDGDSSEYQDGKEFGIGDLVWGKIKGFSWWP | |
| | AMVVSWKATSKRQAMSGMRWVQWFGDGKFSEVSADKLVAL | |
| | GLFSQHFNLATENKLVSYRKAMYHALEKARVRAGKTFPSS | |
| | PGDSLEDQLKPMLEWAHGGFKPTGIEGLKPNNTQPVVNKS | |
| | KVRRAGSRKLESRKYENKTRRRTADDSATSDYCPAPKRLK | |
| | TNCYNNGKDRGDEDQSREQMASDVANNKSSLEDGCLSCGR | |
| | KNPVSFHPLFEGGLCQTCRDRFLELFYMYDDDGYQSYCTV | |
| | CCEGRELLLCSNTSCCRCFCVECLEVLVGTGTAAEAKLQE | |
| | PWSCYMCLPQRCHGVLRRRKDWNVRLQAFFTSDTGLEYEA | |
| | PKLYPAIPAARRRPIRVLSLFDGIATGYLVLKELGIKVGK | |
| | YVASEVCEESIAVGTVKHEGNIKYVNDVRNITKKNIEEWG | |
| | PFDLVIGGSPCNDLSNVNPARKGLYEGTGRLFFEFYHLLN | |
| | YSRPKEGDDRPFFWMFENVVAMKVGDKRDISRFLECNPVM | |
| | IDAIKVSAAHRARYFWGNLPGMNRPVIASKNDKLELQDCL | |
| | EYNRIAKLKKVQTITTKSNSIKQGKNQLFPVVMNGKEDVL | |
| | WCTELERIFGFPVHYTDVSNMGRGARQKLLGRSWSVPVIR | |
| | HLFAPLKDYFACE | |
| 240 | MGSRETPSSCSKTLETLDLETSDSSSPDADSPLEEQWLKS | DNMT3L - Murine |
| | SPALKEDSVDVVLEDCKEPLSPSSPPTGREMIRYEVKVNR | (AA) |
| | RSIEDICLCCGTLQVYTRHPLFEGGLCAPCKDKFLESLFL | |
| | YDDDGHQSYCTICCSGGTLFICESPDCTRCYCFECVDILV | |
| | GPGTSERINAMACWVCFLCLPFSRSGLLQRRKRWRHQLKA | |
| | FHDQEGAGPMEIYKTVSAWKRQPVRVLSLFRNIDKVLKSL | |
| | GFLESGSGSGGGTLKYVEDVTNVVRRDVEKWGPFDLVYGS | |
| | TQPLGSSCDRCPGWYMFQFHRILQYALPRQESQRPFFWIF | |
| | MDNLLLTEDDQETTTRFLQTEAVTLQDVRGRDYQNAMRVW | |
| | SNIPGLKSKHAPLTPKEEEYLQAQVRSRSKLDAPKVDLLV | |
| | KNCLLPLREYFKYFSQNSLPL | |
| 241 | MGPMEIYKTVSAWKRQPVRVLSLFRNIDKVLKSLGFLESG | DNMT3L - Murine |
| | SGSGGGTLKYVEDVTNVVRRDVEKWGPFDLVYGSTQPLGS | (AA) |
| | SCDRCPGWYMFQFHRILQYALPRQESQRPFFWIFMDNLLL | |
| | TEDDQETTTRFLQTEAVTLQDVRGRDYQNAMRVWSNIPGL | |
| | KSKHAPLTPKEEEYLQAQVRSRSKLDAPKVDLLVKNCLLP | |
| | LREYFKYFSQNSLPL | |
| 242 | MNPLEMFETVPVWRRQPVRVLSLFEDIKKELTSLGFLESG | C-terminal human |
| | SDPGQLKHVVDVTDTVRKDVEEWGPFDLVYGATPPLGHTC | DNMT3L (AA) |
| | DRPPSWYLFQFHRLLQYARPKPGSPRPFFWMFVDNLVLNK | |
| | EDLDVASRFLEMEPVTIPDVHGGSLQNAVRVWSNIPAIRS | |
| | RHWALVSEEELSLLAQNKQSSKLAAKWPTKLVKNCFLPLR | |
| | EYFKYFSTELTSSL | |
| 243 | SSGNSNANSRGPSFSSGLVPLSLRGSH | Linker (AA) |
| 244 | MLSGKKAAAAAAAAAAAATGTEAGPGTAGGSENGSEVAAQ | LSD1 (AA) |
| | PAGLSGPAEVGPGAVGERTPRKKEPPRASPPGGLAEPPGS | |
| | AGPQAGPTVVPGSATPMETGIAETPEGRRTSRRKRAKVEY | |
| | REMDESLANLSEDEYYSEEERNAKAEKEKKLPPPPPQAPP | |
| | EEENESEPEEPSGVEGAAFQSRLPHDRMTSQEAACFPDII | |
| | SGPQQTQKVFLFIRNRTLQLWLDNPKIQLTFEATLQQLEA | |
| | PYNSDTVLVHRVHSYLERHGLINFGIYKRIKPLPTKKTGK | |
| | VIIIGSGVSGLAAARQLQSFGMDVTLLEARDRVGGRVATF | |
| | RKGNYVADLGAMVVTGLGGNPMAVVSKQVNMELAKIKQKC | |
| | PLYEANGQAVPKEKDEMVEQEFNRLLEATSYLSHQLDFNV | |
| | LNNKPVSLGQALEVVIQLQEKHVKDEQIEHWKKIVKTQEE | |
| | LKELLNKMVNLKEKIKELHQQYKEASEVKPPRDITAEFLV | |
| | KSKHRDLTALCKEYDELAETQGKLEEKLQELEANPPSDVY | |
| | LSSRDRQILDWHFANLEFANATPLSTLSLKHWDQDDDFEF | |
| | TGSHLTVRNGYSCVPVALAEGLDIKLNTAVRQVRYTASGC | |
| | EVIAVNTRSTSQTFIYKCDAVLCTLPLGVLKQQPPAVQFV | |
| | PPLPEWKTSAVQRMGFGNLNKVVLCFDRVFWDPSVNLFGH | |
| | VGSTTASRGELFLFWNLYKAPILLALVAGEAAGIMENISD | |
| | DVIVGRCLAILKGIFGSSAVPQPKETVVSRWRADPWARGS | |
| | YSYVAAGSSGNDYDLMAQPITPGPSIPGAPQPIPRLFFAG | |
| | EHTIRNYPATVHGALLSGLREAGRIADQFLGAMYTLPRQA | |
| | TPGVPAQQSPSM | |
| 245 | MGQTGKKSEKGPVCWRKRVKSEYMRLRQLKRFRRADEVKT | EZH2 (AA) |
| | MFSSNRQKILERTETLNQEWKQRRIQPVHIMTSVSSLRGT | |
| | RECSVTSDLDFPAQVIPLKTLNAVASVPIMYSWSPLQQNF | |
| | MVEDETVLHNIPYMGDEVLDQDGTFIEELIKNYDGKVHGD | |
| | RECGFINDEIFVELVNALGQYNDDDDDDDGDDPDEREEKQ | |
| | KDLEDNRDDKETCPPRKFPADKIFEAISSMFPDKGTAEEL | |
| | KEKYKELTEQQLPGALPPECTPNIDGPNAKSVQREQSLHS | |
| | FHTLFCRRCFKYDCFLHPFHATPNTYKRKNTETALDNKPC | |
| | GPQCYQHLEGAKEFAAALTAERIKTPPKRPGGRRRGRLPN | |
| | NSSRPSTPTISVLESKDTDSDREAGTETGGENNDKEEEEK | |
| | KDETSSSSEANSRCQTPIKMKPNIEPPENVEWSGAEASMF | |
| | RVLIGTYYDNFCAIARLIGTKTCRQVYEFRVKESSIIAPV | |
| | PTEDVDTPPRKKKRKHRLWAAHCRKIQLKKDGSSNHVYNY | |
| | QPCDHPRQPCDSSCPCVIAQNFCEKFCQCSSECQNRFPGC | |
| | RCKAQCNTKQCPCYLAVRECDPDLCLTCGAADHWDSKNVS | |
| | CKNCSIQRGSKKHLLLAPSDVAGWGIFIKDPVQKNEFISE | |
| | YCGEIISQDEADRRGKVYDKYMCSFLFNLNNDFVVDATRK | |
| | GNKIRFANHSVNPNCYAKVMMVNGDHRIGIFAKRAIQTGE | |
| | ELFFDYRYSQADALKYVGIEREMEIP | |
| 246 | LLPKNYHLENEVARLKKLVGER | SunTag GCN4 |
| | | peptide (AA) |
| 247 | GGSGG | GGSGG linker |
| | | (AA) |
| 248 | PTQAGEGTLSEALLQLQFDDEDLGALLGNSTDPAVFTDLA | p65AD (aa) |
| | SVDNSEFQQLLNQGIPVAPHTTEPMLMEYPEAITRLVTGA | |
| | QRPPDPAPAPLGAPGLPNGLLSGDEDFSSIADMDFSALLS | |
| | QISS | |
| 249 | RDSREGMFLPKPEAGSAISDVFEGREVCQPKRIRPFHPPG | Rta domain (aa) |
| | SPWANRPLPASLAPTPTGPVHEPVGSLTPAPVPQPLDPAP | |
| | AVTPEASHLLEDPDEETSQAVKALREMADTVIPQKEEAAI | |
| | CGQMDLSHPPPRGHLDELTTTLESMTEDLNLDSPLTPELN | |
| | EILDTFLNDECLLHAMHISTGLSIFDTSLF | |
| 250 | MAENLLDGPPNPKRAKLSSPGFSANDSTDFGSLFDLENDL | CREB binding |
| | PDELIPNGGELGLLNSGNLVPDAASKHKQLSELLRGGSGS | protein (CBP) |
| | SINPGIGNVSASSPVQQGLGGQAQGQPNSANMASLSAMGK | (AA) |
| | SPLSQGDSSAPSLPKQAASTSGPTPAASQALNPQAQKQVG | |
| | LATSSPATSQTGPGICMNANFNQTHPGLLNSNSGHSLINQ | |
| | ASQGQAQVMNGSLGAAGRGRGAGMPYPTPAMQGASSSVLA | |
| | ETLTQVSPQMTGHAGLNTAQAGGMAKMGITGNTSPFGQPF | |
| | SQAGGQPMGATGVNPQLASKQSMVNSLPTFPTDIKNTSVT | |
| | NVPNMSQMQTSVGIVPTQAIATGPTADPEKRKLIQQQLVL | |
| | LLHAHKCQRREQANGEVRACSLPHCRTMKNVLNHMTHCQA | |
| | GKACQAILGSPASGIQNTIGSVGTGQQNATSLSNPNPIDP | |
| | SSMQRAYAALGLPYMNQPQTQLQPQVPGQQPAQPQTHQQM | |
| | RTLNPLGNNPMNIPAGGITTDQQPPNLISESALPTSLGAT | |
| | NPLMNDGSNSGNIGTLSTIPTAAPPSSTGVRKGWHEHVTQ | |
| | DLRSHLVHKLVQAIFPTPDPAALKDRRMENLVAYAKKVEG | |
| | DMYESANSRDEYYHLLAEKIYKIQKELEEKRRSRLHKQGI | |
| | LGNQPALPAPGAQPPVIPQAQPVRPPNGPLSLPVNRMQVS | |
| | QGMNSFNPMSLGNVQLPQAPMGPRAASPMNHSVQMNSMGS | |
| | VPGMAISPSRMPQPPNMMGAHTNNMMAQAPAQSQFLPQNQ | |
| | FPSSSGAMSVGMGQPPAQTGVSQGQVPGAALPNPLNMLGP | |
| | QASQLPCPPVTQSPLHPTPPPASTAAGMPSLQHTTPPGMT | |
| | PPQPAAPTQPSTPVSSSGQTPTPTPGSVPSATQTQSTPTV | |
| | QAAAQAQVTPQPQTPVQPPSVATPQSSQQQPTPVHAQPPG | |
| | TPLSQAAASIDNRVPTPSSVASAETNSQQPGPDVPVLEMK | |
| | TETQAEDTEPDPGESKGEPRSEMMEEDLQGASQVKEETDI | |
| | AEQKSEPMEVDEKKPEVKVEVKEEEESSSNGTASQSTSPS | |
| | QPRKKIFKPEELRQALMPTLEALYRQDPESLPFRQPVDPQ | |
| | LLGIPDYFDIVKNPMDLSTIKRKLDTGQYQEPWQYVDDVW | |
| | LMENNAWLYNRKTSRVYKFCSKLAEVFEQEIDPVMQSLGY | |
| | CCGRKYEFSPQTLCCYGKQLCTIPRDAAYYSYQNRYHFCE | |
| | KCFTEIQGENVTLGDDPSQPQTTISKDQFEKKKNDTLDPE | |
| | PFVDCKECGRKMHQICVLHYDIIWPSGFVCDNCLKKTGRP | |
| | RKENKFSAKRLQTTRLGNHLEDRVNKFLRRQNHPEAGEVF | |
| | VRVVASSDKTVEVKPGMKSRFVDSGEMSESFPYRTKALFA | |
| | FEEIDGVDVCFFGMHVQEYGSDCPPPNTRRVYISYLDSIH | |
| | FFRPRCLRTAVYHEILIGYLEYVKKLGYVTGHIWACPPSE | |
| | GDDYIFHCHPPDQKIPKPKRLQEWYKKMLDKAFAERIIHD | |
| | YKDIFKQATEDRLTSAKELPYFEGDFWPNVLEESIKELEQ | |
| | EEEERKKEESTAASETTEGSQGDSKNAKKKNNKKTNKNKS | |
| | SISRANKKKPSMPNVSNDLSQKLYATMEKHKEVFFVIHLH | |
| | AGPVINTLPPIVDPDPLLSCDLMDGRDAFLTLARDKHWEF | |
| | SSLRRSKWSTLCMLVELHTQGQDRFVYTCNECKHHVETRW | |
| | HCTVCEDYDLCINCYNTKSHAHKMVKWGLGLDDEGSSQGE | |
| | PQSKSPQESRRLSIQRCIQSLVHACQCRNANCSLPSCQKM | |
| | KRVVQHTKGCKRKTNGGCPVCKQLIALCCYHAKHCQENKC | |
| | PVPFCLNIKHKLRQQQIQHRLQQAQLMRRRMATMNTRNVP | |
| | QQSLPSPTSAPPGTPTQQPSTPQTPQPPAQPQPSPVSMSP | |
| | AGFPSVARTQPPTTVSTGKPTSQVPAPPPPAQPPPAAVEA | |
| | ARQIEREAQQQQHLYRVNINNSMPPGRTGMGTPGSQMAPV | |
| | SLNVPRPNQVSGPVMPSMPPGQWQQAPLPQQQPMPGLPRP | |
| | VISMQAQAAVAGPRMPSVQPPRSISPSALQDLLRTLKSPS | |
| | SPQQQQQVLNILKSNPQLMAAFIKQRTAKYVANQPGMQPQ | |
| | PGLQSQPGMQPQPGMHQQPSLQNLNAMQAGVPRPGVPPQQ | |
| | QAMGGLNPQGQALNIMNPGHNPNMASMNPQYREMLRRQLL | |
| | QQQQQQQQQQQQQQQQQQGSAGMAGGMAGHGQFQQPQGPG | |
| | GYPPAMQQQQRMQQHLPLQGSSMGQMAAQMGQLGQMGQPG | |
| | LGADSTPNIQQALQQRILQQQQMKQQIGSPGQPNPMSPQQ | |
| | HMLSGQPQASHLPGQQIATSLSNQVRSPAPVQSPRPQSQP | |
| | PHSSPSPRIQPQPSPHHVSPQTGSPHPGLAVTMASSIDQG | |
| | HLGNPEQSAMLPQLNTPSRSALSSELSLVGDTTGDTLEKF | |
| | VEGL | |
| 251 | MAENVVEPGPPSAKRPKLSSPALSASASDGTDFGSLFDLE | p300 (aa) |
| | HDLPDELINSTELGLTNGGDINQLQTSLGMVQDAASKHKQ | |
| | LSELLRSGSSPNLNMGVGGPGQVMASQAQQSSPGLGLINS | |
| | MVKSPMTQAGLTSPNMGMGTSGPNQGPTQSTGMMNSPVNQ | |
| | PAMGMNTGMNAGMNPGMLAAGNGQGIMPNQVMNGSIGAGR | |
| | GRQNMQYPNPGMGSAGNLLTEPLQQGSPQMGGQTGLRGPQ | |
| | PLKMGMMNNPNPYGSPYTQNPGQQIGASGLGLQIQTKTVL | |
| | SNNLSPFAMDKKAVPGGGMPNMGQQPAPQVQQPGLVTPVA | |
| | QGMGSGAHTADPEKRKLIQQQLVLLLHAHKCQRREQANGE | |
| | VRQCNLPHCRTMKNVLNHMTHCQSGKSCQVAHCASSRQII | |
| | SHWKNCTRHDCPVCLPLKNAGDKRNQQPILTGAPVGLGNP | |
| | SSLGVGQQSAPNLSTVSQIDPSSIERAYAALGLPYQVNQM | |
| | PTQPQVQAKNQQNQQPGQSPQGMRPMSNMSASPMGVNGGV | |
| | GVQTPSLLSDSMLHSAINSQNPMMSENASVPSMGPMPTAA | |
| | QPSTTGIRKQWHEDITQDLRNHLVHKLVQAIFPTPDPAAL | |
| | KDRRMENLVAYARKVEGDMYESANNRAEYYHLLAEKIYKI | |
| | QKELEEKRRTRLQKQNMLPNAAGMVPVSMNPGPNMGQPQP | |
| | GMTSNGPLPDPSMIRGSVPNQMMPRITPQSGLNQFGQMSM | |
| | AQPPIVPRQTPPLQHHGQLAQPGALNPPMGYGPRMQQPSN | |
| | QGQFLPQTQFPSQGMNVTNIPLAPSSGQAPVSQAQMSSSS | |
| | CPVNSPIMPPGSQGSHIHCPQLPQPALHQNSPSPVPSRTP | |
| | TPHHTPPSIGAQQPPATTIPAPVPTPPAMPPGPQSQALHP | |
| | PPRQTPTPPTTQLPQQVQPSLPAAPSADQPQQQPRSQQST | |
| | AASVPTPTAPLLPPQPATPLSQPAVSIEGQVSNPPSTSST | |
| | EVNSQAIAEKQPSQEVKMEAKMEVDQPEPADTQPEDISES | |
| | KVEDCKMESTETEERSTELKTEIKEEEDQPSTSATQSSPA | |
| | PGQSKKKIFKPEELRQALMPTLEALYRQDPESLPFRQPVD | |
| | PQLLGIPDYFDIVKSPMDLSTIKRKLDTGQYQEPWQYVDD | |
| | IWLMFNNAWLYNRKTSRVYKYCSKLSEVFEQEIDPVMQSL | |
| | GYCCGRKLEFSPQTLCCYGKQLCTIPRDATYYSYQNRYHF | |
| | CEKCFNEIQGESVSLGDDPSQPQTTINKEQFSKRKNDTLD | |
| | PELFVECTECGRKMHQICVLHHEIIWPAGFVCDGCLKKSA | |
| | RTRKENKFSAKRLPSTRLGTFLENRVNDFLRRQNHPESGE | |
| | VTVRVVHASDKTVEVKPGMKARFVDSGEMAESFPYRTKAL | |
| | FAFEEIDGVDLCFFGMHVQEYGSDCPPPNQRRVYISYLDS | |
| | VHFFRPKCLRTAVYHEILIGYLEYVKKLGYTTGHIWACPP | |
| | SEGDDYIFHCHPPDQKIPKPKRLQEWYKKMLDKAVSERIV | |
| | HDYKDIFKQATEDRLTSAKELPYFEGDFWPNVLEESIKEL | |
| | EQEEEERKREENTSNESTDVTKGDSKNAKKKNNKKTSKNK | |
| | SSLSRGNKKKPGMPNVSNDLSQKLYATMEKHKEVFFVIRL | |
| | IAGPAANSLPPIVDPDPLIPCDLMDGRDAFLTLARDKHLE | |
| | FSSLRRAQWSTMCMLVELHTQSQDRFVYTCNECKHHVETR | |
| | WHCTVCEDYDLCITCYNTKNHDHKMEKLGLGLDDESNNQQ | |
| | AAATQSPGDSRRLSIQRCIQSLVHACQCRNANCSLPSCQK | |
| | MKRVVQHTKGCKRKTNGGCPICKQLIALCCYHAKHCQENK | |
| | CPVPFCLNIKQKLRQQQLQHRLQQAQMLRRRMASMQRTGV | |
| | VGQQQGLPSPTPATPTTPTGQQPTTPQTPQPTSQPQPTPP | |
| | NSMPPYLPRTQAAGPVSQGKAAGQVTPPTPPQTAQPPLPG | |
| | PPPAAVEMAMQIQRAAETQRQMAHVQIFQRPIQHQMPPMT | |
| | PMAPMGMNPPPMTRGPSGHLEPGMGPTGMQQQPPWSQGGL | |
| | PQPQQLQSGMPRPAMMSVAQHGQPLNMAPQPGLGQVGISP | |
| | LKPGTVSQQALQNLLRTLRSPSSPLQQQQVLSILHANPQL | |
| | LAAFIKQRAAKYANSNPQPIPGQPGMPQGQPGLQPPTMPG | |
| | QQGVHSNPAMQNMNPMQAGVQRAGLPQQQPQQQLQPPMGG | |
| | MSPQAQQMNMNHNTMPSQFRDILRRQQMMQQQQQQGAGPG | |
| | IGPGMANHNQFQQPQGVGYPPQQQQRMQHHMQQMQQGNMG | |
| | QIGQLPQALGAEAGASLQAYQQRLLQQQMGSPVQPNPMSP | |
| | QQHMLPNQAQSPHLQGQQIPNSLSNQVRSPQPVPSPRPQS | |
| | QPPHSSPSPRMQPQPSPHHVSPQTSSPHPGLVAAQANPME | |
| | QGHFASPDQNSMLSQLASNPGMANLHGASATDLGLSTDNS | |
| | DLNSNLSQSTLDIH | |
| 252 | IFKPEELRQALMPTLEALYRQDPESLPFRQPVDPQLLGIP | p300 core (aa) |
| | DYFDIVKSPMDLSTIKRKLDTGQYQEPWQYVDDIWLMFNN | |
| | AWLYNRKTSRVYKYCSKLSEVFEQEIDPVMQSLGYCCGRK | |
| | LEFSPQTLCCYGKQLCTIPRDATYYSYQNRYHFCEKCFNE | |
| | IQGESVSLGDDPSQPQTTINKEQFSKRKNDTLDPELFVEC | |
| | TECGRKMHQICVLHHEIIWPAGFVCDGCLKKSARTRKENK | |
| | FSAKRLPSTRLGTFLENRVNDFLRRQNHPESGEVTVRVVH | |
| | ASDKTVEVKPGMKARFVDSGEMAESFPYRTKALFAFEEID | |
| | GVDLCFFGMHVQEYGSDCPPPNQRRVYISYLDSVHFFRPK | |
| | CLRTAVYHEILIGYLEYVKKLGYTTGHIWACPPSEGDDYI | |
| | FHCHPPDQKIPKPKRLQEWYKKMLDKAVSERIVHDYKDIF | |
| | KQATEDRLTSAKELPYFEGDFWPNVLEESIKELEQEEEER | |
| | KREENTSNESTDVTKGDSKNAKKKNNKKTSKNKSSLSRGN | |
| | KKKPGMPNVSNDLSQKLYATMEKHKEVFFVIRLIAGPAAN | |
| | SLPPIVDPDPLIPCDLMDGRDAFLTLARDKHLEFSSLRRA | |
| | QWSTMCMLVELHTQSQD | |
| 253 | GFSVDTSALLDLFSPSVTVPDMSLPDLDSSLASIQELLSP | HSF1 domain (aa) |
| | QEPPRPPEAENSSPDSGKQLVHYTAQPLFLLDPGSVDTGS | |
| | NDLPVLFELGEGSYFSEGDGFAEDPTISLLTGSEPPKAKD | |
| | PTVS | |
| 254 | MDLPVGPGAAGPSNVPAFLTKLWTLVSDPDTDALICWSPS | HSF1 (AA) |
| | GNSFHVFDQGQFAKEVLPKYFKHNNMASFVRQLNMYGFRK | |
| | VVHIEQGGLVKPERDDTEFQHPCFLRGQEQLLENIKRKVT | |
| | SVSTLKSEDIKIRQDSVTKLLTDVQLMKGKQECMDSKLLA | |
| | MKHENEALWREVASLRQKHAQQQKVVNKLIQFLISLVQSN | |
| | RILGVKRKIPLMLNDSGSAHSMPKYSRQFSLEHVHGSGPY | |
| | SAPSPAYSSSSLYAPDAVASSGPIISDITELAPASPMASP | |
| | GGSIDERPLSSSPLVRVKEEPPSPPQSPRVEEASPGRPSS | |
| | VDTLLSPTALIDSILRESEPAPASVTALTDARGHTDTEGR | |
| | PPSPPPTSTPEKCLSVACLDKNELSDHLDAMDSNLDNLQT | |
| | MLSSHGFSVDTSALLDLFSPSVTVPDMSLPDLDSSLASIQ | |
| | ELLSPQEPPRPPEAENSSPDSAGALHSAAAVPAGPRLRGH | |
| | REQRPAGAV | |
| 255 | DALDDFDLDMLGSDALDDFDLDMLGSDALDDFDLDMLGSD | VPR (aa) |
| | ALDDFDLDMLGSPKKKRKVGSQYLPDTDDRHRIEEKRKRT | |
| | YETFKSIMKKSPFSGPTDPRPPPRRIAVPSRSSASVPKPA | |
| | PQPYPFTSSLSTINYDEFPTMVFPSGQISQASALAPAPPQ | |
| | VLPQAPAPAPAPAMVSALAQAPAPVPVLAPGPPQAVAPPA | |
| | PKPTQAGEGTLSEALLQLQFDDEDLGALLGNSTDPAVFTD | |
| | LASVDNSEFQQLLNQGIPVAPHTTEPMLMEYPEAITRLVT | |
| | GAQRPPDPAPAPLGAPGLPNGLLSGDEDFSSIADMDFSAL | |
| | LSQISSGSGSGSRDSREGMFLPKPEAGSAISDVFEGREVC | |
| | QPKRIRPFHPPGSPWANRPLPASLAPTPTGPVHEPVGSLT | |
| | PAPVPQPLDPAPAVTPEASHLLEDPDEETSQAVKALREMA | |
| | DTVIPQKEEAAICGQMDLSHPPPRGHLDELTTTLESMTED | |
| | LNLDSPLTPELNEILDTFLNDECLLHAMHISTGLSIFDTS | |
| | LF | |
| 256 | DALDDFDLDMLGSDALDDFDLDMLGSDALDDFDLDMLGSD | VPH (aa) |
| | ALDDFDLDMLGSLPSASVEFEGSGGPSGQISNQALALAPS | |
| | SAPVLAQTMVPSSAMVPLAQPPAPAPVLTPGPPQSLSAPV | |
| | PKSTQAGEGTLSEALLHLQFDADEDLGALLGNSTDPGVFT | |
| | DLASVDNSEFQQLLNQGVSMSHSTAEPMLMEYPEAITRLV | |
| | TGSQRPPDPAPTPLGTSGLPNGLSGDEDFSSIADMDFSAL | |
| | LSQISSSGQGGGGSGFSVDTSALLDLFSPSVTVPDMSLPD | |
| | LDSSLASIQELLSPQEPPRPPEAENSSPDSGKQLVHYTAQ | |
| | PLFLLDPGSVDTGSNDLPVLFELGEGSYFSEGDGFAEDPT | |
| | ISLLTGSEPPKAKDPTVS | |
| 257 | MSRSRHARPSRLVRKEDVNKKKKNSQLRKTTKGANKNVAS | TET1 (AA) |
| | VKTLSPGKLKQLIQERDVKKKTEPKPPVPVRSLLTRAGAA | |
| | RMNLDRTEVLFQNPESLTCNGFTMALRSTSLSRRLSQPPL | |
| | VVAKSKKVPLSKGLEKQHDCDYKILPALGVKHSENDSVPM | |
| | QDTQVLPDIETLIGVQNPSLLKGKSQETTQFWSQRVEDSK | |
| | INIPTHSGPAAEILPGPLEGTRCGEGLFSEETLNDTSGSP | |
| | KMFAQDTVCAPFPQRATPKVTSQGNPSIQLEELGSRVESL | |
| | KLSDSYLDPIKSEHDCYPTSSLNKVIPDLNLRNCLALGGS | |
| | TSPTSVIKFLLAGSKQATLGAKPDHQEAFEATANQQEVSD | |
| | TTSFLGQAFGAIPHQWELPGADPVHGEALGETPDLPEIPG | |
| | AIPVQGEVFGTILDQQETLGMSGSVVPDLPVFLPVPPNPI | |
| | ATFNAPSKWPEPQSTVSYGLAVQGAIQILPLGSGHTPQSS | |
| | SNSEKNSLPPVMAISNVENEKQVHISFLPANTQGFPLAPE | |
| | RGLFHASLGIAQLSQAGPSKSDRGSSQVSVTSTVHVVNTT | |
| | VVTMPVPMVSTSSSSYTTLLPTLEKKKRKRCGVCEPCQQK | |
| | TNCGECTYCKNRKNSHQICKKRKCEELKKKPSVVVPLEVI | |
| | KENKRPQREKKPKVLKADFDNKPVNGPKSESMDYSRCGHG | |
| | EEQKLELNPHTVENVTKNEDSMTGIEVEKWTQNKKSQLTD | |
| | HVKGDFSANVPEAEKSKNSEVDKKRTKSPKLFVQTVRNGI | |
| | KHVHCLPAETNVSFKKFNIEEFGKTLENNSYKFLKDTANH | |
| | KNAMSSVATDMSCDHLKGRSNVLVFQQPGFNCSSIPHSSH | |
| | SIINHHASIHNEGDQPKTPENIPSKEPKDGSPVQPSLLSL | |
| | MKDRRLTLEQVVAIEALTQLSEAPSENSSPSKSEKDEESE | |
| | QRTASLLNSCKAILYTVRKDLQDPNLQGEPPKLNHCPSLE | |
| | KQSSCNTVVFNGQTTTLSNSHINSATNQASTKSHEYSKVT | |
| | NSLSLFIPKSNSSKIDTNKSIAQGIITLDNCSNDLHQLPP | |
| | RNNEVEYCNQLLDSSKKLDSDDLSCQDATHTQIEEDVATQ | |
| | LTQLASIIKINYIKPEDKKVESTPTSLVTCNVQQKYNQEK | |
| | GTIQQKPPSSVHNNHGSSLTKQKNPTQKKTKSTPSRDRRK | |
| | KKPTVVSYQENDRQKWEKLSYMYGTICDIWIASKFQNFGQ | |
| | FCPHDFPTVFGKISSSTKIWKPLAQTRSIMQPKTVFPPLT | |
| | QIKLQRYPESAEEKVKVEPLDSLSLFHLKTESNGKAFTDK | |
| | AYNSQVQLTVNANQKAHPLTQPSSPPNQCANVMAGDDQIR | |
| | FQQVVKEQLMHQRLPTLPGISHETPLPESALTLRNVNVVC | |
| | SGGITVVSTKSEEEVCSSSFGTSEFSTVDSAQKNFNDYAM | |
| | NFFTNPTKNLVSITKDSELPTCSCLDRVIQKDKGPYYTHL | |
| | GAGPSVAAVREIMENRYGQKGNAIRIEIVVYTGKEGKSSH | |
| | GCPIAKWVLRRSSDEEKVLCLVRQRTGHHCPTAVMVVLIM | |
| | VWDGIPLPMADRLYTELTENLKSYNGHPTDRRCTLNENRT | |
| | CTCQGIDPETCGASFSFGCSWSMYFNGCKFGRSPSPRRFR | |
| | IDPSSPLHEKNLEDNLQSLATRLAPIYKQYAPVAYQNQVE | |
| | YENVARECRLGSKEGRPFSGVTACLDFCAHPHRDIHNMNN | |
| | GSTVVCTLTREDNRSLGVIPQDEQLHVLPLYKLSDTDEFG | |
| | SKEGMEAKIKSGAIEVLAPRRKKRTCFTQPVPRSGKKRAA | |
| | MMTEVLAHKIRAVEKKPIPRIKRKNNSTTTNNSKPSSLPT | |
| | LGSNTETVQPEVKSETEPHFILKSSDNTKTYSLMPSAPHP | |
| | VKEASPGFSWSPKTASATPAPLKNDATASCGFSERSSTPH | |
| | CTMPSGRLSGANAAAADGPGISQLGEVAPLPTLSAPVMEP | |
| | LINSEPSTGVTEPLTPHQPNHQPSFLTSPQDLASSPMEED | |
| | EQHSEADEPPSDEPLSDDPLSPAEEKLPHIDEYWSDSEHI | |
| | FLDANIGGVAIAPAHGSVLIECARRELHATTPVEHPNRNH | |
| | PTRLSLVFYQHKNLNKPQHGFELNKIKFEAKEAKNKKMKA | |
| | SEQKDQAANEGPEQSSEVNELNQIPSHKALTLTHDNVVTV | |
| | SPYALTHVAGPYNHWV | |
| 258 | LPTCSCLDRVIQKDKGPYYTHLGAGPSVAAVREIMENRYG | TET1 catalytic |
| | QKGNAIRIEIVVYTGKEGKSSHGCPIAKWVLRRSSDEEKV | domain (aa) |
| | LCLVRQRTGHHCPTAVMVVLIMVWDGIPLPMADRLYTELT | |
| | ENLKSYNGHPTDRRCTLNENRTCTCQGIDPETCGASFSFG | |
| | CSWSMYFNGCKFGRSPSPRRFRIDPSSPLHEKNLEDNLQS | |
| | LATRLAPIYKQYAPVAYQNQVEYENVARECRLGSKEGRPF | |
| | SGVTACLDFCAHPHRDIHNMNNGSTVVCTLTREDNRSLGV | |
| | IPQDEQLHVLPLYKLSDTDEFGSKEGMEAKIKSGAIEVLA | |
| | PRRKKRTCFTQPVPRSGKKRAAMMTEVLAHKIRAVEKKPI | |
| | PRIKRKNNSTTTNNSKPSSLPTLGSNTETVQPEVKSETEP | |
| | HFILKSSDNTKTYSLMPSAPHPVKEASPGFSWSPKTASAT | |
| | PAPLKNDATASCGFSERSSTPHCTMPSGRLSGANAAAADG | |
| | PGISQLGEVAPLPTLSAPVMEPLINSEPSTGVTEPLTPHQ | |
| | PNHQPSFLTSPQDLASSPMEEDEQHSEADEPPSDEPLSDD | |
| | PLSPAEEKLPHIDEYWSDSEHIFLDANIGGVAIAPAHGSV | |
| | LIECARRELHATTPVEHPNRNHPTRLSLVFYQHKNLNKPQ | |
| | HGFELNKIKFEAKEAKNKKMKASEQKDQAANEGPEQSSEV | |
| | NELNQIPSHKALTLTHDNVVTVSPYALTHVAGPYNHWV | |
| 259 | GGGGS | Linker (AA) |
| 260 | GGGGG | Linker (AA) |
| 261 | GGAGG | Linker (AA) |
| 262 | GGGGSSS | Linker (AA) |
| 263 | GGGGAAA | Linker (AA) |
| 264 | EASGSGRASPGIPGSTR | Linker (AA) |
| 265 | GIHGVPAA | Linker (AA) |
| 266 | KRPAATKKAGQAKKKKASDAKSLTAWS | Linker (AA) |
| 267 | GGPSSGAPPPSGGSPAGSPTSTEEGTSESATPESGPGTST | XTEN80 (aa) |
| | EPSEGSAPGSPAGSPTSTEEGTSTEPSEGSAPGTSTEPSE | |
| 268 | SGSETPGTSESATPES | Linker (AA) |
| 269 | PKKKRKV | NLS |
| 270 | KRPAATKKAGQAKKKK | NLS |
| 271 | PAAKRVKLD | NLS |
| 272 | RQRRNELKRSP | NLS |
| 273 | NQSSNFGPMKGGNFGGRSSGPYGGGGQYFAKPRNQGGY | NLS |
| 274 | RMRIZFKNKGKDTAELRRRRVEVSVELRKAKKDEQILKRR | NLS |
| | NV | |
| 275 | VSRKRPRP | NLS |
| 276 | PPKKARED | NLS |
| 277 | PQPKKKPL | NLS |
| 278 | SALIKKKKKMAP | NLS |
| 279 | DRLRR | NLS |
| 280 | PKQKKRK | NLS |
| 281 | RKLKKKIKKL | NLS |
| 282 | REKKKFLKRR | NLS |
| 283 | KRKGDEVDGVDEVAKKKSKK | NLS |
| 284 | RKCLQAGMNLEARKTKK | NLS |
| 285 | PKKKRKVGIHGVPAA | Linker (AA) |
| 286 | DYKDDDDK | FLAG tag (AA) |
| 287 | MDYKDHDGDYKDHDIDYKDDDDK | 3x FLAG tag (AA) |
| 288 | YPYDVPDYA | HA tag (AA) |
| 289 | HHHHHH | His tag (AA) |
| 290 | CLSYETEILTVEYGLLPIGKIVEKRIECTVYSVDNNGNIY | N term Npu Intein |
| | TQPVAQWHDRGEQEVFEYCLEDGSLIRATKDHKFMTVDGQ | (aa) |
| | MLPID | |
| 291 | IKIATRKYLGKQNVYDIGVERDHNFALKNGFIASN | C term Npu Intein |
| | | (aa) |
| 292 | TTCCCCAACTCCGTACTGAG | FAS_7 target site |
| 293 | GGGAAGCTCTTTCACTTCGG | FAS_8 target site |
| 294 | ACTGTAAGTCGCTGCCTGAGTGG | FAS_9 target site |
| 295 | GAGCGGGTCCACCAACCCGC | FAS_10 target site |
| 296 | UUCCCCAACUCCGUACUGAG | FAS_7 gRNA |
| | | spacer |
| 297 | GGGAAGCUCUUUCACUUCGG | FAS_8 gRNA |
| | | spacer |
| 298 | ACUGUAAGUCGCUGCCUGAGUGG | FAS_9 gRNA |
| | | spacer |
| 299 | GAGCGGGUCCACCAACCCGC | FAS_10 gRNA |
| | | spacer |
| 300 | ACTTCAACTCAGCGCTGCGG | TGFBR2_1 target |
| | | site |
| 301 | AGTCCGGCTCCTGTCCCGAG | TGFBR2_2 target |
| | | site |
| 302 | GAAACTCCTCGCCAACAGCT | TGFBR2_3 target |
| | | site |
| 303 | ACUUCAACUCAGCGCUGCGG | TGFBR2_1 gRNA |
| | | spacer |
| 304 | AGUCCGGCUCCUGUCCCGAG | TGFBR2_2 gRNA |
| | | spacer |
| 305 | GAAACUCCUCGCCAACAGCU | TGFBR2_3 gRNA |
| | | spacer |
| 306 | GTCCCGAGCGGGTGCACGCG | TGFBR2_4 target |
| | | site |
| 307 | GTCCGGCTCCTGTCCCGAGC | TGFBR2_5 target |
| | | site |
| 308 | CCCGAGCGGGTGCACGCGCG | TGFBR2_6 target |
| | | site |
| 309 | GUCCCGAGCGGGUGCACGCG | TGFBR2_4 gRNA |
| | | spacer |
| 310 | GUCCGGCUCCUGUCCCGAGC | TGFBR2_5 gRNA |
| | | spacer |
| 311 | CCCGAGCGGGUGCACGCGCG | TGFBR2_6 gRNA |
| | | spacer |
| 312 | MDYKDHDGDYKDHDIDYKDDDDKHVNPKKKRKVGIHGVPA | dSpCas9-KOX1(2- |
| | ADKKYSIGLAIGTNSVGWAVITDEYKVPSKKFKVLGNTDR | 99) |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASDAKSLTAWSRT | |
| | LVTFKDVFVDFTREEWKLLDTAQQILYRNVMLENYKNLVS | |
| | LGYQLTKPDVILRLEKGEEPWLVEREIHQETHPDSETAFE | |
| | IKSSVPKKKRKVASATNFSLLKQAGDVEENPGPMVSKGEE | |
| | DNMAIIKEFMRFKVHMEGSVNGHEFEIEGEGEGRPYEGTQ | |
| | TAKLKVTKGGPLPFAWDILSPQFMYGSKAYVKHPADIPDY | |
| | LKLSFPEGFKWERVMNFEDGGVVTVTQDSSLQDGEFIYKV | |
| | KLRGTNFPSDGPVMQKKTMGWEASSERMYPEDGALKGEIK | |
| | QRLKLKDGGHYDAEVKTTYKAKKPVQLPGAYNVNIKLDIT | |
| | SHNEDYTIVEQYERAEGRHSTGGMDELYK | |
| 313 | MDYKDHDGDYKDHDIDYKDDDDKHVNPKKKRKVGIHGVPA | dSpCas9-KOX1(1- |
| | ADKKYSIGLAIGTNSVGWAVITDEYKVPSKKFKVLGNTDR | 72) |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASMDAKSLTAWSR | |
| | TLVTFKDVFVDFTREEWKLLDTAQQIVYRNVMLENYKNLV | |
| | SLGYQLTKPDVILRLEKGEEPPKKKRKVASATNFSLLKQA | |
| | GDVEENPGPMVSKGEEDNMAIIKEFMRFKVHMEGSVNGHE | |
| | FEIEGEGEGRPYEGTQTAKLKVTKGGPLPFAWDILSPQFM | |
| | YGSKAYVKHPADIPDYLKLSFPEGFKWERVMNFEDGGVVT | |
| | VTQDSSLQDGEFIYKVKLRGTNFPSDGPVMQKKTMGWEAS | |
| | SERMYPEDGALKGEIKQRLKLKDGGHYDAEVKTTYKAKKP | |
| | VQLPGAYNVNIKLDITSHNEDYTIVEQYERAEGRHSTGGM | |
| | DELYK | |
| 314 | MDYKDHDGDYKDHDIDYKDDDDKHVNPKKKRKVGIHGVPA | dSpCas9-ZIM3 |
| | ADKKYSIGLAIGTNSVGWAVITDEYKVPSKKFKVLGNTDR | |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASNNSQGRVTFED | |
| | VTVNFTQGEWQRLNPEQRNLYRDVMLENYSNLVSVGQGET | |
| | TKPDVILRLEQGKEPWLEEEEVLGSGRAEKNGDIGGQIWK | |
| | PKDVKESLPKKKRKVASATNFSLLKQAGDVEENPGPMVSK | |
| | GEEDNMAIIKEFMRFKVHMEGSVNGHEFEIEGEGEGRPYE | |
| | GTQTAKLKVTKGGPLPFAWDILSPQFMYGSKAYVKHPADI | |
| | PDYLKLSFPEGFKWERVMNFEDGGVVTVTQDSSLQDGEFI | |
| | YKVKLRGTNFPSDGPVMQKKTMGWEASSERMYPEDGALKG | |
| | EIKQRLKLKDGGHYDAEVKTTYKAKKPVQLPGAYNVNIKL | |
| | DITSHNEDYTIVEQYERAEGRHSTGGMDELYK | |
| 315 | MDYKDHDGDYKDHDIDYKDDDDKHVNPKKKRKVGIHGVPA | dSpCas9-ZNF324 |
| | ADKKYSIGLAIGTNSVGWAVITDEYKVPSKKFKVLGNTDR | |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASAFEDVAVYFSQ | |
| | EEWGLLDTAQRALYRRVMLDNFALVASLGLSTSRPRVVIQ | |
| | LERGEEPWVPSGTDTTLSRTTYRRRNPGSWSLTEDRDVSG | |
| | PKKKRKVASATNFSLLKQAGDVEENPGPMVSKGEEDNMAI | |
| | IKEFMRFKVHMEGSVNGHEFEIEGEGEGRPYEGTQTAKLK | |
| | VTKGGPLPFAWDILSPQFMYGSKAYVKHPADIPDYLKLSF | |
| | PEGFKWERVMNFEDGGVVTVTQDSSLQDGEFIYKVKLRGT | |
| | NFPSDGPVMQKKTMGWEASSERMYPEDGALKGEIKQRLKL | |
| | KDGGHYDAEVKTTYKAKKPVQLPGAYNVNIKLDITSHNED | |
| | YTIVEQYERAEGRHSTGGMDELYK | |
| 316 | MDYKDHDGDYKDHDIDYKDDDDKHVNPKKKRKVGIHGVPA | dSpCas9-EZH2 |
| | ADKKYSIGLAIGTNSVGWAVITDEYKVPSKKFKVLGNTDR | |
| | HSIKKNLIGALLFDSGETAEATRLKRTARRRYTRRKNRIC | |
| | YLQEIFSNEMAKVDDSFFHRLEESFLVEEDKKHERHPIFG | |
| | NIVDEVAYHEKYPTIYHLRKKLVDSTDKADLRLIYLALAH | |
| | MIKFRGHFLIEGDLNPDNSDVDKLFIQLVQTYNQLFEENP | |
| | INASGVDAKAILSARLSKSRRLENLIAQLPGEKKNGLFGN | |
| | LIALSLGLTPNFKSNFDLAEDAKLQLSKDTYDDDLDNLLA | |
| | QIGDQYADLFLAAKNLSDAILLSDILRVNTEITKAPLSAS | |
| | MIKRYDEHHQDLTLLKALVRQQLPEKYKEIFFDQSKNGYA | |
| | GYIDGGASQEEFYKFIKPILEKMDGTEELLVKLNREDLLR | |
| | KQRTFDNGSIPHQIHLGELHAILRRQEDFYPFLKDNREKI | |
| | EKILTFRIPYYVGPLARGNSRFAWMTRKSEETITPWNFEE | |
| | VVDKGASAQSFIERMTNFDKNLPNEKVLPKHSLLYEYFTV | |
| | YNELTKVKYVTEGMRKPAFLSGEQKKAIVDLLFKTNRKVT | |
| | VKQLKEDYFKKIECFDSVEISGVEDRFNASLGTYHDLLKI | |
| | IKDKDFLDNEENEDILEDIVLTLTLFEDREMIEERLKTYA | |
| | HLFDDKVMKQLKRRRYTGWGRLSRKLINGIRDKQSGKTIL | |
| | DFLKSDGFANRNFMQLIHDDSLTFKEDIQKAQVSGQGDSL | |
| | HEHIANLAGSPAIKKGILQTVKVVDELVKVMGRHKPENIV | |
| | IEMARENQTTQKGQKNSRERMKRIEEGIKELGSQILKEHP | |
| | VENTQLQNEKLYLYYLQNGRDMYVDQELDINRLSDYDVDA | |
| | IVPQSFLKDDSIDNKVLTRSDKNRGKSDNVPSEEVVKKMK | |
| | NYWRQLLNAKLITQRKFDNLTKAERGGLSELDKAGFIKRQ | |
| | LVETRQITKHVAQILDSRMNTKYDENDKLIREVKVITLKS | |
| | KLVSDFRKDFQFYKVREINNYHHAHDAYLNAVVGTALIKK | |
| | YPKLESEFVYGDYKVYDVRKMIAKSEQEIGKATAKYFFYS | |
| | NIMNFFKTEITLANGEIRKRPLIETNGETGEIVWDKGRDF | |
| | ATVRKVLSMPQVNIVKKTEVQTGGFSKESILPKRNSDKLI | |
| | ARKKDWDPKKYGGFDSPTVAYSVLVVAKVEKGKSKKLKSV | |
| | KELLGITIMERSSFEKNPIDFLEAKGYKEVKKDLIIKLPK | |
| | YSLFELENGRKRMLASAGELQKGNELALPSKYVNFLYLAS | |
| | HYEKLKGSPEDNEQKQLFVEQHKHYLDEIIEQISEFSKRV | |
| | ILADANLDKVLSAYNKHRDKPIREQAENIIHLFTLTNLGA | |
| | PAAFKYFDTTIDRKRYTSTKEVLDATLIHQSITGLYETRI | |
| | DLSQLGGDSGGKRPAATKKAGQAKKKKASSTGGSGGSGGS | |
| | GGSGGSGRPGQTGKKSEKGPVCWRKRVKSEYMRLRQLKRF | |
| | RRADEVKTMFSSNRQKILERTETLNQEWKQRRIQPVHIMT | |
| | SVSSLRGTRECSVTSDLDFPAQVIPLKTLNAVASVPIMYS | |
| | WSPLQQNFMVEDETVLHNIPYMGDEVLDQDGTFIEELIKN | |
| | YDGKVHGDRECGFINDEIFVELVNALGQYNDDDDDDDGDD | |
| | PDEREEKQKDLEDNRDDKETCPPRKFPADKIFEAISSMFP | |
| | DKGTAEELKEKYKELTEQQLPGALPPECTPNIDGPNAKSV | |
| | QREQSLHSFHTLFCRRCFKYDCFLHPFHATPNTYKRKNTE | |
| | TALDNKPCGPQCYQHLEGAKEFAAALTAERIKTPPKRPGG | |
| | RRRGRLPNNSSRPSTPTISVLESKDTDSDREAGTETGGEN | |
| | NDKEEEEKKDETSSSSEANSRCQTPIKMKPNIEPPENVEW | |
| | SGAEASMFRVLIGTYYDNFCAIARLIGTKTCRQVYEFRVK | |
| | ESSIIAPVPTEDVDTPPRKKKRKHRLWAAHCRKIQLKKDG | |
| | SSNHVYNYQPCDHPRQPCDSSCPCVIAQNFCEKFCQCSSE | |
| | CQNRFPGCRCKAQCNTKQCPCYLAVRECDPDLCLTCGAAD | |
| | HWDSKNVSCKNCSIQRGSKKHLLLAPSDVAGWGIFIKDPV | |
| | QKNEFISEYCGEIISQDEADRRGKVYDKYMCSFLFNLNND | |
| | FVVDATRKGNKIRFANHSVNPNCYAKVMMVNGDHRIGIFA | |
| | KRAIQTGEELFFDYRYSQADALKYVGIEREMEIPPKKKRK | |
| | VASATNFSLLKQAGDVEENPGPMVSKGEEDNMAIIKEFMR | |
| | FKVHMEGSVNGHEFEIEGEGEGRPYEGTQTAKLKVTKGGP | |
| | LPFAWDILSPQFMYGSKAYVKHPADIPDYLKLSFPEGFKW | |
| | ERVMNFEDGGVVTVTQDSSLQDGEFIYKVKLRGTNFPSDG | |
| | PVMQKKTMGWEASSERMYPEDGALKGEIKQRLKLKDGGHY | |
| | DAEVKTTYKAKKPVQLPGAYNVNIKLDITSHNEDYTIVEQ | |
| | YERAEGRHSTGGMDELYK | |
| 317 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | dSpCas9-KOX1(2- |
| | EDSITVGMVRHQGKIMYVGDVRSVTQKHIQEWGPFDLVIG | 99) |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSENPKKKRKVGIHGVPAADKKYSIGLAIGTNSVG | |
| | WAVITDEYKVPSKKFKVLGNTDRHSIKKNLIGALLFDSGE | |
| | TAEATRLKRTARRRYTRRKNRICYLQEIFSNEMAKVDDSF | |
| | FHRLEESFLVEEDKKHERHPIFGNIVDEVAYHEKYPTIYH | |
| | LRKKLVDSTDKADLRLIYLALAHMIKFRGHFLIEGDLNPD | |
| | NSDVDKLFIQLVQTYNQLFEENPINASGVDAKAILSARLS | |
| | KSRRLENLIAQLPGEKKNGLFGNLIALSLGLTPNFKSNFD | |
| | LAEDAKLQLSKDTYDDDLDNLLAQIGDQYADLFLAAKNLS | |
| | DAILLSDILRVNTEITKAPLSASMIKRYDEHHQDLTLLKA | |
| | LVRQQLPEKYKEIFFDQSKNGYAGYIDGGASQEEFYKFIK | |
| | PILEKMDGTEELLVKLNREDLLRKQRTFDNGSIPHQIHLG | |
| | ELHAILRRQEDFYPFLKDNREKIEKILTFRIPYYVGPLAR | |
| | GNSRFAWMTRKSEETITPWNFEEVVDKGASAQSFIERMTN | |
| | FDKNLPNEKVLPKHSLLYEYFTVYNELTKVKYVTEGMRKP | |
| | AFLSGEQKKAIVDLLFKTNRKVTVKQLKEDYFKKIECFDS | |
| | VEISGVEDRFNASLGTYHDLLKIIKDKDFLDNEENEDILE | |
| | DIVLTLTLFEDREMIEERLKTYAHLFDDKVMKQLKRRRYT | |
| | GWGRLSRKLINGIRDKQSGKTILDFLKSDGFANRNFMQLI | |
| | HDDSLTFKEDIQKAQVSGQGDSLHEHIANLAGSPAIKKGI | |
| | LQTVKVVDELVKVMGRHKPENIVIEMARENQTTQKGQKNS | |
| | RERMKRIEEGIKELGSQILKEHPVENTQLQNEKLYLYYLQ | |
| | NGRDMYVDQELDINRLSDYDVDAIVPQSFLKDDSIDNKVL | |
| | TRSDKNRGKSDNVPSEEVVKKMKNYWRQLLNAKLITQRKF | |
| | DNLTKAERGGLSELDKAGFIKRQLVETRQITKHVAQILDS | |
| | RMNTKYDENDKLIREVKVITLKSKLVSDFRKDFQFYKVRE | |
| | INNYHHAHDAYLNAVVGTALIKKYPKLESEFVYGDYKVYD | |
| | VRKMIAKSEQEIGKATAKYFFYSNIMNFFKTEITLANGEI | |
| | RKRPLIETNGETGEIVWDKGRDFATVRKVLSMPQVNIVKK | |
| | TEVQTGGFSKESILPKRNSDKLIARKKDWDPKKYGGFDSP | |
| | TVAYSVLVVAKVEKGKSKKLKSVKELLGITIMERSSFEKN | |
| | PIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYT | |
| | STKEVLDATLIHQSITGLYETRIDLSQLGGDSGGKRPAAT | |
| | KKAGQAKKKKASDAKSLTAWSRTLVTFKDVFVDFTREEWK | |
| | LLDTAQQILYRNVMLENYKNLVSLGYQLTKPDVILRLEKG | |
| | EEPWLVEREIHQETHPDSETAFEIKSSVPKKKRKVASATN | |
| | FSLLKQAGDVEENPGPMVSKGEEDNMAIIKEFMRFKVHME | |
| | GSVNGHEFEIEGEGEGRPYEGTQTAKLKVTKGGPLPFAWD | |
| | ILSPQFMYGSKAYVKHPADIPDYLKLSFPEGFKWERVMNF | |
| | EDGGVVTVTQDSSLQDGEFIYKVKLRGTNFPSDGPVMQKK | |
| | TMGWEASSERMYPEDGALKGEIKQRLKLKDGGHYDAEVKT | |
| | TYKAKKPVQLPGAYNVNIKLDITSHNEDYTIVEQYERAEG | |
| | RHSTGGMDELYK | |
| 318 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | dSpCas9-KOX1(1- |
| | EDSITVGMVRHQGKIMYVGDVRSVTQKHIQEWGPFDLVIG | 72) |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSENPKKKRKVGIHGVPAADKKYSIGLAIGTNSVG | |
| | WAVITDEYKVPSKKFKVLGNTDRHSIKKNLIGALLFDSGE | |
| | TAEATRLKRTARRRYTRRKNRICYLQEIFSNEMAKVDDSF | |
| | FHRLEESFLVEEDKKHERHPIFGNIVDEVAYHEKYPTIYH | |
| | LRKKLVDSTDKADLRLIYLALAHMIKFRGHFLIEGDLNPD | |
| | NSDVDKLFIQLVQTYNQLFEENPINASGVDAKAILSARLS | |
| | KSRRLENLIAQLPGEKKNGLFGNLIALSLGLTPNFKSNFD | |
| | LAEDAKLQLSKDTYDDDLDNLLAQIGDQYADLFLAAKNLS | |
| | DAILLSDILRVNTEITKAPLSASMIKRYDEHHQDLTLLKA | |
| | LVRQQLPEKYKEIFFDQSKNGYAGYIDGGASQEEFYKFIK | |
| | PILEKMDGTEELLVKLNREDLLRKQRTFDNGSIPHQIHLG | |
| | ELHAILRRQEDFYPFLKDNREKIEKILTFRIPYYVGPLAR | |
| | GNSRFAWMTRKSEETITPWNFEEVVDKGASAQSFIERMTN | |
| | FDKNLPNEKVLPKHSLLYEYFTVYNELTKVKYVTEGMRKP | |
| | AFLSGEQKKAIVDLLFKTNRKVTVKQLKEDYFKKIECFDS | |
| | VEISGVEDRFNASLGTYHDLLKIIKDKDFLDNEENEDILE | |
| | DIVLTLTLFEDREMIEERLKTYAHLFDDKVMKQLKRRRYT | |
| | GWGRLSRKLINGIRDKQSGKTILDFLKSDGFANRNFMQLI | |
| | HDDSLTFKEDIQKAQVSGQGDSLHEHIANLAGSPAIKKGI | |
| | LQTVKVVDELVKVMGRHKPENIVIEMARENQTTQKGQKNS | |
| | RERMKRIEEGIKELGSQILKEHPVENTQLQNEKLYLYYLQ | |
| | NGRDMYVDQELDINRLSDYDVDAIVPQSFLKDDSIDNKVL | |
| | TRSDKNRGKSDNVPSEEVVKKMKNYWRQLLNAKLITQRKF | |
| | DNLTKAERGGLSELDKAGFIKRQLVETRQITKHVAQILDS | |
| | RMNTKYDENDKLIREVKVITLKSKLVSDFRKDFQFYKVRE | |
| | INNYHHAHDAYLNAVVGTALIKKYPKLESEFVYGDYKVYD | |
| | VRKMIAKSEQEIGKATAKYFFYSNIMNFFKTEITLANGEI | |
| | RKRPLIETNGETGEIVWDKGRDFATVRKVLSMPQVNIVKK | |
| | TEVQTGGFSKESILPKRNSDKLIARKKDWDPKKYGGFDSP | |
| | TVAYSVLVVAKVEKGKSKKLKSVKELLGITIMERSSFEKN | |
| | PIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYT | |
| | STKEVLDATLIHQSITGLYETRIDLSQLGGDSGGKRPAAT | |
| | KKAGQAKKKKASMDAKSLTAWSRTLVTFKDVFVDFTREEW | |
| | KLLDTAQQIVYRNVMLENYKNLVSLGYQLTKPDVILRLEK | |
| | GEEPPKKKRKVASATNFSLLKQAGDVEENPGPMVSKGEED | |
| | NMAIIKEFMRFKVHMEGSVNGHEFEIEGEGEGRPYEGTQT | |
| | AKLKVTKGGPLPFAWDILSPQFMYGSKAYVKHPADIPDYL | |
| | KLSFPEGFKWERVMNFEDGGVVTVTQDSSLQDGEFIYKVK | |
| | LRGTNFPSDGPVMQKKTMGWEASSERMYPEDGALKGEIKQ | |
| | RLKLKDGGHYDAEVKTTYKAKKPVQLPGAYNVNIKLDITS | |
| | HNEDYTIVEQYERAEGRHSTGGMDELYK | |
| 319 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | dSpCas9-ZIM3 |
| | EDSITVGMVRHQGKIMYVGDVRSVTQKHIQEWGPFDLVIG | |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSENPKKKRKVGIHGVPAADKKYSIGLAIGTNSVG | |
| | WAVITDEYKVPSKKFKVLGNTDRHSIKKNLIGALLFDSGE | |
| | TAEATRLKRTARRRYTRRKNRICYLQEIFSNEMAKVDDSF | |
| | FHRLEESFLVEEDKKHERHPIFGNIVDEVAYHEKYPTIYH | |
| | LRKKLVDSTDKADLRLIYLALAHMIKFRGHFLIEGDLNPD | |
| | NSDVDKLFIQLVQTYNQLFEENPINASGVDAKAILSARLS | |
| | KSRRLENLIAQLPGEKKNGLFGNLIALSLGLTPNFKSNFD | |
| | LAEDAKLQLSKDTYDDDLDNLLAQIGDQYADLFLAAKNLS | |
| | DAILLSDILRVNTEITKAPLSASMIKRYDEHHQDLTLLKA | |
| | LVRQQLPEKYKEIFFDQSKNGYAGYIDGGASQEEFYKFIK | |
| | PILEKMDGTEELLVKLNREDLLRKQRTFDNGSIPHQIHLG | |
| | ELHAILRRQEDFYPFLKDNREKIEKILTFRIPYYVGPLAR | |
| | GNSRFAWMTRKSEETITPWNFEEVVDKGASAQSFIERMTN | |
| | FDKNLPNEKVLPKHSLLYEYFTVYNELTKVKYVTEGMRKP | |
| | AFLSGEQKKAIVDLLFKTNRKVTVKQLKEDYFKKIECFDS | |
| | VEISGVEDRFNASLGTYHDLLKIIKDKDFLDNEENEDILE | |
| | DIVLTLTLFEDREMIEERLKTYAHLFDDKVMKQLKRRRYT | |
| | GWGRLSRKLINGIRDKQSGKTILDFLKSDGFANRNFMQLI | |
| | HDDSLTFKEDIQKAQVSGQGDSLHEHIANLAGSPAIKKGI | |
| | LQTVKVVDELVKVMGRHKPENIVIEMARENQTTQKGQKNS | |
| | RERMKRIEEGIKELGSQILKEHPVENTQLQNEKLYLYYLQ | |
| | NGRDMYVDQELDINRLSDYDVDAIVPQSFLKDDSIDNKVL | |
| | TRSDKNRGKSDNVPSEEVVKKMKNYWRQLLNAKLITQRKF | |
| | DNLTKAERGGLSELDKAGFIKRQLVETRQITKHVAQILDS | |
| | RMNTKYDENDKLIREVKVITLKSKLVSDFRKDFQFYKVRE | |
| | INNYHHAHDAYLNAVVGTALIKKYPKLESEFVYGDYKVYD | |
| | VRKMIAKSEQEIGKATAKYFFYSNIMNFFKTEITLANGEI | |
| | RKRPLIETNGETGEIVWDKGRDFATVRKVLSMPQVNIVKK | |
| | TEVQTGGFSKESILPKRNSDKLIARKKDWDPKKYGGFDSP | |
| | TVAYSVLVVAKVEKGKSKKLKSVKELLGITIMERSSFEKN | |
| | PIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYT | |
| | STKEVLDATLIHQSITGLYETRIDLSQLGGDSGGKRPAAT | |
| | KKAGQAKKKKASNNSQGRVTFEDVTVNFTQGEWQRLNPEQ | |
| | RNLYRDVMLENYSNLVSVGQGETTKPDVILRLEQGKEPWL | |
| | EEEEVLGSGRAEKNGDIGGQIWKPKDVKESLPKKKRKVAS | |
| | ATNFSLLKQAGDVEENPGPMVSKGEEDNMAIIKEFMRFKV | |
| | HMEGSVNGHEFEIEGEGEGRPYEGTQTAKLKVTKGGPLPF | |
| | AWDILSPQFMYGSKAYVKHPADIPDYLKLSFPEGFKWERV | |
| | MNFEDGGVVTVTQDSSLQDGEFIYKVKLRGTNFPSDGPVM | |
| | QKKTMGWEASSERMYPEDGALKGEIKQRLKLKDGGHYDAE | |
| | VKTTYKAKKPVQLPGAYNVNIKLDITSHNEDYTIVEQYER | |
| | AEGRHSTGGMDELYK | |
| 320 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | dSpCas9-ZNF324 |
| | EDSITVGMVRHQGKIMYVGDVRSVTQKHIQEWGPFDLVIG | |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSENPKKKRKVGIHGVPAADKKYSIGLAIGTNSVG | |
| | WAVITDEYKVPSKKFKVLGNTDRHSIKKNLIGALLFDSGE | |
| | TAEATRLKRTARRRYTRRKNRICYLQEIFSNEMAKVDDSF | |
| | FHRLEESFLVEEDKKHERHPIFGNIVDEVAYHEKYPTIYH | |
| | LRKKLVDSTDKADLRLIYLALAHMIKFRGHFLIEGDLNPD | |
| | NSDVDKLFIQLVQTYNQLFEENPINASGVDAKAILSARLS | |
| | KSRRLENLIAQLPGEKKNGLFGNLIALSLGLTPNFKSNFD | |
| | LAEDAKLQLSKDTYDDDLDNLLAQIGDQYADLFLAAKNLS | |
| | DAILLSDILRVNTEITKAPLSASMIKRYDEHHQDLTLLKA | |
| | LVRQQLPEKYKEIFFDQSKNGYAGYIDGGASQEEFYKFIK | |
| | PILEKMDGTEELLVKLNREDLLRKQRTFDNGSIPHQIHLG | |
| | ELHAILRRQEDFYPFLKDNREKIEKILTFRIPYYVGPLAR | |
| | GNSRFAWMTRKSEETITPWNFEEVVDKGASAQSFIERMTN | |
| | FDKNLPNEKVLPKHSLLYEYFTVYNELTKVKYVTEGMRKP | |
| | AFLSGEQKKAIVDLLFKTNRKVTVKQLKEDYFKKIECFDS | |
| | VEISGVEDRFNASLGTYHDLLKIIKDKDFLDNEENEDILE | |
| | DIVLTLTLFEDREMIEERLKTYAHLFDDKVMKQLKRRRYT | |
| | GWGRLSRKLINGIRDKQSGKTILDFLKSDGFANRNFMQLI | |
| | HDDSLTFKEDIQKAQVSGQGDSLHEHIANLAGSPAIKKGI | |
| | LQTVKVVDELVKVMGRHKPENIVIEMARENQTTQKGQKNS | |
| | RERMKRIEEGIKELGSQILKEHPVENTQLQNEKLYLYYLQ | |
| | NGRDMYVDQELDINRLSDYDVDAIVPQSFLKDDSIDNKVL | |
| | TRSDKNRGKSDNVPSEEVVKKMKNYWRQLLNAKLITQRKF | |
| | DNLTKAERGGLSELDKAGFIKRQLVETRQITKHVAQILDS | |
| | RMNTKYDENDKLIREVKVITLKSKLVSDFRKDFQFYKVRE | |
| | INNYHHAHDAYLNAVVGTALIKKYPKLESEFVYGDYKVYD | |
| | VRKMIAKSEQEIGKATAKYFFYSNIMNFFKTEITLANGEI | |
| | RKRPLIETNGETGEIVWDKGRDFATVRKVLSMPQVNIVKK | |
| | TEVQTGGFSKESILPKRNSDKLIARKKDWDPKKYGGFDSP | |
| | TVAYSVLVVAKVEKGKSKKLKSVKELLGITIMERSSFEKN | |
| | PIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYT | |
| | STKEVLDATLIHQSITGLYETRIDLSQLGGDSGGKRPAAT | |
| | KKAGQAKKKKASAFEDVAVYFSQEEWGLLDTAQRALYRRV | |
| | MLDNFALVASLGLSTSRPRVVIQLERGEEPWVPSGTDTTL | |
| | SRTTYRRRNPGSWSLTEDRDVSGPKKKRKVASATNFSLLK | |
| | QAGDVEENPGPMVSKGEEDNMAIIKEFMRFKVHMEGSVNG | |
| | HEFEIEGEGEGRPYEGTQTAKLKVTKGGPLPFAWDILSPQ | |
| | FMYGSKAYVKHPADIPDYLKLSFPEGFKWERVMNFEDGGV | |
| | VTVTQDSSLQDGEFIYKVKLRGTNFPSDGPVMQKKTMGWE | |
| | ASSERMYPEDGALKGEIKQRLKLKDGGHYDAEVKTTYKAK | |
| | KPVQLPGAYNVNIKLDITSHNEDYTIVEQYERAEGRHSTG | |
| | GMDELYK | |
| 321 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | dSpCas9-EZH2 |
| | EDSITVGMVRHQGKIMYVGDVRSVTQKHIQEWGPFDLVIG | |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSENPKKKRKVGIHGVPAADKKYSIGLAIGTNSVG | |
| | WAVITDEYKVPSKKFKVLGNTDRHSIKKNLIGALLFDSGE | |
| | TAEATRLKRTARRRYTRRKNRICYLQEIFSNEMAKVDDSF | |
| | FHRLEESFLVEEDKKHERHPIFGNIVDEVAYHEKYPTIYH | |
| | LRKKLVDSTDKADLRLIYLALAHMIKFRGHFLIEGDLNPD | |
| | NSDVDKLFIQLVQTYNQLFEENPINASGVDAKAILSARLS | |
| | KSRRLENLIAQLPGEKKNGLFGNLIALSLGLTPNFKSNFD | |
| | LAEDAKLQLSKDTYDDDLDNLLAQIGDQYADLFLAAKNLS | |
| | DAILLSDILRVNTEITKAPLSASMIKRYDEHHQDLTLLKA | |
| | LVRQQLPEKYKEIFFDQSKNGYAGYIDGGASQEEFYKFIK | |
| | PILEKMDGTEELLVKLNREDLLRKQRTFDNGSIPHQIHLG | |
| | ELHAILRRQEDFYPFLKDNREKIEKILTFRIPYYVGPLAR | |
| | GNSRFAWMTRKSEETITPWNFEEVVDKGASAQSFIERMTN | |
| | FDKNLPNEKVLPKHSLLYEYFTVYNELTKVKYVTEGMRKP | |
| | AFLSGEQKKAIVDLLFKTNRKVTVKQLKEDYFKKIECFDS | |
| | VEISGVEDRFNASLGTYHDLLKIIKDKDFLDNEENEDILE | |
| | DIVLTLTLFEDREMIEERLKTYAHLFDDKVMKQLKRRRYT | |
| | GWGRLSRKLINGIRDKQSGKTILDFLKSDGFANRNFMQLI | |
| | HDDSLTFKEDIQKAQVSGQGDSLHEHIANLAGSPAIKKGI | |
| | LQTVKVVDELVKVMGRHKPENIVIEMARENQTTQKGQKNS | |
| | RERMKRIEEGIKELGSQILKEHPVENTQLQNEKLYLYYLQ | |
| | NGRDMYVDQELDINRLSDYDVDAIVPQSFLKDDSIDNKVL | |
| | TRSDKNRGKSDNVPSEEVVKKMKNYWRQLLNAKLITQRKF | |
| | DNLTKAERGGLSELDKAGFIKRQLVETRQITKHVAQILDS | |
| | RMNTKYDENDKLIREVKVITLKSKLVSDFRKDFQFYKVRE | |
| | INNYHHAHDAYLNAVVGTALIKKYPKLESEFVYGDYKVYD | |
| | VRKMIAKSEQEIGKATAKYFFYSNIMNFFKTEITLANGEI | |
| | RKRPLIETNGETGEIVWDKGRDFATVRKVLSMPQVNIVKK | |
| | TEVQTGGFSKESILPKRNSDKLIARKKDWDPKKYGGFDSP | |
| | TVAYSVLVVAKVEKGKSKKLKSVKELLGITIMERSSFEKN | |
| | PIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYT | |
| | STKEVLDATLIHQSITGLYETRIDLSQLGGDSGGKRPAAT | |
| | KKAGQAKKKKASSTGGSGGSGGSGGSGGSGRPGQTGKKSE | |
| | KGPVCWRKRVKSEYMRLRQLKRFRRADEVKTMFSSNRQKI | |
| | LERTETLNQEWKQRRIQPVHIMTSVSSLRGTRECSVTSDL | |
| | DFPAQVIPLKTLNAVASVPIMYSWSPLQQNFMVEDETVLH | |
| | NIPYMGDEVLDQDGTFIEELIKNYDGKVHGDRECGFINDE | |
| | IFVELVNALGQYNDDDDDDDGDDPDEREEKQKDLEDNRDD | |
| | KETCPPRKFPADKIFEAISSMFPDKGTAEELKEKYKELTE | |
| | QQLPGALPPECTPNIDGPNAKSVQREQSLHSFHTLFCRRC | |
| | FKYDCFLHPFHATPNTYKRKNTETALDNKPCGPQCYQHLE | |
| | GAKEFAAALTAERIKTPPKRPGGRRRGRLPNNSSRPSTPT | |
| | ISVLESKDTDSDREAGTETGGENNDKEEEEKKDETSSSSE | |
| | ANSRCQTPIKMKPNIEPPENVEWSGAEASMFRVLIGTYYD | |
| | NFCAIARLIGTKTCRQVYEFRVKESSIIAPVPTEDVDTPP | |
| | RKKKRKHRLWAAHCRKIQLKKDGSSNHVYNYQPCDHPRQP | |
| | CDSSCPCVIAQNFCEKFCQCSSECQNRFPGCRCKAQCNTK | |
| | QCPCYLAVRECDPDLCLTCGAADHWDSKNVSCKNCSIQRG | |
| | SKKHLLLAPSDVAGWGIFIKDPVQKNEFISEYCGEIISQD | |
| | EADRRGKVYDKYMCSFLFNLNNDFVVDATRKGNKIRFANH | |
| | SVNPNCYAKVMMVNGDHRIGIFAKRAIQTGEELFFDYRYS | |
| | QADALKYVGIEREMEIPPKKKRKVASATNFSLLKQAGDVE | |
| | ENPGPMVSKGEEDNMAIIKEFMRFKVHMEGSVNGHEFEIE | |
| | GEGEGRPYEGTQTAKLKVTKGGPLPFAWDILSPQFMYGSK | |
| | AYVKHPADIPDYLKLSFPEGFKWERVMNFEDGGVVTVTQD | |
| | SSLQDGEFIYKVKLRGTNFPSDGPVMQKKTMGWEASSERM | |
| | YPEDGALKGEIKQRLKLKDGGHYDAEVKTTYKAKKPVQLP | |
| | GAYNVNIKLDITSHNEDYTIVEQYERAEGRHSTGGMDELY | |
| | K | |
| 322 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | KOX1(2-99)- |
| | EDSITVGMVRHQGKIMYVGDVRSVTQKHIQEWGPFDLVIG | dSpCas9 |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSEDAKSLTAWSRTLVTFKDVFVDFTREEWKLLDT | |
| | AQQILYRNVMLENYKNLVSLGYQLTKPDVILRLEKGEEPW | |
| | LVEREIHQETHPDSETAFEIKSSVNPKKKRKVGIHGVPAA | |
| | DKKYSIGLAIGTNSVGWAVITDEYKVPSKKFKVLGNTDRH | |
| | SIKKNLIGALLFDSGETAEATRLKRTARRRYTRRKNRICY | |
| | LQEIFSNEMAKVDDSFFHRLEESFLVEEDKKHERHPIFGN | |
| | IVDEVAYHEKYPTIYHLRKKLVDSTDKADLRLIYLALAHM | |
| | IKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLAQ | |
| | IGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIE | |
| | KILTFRIPYYVGPLARGNSRFAWMTRKSEETITPWNFEEV | |
| | VDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKII | |
| | KDKDFLDNEENEDILEDIVLTLTLFEDREMIEERLKTYAH | |
| | LFDDKVMKQLKRRRYTGWGRLSRKLINGIRDKQSGKTILD | |
| | FLKSDGFANRNFMQLIHDDSLTFKEDIQKAQVSGQGDSLH | |
| | EHIANLAGSPAIKKGILQTVKVVDELVKVMGRHKPENIVI | |
| | EMARENQTTQKGQKNSRERMKRIEEGIKELGSQILKEHPV | |
| | ENTQLQNEKLYLYYLQNGRDMYVDQELDINRLSDYDVDAI | |
| | VPQSFLKDDSIDNKVLTRSDKNRGKSDNVPSEEVVKKMKN | |
| | YWRQLLNAKLITQRKFDNLTKAERGGLSELDKAGFIKRQL | |
| | VETRQITKHVAQILDSRMNTKYDENDKLIREVKVITLKSK | |
| | LVSDFRKDFQFYKVREINNYHHAHDAYLNAVVGTALIKKY | |
| | PKLESEFVYGDYKVYDVRKMIAKSEQEIGKATAKYFFYSN | |
| | IMNFFKTEITLANGEIRKRPLIETNGETGEIVWDKGRDFA | |
| | TVRKVLSMPQVNIVKKTEVQTGGFSKESILPKRNSDKLIA | |
| | RKKDWDPKKYGGFDSPTVAYSVLVVAKVEKGKSKKLKSVK | |
| | ELLGITIMERSSFEKNPIDFLEAKGYKEVKKDLIIKLPKY | |
| | SLFELENGRKRMLASAGELQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKRYTSTKEVLDATLIHQSITGLYETRID | |
| | LSQLGGDSGGKRPAATKKAGQAKKKKASATNFSLLKQAGD | |
| | VEENPGPMVSKGEEDNMAIIKEFMRFKVHMEGSVNGHEFE | |
| | IEGEGEGRPYEGTQTAKLKVTKGGPLPFAWDILSPQFMYG | |
| | SKAYVKHPADIPDYLKLSFPEGFKWERVMNFEDGGVVTVT | |
| | QDSSLQDGEFIYKVKLRGTNFPSDGPVMQKKTMGWEASSE | |
| | RMYPEDGALKGEIKQRLKLKDGGHYDAEVKTTYKAKKPVQ | |
| | LPGAYNVNIKLDITSHNEDYTIVEQYERAEGRHSTGGMDE | |
| | LYK | |
| 323 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | KOX1(1-72)- |
| | EDSITVGMVRHQGKIMYVGDVRSVTQKHIQEWGPFDLVIG | dSpCas9 |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSEMDAKSLTAWSRTLVTFKDVFVDFTREEWKLLD | |
| | TAQQIVYRNVMLENYKNLVSLGYQLTKPDVILRLEKGEEP | |
| | NPKKKRKVGIHGVPAADKKYSIGLAIGTNSVGWAVITDEY | |
| | KVPSKKFKVLGNTDRHSIKKNLIGALLFDSGETAEATRLK | |
| | RTARRRYTRRKNRICYLQEIFSNEMAKVDDSFFHRLEESF | |
| | LVEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLENL | |
| | IAQLPGEKKNGLFGNLIALSLGLTPNFKSNFDLAEDAKLQ | |
| | LSKDTYDDDLDNLLAQIGDQYADLFLAAKNLSDAILLSDI | |
| | LRVNTEITKAPLSASMIKRYDEHHQDLTLLKALVRQQLPE | |
| | KYKEIFFDQSKNGYAGYIDGGASQEEFYKFIKPILEKMDG | |
| | TEELLVKLNREDLLRKQRTFDNGSIPHQIHLGELHAILRR | |
| | QEDFYPFLKDNREKIEKILTFRIPYYVGPLARGNSRFAWM | |
| | TRKSEETITPWNFEEVVDKGASAQSFIERMTNFDKNLPNE | |
| | KVLPKHSLLYEYFTVYNELTKVKYVTEGMRKPAFLSGEQK | |
| | KAIVDLLFKTNRKVTVKQLKEDYFKKIECFDSVEISGVED | |
| | RFNASLGTYHDLLKIIKDKDFLDNEENEDILEDIVLTLTL | |
| | FEDREMIEERLKTYAHLFDDKVMKQLKRRRYTGWGRLSRK | |
| | LINGIRDKQSGKTILDFLKSDGFANRNFMQLIHDDSLTFK | |
| | EDIQKAQVSGQGDSLHEHIANLAGSPAIKKGILQTVKVVD | |
| | ELVKVMGRHKPENIVIEMARENQTTQKGQKNSRERMKRIE | |
| | EGIKELGSQILKEHPVENTQLQNEKLYLYYLQNGRDMYVD | |
| | QELDINRLSDYDVDAIVPQSFLKDDSIDNKVLTRSDKNRG | |
| | KSDNVPSEEVVKKMKNYWRQLLNAKLITQRKFDNLTKAER | |
| | GGLSELDKAGFIKRQLVETRQITKHVAQILDSRMNTKYDE | |
| | NDKLIREVKVITLKSKLVSDFRKDFQFYKVREINNYHHAH | |
| | DAYLNAVVGTALIKKYPKLESEFVYGDYKVYDVRKMIAKS | |
| | EQEIGKATAKYFFYSNIMNFFKTEITLANGEIRKRPLIET | |
| | NGETGEIVWDKGRDFATVRKVLSMPQVNIVKKTEVQTGGF | |
| | SKESILPKRNSDKLIARKKDWDPKKYGGFDSPTVAYSVLV | |
| | VAKVEKGKSKKLKSVKELLGITIMERSSFEKNPIDFLEAK | |
| | GYKEVKKDLIIKLPKYSLFELENGRKRMLASAGELQKGNE | |
| | LALPSKYVNFLYLASHYEKLKGSPEDNEQKQLFVEQHKHY | |
| | LDEIIEQISEFSKRVILADANLDKVLSAYNKHRDKPIREQ | |
| | AENIIHLFTLTNLGAPAAFKYFDTTIDRKRYTSTKEVLDA | |
| | TLIHQSITGLYETRIDLSQLGGDSGGKRPAATKKAGQAKK | |
| | KKASATNFSLLKQAGDVEENPGPMVSKGEEDNMAIIKEFM | |
| | RFKVHMEGSVNGHEFEIEGEGEGRPYEGTQTAKLKVTKGG | |
| | PLPFAWDILSPQFMYGSKAYVKHPADIPDYLKLSFPEGFK | |
| | WERVMNFEDGGVVTVTQDSSLQDGEFIYKVKLRGTNFPSD | |
| | GPVMQKKTMGWEASSERMYPEDGALKGEIKQRLKLKDGGH | |
| | YDAEVKTTYKAKKPVQLPGAYNVNIKLDITSHNEDYTIVE | |
| | QYERAEGRHSTGGMDELYK | |
| 324 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | ZIM3-dSpCas9 |
| | EDSITVGMVRHQGKIMYVGDVRSVTQKHIQEWGPFDLVIG | |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSENNSQGRVTFEDVTVNFTQGEWQRLNPEQRNLY | |
| | RDVMLENYSNLVSVGQGETTKPDVILRLEQGKEPWLEEEE | |
| | VLGSGRAEKNGDIGGQIWKPKDVKESLNPKKKRKVGIHGV | |
| | PAADKKYSIGLAIGTNSVGWAVITDEYKVPSKKFKVLGNT | |
| | DRHSIKKNLIGALLFDSGETAEATRLKRTARRRYTRRKNR | |
| | ICYLQEIFSNEMAKVDDSFFHRLEESFLVEEDKKHERHPI | |
| | FGNIVDEVAYHEKYPTIYHLRKKLVDSTDKADLRLIYLAL | |
| | AHMIKFRGHFLIEGDLNPDNSDVDKLFIQLVQTYNQLFEE | |
| | NPINASGVDAKAILSARLSKSRRLENLIAQLPGEKKNGLF | |
| | GNLIALSLGLTPNFKSNFDLAEDAKLQLSKDTYDDDLDNL | |
| | LAQIGDQYADLFLAAKNLSDAILLSDILRVNTEITKAPLS | |
| | ASMIKRYDEHHQDLTLLKALVRQQLPEKYKEIFFDQSKNG | |
| | YAGYIDGGASQEEFYKFIKPILEKMDGTEELLVKLNREDL | |
| | LRKQRTFDNGSIPHQIHLGELHAILRRQEDFYPFLKDNRE | |
| | KIEKILTFRIPYYVGPLARGNSRFAWMTRKSEETITPWNF | |
| | EEVVDKGASAQSFIERMTNFDKNLPNEKVLPKHSLLYEYF | |
| | TVYNELTKVKYVTEGMRKPAFLSGEQKKAIVDLLFKTNRK | |
| | VTVKQLKEDYFKKIECFDSVEISGVEDRFNASLGTYHDLL | |
| | KIIKDKDFLDNEENEDILEDIVLTLTLFEDREMIEERLKT | |
| | YAHLFDDKVMKQLKRRRYTGWGRLSRKLINGIRDKQSGKT | |
| | ILDFLKSDGFANRNFMQLIHDDSLTFKEDIQKAQVSGQGD | |
| | SLHEHIANLAGSPAIKKGILQTVKVVDELVKVMGRHKPEN | |
| | IVIEMARENQTTQKGQKNSRERMKRIEEGIKELGSQILKE | |
| | HPVENTQLQNEKLYLYYLQNGRDMYVDQELDINRLSDYDV | |
| | DAIVPQSFLKDDSIDNKVLTRSDKNRGKSDNVPSEEVVKK | |
| | MKNYWRQLLNAKLITQRKFDNLTKAERGGLSELDKAGFIK | |
| | RQLVETRQITKHVAQILDSRMNTKYDENDKLIREVKVITL | |
| | KSKLVSDFRKDFQFYKVREINNYHHAHDAYLNAVVGTALI | |
| | KKYPKLESEFVYGDYKVYDVRKMIAKSEQEIGKATAKYFF | |
| | YSNIMNFFKTEITLANGEIRKRPLIETNGETGEIVWDKGR | |
| | DFATVRKVLSMPQVNIVKKTEVQTGGFSKESILPKRNSDK | |
| | LIARKKDWDPKKYGGFDSPTVAYSVLVVAKVEKGKSKKLK | |
| | SVKELLGITIMERSSFEKNPIDFLEAKGYKEVKKDLIIKL | |
| | PKYSLFELENGRKRMLASAGELQKGNELALPSKYVNFLYL | |
| | ASHYEKLKGSPEDNEQKQLFVEQHKHYLDEIIEQISEFSK | |
| | RVILADANLDKVLSAYNKHRDKPIREQAENIIHLFTLTNL | |
| | GAPAAFKYFDTTIDRKRYTSTKEVLDATLIHQSITGLYET | |
| | RIDLSQLGGDSGGKRPAATKKAGQAKKKKASATNFSLLKQ | |
| | AGDVEENPGPMVSKGEEDNMAIIKEFMRFKVHMEGSVNGH | |
| | EFEIEGEGEGRPYEGTQTAKLKVTKGGPLPFAWDILSPQF | |
| | MYGSKAYVKHPADIPDYLKLSFPEGFKWERVMNFEDGGVV | |
| | TVTQDSSLQDGEFIYKVKLRGTNFPSDGPVMQKKTMGWEA | |
| | SSERMYPEDGALKGEIKQRLKLKDGGHYDAEVKTTYKAKK | |
| | PVQLPGAYNVNIKLDITSHNEDYTIVEQYERAEGRHSTGG | |
| | MDELYK | |
| 325 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | ZNF324-dSpCas9 |
| | EDSITVGMVRHQGKIMYVGDVRSVTQKHIQEWGPFDLVIG | |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSEAFEDVAVYFSQEEWGLLDTAQRALYRRVMLDN | |
| | FALVASLGLSTSRPRVVIQLERGEEPWVPSGTDTTLSRTT | |
| | YRRRNPGSWSLTEDRDVSGNPKKKRKVGIHGVPAADKKYS | |
| | IGLAIGTNSVGWAVITDEYKVPSKKFKVLGNTDRHSIKKN | |
| | LIGALLFDSGETAEATRLKRTARRRYTRRKNRICYLQEIF | |
| | SNEMAKVDDSFFHRLEESFLVEEDKKHERHPIFGNIVDEV | |
| | AYHEKYPTIYHLRKKLVDSTDKADLRLIYLALAHMIKFRG | |
| | HFLIEGDLNPDNSDVDKLFIQLVQTYNQLFEENPINASGV | |
| | DAKAILSARLSKSRRLENLIAQLPGEKKNGLFGNLIALSL | |
| | GLTPNFKSNFDLAEDAKLQLSKDTYDDDLDNLLAQIGDQY | |
| | ADLFLAAKNLSDAILLSDILRVNTEITKAPLSASMIKRYD | |
| | EHHQDLTLLKALVRQQLPEKYKEIFFDQSKNGYAGYIDGG | |
| | ASQEEFYKFIKPILEKMDGTEELLVKLNREDLLRKQRTFD | |
| | NGSIPHQIHLGELHAILRRQEDFYPFLKDNREKIEKILTF | |
| | RIPYYVGPLARGNSRFAWMTRKSEETITPWNFEEVVDKGA | |
| | SAQSFIERMTNFDKNLPNEKVLPKHSLLYEYFTVYNELTK | |
| | VKYVTEGMRKPAFLSGEQKKAIVDLLFKTNRKVTVKQLKE | |
| | DYFKKIECFDSVEISGVEDRFNASLGTYHDLLKIIKDKDF | |
| | LDNEENEDILEDIVLTLTLFEDREMIEERLKTYAHLFDDK | |
| | VMKQLKRRRYTGWGRLSRKLINGIRDKQSGKTILDFLKSD | |
| | GFANRNFMQLIHDDSLTFKEDIQKAQVSGQGDSLHEHIAN | |
| | LAGSPAIKKGILQTVKVVDELVKVMGRHKPENIVIEMARE | |
| | NQTTQKGQKNSRERMKRIEEGIKELGSQILKEHPVENTQL | |
| | QNEKLYLYYLQNGRDMYVDQELDINRLSDYDVDAIVPQSF | |
| | LKDDSIDNKVLTRSDKNRGKSDNVPSEEVVKKMKNYWRQL | |
| | LNAKLITQRKFDNLTKAERGGLSELDKAGFIKRQLVETRQ | |
| | ITKHVAQILDSRMNTKYDENDKLIREVKVITLKSKLVSDF | |
| | RKDFQFYKVREINNYHHAHDAYLNAVVGTALIKKYPKLES | |
| | EFVYGDYKVYDVRKMIAKSEQEIGKATAKYFFYSNIMNFF | |
| | KTEITLANGEIRKRPLIETNGETGEIVWDKGRDFATVRKV | |
| | LSMPQVNIVKKTEVQTGGFSKESILPKRNSDKLIARKKDW | |
| | DPKKYGGFDSPTVAYSVLVVAKVEKGKSKKLKSVKELLGI | |
| | TIMERSSFEKNPIDFLEAKGYKEVKKDLIIKLPKYSLFEL | |
| | ENGRKRMLASAGELQKGNELALPSKYVNFLYLASHYEKLK | |
| | GSPEDNEQKQLFVEQHKHYLDEIIEQISEFSKRVILADAN | |
| | LDKVLSAYNKHRDKPIREQAENIIHLFTLTNLGAPAAFKY | |
| | FDTTIDRKRYTSTKEVLDATLIHQSITGLYETRIDLSQLG | |
| | GDSGGKRPAATKKAGQAKKKKASATNFSLLKQAGDVEENP | |
| | GPMVSKGEEDNMAIIKEFMRFKVHMEGSVNGHEFEIEGEG | |
| | EGRPYEGTQTAKLKVTKGGPLPFAWDILSPQFMYGSKAYV | |
| | KHPADIPDYLKLSFPEGFKWERVMNFEDGGVVTVTQDSSL | |
| | QDGEFIYKVKLRGTNFPSDGPVMQKKTMGWEASSERMYPE | |
| | DGALKGEIKQRLKLKDGGHYDAEVKTTYKAKKPVQLPGAY | |
| | NVNIKLDITSHNEDYTIVEQYERAEGRHSTGGMDELYK | |
| 326 | MDYKDHDGDYKDHDIDYKDDDDKHVNHDQEFDPPKVYPPV | D3AL-XTEN80- |
| | PAEKRKPIRVLSLFDGIATGLLVLKDLGIQVDRYIASEVC | EZH2-dSpCas9 |
| | EDSITVGMVRHQGKIMYVGDVRSVTQKHIQEWGPFDLVIG | |
| | GSPCNDLSIVNPARKGLYEGTGRLFFEFYRLLHDARPKEG | |
| | DDRPFFWLFENVVAMGVSDKRDISRFLESNPVMIDAKEVS | |
| | AAHRARYFWGNLPGMNRPLASTVNDKLELQECLEHGRIAK | |
| | FSKVRTITTRSNSIKQGKDQHFPVFMNEKEDILWCTEMER | |
| | VFGFPVHYTDVSNMSRLARQRLLGRSWSVPVIRHLFAPLK | |
| | EYFACVSSGNSNANSRGPSFSSGLVPLSLRGSHMGPMEIY | |
| | KTVSAWKRQPVRVLSLFRNIDKVLKSLGFLESGSGSGGGT | |
| | LKYVEDVTNVVRRDVEKWGPFDLVYGSTQPLGSSCDRCPG | |
| | WYMFQFHRILQYALPRQESQRPFFWIFMDNLLLTEDDQET | |
| | TTRFLQTEAVTLQDVRGRDYQNAMRVWSNIPGLKSKHAPL | |
| | TPKEEEYLQAQVRSRSKLDAPKVDLLVKNCLLPLREYFKY | |
| | FSQNSLPLGGPSSGAPPPSGGSPAGSPTSTEEGTSESATP | |
| | ESGPGTSTEPSEGSAPGSPAGSPTSTEEGTSTEPSEGSAP | |
| | GTSTEPSEGQTGKKSEKGPVCWRKRVKSEYMRLRQLKRFR | |
| | RADEVKTMFSSNRQKILERTETLNQEWKQRRIQPVHIMTS | |
| | VSSLRGTRECSVTSDLDFPAQVIPLKTLNAVASVPIMYSW | |
| | SPLQQNFMVEDETVLHNIPYMGDEVLDQDGTFIEELIKNY | |
| | DGKVHGDRECGFINDEIFVELVNALGQYNDDDDDDDGDDP | |
| | DEREEKQKDLEDNRDDKETCPPRKFPADKIFEAISSMFPD | |
| | KGTAEELKEKYKELTEQQLPGALPPECTPNIDGPNAKSVQ | |
| | REQSLHSFHTLFCRRCFKYDCFLHPFHATPNTYKRKNTET | |
| | ALDNKPCGPQCYQHLEGAKEFAAALTAERIKTPPKRPGGR | |
| | RRGRLPNNSSRPSTPTISVLESKDTDSDREAGTETGGENN | |
| | DKEEEEKKDETSSSSEANSRCQTPIKMKPNIEPPENVEWS | |
| | GAEASMFRVLIGTYYDNFCAIARLIGTKTCRQVYEFRVKE | |
| | SSIIAPVPTEDVDTPPRKKKRKHRLWAAHCRKIQLKKDGS | |
| | SNHVYNYQPCDHPRQPCDSSCPCVIAQNFCEKFCQCSSEC | |
| | QNRFPGCRCKAQCNTKQCPCYLAVRECDPDLCLTCGAADH | |
| | WDSKNVSCKNCSIQRGSKKHLLLAPSDVAGWGIFIKDPVQ | |
| | KNEFISEYCGEIISQDEADRRGKVYDKYMCSFLFNLNNDF | |
| | VVDATRKGNKIRFANHSVNPNCYAKVMMVNGDHRIGIFAK | |
| | RAIQTGEELFFDYRYSQADALKYVGIEREMEIPSTGGSGG | |
| | SGGSGGSGGSGRPNPKKKRKVGIHGVPAADKKYSIGLAIG | |
| | TNSVGWAVITDEYKVPSKKFKVLGNTDRHSIKKNLIGALL | |
| | FDSGETAEATRLKRTARRRYTRRKNRICYLQEIFSNEMAK | |
| | VDDSFFHRLEESFLVEEDKKHERHPIFGNIVDEVAYHEKY | |
| | PTIYHLRKKLVDSTDKADLRLIYLALAHMIKFRGHFLIEG | |
| | DLNPDNSDVDKLFIQLVQTYNQLFEENPINASGVDAKAIL | |
| | SARLSKSRRLENLIAQLPGEKKNGLFGNLIALSLGLTPNF | |
| | KSNFDLAEDAKLQLSKDTYDDDLDNLLAQIGDQYADLFLA | |
| | AKNLSDAILLSDILRVNTEITKAPLSASMIKRYDEHHQDL | |
| | TLLKALVRQQLPEKYKEIFFDQSKNGYAGYIDGGASQEEF | |
| | YKFIKPILEKMDGTEELLVKLNREDLLRKQRTFDNGSIPH | |
| | QIHLGELHAILRRQEDFYPFLKDNREKIEKILTFRIPYYV | |
| | GPLARGNSRFAWMTRKSEETITPWNFEEVVDKGASAQSFI | |
| | ERMTNFDKNLPNEKVLPKHSLLYEYFTVYNELTKVKYVTE | |
| | GMRKPAFLSGEQKKAIVDLLFKTNRKVTVKQLKEDYFKKI | |
| | ECFDSVEISGVEDRFNASLGTYHDLLKIIKDKDFLDNEEN | |
| | EDILEDIVLTLTLFEDREMIEERLKTYAHLFDDKVMKQLK | |
| | RRRYTGWGRLSRKLINGIRDKQSGKTILDFLKSDGFANRN | |
| | FMQLIHDDSLTFKEDIQKAQVSGQGDSLHEHIANLAGSPA | |
| | IKKGILQTVKVVDELVKVMGRHKPENIVIEMARENQTTQK | |
| | GQKNSRERMKRIEEGIKELGSQILKEHPVENTQLQNEKLY | |
| | LYYLQNGRDMYVDQELDINRLSDYDVDAIVPQSFLKDDSI | |
| | DNKVLTRSDKNRGKSDNVPSEEVVKKMKNYWRQLLNAKLI | |
| | TQRKFDNLTKAERGGLSELDKAGFIKRQLVETRQITKHVA | |
| | QILDSRMNTKYDENDKLIREVKVITLKSKLVSDFRKDFQF | |
| | YKVREINNYHHAHDAYLNAVVGTALIKKYPKLESEFVYGD | |
| | YKVYDVRKMIAKSEQEIGKATAKYFFYSNIMNFFKTEITL | |
| | ANGEIRKRPLIETNGETGEIVWDKGRDFATVRKVLSMPQV | |
| | NIVKKTEVQTGGFSKESILPKRNSDKLIARKKDWDPKKYG | |
| | GFDSPTVAYSVLVVAKVEKGKSKKLKSVKELLGITIMERS | |
| | SFEKNPIDFLEAKGYKEVKKDLIIKLPKYSLFELENGRKR | |
| | MLASAGELQKGNELALPSKYVNFLYLASHYEKLKGSPEDN | |
| | EQKQLFVEQHKHYLDEIIEQISEFSKRVILADANLDKVLS | |
| | AYNKHRDKPIREQAENIIHLFTLTNLGAPAAFKYFDTTID | |
| | RKRYTSTKEVLDATLIHQSITGLYETRIDLSQLGGDSGGK | |
| | RPAATKKAGQAKKKKASATNFSLLKQAGDVEENPGPMVSK | |
| | GEEDNMAIIKEFMRFKVHMEGSVNGHEFEIEGEGEGRPYE | |
| | GTQTAKLKVTKGGPLPFAWDILSPQFMYGSKAYVKHPADI | |
| | PDYLKLSFPEGFKWERVMNFEDGGVVTVTQDSSLQDGEFI | |
| | YKVKLRGTNFPSDGPVMQKKTMGWEASSERMYPEDGALKG | |
| | EIKQRLKLKDGGHYDAEVKTTYKAKKPVQLPGAYNVNIKL | |
| | DITSHNEDYTIVEQYERAEGRHSTGGMDELYK | |
| 327 | MDYKDHDGDYKDHDIDYKDDDDKHVVLRRRKDWNMRLQDF | D3BL-XTEN80- |
| | FTTDPDLEEFQEPPKLYPAIPAAKRRPIRVLSLFDGIATG | dSpCas9-KOX1(2- |
| | YLVLKELGIKVEKYIASEVCAESIAVGTVKHEGQIKYVND | 99) |
| | VRKITKKNIEEWGPFDLVIGGSPCNDLSNVNPARKGLYEG | |
| | TGRLFFEFYHLLNYTRPKEGDNRPFFWMFENVVAMKVNDK | |
| | KDISRFLACNPVMIDAIKVSAAHRARYFWGNLPGMNRPVM | |
| | ASKNDKLELQDCLEFSRTAKLKKVQTITTKSNSIRQGKNQ | |
| | LFPVVMNGKDDVLWCTELERIFGFPAHYTDVSNMGRGARQ | |
| | KLLGRSWSVPVIRHLFAPLKDYFACESSGNSNANSRGPSF | |
| | SSGLVPLSLRGSHMGPMEIYKTVSAWKRQPVRVLSLFRNI | |
| | DKVLKSLGFLESGSGSGGGTLKYVEDVTNVVRRDVEKWGP | |
| | FDLVYGSTQPLGSSCDRCPGWYMFQFHRILQYALPRQESQ | |
| | RPFFWIFMDNLLLTEDDQETTTRFLQTEAVTLQDVRGRDY | |
| | QNAMRVWSNIPGLKSKHAPLTPKEEEYLQAQVRSRSKLDA | |
| | PKVDLLVKNCLLPLREYFKYFSQNSLPLGGPSSGAPPPSG | |
| | GSPAGSPTSTEEGTSESATPESGPGTSTEPSEGSAPGSPA | |
| | GSPTSTEEGTSTEPSEGSAPGTSTEPSENPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGDSGGKRPAATKKAGQAKKKKASDAKSLTAW | |
| | SRTLVTFKDVFVDFTREEWKLLDTAQQILYRNVMLENYKN | |
| | LVSLGYQLTKPDVILRLEKGEEPWLVEREIHQETHPDSET | |
| | AFEIKSSVPKKKRKVASATNFSLLKQAGDVEENPGPMVSK | |
| | GEEDNMAIIKEFMRFKVHMEGSVNGHEFEIEGEGEGRPYE | |
| | GTQTAKLKVTKGGPLPFAWDILSPQFMYGSKAYVKHPADI | |
| | PDYLKLSFPEGFKWERVMNFEDGGVVTVTQDSSLQDGEFI | |
| | YKVKLRGTNFPSDGPVMQKKTMGWEASSERMYPEDGALKG | |
| | EIKQRLKLKDGGHYDAEVKTTYKAKKPVQLPGAYNVNIKL | |
| | DITSHNEDYTIVEQYERAEGRHSTGGMDELYK | |
| 328 | MDYKDHDGDYKDHDIDYKDDDDKHVVLRRRKDWNMRLQDF | D3BL-XTEN80- |
| | FTTDPDLEEFQEPPKLYPAIPAAKRRPIRVLSLFDGIATG | dSpCas9-KOX1(1- |
| | YLVLKELGIKVEKYIASEVCAESIAVGTVKHEGQIKYVND | 72) |
| | VRKITKKNIEEWGPFDLVIGGSPCNDLSNVNPARKGLYEG | |
| | TGRLFFEFYHLLNYTRPKEGDNRPFFWMFENVVAMKVNDK | |
| | KDISRFLACNPVMIDAIKVSAAHRARYFWGNLPGMNRPVM | |
| | ASKNDKLELQDCLEFSRTAKLKKVQTITTKSNSIRQGKNQ | |
| | LFPVVMNGKDDVLWCTELERIFGFPAHYTDVSNMGRGARQ | |
| | KLLGRSWSVPVIRHLFAPLKDYFACESSGNSNANSRGPSF | |
| | SSGLVPLSLRGSHMGPMEIYKTVSAWKRQPVRVLSLFRNI | |
| | DKVLKSLGFLESGSGSGGGTLKYVEDVTNVVRRDVEKWGP | |
| | FDLVYGSTQPLGSSCDRCPGWYMFQFHRILQYALPRQESQ | |
| | RPFFWIFMDNLLLTEDDQETTTRFLQTEAVTLQDVRGRDY | |
| | QNAMRVWSNIPGLKSKHAPLTPKEEEYLQAQVRSRSKLDA | |
| | PKVDLLVKNCLLPLREYFKYFSQNSLPLGGPSSGAPPPSG | |
| | GSPAGSPTSTEEGTSESATPESGPGTSTEPSEGSAPGSPA | |
| | GSPTSTEEGTSTEPSEGSAPGTSTEPSENPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGDSGGKRPAATKKAGQAKKKKASMDAKSLTA | |
| | WSRTLVTFKDVFVDFTREEWKLLDTAQQIVYRNVMLENYK | |
| | NLVSLGYQLTKPDVILRLEKGEEPPKKKRKVASATNFSLL | |
| | KQAGDVEENPGPMVSKGEEDNMAIIKEFMRFKVHMEGSVN | |
| | GHEFEIEGEGEGRPYEGTQTAKLKVTKGGPLPFAWDILSP | |
| | QFMYGSKAYVKHPADIPDYLKLSFPEGFKWERVMNFEDGG | |
| | VVTVTQDSSLQDGEFIYKVKLRGTNFPSDGPVMQKKTMGW | |
| | EASSERMYPEDGALKGEIKQRLKLKDGGHYDAEVKTTYKA | |
| | KKPVQLPGAYNVNIKLDITSHNEDYTIVEQYERAEGRHST | |
| | GGMDELYK | |
| 329 | MDYKDHDGDYKDHDIDYKDDDDKHVVLRRRKDWNMRLQDF | D3BL-XTEN80- |
| | FTTDPDLEEFQEPPKLYPAIPAAKRRPIRVLSLFDGIATG | dSpCas9-ZIM3 |
| | YLVLKELGIKVEKYIASEVCAESIAVGTVKHEGQIKYVND | |
| | VRKITKKNIEEWGPFDLVIGGSPCNDLSNVNPARKGLYEG | |
| | TGRLFFEFYHLLNYTRPKEGDNRPFFWMFENVVAMKVNDK | |
| | KDISRFLACNPVMIDAIKVSAAHRARYFWGNLPGMNRPVM | |
| | ASKNDKLELQDCLEFSRTAKLKKVQTITTKSNSIRQGKNQ | |
| | LFPVVMNGKDDVLWCTELERIFGFPAHYTDVSNMGRGARQ | |
| | KLLGRSWSVPVIRHLFAPLKDYFACESSGNSNANSRGPSF | |
| | SSGLVPLSLRGSHMGPMEIYKTVSAWKRQPVRVLSLFRNI | |
| | DKVLKSLGFLESGSGSGGGTLKYVEDVTNVVRRDVEKWGP | |
| | FDLVYGSTQPLGSSCDRCPGWYMFQFHRILQYALPRQESQ | |
| | RPFFWIFMDNLLLTEDDQETTTRFLQTEAVTLQDVRGRDY | |
| | QNAMRVWSNIPGLKSKHAPLTPKEEEYLQAQVRSRSKLDA | |
| | PKVDLLVKNCLLPLREYFKYFSQNSLPLGGPSSGAPPPSG | |
| | GSPAGSPTSTEEGTSESATPESGPGTSTEPSEGSAPGSPA | |
| | GSPTSTEEGTSTEPSEGSAPGTSTEPSENPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGDSGGKRPAATKKAGQAKKKKASNNSQGRVT | |
| | FEDVTVNFTQGEWQRLNPEQRNLYRDVMLENYSNLVSVGQ | |
| | GETTKPDVILRLEQGKEPWLEEEEVLGSGRAEKNGDIGGQ | |
| | IWKPKDVKESLPKKKRKVASATNFSLLKQAGDVEENPGPM | |
| | VSKGEEDNMAIIKEFMRFKVHMEGSVNGHEFEIEGEGEGR | |
| | PYEGTQTAKLKVTKGGPLPFAWDILSPQFMYGSKAYVKHP | |
| | ADIPDYLKLSFPEGFKWERVMNFEDGGVVTVTQDSSLQDG | |
| | EFIYKVKLRGTNFPSDGPVMQKKTMGWEASSERMYPEDGA | |
| | LKGEIKQRLKLKDGGHYDAEVKTTYKAKKPVQLPGAYNVN | |
| | IKLDITSHNEDYTIVEQYERAEGRHSTGGMDELYK | |
| 330 | MDYKDHDGDYKDHDIDYKDDDDKHVVLRRRKDWNMRLQDF | D3BL-XTEN80- |
| | FTTDPDLEEFQEPPKLYPAIPAAKRRPIRVLSLFDGIATG | dSpCas9-ZNF324 |
| | YLVLKELGIKVEKYIASEVCAESIAVGTVKHEGQIKYVND | |
| | VRKITKKNIEEWGPFDLVIGGSPCNDLSNVNPARKGLYEG | |
| | TGRLFFEFYHLLNYTRPKEGDNRPFFWMFENVVAMKVNDK | |
| | KDISRFLACNPVMIDAIKVSAAHRARYFWGNLPGMNRPVM | |
| | ASKNDKLELQDCLEFSRTAKLKKVQTITTKSNSIRQGKNQ | |
| | LFPVVMNGKDDVLWCTELERIFGFPAHYTDVSNMGRGARQ | |
| | KLLGRSWSVPVIRHLFAPLKDYFACESSGNSNANSRGPSF | |
| | SSGLVPLSLRGSHMGPMEIYKTVSAWKRQPVRVLSLFRNI | |
| | DKVLKSLGFLESGSGSGGGTLKYVEDVTNVVRRDVEKWGP | |
| | FDLVYGSTQPLGSSCDRCPGWYMFQFHRILQYALPRQESQ | |
| | RPFFWIFMDNLLLTEDDQETTTRFLQTEAVTLQDVRGRDY | |
| | QNAMRVWSNIPGLKSKHAPLTPKEEEYLQAQVRSRSKLDA | |
| | PKVDLLVKNCLLPLREYFKYFSQNSLPLGGPSSGAPPPSG | |
| | GSPAGSPTSTEEGTSESATPESGPGTSTEPSEGSAPGSPA | |
| | GSPTSTEEGTSTEPSEGSAPGTSTEPSENPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGDSGGKRPAATKKAGQAKKKKASAFEDVAVY | |
| | FSQEEWGLLDTAQRALYRRVMLDNFALVASLGLSTSRPRV | |
| | VIQLERGEEPWVPSGTDTTLSRTTYRRRNPGSWSLTEDRD | |
| | VSGPKKKRKVASATNFSLLKQAGDVEENPGPMVSKGEEDN | |
| | MAIIKEFMRFKVHMEGSVNGHEFEIEGEGEGRPYEGTQTA | |
| | KLKVTKGGPLPFAWDILSPQFMYGSKAYVKHPADIPDYLK | |
| | LSFPEGFKWERVMNFEDGGVVTVTQDSSLQDGEFIYKVKL | |
| | RGTNFPSDGPVMQKKTMGWEASSERMYPEDGALKGEIKQR | |
| | LKLKDGGHYDAEVKTTYKAKKPVQLPGAYNVNIKLDITSH | |
| | NEDYTIVEQYERAEGRHSTGGMDELYK | |
| 331 | MDYKDHDGDYKDHDIDYKDDDDKHVVLRRRKDWNMRLQDF | D3BL-XTEN80- |
| | FTTDPDLEEFQEPPKLYPAIPAAKRRPIRVLSLFDGIATG | dSpCas9-EZH2 |
| | YLVLKELGIKVEKYIASEVCAESIAVGTVKHEGQIKYVND | |
| | VRKITKKNIEEWGPFDLVIGGSPCNDLSNVNPARKGLYEG | |
| | TGRLFFEFYHLLNYTRPKEGDNRPFFWMFENVVAMKVNDK | |
| | KDISRFLACNPVMIDAIKVSAAHRARYFWGNLPGMNRPVM | |
| | ASKNDKLELQDCLEFSRTAKLKKVQTITTKSNSIRQGKNQ | |
| | LFPVVMNGKDDVLWCTELERIFGFPAHYTDVSNMGRGARQ | |
| | KLLGRSWSVPVIRHLFAPLKDYFACESSGNSNANSRGPSF | |
| | SSGLVPLSLRGSHMGPMEIYKTVSAWKRQPVRVLSLFRNI | |
| | DKVLKSLGFLESGSGSGGGTLKYVEDVTNVVRRDVEKWGP | |
| | FDLVYGSTQPLGSSCDRCPGWYMFQFHRILQYALPRQESQ | |
| | RPFFWIFMDNLLLTEDDQETTTRFLQTEAVTLQDVRGRDY | |
| | QNAMRVWSNIPGLKSKHAPLTPKEEEYLQAQVRSRSKLDA | |
| | PKVDLLVKNCLLPLREYFKYFSQNSLPLGGPSSGAPPPSG | |
| | GSPAGSPTSTEEGTSESATPESGPGTSTEPSEGSAPGSPA | |
| | GSPTSTEEGTSTEPSEGSAPGTSTEPSENPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGDSGGKRPAATKKAGQAKKKKASSTGGSGGS | |
| | GGSGGSGGSGRPGQTGKKSEKGPVCWRKRVKSEYMRLRQL | |
| | KRFRRADEVKTMFSSNRQKILERTETLNQEWKQRRIQPVH | |
| | IMTSVSSLRGTRECSVTSDLDFPAQVIPLKTLNAVASVPI | |
| | MYSWSPLQQNFMVEDETVLHNIPYMGDEVLDQDGTFIEEL | |
| | IKNYDGKVHGDRECGFINDEIFVELVNALGQYNDDDDDDD | |
| | GDDPDEREEKQKDLEDNRDDKETCPPRKFPADKIFEAISS | |
| | MFPDKGTAEELKEKYKELTEQQLPGALPPECTPNIDGPNA | |
| | KSVQREQSLHSFHTLFCRRCFKYDCFLHPFHATPNTYKRK | |
| | NTETALDNKPCGPQCYQHLEGAKEFAAALTAERIKTPPKR | |
| | PGGRRRGRLPNNSSRPSTPTISVLESKDTDSDREAGTETG | |
| | GENNDKEEEEKKDETSSSSEANSRCQTPIKMKPNIEPPEN | |
| | VEWSGAEASMFRVLIGTYYDNFCAIARLIGTKTCRQVYEF | |
| | RVKESSIIAPVPTEDVDTPPRKKKRKHRLWAAHCRKIQLK | |
| | KDGSSNHVYNYQPCDHPRQPCDSSCPCVIAQNFCEKFCQC | |
| | SSECQNRFPGCRCKAQCNTKQCPCYLAVRECDPDLCLTCG | |
| | AADHWDSKNVSCKNCSIQRGSKKHLLLAPSDVAGWGIFIK | |
| | DPVQKNEFISEYCGEIISQDEADRRGKVYDKYMCSFLFNL | |
| | NNDFVVDATRKGNKIRFANHSVNPNCYAKVMMVNGDHRIG | |
| | IFAKRAIQTGEELFFDYRYSQADALKYVGIEREMEIPPKK | |
| | KRKVASATNFSLLKQAGDVEENPGPMVSKGEEDNMAIIKE | |
| | FMRFKVHMEGSVNGHEFEIEGEGEGRPYEGTQTAKLKVTK | |
| | GGPLPFAWDILSPQFMYGSKAYVKHPADIPDYLKLSFPEG | |
| | FKWERVMNFEDGGVVTVTQDSSLQDGEFIYKVKLRGTNFP | |
| | SDGPVMQKKTMGWEASSERMYPEDGALKGEIKQRLKLKDG | |
| | GHYDAEVKTTYKAKKPVQLPGAYNVNIKLDITSHNEDYTI | |
| | VEQYERAEGRHSTGGMDELYK | |
| 332 | PKKKRKVGIHGVPAADKKYSIGLAIGTNSVGWAVITDEYK | dSpCas9-KOX1(2- |
| | VPSKKFKVLGNTDRHSIKKNLIGALLFDSGETAEATRLKR | 99) - No FLAG, |
| | TARRRYTRRKNRICYLQEIFSNEMAKVDDSFFHRLEESFL | P2A-mCherry |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDSGGKRPAATKKAGQAKKK | |
| | KASDAKSLTAWSRTLVTFKDVFVDFTREEWKLLDTAQQIL | |
| | YRNVMLENYKNLVSLGYQLTKPDVILRLEKGEEPWLVERE | |
| | IHQETHPDSETAFEIKSSVPKKKRKV | |
| 333 | PKKKRKVGIHGVPAADKKYSIGLAIGTNSVGWAVITDEYK | dSpCas9-KOX1(1- |
| | VPSKKFKVLGNTDRHSIKKNLIGALLFDSGETAEATRLKR | 72) - No FLAG, |
| | TARRRYTRRKNRICYLQEIFSNEMAKVDDSFFHRLEESFL | P2A-mCherry |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQ\LFEENPINASGVDAKAILSARLSKSRRLENL | |
| | IAQLPGEKKNGLFGNLIALSLGLTPNFKSNFDLAEDAKLQ | |
| | LSKDTYDDDLDNLLAQIGDQYADLFLAAKNLSDAILLSDI | |
| | LRVNTEITKAPLSASMIKRYDEHHQDLTLLKALVRQQLPE | |
| | KYKEIFFDQSKNGYAGYIDGGASQEEFYKFIKPILEKMDG | |
| | TEELLVKLNREDLLRKQRTFDNGSIPHQIHLGELHAILRR | |
| | QEDFYPFLKDNREKIEKILTFRIPYYVGPLARGNSRFAWM | |
| | TRKSEETITPWNFEEVVDKGASAQSFIERMTNFDKNLPNE | |
| | KVLPKHSLLYEYFTVYNELTKVKYVTEGMRKPAFLSGEQK | |
| | KAIVDLLFKTNRKVTVKQLKEDYFKKIECFDSVEISGVED | |
| | RFNASLGTYHDLLKIIKDKDFLDNEENEDILEDIVLTLTL | |
| | FEDREMIEERLKTYAHLFDDKVMKQLKRRRYTGWGRLSRK | |
| | LINGIRDKQSGKTILDFLKSDGFANRNFMQLIHDDSLTFK | |
| | EDIQKAQVSGQGDSLHEHIANLAGSPAIKKGILQTVKVVD | |
| | ELVKVMGRHKPENIVIEMARENQTTQKGQKNSRERMKRIE | |
| | EGIKELGSQILKEHPVENTQLQNEKLYLYYLQNGRDMYVD | |
| | QELDINRLSDYDVDAIVPQSFLKDDSIDNKVLTRSDKNRG | |
| | KSDNVPSEEVVKKMKNYWRQLLNAKLITQRKFDNLTKAER | |
| | GGLSELDKAGFIKRQLVETRQITKHVAQILDSRMNTKYDE | |
| | NDKLIREVKVITLKSKLVSDFRKDFQFYKVREINNYHHAH | |
| | DAYLNAVVGTALIKKYPKLESEFVYGDYKVYDVRKMIAKS | |
| | EQEIGKATAKYFFYSNIMNFFKTEITLANGEIRKRPLIET | |
| | NGETGEIVWDKGRDFATVRKVLSMPQVNIVKKTEVQTGGF | |
| | SKESILPKRNSDKLIARKKDWDPKKYGGFDSPTVAYSVLV | |
| | VAKVEKGKSKKLKSVKELLGITIMERSSFEKNPIDFLEAK | |
| | GYKEVKKDLIIKLPKYSLFELENGRKRMLASAGELQKGNE | |
| | LALPSKYVNFLYLASHYEKLKGSPEDNEQKQLFVEQHKHY | |
| | LDEIIEQISEFSKRVILADANLDKVLSAYNKHRDKPIREQ | |
| | AENIIHLFTLTNLGAPAAFKYFDTTIDRKRYTSTKEVLDA | |
| | TLIHQSITGLYETRIDLSQLGGDSGGKRPAATKKAGQAKK | |
| | KKASMDAKSLTAWSRTLVTFKDVFVDFTREEWKLLDTAQQ | |
| | IVYRNVMLENYKNLVSLGYQLTKPDVILRLEKGEEPPKKK | |
| | RKV | |
| 334 | PKKKRKVGIHGVPAADKKYSIGLAIGTNSVGWAVITDEYK | dSpCas9-ZIM3 - |
| | VPSKKFKVLGNTDRHSIKKNLIGALLFDSGETAEATRLKR | No FLAG, P2A- |
| | TARRRYTRRKNRICYLQEIFSNEMAKVDDSFFHRLEESFL | mCherry |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDSGGKRPAATKKAGQAKKK | |
| | KASNNSQGRVTFEDVTVNFTQGEWQRLNPEQRNLYRDVML | |
| | ENYSNLVSVGQGETTKPDVILRLEQGKEPWLEEEEVLGSG | |
| | RAEKNGDIGGQIWKPKDVKESLPKKKRKV | |
| 335 | PKKKRKVGIHGVPAADKKYSIGLAIGTNSVGWAVITDEYK | dSpCas9-ZNF324 - |
| | VPSKKFKVLGNTDRHSIKKNLIGALLFDSGETAEATRLKR | No FLAG, P2A- |
| | TARRRYTRRKNRICYLQEIFSNEMAKVDDSFFHRLEESFL | mCherry |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDSGGKRPAATKKAGQAKKK | |
| | KASAFEDVAVYFSQEEWGLLDTAQRALYRRVMLDNFALVA | |
| | SLGLSTSRPRVVIQLERGEEPWVPSGTDTTLSRTTYRRRN | |
| | PGSWSLTEDRDVSGPKKKRKV | |
| 336 | PKKKRKVGIHGVPAADKKYSIGLAIGTNSVGWAVITDEYK | dSpCas9-EZH2 - |
| | VPSKKFKVLGNTDRHSIKKNLIGALLFDSGETAEATRLKR | No FLAG, P2A- |
| | TARRRYTRRKNRICYLQEIFSNEMAKVDDSFFHRLEESFL | mCherry |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDSGGKRPAATKKAGQAKKK | |
| | KASSTGGSGGSGGSGGSGGSGRPGQTGKKSEKGPVCWRKR | |
| | VKSEYMRLRQLKRFRRADEVKTMFSSNRQKILERTETLNQ | |
| | EWKQRRIQPVHIMTSVSSLRGTRECSVTSDLDFPAQVIPL | |
| | KTLNAVASVPIMYSWSPLQQNFMVEDETVLHNIPYMGDEV | |
| | LDQDGTFIEELIKNYDGKVHGDRECGFINDEIFVELVNAL | |
| | GQYNDDDDDDDGDDPDEREEKQKDLEDNRDDKETCPPRKF | |
| | PADKIFEAISSMFPDKGTAEELKEKYKELTEQQLPGALPP | |
| | ECTPNIDGPNAKSVQREQSLHSFHTLFCRRCFKYDCFLHP | |
| | FHATPNTYKRKNTETALDNKPCGPQCYQHLEGAKEFAAAL | |
| | TAERIKTPPKRPGGRRRGRLPNNSSRPSTPTISVLESKDT | |
| | DSDREAGTETGGENNDKEEEEKKDETSSSSEANSRCQTPI | |
| | KMKPNIEPPENVEWSGAEASMFRVLIGTYYDNFCAIARLI | |
| | GTKTCRQVYEFRVKESSIIAPVPTEDVDTPPRKKKRKHRL | |
| | WAAHCRKIQLKKDGSSNHVYNYQPCDHPRQPCDSSCPCVI | |
| | AQNFCEKFCQCSSECQNRFPGCRCKAQCNTKQCPCYLAVR | |
| | ECDPDLCLTCGAADHWDSKNVSCKNCSIQRGSKKHLLLAP | |
| | SDVAGWGIFIKDPVQKNEFISEYCGEIISQDEADRRGKVY | |
| | DKYMCSFLFNLNNDFVVDATRKGNKIRFANHSVNPNCYAK | |
| | VMMVNGDHRIGIFAKRAIQTGEELFFDYRYSQADALKYVG | |
| | IEREMEIPPKKKRKV | |
| 337 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-KOX1(2- |
| | QKHIQEWGPFDLVIGGSPCNDLSIVNPARKGLYEGTGRLF | 99) - No FLAG, |
| | FEFYRLLHDARPKEGDDRPFFWLFENVVAMGVSDKRDISR | P2A-mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASDAKSLTAWSRTLV | |
| | TFKDVFVDFTREEWKLLDTAQQILYRNVMLENYKNLVSLG | |
| | YQLTKPDVILRLEKGEEPWLVEREIHQETHPDSETAFEIK | |
| | SSVPKKKRKV | |
| 338 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-KOX1(1- |
| | QKHIQEWGPFDLVIGGSPCNDLSIVNPARKGLYEGTGRLF | 72) - No FLAG, |
| | FEFYRLLHDARPKEGDDRPFFWLFENVVAMGVSDKRDISR | P2A-mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASMDAKSLTAWSRTL | |
| | VTFKDVFVDFTREEWKLLDTAQQIVYRNVMLENYKNLVSL | |
| | GYQLTKPDVILRLEKGEEPPKKKRKV | |
| 339 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-ZIM3 - |
| | QKHIQEWGPFDLVIGGSPCNDLSIVNPARKGLYEGTGRLF | No FLAG, P2A- |
| | FEFYRLLHDARPKEGDDRPFFWLFENVVAMGVSDKRDISR | mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTE | |
| 340 | AVTLQDVRGRDYQNAMRVWSNIPGLKSKHAPLTPKEEEYL | D3AL-XTEN80- |
| | QAQVRSRSKLDAPKVDLLVKNCLLPLREYFKYFSQNSLPL | dSpCas9-ZNF324 - |
| | GGPSSGAPPPSGGSPAGSPTSTEEGTSESATPESGPGTST | No FLAG, P2A- |
| | EPSEGSAPGSPAGSPTSTEEGTSTEPSEGSAPGTSTEPSE | mCherry |
| | NPKKKRKVGIHGVPAADKKYSIGLAIGTNSVGWAVITDEY | |
| | KVPSKKFKVLGNTDRHSIKKNLIGALLFDSGETAEATRLK | |
| | RTARRRYTRRKNRICYLQEIFSNEMAKVDDSFFHRLEESF | |
| | LVEEDKKHERHPIFGNIVDEVAYHEKYPTIYHLRKKLVDS | |
| | TDKADLRLIYLALAHMIKFRGHFLIEGDLNPDNSDVDKLF | |
| | IQLVQTYNQLFEENPINASGVDAKAILSARLSKSRRLENL | |
| | IAQLPGEKKNGLFGNLIALSLGLTPNFKSNFDLAEDAKLQ | |
| | LSKDTYDDDLDNLLAQIGDQYADLFLAAKNLSDAILLSDI | |
| | LRVNTEITKAPLSASMIKRYDEHHQDLTLLKALVRQQLPE | |
| | KYKEIFFDQSKNGYAGYIDGGASQEEFYKFIKPILEKMDG | |
| | TEELLVKLNREDLLRKQRTFDNGSIPHQIHLGELHAILRR | |
| | QEDFYPFLKDNREKIEKILTFRIPYYVGPLARGNSRFAWM | |
| | TRKSEETITPWNFEEVVDKGASAQSFIERMTNFDKNLPNE | |
| | KVLPKHSLLYEYFTVYNELTKVKYVTEGMRKPAFLSGEQK | |
| | KAIVDLLFKTNRKVTVKQLKEDYFKKIECFDSVEISGVED | |
| | RFNASLGTYHDLLKIIKDKDFLDNEENEDILEDIVLTLTL | |
| | FEDREMIEERLKTYAHLFDDKVMKQLKRRRYTGWGRLSRK | |
| | LINGIRDKQSGKTILDFLKSDGFANRNFMQLIHDDSLTFK | |
| | EDIQKAQVSGQGDSLHEHIANLAGSPAIKKGILQTVKVVD | |
| | ELVKVMGRHKPENIVIEMARENQTTQKGQKNSRERMKRIE | |
| | EGIKELGSQILKEHPVENTQLQNEKLYLYYLQNGRDMYVD | |
| | QELDINRLSDYDVDAIVPQSFLKDDSIDNKVLTRSDKNRG | |
| | KSDNVPSEEVVKKMKNYWRQLLNAKLITQRKFDNLTKAER | |
| | GGLSELDKAGFIKRQLVETRQITKHVAQILDSRMNTKYDE | |
| | NDKLIREVKVITLKSKLVSDFRKDFQFYKVREINNYHHAH | |
| | DAYLNAVVGTALIKKYPKLESEFVYGDYKVYDVRKMIAKS | |
| | EQEIGKATAKYFFYSNIMNFFKTEITLANGEIRKRPLIET | |
| | NGETGEIVWDKGRDFATVRKVLSMPQVNIVKKTEVQTGGF | |
| | SKESILPKRNSDKLIARKKDWDPKKYGGFDSPTVAYSVLV | |
| | VAKVEKGKSKKLKSVKELLGITIMERSSFEKNPIDFLEAK | |
| | GYKEVKKDLIIKLPKYSLFELENGRKRMLASAGELQKGNE | |
| | LALPSKYVNFLYLASHYEKLKGSPEDNEQKQLFVEQHKHY | |
| | LDEIIEQISEFSKRVILADANLDKVLSAYNKHRDKPIREQ | |
| | AENIIHLFTLTNLGAPAAFKYFDTTIDRKRYTSTKEVLDA | |
| | TLIHQSITGLYETRIDLSQLGGDSGGKRPAATKKAGQAKK | |
| | KKASNNSQGRVTFEDVTVNFTQGEWQRLNPEQRNLYRDVM | |
| | LENYSNLVSVGQGETTKPDVILRLEQGKEPWLEEEEVLGS | |
| | GRAEKNGDIGGQIWKPKDVKESLPKKKRKVNHDQEFDPPK | |
| | VYPPVPAEKRKPIRVLSLFDGIATGLLVLKDLGIQVDRYI | |
| | ASEVCEDSITVGMVRHQGKIMYVGDVRSVTQKHIQEWGPF | |
| | DLVIGGSPCNDLSIVNPARKGLYEGTGRLFFEFYRLLHDA | |
| | RPKEGDDRPFFWLFENVVAMGVSDKRDISRFLESNPVMID | |
| | AKEVSAAHRARYFWGNLPGMNRPLASTVNDKLELQECLEH | |
| | GRIAKFSKVRTITTRSNSIKQGKDQHFPVFMNEKEDILWC | |
| | TEMERVFGFPVHYTDVSNMSRLARQRLLGRSWSVPVIRHL | |
| | FAPLKEYFACVSSGNSNANSRGPSFSSGLVPLSLRGSHMG | |
| | PMEIYKTVSAWKRQPVRVLSLFRNIDKVLKSLGFLESGSG | |
| | SGGGTLKYVEDVTNVVRRDVEKWGPFDLVYGSTQPLGSSC | |
| | DRCPGWYMFQFHRILQYALPRQESQRPFFWIFMDNLLLTE | |
| | DDQETTTRFLQTEAVTLQDVRGRDYQNAMRVWSNIPGLKS | |
| | KHAPLTPKEEEYLQAQVRSRSKLDAPKVDLLVKNCLLPLR | |
| | EYFKYFSQNSLPLGGPSSGAPPPSGGSPAGSPTSTEEGTS | |
| | ESATPESGPGTSTEPSEGSAPGSPAGSPTSTEEGTSTEPS | |
| | EGSAPGTSTEPSENPKKKRKVGIHGVPAADKKYSIGLAIG | |
| | TNSVGWAVITDEYKVPSKKFKVLGNTDRHSIKKNLIGALL | |
| | FDSGETAEATRLKRTARRRYTRRKNRICYLQEIFSNEMAK | |
| | VDDSFFHRLEESFLVEEDKKHERHPIFGNIVDEVAYHEKY | |
| | PTIYHLRKKLVDSTDKADLRLIYLALAHMIKFRGHFLIEG | |
| | DLNPDNSDVDKLFIQLVQTYNQLFEENPINASGVDAKAIL | |
| | SARLSKSRRLENLIAQLPGEKKNGLFGNLIALSLGLTPNF | |
| | KSNFDLAEDAKLQLSKDTYDDDLDNLLAQIGDQYADLFLA | |
| | AKNLSDAILLSDILRVNTEITKAPLSASMIKRYDEHHQDL | |
| | TLLKALVRQQLPEKYKEIFFDQSKNGYAGYIDGGASQEEF | |
| | YKFIKPILEKMDGTEELLVKLNREDLLRKQRTFDNGSIPH | |
| | QIHLGELHAILRRQEDFYPFLKDNREKIEKILTFRIPYYV | |
| | GPLARGNSRFAWMTRKSEETITPWNFEEVVDKGASAQSFI | |
| | ERMTNFDKNLPNEKVLPKHSLLYEYFTVYNELTKVKYVTE | |
| | GMRKPAFLSGEQKKAIVDLLFKTNRKVTVKQLKEDYFKKI | |
| | ECFDSVEISGVEDRFNASLGTYHDLLKIIKDKDFLDNEEN | |
| | EDILEDIVLTLTLFEDREMIEERLKTYAHLFDDKVMKQLK | |
| | RRRYTGWGRLSRKLINGIRDKQSGKTILDFLKSDGFANRN | |
| | FMQLIHDDSLTFKEDIQKAQVSGQGDSLHEHIANLAGSPA | |
| | IKKGILQTVKVVDELVKVMGRHKPENIVIEMARENQTTQK | |
| | GQKNSRERMKRIEEGIKELGSQILKEHPVENTQLQNEKLY | |
| | LYYLQNGRDMYVDQELDINRLSDYDVDAIVPQSFLKDDSI | |
| | DNKVLTRSDKNRGKSDNVPSEEVVKKMKNYWRQLLNAKLI | |
| | TQRKFDNLTKAERGGLSELDKAGFIKRQLVETRQITKHVA | |
| | QILDSRMNTKYDENDKLIREVKVITLKSKLVSDFRKDFQF | |
| | YKVREINNYHHAHDAYLNAVVGTALIKKYPKLESEFVYGD | |
| | YKVYDVRKMIAKSEQEIGKATAKYFFYSNIMNFFKTEITL | |
| | ANGEIRKRPLIETNGETGEIVWDKGRDFATVRKVLSMPQV | |
| | NIVKKTEVQTGGFSKESILPKRNSDKLIARKKDWDPKKYG | |
| | GFDSPTVAYSVLVVAKVEKGKSKKLKSVKELLGITIMERS | |
| | SFEKNPIDFLEAKGYKEVKKDLIIKLPKYSLFELENGRKR | |
| | MLASAGELQKGNELALPSKYVNFLYLASHYEKLKGSPEDN | |
| | EQKQLFVEQHKHYLDEIIEQISEFSKRVILADANLDKVLS | |
| | AYNKHRDKPIREQAENIIHLFTLTNLGAPAAFKYFDTTID | |
| | RKRYTSTKEVLDATLIHQSITGLYETRIDLSQLGGDSGGK | |
| | RPAATKKAGQAKKKKASAFEDVAVYFSQEEWGLLDTAQRA | |
| | LYRRVMLDNFALVASLGLSTSRPRVVIQLERGEEPWVPSG | |
| | TDTTLSRTTYRRRNPGSWSLTEDRDVSGPKKKRKV | |
| 341 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-EZH2 - |
| | QKHIQEWGPFDLVIGGSPCNDLSIVNPARKGLYEGTGRLF | No FLAG, P2A- |
| | FEFYRLLHDARPKEGDDRPFFWLFENVVAMGVSDKRDISR | mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASSTGGSGGSGGSGG | |
| | SGGSGRPGQTGKKSEKGPVCWRKRVKSEYMRLRQLKRFRR | |
| | ADEVKTMFSSNRQKILERTETLNQEWKQRRIQPVHIMTSV | |
| | SSLRGTRECSVTSDLDFPAQVIPLKTLNAVASVPIMYSWS | |
| | PLQQNFMVEDETVLHNIPYMGDEVLDQDGTFIEELIKNYD | |
| | GKVHGDRECGFINDEIFVELVNALGQYNDDDDDDDGDDPD | |
| | EREEKQKDLEDNRDDKETCPPRKFPADKIFEAISSMFPDK | |
| | GTAEELKEKYKELTEQQLPGALPPECTPNIDGPNAKSVQR | |
| | EQSLHSFHTLFCRRCFKYDCFLHPFHATPNTYKRKNTETA | |
| | LDNKPCGPQCYQHLEGAKEFAAALTAERIKTPPKRPGGRR | |
| | RGRLPNNSSRPSTPTISVLESKDTDSDREAGTETGGENND | |
| | KEEEEKKDETSSSSEANSRCQTPIKMKPNIEPPENVEWSG | |
| | AEASMFRVLIGTYYDNFCAIARLIGTKTCRQVYEFRVKES | |
| | SIIAPVPTEDVDTPPRKKKRKHRLWAAHCRKIQLKKDGSS | |
| | NHVYNYQPCDHPRQPCDSSCPCVIAQNFCEKFCQCSSECQ | |
| | NRFPGCRCKAQCNTKQCPCYLAVRECDPDLCLTCGAADHW | |
| | DSKNVSCKNCSIQRGSKKHLLLAPSDVAGWGIFIKDPVQK | |
| | NEFISEYCGEIISQDEADRRGKVYDKYMCSFLFNLNNDFV | |
| | VDATRKGNKIRFANHSVNPNCYAKVMMVNGDHRIGIFAKR | |
| | AIQTGEELFFDYRYSQADALKYVGIEREMEIPPKKKRKV | |
| 342 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | KOX1(2-99)- |
| | QKHIQEWGPFDLVIGGSPCNDLSIVNPARKGLYEGTGRLF | dSpCas9 - No |
| | FEFYRLLHDARPKEGDDRPFFWLFENVVAMGVSDKRDISR | FLAG, P2A- |
| | FLESNPVMIDAKEVSAAHRARYFWGNLPGMNRPLASTVND | mCherry |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEDAKSLTAWSRTLVTFKD | |
| | VFVDFTREEWKLLDTAQQILYRNVMLENYKNLVSLGYQLT | |
| | KPDVILRLEKGEEPWLVEREIHQETHPDSETAFEIKSSVN | |
| | PKKKRKVGIHGVPAADKKYSIGLAIGTNSVGWAVITDEYK | |
| | VPSKKFKVLGNTDRHSIKKNLIGALLFDSGETAEATRLKR | |
| | TARRRYTRRKNRICYLQEIFSNEMAKVDDSFFHRLEESFL | |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGDSGGKRPAATKKAGQAKKK | |
| 343 | K | |
| | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | KOX1(1-72)- |
| | QKHIQEWGPFDLVIGGSPCNDLSIVNPARKGLYEGTGRLF | dSpCas9 - No |
| | FEFYRLLHDARPKEGDDRPFFWLFENVVAMGVSDKRDISR | FLAG, P2A- |
| | FLESNPVMIDAKEVSAAHRARYFWGNLPGMNRPLASTVND | mCherry |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEMDAKSLTAWSRTLVTFK | |
| | DVFVDFTREEWKLLDTAQQIVYRNVMLENYKNLVSLGYQL | |
| | TKPDVILRLEKGEEPNPKKKRKVGIHGVPAADKKYSIGLA | |
| | IGTNSVGWAVITDEYKVPSKKFKVLGNTDRHSIKKNLIGA | |
| | LLFDSGETAEATRLKRTARRRYTRRKNRICYLQEIFSNEM | |
| | AKVDDSFFHRLEESFLVEEDKKHERHPIFGNIVDEVAYHE | |
| | KYPTIYHLRKKLVDSTDKADLRLIYLALAHMIKFRGHFLI | |
| | EGDLNPDNSDVDKLFIQLVQTYNQLFEENPINASGVDAKA | |
| | ILSARLSKSRRLENLIAQLPGEKKNGLFGNLIALSLGLTP | |
| | NFKSNFDLAEDAKLQLSKDTYDDDLDNLLAQIGDQYADLF | |
| | LAAKNLSDAILLSDILRVNTEITKAPLSASMIKRYDEHHQ | |
| | DLTLLKALVRQQLPEKYKEIFFDQSKNGYAGYIDGGASQE | |
| | EFYKFIKPILEKMDGTEELLVKLNREDLLRKQRTFDNGSI | |
| | PHQIHLGELHAILRRQEDFYPFLKDNREKIEKILTFRIPY | |
| | YVGPLARGNSRFAWMTRKSEETITPWNFEEVVDKGASAQS | |
| | FIERMTNFDKNLPNEKVLPKHSLLYEYFTVYNELTKVKYV | |
| | TEGMRKPAFLSGEQKKAIVDLLFKTNRKVTVKQLKEDYFK | |
| | KIECFDSVEISGVEDRFNASLGTYHDLLKIIKDKDFLDNE | |
| | ENEDILEDIVLTLTLFEDREMIEERLKTYAHLFDDKVMKQ | |
| | LKRRRYTGWGRLSRKLINGIRDKQSGKTILDFLKSDGFAN | |
| | RNFMQLIHDDSLTFKEDIQKAQVSGQGDSLHEHIANLAGS | |
| | PAIKKGILQTVKVVDELVKVMGRHKPENIVIEMARENQTT | |
| | QKGQKNSRERMKRIEEGIKELGSQILKEHPVENTQLQNEK | |
| | LYLYYLQNGRDMYVDQELDINRLSDYDVDAIVPQSFLKDD | |
| | SIDNKVLTRSDKNRGKSDNVPSEEVVKKMKNYWRQLLNAK | |
| | LITQRKFDNLTKAERGGLSELDKAGFIKRQLVETRQITKH | |
| | VAQILDSRMNTKYDENDKLIREVKVITLKSKLVSDFRKDF | |
| | QFYKVREINNYHHAHDAYLNAVVGTALIKKYPKLESEFVY | |
| | GDYKVYDVRKMIAKSEQEIGKATAKYFFYSNIMNFFKTEI | |
| | TLANGEIRKRPLIETNGETGEIVWDKGRDFATVRKVLSMP | |
| | QVNIVKKTEVQTGGFSKESILPKRNSDKLIARKKDWDPKK | |
| | YGGFDSPTVAYSVLVVAKVEKGKSKKLKSVKELLGITIME | |
| | RSSFEKNPIDFLEAKGYKEVKKDLIIKLPKYSLFELENGR | |
| | KRMLASAGELQKGNELALPSKYVNFLYLASHYEKLKGSPE | |
| | DNEQKQLFVEQHKHYLDEIIEQISEFSKRVILADANLDKV | |
| | LSAYNKHRDKPIREQAENIIHLFTLTNLGAPAAFKYFDTT | |
| | IDRKRYTSTKEVLDATLIHQSITGLYETRIDLSQLGGDSG | |
| | GKRPAATKKAGQAKKKK | |
| 344 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | ZIM3-dSpCas9 - |
| | QKHIQEWGPFDLVIGGSPCNDLSIVNPARKGLYEGTGRLF | No FLAG, P2A- |
| | FEFYRLLHDARPKEGDDRPFFWLFENVVAMGVSDKRDISR | mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENNSQGRVTFEDVTVNFT | |
| | QGEWQRLNPEQRNLYRDVMLENYSNLVSVGQGETTKPDVI | |
| | LRLEQGKEPWLEEEEVLGSGRAEKNGDIGGQIWKPKDVKE | |
| | SLNPKKKRKVGIHGVPAADKKYSIGLAIGTNSVGWAVITD | |
| | EYKVPSKKFKVLGNTDRHSIKKNLIGALLFDSGETAEATR | |
| | LKRTARRRYTRRKNRICYLQEIFSNEMAKVDDSFFHRLEE | |
| | SFLVEEDKKHERHPIFGNIVDEVAYHEKYPTIYHLRKKLV | |
| | DSTDKADLRLIYLALAHMIKFRGHFLIEGDLNPDNSDVDK | |
| | LFIQLVQTYNQLFEENPINASGVDAKAILSARLSKSRRLE | |
| | NLIAQLPGEKKNGLFGNLIALSLGLTPNFKSNFDLAEDAK | |
| | LQLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEKM | |
| | DGTEELLVKLNREDLLRKQRTFDNGSIPHQIHLGELHAIL | |
| | RRQEDFYPFLKDNREKIEKILTFRIPYYVGPLARGNSRFA | |
| | WMTRKSEETITPWNFEEVVDKGASAQSFIERMTNFDKNLP | |
| | NEKVLPKHSLLYEYFTVYNELTKVKYVTEGMRKPAFLSGE | |
| | QKKAIVDLLFKTNRKVTVKQLKEDYFKKIECFDSVEISGV | |
| | EDRFNASLGTYHDLLKIIKDKDFLDNEENEDILEDIVLTL | |
| | TLFEDREMIEERLKTYAHLFDDKVMKQLKRRRYTGWGRLS | |
| | RKLINGIRDKQSGKTILDFLKSDGFANRNFMQLIHDDSLT | |
| | FKEDIQKAQVSGQGDSLHEHIANLAGSPAIKKGILQTVKV | |
| | VDELVKVMGRHKPENIVIEMARENQTTQKGQKNSRERMKR | |
| | IEEGIKELGSQILKEHPVENTQLQNEKLYLYYLQNGRDMY | |
| | VDQELDINRLSDYDVDAIVPQSFLKDDSIDNKVLTRSDKN | |
| | RGKSDNVPSEEVVKKMKNYWRQLLNAKLITQRKFDNLTKA | |
| | ERGGLSELDKAGFIKRQLVETRQITKHVAQILDSRMNTKY | |
| | DENDKLIREVKVITLKSKLVSDFRKDFQFYKVREINNYHH | |
| | AHDAYLNAVVGTALIKKYPKLESEFVYGDYKVYDVRKMIA | |
| | KSEQEIGKATAKYFFYSNIMNFFKTEITLANGEIRKRPLI | |
| | ETNGETGEIVWDKGRDFATVRKVLSMPQVNIVKKTEVQTG | |
| | GFSKESILPKRNSDKLIARKKDWDPKKYGGFDSPTVAYSV | |
| | LVVAKVEKGKSKKLKSVKELLGITIMERSSFEKNPIDFLE | |
| | AKGYKEVKKDLIIKLPKYSLFELENGRKRMLASAGELQKG | |
| | NELALPSKYVNFLYLASHYEKLKGSPEDNEQKQLFVEQHK | |
| | HYLDEIIEQISEFSKRVILADANLDKVLSAYNKHRDKPIR | |
| | EQAENIIHLFTLTNLGAPAAFKYFDTTIDRKRYTSTKEVL | |
| | DATLIHQSITGLYETRIDLSQLGGDSGGKRPAATKKAGQA | |
| | KKKK | |
| 345 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | ZNF324-dSpCas9 - |
| | QKHIQEWGPFDLVIGGSPCNDLSIVNPARKGLYEGTGRLF | No FLAG, P2A- |
| | FEFYRLLHDARPKEGDDRPFFWLFENVVAMGVSDKRDISR | mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEAFEDVAVYFSQEEWGLL | |
| | DTAQRALYRRVMLDNFALVASLGLSTSRPRVVIQLERGEE | |
| | PWVPSGTDTTLSRTTYRRRNPGSWSLTEDRDVSGNPKKKR | |
| | KVGIHGVPAADKKYSIGLAIGTNSVGWAVITDEYKVPSKK | |
| | FKVLGNTDRHSIKKNLIGALLFDSGETAEATRLKRTARRR | |
| | YTRRKNRICYLQEIFSNEMAKVDDSFFHRLEESFLVEEDK | |
| | KHERHPIFGNIVDEVAYHEKYPTIYHLRKKLVDSTDKADL | |
| | RLIYLALAHMIKFRGHFLIEGDLNPDNSDVDKLFIQLVQT | |
| | YNQLFEENPINASGVDAKAILSARLSKSRRLENLIAQLPG | |
| | EKKNGLFGNLIALSLGLTPNFKSNFDLAEDAKLQLSKDTY | |
| | DDDLDNLLAQIGDQYADLFLAAKNLSDAILLSDILRVNTE | |
| | ITKAPLSASMIKRYDEHHQDLTLLKALVRQQLPEKYKEIF | |
| | FDQSKNGYAGYIDGGASQEEFYKFIKPILEKMDGTEELLV | |
| | KLNREDLLRKQRTFDNGSIPHQIHLGELHAILRRQEDFYP | |
| | FLKDNREKIEKILTFRIPYYVGPLARGNSRFAWMTRKSEE | |
| | TITPWNFEEVVDKGASAQSFIERMTNFDKNLPNEKVLPKH | |
| | SLLYEYFTVYNELTKVKYVTEGMRKPAFLSGEQKKAIVDL | |
| | LFKTNRKVTVKQLKEDYFKKIECFDSVEISGVEDRFNASL | |
| | GTYHDLLKIIKDKDFLDNEENEDILEDIVLTLTLFEDREM | |
| | IEERLKTYAHLFDDKVMKQLKRRRYTGWGRLSRKLINGIR | |
| | DKQSGKTILDFLKSDGFANRNFMQLIHDDSLTFKEDIQKA | |
| | QVSGQGDSLHEHIANLAGSPAIKKGILQTVKVVDELVKVM | |
| | GRHKPENIVIEMARENQTTQKGQKNSRERMKRIEEGIKEL | |
| | GSQILKEHPVENTQLQNEKLYLYYLQNGRDMYVDQELDIN | |
| | RLSDYDVDAIVPQSFLKDDSIDNKVLTRSDKNRGKSDNVP | |
| | SEEVVKKMKNYWRQLLNAKLITQRKFDNLTKAERGGLSEL | |
| | DKAGFIKRQLVETRQITKHVAQILDSRMNTKYDENDKLIR | |
| | EVKVITLKSKLVSDFRKDFQFYKVREINNYHHAHDAYLNA | |
| | VVGTALIKKYPKLESEFVYGDYKVYDVRKMIAKSEQEIGK | |
| | ATAKYFFYSNIMNFFKTEITLANGEIRKRPLIETNGETGE | |
| | IVWDKGRDFATVRKVLSMPQVNIVKKTEVQTGGFSKESIL | |
| | PKRNSDKLIARKKDWDPKKYGGFDSPTVAYSVLVVAKVEK | |
| | GKSKKLKSVKELLGITIMERSSFEKNPIDFLEAKGYKEVK | |
| | KDLIIKLPKYSLFELENGRKRMLASAGELQKGNELALPSK | |
| | YVNFLYLASHYEKLKGSPEDNEQKQLFVEQHKHYLDEIIE | |
| | QISEFSKRVILADANLDKVLSAYNKHRDKPIREQAENIIH | |
| | LFTLTNLGAPAAFKYFDTTIDRKRYTSTKEVLDATLIHQS | |
| | ITGLYETRIDLSQLGGDSGGKRPAATKKAGQAKKKK | |
| 346 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | EZH2-dSpCas9 - |
| | QKHIQEWGPFDLVIGGSPCNDLSIVNPARKGLYEGTGRLF | No FLAG, P2A- |
| | FEFYRLLHDARPKEGDDRPFFWLFENVVAMGVSDKRDISR | mCherry |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEGQTGKKSEKGPVCWRKR | |
| | VKSEYMRLRQLKRFRRADEVKTMFSSNRQKILERTETLNQ | |
| | EWKQRRIQPVHIMTSVSSLRGTRECSVTSDLDFPAQVIPL | |
| | KTLNAVASVPIMYSWSPLQQNFMVEDETVLHNIPYMGDEV | |
| | LDQDGTFIEELIKNYDGKVHGDRECGFINDEIFVELVNAL | |
| | GQYNDDDDDDDGDDPDEREEKQKDLEDNRDDKETCPPRKF | |
| | PADKIFEAISSMFPDKGTAEELKEKYKELTEQQLPGALPP | |
| | ECTPNIDGPNAKSVQREQSLHSFHTLFCRRCFKYDCFLHP | |
| | FHATPNTYKRKNTETALDNKPCGPQCYQHLEGAKEFAAAL | |
| | TAERIKTPPKRPGGRRRGRLPNNSSRPSTPTISVLESKDT | |
| | DSDREAGTETGGENNDKEEEEKKDETSSSSEANSRCQTPI | |
| | KMKPNIEPPENVEWSGAEASMFRVLIGTYYDNFCAIARLI | |
| | GTKTCRQVYEFRVKESSIIAPVPTEDVDTPPRKKKRKHRL | |
| | WAAHCRKIQLKKDGSSNHVYNYQPCDHPRQPCDSSCPCVI | |
| | AQNFCEKFCQCSSECQNRFPGCRCKAQCNTKQCPCYLAVR | |
| | ECDPDLCLTCGAADHWDSKNVSCKNCSIQRGSKKHLLLAP | |
| | SDVAGWGIFIKDPVQKNEFISEYCGEIISQDEADRRGKVY | |
| | DKYMCSFLFNLNNDFVVDATRKGNKIRFANHSVNPNCYAK | |
| | VMMVNGDHRIGIFAKRAIQTGEELFFDYRYSQADALKYVG | |
| | IEREMEIPSTGGSGGSGGSGGSGGSGRPNPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGDSGGKRPAATKKAGQAKKKK | |
| 347 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-KOX1(2- |
| | VGTVKHEGQIKYVNDVRKITKKNIEEWGPFDLVIGGSPCN | 99) - No FLAG, |
| | DLSNVNPARKGLYEGTGRLFFEFYHLLNYTRPKEGDNRPF | P2A-mCherry |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASDAKSLTAWSRTLVTFKDVFVDFTREEWKLLDTA | |
| | QQILYRNVMLENYKNLVSLGYQLTKPDVILRLEKGEEPWL | |
| | VEREIHQETHPDSETAFEIKSSVPKKKRKV | |
| 348 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-KOX1(1- |
| | VGTVKHEGQIKYVNDVRKITKKNIEEWGPFDLVIGGSPCN | 72) - No FLAG, |
| | DLSNVNPARKGLYEGTGRLFFEFYHLLNYTRPKEGDNRPF | P2A-mCherry |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASMDAKSLTAWSRTLVTFKDVFVDFTREEWKLLDT | |
| | AQQIVYRNVMLENYKNLVSLGYQLTKPDVILRLEKGEEPP | |
| | KKKRKV | |
| 349 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-ZIM3 - |
| | VGTVKHEGQIKYVNDVRKITKKNIEEWGPFDLVIGGSPCN | No FLAG, P2A- |
| | DLSNVNPARKGLYEGTGRLFFEFYHLLNYTRPKEGDNRPF | mCherry |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRFNASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASNNSQGRVTFEDVTVNFTQGEWQRLNPEQRNLYR | |
| | DVMLENYSNLVSVGQGETTKPDVILRLEQGKEPWLEEEEV | |
| | LGSGRAEKNGDIGGQIWKPKDVKESLPKKKRKV | |
| 350 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-ZNF324 - |
| | VGTVKHEGQIKYVNDVRKITKKNIEEWGPFDLVIGGSPCN | No FLAG, P2A- |
| | DLSNVNPARKGLYEGTGRLFFEFYHLLNYTRPKEGDNRPF | mCherry |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASAFEDVAVYFSQEEWGLLDTAQRALYRRVMLDNF | |
| | ALVASLGLSTSRPRVVIQLERGEEPWVPSGTDTTLSRTTY | |
| | RRRNPGSWSLTEDRDVSGPKKKRKV | |
| 351 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-EZH2 - |
| | VGTVKHEGQIKYVNDVRKITKKNIEEWGPFDLVIGGSPCN | No FLAG, P2A- |
| | DLSNVNPARKGLYEGTGRLFFEFYHLLNYTRPKEGDNRPF | mCherry |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASSTGGSGGSGGSGGSGGSGRPGQTGKKSEKGPVC | |
| | WRKRVKSEYMRLRQLKRFRRADEVKTMFSSNRQKILERTE | |
| | TLNQEWKQRRIQPVHIMTSVSSLRGTRECSVTSDLDFPAQ | |
| | VIPLKTLNAVASVPIMYSWSPLQQNFMVEDETVLHNIPYM | |
| | GDEVLDQDGTFIEELIKNYDGKVHGDRECGFINDEIFVEL | |
| | VNALGQYNDDDDDDDGDDPDEREEKQKDLEDNRDDKETCP | |
| | PRKFPADKIFEAISSMFPDKGTAEELKEKYKELTEQQLPG | |
| | ALPPECTPNIDGPNAKSVQREQSLHSFHTLFCRRCFKYDC | |
| | FLHPFHATPNTYKRKNTETALDNKPCGPQCYQHLEGAKEF | |
| | AAALTAERIKTPPKRPGGRRRGRLPNNSSRPSTPTISVLE | |
| | SKDTDSDREAGTETGGENNDKEEEEKKDETSSSSEANSRC | |
| | QTPIKMKPNIEPPENVEWSGAEASMFRVLIGTYYDNFCAI | |
| | ARLIGTKTCRQVYEFRVKESSIIAPVPTEDVDTPPRKKKR | |
| | KHRLWAAHCRKIQLKKDGSSNHVYNYQPCDHPRQPCDSSC | |
| | PCVIAQNFCEKFCQCSSECQNRFPGCRCKAQCNTKQCPCY | |
| | LAVRECDPDLCLTCGAADHWDSKNVSCKNCSIQRGSKKHL | |
| | LLAPSDVAGWGIFIKDPVQKNEFISEYCGEIISQDEADRR | |
| | GKVYDKYMCSFLFNLNNDFVVDATRKGNKIRFANHSVNPN | |
| | CYAKVMMVNGDHRIGIFAKRAIQTGEELFFDYRYSQADAL | |
| | KYVGIEREMEIPPKKKRKV | |
| 352 | ATNFSLLKQAGDVEENPGP | P2A |
| 353 | MVSKGEEDNMAIIKEFMRFKVHMEGSVNGHEFEIEGEGEG | mCherry |
| | RPYEGTQTAKLKVTKGGPLPFAWDILSPQFMYGSKAYVKH | |
| | PADIPDYLKLSFPEGFKWERVMNFEDGGVVTVTQDSSLQD | |
| | GEFIYKVKLRGTNFPSDGPVMQKKTMGWEASSERMYPEDG | |
| | ALKGEIKQRLKLKDGGHYDAEVKTTYKAKKPVQLPGAYNV | |
| | NIKLDITSHNEDYTIVEQYERAEGRHSTGGMDELYK | |
| 354 | ATNFSLLKQAGDVEENPGPMVSKGEEDNMAIIKEFMRFKV | P2A-mCherry |
| | HMEGSVNGHEFEIEGEGEGRPYEGTQTAKLKVTKGGPLPF | |
| | AWDILSPQFMYGSKAYVKHPADIPDYLKLSFPEGFKWERV | |
| | MNFEDGGVVTVTQDSSLQDGEFIYKVKLRGTNFPSDGPVM | |
| | QKKTMGWEASSERMYPEDGALKGEIKQRLKLKDGGHYDAE | |
| | VKTTYKAKKPVQLPGAYNVNIKLDITSHNEDYTIVEQYER | |
| | AEGRHSTGGMDELYK | |
| 355 | DAKSLTAWSRTLVTFKDVFVDFTREEWKLLDTAQQILYRN | KRAB from |
| | VMLENYKNLVSLGYQLTKPDVILRLEKGEEPWLVEREIHQ | KOX1(2-99) |
| | ETHPDSETAFEIKSSV | |
| 356 | MDAKSLTAWSRTLVTFKDVFVDFTREEWKLLDTAQQIVYR | KRAB from |
| | NVMLENYKNLVSLGYQLTKPDVILRLEKGEEP | KOX1(1-72) |
| 357 | NNSQGRVTFEDVTVNFTQGEWQRLNPEQRNLYRDVMLENY | KRAB from ZIM3 |
| | SNLVSVGQGETTKPDVILRLEQGKEPWLEEEEVLGSGRAE | |
| | KNGDIGGQIWKPKDVKESL | |
| 358 | AFEDVAVYFSQEEWGLLDTAQRALYRRVMLDNFALVASLG | KRAB from |
| | LSTSRPRVVIQLERGEEPWVPSGTDTTLSRTTYRRRNPGS | ZNF324 |
| | WSLTEDRDVSG | |
| 359 | GQTGKKSEKGPVCWRKRVKSEYMRLRQLKRFRRADEVKTM | EZH2 domain |
| | FSSNRQKILERTETLNQEWKQRRIQPVHIMTSVSSLRGTR | |
| | ECSVTSDLDFPAQVIPLKTLNAVASVPIMYSWSPLQQNFM | |
| | VEDETVLHNIPYMGDEVLDQDGTFIEELIKNYDGKVHGDR | |
| | ECGFINDEIFVELVNALGQYNDDDDDDDGDDPDEREEKQK | |
| | DLEDNRDDKETCPPRKFPADKIFEAISSMFPDKGTAEELK | |
| | EKYKELTEQQLPGALPPECTPNIDGPNAKSVQREQSLHSF | |
| | HTLFCRRCFKYDCFLHPFHATPNTYKRKNTETALDNKPCG | |
| | PQCYQHLEGAKEFAAALTAERIKTPPKRPGGRRRGRLPNN | |
| | SSRPSTPTISVLESKDTDSDREAGTETGGENNDKEEEEKK | |
| | DETSSSSEANSRCQTPIKMKPNIEPPENVEWSGAEASMFR | |
| | VLIGTYYDNFCAIARLIGTKTCRQVYEFRVKESSIIAPVP | |
| | TEDVDTPPRKKKRKHRLWAAHCRKIQLKKDGSSNHVYNYQ | |
| | PCDHPRQPCDSSCPCVIAQNFCEKFCQCSSECQNRFPGCR | |
| | CKAQCNTKQCPCYLAVRECDPDLCLTCGAADHWDSKNVSC | |
| | KNCSIQRGSKKHLLLAPSDVAGWGIFIKDPVQKNEFISEY | |
| | CGEIISQDEADRRGKVYDKYMCSFLFNLNNDFVVDATRKG | |
| | NKIRFANHSVNPNCYAKVMMVNGDHRIGIFAKRAIQTGEE | |
| | LFFDYRYSQADALKYVGIEREMEIP | |
| 360 | VLRRRKDWNMRLQDFFTTDPDLEEFQEPPKLYPAIPAAKR | DNMT3B domain |
| | RPIRVLSLFDGIATGYLVLKELGIKVEKYIASEVCAESIA | |
| | VGTVKHEGQIKYVNDVRKITKKNIEEWGPFDLVIGGSPCN | |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | E | |
| 361 | GGPSSGAPPPSGGSPAGSPTSTEEGTSESATPESGPGTST | Linker - XTEN80 |
| | EPSEGSAPGSPAGSPTSTEEGTSTEPSEGSAPGTSTEPSE | |
| 362 | STGGSGGSGGSGGSGGSGRP | Linker - 20AA |
| 363 | VLRRRKDWNMRLQDFFTTDPDLEEFQEPPKLYPAIPAAKR | DNMT3B- |
| | RPIRVLSLFDGIATGYLVLKELGIKVEKYIASEVCAESIA | DNMT3L fusion |
| | VGTVKHEGQIKYVNDVRKITKKNIEEWGPFDLVIGGSPCN | domain |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPL | |
| 364 | DYKDHDGDYKDHDIDYKDDDDKDKKYSIGLAIGTNSVGWA | FLAG tag (AA) |
| 365 | VITDEYKVPSKKFKVLGNTDRHSIKKNLIGALLFDSGETA | dSpCas9-KOX1(2- |
| | EATRLKRTARRRYTRRKNRICYLQEIFSNEMAKVDDSFFH | 99) - minimal |
| | RLEESFLVEEDKKHERHPIFGNIVDEVAYHEKYPTIYHLR | |
| | KKLVDSTDKADLRLIYLALAHMIKFRGHFLIEGDLNPDNS | |
| | DVDKLFIQLVQTYNQLFEENPINASGVDAKAILSARLSKS | |
| | RRLENLIAQLPGEKKNGLFGNLIALSLGLTPNFKSNFDLA | |
| | EDAKLQLSKDTYDDDLDNLLAQIGDQYADLFLAAKNLSDA | |
| | ILLSDILRVNTEITKAPLSASMIKRYDEHHQDLTLLKALV | |
| | RQQLPEKYKEIFFDQSKNGYAGYIDGGASQEEFYKFIKPI | |
| | LEKMDGTEELLVKLNREDLLRKQRTFDNGSIPHQIHLGEL | |
| | HAILRRQEDFYPFLKDNREKIEKILTFRIPYYVGPLARGN | |
| | SRFAWMTRKSEETITPWNFEEVVDKGASAQSFIERMTNFD | |
| | KNLPNEKVLPKHSLLYEYFTVYNELTKVKYVTEGMRKPAF | |
| | LSGEQKKAIVDLLFKTNRKVTVKQLKEDYFKKIECFDSVE | |
| | ISGVEDRFNASLGTYHDLLKIIKDKDFLDNEENEDILEDI | |
| | VLTLTLFEDREMIEERLKTYAHLFDDKVMKQLKRRRYTGW | |
| | GRLSRKLINGIRDKQSGKTILDFLKSDGFANRNFMQLIHD | |
| | DSLTFKEDIQKAQVSGQGDSLHEHIANLAGSPAIKKGILQ | |
| | TVKVVDELVKVMGRHKPENIVIEMARENQTTQKGQKNSRE | |
| | RMKRIEEGIKELGSQILKEHPVENTQLQNEKLYLYYLQNG | |
| | RDMYVDQELDINRLSDYDVDAIVPQSFLKDDSIDNKVLTR | |
| | SDKNRGKSDNVPSEEVVKKMKNYWRQLLNAKLITQRKFDN | |
| | LTKAERGGLSELDKAGFIKRQLVETRQITKHVAQILDSRM | |
| | NTKYDENDKLIREVKVITLKSKLVSDFRKDFQFYKVREIN | |
| | NYHHAHDAYLNAVVGTALIKKYPKLESEFVYGDYKVYDVR | |
| | KMIAKSEQEIGKATAKYFFYSNIMNFFKTEITLANGEIRK | |
| | RPLIETNGETGEIVWDKGRDFATVRKVLSMPQVNIVKKTE | |
| | VQTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAGE | |
| | LQKGNELALPSKYVNFLYLASHYEKLKGSPEDNEQKQLFV | |
| | EQHKHYLDEIIEQISEFSKRVILADANLDKVLSAYNKHRD | |
| | KPIREQAENIIHLFTLTNLGAPAAFKYFDTTIDRKRYTST | |
| | KEVLDATLIHQSITGLYETRIDLSQLGGDSGGKRPAATKK | |
| | AGQAKKKKASDAKSLTAWSRTLVTFKDVFVDFTREEWKLL | |
| | DTAQQILYRNVMLENYKNLVSLGYQLTKPDVILRLEKGEE | |
| | PWLVEREIHQETHPDSETAFEIKSSV | |
| 366 | DKKYSIGLAIGTNSVGWAVITDEYKVPSKKFKVLGNTDRH | dSpCas9-KOX1(1- |
| | SIKKNLIGALLFDSGETAEATRLKRTARRRYTRRKNRICY | 72) - minimal |
| | LQEIFSNEMAKVDDSFFHRLEESFLVEEDKKHERHPIFGN | |
| | IVDEVAYHEKYPTIYHLRKKLVDSTDKADLRLIYLALAHM | |
| | IKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLAQ | |
| | IGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIE | |
| | KILTFRIPYYVGPLARGNSRFAWMTRKSEETITPWNFEEV | |
| | VDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKII | |
| | KDKDFLDNEENEDILEDIVLTLTLFEDREMIEERLKTYAH | |
| | LFDDKVMKQLKRRRYTGWGRLSRKLINGIRDKQSGKTILD | |
| | FLKSDGFANRNFMQLIHDDSLTFKEDIQKAQVSGQGDSLH | |
| | EHIANLAGSPAIKKGILQTVKVVDELVKVMGRHKPENIVI | |
| | EMARENQTTQKGQKNSRERMKRIEEGIKELGSQILKEHPV | |
| | ENTQLQNEKLYLYYLQNGRDMYVDQELDINRLSDYDVDAI | |
| | VPQSFLKDDSIDNKVLTRSDKNRGKSDNVPSEEVVKKMKN | |
| | YWRQLLNAKLITQRKFDNLTKAERGGLSELDKAGFIKRQL | |
| | VETRQITKHVAQILDSRMNTKYDENDKLIREVKVITLKSK | |
| | LVSDFRKDFQFYKVREINNYHHAHDAYLNAVVGTALIKKY | |
| | PKLESEFVYGDYKVYDVRKMIAKSEQEIGKATAKYFFYSN | |
| | IMNFFKTEITLANGEIRKRPLIETNGETGEIVWDKGRDFA | |
| | TVRKVLSMPQVNIVKKTEVQTGGFSKESILPKRNSDKLIA | |
| | RKKDWDPKKYGGFDSPTVAYSVLVVAKVEKGKSKKLKSVK | |
| | ELLGITIMERSSFEKNPIDFLEAKGYKEVKKDLIIKLPKY | |
| | SLFELENGRKRMLASAGELQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKRYTSTKEVLDATLIHQSITGLYETRID | |
| | LSQLGGDSGGKRPAATKKAGQAKKKKASMDAKSLTAWSRT | |
| | LVTFKDVFVDFTREEWKLLDTAQQIVYRNVMLENYKNLVS | |
| | LGYQLTKPDVILRLEKGEEP | |
| 367 | DKKYSIGLAIGTNSVGWAVITDEYKVPSKKFKVLGNTDRH | dSpCas9-ZIM3 - |
| | SIKKNLIGALLFDSGETAEATRLKRTARRRYTRRKNRICY | minimal |
| | LQEIFSNEMAKVDDSFFHRLEESFLVEEDKKHERHPIFGN | |
| | IVDEVAYHEKYPTIYHLRKKLVDSTDKADLRLIYLALAHM | |
| | IKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLAQ | |
| | IGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIE | |
| | KILTFRIPYYVGPLARGNSRFAWMTRKSEETITPWNFEEV | |
| | VDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKII | |
| | KDKDFLDNEENEDILEDIVLTLTLFEDREMIEERLKTYAH | |
| | LFDDKVMKQLKRRRYTGWGRLSRKLINGIRDKQSGKTILD | |
| | FLKSDGFANRNFMQLIHDDSLTFKEDIQKAQVSGQGDSLH | |
| | EHIANLAGSPAIKKGILQTVKVVDELVKVMGRHKPENIVI | |
| | EMARENQTTQKGQKNSRERMKRIEEGIKELGSQILKEHPV | |
| | ENTQLQNEKLYLYYLQNGRDMYVDQELDINRLSDYDVDAI | |
| | VPQSFLKDDSIDNKVLTRSDKNRGKSDNVPSEEVVKKMKN | |
| | YWRQLLNAKLITQRKFDNLTKAERGGLSELDKAGFIKRQL | |
| | VETRQITKHVAQILDSRMNTKYDENDKLIREVKVITLKSK | |
| | LVSDFRKDFQFYKVREINNYHHAHDAYLNAVVGTALIKKY | |
| | PKLESEFVYGDYKVYDVRKMIAKSEQEIGKATAKYFFYSN | |
| | IMNFFKTEITLANGEIRKRPLIETNGETGEIVWDKGRDFA | |
| | TVRKVLSMPQVNIVKKTEVQTGGFSKESILPKRNSDKLIA | |
| | RKKDWDPKKYGGFDSPTVAYSVLVVAKVEKGKSKKLKSVK | |
| | ELLGITIMERSSFEKNPIDFLEAKGYKEVKKDLIIKLPKY | |
| | SLFELENGRKRMLASAGELQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKRYTSTKEVLDATLIHQSITGLYETRID | |
| | LSQLGGDSGGKRPAATKKAGQAKKKKASNNSQGRVTFEDV | |
| | TVNFTQGEWQRLNPEQRNLYRDVMLENYSNLVSVGQGETT | |
| | KPDVILRLEQGKEPWLEEEEVLGSGRAEKNGDIGGQIWKP | |
| | KDVKESL | |
| 368 | DKKYSIGLAIGTNSVGWAVITDEYKVPSKKFKVLGNTDRH | dSpCas9-ZNF324 - |
| | SIKKNLIGALLFDSGETAEATRLKRTARRRYTRRKNRICY | minimal |
| | LQEIFSNEMAKVDDSFFHRLEESFLVEEDKKHERHPIFGN | |
| | IVDEVAYHEKYPTIYHLRKKLVDSTDKADLRLIYLALAHM | |
| | IKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLAQ | |
| | IGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIE | |
| | KILTFRIPYYVGPLARGNSRFAWMTRKSEETITPWNFEEV | |
| | VDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKII | |
| | KDKDFLDNEENEDILEDIVLTLTLFEDREMIEERLKTYAH | |
| | LFDDKVMKQLKRRRYTGWGRLSRKLINGIRDKQSGKTILD | |
| | FLKSDGFANRNFMQLIHDDSLTFKEDIQKAQVSGQGDSLH | |
| | EHIANLAGSPAIKKGILQTVKVVDELVKVMGRHKPENIVI | |
| | EMARENQTTQKGQKNSRERMKRIEEGIKELGSQILKEHPV | |
| | ENTQLQNEKLYLYYLQNGRDMYVDQELDINRLSDYDVDAI | |
| | VPQSFLKDDSIDNKVLTRSDKNRGKSDNVPSEEVVKKMKN | |
| | YWRQLLNAKLITQRKFDNLTKAERGGLSELDKAGFIKRQL | |
| | VETRQITKHVAQILDSRMNTKYDENDKLIREVKVITLKSK | |
| | LVSDFRKDFQFYKVREINNYHHAHDAYLNAVVGTALIKKY | |
| | PKLESEFVYGDYKVYDVRKMIAKSEQEIGKATAKYFFYSN | |
| | IMNFFKTEITLANGEIRKRPLIETNGETGEIVWDKGRDFA | |
| | TVRKVLSMPQVNIVKKTEVQTGGFSKESILPKRNSDKLIA | |
| | RKKDWDPKKYGGFDSPTVAYSVLVVAKVEKGKSKKLKSVK | |
| | ELLGITIMERSSFEKNPIDFLEAKGYKEVKKDLIIKLPKY | |
| | SLFELENGRKRMLASAGELQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKRYTSTKEVLDATLIHQSITGLYETRID | |
| | LSQLGGDSGGKRPAATKKAGQAKKKKASAFEDVAVYFSQE | |
| | EWGLLDTAQRALYRRVMLDNFALVASLGLSTSRPRVVIQL | |
| | ERGEEPWVPSGTDTTLSRTTYRRRNPGSWSLTEDRDVSG | |
| 369 | DKKYSIGLAIGTNSVGWAVITDEYKVPSKKFKVLGNTDRH | dSpCas9-EZH2 - |
| | SIKKNLIGALLFDSGETAEATRLKRTARRRYTRRKNRICY | minimal |
| | LQEIFSNEMAKVDDSFFHRLEESFLVEEDKKHERHPIFGN | |
| | IVDEVAYHEKYPTIYHLRKKLVDSTDKADLRLIYLALAHM | |
| | IKFRGHFLIEGDLNPDNSDVDKLFIQLVQTYNQLFEENPI | |
| | NASGVDAKAILSARLSKSRRLENLIAQLPGEKKNGLFGNL | |
| | IALSLGLTPNFKSNFDLAEDAKLQLSKDTYDDDLDNLLAQ | |
| | IGDQYADLFLAAKNLSDAILLSDILRVNTEITKAPLSASM | |
| | IKRYDEHHQDLTLLKALVRQQLPEKYKEIFFDQSKNGYAG | |
| | YIDGGASQEEFYKFIKPILEKMDGTEELLVKLNREDLLRK | |
| | QRTFDNGSIPHQIHLGELHAILRRQEDFYPFLKDNREKIE | |
| | KILTFRIPYYVGPLARGNSRFAWMTRKSEETITPWNFEEV | |
| | VDKGASAQSFIERMTNFDKNLPNEKVLPKHSLLYEYFTVY | |
| | NELTKVKYVTEGMRKPAFLSGEQKKAIVDLLFKTNRKVTV | |
| | KQLKEDYFKKIECFDSVEISGVEDRFNASLGTYHDLLKII | |
| | KDKDFLDNEENEDILEDIVLTLTLFEDREMIEERLKTYAH | |
| | LFDDKVMKQLKRRRYTGWGRLSRKLINGIRDKQSGKTILD | |
| | FLKSDGFANRNFMQLIHDDSLTFKEDIQKAQVSGQGDSLH | |
| | EHIANLAGSPAIKKGILQTVKVVDELVKVMGRHKPENIVI | |
| | EMARENQTTQKGQKNSRERMKRIEEGIKELGSQILKEHPV | |
| | ENTQLQNEKLYLYYLQNGRDMYVDQELDINRLSDYDVDAI | |
| | VPQSFLKDDSIDNKVLTRSDKNRGKSDNVPSEEVVKKMKN | |
| | YWRQLLNAKLITQRKFDNLTKAERGGLSELDKAGFIKRQL | |
| | VETRQITKHVAQILDSRMNTKYDENDKLIREVKVITLKSK | |
| | LVSDFRKDFQFYKVREINNYHHAHDAYLNAVVGTALIKKY | |
| | PKLESEFVYGDYKVYDVRKMIAKSEQEIGKATAKYFFYSN | |
| | IMNFFKTEITLANGEIRKRPLIETNGETGEIVWDKGRDFA | |
| | TVRKVLSMPQVNIVKKTEVQTGGFSKESILPKRNSDKLIA | |
| | RKKDWDPKKYGGFDSPTVAYSVLVVAKVEKGKSKKLKSVK | |
| | ELLGITIMERSSFEKNPIDFLEAKGYKEVKKDLIIKLPKY | |
| | SLFELENGRKRMLASAGELQKGNELALPSKYVNFLYLASH | |
| | YEKLKGSPEDNEQKQLFVEQHKHYLDEIIEQISEFSKRVI | |
| | LADANLDKVLSAYNKHRDKPIREQAENIIHLFTLTNLGAP | |
| | AAFKYFDTTIDRKRYTSTKEVLDATLIHQSITGLYETRID | |
| | LSQLGGDSGGKRPAATKKAGQAKKKKASSTGGSGGSGGSG | |
| | GSGGSGRPGQTGKKSEKGPVCWRKRVKSEYMRLRQLKRFR | |
| | RADEVKTMFSSNRQKILERTETLNQEWKQRRIQPVHIMTS | |
| | VSSLRGTRECSVTSDLDFPAQVIPLKTLNAVASVPIMYSW | |
| | SPLQQNFMVEDETVLHNIPYMGDEVLDQDGTFIEELIKNY | |
| | DGKVHGDRECGFINDEIFVELVNALGQYNDDDDDDDGDDP | |
| | DEREEKQKDLEDNRDDKETCPPRKFPADKIFEAISSMFPD | |
| | KGTAEELKEKYKELTEQQLPGALPPECTPNIDGPNAKSVQ | |
| | REQSLHSFHTLFCRRCFKYDCFLHPFHATPNTYKRKNTET | |
| | ALDNKPCGPQCYQHLEGAKEFAAALTAERIKTPPKRPGGR | |
| | RRGRLPNNSSRPSTPTISVLESKDTDSDREAGTETGGENN | |
| | DKEEEEKKDETSSSSEANSRCQTPIKMKPNIEPPENVEWS | |
| | GAEASMFRVLIGTYYDNFCAIARLIGTKTCRQVYEFRVKE | |
| | SSIIAPVPTEDVDTPPRKKKRKHRLWAAHCRKIQLKKDGS | |
| | SNHVYNYQPCDHPRQPCDSSCPCVIAQNFCEKFCQCSSEC | |
| | QNRFPGCRCKAQCNTKQCPCYLAVRECDPDLCLTCGAADH | |
| | WDSKNVSCKNCSIQRGSKKHLLLAPSDVAGWGIFIKDPVQ | |
| | KNEFISEYCGEIISQDEADRRGKVYDKYMCSFLFNLNNDF | |
| | VVDATRKGNKIRFANHSVNPNCYAKVMMVNGDHRIGIFAK | |
| | RAIQTGEELFFDYRYSQADALKYVGIEREMEIP | |
| 370 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-KOX1(2- |
| | QKHIQEWGPFDLVIGGSPCNDLSIVNPARKGLYEGTGRLF | 99) - minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASDAKSLTAWSRTLV | |
| | TFKDVFVDFTREEWKLLDTAQQILYRNVMLENYKNLVSLG | |
| | YQLTKPDVILRLEKGEEPWLVEREIHQETHPDSETAFEIK | |
| | SSV | |
| 371 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-KOX1(1- |
| | QKHIQEWGPFDLVIGGSPCNDLSIVNPARKGLYEGTGRLF | 72) - minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASMDAKSLTAWSRTL | |
| | VTFKDVFVDFTREEWKLLDTAQQIVYRNVMLENYKNLVSL | |
| | GYQLTKPDVILRLEKGEEP | |
| 372 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-ZIM3 - |
| | QKHIQEWGPFDLVIGGSPCNDLSIVNPARKGLYEGTGRLF | minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASNNSQGRVTFEDVT | |
| | VNFTQGEWQRLNPEQRNLYRDVMLENYSNLVSVGQGETTK | |
| | PDVILRLEQGKEPWLEEEEVLGSGRAEKNGDIGGQIWKPK | |
| | DVKESL | |
| 373 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-ZNF324 - |
| | QKHIQEWGPFDLVIGGSPCNDLSIVNPARKGLYEGTGRLF | minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASAFEDVAVYFSQEE | |
| | WGLLDTAQRALYRRVMLDNFALVASLGLSTSRPRVVIQLE | |
| | RGEEPWVPSGTDTTLSRTTYRRRNPGSWSLTEDRDVSG | |
| 374 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | dSpCas9-EZH2 - |
| | QKHIQEWGPFDLVIGGSPCNDLSIVNPARKGLYEGTGRLF | minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENPKKKRKVGIHGVPAAD | |
| | KKYSIGLAIGTNSVGWAVITDEYKVPSKKFKVLGNTDRHS | |
| | IKKNLIGALLFDSGETAEATRLKRTARRRYTRRKNRICYL | |
| | QEIFSNEMAKVDDSFFHRLEESFLVEEDKKHERHPIFGNI | |
| | VDEVAYHEKYPTIYHLRKKLVDSTDKADLRLIYLALAHMI | |
| | KFRGHFLIEGDLNPDNSDVDKLFIQLVQTYNQLFEENPIN | |
| | ASGVDAKAILSARLSKSRRLENLIAQLPGEKKNGLFGNLI | |
| | ALSLGLTPNFKSNFDLAEDAKLQLSKDTYDDDLDNLLAQI | |
| | GDQYADLFLAAKNLSDAILLSDILRVNTEITKAPLSASMI | |
| | KRYDEHHQDLTLLKALVRQQLPEKYKEIFFDQSKNGYAGY | |
| | IDGGASQEEFYKFIKPILEKMDGTEELLVKLNREDLLRKQ | |
| | RTFDNGSIPHQIHLGELHAILRRQEDFYPFLKDNREKIEK | |
| | ILTFRIPYYVGPLARGNSRFAWMTRKSEETITPWNFEEVV | |
| | DKGASAQSFIERMTNFDKNLPNEKVLPKHSLLYEYFTVYN | |
| | ELTKVKYVTEGMRKPAFLSGEQKKAIVDLLFKTNRKVTVK | |
| | QLKEDYFKKIECFDSVEISGVEDRFNASLGTYHDLLKIIK | |
| | DKDFLDNEENEDILEDIVLTLTLFEDREMIEERLKTYAHL | |
| | FDDKVMKQLKRRRYTGWGRLSRKLINGIRDKQSGKTILDF | |
| | LKSDGFANRNFMQLIHDDSLTFKEDIQKAQVSGQGDSLHE | |
| | HIANLAGSPAIKKGILQTVKVVDELVKVMGRHKPENIVIE | |
| | MARENQTTQKGQKNSRERMKRIEEGIKELGSQILKEHPVE | |
| | NTQLQNEKLYLYYLQNGRDMYVDQELDINRLSDYDVDAIV | |
| | PQSFLKDDSIDNKVLTRSDKNRGKSDNVPSEEVVKKMKNY | |
| | WRQLLNAKLITQRKFDNLTKAERGGLSELDKAGFIKRQLV | |
| | ETRQITKHVAQILDSRMNTKYDENDKLIREVKVITLKSKL | |
| | VSDFRKDFQFYKVREINNYHHAHDAYLNAVVGTALIKKYP | |
| | KLESEFVYGDYKVYDVRKMIAKSEQEIGKATAKYFFYSNI | |
| | MNFFKTEITLANGEIRKRPLIETNGETGEIVWDKGRDFAT | |
| | VRKVLSMPQVNIVKKTEVQTGGFSKESILPKRNSDKLIAR | |
| | KKDWDPKKYGGFDSPTVAYSVLVVAKVEKGKSKKLKSVKE | |
| | LLGITIMERSSFEKNPIDFLEAKGYKEVKKDLIIKLPKYS | |
| | LFELENGRKRMLASAGELQKGNELALPSKYVNFLYLASHY | |
| | EKLKGSPEDNEQKQLFVEQHKHYLDEIIEQISEFSKRVIL | |
| | ADANLDKVLSAYNKHRDKPIREQAENIIHLFTLTNLGAPA | |
| | AFKYFDTTIDRKRYTSTKEVLDATLIHQSITGLYETRIDL | |
| | SQLGGDSGGKRPAATKKAGQAKKKKASSTGGSGGSGGSGG | |
| | SGGSGRPGQTGKKSEKGPVCWRKRVKSEYMRLRQLKRFRR | |
| | ADEVKTMFSSNRQKILERTETLNQEWKQRRIQPVHIMTSV | |
| | SSLRGTRECSVTSDLDFPAQVIPLKTLNAVASVPIMYSWS | |
| | PLQQNFMVEDETVLHNIPYMGDEVLDQDGTFIEELIKNYD | |
| | GKVHGDRECGFINDEIFVELVNALGQYNDDDDDDDGDDPD | |
| | EREEKQKDLEDNRDDKETCPPRKFPADKIFEAISSMFPDK | |
| | GTAEELKEKYKELTEQQLPGALPPECTPNIDGPNAKSVQR | |
| | EQSLHSFHTLFCRRCFKYDCFLHPFHATPNTYKRKNTETA | |
| | LDNKPCGPQCYQHLEGAKEFAAALTAERIKTPPKRPGGRR | |
| | RGRLPNNSSRPSTPTISVLESKDTDSDREAGTETGGENND | |
| | KEEEEKKDETSSSSEANSRCQTPIKMKPNIEPPENVEWSG | |
| | AEASMFRVLIGTYYDNFCAIARLIGTKTCRQVYEFRVKES | |
| | SIIAPVPTEDVDTPPRKKKRKHRLWAAHCRKIQLKKDGSS | |
| | NHVYNYQPCDHPRQPCDSSCPCVIAQNFCEKFCQCSSECQ | |
| | NRFPGCRCKAQCNTKQCPCYLAVRECDPDLCLTCGAADHW | |
| | DSKNVSCKNCSIQRGSKKHLLLAPSDVAGWGIFIKDPVQK | |
| | NEFISEYCGEIISQDEADRRGKVYDKYMCSFLFNLNNDFV | |
| | VDATRKGNKIRFANHSVNPNCYAKVMMVNGDHRIGIFAKR | |
| | AIQTGEELFFDYRYSQADALKYVGIEREMEIP | |
| 375 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | KOX1(2-99)- |
| | QKHIQEWGPFDLVIGGSPCNDLSIVNPARKGLYEGTGRLF | dSpCas9 - minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEDAKSLTAWSRTLVTFKD | |
| | VFVDFTREEWKLLDTAQQILYRNVMLENYKNLVSLGYQLT | |
| | KPDVILRLEKGEEPWLVEREIHQETHPDSETAFEIKSSVN | |
| | PKKKRKVGIHGVPAADKKYSIGLAIGTNSVGWAVITDEYK | |
| | VPSKKFKVLGNTDRHSIKKNLIGALLFDSGETAEATRLKR | |
| | TARRRYTRRKNRICYLQEIFSNEMAKVDDSFFHRLEESFL | |
| | VEEDKKHERHPIFGNIVDEVAYHEKYPTIYHLRKKLVDST | |
| | DKADLRLIYLALAHMIKFRGHFLIEGDLNPDNSDVDKLFI | |
| | QLVQTYNQLFEENPINASGVDAKAILSARLSKSRRLENLI | |
| | AQLPGEKKNGLFGNLIALSLGLTPNFKSNFDLAEDAKLQL | |
| | SKDTYDDDLDNLLAQIGDQYADLFLAAKNLSDAILLSDIL | |
| | RVNTEITKAPLSASMIKRYDEHHQDLTLLKALVRQQLPEK | |
| | YKEIFFDQSKNGYAGYIDGGASQEEFYKFIKPILEKMDGT | |
| | EELLVKLNREDLLRKQRTFDNGSIPHQIHLGELHAILRRQ | |
| | EDFYPFLKDNREKIEKILTFRIPYYVGPLARGNSRFAWMT | |
| | RKSEETITPWNFEEVVDKGASAQSFIERMTNFDKNLPNEK | |
| | VLPKHSLLYEYFTVYNELTKVKYVTEGMRKPAFLSGEQKK | |
| | AIVDLLFKTNRKVTVKQLKEDYFKKIECFDSVEISGVEDR | |
| | FNASLGTYHDLLKIIKDKDFLDNEENEDILEDIVLTLTLF | |
| | EDREMIEERLKTYAHLFDDKVMKQLKRRRYTGWGRLSRKL | |
| | INGIRDKQSGKTILDFLKSDGFANRNFMQLIHDDSLTFKE | |
| | DIQKAQVSGQGDSLHEHIANLAGSPAIKKGILQTVKVVDE | |
| | LVKVMGRHKPENIVIEMARENQTTQKGQKNSRERMKRIEE | |
| | GIKELGSQILKEHPVENTQLQNEKLYLYYLQNGRDMYVDQ | |
| | ELDINRLSDYDVDAIVPQSFLKDDSIDNKVLTRSDKNRGK | |
| | SDNVPSEEVVKKMKNYWRQLLNAKLITQRKFDNLTKAERG | |
| | GLSELDKAGFIKRQLVETRQITKHVAQILDSRMNTKYDEN | |
| | DKLIREVKVITLKSKLVSDFRKDFQFYKVREINNYHHAHD | |
| | AYLNAVVGTALIKKYPKLESEFVYGDYKVYDVRKMIAKSE | |
| | QEIGKATAKYFFYSNIMNFFKTEITLANGEIRKRPLIETN | |
| | GETGEIVWDKGRDFATVRKVLSMPQVNIVKKTEVQTGGFS | |
| | KESILPKRNSDKLIARKKDWDPKKYGGFDSPTVAYSVLVV | |
| | AKVEKGKSKKLKSVKELLGITIMERSSFEKNPIDFLEAKG | |
| | YKEVKKDLIIKLPKYSLFELENGRKRMLASAGELQKGNEL | |
| | ALPSKYVNFLYLASHYEKLKGSPEDNEQKQLFVEQHKHYL | |
| | DEIIEQISEFSKRVILADANLDKVLSAYNKHRDKPIREQA | |
| | ENIIHLFTLTNLGAPAAFKYFDTTIDRKRYTSTKEVLDAT | |
| | LIHQSITGLYETRIDLSQLGGD | |
| 376 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | KOX1(1-72)- |
| | QKHIQEWGPFDLVIGGSPCNDLSIVNPARKGLYEGTGRLF | dSpCas9 - minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEMDAKSLTAWSRTLVTFK | |
| | DVFVDFTREEWKLLDTAQQIVYRNVMLENYKNLVSLGYQL | |
| | TKPDVILRLEKGEEPNPKKKRKVGIHGVPAADKKYSIGLA | |
| | IGTNSVGWAVITDEYKVPSKKFKVLGNTDRHSIKKNLIGA | |
| | LLFDSGETAEATRLKRTARRRYTRRKNRICYLQEIFSNEM | |
| | AKVDDSFFHRLEESFLVEEDKKHERHPIFGNIVDEVAYHE | |
| | KYPTIYHLRKKLVDSTDKADLRLIYLALAHMIKFRGHFLI | |
| | EGDLNPDNSDVDKLFIQLVQTYNQLFEENPINASGVDAKA | |
| | ILSARLSKSRRLENLIAQLPGEKKNGLFGNLIALSLGLTP | |
| | NFKSNFDLAEDAKLQLSKDTYDDDLDNLLAQIGDQYADLF | |
| | LAAKNLSDAILLSDILRVNTEITKAPLSASMIKRYDEHHQ | |
| | DLTLLKALVRQQLPEKYKEIFFDQSKNGYAGYIDGGASQE | |
| | EFYKFIKPILEKMDGTEELLVKLNREDLLRKQRTFDNGSI | |
| | PHQIHLGELHAILRRQEDFYPFLKDNREKIEKILTFRIPY | |
| | YVGPLARGNSRFAWMTRKSEETITPWNFEEVVDKGASAQS | |
| | FIERMTNFDKNLPNEKVLPKHSLLYEYFTVYNELTKVKYV | |
| | TEGMRKPAFLSGEQKKAIVDLLFKTNRKVTVKQLKEDYFK | |
| | KIECFDSVEISGVEDRFNASLGTYHDLLKIIKDKDFLDNE | |
| | ENEDILEDIVLTLTLFEDREMIEERLKTYAHLFDDKVMKQ | |
| | LKRRRYTGWGRLSRKLINGIRDKQSGKTILDFLKSDGFAN | |
| | RNFMQLIHDDSLTFKEDIQKAQVSGQGDSLHEHIANLAGS | |
| | PAIKKGILQTVKVVDELVKVMGRHKPENIVIEMARENQTT | |
| | QKGQKNSRERMKRIEEGIKELGSQILKEHPVENTQLQNEK | |
| | LYLYYLQNGRDMYVDQELDINRLSDYDVDAIVPQSFLKDD | |
| | SIDNKVLTRSDKNRGKSDNVPSEEVVKKMKNYWRQLLNAK | |
| | LITQRKFDNLTKAERGGLSELDKAGFIKRQLVETRQITKH | |
| | VAQILDSRMNTKYDENDKLIREVKVITLKSKLVSDFRKDF | |
| | QFYKVREINNYHHAHDAYLNAVVGTALIKKYPKLESEFVY | |
| | GDYKVYDVRKMIAKSEQEIGKATAKYFFYSNIMNFFKTEI | |
| | TLANGEIRKRPLIETNGETGEIVWDKGRDFATVRKVLSMP | |
| | QVNIVKKTEVQTGGFSKESILPKRNSDKLIARKKDWDPKK | |
| | YGGFDSPTVAYSVLVVAKVEKGKSKKLKSVKELLGITIME | |
| | RSSFEKNPIDFLEAKGYKEVKKDLIIKLPKYSLFELENGR | |
| | KRMLASAGELQKGNELALPSKYVNFLYLASHYEKLKGSPE | |
| | DNEQKQLFVEQHKHYLDEIIEQISEFSKRVILADANLDKV | |
| | LSAYNKHRDKPIREQAENIIHLFTLTNLGAPAAFKYFDTT | |
| | IDRKRYTSTKEVLDATLIHQSITGLYETRIDLSQLGGD | |
| 377 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | ZIM3-dSpCas9 - |
| | QKHIQEWGPFDLVIGGSPCNDLSIVNPARKGLYEGTGRLF | minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSENNSQGRVTFEDVTVNFT | |
| | QGEWQRLNPEQRNLYRDVMLENYSNLVSVGQGETTKPDVI | |
| | LRLEQGKEPWLEEEEVLGSGRAEKNGDIGGQIWKPKDVKE | |
| | SLNPKKKRKVGIHGVPAADKKYSIGLAIGTNSVGWAVITD | |
| | EYKVPSKKFKVLGNTDRHSIKKNLIGALLFDSGETAEATR | |
| | LKRTARRRYTRRKNRICYLQEIFSNEMAKVDDSFFHRLEE | |
| | SFLVEEDKKHERHPIFGNIVDEVAYHEKYPTIYHLRKKLV | |
| | DSTDKADLRLIYLALAHMIKFRGHFLIEGDLNPDNSDVDK | |
| | LFIQLVQTYNQLFEENPINASGVDAKAILSARLSKSRRLE | |
| | NLIAQLPGEKKNGLFGNLIALSLGLTPNFKSNFDLAEDAK | |
| | LQLSKDTYDDDLDNLLAQIGDQYADLFLAAKNLSDAILLS | |
| | DILRVNTEITKAPLSASMIKRYDEHHQDLTLLKALVRQQL | |
| | PEKYKEIFFDQSKNGYAGYIDGGASQEEFYKFIKPILEKM | |
| | DGTEELLVKLNREDLLRKQRTFDNGSIPHQIHLGELHAIL | |
| | RRQEDFYPFLKDNREKIEKILTFRIPYYVGPLARGNSRFA | |
| | WMTRKSEETITPWNFEEVVDKGASAQSFIERMTNFDKNLP | |
| | NEKVLPKHSLLYEYFTVYNELTKVKYVTEGMRKPAFLSGE | |
| | QKKAIVDLLFKTNRKVTVKQLKEDYFKKIECFDSVEISGV | |
| | EDRFNASLGTYHDLLKIIKDKDFLDNEENEDILEDIVLTL | |
| | TLFEDREMIEERLKTYAHLFDDKVMKQLKRRRYTGWGRLS | |
| | RKLINGIRDKQSGKTILDFLKSDGFANRNFMQLIHDDSLT | |
| | FKEDIQKAQVSGQGDSLHEHIANLAGSPAIKKGILQTVKV | |
| | VDELVKVMGRHKPENIVIEMARENQTTQKGQKNSRERMKR | |
| | IEEGIKELGSQILKEHPVENTQLQNEKLYLYYLQNGRDMY | |
| | VDQELDINRLSDYDVDAIVPQSFLKDDSIDNKVLTRSDKN | |
| | RGKSDNVPSEEVVKKMKNYWRQLLNAKLITQRKFDNLTKA | |
| | ERGGLSELDKAGFIKRQLVETRQITKHVAQILDSRMNTKY | |
| | DENDKLIREVKVITLKSKLVSDFRKDFQFYKVREINNYHH | |
| | AHDAYLNAVVGTALIKKYPKLESEFVYGDYKVYDVRKMIA | |
| | KSEQEIGKATAKYFFYSNIMNFFKTEITLANGEIRKRPLI | |
| | ETNGETGEIVWDKGRDFATVRKVLSMPQVNIVKKTEVQTG | |
| | GFSKESILPKRNSDKLIARKKDWDPKKYGGFDSPTVAYSV | |
| | LVVAKVEKGKSKKLKSVKELLGITIMERSSFEKNPIDFLE | |
| | AKGYKEVKKDLIIKLPKYSLFELENGRKRMLASAGELQKG | |
| | NELALPSKYVNFLYLASHYEKLKGSPEDNEQKQLFVEQHK | |
| | HYLDEIIEQISEFSKRVILADANLDKVLSAYNKHRDKPIR | |
| | EQAENIIHLFTLTNLGAPAAFKYFDTTIDRKRYTSTKEVL | |
| | DATLIHQSITGLYETRIDLSQLGGD | |
| 378 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | ZNF324-dSpCas9 - |
| | QKHIQEWGPFDLVIGGSPCNDLSIVNPARKGLYEGTGRLF | minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEAFEDVAVYFSQEEWGLL | |
| | DTAQRALYRRVMLDNFALVASLGLSTSRPRVVIQLERGEE | |
| | PWVPSGTDTTLSRTTYRRRNPGSWSLTEDRDVSGNPKKKR | |
| | KVGIHGVPAADKKYSIGLAIGTNSVGWAVITDEYKVPSKK | |
| | FKVLGNTDRHSIKKNLIGALLFDSGETAEATRLKRTARRR | |
| | YTRRKNRICYLQEIFSNEMAKVDDSFFHRLEESFLVEEDK | |
| | KHERHPIFGNIVDEVAYHEKYPTIYHLRKKLVDSTDKADL | |
| | RLIYLALAHMIKFRGHFLIEGDLNPDNSDVDKLFIQLVQT | |
| | YNQLFEENPINASGVDAKAILSARLSKSRRLENLIAQLPG | |
| | EKKNGLFGNLIALSLGLTPNFKSNFDLAEDAKLQLSKDTY | |
| | DDDLDNLLAQIGDQYADLFLAAKNLSDAILLSDILRVNTE | |
| | ITKAPLSASMIKRYDEHHQDLTLLKALVRQQLPEKYKEIF | |
| | FDQSKNGYAGYIDGGASQEEFYKFIKPILEKMDGTEELLV | |
| | KLNREDLLRKQRTFDNGSIPHQIHLGELHAILRRQEDFYP | |
| | FLKDNREKIEKILTFRIPYYVGPLARGNSRFAWMTRKSEE | |
| | TITPWNFEEVVDKGASAQSFIERMTNFDKNLPNEKVLPKH | |
| | SLLYEYFTVYNELTKVKYVTEGMRKPAFLSGEQKKAIVDL | |
| | LFKTNRKVTVKQLKEDYFKKIECFDSVEISGVEDRFNASL | |
| | GTYHDLLKIIKDKDFLDNEENEDILEDIVLTLTLFEDREM | |
| | IEERLKTYAHLFDDKVMKQLKRRRYTGWGRLSRKLINGIR | |
| | DKQSGKTILDFLKSDGFANRNFMQLIHDDSLTFKEDIQKA | |
| | QVSGQGDSLHEHIANLAGSPAIKKGILQTVKVVDELVKVM | |
| | GRHKPENIVIEMARENQTTQKGQKNSRERMKRIEEGIKEL | |
| | GSQILKEHPVENTQLQNEKLYLYYLQNGRDMYVDQELDIN | |
| | RLSDYDVDAIVPQSFLKDDSIDNKVLTRSDKNRGKSDNVP | |
| | SEEVVKKMKNYWRQLLNAKLITQRKFDNLTKAERGGLSEL | |
| | DKAGFIKRQLVETRQITKHVAQILDSRMNTKYDENDKLIR | |
| | EVKVITLKSKLVSDFRKDFQFYKVREINNYHHAHDAYLNA | |
| | VVGTALIKKYPKLESEFVYGDYKVYDVRKMIAKSEQEIGK | |
| | ATAKYFFYSNIMNFFKTEITLANGEIRKRPLIETNGETGE | |
| | IVWDKGRDFATVRKVLSMPQVNIVKKTEVQTGGFSKESIL | |
| | PKRNSDKLIARKKDWDPKKYGGFDSPTVAYSVLVVAKVEK | |
| | GKSKKLKSVKELLGITIMERSSFEKNPIDFLEAKGYKEVK | |
| | KDLIIKLPKYSLFELENGRKRMLASAGELQKGNELALPSK | |
| | YVNFLYLASHYEKLKGSPEDNEQKQLFVEQHKHYLDEIIE | |
| | QISEFSKRVILADANLDKVLSAYNKHRDKPIREQAENIIH | |
| | LFTLTNLGAPAAFKYFDTTIDRKRYTSTKEVLDATLIHQS | |
| | ITGLYETRIDLSQLGGD | |
| 379 | NHDQEFDPPKVYPPVPAEKRKPIRVLSLFDGIATGLLVLK | D3AL-XTEN80- |
| | DLGIQVDRYIASEVCEDSITVGMVRHQGKIMYVGDVRSVT | EZH2-dSpCas9 - |
| | QKHIQEWGPFDLVIGGSPCNDLSIVNPARKGLYEGTGRLF | minimal |
| | FEFYRLLHDARPKEGDDRPFFWLFENVVAMGVSDKRDISR | |
| | FLESNPVMIDAKEVSAAHRARYFWGNLPGMNRPLASTVND | |
| | KLELQECLEHGRIAKFSKVRTITTRSNSIKQGKDQHFPVF | |
| | MNEKEDILWCTEMERVFGFPVHYTDVSNMSRLARQRLLGR | |
| | SWSVPVIRHLFAPLKEYFACVSSGNSNANSRGPSFSSGLV | |
| | PLSLRGSHMGPMEIYKTVSAWKRQPVRVLSLFRNIDKVLK | |
| | SLGFLESGSGSGGGTLKYVEDVTNVVRRDVEKWGPFDLVY | |
| | GSTQPLGSSCDRCPGWYMFQFHRILQYALPRQESQRPFFW | |
| | IFMDNLLLTEDDQETTTRFLQTEAVTLQDVRGRDYQNAMR | |
| | VWSNIPGLKSKHAPLTPKEEEYLQAQVRSRSKLDAPKVDL | |
| | LVKNCLLPLREYFKYFSQNSLPLGGPSSGAPPPSGGSPAG | |
| | SPTSTEEGTSESATPESGPGTSTEPSEGSAPGSPAGSPTS | |
| | TEEGTSTEPSEGSAPGTSTEPSEGQTGKKSEKGPVCWRKR | |
| | VKSEYMRLRQLKRFRRADEVKTMFSSNRQKILERTETLNQ | |
| | EWKQRRIQPVHIMTSVSSLRGTRECSVTSDLDFPAQVIPL | |
| | KTLNAVASVPIMYSWSPLQQNFMVEDETVLHNIPYMGDEV | |
| | LDQDGTFIEELIKNYDGKVHGDRECGFINDEIFVELVNAL | |
| | GQYNDDDDDDDGDDPDEREEKQKDLEDNRDDKETCPPRKF | |
| | PADKIFEAISSMFPDKGTAEELKEKYKELTEQQLPGALPP | |
| | ECTPNIDGPNAKSVQREQSLHSFHTLFCRRCFKYDCFLHP | |
| | FHATPNTYKRKNTETALDNKPCGPQCYQHLEGAKEFAAAL | |
| | TAERIKTPPKRPGGRRRGRLPNNSSRPSTPTISVLESKDT | |
| | DSDREAGTETGGENNDKEEEEKKDETSSSSEANSRCQTPI | |
| | KMKPNIEPPENVEWSGAEASMFRVLIGTYYDNFCAIARLI | |
| | GTKTCRQVYEFRVKESSIIAPVPTEDVDTPPRKKKRKHRL | |
| | WAAHCRKIQLKKDGSSNHVYNYQPCDHPRQPCDSSCPCVI | |
| | AQNFCEKFCQCSSECQNRFPGCRCKAQCNTKQCPCYLAVR | |
| | ECDPDLCLTCGAADHWDSKNVSCKNCSIQRGSKKHLLLAP | |
| | SDVAGWGIFIKDPVQKNEFISEYCGEIISQDEADRRGKVY | |
| | DKYMCSFLENLNNDFVVDATRKGNKIRFANHSVNPNCYAK | |
| | VMMVNGDHRIGIFAKRAIQTGEELFFDYRYSQADALKYVG | |
| | IEREMEIPSTGGSGGSGGSGGSGGSGRPNPKKKRKVGIHG | |
| | VPAADKKYSIGLAIGTNSVGWAVITDEYKVPSKKFKVLGN | |
| | TDRHSIKKNLIGALLFDSGETAEATRLKRTARRRYTRRKN | |
| | RICYLQEIFSNEMAKVDDSFFHRLEESFLVEEDKKHERHP | |
| | IFGNIVDEVAYHEKYPTIYHLRKKLVDSTDKADLRLIYLA | |
| | LAHMIKFRGHFLIEGDLNPDNSDVDKLFIQLVQTYNQLFE | |
| | ENPINASGVDAKAILSARLSKSRRLENLIAQLPGEKKNGL | |
| | FGNLIALSLGLTPNFKSNFDLAEDAKLQLSKDTYDDDLDN | |
| | LLAQIGDQYADLFLAAKNLSDAILLSDILRVNTEITKAPL | |
| | SASMIKRYDEHHQDLTLLKALVRQQLPEKYKEIFFDQSKN | |
| | GYAGYIDGGASQEEFYKFIKPILEKMDGTEELLVKLNRED | |
| | LLRKQRTFDNGSIPHQIHLGELHAILRRQEDFYPFLKDNR | |
| | EKIEKILTFRIPYYVGPLARGNSRFAWMTRKSEETITPWN | |
| | FEEVVDKGASAQSFIERMTNFDKNLPNEKVLPKHSLLYEY | |
| | FTVYNELTKVKYVTEGMRKPAFLSGEQKKAIVDLLFKTNR | |
| | KVTVKQLKEDYFKKIECFDSVEISGVEDRFNASLGTYHDL | |
| | LKIIKDKDFLDNEENEDILEDIVLTLTLFEDREMIEERLK | |
| | TYAHLFDDKVMKQLKRRRYTGWGRLSRKLINGIRDKQSGK | |
| | TILDFLKSDGFANRNFMQLIHDDSLTFKEDIQKAQVSGQG | |
| | DSLHEHIANLAGSPAIKKGILQTVKVVDELVKVMGRHKPE | |
| | NIVIEMARENQTTQKGQKNSRERMKRIEEGIKELGSQILK | |
| | EHPVENTQLQNEKLYLYYLQNGRDMYVDQELDINRLSDYD | |
| | VDAIVPQSFLKDDSIDNKVLTRSDKNRGKSDNVPSEEVVK | |
| | KMKNYWRQLLNAKLITQRKFDNLTKAERGGLSELDKAGFI | |
| | KRQLVETRQITKHVAQILDSRMNTKYDENDKLIREVKVIT | |
| | LKSKLVSDFRKDFQFYKVREINNYHHAHDAYLNAVVGTAL | |
| | IKKYPKLESEFVYGDYKVYDVRKMIAKSEQEIGKATAKYF | |
| | FYSNIMNFFKTEITLANGEIRKRPLIETNGETGEIVWDKG | |
| | RDFATVRKVLSMPQVNIVKKTEVQTGGFSKESILPKRNSD | |
| | KLIARKKDWDPKKYGGFDSPTVAYSVLVVAKVEKGKSKKL | |
| | KSVKELLGITIMERSSFEKNPIDFLEAKGYKEVKKDLIIK | |
| | LPKYSLFELENGRKRMLASAGELQKGNELALPSKYVNFLY | |
| | LASHYEKLKGSPEDNEQKQLFVEQHKHYLDEIIEQISEFS | |
| | KRVILADANLDKVLSAYNKHRDKPIREQAENIIHLFTLTN | |
| | LGAPAAFKYFDTTIDRKRYTSTKEVLDATLIHQSITGLYE | |
| | TRIDLSQLGGD | |
| 380 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-KOX1(2- |
| | VGTVKHEGQIKYVNDVRKITKKNIEEWGPFDLVIGGSPCN | 99) - minimal |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASDAKSLTAWSRTLVTFKDVFVDFTREEWKLLDTA | |
| | QQILYRNVMLENYKNLVSLGYQLTKPDVILRLEKGEEPWL | |
| | VEREIHQETHPDSETAFEIKSSV | |
| 381 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-KOX1(1- |
| | VGTVKHEGQIKYVNDVRKITKKNIEEWGPFDLVIGGSPCN | 72) - minimal |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASMDAKSLTAWSRTLVTFKDVFVDFTREEWKLLDT | |
| | AQQIVYRNVMLENYKNLVSLGYQLTKPDVILRLEKGEEP | |
| 382 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-ZIM3 - |
| | VGTVKHEGQIKYVNDVRKITKKNIEEWGPFDLVIGGSPCN | minimal |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASNNSQGRVTFEDVTVNFTQGEWQRLNPEQRNLYR | |
| | DVMLENYSNLVSVGQGETTKPDVILRLEQGKEPWLEEEEV | |
| | LGSGRAEKNGDIGGQIWKPKDVKESL | |
| 383 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-ZNF324 - |
| | VGTVKHEGQIKYVNDVRKITKKNIEEWGPFDLVIGGSPCN | minimal |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASAFEDVAVYFSQEEWGLLDTAQRALYRRVMLDNF | |
| | ALVASLGLSTSRPRVVIQLERGEEPWVPSGTDTTLSRTTY | |
| | RRRNPGSWSLTEDRDVSG | |
| 384 | VLRRRKDWNMRLQDFFTTDPDLEEFQEPPKLYPAIPAAKR | D3BL-XTEN80- |
| | RPIRVLSLFDGIATGYLVLKELGIKVEKYIASEVCAESIA | dSpCas9-EZH2 - |
| | VGTVKHEGQIKYVNDVRKITKKNIEEWGPFDLVIGGSPCN | minimal |
| | DLSNVNPARKGLYEGTGRLFFEFYHLLNYTRPKEGDNRPF | |
| | FWMFENVVAMKVNDKKDISRFLACNPVMIDAIKVSAAHRA | |
| | RYFWGNLPGMNRPVMASKNDKLELQDCLEFSRTAKLKKVQ | |
| | TITTKSNSIRQGKNQLFPVVMNGKDDVLWCTELERIFGFP | |
| | AHYTDVSNMGRGARQKLLGRSWSVPVIRHLFAPLKDYFAC | |
| | ESSGNSNANSRGPSFSSGLVPLSLRGSHMGPMEIYKTVSA | |
| | WKRQPVRVLSLFRNIDKVLKSLGFLESGSGSGGGTLKYVE | |
| | DVTNVVRRDVEKWGPFDLVYGSTQPLGSSCDRCPGWYMFQ | |
| | FHRILQYALPRQESQRPFFWIFMDNLLLTEDDQETTTRFL | |
| | QTEAVTLQDVRGRDYQNAMRVWSNIPGLKSKHAPLTPKEE | |
| | EYLQAQVRSRSKLDAPKVDLLVKNCLLPLREYFKYFSQNS | |
| | LPLGGPSSGAPPPSGGSPAGSPTSTEEGTSESATPESGPG | |
| | TSTEPSEGSAPGSPAGSPTSTEEGTSTEPSEGSAPGTSTE | |
| | PSENPKKKRKVGIHGVPAADKKYSIGLAIGTNSVGWAVIT | |
| | DEYKVPSKKFKVLGNTDRHSIKKNLIGALLFDSGETAEAT | |
| | RLKRTARRRYTRRKNRICYLQEIFSNEMAKVDDSFFHRLE | |
| | ESFLVEEDKKHERHPIFGNIVDEVAYHEKYPTIYHLRKKL | |
| | VDSTDKADLRLIYLALAHMIKFRGHFLIEGDLNPDNSDVD | |
| | KLFIQLVQTYNQLFEENPINASGVDAKAILSARLSKSRRL | |
| | ENLIAQLPGEKKNGLFGNLIALSLGLTPNFKSNFDLAEDA | |
| | KLQLSKDTYDDDLDNLLAQIGDQYADLFLAAKNLSDAILL | |
| | SDILRVNTEITKAPLSASMIKRYDEHHQDLTLLKALVRQQ | |
| | LPEKYKEIFFDQSKNGYAGYIDGGASQEEFYKFIKPILEK | |
| | MDGTEELLVKLNREDLLRKQRTFDNGSIPHQIHLGELHAI | |
| | LRRQEDFYPFLKDNREKIEKILTFRIPYYVGPLARGNSRF | |
| | AWMTRKSEETITPWNFEEVVDKGASAQSFIERMTNFDKNL | |
| | PNEKVLPKHSLLYEYFTVYNELTKVKYVTEGMRKPAFLSG | |
| | EQKKAIVDLLFKTNRKVTVKQLKEDYFKKIECFDSVEISG | |
| | VEDRENASLGTYHDLLKIIKDKDFLDNEENEDILEDIVLT | |
| | LTLFEDREMIEERLKTYAHLFDDKVMKQLKRRRYTGWGRL | |
| | SRKLINGIRDKQSGKTILDFLKSDGFANRNFMQLIHDDSL | |
| | TFKEDIQKAQVSGQGDSLHEHIANLAGSPAIKKGILQTVK | |
| | VVDELVKVMGRHKPENIVIEMARENQTTQKGQKNSRERMK | |
| | RIEEGIKELGSQILKEHPVENTQLQNEKLYLYYLQNGRDM | |
| | YVDQELDINRLSDYDVDAIVPQSFLKDDSIDNKVLTRSDK | |
| | NRGKSDNVPSEEVVKKMKNYWRQLLNAKLITQRKFDNLTK | |
| | AERGGLSELDKAGFIKRQLVETRQITKHVAQILDSRMNTK | |
| | YDENDKLIREVKVITLKSKLVSDFRKDFQFYKVREINNYH | |
| | HAHDAYLNAVVGTALIKKYPKLESEFVYGDYKVYDVRKMI | |
| | AKSEQEIGKATAKYFFYSNIMNFFKTEITLANGEIRKRPL | |
| | IETNGETGEIVWDKGRDFATVRKVLSMPQVNIVKKTEVQT | |
| | GGFSKESILPKRNSDKLIARKKDWDPKKYGGFDSPTVAYS | |
| | VLVVAKVEKGKSKKLKSVKELLGITIMERSSFEKNPIDFL | |
| | EAKGYKEVKKDLIIKLPKYSLFELENGRKRMLASAGELQK | |
| | GNELALPSKYVNFLYLASHYEKLKGSPEDNEQKQLFVEQH | |
| | KHYLDEIIEQISEFSKRVILADANLDKVLSAYNKHRDKPI | |
| | REQAENIIHLFTLTNLGAPAAFKYFDTTIDRKRYTSTKEV | |
| | LDATLIHQSITGLYETRIDLSQLGGDSGGKRPAATKKAGQ | |
| | AKKKKASSTGGSGGSGGSGGSGGSGRPGQTGKKSEKGPVC | |
| | WRKRVKSEYMRLRQLKRFRRADEVKTMFSSNRQKILERTE | |
| | TLNQEWKQRRIQPVHIMTSVSSLRGTRECSVTSDLDFPAQ | |
| | VIPLKTLNAVASVPIMYSWSPLQQNFMVEDETVLHNIPYM | |
| | GDEVLDQDGTFIEELIKNYDGKVHGDRECGFINDEIFVEL | |
| | VNALGQYNDDDDDDDGDDPDEREEKQKDLEDNRDDKETCP | |
| | PRKFPADKIFEAISSMFPDKGTAEELKEKYKELTEQQLPG | |
| | ALPPECTPNIDGPNAKSVQREQSLHSFHTLFCRRCFKYDC | |
| | FLHPFHATPNTYKRKNTETALDNKPCGPQCYQHLEGAKEF | |
| | AAALTAERIKTPPKRPGGRRRGRLPNNSSRPSTPTISVLE | |
| | SKDTDSDREAGTETGGENNDKEEEEKKDETSSSSEANSRC | |
| | QTPIKMKPNIEPPENVEWSGAEASMFRVLIGTYYDNFCAI | |
| | ARLIGTKTCRQVYEFRVKESSIIAPVPTEDVDTPPRKKKR | |
| | KHRLWAAHCRKIQLKKDGSSNHVYNYQPCDHPRQPCDSSC | |
| | PCVIAQNFCEKFCQCSSECQNRFPGCRCKAQCNTKQCPCY | |
| | LAVRECDPDLCLTCGAADHWDSKNVSCKNCSIQRGSKKHL | |
| | LLAPSDVAGWGIFIKDPVQKNEFISEYCGEIISQDEADRR | |
| | GKVYDKYMCSFLFNLNNDFVVDATRKGNKIRFANHSVNPN | |
| | CYAKVMMVNGDHRIGIFAKRAIQTGEELFFDYRYSQADAL | |
| | KYVGIEREMEIP | |
| 385 | PCGICTNEVNDDQDAILCEASCQKWFHRICTGMTETAYGL | Bipartite activator - |
| | LTAEASAVWGCDTCMADNGGGGSEGQSDERALLDQLHTLL | PYGO1 NCOA3 |
| | SNTDATGLEEIDRALGIPELVNQGQALEPKQD | (aa) |
| 386 | DIDECASNPCRKGATCINGVNGFRCICPEGPHHPSCYNGG | Bipartite activator - |
| | GGSEGQSDERALLDQLHTLLSNTDATGLEEIDRALGIPEL | NOTCH2 NCOA3 |
| | VNQGQALEPKQD | (aa) |
| 387 | EGQSDERALLDQLHTLLSNTDATGLEEIDRALGIPELVNQ | Bipartite activator - |
| | GQALEPKQDGGGGSEGQSDERALLDQLHTLLSNTDATGLE | NCOA3 NCOA3 |
| | EIDRALGIPELVNQGQALEPKQD | (aa) |
| 388 | WFHGAISREDAENLLESQPLGSFLIRVSHSHVGYTLSYKA | Bipartite activator - |
| | QSSCCHFMVKLLDDGTFMIPGEKVAHTSLDALVTFHNGGG | HSH2D NCOA3 |
| | GSEGQSDERALLDQLHTLLSNTDATGLEEIDRALGIPELV | (aa) |
| | NQGQALEPKQD | |
| 389 | HEKFPSDLDLDMFNGSLECDMESIIRSELMDADGLDFNFD | Bipartite activator - |
| | SLGGGGSEGQSDERALLDQLHTLLSNTDATGLEEIDRALG | FOXO3 NCOA3 |
| | IPELVNQGQALEPKQD | (aa) |
| 390 | ESPSDEGALLDQLYLALRNFDGLEEIDRALGIPELVSQSQ | Bipartite activator - |
| | AVDPEQFNGGGGSEGQSDERALLDQLHTLLSNTDATGLEE | NCOA2 NCOA3 |
| | IDRALGIPELVNQGQALEPKQD | (aa) |
| 391 | AYTDELVELHRRLMALRERNVLQQIVNLIEETGHFNVTNT | Bipartite activator - |
| | TFDFDLFSLDETTVRKLQSCLENGGGGSEGQSDERALLDQ | ENL NCOA3 (aa) |
| | LHTLLSNTDATGLEEIDRALGIPELVNQGQALEPKQD | |
| 392 | PCGICTNEVNDDQDAILCEASCQKWFHRICTGMTETAYGL | Bipartite activator - |
| | LTAEASAVWGCDTCMADNGGGGSHEKFPSDLDLDMFNGSL | PYGO1 FOXO3 |
| | ECDMESIIRSELMDADGLDFNFDSL | (aa) |
| 393 | DIDECASNPCRKGATCINGVNGFRCICPEGPHHPSCYNGG | Bipartite activator - |
| | GGSHEKFPSDLDLDMFNGSLECDMESIIRSELMDADGLDF | NOTCH2 FOXO3 |
| | NFDSL | (aa) |
| 394 | EGQSDERALLDQLHTLLSNTDATGLEEIDRALGIPELVNQ | Bipartite activator - |
| | GQALEPKQDNGGGGSHEKFPSDLDLDMFNGSLECDMESII | NCOA3 FOXO3 |
| | RSELMDADGLDFNFDSL | (aa) |
| 395 | WFHGAISREDAENLLESQPLGSFLIRVSHSHVGYTLSYKA | Bipartite activator - |
| | QSSCCHFMVKLLDDGTFMIPGEKVAHTSLDALVTFHNGGG | HSH2D FOXO3 |
| | GSHEKFPSDLDLDMFNGSLECDMESIIRSELMDADGLDFN | (aa) |
| | FDSL | |
| 396 | HEKFPSDLDLDMFNGSLECDMESIIRSELMDADGLDFNFD | Bipartite activator - |
| | SLNGGGGSHEKFPSDLDLDMFNGSLECDMESIIRSELMDA | FOXO3 FOXO3 |
| | DGLDFNFDSL | (aa) |
| 397 | ESPSDEGALLDQLYLALRNFDGLEEIDRALGIPELVSQSQ | Bipartite activator - |
| | AVDPEQFNGGGGSHEKFPSDLDLDMFNGSLECDMESIIRS | NCOA2 FOXO3 |
| | ELMDADGLDFNFDSL | (aa) |
| 398 | AYTDELVELHRRLMALRERNVLQQIVNLIEETGHFNVTNT | Bipartite activator - |
| | TFDFDLFSLDETTVRKLQSCLENGGGGSHEKFPSDLDLDM | ENL FOXO3 (aa) |
| | FNGSLECDMESIIRSELMDADGLDFNFDSL | |
| 399 | PCGICTNEVNDDQDAILCEASCQKWFHRICTGMTETAYGL | Tripartite activator |
| | LTAEASAVWGCDTCMADNGGGGSHEKFPSDLDLDMFNGSL | - PYGO1 FOXO3 |
| | ECDMESIIRSELMDADGLDFNFDSLGGGGSEGQSDERALL | NCOA3 (aa) |
| | DQLHTLLSNTDATGLEEIDRALGIPELVNQGQALEPKQD | |
| 400 | DIDECASNPCRKGATCINGVNGFRCICPEGPHHPSCYNGG | Tripartite activator |
| | GGSHEKFPSDLDLDMFNGSLECDMESIIRSELMDADGLDF | - NOTCH2 |
| | NFDSLGGGGSEGQSDERALLDQLHTLLSNTDATGLEEIDR | FOXO3 NCOA3 |
| | ALGIPELVNQGQALEPKQD | (aa) |
| 401 | EGQSDERALLDQLHTLLSNTDATGLEEIDRALGIPELVNQ | Tripartite activator |
| | GQALEPKQDNGGGGSHEKFPSDLDLDMFNGSLECDMESII | - NCOA3 FOXO3 |
| | RSELMDADGLDFNFDSLGGGGSEGQSDERALLDQLHTLLS | NCOA3 (aa) |
| | NTDATGLEEIDRALGIPELVNQGQALEPKQD | |
| 402 | WFHGAISREDAENLLESQPLGSFLIRVSHSHVGYTLSYKA | Tripartite activator |
| | QSSCCHFMVKLLDDGTFMIPGEKVAHTSLDALVTFHNGGG | - HSH2D FOXO3 |
| | GSHEKFPSDLDLDMFNGSLECDMESIIRSELMDADGLDFN | NCOA3 (aa) |
| | FDSLGGGGSEGQSDERALLDQLHTLLSNTDATGLEEIDRA | |
| | LGIPELVNQGQALEPKQD | |
| 403 | HEKFPSDLDLDMFNGSLECDMESIIRSELMDADGLDFNFD | Tripartite activator |
| | SLNGGGGSHEKFPSDLDLDMFNGSLECDMESIIRSELMDA | - FOX03 FOXO3 |
| | DGLDFNFDSLGGGGSEGQSDERALLDQLHTLLSNTDATGL | NCOA3 (aa) |
| | EEIDRALGIPELVNQGQALEPKQD | |
| 404 | ESPSDEGALLDQLYLALRNFDGLEEIDRALGIPELVSQSQ | Tripartite activator |
| | AVDPEQFNGGGGSHEKFPSDLDLDMFNGSLECDMESIIRS | - NCOA2 FOXO3 |
| | ELMDADGLDFNFDSLGGGGSEGQSDERALLDQLHTLLSNT | NCOA3 (aa) |
| | DATGLEEIDRALGIPELVNQGQALEPKQD | |
| 405 | AYTDELVELHRRLMALRERNVLQQIVNLIEETGHFNVTNT | Tripartite activator |
| | TFDFDLFSLDETTVRKLQSCLENGGGGSHEKFPSDLDLDM | - ENL FOXO3 |
| | FNGSLECDMESIIRSELMDADGLDFNFDSLGGGGSEGQSD | NCOA3 (aa) |
| | ERALLDQLHTLLSNTDATGLEEIDRALGIPELVNQGQALE | |
| | PKQD | |
| 406 | EGQSDERALLDQLHTLLSNTDATGLEEIDRALGIPELVNQ | Tripartite activator |
| | GQALEPKQDGGGGSHEKFPSDLDLDMFNGSLECDMESIIR | - NCOA3, |
| | SELMDADGLDFNFDSLNGGGGSHEKFPSDLDLDMFNGSLE | FOXO3, |
| | CDMESIIRSELMDADGLDFNFDSL | FOXO3 (aa) |
| 407 | KSKKGRQEALERLKKAKAGEKYKYEVEDFTGVYEEVDEEQ | DPOLA (aa) |
| | YSKLVQARQDDDWIVDDDGIGYVEDGREIFDDDLEDDALD | |
| 408 | SKPEKILKKGTYDKAYTDELVELHRRLMALRERNVLQQIV | ENL (aa) |
| | NLIEETGHFNVTNTTFDFDLFSLDETTVRKLQSCLEAVAT | |
| 409 | DSLSGSSLYSTSANLPVMGHEKFPSDLDLDMFNGSLECDM | FOXO3 (aa) |
| | ESIIRSELMDADGLDFNFDSLISTQNVVGLNVGNFTGAKQ | |
| 410 | PEWFHGAISREDAENLLESQPLGSFLIRVSHSHVGYTLSY | HSH2D (aa) |
| | KAQSSCCHFMVKLLDDGTFMIPGEKVAHTSLDALVTFHQQ | |
| 411 | PFGSSPDDLLCPHPAAESPSDEGALLDQLYLALRNFDGLE | NCOA2 (aa) |
| | EIDRALGIPELVSQSQAVDPEQFSSQDSNIMLEQKAPVFP | |
| 412 | LRNSLDDLVGPPSNLEGQSDERALLDQLHTLLSNTDATGL | NCOA3 (aa) |
| | EEIDRALGIPELVNQGQALEPKQDAFQGQEAAVMMDQKAG | |
| 413 | FRNQYDNDVTVWSPQGRIHQIEYAMEAVKQGSATVGLKSK | PSA1 (aa) |
| | THAVLVALKRAQSELAAHQKKILHVDNHIGISIAGLTADA | |
| 414 | RHGHSSSDPVYPCGICTNEVNDDQDAILCEASCQKWFHRI | PYGO1 (aa) |
| | CTGMTETAYGLLTAEASAVWGCDTCMADKDVQLMRTRETF | |
| 415 | GPMRLYVGSLHFNITEDMLRGIFEPFGRIESIQLMMDSET | RBM39 (aa) |
| | GRSKGYGFITFSDSECAKKALEQLNGFELAGRPMKVGHVT | |
| 416 | TLIRKADLENHNKDGGFWTVIDGKVYDIKDFQTQSLTGNS | HERC2 (aa) |
| | ILAQFAGEDPVVALEAALQFEDTRESMHAFCVGQYLEPDQ | |
| 417 | AEEFVTLKDVGMDFTLGDWEQLGLEQGDTFWDTALDNCQD | ZNF473 (AA) |
| | LFLLDPPRPNLTSHPDGSEDLEPLAGGSPEATSPDVTETK | |
| 418 | ECSEAGLLQEGVQPEEFVAIADYAATDETQLSFLRGEKIL | ANM2 (AA) |
| | ILRQTTADWWWGERAGCCGYIPANHVGKHVDEYDPEDTWQ | |
| 419 | PRPELPLPEGWEEARDFDGKVYYIDHTNRTTSWIDPRDRY | KIBRA (AA) |
| | TKPLTFADCISDELPLGWEEAYDPQVGDYFIDHNTKTTQI | |
| 420 | LVGSSLEGAVTPQTSAWLPPTSAEHDHSLSCVVTPQDGET | IKKA (AA) |
| | SAQMIEENLNCLGHLSTIIHEANEEQGNSMMNLDWSWLTE | |
| 421 | GSPSYGSPEDTDSFWNPNAFETDSDLPAGWMRVQDTSGTY | APBB1 (AA) |
| | YWHIPTGTTQWEPPGRASPSQGSSPQEESQLTWTGFAHGE | |
| 422 | GNREEQNLSDLLSPICEVANNIEQNAQENENESQVSTDES | SMN2 (AA) |
| | ENSRSPGNKSDNIKPKSAPWNSFLPPPPPMPGPRLGPGKP | |
| 423 | LMDSLPGNFEITTSTGFLTDLTLDDILFADIDTSMYDFDP | SERTAD2 (AA) |
| | CTSSSGTASKMAPVSADDLLKTLAPYSSQPVTPSQPFKMD | |
| 424 | FYIPVQIPGYQYVSPEGNCIEHVQPTSAFIQQPFIDEDPD | MYBA (AA) |
| | KEKKIKELEMLLMSAENEVRRKRIPSQPGSFSSWSGSFLM | |

What is claimed:

1. An epigenetic-modifying DNA-targeting system comprising at least one DNA-targeting module for repressing transcription of one or more genes in a T cell, wherein each of the at least one DNA-targeting module comprises a fusion protein comprising:
(a) a DNA-binding domain capable of being targeted to a target site for one of the one or more genes, wherein the one or more genes are selected from the group consisting of CBLB, CCNC, CD5, CISH, DGKZ, ELOB, FAS, Fli1, GATA3, KDM1A, MED12, MYB, PRDM1, TGFBR2, and RASA2; and
(b) at least one transcriptional repressor effector domain for repressing transcription of the one or more genes in a T cell.

2. An epigenetic-modifying DNA-targeting system comprising at least one DNA-targeting module for increasing transcription of one or more genes in a T cell, wherein each of the at least one DNA-targeting module comprises a fusion protein comprising:
(a) a DNA-binding domain capable of being targeted to a target site for one of the one or more genes, wherein the one or more genes are selected from the group consisting of BATF, CD28, EOMES, IL-2, IL2RB, IRF4, LAT, LCP2, TBX21, and VAV1; and
(b) at least one transcriptional activator effector domain for increasing transcription of the one or more genes in a T cell.

3. The epigenetic-modifying DNA-targeting system of claim 1 or claim 2, wherein transient delivery of the epigenetic-modifying DNA-targeting system to the T cell promotes increased T cell effector function upon T cell stimulation, optionally increased compared to a T cell that has not been delivered the epigenetic-modifying DNA-targeting system.

4. The epigenetic-modifying DNA-targeting system of any of claims 1-3, wherein the T cell effector function is characterized by an activity selected from the group consisting of IL-2 production, IFN-gamma production, TNF-alpha production, T cell proliferation or a combination of any of the foregoing.

5. The epigenetic-modifying DNA-targeting system of any of claims 1-4, wherein the T cell effector function is characterized by IL-2 production.

6. The epigenetic-modifying DNA-targeting system of any of claims 1-4, wherein the T cell effector function is characterized by IFN-gamma production.

7. The epigenetic-modifying DNA-targeting system of any of claims 1-4, wherein the T cell effector function is characterized by IL-2 production and IFN-gamma production.

8. The epigenetic-modifying DNA-targeting system of any of claims 1-4, wherein the T cell effector function is characterized by polyfunctional production of IL-2, IFN-gamma and TNF-alpha.

9. The epigenetic-modifying DNA-targeting system of any of claims 4-8, wherein the T cell effector function is characterized by activity that further comprises T cell proliferation.

10. The epigenetic modifying DNA-targeting system of any of claims 4-9, wherein the T cell effector function is characterized by activity that further comprises killing of target cells.

11. The epigenetic-modifying DNA-targeting system of any of claims 4-10, wherein the T cell effector function is characterized by activity that further comprises T cell persistence.

12. The epigenetic-modifying DNA-targeting system of any of claims 3-11, wherein the increased T cell effector function occurs 48 hours or more after the transient delivery of the epigenetic-modifying DNA-targeting system to the T cell.

13. The epigenetic-modifying DNA-targeting system of any of claims 3-12, wherein the increased T cell effector function occurs up to 6 days, up to 9 days, up to 12 days, up to 15 days, up to 21 days, up to 28 days, up to 35 days, up to 42 days, up to 49 days, up to 56 days, up to 63 days, up to 71 days or more after the transient delivery of the epigenetic-modifying DNA-targeting system to the T cell.

14. The epigenetic-modifying DNA-targeting system of any of claims 3-13, wherein the T cell stimulation is with an anti-CD3 and anti-CD28 activation reagent.

15. The epigenetic-modifying DNA-targeting system of any of claims 3-14, wherein the T cell expresses an engineered antigen receptor, optionally a chimeric antigen receptor or a T cell receptor (eTCR).

16. The epigenetic-modifying DNA-targeting system of claim 15, wherein the engineered antigen receptor is a chimeric antigen receptor (CAR) or engineered T cell receptor (eTCR) directed against an antigen and the T cell stimulation is an antigen-specific stimulation of the CAR or eTCR, optionally wherein the T cell stimulation is with antigen-expressing target cells.

17. The epigenetic-modifying DNA-targeting system of any of claims 3-16, wherein the T cell expresses a chimeric antigen receptor (CAR) directed against an antigen and the T cell stimulation is an antigen-specific stimulation of the CAR, optionally wherein the T cell stimulation is with antigen-expressing target cells.

18. The epigenetic-modifying DNA-targeting system of any of claims 3-17, wherein the T cell stimulation is a restimulation after at least one prior T cell stimulation of the T cells.

19. The epigenetic-modifying DNA-targeting system of any of claims 1-18, wherein the DNA-targeting system does not introduce a genetic disruption or a DNA break.

20. The epigenetic-modifying DNA-targeting system of any of claims 1-19, wherein the fusion protein of each DNA-targeting module comprises a DNA-binding domain selected from: a Clustered Regularly Interspaced Short Palindromic Repeats associated (Cas) protein or a variant thereof; a zinc finger protein (ZFP); a transcription activator-like effector (TALE); a meganuclease; a homing endonuclease; or an I-SceI enzyme or a variant thereof, optionally wherein the DNA-binding domain comprises a catalytically inactive variant of any of the foregoing.

21. The epigenetic-modifying DNA-targeting system of any of claims 1-20, wherein the at least one DNA-targeting module is a single DNA-targeting module that targets a target site for one of the one or more genes.

22. The epigenetic-modifying DNA-targeting system of any of claims 1-20, wherein:
the at least one DNA targeting module is a plurality of DNA-targeting modules for repressing transcription of one or more genes in a T cell, wherein each DNA-targeting module targets a target site for one of the one or more genes; or
the at least one DNA targeting module is a plurality of DNA-targeting modules for increasing transcription of one or more genes in a T cell, wherein each DNA-targeting module targets a target site for one of the one or more genes.

23. The epigenetic-modifying DNA-targeting system of claim 22, wherein the plurality of DNA-targeting modules is 2, 3, 4, 5 or 6 DNA-targeting modules.

24. The epigenetic-modifying DNA-targeting system of claim 22 or 23, wherein the plurality of DNA-targeting modules is 2 DNA-targeting modules.

25. The epigenetic-modifying DNA-targeting system of any of claims 22-24, wherein the plurality of DNA-targeting modules for repressing transcription of one or more genes in a T cell target at least a first gene and a second gene, wherein the first and second gene are independently selected from the group consisting of CBLB, CCNC, CD5, CISH, DGKZ, ELOB, FAS, Fli1, GATA3, KDM1A, MED12, MYB, PRDM1, TGFBR2, and RASA2.

26. The epigenetic-modifying DNA-targeting system of any of claims 22-25, wherein the plurality of DNA-targeting modules for repressing transcription of one or more genes in a T cell target at least a first gene and a second gene, wherein the first and second gene are independently selected from the group consisting of CBLB, CISH, MED12, MYB, PRDM1, and RASA2.

27. The epigenetic-modifying DNA-targeting system of claim 22 or 23, wherein the plurality of DNA-targeting modules for repressing transcription of one or more genes in a T cell target at least a first gene, a second gene, and a third gene, wherein the first, second and third gene are independently selected from the group consisting of CBLB, CCNC, CD5, CISH, DGKZ, ELOB, FAS, Fli1, GATA3, KDM1A, MED12, MYB, PRDM1, TGFBR2, and RASA2.

28. The epigenetic-modifying DNA-targeting system of claim 22-23 or 27, wherein the plurality of DNA-targeting modules for repressing transcription of one or more genes in a T cell target at least a first gene, a second gene, and a third gene, wherein the first, second and third gene are independently selected from the group consisting of CBLB, CISH, MED12, MYB, PRDM1, and RASA2.

29. The epigenetic-modifying DNA-targeting system of any of claims 22-24, wherein the plurality of DNA-targeting modules for increasing transcription of one or more genes in a T cell target at least a first gene and a second gene, wherein the first and second gene are independently selected from the group consisting of BATF, CD28, EOMES, IL-2, IL2RB, IRF4, LAT, LCP2, TBX21, and VAV1.

30. The epigenetic-modifying DNA-targeting system of any of claims 22-24 or 29, wherein the plurality of DNA-targeting modules for increasing transcription of one or more genes in a T cell target at least a first gene and a second gene, wherein the first and second gene are independently selected from the group consisting of EOMES, IL-2, LCP2, and TBX21.

31. The epigenetic-modifying DNA-targeting system of any of claims 22, 23, and 29, wherein the plurality of DNA-targeting modules for increasing transcription of one or more genes in a T cell target at least a first gene, a second gene, and a third gene, wherein the first, second and third gene are independently selected from the group consisting of BATF, CD28, EOMES, IL-2, IL2RB, IRF4, LAT, LCP2, TBX21, and VAV1.

32. The epigenetic-modifying DNA-targeting system of any of claims 22, 23, 29, or 31, wherein the plurality of DNA-targeting modules for increasing transcription of one or more genes in a T cell target at least a first gene, a second gene, and a third gene, wherein the first, second and third gene are independently selected from the group consisting of EOMES, IL-2, LCP2, and TBX21.

33. The epigenetic-modifying DNA-targeting system of claim 25 or 27, wherein the plurality of DNA-targeting modules target a combination of genes selected from: CBLB and CCNC; CBLB and CD5; CBLB and CISH; CBLB and DGKZ; CBLB and ELOB; CBLB and FAS; CBLB and Fli1; CBLB and GATA3; CBLB and KDM1A; CBLB and MED12; CBLB and MYB; CBLB and PRDM1; CBLB and RASA2; CD5 and CISH; CD5 and MYB; CISH and DGKZ; CISH and MYB; CISH and RASA2; GATA3 and CD5; GATA3 and CISH; GATA3 and MYB; MED12 and CBLB; MED12 and CD5; MED12 and CISH; MED12 and DGKZ; MED12 and ELOB; MED12 and GATA3; MED12 and MYB; MED12 and PRDM1; MED12 and RASA2; MYB and RASA2; PRDM1 and CISH; PRDM1 and GATA3; PRDM1 and MYB; PRDM1 and RASA2; CD5, CISH, and MYB; GATA3, CBLB, and MYB; GATA3, CD5, and MYB; PRDM1, GATA3, and CISH, TGFBR2 and MED12; and TGFBR2, MED12, and CISH.

34. The epigenetic-modifying DNA-targeting system of claim 25-27, or 33, wherein the plurality of DNA-targeting modules target a combination of genes selected from: MED12 and CBLB; MED12 and CISH; CBLB and MYB; and CBLB and RASA2.

35. The epigenetic modifying DNA-targeting system of claim 25 or 27, wherein the first and second gene are CBLB and MYB.

36. The epigenetic modifying DNA-targeting system of claim 25 or 27, wherein the first and second gene are CBLB and MED12.

37. The epigenetic modifying DNA-targeting system of claim 25 or 27, wherein the first and second gene are CBLB and CCNC.

38. The epigenetic-modifying DNA-targeting system of claim 29 or 31, wherein the plurality of DNA-targeting modules target a combination of genes selected from: BATF and IL-2; BATF and VAV1; CD28 and BATF; CD28 and EOMES; CD28 and IL-2; CD28 and LCP2; CD28 and TBX21; CD28 and VAV1; EOMES and BATF; EOMES and LCP2; EOMES and TBX21; EOMES and VAV1; EOMES and IL-2; LCP2 and BATF; LCP2 and IL-2; LCP2 and TBX21; LCP2 and VAV1; TBX21 and BATF; TBX21 and IL-2; TBX21 and TBX21; TBX21 and VAV1; and VAV1 and IL-2.

39. The epigenetic-modifying DNA-targeting system of claim 29 or 31, wherein the first and second gene are IL2RB and VAV1.

40. The epigenetic-modifying DNA-targeting system of 29 or 31, wherein the first and second gene are IL2 and VAV1.

41. The epigenetic-modifying DNA-targeting system of 29 or 31, wherein the first and second gene are IL2 and LCP2.

42. The epigenetic-modifying DNA-targeting system of 29 or 31, wherein the first and second gene are IL2 and TBX21.

43. The epigenetic-modifying DNA-targeting system of 29 or 31, wherein the first and second gene are IL2 and EOMES.

44. The epigenetic-modifying DNA-targeting system of any of claims 1-43, wherein the target site for the gene or for each of the one or more genes is in the gene and/or a regulatory DNA element thereof.

45. The epigenetic-modifying DNA-targeting system of claim 44, wherein the regulatory DNA element is an enhancer or a promoter of the gene.

46. The epigenetic-modifying DNA-targeting system of any of claims 1-45, wherein the target site is within 1000 base pairs (bp) of a transcriptional start site of the gene.

47. The epigenetic-modifying DNA-targeting system of any of claims 1-46, wherein the target site is within 500 base pairs (bp) of a transcriptional start site of the gene.

48. The epigenetic-modifying DNA-targeting system of any of claims 1, 3-27, 33-37, and 44-47, wherein the target site is selected from:
(a) a target site for CD5 having the sequence set forth in any one of SEQ ID NOS:1-3, a contiguous portion thereof of at least 14 nucleotides (nt), or a complementary sequence of any of the foregoing;
(b) a target site for KDM1A having the sequence set forth in any one of SEQ ID NOS:4-6, a contiguous portion thereof of at least 14 nucleotides (nt), or a complementary sequence of any of the foregoing;
(c) a target site for CBLB having the sequence set forth in any one of SEQ ID NOS:10-12, a contiguous portion thereof of at least 14 nucleotides (nt), or a complementary sequence of any of the foregoing;
(d) a target site for DGKZ having the sequence set forth in any one of SEQ ID NOS:13-15, a contiguous portion thereof of at least 14 nucleotides (nt), or a complementary sequence of any of the foregoing;
(e) a target site for MYB having the sequence set forth in any one of SEQ ID NOS:16-18, a contiguous portion thereof of at least 14 nucleotides (nt), or a complementary sequence of any of the foregoing;
(f) a target site for RASA2 having the sequence set forth in any one of SEQ ID NOS:19-21, a contiguous portion thereof of at least 14 nucleotides (nt), or a complementary sequence of any of the foregoing;
(g) a target site for ELOB having the sequence set forth in any one of SEQ ID NOS:22-24, a contiguous portion thereof of at least 14 nucleotides (nt), or a complementary sequence of any of the foregoing;
(h) a target site for GATA3 having the sequence set forth in any one of SEQ ID NOS:25-27, a contiguous portion thereof of at least 14 nucleotides (nt), or a complementary sequence of any of the foregoing;
(i) a target site for CISH having the sequence set forth in any one of SEQ ID NOS:28-30, a contiguous portion thereof of at least 14 nucleotides (nt), or a complementary sequence of any of the foregoing;
(j) a target site for PRDM1 having the sequence set forth in any one of SEQ ID NOS:31-33, a contiguous portion thereof of at least 14 nucleotides (nt), or a complementary sequence of any of the foregoing;
(k) a target site for MED12 having the sequence set forth in any one of SEQ ID NOS:80-90, a contiguous portion thereof of at least 14 nucleotides (nt), or a complementary sequence of any of the foregoing;
(l) a target site for CCNC having the sequence set forth in any one of SEQ ID NOS:102-112, a contiguous portion thereof of at least 14 nucleotides (nt), or a complementary sequence of any of the foregoing;
(m) a target site for FAS having the sequence set forth in any one of SEQ ID NOS:200-205 and 292-295, a contiguous portion thereof of at least 14 nucleotides (nt), or a complementary sequence of any of the foregoing;
(n) a target site for Fli1 having the sequence set forth in any one of SEQ ID NOS:206-211, a contiguous portion thereof of at least 14 nucleotides (nt), or a complementary sequence of any of the foregoing; and
(o) a target site for TGFBR2 having the sequence set forth in any one of SEQ ID NOS:300-302 and 306-308, a contiguous portion thereof of at least 14 nucleotides (nt), or a complementary sequence of any of the foregoing.

49. The epigenetic-modifying DNA-targeting system of any of claims 1, 3-27, 33-37, and 44-47, wherein the target site is selected from:
(a) a target site for CD5 having the sequence set forth in any one of SEQ ID NOS:1-3 or a complementary sequence thereof;
(b) a target site for KDM1A having the sequence set forth in any one of SEQ ID NOS:4-6 or a complementary sequence thereof;
(c) a target site for CBLB having the sequence set forth in any one of SEQ ID NOS:10-12 or a complementary sequence thereof;
(d) a target site for DGKZ having the sequence set forth in any one of SEQ ID NOS:13-15 or a complementary sequence of any of the foregoing;
(e) a target site for MYB having the sequence set forth in any one of SEQ ID NOS:16-18 or a complementary sequence of any of the foregoing;
(f) a target site for RASA2 having the sequence set forth in any one of SEQ ID NOS:19-21 or a complementary sequence of any of the foregoing;
(g) a target site for ELOB having the sequence set forth in any one of SEQ ID NOS:22-24 or a complementary sequence of any of the foregoing;
(h) a target site for GATA3 having the sequence set forth in any one of SEQ ID NOS:25-27 or a complementary sequence of any of the foregoing;
(i) a target site for CISH having the sequence set forth in any one of SEQ ID NOS:28-30 or a complementary sequence of any of the foregoing;
(j) a target site for PRDM1 having the sequence set forth in any one of SEQ ID NOS:31-33 or a complementary sequence of any of the foregoing;
(k) a target site for MED12 having the sequence set forth in any one of SEQ ID NOS:80-90 or a complementary sequence of any of the foregoing;
(l) a target site for CCNC having the sequence set forth in any one of SEQ ID NOS:102-112 or a complementary sequence of any of the foregoing;
(m) a target site for FAS having the sequence set forth in any one of SEQ ID NOS:200-205 and 292-295 or a complementary sequence of any of the foregoing;
(n) a target site for Fli1 having the sequence set forth in any one of SEQ ID NOS:206-211 or a complementary sequence of any of the foregoing; and
(o) a target site for TGFBR2 having the sequence set forth in any one of SEQ ID NOS:300-302 and 306-308 or a complementary sequence of any of the foregoing.

50. The epigenetic-modifying DNA-targeting system of any of claims 1, 3-27, 33-37, and 44-47,
(a) wherein the target site is selected from a target site for CBLB having the sequence set forth in SEQ ID NO:11 or a complementary sequence thereof;
(b) wherein the target site is selected from a target site for MYB having the sequence set forth in SEQ ID NO:18 or a complementary sequence thereof;
(c) wherein the target site is selected from a target site for RASA2 having the sequence set forth in SEQ ID NO:19 or a complementary sequence thereof;
(d) wherein the target site is selected from a target site for CISH having the sequence set forth in SEQ ID NO:28 or a complementary sequence thereof;
(e) wherein the target site is selected from a target site for PRDM1 having the sequence set forth in SEQ ID NO:33 or a complementary sequence thereof; and
(f) wherein the target site is selected from a target site for MED12 having the sequence set forth in SEQ ID NO:81 or a complementary sequence thereof.

51. The epigenetic-modifying DNA-targeting system of any of claims 2-24, 29, 31, and 38-47, wherein the target site is selected from:
(a) a target site for VAV1 having the sequence set forth in any one of SEQ ID NOS:7-9, 156, and 170, a contiguous portion thereof of at least 14 nucleotides (nt), or a complementary sequence of any of the foregoing;
(b) a target site for IL2 having the sequence set forth in SEQ ID NO:78, a contiguous portion thereof of at least 14 nucleotides (nt), or a complementary sequence of any of the foregoing;
(c) a target site for BATF having the sequence set forth in any one of SEQ ID NOS:172-174, a contiguous portion thereof of at least 14 nucleotides (nt), or a complementary sequence of any of the foregoing;
(d) a target site for CD28 having the sequence set forth in any one of SEQ ID NOS:144-146 and 189-191, a contiguous portion thereof of at least 14 nucleotides (nt), or a complementary sequence of any of the foregoing;
(e) a target site for EOMES having the sequence set forth in any one of SEQ ID NOS:147-149, a contiguous portion thereof of at least 14 nucleotides (nt), or a complementary sequence of any of the foregoing;
(f) a target site for IRF4 having the sequence set forth in any one of SEQ ID NOS:175-177, a contiguous portion thereof of at least 14 nucleotides (nt), or a complementary sequence of any of the foregoing;
(g) a target site for LAT having the sequence set forth in any one of SEQ ID NOS:184-186, a contiguous portion thereof of at least 14 nucleotides (nt), or a complementary sequence of any of the foregoing;
(h) a target site for LCP2 having the sequence set forth in any one of SEQ ID NOS:150-152 and 187-188, a contiguous portion thereof of at least 14 nucleotides (nt), or a complementary sequence of any of the foregoing; and
(i) a target site for TBX21 having the sequence set forth in any one of SEQ ID NOS:153-155, a contiguous portion thereof of at least 14 nucleotides (nt), or a complementary sequence of any of the foregoing.

52. The epigenetic-modifying DNA-targeting system of any of claims 2-24, 29, 31, 38-47, and 51, wherein the target site is selected from:
(a) a target site for VAV1 having the sequence set forth in any one of SEQ ID NOS:7-9, 156, and 170, or a complementary sequence of any of the foregoing;
(b) a target site for IL2 having the sequence set forth in SEQ ID NO:78 or a complementary sequence of any of the foregoing;
(c) a target site for BATF having the sequence set forth in any one of SEQ ID NOS:172-174, or a complementary sequence of any of the foregoing;
(d) a target site for CD28 having the sequence set forth in any one of SEQ ID NOS:144-146 and 189-191, or a complementary sequence of any of the foregoing;
(e) a target site for EOMES having the sequence set forth in any one of SEQ ID NOS:147-149, or a complementary sequence of any of the foregoing;
(f) a target site for IRF4 having the sequence set forth in any one of SEQ ID NOS:175-177, or a complementary sequence of any of the foregoing;
(g) a target site for LAT having the sequence set forth in any one of SEQ ID NOS:184-186, or a complementary sequence of any of the foregoing;
(h) a target site for LCP2 having the sequence set forth in any one of SEQ ID NOS:150-152 and 187-188, or a complementary sequence of any of the foregoing; and
(i) a target site for TBX21 having the sequence set forth in any one of SEQ ID NOS:153-155, or a complementary sequence of any of the foregoing.

53. The epigenetic-modifying DNA-targeting system of any of claims 2-24, 29, 31, 38-47, and 52, wherein the target site is selected from:
(a) a target site for IL-2 having the sequence set forth in SEQ ID NO:78, or a complementary sequence of any of the foregoing;
(b) a target site for EOMES having the sequence set forth in SEQ ID NO:149, or a complementary sequence of any of the foregoing;
(c) a target site for LCP2 having the sequence set forth in SEQ ID NO:151, or a complementary sequence of any of the foregoing; and
(d) a target site for TBX21 having the sequence set forth in SEQ ID NO:155, or a complementary sequence of any of the foregoing.

54. The epigenetic-modifying DNA-targeting system of any of claims 1-53, wherein the DNA-binding domain of each of the at least one DNA-targeting module is a Clustered Regularly Interspaced Short Palindromic Repeats associated (Cas) protein or variant thereof, and each of the at least one DNA-targeting module further comprises at least one gRNA for targeting the DNA-binding domain to the target site of the one or more genes.

55. The epigenetic-modifying DNA-targeting system of any of claims 20-54, wherein the Cas protein or variant thereof is a deactivated (dCas) protein.

56. The epigenetic-modifying DNA-targeting system of claim 55, wherein the dCas protein lacks nuclease activity.

57. The epigenetic-modifying DNA-targeting system of claim 55 or 56, wherein the dCas protein is a dCas9 protein.

58. The epigenetic-modifying DNA-targeting system of claim 55 or 56, wherein the dCas protein is a dCas12 protein.

59. The epigenetic-modifying DNA-targeting system of 57, wherein the dCas9 protein is a Staphylococcus aureus dCas9 (dSaCas9) protein.

60. The epigenetic-modifying DNA-targeting system of claim 59, wherein the dSaCas9 comprises at least one amino acid mutation selected from D10A and N580A, with reference to numbering of positions of SEQ ID NO:124.

61. The epigenetic-modifying DNA-targeting system of claim 59 or 60, wherein the dSaCas9 protein comprises the sequence set forth in SEQ ID NO:125, or an amino acid sequence that has at least 90%, 91%, 92%, 93%, 94%, 95%, 96%, 97%, 98%, or 99% sequence identity thereto.

62. The epigenetic-modifying DNA-targeting system of any of claims 59-61, wherein the dSaCas9 is set forth in SEQ ID NO:125.

63. The epigenetic-modifying DNA-targeting system of claim 57, wherein the dCas9 protein is a Streptococcus pyogenes dCas9 (dSpCas9) protein.

64. The epigenetic-modifying DNA-targeting system of claim 63, wherein the dSpCas9 protein comprises at least one amino acid mutation selected from D10A and H840A, with reference to numbering of positions of SEQ ID NO:126.

65. The epigenetic-modifying DNA-targeting system of claim 63 or 64, wherein the dSpCas9 comprises the sequence set forth in SEQ ID NO:127, or an amino acid sequence that has at least 90%, 91%, 92%, 93%, 94%, 95%, 96%, 97%, 98%, or 99% sequence identity thereto.

66. The epigenetic-modifying DNA-targeting system of any of claims 63-65, wherein the dSpCas9 is set forth in SEQ ID NO:127.

67. The epigenetic-modifying DNA-targeting system of any of claims 54-66, wherein the gRNA comprises a gRNA spacer that is complementary to the target site of the gene.

68. The epigenetic-modifying DNA-targeting system of any of claims 54-67, wherein the DNA-targeting module is for repressing transcription of the one or more genes and the gRNA is selected from:
(a) a gRNA targeting a target site for CD5 and comprising a gRNA spacer sequence comprising the sequence set forth in any one of SEQ ID NOS:35-37, or a contiguous portion thereof of at least 14 nt;
(b) a gRNA targeting a target site for KDM1A and comprising a gRNA spacer sequence comprising the sequence set forth in any one of SEQ ID NOS:38-40, or a contiguous portion thereof of at least 14 nt;
(c) a gRNA targeting a target site for CBLB and comprising a gRNA spacer sequence comprising the sequence set forth in any one of SEQ ID NOS:44-46, or a contiguous portion thereof of at least 14 nt;
(d) a gRNA targeting a target site for DGKZ and comprising a gRNA spacer sequence comprising the sequence set forth in any one of SEQ ID NOS:47-49, or a contiguous portion thereof of at least 14 nt;
(e) a gRNA targeting a target site for MYB and comprising a gRNA spacer sequence comprising the sequence set forth in any one of SEQ ID NOS:50-52, or a contiguous portion thereof of at least 14 nt;
(f) a gRNA targeting a target site for RASA2 and comprising a gRNA spacer sequence comprising the sequence set forth in any one of SEQ ID NOS:53-55, or a contiguous portion thereof of at least 14 nt;
(g) a gRNA targeting a target site for ELOB and comprising a gRNA spacer sequence comprising the sequence set forth in any one of SEQ ID NOS:56-58, or a contiguous portion thereof of at least 14 nt;
(h) a gRNA targeting a target site for GATA3 and comprising a gRNA spacer sequence comprising the sequence set forth in any one of SEQ ID NOS:59-61, or a contiguous portion thereof of at least 14 nt;
(i) a gRNA targeting a target site for CISH and comprising a gRNA spacer sequence comprising the sequence set forth in any one of SEQ ID NOS:62-64, or a contiguous portion thereof of at least 14 nt;
(j) a gRNA targeting a target site for PRDM1 and comprising a gRNA spacer sequence comprising the sequence set forth in any one of SEQ ID NOS:65-67, or a contiguous portion thereof of at least 14 nt;
(k) a gRNA targeting a target site for MED12 and comprising a gRNA spacer sequence comprising the sequence set forth in any one of SEQ ID NOS:91-101, or a contiguous portion thereof of at least 14 nt;
(l) a gRNA targeting a target site for CCNC and comprising a gRNA spacer sequence comprising the sequence set forth in any one of SEQ ID NOS:113-123, or a contiguous portion thereof of at least 14 nt;
(m) a gRNA targeting a target site for FAS and comprising a gRNA spacer sequence comprising the sequence set forth in any one of SEQ ID NOS:212-217 and 296-299, or a contiguous portion thereof of at least 14 nt;
(n) a gRNA targeting a target site for Fli1 and comprising a gRNA spacer sequence comprising the sequence set forth in any one of SEQ ID NOS:218-223, or a contiguous portion thereof of at least 14 nt; and
(o) a gRNA targeting a target site for TGFBR2 and comprising a gRNA spacer sequence comprising the sequence set forth in any one of SEQ ID NOS:303-305 and 309-311, or a contiguous portion thereof of at least 14 nt.

69. The epigenetic-modifying DNA-targeting system of any of claims 54-68, wherein the DNA-targeting module is for repressing transcription of the one or more genes and the gRNA is selected from:
(a) a gRNA targeting a target site for CD5 and comprising a gRNA spacer sequence set forth in any one of SEQ ID NOS:35-37;
(b) a gRNA targeting a target site for KDM1A and comprising a gRNA spacer sequence set forth in any one of SEQ ID NOS:38-40;
(c) a gRNA targeting a target site for CBLB and comprising a gRNA spacer sequence set forth in any one of SEQ ID NOS:44-46;
(d) a gRNA targeting a target site for DGKZ and comprising a gRNA spacer sequence set forth in any one of SEQ ID NOS:47-49;
(e) a gRNA targeting a target site for MYB and comprising a gRNA spacer sequence set forth in any one of SEQ ID NOS:50-52;
(f) a gRNA targeting a target site for RASA2 and comprising a gRNA spacer sequence set forth in any one of SEQ ID NOS:53-55;
(g) a gRNA targeting a target site for ELOB and comprising a gRNA spacer sequence set forth in any one of SEQ ID NOS:56-58;
(h) a gRNA targeting a target site for GATA3 and comprising a gRNA spacer sequence set forth in any one of SEQ ID NOS:59-61;
(i) a gRNA targeting a target site for CISH and comprising a gRNA spacer sequence set forth in any one of SEQ ID NOS:62-64;
(j) a gRNA targeting a target site for PRDM1 and comprising a gRNA spacer sequence set forth in any one of SEQ ID NOS:65-67;
(k) a gRNA targeting a target site for MED12 and comprising a gRNA spacer sequence set forth in any one of SEQ ID NOS:91-101;
(l) a gRNA targeting a target site for CCNC and comprising a gRNA spacer sequence set forth in any one of SEQ ID NOS:113-123;
(m) a gRNA targeting a target site for FAS and comprising a gRNA spacer sequence set forth in any one of SEQ ID NOS:212-217 and 296-299;
(n) a gRNA targeting a target site for Fli1 and comprising a gRNA spacer sequence set forth in any one of SEQ ID NOS:218-223; and
(o) a gRNA targeting a target site for TGFBR2 and comprising a gRNA spacer sequence set forth in any one of SEQ ID NOS:303-305 and 309-311.

70. The epigenetic-modifying DNA-targeting system of any of claims 54-68, wherein the DNA-targeting module is for repressing transcription of the one or more genes and the gRNA is selected from:
(a) a gRNA targeting a target site for CBLB and comprising a gRNA spacer sequence set forth in SEQ ID NO:45;
(b) a gRNA targeting a target site for MYB and comprising a gRNA spacer sequence set forth in SEQ ID NO:52;
(c) a gRNA targeting a target site for RASA2 and comprising a gRNA spacer sequence set forth in SEQ ID NO:53;
(d) a gRNA targeting a target site for CISH and comprising a gRNA spacer sequence set forth in SEQ ID NO:62;
(e) a gRNA targeting a target site for PRDM1 and comprising a gRNA spacer sequence set forth in SEQ ID NO:67; and
(f) a gRNA targeting a target site for MED12 and comprising a gRNA spacer sequence set forth in SEQ ID NO:92.

71. The epigenetic-modifying DNA-targeting system of any of claims 54-67, wherein the DNA-targeting module is for increasing transcription of the one or more genes and the gRNA is selected from:
(a) a gRNA targeting a target site for VAV1 and comprising a gRNA spacer sequence comprising the sequence set forth in any one of SEQ ID NOS:41-43, 169, and 171, or a contiguous portion thereof of at least 14 nt;
(b) a gRNA targeting a target site for IL2 and comprising a gRNA spacer sequence comprising the sequence set forth in SEQ ID NO:79, or a contiguous portion thereof of at least 14 nt;
(c) a gRNA targeting a target site for BATF and comprising a gRNA spacer sequence comprising the sequence set forth in any one of SEQ ID NOS:178-180, or a contiguous portion thereof of at least 14 nt;
(d) a gRNA targeting a target site for CD28 and comprising a gRNA spacer sequence comprising the sequence set forth in any one of SEQ ID NOS:157-159 and 197-199, or a contiguous portion thereof of at least 14 nt;
(e) a gRNA targeting a target site for EOMES and comprising a gRNA spacer sequence comprising the sequence set forth in any one of SEQ ID NOS:160-162, or a contiguous portion thereof of at least 14 nt;
(f) a gRNA targeting a target site for IRF4 and comprising a gRNA spacer sequence comprising the sequence set forth in any one of SEQ ID NOS:181-183, or a contiguous portion thereof of at least 14 nt;
(g) a gRNA targeting a target site for LAT and comprising a gRNA spacer sequence comprising the sequence set forth in any one of SEQ ID NOS:192-194, or a contiguous portion thereof of at least 14 nt;
(h) a gRNA targeting a target site for LCP2 and comprising a gRNA spacer sequence comprising the sequence set forth in any one of SEQ ID NOS:163-165 and 195-196, or a contiguous portion thereof of at least 14 nt; and
(i) a gRNA targeting a target site for TBX21 and comprising a gRNA spacer sequence comprising the sequence set forth in any one of SEQ ID NOS:166-168, or a contiguous portion thereof of at least 14 nt.

72. The epigenetic-modifying DNA-targeting system of any of claims 54-67 and 71, wherein the DNA-targeting module is for increasing transcription of the one or more genes and the gRNA is selected from:
(a) a gRNA targeting a target site for VAV1 and comprising a gRNA spacer sequence set forth in any one of SEQ ID NOS:41-43, 169, and 171;
(b) a gRNA targeting a target site for IL2 and comprising a gRNA spacer sequence set forth in SEQ ID NO:79;
(c) a gRNA targeting a target site for BATF and comprising a gRNA spacer sequence set forth in any one of SEQ ID NOS:178-180;
(d) a gRNA targeting a target site for CD28 and comprising a gRNA spacer sequence set forth in any one of SEQ ID NOS:157-159 and 197-199;
(e) a gRNA targeting a target site for EOMES and comprising a gRNA spacer sequence set forth in any one of SEQ ID NOS:160-162;
(f) a gRNA targeting a target site for IRF4 and comprising a gRNA spacer sequence set forth in any one of SEQ ID NOS:181-183;
(g) a gRNA targeting a target site for LAT and comprising a gRNA spacer sequence set forth in any one of SEQ ID NOS:192-194;
(h) a gRNA targeting a target site for LCP2 and comprising a gRNA spacer sequence set forth in any one of SEQ ID NOS:163-165 and 195-196; and
(i) a gRNA targeting a target site for TBX21 and comprising a gRNA spacer sequence set forth in any one of SEQ ID NOS:166-168.

73. The epigenetic-modifying DNA-targeting system of any of claims 54-67 and 71, wherein the DNA-targeting module is for increasing transcription of the one or more genes and the gRNA is selected from:
(a) a gRNA targeting a target site for IL-2 and comprising a gRNA spacer sequence set forth in SEQ ID NO:79;
(a) a gRNA targeting a target site for EOMES and comprising a gRNA spacer sequence set forth in SEQ ID NO:162;
(a) a gRNA targeting a target site for LCP2 and comprising a gRNA spacer sequence set forth in SEQ ID NO:164; and
(a) a gRNA targeting a target site for TBX21 and comprising a gRNA spacer sequence set forth in SEQ ID NO:168.

74. The epigenetic-modifying DNA-targeting system of any of claims 54-72, wherein the gRNA comprises a spacer sequence between 14 nt and 24 nt, or between 16 nt and 22 nt in length.

75. The epigenetic-modifying DNA-targeting system of any of claims 54-74, wherein the gRNA comprises a spacer sequence that is 18 nt, 19 nt, 20 nt, 21 nt, or 22 nt in length.

76. The epigenetic-modifying DNA-targeting system of any of claims 54-75, wherein the gRNA further comprises a scaffold sequence set forth in SEQ ID NO:69.

77. The epigenetic-modifying DNA-targeting system of any of claims 1, 3-27, 33-37, 36-41, 54-69, and 74-76, wherein the at least one transcriptional repressor effector domain is capable of reducing transcription of the one or more genes.

78. The epigenetic-modifying DNA-targeting system of any of claims 1, 3-27, 33-37, 44-49, 54-69, and 74-77, wherein the transcriptional repressor effector domain is selected from the group consisting of a KRAB domain, a DNMT3A domain, a DNMT3L domain, a DNMT3B domain, a DNMT3A-DNMT3L fusion protein domain, an ERF repressor domain, an Mxil repressor domain, a SID4X repressor domain, a Mad-SID repressor domain, an LSD1 repressor domain, an EZH2 repressor domain, a SunTag domain, or a variant or portion of any of the foregoing, or a combination of any of the foregoing.

79. The epigenetic-modifying DNA-targeting system of any of claims 1, 3-27, 33-37, 44-49, 54-69, and 74-78, wherein the transcriptional repressor effector domain is a KRAB domain, a DNMT3A domain, or a DNMT3L domain, or a combination of any of the foregoing.

80. The epigenetic-modifying DNA-targeting system of any of claims 1, 3-27, 33-37, 44-49, 54-69, and 74-79, wherein the at least one transcriptional repressor effector domain comprises a KRAB domain or a variant or portion thereof that exhibits transcriptional repressor activity.

81. The epigenetic-modifying DNA-targeting system of any of claims 1, 3-27, 33-37, 44-49, 54-69, and 74-80, wherein the at least one transcriptional repressor effector domain comprises the sequence set forth in any one of SEQ ID NOS:70, 235, and 355-358, a portion thereof, or an amino acid sequence that has at least 90%, 91%, 92%, 93%, 94%, 95%, 96%, 97%, 98%, or 99% sequence identity to any of the foregoing.

82. The epigenetic-modifying DNA-targeting system of any of claims 1, 3-27, 33-37, 44-49, 54-69, and 74-79 wherein the at least one transcriptional repressor effector domain comprises a DNMT3A domain or a variant or portion thereof that exhibits transcriptional repressor activity.

83. The epigenetic-modifying DNA-targeting system of any of claims 1, 3-27, 33-37, 44-49, 54-69, 74-79 and 82, wherein the at least one transcriptional repressor domain comprises the sequence set forth in SEQ ID NO:131 or 238, a portion thereof, or an amino acid sequence that has at least 90%, 91%, 92%, 93%, 94%, 95%, 96%, 97%, 98%, or 99% sequence identity to any of the foregoing.

84. The epigenetic-modifying DNA-targeting system of any of claims 1, 3-27, 33-37, 44-49, 54-69, and 74-79, wherein the at least one transcriptional repressor domain comprises a DNMT3L domain or a variant or portion thereof that exhibits transcriptional repressor activity.

85. The epigenetic-modifying DNA-targeting system of any of claims 1, 3-27, 33-37, 44-49, 54-69, and 74-79 and 84, wherein the at least one transcriptional repressor domain comprises the sequence set forth in any one of SEQ ID NOS:133 and 240-242, a portion thereof, or an amino acid sequence that has at least 90%, 91%, 92%, 93%, 94%, 95%, 96%, 97%, 98%, or 99% sequence identity to any of the foregoing.

86. The epigenetic-modifying DNA-targeting system of any of claims 1, 3-27, 33-37, 44-49, 54-69, and 74-79, wherein the at least one transcriptional repressor domain is a DNMT3A-DNMT3L fusion protein domain, a DNMT3B-DNMT3L fusion protein domain, or a variant thereof that exhibits transcriptional repressor activity.

87. The epigenetic-modifying DNA-targeting system of any of claims 1, 3-27, 33-37, 44-49, 54-69, 74-79 and 86 wherein the at least one transcriptional repressor domain comprises the sequence set forth in any one of SEQ ID NOS:135, 137, or 363, a portion thereof, or an amino acid sequence that has at least 90%, 91%, 92%, 93%, 94%, 95%, 96%, 97%, 98%, or 99% sequence identity to any of the foregoing.

88. The epigenetic-modifying DNA-targeting system of any of claims 1, 3-27, 33-37, 44-49, 54-69, and 74-87, wherein the fusion protein comprises the sequence set forth in any one of SEQ ID NOS:138-141, 332-351, and 365-384, a portion thereof, or an amino acid sequence that has at least 90%, 91%, 92%, 93%, 94%, 95%, 96%, 97%, 98%, or 99% sequence identity thereto.

89. The DNA-targeting system of any of claims 2-24, 29, 31, 38-47, 51-67, and 71-76, wherein the at least one transcriptional activator effector domain is capable of increasing transcription of the one or more genes.

90. The DNA-targeting system of any of claims 2-24, 29, 31, 38-47, 51-67, 71-76, and 89, wherein the at least one transcriptional activator effector domain is selected from the group consisting of: a VP64 domain, a p65 activation domain, a p300 domain, an Rta domain, a CBP domain, a VPR domain, a VPH domain, an HSF1 domain, a TET protein domain, optionally wherein the TET protein is TET1, a SunTag domain, or a domain, portion, variant, or truncation of any of the foregoing.

91. The DNA-targeting system of any of claims 2-24, 29, 31, 38-47, 51-67, 71-76, 89 and 90, wherein the at least one transcriptional activator effector domain comprises at least one VP16 domain, and/or a VP16 tetramer (“VP64”) or a variant thereof.

92. The DNA-targeting system of any of claims 2-24, 29, 31, 38-47, 51-67, 71-76, and 89-91, wherein the at least one transcriptional activator effector domain comprises a VP64 domain or a variant or portion thereof that exhibits transcriptional activation activity.

93. The DNA-targeting system of any of claims 2-24, 29, 31, 38-47, 51-67, 71-76, and 89-92, wherein the at least one transcriptional activator effector domain is VP64.

94. The DNA-targeting system of any of claims 2-24, 29, 31, 38-47, 51-67, 71-76, and 89-93, wherein the at least one transcriptional activator effector domain comprises the sequence set forth in SEQ ID NO: 142, a portion thereof, or an amino acid sequence that has at least 90%, 91%, 92%, 93%, 94%, 95%, 96%, 97%, 98%, or 99% sequence identity to any of the foregoing.

95. The DNA-targeting system of any of claims 2-24, 29, 31, 38-47, 51-67, 71-76, and 89-94, wherein the fusion protein comprises the sequence set forth in SEQ ID NO:77, or an amino acid sequence that has at least 90%, 91%, 92%, 93%, 94%, 95%, 96%, 97%, 98%, or 99% sequence identity thereto.

96. A combination of epigenetic-modifying DNA-targeting systems comprising at least two of the DNA-targeting systems of any of claims 1, 3-27, 33-37, 44-49, 54-69, and 74-88, wherein each DNA-targeting system represses transcription of a different gene of the one or more genes.

97. A combination of epigenetic-modifying DNA-targeting systems comprising at least two of the DNA-targeting systems of any of claims 2-24, 29, 31, 38-47, 51-67, 71-76, and 89-95, wherein each DNA-targeting system increases transcription of a different gene of the one or more genes.

98. A guide RNA (gRNA) that targets a target site for a gene selected from the group consisting of CBLB, CCNC, CD5, CISH, DGKZ, ELOB, FAS, Fli1, GATA3, KDM1A, MED12, MYB, PRDM1, TGFBR2, and RASA2.

99. The gRNA of claim 98, wherein the target site for the gene is in the gene or a regulatory DNA element thereof.

100. The gRNA of claim 99, wherein the regulatory DNA element is an enhancer or a promoter.

101. The gRNA of any of claims 98-100, wherein the target site is within 1000 base pairs (bp) of a transcriptional start site of the gene.

102. The gRNA of any of claims 98-101, wherein the target site is within 500 base pairs (bp) of a transcriptional start site of the gene.

103. The gRNA of any of claims 98-102, wherein the target site is selected from:
(a) a target site for CD5 having the sequence set forth in any one of SEQ ID NOS:1-3, a contiguous portion thereof of at least 14 nucleotides (nt), or a complementary sequence of any of the foregoing;
(b) a target site for KDM1A having the sequence set forth in any one of SEQ ID NOS:4-6, a contiguous portion thereof of at least 14 nucleotides (nt), or a complementary sequence of any of the foregoing;
(c) a target site for CBLB having the sequence set forth in any one of SEQ ID NOS:10-12, a contiguous portion thereof of at least 14 nucleotides (nt), or a complementary sequence of any of the foregoing;
(d) a target site for DGKZ having the sequence set forth in any one of SEQ ID NOS:13-15, a contiguous portion thereof of at least 14 nucleotides (nt), or a complementary sequence of any of the foregoing;
(e) a target site for MYB having the sequence set forth in any one of SEQ ID NOS:16-18, a contiguous portion thereof of at least 14 nucleotides (nt), or a complementary sequence of any of the foregoing;
(f) a target site for RASA2 having the sequence set forth in any one of SEQ ID NOS:19-21, a contiguous portion thereof of at least 14 nucleotides (nt), or a complementary sequence of any of the foregoing;
(g) a target site for ELOB having the sequence set forth in any one of SEQ ID NOS:22-24, a contiguous portion thereof of at least 14 nucleotides (nt), or a complementary sequence of any of the foregoing;
(h) a target site for GATA3 having the sequence set forth in any one of SEQ ID NOS:25-27, a contiguous portion thereof of at least 14 nucleotides (nt), or a complementary sequence of any of the foregoing;
(i) a target site for CISH having the sequence set forth in any one of SEQ ID NOS:28-30, a contiguous portion thereof of at least 14 nucleotides (nt), or a complementary sequence of any of the foregoing;
(j) a target site for PRDM1 having the sequence set forth in any one of SEQ ID NOS:31-33, a contiguous portion thereof of at least 14 nucleotides (nt), or a complementary sequence of any of the foregoing;
(k) a target site for MED12 having the sequence set forth in any one of SEQ ID NOS:80-90, a contiguous portion thereof of at least 14 nucleotides (nt), or a complementary sequence of any of the foregoing;
(l) a target site for CCNC having the sequence set forth in any one of SEQ ID NOS:102-112, a contiguous portion thereof of at least 14 nucleotides (nt), or a complementary sequence of any of the foregoing;
(m) a target site for FAS having the sequence set forth in any one of SEQ ID NOS:200-205 and 292-295, a contiguous portion thereof of at least 14 nucleotides (nt), or a complementary sequence of any of the foregoing;
(n) a target site for Fli1 having the sequence set forth in any one of SEQ ID NOS:206-211, a contiguous portion thereof of at least 14 nucleotides (nt), or a complementary sequence of any of the foregoing; and
(o) a target site for TGFBR2 having the sequence set forth in any one of SEQ ID NOS:300-302 and 306-308, a contiguous portion thereof of at least 14 nucleotides (nt), or a complementary sequence of any of the foregoing.

104. The gRNA of any of claims 98-103, wherein the target site is selected from:
(a) a target site for CD5 having the sequence set forth in any one of SEQ ID NOS:1-3 or a complementary sequence thereof;
(b) a target site for KDM1A having the sequence set forth in any one of SEQ ID NOS:4-6 or a complementary sequence thereof;
(c) a target site for CBLB having the sequence set forth in any one of SEQ ID NOS:10-12 or a complementary sequence thereof;
(d) a target site for DGKZ having the sequence set forth in any one of SEQ ID NOS:13-15 or a complementary sequence of any of the foregoing;
(e) a target site for MYB having the sequence set forth in any one of SEQ ID NOS:16-18 or a complementary sequence of any of the foregoing;
(f) a target site for RASA2 having the sequence set forth in any one of SEQ ID NOS:19-21 or a complementary sequence of any of the foregoing;
(g) a target site for ELOB having the sequence set forth in any one of SEQ ID NOS:22-24 or a complementary sequence of any of the foregoing;
(h) a target site for GATA3 having the sequence set forth in any one of SEQ ID NOS:25-27 or a complementary sequence of any of the foregoing;
(i) a target site for CISH having the sequence set forth in any one of SEQ ID NOS:28-30 or a complementary sequence of any of the foregoing;
(j) a target site for PRDM1 having the sequence set forth in any one of SEQ ID NOS:31-33 or a complementary sequence of any of the foregoing;
(k) a target site for MED12 having the sequence set forth in any one of SEQ ID NOS:80-90 or a complementary sequence of any of the foregoing;
(l) a target site for CCNC having the sequence set forth in any one of SEQ ID NOS:102-112 or a complementary sequence of any of the foregoing;
(m) a target site for FAS having the sequence set forth in any one of SEQ ID NOS:200-205 and 292-295 or a complementary sequence of any of the foregoing;
(n) a target site for Fli1 having the sequence set forth in any one of SEQ ID NOS:206-211 or a complementary sequence of any of the foregoing; and
(o) a target site for TGFBR2 having the sequence set forth in any one of SEQ ID NOS:300-302 and 306-308 or a complementary sequence of any of the foregoing.

105. The gRNA of any of claims 98-104, wherein the target site is selected from:
(a) a target site for CBLB having the sequence set forth in SEQ ID NO:11 or a complementary sequence of any of the foregoing;
(b) a target site for MYB having the sequence set forth in SEQ ID NO:18 or a complementary sequence of any of the foregoing.
(c) a target site for RASA2 having the sequence set forth in SEQ ID NO:19 or a complementary sequence of any of the foregoing;
(d) a target site for CISH having the sequence set forth in SEQ ID NO:28 or a complementary sequence of any of the foregoing;
(e) a target site for PRDM1 having the sequence set forth in SEQ ID NO:33 or a complementary sequence of any of the foregoing; and
(f) a target site for MED12 having the sequence set forth in SEQ ID NO:81 or a complementary sequence of any of the foregoing.

106. The gRNA of any of claims 98-105, wherein the gRNA is selected from:
(a) a gRNA targeting a target site for CD5 and comprising a gRNA spacer sequence comprising the sequence set forth in any one of SEQ ID NOS:35-37, or a contiguous portion thereof of at least 14 nt;
(b) a gRNA targeting a target site for KDM1A and comprising a gRNA spacer sequence comprising the sequence set forth in any one of SEQ ID NOS:38-40, or a contiguous portion thereof of at least 14 nt;
(c) a gRNA targeting a target site for CBLB and comprising a gRNA spacer sequence comprising the sequence set forth in any one of SEQ ID NOS:44-46, or a contiguous portion thereof of at least 14 nt;
(d) a gRNA targeting a target site for DGKZ and comprising a gRNA spacer sequence comprising the sequence set forth in any one of SEQ ID NOS:47-49, or a contiguous portion thereof of at least 14 nt;
(e) a gRNA targeting a target site for MYB and comprising a gRNA spacer sequence comprising the sequence set forth in any one of SEQ ID NOS:50-52, or a contiguous portion thereof of at least 14 nt;
(f) a gRNA targeting a target site for RASA2 and comprising a gRNA spacer sequence comprising the sequence set forth in any one of SEQ ID NOS:53-55, or a contiguous portion thereof of at least 14 nt;
(g) a gRNA targeting a target site for ELOB and comprising a gRNA spacer sequence comprising the sequence set forth in any one of SEQ ID NOS:56-58, or a contiguous portion thereof of at least 14 nt;
(h) a gRNA targeting a target site for GATA3 and comprising a gRNA spacer sequence comprising the sequence set forth in any one of SEQ ID NOS:59-61, or a contiguous portion thereof of at least 14 nt;
(i) a gRNA targeting a target site for CISH and comprising a gRNA spacer sequence comprising the sequence set forth in any one of SEQ ID NOS:62-64, or a contiguous portion thereof of at least 14 nt;
(j) a gRNA targeting a target site for PRDM1 and comprising a gRNA spacer sequence comprising the sequence set forth in any one of SEQ ID NOS:65-67, or a contiguous portion thereof of at least 14 nt;
(k) a gRNA targeting a target site for MED12 and comprising a gRNA spacer sequence comprising the sequence set forth in any one of SEQ ID NOS:91-101, or a contiguous portion thereof of at least 14 nt;
(l) a gRNA targeting a target site for CCNC and comprising a gRNA spacer sequence comprising the sequence set forth in any one of SEQ ID NOS:113-123, or a contiguous portion thereof of at least 14 nt;
(m) a gRNA targeting a target site for FAS and comprising a gRNA spacer sequence comprising the sequence set forth in any one of SEQ ID NOS:212-217 and 296-299, or a contiguous portion thereof of at least 14 nt;
(n) a gRNA targeting a target site for Fli1 and comprising a gRNA spacer sequence comprising the sequence set forth in any one of SEQ ID NOS:218-223, or a contiguous portion thereof of at least 14 nt; and
(o) a gRNA targeting a target site for TGFBR2 and comprising a gRNA spacer sequence comprising the sequence set forth in any one of SEQ ID NOS:303-305 and 309-311, or a contiguous portion thereof of at least 14 nt.

107. The gRNA of any of claims 98-106, wherein the gRNA is selected from:
(a) a gRNA targeting a target site for CD5 and comprising a gRNA spacer sequence set forth in any one of SEQ ID NOS:35-37;
(b) a gRNA targeting a target site for KDM1A and comprising a gRNA spacer sequence set forth in any one of SEQ ID NOS:38-40;
(c) a gRNA targeting a target site for CBLB and comprising a gRNA spacer sequence set forth in any one of SEQ ID NOS:44-46;
(d) a gRNA targeting a target site for DGKZ and comprising a gRNA spacer sequence set forth in any one of SEQ ID NOS:47-49;
(e) a gRNA targeting a target site for MYB and comprising a gRNA spacer sequence set forth in any one of SEQ ID NOS:50-52;
(f) a gRNA targeting a target site for RASA2 and comprising a gRNA spacer sequence set forth in any one of SEQ ID NOS:53-55;
(g) a gRNA targeting a target site for ELOB and comprising a gRNA spacer sequence set forth in any one of SEQ ID NOS:56-58;
(h) a gRNA targeting a target site for GATA3 and comprising a gRNA spacer sequence set forth in any one of SEQ ID NOS:59-61;
(i) a gRNA targeting a target site for CISH and comprising a gRNA spacer sequence set forth in any one of SEQ ID NOS:62-64;
(j) a gRNA targeting a target site for PRDM1 and comprising a gRNA spacer sequence set forth in any one of SEQ ID NOS:65-67;
(k) a gRNA targeting a target site for MED12 and comprising a gRNA spacer sequence set forth in any one of SEQ ID NOS:91-101;
(l) a gRNA targeting a target site for CCNC and comprising a gRNA spacer sequence set forth in any one of SEQ ID NOS:113-123;
(m) a gRNA targeting a target site for FAS and comprising a gRNA spacer sequence set forth in any one of SEQ ID NOS:212-217 and 296-299;
(n) a gRNA targeting a target site for Fli1 and comprising a gRNA spacer sequence set forth in any one of SEQ ID NOS:218-223; and
(o) a gRNA targeting a target site for TGFBR2 and comprising a gRNA spacer sequence set forth in any one of SEQ ID NOS:303-305 and 309-311.

108. The gRNA of any of claims 98-107, wherein the gRNA is selected from:
(a) a gRNA targeting a target site for CBLB and comprising a gRNA spacer sequence set forth in SEQ ID NO:45;
(b) a gRNA targeting a target site for MYB and comprising a gRNA spacer sequence set forth in SEQ ID NO:52;
(c) a gRNA targeting a target site for RASA2 and comprising a gRNA spacer sequence set forth in SEQ ID NO:53;
(d) a gRNA targeting a target site for CISH and comprising a gRNA spacer sequence set forth in SEQ ID NO:62;
(e) a gRNA targeting a target site for PRDM1 and comprising a gRNA spacer sequence set forth in SEQ ID NO:67; and
(f) a gRNA targeting a target site for MED12 and comprising a gRNA spacer sequence set forth in SEQ ID NO:91.

109. The gRNA of any of claims 98-108, wherein the gRNA comprises a spacer sequence between 14 nt and 24 nt, or between 16 nt and 22 nt in length.

110. The gRNA of any of claims 98-109, wherein the gRNA comprises a spacer sequence that is 18 nt, 19 nt, 20 nt, 21 nt, or 22 nt in length.

111. The gRNA of any of claims 98-110, wherein the gRNA further comprises a scaffold sequence set forth in SEQ ID NO:69.

112. A guide RNA (gRNA) that targets a target site for a gene selected from the group consisting of BATF, CD28, EOMES, IL-2, IL2RB, IRF4, LAT, LCP2, TBX21, and VAV1.

113. The gRNA of claim 112, wherein the target site for the gene is in the gene or a regulatory DNA element thereof.

114. The gRNA of claim 113, wherein the regulatory DNA element is an enhancer or a promoter.

115. The gRNA of any of claims 112-114, wherein the target site is within 1000 base pairs (bp) of a transcriptional start site of the gene.

116. The gRNA of any of claims 112-115, wherein the target site is within 500 base pairs (bp) of a transcriptional start site of the gene.

117. The gRNA of any of claims 112-116, wherein the target site is selected from:
(a) a target site for VAV1 having the sequence set forth in any one of SEQ ID NOS:7-9, 156, and 170, a contiguous portion thereof of at least 14 nucleotides (nt), or a complementary sequence of any of the foregoing;
(b) a target site for IL2 having the sequence set forth in SEQ ID NO:78, a contiguous portion thereof of at least 14 nucleotides (nt), or a complementary sequence of any of the foregoing;
(c) a target site for BATF having the sequence set forth in any one of SEQ ID NOS:172-174, a contiguous portion thereof of at least 14 nucleotides (nt), or a complementary sequence of any of the foregoing;
(d) a target site for CD28 having the sequence set forth in any one of SEQ ID NOS:144-146 and 189-191, a contiguous portion thereof of at least 14 nucleotides (nt), or a complementary sequence of any of the foregoing;
(e) a target site for EOMES having the sequence set forth in any one of SEQ ID NOS:147-149, a contiguous portion thereof of at least 14 nucleotides (nt), or a complementary sequence of any of the foregoing;
(f) a target site for IRF4 having the sequence set forth in any one of SEQ ID NOS:175-177, a contiguous portion thereof of at least 14 nucleotides (nt), or a complementary sequence of any of the foregoing;
(g) a target site for LAT having the sequence set forth in any one of SEQ ID NOS:184-186, a contiguous portion thereof of at least 14 nucleotides (nt), or a complementary sequence of any of the foregoing;
(h) a target site for LCP2 having the sequence set forth in any one of SEQ ID NOS:150-152 and 187-188, a contiguous portion thereof of at least 14 nucleotides (nt), or a complementary sequence of any of the foregoing; and
(i) a target site for TBX21 having the sequence set forth in any one of SEQ ID NOS:153-155, a contiguous portion thereof of at least 14 nucleotides (nt), or a complementary sequence of any of the foregoing.

118. The gRNA of any of claims 112-117, wherein the target site is selected from:
(a) a target site for VAV1 having the sequence set forth in any one of SEQ ID NOS:7-9, 156, and 170, or a complementary sequence of any of the foregoing;
(b) a target site for IL2 having the sequence set forth in SEQ ID NO:78 or a complementary sequence of any of the foregoing;
(c) a target site for BATF having the sequence set forth in any one of SEQ ID NOS:172-174, or a complementary sequence of any of the foregoing;
(d) a target site for CD28 having the sequence set forth in any one of SEQ ID NOS:144-146 and 189-191, or a complementary sequence of any of the foregoing;
(e) a target site for EOMES having the sequence set forth in any one of SEQ ID NOS:147-149, or a complementary sequence of any of the foregoing;
(f) a target site for IRF4 having the sequence set forth in any one of SEQ ID NOS:175-177, or a complementary sequence of any of the foregoing;
(g) a target site for LAT having the sequence set forth in any one of SEQ ID NOS:184-186, or a complementary sequence of any of the foregoing;
(h) a target site for LCP2 having the sequence set forth in any one of SEQ ID NOS:150-152 and 187-188, or a complementary sequence of any of the foregoing; and
(i) a target site for TBX21 having the sequence set forth in any one of SEQ ID NOS:153-155, or a complementary sequence of any of the foregoing.

119. The gRNA of any of claims 112-118, wherein the target site is selected from:
(a) a target site for IL-2 having the sequence set forth in SEQ ID NO:78, or a complementary sequence of any of the foregoing;
(b) a target site for EOMES having the sequence set forth in SEQ ID NO:149, or a complementary sequence of any of the foregoing;
(c) a target site for LCP2 having the sequence set forth in SEQ ID NO:151, or a complementary sequence of any of the foregoing; and
(d) a target site for TBX21 having the sequence set forth in SEQ ID NO:155, or a complementary sequence of any of the foregoing.

120. The gRNA of any of claims 112-119, wherein the gRNA is selected from:
(a) a gRNA targeting a target site for VAV1 and comprising a gRNA spacer sequence comprising the sequence set forth in any one of SEQ ID NOS:41-43, 169, and 171, or a contiguous portion thereof of at least 14 nt;
(b) a gRNA targeting a target site for IL2 and comprising a gRNA spacer sequence comprising the sequence set forth in SEQ ID NO:79, or a contiguous portion thereof of at least 14 nt;
(c) a gRNA targeting a target site for BATF and comprising a gRNA spacer sequence comprising the sequence set forth in any one of SEQ ID NOS:178-180, or a contiguous portion thereof of at least 14 nt;
(d) a gRNA targeting a target site for CD28 and comprising a gRNA spacer sequence comprising the sequence set forth in any one of SEQ ID NOS:157-159 and 197-199, or a contiguous portion thereof of at least 14 nt;
(e) a gRNA targeting a target site for EOMES and comprising a gRNA spacer sequence comprising the sequence set forth in any one of SEQ ID NOS:160-162, or a contiguous portion thereof of at least 14 nt;
(f) a gRNA targeting a target site for IRF4 and comprising a gRNA spacer sequence comprising the sequence set forth in any one of SEQ ID NOS:181-183, or a contiguous portion thereof of at least 14 nt;
(g) a gRNA targeting a target site for LAT and comprising a gRNA spacer sequence comprising the sequence set forth in any one of SEQ ID NOS:192-194, or a contiguous portion thereof of at least 14 nt;
(h) a gRNA targeting a target site for LCP2 and comprising a gRNA spacer sequence comprising the sequence set forth in any one of SEQ ID NOS:163-165 and 195-196, or a contiguous portion thereof of at least 14 nt; and
(i) a gRNA targeting a target site for TBX21 and comprising a gRNA spacer sequence comprising the sequence set forth in any one of SEQ ID NOS:166-168, or a contiguous portion thereof of at least 14 nt.

121. The gRNA of any of claims 112-120, wherein the gRNA is selected from:
(a) a gRNA targeting a target site for VAV1 and comprising a gRNA spacer sequence set forth in any one of SEQ ID NOS:41-43, 169, and 171;
(b) a gRNA targeting a target site for IL2 and comprising a gRNA spacer sequence set forth in SEQ ID NO:79;
(c) a gRNA targeting a target site for BATF and comprising a gRNA spacer sequence set forth in any one of SEQ ID NOS:178-180;
(d) a gRNA targeting a target site for CD28 and comprising a gRNA spacer sequence set forth in any one of SEQ ID NOS:157-159 and 197-199;
(e) a gRNA targeting a target site for EOMES and comprising a gRNA spacer sequence set forth in any one of SEQ ID NOS:160-162;
(f) a gRNA targeting a target site for IRF4 and comprising a gRNA spacer sequence set forth in any one of SEQ ID NOS:181-183;
(g) a gRNA targeting a target site for LAT and comprising a gRNA spacer sequence set forth in any one of SEQ ID NOS:192-194;
(h) a gRNA targeting a target site for LCP2 and comprising a gRNA spacer sequence set forth in any one of SEQ ID NOS:163-165 and 195-196; and
(i) a gRNA targeting a target site for TBX21 and comprising a gRNA spacer sequence set forth in any one of SEQ ID NOS:166-168.

122. The gRNA of any of claims 112-121, wherein the gRNA is selected from:
(a) a gRNA targeting a target site for IL-2 and comprising a gRNA spacer sequence set forth in SEQ ID NO:79;
(b) a gRNA targeting a target site for EOMES and comprising a gRNA spacer sequence set forth in SEQ ID NO:162;
(c) a gRNA targeting a target site for LCP2 and comprising a gRNA spacer sequence set forth in SEQ ID NO:164; and
(d) a gRNA targeting a target site for TBX21 and comprising a gRNA spacer sequence set forth in SEQ ID NO:168.

123. The gRNA of any of claims 112-122, wherein the gRNA comprises a spacer sequence between 14 nt and 24 nt, or between 16 nt and 22 nt in length.

124. The gRNA of any of claims 112-123, wherein the gRNA comprises a spacer sequence that is 18 nt, 19 nt, 20 nt, 21 nt, or 22 nt in length.

125. The gRNA of any of claims 112-124, wherein the gRNA further comprises a scaffold sequence set forth in SEQ ID NO:69.

126. A combination of gRNAs comprising:
two or more gRNAs, each selected from the gRNA of any of claims 98-111, or
two or more gRNAs, each selected from the gRNA of any of claims 112-125.

127. The combination of gRNAs of claim 126,
wherein the two gRNAs comprise spacer sequences set forth in SEQ ID NOS:92 and 45; SEQ ID NOs: 92 and 62; SEQ ID NOS:45 and 52; and SEQ ID NOS:45 and 53; SEQ ID NOs: 92 and 304; SEQ ID NOs: 92, 304, or 62; or
wherein the two gRNAs comprise spacer sequences set forth in SEQ ID NOS: 79 and 164; SEQ ID NOS:79 and 168; SEQ ID NOS:79 and 162.

128. A Cas-guide RNA (gRNA) combination comprising:
(a) a Clustered Regularly Interspaced Short Palindromic Repeats associated (Cas) protein or variant thereof; and
(b) at least one gRNA of any of claims 98-111.

129. A Cas-guide RNA (gRNA) combination comprising:
(a) a Clustered Regularly Interspaced Short Palindromic Repeats associated (Cas) protein or variant thereof; and
(b) at least one gRNA of any of claims 112-125.

130. The Cas-guide RNA (gRNA) combination of claim 128 or 129, wherein the Cas protein or variant thereof is a deactivated (dCas) protein.

131. The Cas-guide RNA (gRNA) combination of claim 130, wherein the dCas protein lacks nuclease activity.

132. The Cas-guide RNA (gRNA) combination of claim 130 or 131, wherein the dCas protein is a dCas9 protein.

133. The Cas-guide RNA (gRNA) combination of claim 130 or 131, wherein the dCas protein is a dCas12 protein.

134. The Cas-guide RNA (gRNA) combination of claim 132, wherein the dCas9 protein is a Staphylococcus aureus dCas9 (dSaCas9) protein.

135. The Cas-guide RNA (gRNA) combination of claim 134, wherein the dSaCas9 comprises at least one amino acid mutation selected from D10A and N580A, with reference to numbering of positions of SEQ ID NO:124.

136. The Cas-guide RNA (gRNA) combination of claim 134 or 135, wherein the dSaCas9 protein comprises the sequence set forth in SEQ ID NO:125, or an amino acid sequence that has at least 90%, 91%, 92%, 93%, 94%, 95%, 96%, 97%, 98%, or 99% sequence identity thereto.

137. The Cas-guide RNA (gRNA) combination of any of claims 134-136, wherein the dSaCas9 is set forth in SEQ ID NO:125.

138. The Cas-guide RNA (gRNA) combination of claim 132, wherein the dCas9 protein is a Streptococcus pyogenes dCas9 (dSpCas9) protein.

139. The Cas-guide RNA (gRNA) combination of claim 138, wherein the dSpCas9 protein comprises at least one amino acid mutation selected from D10A and H840A, with reference to numbering of positions of SEQ ID NO:126.

140. The Cas-guide RNA (gRNA) combination of claim 121 or 122, wherein the dSpCas9 comprises the sequence set forth in SEQ ID NO:127, or an amino acid sequence that has at least 90%, 91%, 92%, 93%, 94%, 95%, 96%, 97%, 98%, or 99% sequence identity thereto.

141. The Cas-guide RNA (gRNA) combination of any of claims 138-140, wherein the dSpCas9 is set forth in SEQ ID NO:127.

142. A polynucleotide encoding the epigenetic-modifying DNA-targeting system of any of claims 1-95.

143. A polynucleotide encoding at least one DNA-targeting module of the epigenetic-modifying DNA-targeting system of any of claims 1-95.

144. A polynucleotide encoding the fusion protein and the at least one gRNA of the epigenetic-modifying DNA-targeting system of any of claims 1-95.

145. A polynucleotide encoding the gRNA of any of claims 98-125.

146. A polynucleotide encoding the combination of gRNAs of claim 126.

147. A polynucleotide encoding the Cas-gRNA combination of any of claims 128-141.

148. Two or more polynucleotides that together encode:
the epigenetic-modifying DNA-targeting system of any of claims 1-95,
at least one DNA-targeting module of the epigenetic-modifying DNA-targeting system of any of claims 1-95,
the fusion protein and the at least one gRNA of the epigenetic-modifying DNA-targeting system of any of claims 1-95,
the combination of gRNAs of claim 126, and/or
the Cas-gRNA combination of any of claims 128-141.

149. A polynucleotide gRNA combination comprising:
a) a polynucleotide encoding the fusion protein of at least one of the DNA-targeting modules for repressing transcription of the one or more genes of the epigenetic-modifying DNA-targeting system of any of claims 1, 3-26, 33-37, 44-49, 54-69, 74-88, and 96, and one or more gRNAs selected from the gRNA of any of claims 98-111; or
b) a polynucleotide encoding the fusion protein of at least one of the DNA-targeting modules for increasing transcription of the one or more genes of the epigenetic-modifying DNA-targeting system of any of claims 2-24, 29, 31, 38-47, 51-67, 71-76, 89-95, and 97, and one or more gRNAs selected from the gRNA of any of claims 112-125.

150. The polynucleotide gRNA combination of claim 149, wherein the polynucleotide encoding the fusion protein is mRNA.

151. A vector comprising the polynucleotide of any of claims 142-147.

152. A vector comprising the two or more polynucleotides of claim 148.

153. A vector comprising the polynucleotide gRNA combination of claim 149.

154. A vector comprising the polynucleotide gRNA combination of claim 150.

155. The vector of any of claims 151-154, wherein the vector is a viral vector.

156. The vector of claim 155, wherein the vector is an adeno-associated virus (AAV) vector.

157. The vector of claim 156, wherein the vector is selected from among AAV1, AAV2, AAV3, AAV4, AAV5, AAV6, AAV7, AAV8, and AAV9.

158. The vector of any of claims 151-154, wherein the vector is a non-viral vector.

159. The vector of claim 158, wherein the non-viral vector is selected from: a lipid nanoparticle, a liposome, an exosome, or a cell penetrating peptide.

160. The vector of claim 158 or 159, wherein the non-viral vector is a lipid nanoparticle.

161. The vector of any of claims 151-160, wherein the vector exhibits immune cell tropism, optionally wherein the vector exhibits T-cell tropism.

162. A modified T cell comprising the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, or the polynucleotide gRNA combination of claim 149 or 150.

163. A modified T cell comprising an epigenetic or phenotypic modification resulting from being contacted by the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, the polynucleotide gRNA combination of claim 149 or 150, or the vector of any of claims 151-161.

164. The modified T cell of claim 162 or 163, wherein the modified T cell is derived from a cell from a subject.

165. The modified T cell of any of claims 162-164, wherein the modified T cell is derived from a primary T cell.

166. The modified T cell of any of claims 162-165, wherein the modified T cell is derived from a T cell progenitor, a pluripotent stem cell, or an induced pluripotent stem cell.

167. The modified T cell of any of claims 162-166, wherein the modified T cell further comprises an engineered T cell receptor (eTCR) or chimeric antigen receptor (CAR).

168. A method of repressing the transcription of one or more genes in a T cell, the method comprising introducing into a T cell the DNA-targeting system of any of claims 1, 3-26, 33-37, 44-49, 54-69, and 74-88, the combination of DNA-targeting systems of claim 96, the gRNA of any of claims 98-99, the combination of gRNAs of claim 126 or claim 127, the Cas-gRNA combination of any of claims 128 and 130-145, the polynucleotide of any of claims 142-147, the two or more polynucleotides of claim 148, the polynucleotide gRNA combination of claim 149 or 150, or the vector of any of claims 151-161.

169. The method of claim 168, wherein repressing transcription of the one or more genes promotes increased T cell effector function upon T cell stimulation relative to the T cell effector function in the absence of the T cell stimulation.

170. A method of increasing the transcription of one or more genes in a T cell, the method comprising introducing into a T cell the DNA-targeting system of any of claims 2-24, 29, 31, 38-47, 51-67, 71-76, and 89-95, the combination of DNA-targeting systems of claim 97, the gRNA of any of claims 112-125, the combination of gRNAs of claim 126 or claim 127, the Cas-gRNA combination of any of claims 129 and 130-141, the polynucleotide of any of claims 142-147, the two or more polynucleotides of claim 148, the polynucleotide gRNA combination of claim 149 or 150, or the vector of any of claims 151-161.

171. The method of claim 170, wherein increasing transcription of the one or more genes promotes increased T cell effector function upon T cell stimulation relative to the T cell effector function in the absence of the T cell stimulation.

172. A method of increasing T cell effector function, the method comprising introducing into a T cell the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, the polynucleotide gRNA combination of claim 149 or 150, or the vector of any of claims 151-161.

173. The method of claim 169, 171, or 172, wherein the T cell effector function is increased compared to a T cell that has not been introduced with the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, the polynucleotide gRNA combination of claim 149 or 150, or the vector of any of claims 151-161.

174. The method of any of claims 168-173, wherein the T cell is a T cell in a subject and the method is carried out in vivo.

175. The method of any of claims 168-173, wherein the T cell is a T cell from a subject, or derived from a cell from the subject, and the method is carried out ex vivo.

176. The method of claim 175, wherein the T cell is a primary T cell.

177. The method of claim 176, wherein the T cell is derived from a T cell progenitor, a pluripotent stem cell, or an induced pluripotent stem cell.

178. The method of any of claims 168-177, wherein the introducing is by transient delivery into the T cell.

179. The method of claim 178, wherein the transient delivery comprises electroporation, transfection, or transduction.

180. The method of any of claims 168-179, wherein the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, the polynucleotide gRNA combination of claim 149 or 150, or the vector of any of claims 151-161, is transiently expressed and/or transiently present in the T cell.

181. The method of any of any of claims 168, 169, and 272-180, wherein the introducing represses transcription of one or more genes in the T cell selected from the group consisting of CBLB, CCNC, CD5, CISH, DGKZ, ELOB, FAS, Fli1, GATA3, KDM1A, MED12, MYB, PRDM1, TGFBR2, and RASA2.

182. The method of any of claims 170-180, wherein the introducing increases transcription of one or more genes in the T cells selected from the group consisting of BATF, CD28, EOMES, IL-2, IL2RB, IRF4, LAT, LCP2, TBX21, and VAV1.

183. A modified T cell produced by the method of any of claims 168-182.

184. A method of treating a disease or condition in a subject, the method comprising administering to the subject the modified T cell of any of claims 162-167 and 183.

185. A method of increasing T cell persistence in T cells of a subject, the method comprising administering to the subject or T cells thereof the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, the polynucleotide gRNA combination of claim 149 or 150, or the vector of any of claims 151-161.

186. The method of claim 185, wherein the T cell is from an adoptive T cell therapy for treating a disease or condition in the subject.

187. The method of claim 186, wherein the T cell therapy comprises T cells expressing a recombinant receptor specific for a target antigen.

188. The method of claim 186 or 187, wherein the administration is carried out prior to, concurrently with, or after administration of the adoptive T cell therapy.

189. The method of any of claims 186-188, wherein the administration is carried out after administration of the adoptive T cell therapy in the subject, at a time after the numbers or effector function of T cells of the adoptive T cell therapy are reduced, or are suspected of being reduced, in the subject.

190. A method of treating a disease or condition in a subject, the method comprising administering to a subject:
a T cell therapy comprising cells expressing a recombinant receptor specific for a target antigen associated with the disease or condition; and
the DNA-targeting system of any of claims 1-95, the combination of DNA-targeting systems of claim 96 or 97, the gRNA of any of claims 98-125, the combination of gRNAs of claim 126 or claim 127, the CRISPR Cas-gRNA combination of any of claims 128-141, the polynucleotide of any of claims 142-147, the two or more polynucleotides of claim 148, the polynucleotide gRNA combination of claim 149 or 150, or the vector of any of claims 151-161.

191. The method of any of claims 187-190, wherein the recombinant receptor is an engineered T cell receptor (eTCR) or chimeric antigen receptor (CAR).

192. The method of any of claims 187-191, wherein the target antigen is a tumor antigen.

193. The method of any of claims 186-192, wherein the disease or condition is a cancer.

194. The method of claim 193, wherein the cancer is a hematological cancer or is a solid tumor.

195. The method of any of claims 186-192, wherein the disease or condition is an autoimmune condition and/or an inflammatory condition.

196. The method of any of claims 185-195, wherein the administering results in transient delivery into the T cell: the DNA-targeting system, the combination of DNA-targeting systems, the gRNA, the combination of gRNAs, the CRISPR Cas-gRNA combination, the polynucleotide, the two or more polynucleotides, the polynucleotide gRNA combination, or the vector.

197. The method of any of any of claims 185-196, wherein the administering represses transcription of one or more genes in the T cell selected from the group consisting of CBLB, CCNC, CD5, CISH, DGKZ, ELOB, FAS, Fli1, GATA3, KDM1A, MED12, MYB, PRDM1, TGFBR2, and RASA2.

198. The method of any of any of claims 185-197, wherein the administering represses transcription of one or more genes in the T cell selected from the group consisting of CBLB, CISH, MED12, MYB, PRDM1, and RASA2.

199. The method of any of claims 185-196, wherein the administering increases transcription of one or more genes in the T cells selected from the group consisting of BATF, CD28, EOMES, IL-2, IL2RB, IRF4, LAT, LCP2, TBX21, and VAV1.

200. The method of any of claims 185-196, or 199, wherein the administering increases transcription of one or more genes in the T cells selected from the group consisting of EOMES, IL-2, LCP2, and TBX21.


SEQUENCE LISTING (424 sequences)

SEQ ID NO: 1 - DNA, 20 bp, synthetic construct
  misc_feature 1..20: note = CD5_1 target site
  source 1..20: mol_type = other DNA
ttgcactggaagggtaaagc

SEQ ID NO: 2 - DNA, 20 bp, synthetic construct
  misc_feature 1..20: note = CD5_2 target site
  source 1..20: mol_type = other DNA
ggaggcgaccaagtaaaggc

SEQ ID NO: 3 - DNA, 20 bp, synthetic construct
  misc_feature 1..20: note = CD5_3 target site
  source 1..20: mol_type = other DNA
gtcagtgggggacctcgcag

SEQ ID NO: 4 - DNA, 20 bp, synthetic construct
  misc_feature 1..20: note = KDM1A_1 target site
  source 1..20: mol_type = other DNA
cgcgcgggcagcgtgaagcg

SEQ ID NO: 5 - DNA, 20 bp, synthetic construct
  misc_feature 1..20: note = KDM1A_2 target site
  source 1..20: mol_type = other DNA
agcggcagcaaccgggacgg

SEQ ID NO: 6 - DNA, 20 bp, synthetic construct
  misc_feature 1..20: note = KDM1A_3 target site
  source 1..20: mol_type = other DNA
gcccagaagccctaagacca

SEQ ID NO: 7 - DNA, 20 bp, synthetic construct
  misc_feature 1..20: note = VAV1_1 target site
  source 1..20: mol_type = other DNA
tgtcgctccacaggcgagca

SEQ ID NO: 8 - DNA, 20 bp, synthetic construct
  misc_feature 1..20: note = VAV1_2 target site
  source 1..20: mol_type = other DNA
cctctcagggcgacagttac

SEQ ID NO: 9 - DNA, 20 bp, synthetic construct
  misc_feature 1..20: note = VAV1_3 target site
  source 1..20: mol_type = other DNA
ggccagctagactatgagat

SEQ ID NO: 10 - DNA, 20 bp, synthetic construct
  misc_feature 1..20: note = CBLB_1 target site
  source 1..20: mol_type = other DNA
gaacagctcgctcccgaaga

SEQ ID NO: 11 - DNA, 20 bp, synthetic construct
  misc_feature 1..20: note = CBLB_2 target site
  source 1..20: mol_type = other DNA
cgctgggttgctccttcttc

SEQ ID NO: 12 - DNA, 20 bp, synthetic construct
  misc_feature 1..20: note = CBLB_3 target site
  source 1..20: mol_type = other DNA
cgtccaggcagacggcggtg

SEQ ID NO: 13 - DNA, 20 bp, synthetic construct
  misc_feature 1..20: note = DGKZ_1 target site
  source 1..20: mol_type = other DNA
gggacacgggcgggatcggt

SEQ ID NO: 14 - DNA, 20 bp, synthetic construct
  misc_feature 1..20: note = DGKZ_2 target site
  source 1..20: mol_type = other DNA
ggagcgagcgcgcgccatgg

SEQ ID NO: 15 - DNA, 20 bp, synthetic construct
  misc_feature 1..20: note = DGKZ_3 target site
  source 1..20: mol_type = other DNA
tcttcgggcacaggtgagcg

SEQ ID NO: 16 - DNA, 20 bp, synthetic construct
  misc_feature 1..20: note = MYB_1 target site
  source 1..20: mol_type = other DNA
gccgaatgggagcggcgacc

SEQ ID NO: 17 - DNA, 20 bp, synthetic construct
  misc_feature 1..20: note = MYB_2 target site
  source 1..20: mol_type = other DNA
ggatccctcgccgacacccg

SEQ ID NO: 18 - DNA, 20 bp, synthetic construct
  misc_feature 1..20: note = MYB_3 target site
  source 1..20: mol_type = other DNA
gaaacttcgccccagcggtg

SEQ ID NO: 19 - DNA, 20 bp, synthetic construct
  misc_feature 1..20: note = RASA2_1 target site
  source 1..20: mol_type = other DNA
gcacgggccgggcggcacca

SEQ ID NO: 20 - DNA, 20 bp, synthetic construct
  misc_feature 1..20: note = RASA2_2 target site
  source 1..20: mol_type = other DNA
tgctgcggcggcttcttccg

SEQ ID NO: 21 - DNA, 20 bp, synthetic construct
  misc_feature 1..20: note = RASA2_3 target site
  source 1..20: mol_type = other DNA
gcgctgggcgcgaggctgag

SEQ ID NO: 22 - DNA, 20 bp, synthetic construct
  misc_feature 1..20: note = ELOB_1 target site
  source 1..20: mol_type = other DNA
cgaactccttgggctagaag

SEQ ID NO: 23 - DNA, 20 bp, synthetic construct
  misc_feature 1..20: note = ELOB_2 target site
  source 1..20: mol_type = other DNA
tgcggccgccatcccgacgg

SEQ ID NO: 24 - DNA, 20 bp, synthetic construct
  misc_feature 1..20: note = ELOB_3 target site
  source 1..20: mol_type = other DNA
ctggaagcgggcggtatcga

SEQ ID NO: 25 - DNA, 20 bp, synthetic construct
  misc_feature 1..20: note = GATA3_1 target site
  source 1..20: mol_type = other DNA
cggagggtacctctgcaccg

SEQ ID NO: 26 - DNA, 20 bp, synthetic construct
  misc_feature 1..20: note = GATA3_2 target site
  source 1..20: mol_type = other DNA
tcgacgaggaggctccaccc

SEQ ID NO: 27 - DNA, 20 bp, synthetic construct
  misc_feature 1..20: note = GATA3_3 target site
  source 1..20: mol_type = other DNA
cagggctgactgttacgact

SEQ ID NO: 28 - DNA, 20 bp, synthetic construct
  misc_feature 1..20: note = CISH_1 target site
  source 1..20: mol_type = other DNA
tgtcctgcgcccgcgcgccc

SEQ ID NO: 29 - DNA, 20 bp, synthetic construct
  misc_feature 1..20: note = CISH_2 target site
  source 1..20: mol_type = other DNA
ggcggctggagggaaccagt

SEQ ID NO: 30 - DNA, 20 bp, synthetic construct
  misc_feature 1..20: note = CISH_3 target site
  source 1..20: mol_type = other DNA
gtggcgcggaccgcctgcga

SEQ ID NO: 31 - DNA, 20 bp, synthetic construct
  misc_feature 1..20: note = PRDM1_1 target site
  source 1..20: mol_type = other DNA
agaggcaagagcagcgaccg

SEQ ID NO: 32 - DNA, 20 bp, synthetic construct
  misc_feature 1..20: note = PRDM1_2 target site
  source 1..20: mol_type = other DNA
gacgcggggagaatgtggac

SEQ ID NO: 33 - DNA, 20 bp, synthetic construct
  misc_feature 1..20: note = PRDM1_3 target site
  source 1..20: mol_type = other DNA
ttgcctctccgcaacactgg

SEQ ID NO: 34 - DNA, 20 bp, synthetic construct
  misc_feature 1..20: note = non-targeting_sp_1 target site
  source 1..20: mol_type = other DNA
acggaggctaagcgtcgcaa

SEQ ID NO: 35 - RNA, 20 nt, synthetic construct
  misc_feature 1..20: note = CD5_1 gRNA
  source 1..20: mol_type = other RNA
ttgcactggaagggtaaagc

SEQ ID NO: 36 - RNA, 20 nt, synthetic construct
  misc_feature 1..20: note = CD5_2 gRNA
  source 1..20: mol_type = other RNA
ggaggcgaccaagtaaaggc

SEQ ID NO: 37 - RNA, 20 nt, synthetic construct
  misc_feature 1..20: note = CD5_3 gRNA
  source 1..20: mol_type = other RNA
gtcagtgggggacctcgcag

SEQ ID NO: 38 - RNA, 20 nt, synthetic construct
  misc_feature 1..20: note = KDM1A_1 gRNA
  source 1..20: mol_type = other RNA
cgcgcgggcagcgtgaagcg

SEQ ID NO: 39 - RNA, 20 nt, synthetic construct
  misc_feature 1..20: note = KDM1A_2 gRNA
  source 1..20: mol_type = other RNA
agcggcagcaaccgggacgg

SEQ ID NO: 40 - RNA, 20 nt, synthetic construct
  misc_feature 1..20: note = KDM1A_3 gRNA
  source 1..20: mol_type = other RNA
gcccagaagccctaagacca

SEQ ID NO: 41 - RNA, 20 nt, synthetic construct
  misc_feature 1..20: note = VAV1_1 gRNA
  source 1..20: mol_type = other RNA
tgtcgctccacaggcgagca

SEQ ID NO: 42 - RNA, 20 nt, synthetic construct
  misc_feature 1..20: note = VAV1_2 gRNA
  source 1..20: mol_type = other RNA
cctctcagggcgacagttac

SEQ ID NO: 43 - RNA, 20 nt, synthetic construct
  misc_feature 1..20: note = VAV1_3 gRNA
  source 1..20: mol_type = other RNA
ggccagctagactatgagat

SEQ ID NO: 44 - RNA, 20 nt, synthetic construct
  misc_feature 1..20: note = CBLB_1 gRNA
  source 1..20: mol_type = other RNA
gaacagctcgctcccgaaga

SEQ ID NO: 45 - RNA, 20 nt, synthetic construct
  misc_feature 1..20: note = CBLB_2 gRNA
  source 1..20: mol_type = other RNA
cgctgggttgctccttcttc

SEQ ID NO: 46 - RNA, 20 nt, synthetic construct
  misc_feature 1..20: note = CBLB_3 gRNA
  source 1..20: mol_type = other RNA
cgtccaggcagacggcggtg

SEQ ID NO: 47 - RNA, 20 nt, synthetic construct
  misc_feature 1..20: note = DGKZ_1 gRNA
  source 1..20: mol_type = other RNA
gggacacgggcgggatcggt

SEQ ID NO: 48 - RNA, 20 nt, synthetic construct
  misc_feature 1..20: note = DGKZ_2 gRNA
  source 1..20: mol_type = other RNA
ggagcgagcgcgcgccatgg

SEQ ID NO: 49 - RNA, 20 nt, synthetic construct
  misc_feature 1..20: note = DGKZ_3 gRNA
  source 1..20: mol_type = other RNA
tcttcgggcacaggtgagcg

SEQ ID NO: 50 - RNA, 20 nt, synthetic construct
  misc_feature 1..20: note = MYB_1 gRNA
  source 1..20: mol_type = other RNA
gccgaatgggagcggcgacc

SEQ ID NO: 51 - RNA, 20 nt, synthetic construct
  misc_feature 1..20: note = MYB_2 gRNA
  source 1..20: mol_type = other RNA
ggatccctcgccgacacccg

SEQ ID NO: 52 - RNA, 20 nt, synthetic construct
  misc_feature 1..20: note = MYB_3 gRNA
  source 1..20: mol_type = other RNA
gaaacttcgccccagcggtg

SEQ ID NO: 53 - RNA, 20 nt, synthetic construct
  misc_feature 1..20: note = RASA2_1 gRNA
  source 1..20: mol_type = other RNA
gcacgggccgggcggcacca

SEQ ID NO: 54 - RNA, 20 nt, synthetic construct
  misc_feature 1..20: note = RASA2_2 gRNA
  source 1..20: mol_type = other RNA
tgctgcggcggcttcttccg

SEQ ID NO: 55 - RNA, 20 nt, synthetic construct
  misc_feature 1..20: note = RASA2_3 gRNA
  source 1..20: mol_type = other RNA
gcgctgggcgcgaggctgag

SEQ ID NO: 56 - RNA, 20 nt, synthetic construct
  misc_feature 1..20: note = ELOB_1 gRNA
  source 1..20: mol_type = other RNA
cgaactccttgggctagaag

SEQ ID NO: 57 - RNA, 20 nt, synthetic construct
  misc_feature 1..20: note = ELOB_2 gRNA
  source 1..20: mol_type = other RNA
tgcggccgccatcccgacgg

SEQ ID NO: 58 - RNA, 20 nt, synthetic construct
  misc_feature 1..20: note = ELOB_3 gRNA
  source 1..20: mol_type = other RNA
ctggaagcgggcggtatcga

SEQ ID NO: 59 - RNA, 20 nt, synthetic construct
  misc_feature 1..20: note = GATA3_1 gRNA
  source 1..20: mol_type = other RNA
cggagggtacctctgcaccg

SEQ ID NO: 60 - RNA, 20 nt, synthetic construct
  misc_feature 1..20: note = GATA3_2 gRNA
  source 1..20: mol_type = other RNA
tcgacgaggaggctccaccc

SEQ ID NO: 61 - RNA, 20 nt, synthetic construct
  misc_feature 1..20: note = GATA3_3 gRNA
  source 1..20: mol_type = other RNA
cagggctgactgttacgact

SEQ ID NO: 62 - RNA, 20 nt, synthetic construct
  misc_feature 1..20: note = CISH_1 gRNA
  source 1..20: mol_type = other RNA
tgtcctgcgcccgcgcgccc

SEQ ID NO: 63 - RNA, 20 nt, synthetic construct
  misc_feature 1..20: note = CISH_2 gRNA
  source 1..20: mol_type = other RNA
ggcggctggagggaaccagt

SEQ ID NO: 64 - RNA, 20 nt, synthetic construct
  misc_feature 1..20: note = CISH_3 gRNA
  source 1..20: mol_type = other RNA
gtggcgcggaccgcctgcga

SEQ ID NO: 65 - RNA, 20 nt, synthetic construct
  misc_feature 1..20: note = PRDM1_1 gRNA
  source 1..20: mol_type = other RNA
agaggcaagagcagcgaccg

SEQ ID NO: 66 - RNA, 20 nt, synthetic construct
  misc_feature 1..20: note = PRDM1_2 gRNA
  source 1..20: mol_type = other RNA
gacgcggggagaatgtggac

SEQ ID NO: 67 - RNA, 20 nt, synthetic construct
  misc_feature 1..20: note = PRDM1_3 gRNA
  source 1..20: mol_type = other RNA
ttgcctctccgcaacactgg

SEQ ID NO: 68 - RNA, 20 nt, synthetic construct
  misc_feature 1..20: note = non-targeting_sp_1 gRNA
  source 1..20: mol_type = other RNA
acggaggctaagcgtcgcaa

SEQ ID NO: 69 - RNA, 86 nt, synthetic construct
  misc_feature 1..86: note = SpCas9 gRNA scaffold sequence (RNA)
  source 1..86: mol_type = other RNA
gtttaagagctatgctggaaacagcatagcaagtttaaataaggctagtccgttatcaacttgaaaaagtggcaccgagtcggtgc

SEQ ID NO: 70 - protein, 65 aa, synthetic construct
  REGION 1..65: note = KRAB (AA)
RTLVTFKDVFVDFTREEWKLLDTAQQILYRNVMLENYKNLVSLGYQLTKPDVILRLEKGEEPWLV

SEQ ID NO: 71 - DNA, 195 bp, synthetic construct
  misc_feature 1..195: note = KRAB (nt)
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 72 - DNA, 4509 bp, synthetic construct
  misc_feature 1..4509: note = dSpCas9 KRAB- (nt)
  source 1..4509: mol_type = other DNA
ccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 73 - protein, 1503 aa, synthetic construct
  REGION 1..1503: note = dSpCas9- KRAB (AA)
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 74 - DNA, 6207 bp, synthetic construct
  misc_feature 1..6207: note = DNMT3A/L-dCas9-KRAB (nt)
  source 1..6207: mol_type = other DNA
cacgttaaccacgatcaggagtttgacccccctaaggtgtacccacccgtgccagccgagaagaggaagcccatccgcgtgctgtccctgttcgacggcatcgccacaggcctgctggtgctgaaggatctgggcatccaggtggacagatatatcgcctccgaggtgtgcgaggattctatcaccgtgggcatggtgaggcaccagggcaagatcatgtacgtgggcgacgtgcgcagcgtgacacagaagcacatccaggagtggggacccttcgacctggtcatcggaggcagcccctgtaatgacctgtccatcgtgaaccctgcaaggaagggcctgtatgagggaaccggcagactgttctttgagttctacaggctgctgcacgacgcccgccctaaggagggcgatgacaggccattcttttggctgtttgagaacgtggtggccatgggcgtgagcgacaagcgggatatctccagattcctggagtctaatcccgtgatgatcgatgcaaaggaggtgtctgccgcacacagggcaaggtacttttggggaaatctgcctggcatgaaccgcccactggccagcaccgtgaacgacaagctggagctgcaggagtgcctggagcacggaaggatcgccaagttctccaaggtgcggacaatcaccacaagatctaacagcatcaagcagggcaaggatcagcacttccccgtgttcatgaatgagaaggaggacatcctgtggtgtaccgagatggagcgcgtgttcggctttccagtgcactatacagacgtgagcaatatgagccggctggcaaggcagagactgctgggccggtcctggtctgtgccagtgatcagacacctgttcgcccccctgaaggagtactttgcctgcgtgtctagcggcaactctaatgccaacagcagaggcccttccttttcctctggcctggtgccactgtctctgaggggcagccacatgggccccatggagatctacaagaccgtgtccgcctggaagaggcagcctgtgcgcgtgctgtctctgttccgcaacatcgacaaggtgctgaagagcctgggctttctggagagcggatccggatctggaggaggcaccctgaagtatgtggaggatgtgacaaatgtggtgcggagagatgtggagaagtggggccccttcgatctggtgtacggatccacccagccactgggaagctcctgcgataggtgtccaggatggtatatgttccagtttcacagaatcctgcagtacgcactgccaaggcaggagagccagcgccctttcttttggatctttatggacaacctgctgctgacagaggatgaccaggagacaacaacccgcttcctgcagacagaggcagtgaccctgcaggatgtgaggggacgcgactatcagaatgccatgcgggtgtggtctaacatccctggcctgaagagcaagcacgcccccctgacccctaaggaggaggagtacctgcaggcccaggtgcggagcagatccaagctggatgcccctaaggtggacctgctggtgaagaattgtctgctgccactgcgggagtacttcaagtactttagtcagaatagcctgccactggaggcaagcggatccggaagggcatctcctggaatcccaggaagcacccgcaaccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggacgccatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 75 - protein, 2069 aa, synthetic construct
  REGION 1..2069: note = DNMT3A/L-dCas9-KRAB(AA)
HVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLEASGSGRASPGIPGSTRNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 76 - DNA, 4509 bp, synthetic construct
  misc_feature 1..4509: note = VP64-dSpCas9-NLS-VP64 (nt)
  source 1..4509: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaacccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 77 - protein, 1503 aa, synthetic construct
  REGION 1..1503: note = VP64-dSpCas9-NLS-VP64 (AA)
DALDDFDLDMLGSDALDDFDLDMLGSDALDDFDLDMLGSDALDDFDLDMVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASRADALDDFDLDMLGSDALDDFDLDMLGSDALDDFDLDMLGSDALDDFDLDML

SEQ ID NO: 78 - DNA, 20 bp, synthetic construct
  misc_feature 1..20: note = IL-2_1 target site
  source 1..20: mol_type = other DNA
gagagctatcacctaagtgt

SEQ ID NO: 79 - RNA, 20 nt, synthetic construct
  misc_feature 1..20: note = IL-2_1 gRNA
  source 1..20: mol_type = other RNA
gagagctatcacctaagtgt

SEQ ID NO: 80 - DNA, 20 bp, synthetic construct
  misc_feature 1..20: note = Med12_1 target site
  source 1..20: mol_type = other DNA
accattgccggaaactaccg

SEQ ID NO: 81 - DNA, 20 bp, synthetic construct
  misc_feature 1..20: note = Med12_2 target site
  source 1..20: mol_type = other DNA
gtgccccgggagtttttcgg

SEQ ID NO: 82 - DNA, 20 bp, synthetic construct
  misc_feature 1..20: note = Med12_3 target site
  source 1..20: mol_type = other DNA
acggcggccgagagacaaca

SEQ ID NO: 83 - DNA, 20 bp, synthetic construct
  misc_feature 1..20: note = Med12_4 target site
  source 1..20: mol_type = other DNA
cgaggtacgccgggaaccat

SEQ ID NO: 84 - DNA, 20 bp, synthetic construct
  misc_feature 1..20: note = Med12_5 target site
  source 1..20: mol_type = other DNA
cgccaccgccgaaaaactcc

SEQ ID NO: 85 - DNA, 20 bp, synthetic construct
  misc_feature 1..20: note = Med12_6 target site
  source 1..20: mol_type = other DNA
cgatggttcccggcgtacct

SEQ ID NO: 86 - DNA, 20 bp, synthetic construct
  misc_feature 1..20: note = Med12_7 target site
  source 1..20: mol_type = other DNA
cggcggccgagagacaacaa

SEQ ID NO: 87 - DNA, 20 bp, synthetic construct
  misc_feature 1..20: note = Med12_8 target site
  source 1..20: mol_type = other DNA
tcctgagggtaaacatcggg

SEQ ID NO: 88 - DNA, 20 bp, synthetic construct
  misc_feature 1..20: note = Med12_9 target site
  source 1..20: mol_type = other DNA
ttcgtagctcaagatcccga

SEQ ID NO: 89 - DNA, 20 bp, synthetic construct
  misc_feature 1..20: note = Med12_10 target site
  source 1..20: mol_type = other DNA
gctgactgggggaacgggaa

SEQ ID NO: 90 - DNA, 20 bp, synthetic construct
  misc_feature 1..20: note = Med12_11 target site
  source 1..20: mol_type = other DNA
ggctggtgcctccggcgcta

SEQ ID NO: 91 - RNA, 20 nt, synthetic construct
  misc_feature 1..20: note = Med12_1 gRNA
  source 1..20: mol_type = other RNA
accattgccggaaactaccg

SEQ ID NO: 92 - RNA, 20 nt, synthetic construct
  misc_feature 1..20: note = Med12_2 gRNA
  source 1..20: mol_type = other RNA
gtgccccgggagtttttcgg

SEQ ID NO: 93 - RNA, 20 nt, synthetic construct
  misc_feature 1..20: note = Med12_3 gRNA
  source 1..20: mol_type = other RNA
acggcggccgagagacaaca

SEQ ID NO: 94 - RNA, 20 nt, synthetic construct
  misc_feature 1..20: note = Med12_4 gRNA
  source 1..20: mol_type = other RNA
cgaggtacgccgggaaccat

SEQ ID NO: 95 - RNA, 20 nt, synthetic construct
  misc_feature 1..20: note = Med12_5 gRNA
  source 1..20: mol_type = other RNA
cgccaccgccgaaaaactcc

SEQ ID NO: 96 - RNA, 20 nt, synthetic construct
  misc_feature 1..20: note = Med12_6 gRNA
  source 1..20: mol_type = other RNA
cgatggttcccggcgtacct

SEQ ID NO: 97 - RNA, 20 nt, synthetic construct
  misc_feature 1..20: note = Med12_7 gRNA
  source 1..20: mol_type = other RNA
cggcggccgagagacaacaa

SEQ ID NO: 98 - RNA, 20 nt, synthetic construct
  misc_feature 1..20: note = Med12_8 gRNA
  source 1..20: mol_type = other RNA
tcctgagggtaaacatcggg

SEQ ID NO: 99 - RNA, 20 nt, synthetic construct
  misc_feature 1..20: note = Med12_9 gRNA
  source 1..20: mol_type = other RNA
ttcgtagctcaagatcccga

SEQ ID NO: 100 - RNA, 20 nt, synthetic construct
  misc_feature 1..20: note = Med12_10 gRNA
  source 1..20: mol_type = other RNA
gctgactgggggaacgggaa

SEQ ID NO: 101 - RNA, 20 nt, synthetic construct
  misc_feature 1..20: note = Med12_11 gRNA
  source 1..20: mol_type = other RNA
ggctggtgcctccggcgcta

SEQ ID NO: 102 - DNA, 20 bp, synthetic construct
  misc_feature 1..20: note = CCNC_1 target site
  source 1..20: mol_type = other DNA
aaagttccggcccgcggtag

SEQ ID NO: 103 - DNA, 20 bp, synthetic construct
  misc_feature 1..20: note = CCNC_2 target site
  source 1..20: mol_type = other DNA
gggccggaacttttgtcgat

SEQ ID NO: 104 - DNA, 20 bp, synthetic construct
  misc_feature 1..20: note = CCNC_3 target site
  source 1..20: mol_type = other DNA
cgacggcgaaaggaagagga

SEQ ID NO: 105 - DNA, 20 bp, synthetic construct
  misc_feature 1..20: note = CCNC_4 target site
  source 1..20: mol_type = other DNA
cgaggagcgcggttaccgga

SEQ ID NO: 106 - DNA, 20 bp, synthetic construct
  misc_feature 1..20: note = CCNC_5 target site
  source 1..20: mol_type = other DNA
cggccggcgtgaaggagact

SEQ ID NO: 107 - DNA, 20 bp, synthetic construct
  misc_feature 1..20: note = CCNC_6 target site
  source 1..20: mol_type = other DNA
tcacgagagctcgcggcggt

SEQ ID NO: 108 - DNA, 20 bp, synthetic construct
  misc_feature 1..20: note = CCNC_7 target site
  source 1..20: mol_type = other DNA
ctgggtctatggtcgctccg

SEQ ID NO: 109 - DNA, 20 bp, synthetic construct
  misc_feature 1..20: note = CCNC_8 target site
  source 1..20: mol_type = other DNA
gaacttttgtcgataggaac

SEQ ID NO: 110 - DNA, 20 bp, synthetic construct
  misc_feature 1..20: note = CCNC_9 target site
  source 1..20: mol_type = other DNA
gctgatttgatcgaggagcg

SEQ ID NO: 111 - DNA, 20 bp, synthetic construct
  misc_feature 1..20: note = CCNC_10 target site
  source 1..20: mol_type = other DNA
ggaggagcgcggttaccgga

SEQ ID NO: 112 - DNA, 20 bp, synthetic construct
  misc_feature 1..20: note = CCNC_11 target site
  source 1..20: mol_type = other DNA
gtgggtctatggtcgctccg

SEQ ID NO: 113 - RNA, 20 nt, synthetic construct
  misc_feature 1..20: note = CCNC_1 gRNA
  source 1..20: mol_type = other RNA
aaagttccggcccgcggtag

SEQ ID NO: 114 - RNA, 20 nt, synthetic construct
  misc_feature 1..20: note = CCNC_2 gRNA
  source 1..20: mol_type = other RNA
gggccggaacttttgtcgat

SEQ ID NO: 115 - RNA, 20 nt, synthetic construct
  misc_feature 1..20: note = CCNC_3 gRNA
  source 1..20: mol_type = other RNA
cgacggcgaaaggaagagga

SEQ ID NO: 116 - RNA, 20 nt, synthetic construct
  misc_feature 1..20: note = CCNC_4 gRNA
  source 1..20: mol_type = other RNA
cgaggagcgcggttaccgga

SEQ ID NO: 117 - RNA, 20 nt, synthetic construct
  misc_feature 1..20: note = CCNC_5 gRNA
  source 1..20: mol_type = other RNA
cggccggcgtgaaggagact

SEQ ID NO: 118 - RNA, 20 nt, synthetic construct
  misc_feature 1..20: note = CCNC_6 gRNA
  source 1..20: mol_type = other RNA
tcacgagagctcgcggcggt

SEQ ID NO: 119 - RNA, 20 nt, synthetic construct
  misc_feature 1..20: note = CCNC_7 gRNA
  source 1..20: mol_type = other RNA
ctgggtctatggtcgctccg

SEQ ID NO: 120 - RNA, 20 nt, synthetic construct
  misc_feature 1..20: note = CCNC_8 gRNA
  source 1..20: mol_type = other RNA
gaacttttgtcgataggaac

SEQ ID NO: 121 - RNA, 20 nt, synthetic construct
  misc_feature 1..20: note = CCNC_9 gRNA
  source 1..20: mol_type = other RNA
gctgatttgatcgaggagcg

SEQ ID NO: 122 - RNA, 20 nt, synthetic construct
  misc_feature 1..20: note = CCNC_10 gRNA
  source 1..20: mol_type = other RNA
ggaggagcgcggttaccgga

SEQ ID NO: 123 - RNA, 20 nt, synthetic construct
  misc_feature 1..20: note = CCNC_11 gRNA
  source 1..20: mol_type = other RNA
gtgggtctatggtcgctccg

SEQ ID NO: 124 - protein, 1053 aa, synthetic construct
  REGION 1..1053: note = SaCas9 (AA)
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 125 - protein, 1052 aa, synthetic construct
  REGION 1..1052: note = dSaCas9 (AA)
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 126 - protein, 1368 aa, synthetic construct [sequence omitted]

SEQ ID NO: 127 - protein, 1367 aa, synthetic construct
  REGION 1..1367: note = dSpCas9 (AA)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 128 - protein, 548 aa, synthetic construct
  REGION 1..548: note = ERF domain (AA)
MKTPADTGFAFPDWAYKPESSPGSRQIQLWHFILELLRKEEYQGVIAWQGDYGEFVIKDPDEVARLWGVRKCKPQMNYDKLSRALRYYYNKRILHKTKGKRFTYKFNFNKLVLVNYPFIDVGLAGGAVPQSAPPVPSGGSHFRFPPSTPSEVLSPTEDPRSPPACSSSSSSLFSAVVARRLGRGSVSDCSDGTSELEEPLGEDPRARPPGPPDLGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQPPPLGRRQRAAGEKAVAGADKSGGSAGGLAEGAGALAPPPPPPQIKVEPISEGESEEVEVTDISDEDEEDGEVFKTPRAPPAPPKPEPGEAPGASQCMPLKLRFKRRWSEDCRLEGGGGPAGGFEDEGEDKKVRGEGPGEAGGPLTPRRVSSDLQHATAQLSLEHRDS

SEQ ID NO: 129 - protein, 228 aa, synthetic construct
  REGION 1..228: note = MXI1 domain (AA)
MERVKMINVQRLLEAAEFLERRERECEHGYASSFPSMPSPRLQHSKPPRRLSRAQKHSSGSSNTSTANRSTHNELEKNRRAHLRLCLERLKVLIPLGPDCTRHTTLGLLNKAKAHIKKLEEAERKSQHQLENLEREQRFLKWRLEQLQGPQEMERIRMDSIGSTISSDRSDSEREEIEVDVESTEFSHGEVDNISTTSISDIDDHSSLPSIGSDEGYSSASVKLSFTS

SEQ ID NO: 130 - DNA, 966 bp, synthetic construct
  misc_feature 1..966: note = DNMT3A (nt)
  source 1..966: mol_type = other DNA
acctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtatccaacatgagccgcttggcgaggcagagactgctgggccggtcatggagcgtgccagtcatccgccacctcttcgctccgctgaaggagtattttgcgtgtgtg

SEQ ID NO: 131 - protein, 322 aa, synthetic construct
  REGION 1..322: note = DNMT3A (AA)
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 132 - DNA, 1158 bp, synthetic construct
  misc_feature 1..1158: note = DNMT3L - Human (nt)
  source 1..1158: mol_type = other DNA
atggcggccatcccagccctggacccagaggccgagcccagcatggacgtgattttggtgggatccagtgagctctcaagctccgtttcacccgggacaggcagagatcttattgcatatgaagtcaaggctaaccagcgaaatatagaagacatctgcatctgctgcggaagtctccaggttcacacacagcaccctctgtttgagggagggatctgcgccccatgtaaggacaagttcctggatgccctcttcctgtacgacgatgacgggtaccaatcctactgctccatctgctgctccggagaaacgctgctcatctgcggaaaccctgattgcacccgatgctactgcttcgagtgtgtggatagcctggtcggccccgggacctcggggaaggtgcacgccatgagcaactgggtgtgctacctgtgcctgccgtcctcccgaagcgggctgctgcagcgtcggaggaagtggcgcagccagctcaaggccttctacgaccgagagtcggagaatccccttgagatgttcgaaaccgtgcctgtgtggaggagacagccagtccgggtgctgtccctttttgaagacatcaagaaagagctgacgagtttgggctttttggaaagtggttctgacccgggacaactgaagcatgtggttgatgtcacagacacagtgaggaaggatgtggaggagtggggacccttcgatcttgtgtacggcgccacacctcccctgggccacacctgtgaccgtcctcccagctggtacctgttccagttccaccggctcctgcagtacgcacggcccaagccaggcagccccaggcccttcttctggatgttcgtggacaatctggtgctgaacaaggaagacctggacgtcgcatctcgcttcctggagatggagccagtcaccatcccagatgtccacggcggatccttgcagaatgctgtccgcgtgtggagcaacatcccagccataaggagcaggcactgggctctggtttcggaagaagaattgtccctgctggcccagaacaagcagagctcgaagctcgcggccaagtggcccaccaagctggtgaagaactgctttctccccctaagagaatatttcaagtatttttcaacagaactcacttcctcttta

SEQ ID NO: 133 - protein, 386 aa, synthetic construct
  REGION 1..386: note = DNMT3L - Human (AA)
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 134 - DNA, 1629 bp, synthetic construct
  misc_feature 1..1629: note = DNMT3A/L v1 (nt)
  source 1..1629: mol_type = other DNA
aaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctt

SEQ ID NO: 135 - protein, 543 aa, synthetic construct
  REGION 1..543: note = DNMT3A/L v1 (AA)
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 136 - DNA, 1629 bp, synthetic construct
  misc_feature 1..1629: note = DNMT3A/L v2 (nt)
  source 1..1629: mol_type = other DNA
aaccacgatcaggagtttgacccccctaaggtgtacccacccgtgccagccgagaagaggaagcccatccgcgtgctgtccctgttcgacggcatcgccacaggcctgctggtgctgaaggatctgggcatccaggtggacagatatatcgcctccgaggtgtgcgaggattctatcaccgtgggcatggtgaggcaccagggcaagatcatgtacgtgggcgacgtgcgcagcgtgacacagaagcacatccaggagtggggacccttcgacctggtcatcggaggcagcccctgtaatgacctgtccatcgtgaaccctgcaaggaagggcctgtatgagggaaccggcagactgttctttgagttctacaggctgctgcacgacgcccgccctaaggagggcgatgacaggccattcttttggctgtttgagaacgtggtggccatgggcgtgagcgacaagcgggatatctccagattcctggagtctaatcccgtgatgatcgatgcaaaggaggtgtctgccgcacacagggcaaggtacttttggggaaatctgcctggcatgaaccgcccactggccagcaccgtgaacgacaagctggagctgcaggagtgcctggagcacggaaggatcgccaagttctccaaggtgcggacaatcaccacaagatctaacagcatcaagcagggcaaggatcagcacttccccgtgttcatgaatgagaaggaggacatcctgtggtgtaccgagatggagcgcgtgttcggctttccagtgcactatacagacgtgagcaatatgagccggctggcaaggcagagactgctgggccggtcctggtctgtgccagtgatcagacacctgttcgcccccctgaaggagtactttgcctgcgtgtctagcggcaactctaatgccaacagcagaggcccttccttttcctctggcctggtgccactgtctctgaggggcagccacatgggccccatggagatctacaagaccgtgtccgcctggaagaggcagcctgtgcgcgtgctgtctctgttccgcaacatcgacaaggtgctgaagagcctgggctttctggagagcggatccggatctggaggaggcaccctgaagtatgtggaggatgtgacaaatgtggtgcggagagatgtggagaagtggggccccttcgatctggtgtacggatccacccagccactgggaagctcctgcgataggtgtccaggatggtatatgttccagtttcacagaatcctgcagtacgcactgccaaggcaggagagccagcgccctttcttttggatctttatggacaacctgctgctgacagaggatgaccaggagacaacaacccgcttcctgcagacagaggcagtgaccctgcaggatgtgaggggacgcgactatcagaatgccatgcgggtgtggtctaacatccctggcctgaagagcaagcacgcccccctgacccctaaggaggaggagtacctgcaggcccaggtgcggagcagatccaagctggatgcccctaaggtggacctgctggtgaagaattgtctgctgccactgcgggagtacttcaagtactttagtcagaatagcctgccactg

SEQ ID NO: 137 - protein, 543 aa, synthetic construct; identical to SEQ ID NO: 135
  REGION 1..543: note = DNMT3A/L v2 (AA)

SEQ ID NO: 138 - protein, 1503 aa, synthetic construct; identical to SEQ ID NO: 73
  REGION 1..1503: note = dSpCas9-KRAB (AA)

SEQ ID NO: 139 - protein, 2067 aa, synthetic construct
  REGION 1..2067: note = dSpCas9-KRAB-DNTM3A/L (AA)
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLEASGSGRASPGIPGSTRNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 140 - protein, 2132 aa, synthetic construct
  REGION 1..2132: note = DNMT3A/L-XTEN80-dSpCas9-KRAB (AA)
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 141 - protein, 2132 aa, synthetic construct
  REGION 1..2132: note = DNMT3A/L(CRISPROFF)-XTEN80-dSpCas9-KRAB (AA)
SNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 142 - protein, 49 aa, synthetic construct
  REGION 1..49: note = VP64 (AA)
DALDDFDLDMLGSDALDDFDLDMLGSDALDDFDLDMLGSDALDDFDLDM

SEQ ID NO: 143 - protein, 1503 aa, synthetic construct; identical to SEQ ID NO: 77
  REGION 1..1503: note = VP64-dSpCas9-NLS-VP64 (AA)

SEQ ID NO: 144 - DNA, 20 bp, synthetic construct
  misc_feature 1..20: note = CD28_1 target site
  source 1..20: mol_type = other DNA
ctgactgcagcatttcacac

SEQ ID NO: 145 - DNA, 20 bp, synthetic construct
  misc_feature 1..20: note = CD28_2 target site
  source 1..20: mol_type = other DNA
gctgcagtcaggatgccttg

SEQ ID NO: 146 - DNA, 20 bp, synthetic construct
  misc_feature 1..20: note = CD28_3 target site
  source 1..20: mol_type = other DNA
ccttgatcatgtgccctaag

SEQ ID NO: 147 - DNA, 20 bp, synthetic construct
  misc_feature 1..20: note = EOMES_1 target site
  source 1..20: mol_type = other DNA
cgcagtagcggcccgcgagt

SEQ ID NO: 148 - DNA, 20 bp, synthetic construct
  misc_feature 1..20: note = EOMES_2 target site
  source 1..20: mol_type = other DNA
cgggccgctactgcgcgtac

SEQ ID NO: 149 - DNA, 20 bp, synthetic construct
  misc_feature 1..20: note = EOMES_3 target site
  source 1..20: mol_type = other DNA
gctacccacctgccgactcg

SEQ ID NO: 150 - DNA, 20 bp, synthetic construct
  misc_feature 1..20: note = LCP2_1 target site
  source 1..20: mol_type = other DNA
tccagtcaccccaacccagt

SEQ ID NO: 151 - DNA, 20 bp, synthetic construct
  misc_feature 1..20: note = LCP2_2 target site
  source 1..20: mol_type = other DNA
ggtgctacactgtgcagaca

SEQ ID NO: 152 - DNA, 20 bp, synthetic construct
  misc_feature 1..20: note = LCP2_3 target site
  source 1..20: mol_type = other DNA
cagggttggtaattccccac

SEQ ID NO: 153 - DNA, 20 bp, synthetic construct
  misc_feature 1..20: note = TBX21_1 target site
  source 1..20: mol_type = other DNA
cagggccgaggtggcggagt

SEQ ID NO: 154 - DNA, 20 bp, synthetic construct
  misc_feature 1..20: note = TBX21_2 target site
  source 1..20: mol_type = other DNA
ctcgcttctctccaccatgg

SEQ ID NO: 155 - DNA, 20 bp, synthetic construct
  misc_feature 1..20: note = TBX21_3 target site
  source 1..20: mol_type = other DNA
ctaggcaaattctacgctct

SEQ ID NO: 156 - DNA, 20 bp, synthetic construct
  misc_feature 1..20: note = VAV1_4 target site
  source 1..20: mol_type = other DNA
cctgtaactgtcgccctgag

SEQ ID NO: 157 - RNA, 20 nt, synthetic construct
  misc_feature 1..20: note = CD28_1 gRNA
  source 1..20: mol_type = other RNA
ctgactgcagcatttcacac

SEQ ID NO: 158 - RNA, 20 nt, synthetic construct
  misc_feature 1..20: note = CD28_2 gRNA
  source 1..20: mol_type = other RNA
gctgcagtcaggatgccttg

SEQ ID NO: 159 - RNA, 20 nt, synthetic construct
  misc_feature 1..20: note = CD28_3 gRNA
  source 1..20: mol_type = other RNA
ccttgatcatgtgccctaag

SEQ ID NO: 160 - RNA, 20 nt, synthetic construct
  misc_feature 1..20: note = EOMES_1 gRNA
  source 1..20: mol_type = other RNA
cgcagtagcggcccgcgagt

SEQ ID NO: 161 - RNA, 20 nt, synthetic construct
  misc_feature 1..20: note = EOMES_2 gRNA
  source 1..20: mol_type = other RNA
cgggccgctactgcgcgtac

SEQ ID NO: 162 - RNA, 20 nt, synthetic construct
  misc_feature 1..20: note = EOMES_3 gRNA
  source 1..20: mol_type = other RNA
gctacccacctgccgactcg

SEQ ID NO: 163 - RNA, 20 nt, synthetic construct
  misc_feature 1..20: note = LCP2_1 gRNA
  source 1..20: mol_type = other RNA
tccagtcaccccaacccagt

SEQ ID NO: 164 - RNA, 20 nt, synthetic construct
  misc_feature 1..20: note = LCP2_2 gRNA
  source 1..20: mol_type = other RNA
ggtgctacactgtgcagaca

SEQ ID NO: 165 - RNA, 20 nt, synthetic construct
  misc_feature 1..20: note = LCP2_3 gRNA
  source 1..20: mol_type = other RNA
cagggttggtaattccccac

SEQ ID NO: 166 - RNA, 20 nt, synthetic construct
  misc_feature 1..20: note = TBX21_1 gRNA
  source 1..20: mol_type = other RNA
cagggccgaggtggcggagt

SEQ ID NO: 167 - RNA, 20 nt, synthetic construct
  misc_feature 1..20: note = TBX21_2 gRNA
  source 1..20: mol_type = other RNA
ctcgcttctctccaccatgg

SEQ ID NO: 168 - RNA, 20 nt, synthetic construct
  misc_feature 1..20: note = TBX21_3 gRNA
  source 1..20: mol_type = other RNA
ctaggcaaattctacgctct

SEQ ID NO: 169 - RNA, 20 nt, synthetic construct
  misc_feature 1..20: note = VAV1_4 gRNA
  source 1..20: mol_type = other RNA
cctgtaactgtcgccctgag

SEQ ID NO: 170 - DNA, 20 bp, synthetic construct
  misc_feature 1..20: note = VAV1_5 target site
  source 1..20: mol_type = other DNA
ccaggcctgtgtcgagtggg

SEQ ID NO: 171 - RNA, 20 nt, synthetic construct
  misc_feature 1..20: note = VAV1_5 gRNA
  source 1..20: mol_type = other RNA
ccaggcctgtgtcgagtggg

SEQ ID NO: 172 - DNA, 20 bp, synthetic construct
  misc_feature 1..20: note = BATF_1 target site
  source 1..20: mol_type = other DNA
actcacgctggaagtcacat

SEQ ID NO: 173 - DNA, 20 bp, synthetic construct
  misc_feature 1..20: note = BATF_2 target site
  source 1..20: mol_type = other DNA
aagtccgtcttctgtcaaca

SEQ ID NO: 174 - DNA, 20 bp, synthetic construct
  misc_feature 1..20: note = BATF_3 target site
  source 1..20: mol_type = other DNA
gcagagggactgctccccaa

SEQ ID NO: 175 - DNA, 20 bp, synthetic construct
  misc_feature 1..20: note = IRF4_1 target site
  source 1..20: mol_type = other DNA
ccgcttcggggactgtcact

SEQ ID NO: 176 - DNA, 20 bp, synthetic construct
  misc_feature 1..20: note = IRF4_2 target site
  source 1..20: mol_type = other DNA
actttgcaagccgagagccg

SEQ ID NO: 177 - DNA, 20 bp, synthetic construct
  misc_feature 1..20: note = IRF4_3 target site
  source 1..20: mol_type = other DNA
gtccaacccccggcccccac

SEQ ID NO: 178 - RNA, 20 nt, synthetic construct
  misc_feature 1..20: note = BATF_1 gRNA spacer
  source 1..20: mol_type = other RNA
actcacgctggaagtcacat

SEQ ID NO: 179 - RNA, 20 nt, synthetic construct
  misc_feature 1..20: note = BATF_2 gRNA spacer
  source 1..20: mol_type = other RNA
aagtccgtcttctgtcaaca

SEQ ID NO: 180 - RNA, 20 nt, synthetic construct
  misc_feature 1..20: note = BATF_3 gRNA spacer
  source 1..20: mol_type = other RNA
gcagagggactgctccccaa

SEQ ID NO: 181 - RNA, 20 nt, synthetic construct
  misc_feature 1..20: note = IRF4_1 gRNA spacer
  source 1..20: mol_type = other RNA
ccgcttcggggactgtcact

SEQ ID NO: 182 - RNA, 20 nt, synthetic construct
  misc_feature 1..20: note = IRF4_2 gRNA spacer
  source 1..20: mol_type = other RNA
actttgcaagccgagagccg

SEQ ID NO: 183 - RNA, 20 nt, synthetic construct
  misc_feature 1..20: note = IRF4_3 gRNA spacer
  source 1..20: mol_type = other RNA
gtccaacccccggcccccac

SEQ ID NO: 184 - DNA, 20 bp, synthetic construct
  misc_feature 1..20: note = LAT_1 target site
  source 1..20: mol_type = other DNA
ggctgggacgcaggggtaac

SEQ ID NO: 185 - DNA, 20 bp, synthetic construct
  misc_feature 1..20: note = LAT_2 target site
  source 1..20: mol_type = other DNA
ccaccccaggcactcaccaa

SEQ ID NO: 186 - DNA, 20 bp, synthetic construct
  misc_feature 1..20: note = LAT_3 target site
  source 1..20: mol_type = other DNA
ctgtggtgagcgccgggcga

SEQ ID NO: 187 - DNA, 20 bp, synthetic construct
  misc_feature 1..20: note = LCP2_4 target site
  source 1..20: mol_type = other DNA
aaagacatcggctccaacag

SEQ ID NO: 188 - DNA, 20 bp, synthetic construct
  misc_feature 1..20: note = LCP2_5 target site
  source 1..20: mol_type = other DNA
agccgttgctttctgggatc

SEQ ID NO: 189 - DNA, 20 bp, synthetic construct
  misc_feature 1..20: note = CD28_4 target site
  source 1..20: mol_type = other DNA
tcgtcaggacaaagatgctc

SEQ ID NO: 190 - DNA, 20 bp, synthetic construct
  misc_feature 1..20: note = CD28_5 target site
  source 1..20: mol_type = other DNA
cgtggatgacggagactctc

SEQ ID NO: 191 - DNA, 20 bp, synthetic construct
  misc_feature 1..20: note = CD28_6 target site
  source 1..20: mol_type = other DNA
ctgagagtctccgtcatcca

SEQ ID NO: 192 - RNA, 20 nt, synthetic construct
  misc_feature 1..20: note = LAT_1 gRNA spacer
  source 1..20: mol_type = other RNA
ggctgggacgcaggggtaac

SEQ ID NO: 193 - RNA, 20 nt, synthetic construct
  misc_feature 1..20: note = LAT_2 gRNA spacer
  source 1..20: mol_type = other RNA
ccaccccaggcactcaccaa

SEQ ID NO: 194 - RNA, 20 nt, synthetic construct
  misc_feature 1..20: note = LAT_3 gRNA spacer
  source 1..20: mol_type = other RNA
ctgtggtgagcgccgggcga

SEQ ID NO: 195 - RNA, 20 nt, synthetic construct
  misc_feature 1..20: note = LCP2_4 gRNA spacer
  source 1..20: mol_type = other RNA
aaagacatcggctccaacag

SEQ ID NO: 196 - RNA, 20 nt, synthetic construct
  misc_feature 1..20: note = LCP2_5 gRNA spacer
  source 1..20: mol_type = other RNA
agccgttgctttctgggatc

SEQ ID NO: 197 - RNA, 20 nt, synthetic construct
  misc_feature 1..20: note = CD28_4 gRNA spacer
  source 1..20: mol_type = other RNA
tcgtcaggacaaagatgctc

SEQ ID NO: 198 - RNA, 20 nt, synthetic construct
  misc_feature 1..20: note = CD28_5 gRNA spacer
  source 1..20: mol_type = other RNA
cgtggatgacggagactctc

SEQ ID NO: 199 - RNA, 20 nt, synthetic construct
  misc_feature 1..20: note = CD28_6 gRNA spacer
  source 1..20: mol_type = other RNA
ctgagagtctccgtcatcca

SEQ ID NO: 200 - DNA, 20 bp, synthetic construct
  misc_feature 1..20: note = FAS_1 target site
  source 1..20: mol_type = other DNA
gacccgctcagtacggagtt

SEQ ID NO: 201 - DNA, 20 bp, synthetic construct
  misc_feature 1..20: note = FAS_2 target site
  source 1..20: mol_type = other DNA
tccccaactccgtactgagc

SEQ ID NO: 202 - DNA, 20 bp, synthetic construct
  misc_feature 1..20: note = FAS_3 target site
  source 1..20: mol_type = other DNA
gttggtggacccgctcagta

SEQ ID NO: 203 - DNA, 20 bp, synthetic construct
  misc_feature 1..20: note = FAS_4 target site
  source 1..20: mol_type = other DNA
ggacccgctcagtacggagt

SEQ ID NO: 204 - DNA, 20 bp, synthetic construct
  misc_feature 1..20: note = FAS_5 target site
  source 1..20: mol_type = other DNA
acccgctcagtacggagttg

SEQ ID NO: 205 - DNA, 20 bp, synthetic construct
  misc_feature 1..20: note = FAS_6 target site
  source 1..20: mol_type = other DNA
gaagcagtggttaagccgga

SEQ ID NO: 206 - DNA, 20 bp, synthetic construct
  misc_feature 1..20: note = Fli1_1 target site
  source 1..20: mol_type = other DNA
cgcgcggcggcccaggaggg

SEQ ID NO: 207 - DNA, 20 bp, synthetic construct
  misc_feature 1..20: note = Fli1_2 target site
  source 1..20: mol_type = other DNA
gcgctcgcagggggcacgca

SEQ ID NO: 208 - DNA, 20 bp, synthetic construct
  misc_feature 1..20: note = Fli1_3 target site
  source 1..20: mol_type = other DNA
acaacaacaaacgtgcacag

SEQ ID NO: 209 - DNA, 20 bp, synthetic construct
  misc_feature 1..20: note = Fli1_4 target site
  source 1..20: mol_type = other DNA
gagggcagggcgctcgcagg

SEQ ID NO: 210 - DNA, 20 bp, synthetic construct
  misc_feature 1..20: note = Fli1_5 target site
  source 1..20: mol_type = other DNA
aaacgtgcacaggggagtga

SEQ ID NO: 211 - DNA, 20 bp, synthetic construct
  misc_feature 1..20: note = Fli1_6 target site
  source 1..20: mol_type = other DNA
gagcgaaagagacagttaac

SEQ ID NO: 212 - RNA, 20 nt, synthetic construct
  misc_feature 1..20: note = FAS_1 gRNA spacer
  source 1..20: mol_type = other RNA
gacccgctcagtacggagtt

SEQ ID NO: 213 - RNA, 20 nt, synthetic construct
  misc_feature 1..20: note = FAS_2 gRNA spacer
  source 1..20: mol_type = other RNA
tccccaactccgtactgagc

SEQ ID NO: 214 - RNA, 20 nt, synthetic construct
  misc_feature 1..20: note = FAS_3 gRNA spacer
  source 1..20: mol_type = other RNA
gttggtggacccgctcagta

SEQ ID NO: 215 - RNA, 20 nt, synthetic construct
  misc_feature 1..20: note = FAS_4 gRNA spacer
  source 1..20: mol_type = other RNA
ggacccgctcagtacggagt

SEQ ID NO: 216 - RNA, 20 nt, synthetic construct
  misc_feature 1..20: note = FAS_5 gRNA spacer
  source 1..20: mol_type = other RNA
acccgctcagtacggagttg

SEQ ID NO: 217 - RNA, 20 nt, synthetic construct
  misc_feature 1..20: note = FAS_6 gRNA spacer
  source 1..20: mol_type = other RNA
gaagcagtggttaagccgga

SEQ ID NO: 218 - RNA, 20 nt, synthetic construct
  misc_feature 1..20: note = Fli1_1 gRNA spacer
  source 1..20: mol_type = other RNA
cgcgcggcggcccaggaggg

SEQ ID NO: 219 - RNA, 20 nt, synthetic construct
  misc_feature 1..20: note = Fli1_2 gRNA spacer
  source 1..20: mol_type = other RNA
gcgctcgcagggggcacgca

SEQ ID NO: 220 - RNA, 20 nt, synthetic construct
  misc_feature 1..20: note = Fli1_3 gRNA spacer
  source 1..20: mol_type = other RNA
acaacaacaaacgtgcacag

SEQ ID NO: 221 - RNA, 20 nt, synthetic construct
  misc_feature 1..20: note = Fli1_4 gRNA spacer
  source 1..20: mol_type = other RNA
gagggcagggcgctcgcagg

SEQ ID NO: 222 - RNA, 20 nt, synthetic construct
  misc_feature 1..20: note = Fli1_5 gRNA spacer
  source 1..20: mol_type = other RNA
aaacgtgcacaggggagtga

SEQ ID NO: 223 - RNA, 20 nt, synthetic construct
  misc_feature 1..20: note = Fli1_6 gRNA spacer
  source 1..20: mol_type = other RNA
gagcgaaagagacagttaac

SEQ ID NO: 224 - skipped

SEQ ID NO: 225 - protein, 6 aa, synthetic construct
  REGION 1..6: note = PAM - SaCas9
NNGRRT

SEQ ID NO: 226 - protein, 8 aa, synthetic construct
  REGION 1..8: note = PAM
NNNNGATT

SEQ ID NO: 227 - protein, 8 aa, synthetic construct
  REGION 1..8: note = PAM
NNNNRYAC

SEQ ID NO: 228 - protein, 7 aa, synthetic construct
  REGION 1..7: note = PAM
NNAGAAW

SEQ ID NO: 229 - protein, 6 aa, synthetic construct
  REGION 1..6: note = PAM
NAAAAC

SEQ ID NO: 230 - protein, 4 aa, synthetic construct
  REGION 1..4: note = PAM
TTTV

SEQ ID NO: 231 - protein, 4 aa, synthetic construct
  REGION 1..4: note = PAM
NGAN

SEQ ID NO: 232 - protein, 4 aa, synthetic construct
  REGION 1..4: note = PAM
NGNG

SEQ ID NO: 233 - protein, 4 aa, synthetic construct
  REGION 1..4: note = PAM
NGAG

SEQ ID NO: 234 - protein, 4 aa, synthetic construct
  REGION 1..4: note = PAM
NGCG

SEQ ID NO: 235 - protein, 87 aa, synthetic construct
  REGION 1..87: note = KRAB (AA)
RTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 236 - protein, 144 aa, synthetic construct
  REGION 1..144: note = SID4X domain (AA)
ASPKKKRKVEASGSGMNIQMLLEAADYLERREREAEHGYASMLPGSGMNIQMLLEAADYLERREREAEHGYASMLPGSGMNIQMLLEAADYLERREREAEHGYASMLPGSGMNIQMLLEAADYLERREREAEHGYASMLPSRSR

SEQ ID NO: 237 - protein, 60 aa, synthetic construct
  REGION 1..60: note = MAD-SID domain (AA)
MAAAVRMNIQMLLEAADYLERREREAEHGYASMLPYNNKDRDALKRRNKSKKNNSSSRST

SEQ ID NO: 238 - protein, 60 aa, synthetic construct
  REGION 1..60: note = DNMT3A - Human (AA)
HNEMEKNRRAHLRLCLEKLKGLVPLGPESSRHTTLSLLTKAKLHIKKLEDCDRKAVHQID

SEQ ID NO: 239 - protein, 60 aa, synthetic construct
  REGION 1..60: note = DNMT3B (AA)
QLQREQRHLKRQLEKLGIERIRMDSIGSTVSSERSDSDREEIDVDVESTDYLTGDLDWSS

SEQ ID NO: 240 - protein, 40 aa, synthetic construct
  REGION 1..40: note = DNMT3L - Murine (AA)
SSVSDSDERGSMQSLGSDEGYSSTSIKRIKLQDSHKACLG

SEQ ID NO: 241 - protein, 301 aa, synthetic construct
  REGION 1..301: note = DNMT3L - Murine (AA)
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 242 - protein, 853 aa, synthetic construct
  REGION 1..853: note = C-terminal human DNMT3L (AA)
MKGDTRHLNGEEDAGGREDSILVNGACSDQSSDSPPILEAIRTPEIRGRRSSSRLSKREVSSLLSYTQDLTGDGDGEDGDGSDTPVMPKLFRETRTRSESPAVRTRNNNSVSSRERHRPSPRSTRGRQGRNHVDESPVEFPATRSLRRRATASAGTPWPSPPSSYLTIDLTDDTEDTHGTPQSSSTPYARLAQDSQQGGMESPQVEADSGDGDSSEYQDGKEFGIGDLVWGKIKGFSWWPAMVVSWKATSKRQAMSGMRWVQWFGDGKFSEVSADKLVALGLFSQHFNLATFNKLVSYRKAMYHALEKARVRAGKTFPSSPGDSLEDQLKPMLEWAHGGFKPTGIEGLKPNNTQPVVNKSKVRRAGSRKLESRKYENKTRRRTADDSATSDYCPAPKRLKTNCYNNGKDRGDEDQSREQMASDVANNKSSLEDGCLSCGRKNPVSFHPLFEGGLCQTCRDRFLELFYMYDDDGYQSYCTVCCEGRELLLCSNTSCCRCFCVECLEVLVGTGTAAEAKLQEPWSCYMCLPQRCHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 243 - protein, 421 aa, synthetic construct
  REGION 1..421: note = Linker (AA)
MGSRETPSSCSKTLETLDLETSDSSSPDADSPLEEQWLKSSPALKEDSVDVVLEDCKEPLSPSSPPTGREMIRYEVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 244 - protein, 215 aa, synthetic construct
  REGION 1..215: note = LSD1 (AA)
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 245 - protein, 214 aa, synthetic construct
  REGION 1..214: note = EZH2 (AA)
MNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 246 - protein, 27 aa, synthetic construct
  REGION 1..27: note = SunTag GCN4 peptide (AA)
SSGNSNANSRGPSFSSGLVPLSLRGSH

SEQ ID NO: 247 - protein, 852 aa, synthetic construct
  REGION 1..852: note = GGSGG linker (AA)
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 248 - protein, 746 aa, synthetic construct
  REGION 1..746: note = p65AD (aa)
MGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 249 - protein, 22 aa, synthetic construct
  REGION 1..22: note = Rta domain (aa)
LLPKNYHLENEVARLKKLVGER

SEQ ID NO: 250 - protein, 5 aa, synthetic construct
  REGION 1..5: note = CREB binding protein (CBP) (AA)
GGSGG

SEQ ID NO: 251 - protein, 124 aa, synthetic construct
  REGION 1..124: note = p300 (aa)
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 252 - protein, 190 aa, synthetic construct
  REGION 1..190: note = p300 core (aa)
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 253 - protein, 2404 aa, synthetic construct
  REGION 1..2404: note = HSF1 domain (aa)
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQAILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 254 - protein, 2414 aa, synthetic construct
  REGION 1..2414: note = HSF1 (AA)
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 255 - protein, 617 aa, synthetic construct
  REGION 1..617: note = VPR (aa)
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 256 - protein, 124 aa, synthetic construct
  REGION 1..124: note = VPH (aa)
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 257 - protein, 489 aa, synthetic construct
  REGION 1..489: note = TET1 (AA)
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSAGALHSAAAVPAGPRLRGHREQRPAGAV

SEQ ID NO: 258 - protein, 522 aa, synthetic construct
  REGION 1..522: note = TET1 catalytic domain (aa)
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 259 - protein, 378 aa, synthetic construct
  REGION 1..378: note = Linker (AA)
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 260 - protein, 2136 aa, synthetic construct
  REGION 1..2136: note = Linker (AA)
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 261 - protein, 718 aa, synthetic construct
  REGION 1..718: note = Linker (AA)
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 262 - protein, 5 aa, synthetic construct
  REGION 1..5: note = Linker (AA)
GGGGS

SEQ ID NO: 263 - protein, 5 aa, synthetic construct
  REGION 1..5: note = Linker (AA)
GGGGG

SEQ ID NO: 264 - protein, 5 aa, synthetic construct
  REGION 1..5: note = Linker (AA)
GGAGG

SEQ ID NO: 265 - protein, 7 aa, synthetic construct
  REGION 1..7: note = Linker (AA)
GGGGSSS

SEQ ID NO: 266 - protein, 7 aa, synthetic construct
  REGION 1..7: note = Linker (AA)
GGGGAAA

SEQ ID NO: 267 - protein, 17 aa, synthetic construct
  REGION 1..17: note = XTEN80 (aa)
EASGSGRASPGIPGSTR

SEQ ID NO: 268 - protein, 8 aa, synthetic construct
  REGION 1..8: note = Linker (AA)
GIHGVPAA

SEQ ID NO: 269 - protein, 27 aa, synthetic construct
  REGION 1..27: note = NLS
KRPAATKKAGQAKKKKASDAKSLTAWS

SEQ ID NO: 270 - protein, 80 aa, synthetic construct
  REGION 1..80: note = NLS
GGPSSGAPPPSGGSPAGSPTSTEEGTSESATPESGPGTSTEPSEGSAPGSPAGSPTSTEEGTSTEPSEGSAPGTSTEPSE

SEQ ID NO: 271 - protein, 16 aa, synthetic construct
  REGION 1..16: note = NLS
SGSETPGTSESATPES

SEQ ID NO: 272 - protein, 7 aa, synthetic construct
  REGION 1..7: note = NLS
PKKKRKV

SEQ ID NO: 273 - protein, 16 aa, synthetic construct
  REGION 1..16: note = NLS
KRPAATKKAGQAKKKK

SEQ ID NO: 274 - protein, 9 aa, synthetic construct
  REGION 1..9: note = NLS
PAAKRVKLD

SEQ ID NO: 275 - protein, 11 aa, synthetic construct
  REGION 1..11: note = NLS
RQRRNELKRSP

SEQ ID NO: 276 - protein, 38 aa, synthetic construct
  REGION 1..38: note = NLS
NQSSNFGPMKGGNFGGRSSGPYGGGGQYFAKPRNQGGY

SEQ ID NO: 277 - protein, 42 aa, synthetic construct
  REGION 1..42: note = NLS
RMRIZFKNKGKDTAELRRRRVEVSVELRKAKKDEQILKRRNV

SEQ ID NO: 278 - protein, 8 aa, synthetic construct
  REGION 1..8: note = NLS
VSRKRPRP

SEQ ID NO: 279 - protein, 8 aa, synthetic construct
  REGION 1..8: note = NLS
PPKKARED

SEQ ID NO: 280 - protein, 8 aa, synthetic construct
  REGION 1..8: note = NLS
PQPKKKPL

SEQ ID NO: 281 - protein, 12 aa, synthetic construct
  REGION 1..12: note = NLS
SALIKKKKKMAP

SEQ ID NO: 282 - protein, 5 aa, synthetic construct
  REGION 1..5: note = NLS
DRLRR

SEQ ID NO: 283 - protein, 7 aa, synthetic construct
  REGION 1..7: note = NLS
PKQKKRK

SEQ ID NO: 284 - protein, 10 aa, synthetic construct
  REGION 1..10: note = NLS
RKLKKKIKKL

SEQ ID NO: 285 - protein, 10 aa, synthetic construct
  REGION 1..10: note = Linker (AA)
REKKKFLKRR

SEQ ID NO: 286 - protein, 20 aa, synthetic construct
  REGION 1..20: note = FLAG tag (AA)
KRKGDEVDGVDEVAKKKSKK

SEQ ID NO: 287 - protein, 17 aa, synthetic construct
  REGION 1..17: note = 3x FLAG tag (AA)
RKCLQAGMNLEARKTKK

SEQ ID NO: 288 - protein, 15 aa, synthetic construct
  REGION 1..15: note = HA tag (AA)
PKKKRKVGIHGVPAA

SEQ ID NO: 289 - protein, 8 aa, synthetic construct
  REGION 1..8: note = His tag (AA)
DYKDDDDK

SEQ ID NO: 290 - protein, 23 aa, synthetic construct
  REGION 1..23: note = N term Npu Intein (aa)
MDYKDHDGDYKDHDIDYKDDDDK

SEQ ID NO: 291 - protein, 9 aa, synthetic construct
  REGION 1..9: note = C term Npu Intein (aa)
YPYDVPDYA

SEQ ID NO: 292 - protein, 6 aa, synthetic construct
  REGION 1..6: note = FAS_7 target site
HHHHHH

SEQ ID NO: 293 - protein, 85 aa, synthetic construct
  REGION 1..85: note = FAS_8 target site
CLSYETEILTVEYGLLPIGKIVEKRIECTVYSVDNNGNIYTQPVAQWHDRGEQEVFEYCLEDGSLIRATKDHKFMTVDGQMLPID

SEQ ID NO: 294 - protein, 35 aa, synthetic construct
  REGION 1..35: note = FAS_9 target site
IKIATRKYLGKQNVYDIGVERDHNFALKNGFIASN

SEQ ID NO: 295 - DNA, 20 bp, synthetic construct
  misc_feature 1..20: note = FAS_10 target site
  source 1..20: mol_type = other DNA
ttccccaactccgtactgag

SEQ ID NO: 296 - RNA, 20 nt, synthetic construct
  modified_base 8: mod_base = OTHER; note = thymine
  modified_base 10..12: mod_base = OTHER; note = thymine
  modified_base 16..17: mod_base = OTHER; note = thymine
  misc_feature 1..20: note = FAS_7 gRNA spacer
  source 1..20: mol_type = other RNA
gggaagctctttcacttcgg

SEQ ID NO: 297 - RNA, 23 nt, synthetic construct
  modified_base 3: mod_base = OTHER; note = thymine
  modified_base 5: mod_base = OTHER; note = thymine
  modified_base 9: mod_base = OTHER; note = thymine
  modified_base 13: mod_base = OTHER; note = thymine
  modified_base 17: mod_base = OTHER; note = thymine
  modified_base 21: mod_base = OTHER; note = thymine
  misc_feature 1..23: note = FAS_8 gRNA spacer
  source 1..23: mol_type = other RNA
actgtaagtcgctgcctgagtgg

SEQ ID NO: 298 - RNA, 20 nt, synthetic construct
  modified_base 8: mod_base = OTHER; note = thymine
  misc_feature 1..20: note = FAS_9 gRNA spacer
  source 1..20: mol_type = other RNA
gagcgggtccaccaacccgc

SEQ ID NO: 299 - RNA, 20 nt, synthetic construct
  misc_feature 1..20: note = FAS_10 gRNA spacer
  source 1..20: mol_type = other RNA
ttccccaactccgtactgag

SEQ ID NO: 300 - RNA, 20 nt, synthetic construct
  misc_feature 1..20: note = TGFBR2_1 target site
  source 1..20: mol_type = other RNA
gggaagctctttcacttcgg

SEQ ID NO: 301 - RNA, 23 nt, synthetic construct
  misc_feature 1..23: note = TGFBR2_2 target site
  source 1..23: mol_type = other RNA
actgtaagtcgctgcctgagtgg

SEQ ID NO: 302 - RNA, 20 nt, synthetic construct
  misc_feature 1..20: note = TGFBR2_3 target site
  source 1..20: mol_type = other RNA
gagcgggtccaccaacccgc

SEQ ID NO: 303 - RNA, 20 nt, synthetic construct
  modified_base 3..4: mod_base = OTHER; note = thymine
  modified_base 9: mod_base = OTHER; note = thymine
  modified_base 16: mod_base = OTHER; note = thymine
  misc_feature 1..20: note = TGFBR2_1 gRNA spacer
  source 1..20: mol_type = other RNA
acttcaactcagcgctgcgg

SEQ ID NO: 304 - RNA, 20 nt, synthetic construct
  modified_base 3: mod_base = OTHER; note = thymine
  modified_base 9: mod_base = OTHER; note = thymine
  modified_base 12: mod_base = OTHER; note = thymine
  modified_base 14: mod_base = OTHER; note = thymine
  misc_feature 1..20: note = TGFBR2_2 gRNA spacer
  source 1..20: mol_type = other RNA
agtccggctcctgtcccgag

SEQ ID NO: 305 - RNA, 20 nt, synthetic construct
  modified_base 6: mod_base = OTHER; note = thymine
  modified_base 9: mod_base = OTHER; note = thymine
  modified_base 20: mod_base = OTHER; note = thymine
  misc_feature 1..20: note = TGFBR2_3 gRNA spacer
  source 1..20: mol_type = other RNA
gaaactcctcgccaacagct

SEQ ID NO: 306 - RNA, 20 nt, synthetic construct
  misc_feature 1..20: note = TGFBR2_4 target site
  source 1..20: mol_type = other RNA
acttcaactcagcgctgcgg

SEQ ID NO: 307 - RNA, 20 nt, synthetic construct
  misc_feature 1..20: note = TGFBR2_5 target site
  source 1..20: mol_type = other RNA
agtccggctcctgtcccgag

SEQ ID NO: 308 - RNA, 20 nt, synthetic construct
  misc_feature 1..20: note = TGFBR2_6 target site
  source 1..20: mol_type = other RNA
gaaactcctcgccaacagct

SEQ ID NO: 309 - RNA, 20 nt, synthetic construct
  modified_base 2: mod_base = OTHER; note = thymine
  modified_base 13: mod_base = OTHER; note = thymine
  misc_feature 1..20: note = TGFBR2_4 gRNA spacer
  source 1..20: mol_type = other RNA
gtcccgagcgggtgcacgcg

SEQ ID NO: 310 - RNA, 20 nt, synthetic construct
  modified_base 2: mod_base = OTHER; note = thymine
  modified_base 8: mod_base = OTHER; note = thymine
  modified_base 11: mod_base = OTHER; note = thymine
  modified_base 13: mod_base = OTHER; note = thymine
  misc_feature 1..20: note = TGFBR2_5 gRNA spacer
  source 1..20: mol_type = other RNA
gtccggctcctgtcccgagc

SEQ ID NO: 311 - RNA, 20 nt, synthetic construct
  modified_base 11: mod_base = OTHER; note = thymine
  misc_feature 1..20: note = TGFBR2_6 gRNA spacer
  source 1..20: mol_type = other RNA
cccgagcgggtgcacgcgcg

SEQ ID NO: 312 - RNA, 20 nt, synthetic construct
  misc_feature 1..20: note = dSpCas9-KOX1(2-99)
  source 1..20: mol_type = other RNA
gtcccgagcgggtgcacgcg

SEQ ID NO: 313 - RNA, 20 nt, synthetic construct
  misc_feature 1..20: note = dSpCas9-KOX1(1-72)
  source 1..20: mol_type = other RNA
gtccggctcctgtcccgagc

SEQ ID NO: 314 - RNA, 20 nt, synthetic construct
  misc_feature 1..20: note = dSpCas9-ZIM3
  source 1..20: mol_type = other RNA
cccgagcgggtgcacgcgcg

SEQ ID NO: 315 - protein, 1789 aa, synthetic construct
  REGION 1..1789: note = dSpCas9-ZNF324
MDYKDHDGDYKDHDIDYKDDDDKHVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 316 - protein, 1765 aa, synthetic construct
  REGION 1..1765: note = dSpCas9-EZH2
MDYKDHDGDYKDHDIDYKDDDDKHVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEPPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 317 - protein, 1792 aa, synthetic construct
  REGION 1..1792: note = D3AL-XTEN80-dSpCas9-KOX1(2-99)
MDYKDHDGDYKDHDIDYKDDDDKHVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 318 - protein, 1784 aa, synthetic construct
  REGION 1..1784: note = D3AL-XTEN80-dSpCas9-KOX1(1-72)
MDYKDHDGDYKDHDIDYKDDDDKHVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSGPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 319 - protein, 2458 aa, synthetic construct
  REGION 1..2458: note = D3AL-XTEN80-dSpCas9-ZIM3
MDYKDHDGDYKDHDIDYKDDDDKHVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 320 - protein, 2412 aa, synthetic construct
  REGION 1..2412: note = D3AL-XTEN80-dSpCas9-ZNF324
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 321 - protein, 2388 aa, synthetic construct
  REGION 1..2388: note = D3AL-XTEN80-dSpCas9-EZH2
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEPPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 322 - protein, 2415 aa, synthetic construct
  REGION 1..2415: note = D3AL-XTEN80-KOX1(2-99)-dSpCas9
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 323 - protein, 2407 aa, synthetic construct
  REGION 1..2407: note = D3AL-XTEN80-KOX1(1-72)-dSpCas9
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSGPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 324 - protein, 3081 aa, synthetic construct
  REGION 1..3081: note = D3AL-XTEN80-ZIM3-dSpCas9
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 325 - protein, 2403 aa, synthetic construct
  REGION 1..2403: note = D3AL-XTEN80-ZNF324-dSpCas9
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEDAKSLTAWSRTLVTFKDVFVDFTREEWKLLDTAQQILYRNVMLENYKNLVSLGYQLTKPDVILRLEKGEEPWLVEREIHQETHPDSETAFEIKSSVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 326 - protein, 2379 aa, synthetic construct
  REGION 1..2379: note = D3AL-XTEN80-EZH2-dSpCas9
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDAKSLTAWSRTLVTFKDVFVDFTREEWKLLDTAQQIVYRNVMLENYKNLVSLGYQLTKPDVILRLEKGEEPNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 327 - protein, 2406 aa, synthetic construct
  REGION 1..2406: note = D3BL-XTEN80-dSpCas9-KOX1(2-99)
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 328 - protein, 2398 aa, synthetic construct
  REGION 1..2398: note = D3BL-XTEN80-dSpCas9-KOX1(1-72)
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEAFEDVAVYFSQEEWGLLDTAQRALYRRVMLDNFALVASLGLSTSRPRVVIQLERGEEPWVPSGTDTTLSRTTYRRRNPGSWSLTEDRDVSGNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 329 - protein, 3072 aa, synthetic construct
  REGION 1..3072: note = D3BL-XTEN80-dSpCas9-ZIM3
MDYKDHDGDYKDHDIDYKDDDDKHVNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 330 - protein, 2432 aa, synthetic construct
  REGION 1..2432: note = D3BL-XTEN80-dSpCas9-ZNF324
MDYKDHDGDYKDHDIDYKDDDDKHVVLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 331 - protein, 2408 aa, synthetic construct
  REGION 1..2408: note = D3BL-XTEN80-dSpCas9-EZH2
MDYKDHDGDYKDHDIDYKDDDDKHVVLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEPPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 332 - protein, 2435 aa, synthetic construct
  REGION 1..2435: note = dSpCas9-KOX1(2-99) - No FLAG, P2A-mCherry
MDYKDHDGDYKDHDIDYKDDDDKHVVLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 333 - protein, 2427 aa, synthetic construct
  REGION 1..2427: note = dSpCas9-KOX1(1-72) - No FLAG, P2A-mCherry
MDYKDHDGDYKDHDIDYKDDDDKHVVLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSGPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 334 - protein, 3101 aa, synthetic construct
  REGION 1..3101: note = dSpCas9-ZIM3 - No FLAG, P2A-mCherry
MDYKDHDGDYKDHDIDYKDDDDKHVVLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPPKKKRKVASATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 335 - protein, 1506 aa, synthetic construct
  REGION 1..1506: note = dSpCas9-ZNF324 - No FLAG, P2A-mCherry
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 336 - protein, 1482 aa, synthetic construct
  REGION 1..1482: note = dSpCas9-EZH2 - No FLAG, P2A-mCherry
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEPPKKKRKV

SEQ ID NO: 337 - protein, 1509 aa, synthetic construct
  REGION 1..1509: note = D3AL-XTEN80-dSpCas9-KOX1(2-99) - No FLAG, P2A-mCherry
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLPKKKRKV

SEQ ID NO: 338 - protein, 1501 aa, synthetic construct
  REGION 1..1501: note = D3AL-XTEN80-dSpCas9-KOX1(1-72) - No FLAG, P2A-mCherry
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSGPKKKRKV

SEQ ID NO: 339 - protein, 2175 aa, synthetic construct
  REGION 1..2175: note = D3AL-XTEN80-dSpCas9-ZIM3 - No FLAG, P2A-mCherry
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPPKKKRKV

SEQ ID NO: 340 - protein, 2130 aa, synthetic construct
  REGION 1..2130: note = D3AL-XTEN80-dSpCas9-ZNF324 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 341 - protein, 2106 aa, synthetic construct
  REGION 1..2106: note = D3AL-XTEN80-dSpCas9-EZH2 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEPPKKKRKV

SEQ ID NO: 342 - protein, 2133 aa, synthetic construct
  REGION 1..2133: note = D3AL-XTEN80-KOX1(2-99)-dSpCas9 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLPKKKRKV

SEQ ID NO: 343 - protein, 2125 aa, synthetic construct
  REGION 1..2125: note = D3AL-XTEN80-KOX1(1-72)-dSpCas9 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSGPKKKRKV

SEQ ID NO: 344 - protein, 2799 aa, synthetic construct
  REGION 1..2799: note = D3AL-XTEN80-ZIM3-dSpCas9 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPPKKKRKV

SEQ ID NO: 345 - protein, 2121 aa, synthetic construct
  REGION 1..2121: note = D3AL-XTEN80-ZNF324-dSpCas9 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEDAKSLTAWSRTLVTFKDVFVDFTREEWKLLDTAQQILYRNVMLENYKNLVSLGYQLTKPDVILRLEKGEEPWLVEREIHQETHPDSETAFEIKSSVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKK

SEQ ID NO: 346 - protein, 2097 aa, synthetic construct
  REGION 1..2097: note = D3AL-XTEN80-EZH2-dSpCas9 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDAKSLTAWSRTLVTFKDVFVDFTREEWKLLDTAQQIVYRNVMLENYKNLVSLGYQLTKPDVILRLEKGEEPNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKK

SEQ ID NO: 347 - protein, 2124 aa, synthetic construct
  REGION 1..2124: note = D3BL-XTEN80-dSpCas9-KOX1(2-99) - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKK

SEQ ID NO: 348 - protein, 2116 aa, synthetic construct
  REGION 1..2116: note = D3BL-XTEN80-dSpCas9-KOX1(1-72) - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEAFEDVAVYFSQEEWGLLDTAQRALYRRVMLDNFALVASLGLSTSRPRVVIQLERGEEPWVPSGTDTTLSRTTYRRRNPGSWSLTEDRDVSGNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKK

SEQ ID NO: 349 - protein, 2790 aa, synthetic construct
  REGION 1..2790: note = D3BL-XTEN80-dSpCas9-ZIM3 - No FLAG, P2A-mCherry
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKK

SEQ ID NO: 350 - protein, 2150 aa, synthetic construct
  REGION 1..2150: note = D3BL-XTEN80-dSpCas9-ZNF324 - No FLAG, P2A-mCherry
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 351 - protein, 2126 aa, synthetic construct
  REGION 1..2126: note = D3BL-XTEN80-dSpCas9-EZH2 - No FLAG, P2A-mCherry
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEPPKKKRKV

SEQ ID NO: 352 - protein, 2153 aa, synthetic construct
  REGION 1..2153: note = P2A
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLPKKKRKV

SEQ ID NO: 353 - protein, 2145 aa, synthetic construct
  REGION 1..2145: note = mCherry
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSGPKKKRKV

SEQ ID NO: 354 - protein, 2819 aa, synthetic construct
  REGION 1..2819: note = P2A-mCherry
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPPKKKRKV

SEQ ID NO: 355 - protein, 19 aa, synthetic construct
  REGION 1..19: note = KRAB from KOX1(2-99)
ATNFSLLKQAGDVEENPGP

SEQ ID NO: 356 - protein, 236 aa, synthetic construct
  REGION 1..236: note = KRAB from KOX1(1-72)
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 357 - protein, 255 aa, synthetic construct
  REGION 1..255: note = KRAB from ZIM3
ATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 358 - protein, 96 aa, synthetic construct
  REGION 1..96: note = KRAB from ZNF324
DAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 359 - protein, 72 aa, synthetic construct
  REGION 1..72: note = EZH2 domain
MDAKSLTAWSRTLVTFKDVFVDFTREEWKLLDTAQQIVYRNVMLENYKNLVSLGYQLTKPDVILRLEKGEEP

SEQ ID NO: 360 - protein, 99 aa, synthetic construct
  REGION 1..99: note = DNMT3B domain
NNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 361 - protein, 91 aa, synthetic construct
  REGION 1..91: note = Linker - XTEN80
AFEDVAVYFSQEEWGLLDTAQRALYRRVMLDNFALVASLGLSTSRPRVVIQLERGEEPWVPSGTDTTLSRTTYRRRNPGSWSLTEDRDVSG

SEQ ID NO: 362 - protein, 745 aa, synthetic construct
  REGION 1..745: note = Linker - 20AA
GQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 363 - protein, 321 aa, synthetic construct
  REGION 1..321: note = DNMT3B-DNMT3L fusion domain
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACE

SEQ ID NO: 364 - protein, 80 aa, synthetic construct; identical to SEQ ID NO: 270
  REGION 1..80: note = FLAG tag (AA)

SEQ ID NO: 365 - protein, 20 aa, synthetic construct
  REGION 1..20: note = dSpCas9-KOX1(2-99) - minimal
STGGSGGSGGSGGSGGSGRP

SEQ ID NO: 366 - protein, 563 aa, synthetic construct
  REGION 1..563: note = dSpCas9-KOX1(1-72) - minimal
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 367 - protein, 22 aa, synthetic construct
  REGION 1..22: note = dSpCas9-ZIM3 - minimal
DYKDHDGDYKDHDIDYKDDDDK

SEQ ID NO: 368 - protein, 1484 aa, synthetic construct
  REGION 1..1484: note = dSpCas9-ZNF324 - minimal
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 369 - protein, 1460 aa, synthetic construct
  REGION 1..1460: note = dSpCas9-EZH2 - minimal
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEP

SEQ ID NO: 370 - protein, 1487 aa, synthetic construct
  REGION 1..1487: note = D3AL-XTEN80-dSpCas9-KOX1(2-99) - minimal
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 371 - protein, 1479 aa, synthetic construct
  REGION 1..1479: note = D3AL-XTEN80-dSpCas9-KOX1(1-72) - minimal
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSG

SEQ ID NO: 372 - protein, 2153 aa, synthetic construct
  REGION 1..2153: note = D3AL-XTEN80-dSpCas9-ZIM3 - minimal
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 373 - protein, 2123 aa, synthetic construct
  REGION 1..2123: note = D3AL-XTEN80-dSpCas9-ZNF324 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 374 - protein, 2099 aa, synthetic construct
  REGION 1..2099: note = D3AL-XTEN80-dSpCas9-EZH2 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEP

SEQ ID NO: 375 - protein, 2126 aa, synthetic construct
  REGION 1..2126: note = D3AL-XTEN80-KOX1(2-99)-dSpCas9 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 376 - protein, 2118 aa, synthetic construct
  REGION 1..2118: note = D3AL-XTEN80-KOX1(1-72)-dSpCas9 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASAFEDVAVYFSQEEWGLLDTAQRALYRRVMLDNFALVASLGLSTSRPRVVIQLERGEEPWVPSGTDTTLSRTTYRRRNPGSWSLTEDRDVSG

SEQ ID NO: 377 - protein, 2792 aa, synthetic construct
  REGION 1..2792: note = D3AL-XTEN80-ZIM3-dSpCas9 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASSTGGSGGSGGSGGSGGSGRPGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 378 - protein, 2102 aa, synthetic construct
  REGION 1..2102: note = D3AL-XTEN80-ZNF324-dSpCas9 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEDAKSLTAWSRTLVTFKDVFVDFTREEWKLLDTAQQILYRNVMLENYKNLVSLGYQLTKPDVILRLEKGEEPWLVEREIHQETHPDSETAFEIKSSVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 379 - protein, 2078 aa, synthetic construct
  REGION 1..2078: note = D3AL-XTEN80-EZH2-dSpCas9 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDAKSLTAWSRTLVTFKDVFVDFTREEWKLLDTAQQIVYRNVMLENYKNLVSLGYQLTKPDVILRLEKGEEPNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 380 - protein, 2105 aa, synthetic construct
  REGION 1..2105: note = D3BL-XTEN80-dSpCas9-KOX1(2-99) - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 381 - protein, 2097 aa, synthetic construct
  REGION 1..2097: note = D3BL-XTEN80-dSpCas9-KOX1(1-72) - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEAFEDVAVYFSQEEWGLLDTAQRALYRRVMLDNFALVASLGLSTSRPRVVIQLERGEEPWVPSGTDTTLSRTTYRRRNPGSWSLTEDRDVSGNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 382 - protein, 2771 aa, synthetic construct
  REGION 1..2771: note = D3BL-XTEN80-dSpCas9-ZIM3 - minimal
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIPSTGGSGGSGGSGGSGGSGRPNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 383 - protein, 2143 aa, synthetic construct
  REGION 1..2143: note = D3BL-XTEN80-dSpCas9-ZNF324 - minimal
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 384 - protein, 2119 aa, synthetic construct
  REGION 1..2119: note = D3BL-XTEN80-dSpCas9-EZH2 - minimal
VLRRRKDWNMRLQDFFTTDPDLEEFQEPPKLYPAIPAAKRRPIRVLSLFDGIATGYLVLKELGIKVEKYIASEVCAESIAVGTVKHEGQIKYVNDVRKITKKNIEEWGPFDLVIGGSPCNDLSNVNPARKGLYEGTGRLFFEFYHLLNYTRPKEGDNRPFFWMFENVVAMKVNDKKDISRFLACNPVMIDAIKVSAAHRARYFWGNLPGMNRPVMASKNDKLELQDCLEFSRTAKLKKVQTITTKSNSIRQGKNQLFPVVMNGKDDVLWCTELERIFGFPAHYTDVSNMGRGARQKLLGRSWSVPVIRHLFAPLKDYFACESSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSENPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASMDAKSLTAWSRTLVTFKDVFVDFTREEWKLLDTAQQIVYRNVMLENYKNLVSLGYQLTKPDVILRLEKGEEP

SEQ ID NO: 385 - protein, 112 aa, synthetic construct
  REGION 1..112: note = Bipartite activator - PYGO1 NCOA3 (aa)
PCGICTNEVNDDQDAILCEASCQKWFHRICTGMTETAYGLLTAEASAVWGCDTCMADNGGGGSEGQSDERALLDQLHTLLSNTDATGLEEIDRALGIPELVNQGQALEPKQD

SEQ ID NO: 386 - protein, 92 aa, synthetic construct
  REGION 1..92: note = Bipartite activator - NOTCH2 NCOA3 (aa)
DIDECASNPCRKGATCINGVNGFRCICPEGPHHPSCYNGGGGSEGQSDERALLDQLHTLLSNTDATGLEEIDRALGIPELVNQGQALEPKQD

SEQ ID NO: 387 - protein, 103 aa, synthetic construct
  REGION 1..103: note = Bipartite activator - NCOA3 NCOA3 (aa)
EGQSDERALLDQLHTLLSNTDATGLEEIDRALGIPELVNQGQALEPKQDGGGGSEGQSDERALLDQLHTLLSNTDATGLEEIDRALGIPELVNQGQALEPKQD

SEQ ID NO: 388 - protein, 131 aa, synthetic construct
  REGION 1..131: note = Bipartite activator - HSH2D NCOA3 (aa)
WFHGAISREDAENLLESQPLGSFLIRVSHSHVGYTLSYKAQSSCCHFMVKLLDDGTFMIPGEKVAHTSLDALVTFHNGGGGSEGQSDERALLDQLHTLLSNTDATGLEEIDRALGIPELVNQGQALEPKQD

SEQ ID NO: 389 - protein, 96 aa, synthetic construct
  REGION 1..96: note = Bipartite activator - FOXO3 NCOA3 (aa)
HEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 390 - protein, 102 aa, synthetic construct
  REGION 1..102: note = Bipartite activator - NCOA2 NCOA3 (aa)
ESPSDEGALLDQLYLALRNFDGLEEIDRALGIPELVSQSQAVDPEQFNGGGGSEGQSDERALLDQLHTLLSNTDATGLEEIDRALGIPELVNQGQALEPKQD

SEQ ID NO: 391 - protein, 117 aa, synthetic construct
  REGION 1..117: note = Bipartite activator - ENL NCOA3 (aa)
AYTDELVELHRRLMALRERNVLQQIVNLIEETGHFNVTNTTFDFDLFSLDETTVRKLQSCLENGGGGSEGQSDERALLDQLHTLLSNTDATGLEEIDRALGIPELVNQGQALEPKQD

SEQ ID NO: 392 - protein, 105 aa, synthetic construct
  REGION 1..105: note = Bipartite activator - PYGO1 FOXO3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSL

SEQ ID NO: 393 - protein, 85 aa, synthetic construct
  REGION 1..85: note = Bipartite activator - NOTCH2 FOXO3 (aa)
DIDECASNPCRKGATCINGVNGFRCICPEGPHHPSCYNGGGGSHEKFPSDLDLDMFNGSLECDMESIIRSELMDADGLDFNFDSL

SEQ ID NO: 394 - protein, 97 aa, synthetic construct
  REGION 1..97: note = Bipartite activator - NCOA3 FOXO3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSL

SEQ ID NO: 395 - protein, 124 aa, synthetic construct
  REGION 1..124: note = Bipartite activator - HSH2D FOXO3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSL

SEQ ID NO: 396 - protein, 90 aa, synthetic construct
  REGION 1..90: note = Bipartite activator - FOXO3 FOXO3 (aa)
HEKFPSDLDLDMFNGSLECDMESIIRSELMDADGLDFNFDSLNGGGGSHEKFPSDLDLDMFNGSLECDMESIIRSELMDADGLDFNFDSL

SEQ ID NO: 397 - protein, 95 aa, synthetic construct
  REGION 1..95: note = Bipartite activator - NCOA2 FOXO3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSL

SEQ ID NO: 398 - protein, 110 aa, synthetic construct
  REGION 1..110: note = Bipartite activator - ENL FOXO3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSL

SEQ ID NO: 399 - protein, 159 aa, synthetic construct
  REGION 1..159: note = Tripartite activator - PYGO1 FOXO3 NCOA3 (aa)
PCGICTNEVNDDQDAILCEASCQKWFHRICTGMTETAYGLLTAEASAVWGCDTCMADNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 400 - protein, 139 aa, synthetic construct
  REGION 1..139: note = Tripartite activator - NOTCH2 FOXO3 NCOA3 (aa)
DIDECASNPCRKGATCINGVNGFRCICPEGPHHPSCYNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 401 - protein, 151 aa, synthetic construct
  REGION 1..151: note = Tripartite activator - NCOA3 FOXO3 NCOA3 (aa)
EGQSDERALLDQLHTLLSNTDATGLEEIDRALGIPELVNQGQALEPKQDNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 402 - protein, 178 aa, synthetic construct
  REGION 1..178: note = Tripartite activator - HSH2D FOXO3 NCOA3 (aa)
WFHGAISREDAENLLESQPLGSFLIRVSHSHVGYTLSYKAQSSCCHFMVKLLDDGTFMIPGEKVAHTSLDALVTFHNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 403 - protein, 144 aa, synthetic construct
  REGION 1..144: note = Tripartite activator - FOXO3 FOXO3 NCOA3 (aa)
HEKFPSDLDLDMFNGSLECDMESIIRSELMDADGLDFNFDSLNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 404 - protein, 149 aa, synthetic construct
  REGION 1..149: note = Tripartite activator - NCOA2 FOXO3 NCOA3 (aa)
ESPSDEGALLDQLYLALRNFDGLEEIDRALGIPELVSQSQAVDPEQFNGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 405 - protein, 164 aa, synthetic construct
  REGION 1..164: note = Tripartite activator - ENL FOXO3 NCOA3 (aa)
AYTDELVELHRRLMALRERNVLQQIVNLIEETGHFNVTNTTFDFDLFSLDETTVRKLQSCLENGGGGSHEKFPSDLDLDMFNGSLECDMESIIRSELMDADGLDFNFDSLGGGGSEGQSDERALLDQLHTLLSNTDATGLEEIDRALGIPELVNQGQALEPKQD

SEQ ID NO: 406 - protein, 144 aa, synthetic construct
  REGION 1..144: note = Tripartite activator - NC OA3, FOXO3, FOXO3 (aa)
EGQSDERALLDQLHTLLSNTDATGLEEIDRALGIPELVNQGQALEPKQDGGGGSHEKFPSDLDLDMFNGSLECDMESIIRSELMDADGLDFNFDSLNGGGGSHEKFPSDLDLDMFNGSLECDMESIIRSELMDADGLDFNFDSL

SEQ ID NO: 407 - protein, 80 aa, synthetic construct
  REGION 1..80: note = DPOLA (aa)
KSKKGRQEALERLKKAKAGEKYKYEVEDFTGVYEEVDEEQYSKLVQARQDDDWIVDDDGIGYVEDGREIFDDDLEDDALD

SEQ ID NO: 408 - protein, 80 aa, synthetic construct
  REGION 1..80: note = ENL (aa)
SKPEKILKKGTYDKAYTDELVELHRRLMALRERNVLQQIVNLIEETGHFNVTNTTFDFDLFSLDETTVRKLQSCLEAVAT

SEQ ID NO: 409 - protein, 80 aa, synthetic construct
  REGION 1..80: note = FOXO3 (aa)
DSLSGSSLYSTSANLPVMGHEKFPSDLDLDMFNGSLECDMESIIRSELMDADGLDFNFDSLISTQNVVGLNVGNFTGAKQ

SEQ ID NO: 410 - protein, 80 aa, synthetic construct
  REGION 1..80: note = HSH2D (aa)
PEWFHGAISREDAENLLESQPLGSFLIRVSHSHVGYTLSYKAQSSCCHFMVKLLDDGTFMIPGEKVAHTSLDALVTFHQQ

SEQ ID NO: 411 - protein, 80 aa, synthetic construct
  REGION 1..80: note = NCOA2 (aa)
PFGSSPDDLLCPHPAAESPSDEGALLDQLYLALRNFDGLEEIDRALGIPELVSQSQAVDPEQFSSQDSNIMLEQKAPVFP

SEQ ID NO: 412 - protein, 80 aa, synthetic construct
  REGION 1..80: note = NCOA3 (aa)
LRNSLDDLVGPPSNLEGQSDERALLDQLHTLLSNTDATGLEEIDRALGIPELVNQGQALEPKQDAFQGQEAAVMMDQKAG

SEQ ID NO: 413 - protein, 80 aa, synthetic construct
  REGION 1..80: note = PSA1 (aa)
FRNQYDNDVTVWSPQGRIHQIEYAMEAVKQGSATVGLKSKTHAVLVALKRAQSELAAHQKKILHVDNHIGISIAGLTADA

SEQ ID NO: 414 - protein, 80 aa, synthetic construct
  REGION 1..80: note = PYGO1 (aa)
RHGHSSSDPVYPCGICTNEVNDDQDAILCEASCQKWFHRICTGMTETAYGLLTAEASAVWGCDTCMADKDVQLMRTRETF

SEQ ID NO: 415 - protein, 80 aa, synthetic construct
  REGION 1..80: note = RBM39 (aa)
GPMRLYVGSLHFNITEDMLRGIFEPFGRIESIQLMMDSETGRSKGYGFITFSDSECAKKALEQLNGFELAGRPMKVGHVT

SEQ ID NO: 416 - protein, 80 aa, synthetic construct
  REGION 1..80: note = HERC2 (aa)
TLIRKADLENHNKDGGFWTVIDGKVYDIKDFQTQSLTGNSILAQFAGEDPVVALEAALQFEDTRESMHAFCVGQYLEPDQ

SEQ ID NO: 417 - protein, 80 aa, synthetic construct
  REGION 1..80: note = ZNF473 (AA)
AEEFVTLKDVGMDFTLGDWEQLGLEQGDTFWDTALDNCQDLFLLDPPRPNLTSHPDGSEDLEPLAGGSPEATSPDVTETK

SEQ ID NO: 418 - protein, 80 aa, synthetic construct
  REGION 1..80: note = ANM2 (AA)
ECSEAGLLQEGVQPEEFVAIADYAATDETQLSFLRGEKILILRQTTADWWWGERAGCCGYIPANHVGKHVDEYDPEDTWQ

SEQ ID NO: 419 - protein, 80 aa, synthetic construct
  REGION 1..80: note = KIBRA (AA)
PRPELPLPEGWEEARDFDGKVYYIDHTNRTTSWIDPRDRYTKPLTFADCISDELPLGWEEAYDPQVGDYFIDHNTKTTQI

SEQ ID NO: 420 - protein, 80 aa, synthetic construct
  REGION 1..80: note = IKKA (AA)
LVGSSLEGAVTPQTSAWLPPTSAEHDHSLSCVVTPQDGETSAQMIEENLNCLGHLSTIIHEANEEQGNSMMNLDWSWLTE

SEQ ID NO: 421 - protein, 80 aa, synthetic construct
  REGION 1..80: note = APBB1 (AA)
GSPSYGSPEDTDSFWNPNAFETDSDLPAGWMRVQDTSGTYYWHIPTGTTQWEPPGRASPSQGSSPQEESQLTWTGFAHGE

SEQ ID NO: 422 - protein, 80 aa, synthetic construct
  REGION 1..80: note = SMN2 (AA)
GNREEQNLSDLLSPICEVANNIEQNAQENENESQVSTDESENSRSPGNKSDNIKPKSAPWNSFLPPPPPMPGPRLGPGKP

SEQ ID NO: 423 - protein, 80 aa, synthetic construct
  REGION 1..80: note = SERTAD2 (AA)
LMDSLPGNFEITTSTGFLTDLTLDDILFADIDTSMYDFDPCTSSSGTASKMAPVSADDLLKTLAPYSSQPVTPSQPFKMD

SEQ ID NO: 424 - protein, 80 aa, synthetic construct
  REGION 1..80: note = MYBA (AA)
FYIPVQIPGYQYVSPEGNCIEHVQPTSAFIQQPFIDEDPDKEKKIKELEMLLMSAENEVRRKRIPSQPGSFSSWSGSFLM